(19) United States
(12) Patent Application Publication
(10) Pub. No.: US 2024/0272170 A1
(43) Pub. Date: Aug. 15, 2024

(54) COMPOSITIONS AND METHODS FOR POLYPEPTIDE ANALYSIS

(71) Applicant: Quantum-Si Incorporated, Branford, CT (US)

(72) Inventors: Brian Reed, Madison, CT (US); Manjula Pandey, Guilford, CT (US); Marco Ribezzi-Crivellari, Paris (FR); Douglas Pike, Guilford, CT (US); Jennifer Tullman, Carmel, IN (US); Kristin Blacklock, Holmdel, NJ (US); Adeline Pichard-Kostuch, Paris (FR); Ahmed Rehan, Paris (FR); Mohammad Wadud Bhuiya, Olivette, MO (US)

(21) Appl. No.: 18/365,185

(22) Filed: Aug. 3, 2023

Related U.S. Application Data
(60) Provisional application No. 63/395,328, filed on Aug. 4, 2022.

(51) Int. Cl. G01N 33/68; C07K 14/37

(52) U.S. Cl. CPC G01N 33/6824; C07K 14/37

(57) ABSTRACT

There is provided amino acid recognizers with improved binding properties, allowing for more structural information to be obtained from polypeptides based on the kinetics of on-off binding between recognizer and polypeptide. An amino acid recognizer may comprise an amino acid binding protein with an engineered binding pocket having one or more modifications relative to a homologous protein. The modified binding pocket may increase the number of interactions formed between the binding pocket and an amino acid ligand as compared to an unmodified binding pocket of the homologous protein, increase the number of types of amino acid ligands capable of being detectably bound as compared to an unmodified binding pocket of the homologous protein, and improve the kinetics of binding (e.g., K_{D}, k_{off}, kₒₙ) toward one or more types of amino acid ligands, which advantageously increases the amount of, or confidence in, structural information that may be derived from polypeptide analysis.

### CROSS-REFERENCE TO RELATED APPLICATIONS

This application claims priority under 35 U.S.C. § 119(e) to U.S. Provisional Patent Application No. 63/395,328, filed Aug. 4, 2022, which is hereby incorporated by reference in its entirety.

### REFERENCE TO AN ELECTRONIC SEQUENCE LISTING

The contents of the electronic sequence listing (R070870157US01-SEQ-JIB.xml; Size: 1,140,924 bytes; and Date of Creation: Aug. 3, 2023) is herein incorporated by reference in its entirety.

### BACKGROUND

Proteins represent the fundamental building blocks of life, driving key biological and cellular processes. Protein function is driven by its structure, including its sequence. In adjacent fields, like genomics, advances in sequencing technology have proven extremely valuable in improving our understanding of the progression of complex human disease. Applying similar approaches to proteomics has been difficult because of the scale, dynamic range, and inability to amplify the source.

### SUMMARY

In some embodiments, there is provided a recombinant or synthetic amino acid binding protein having an amino acid sequence that is at least 80% identical to SEQ ID NO: 1, wherein the amino acid sequence comprises an amino acid substitution at one or more positions corresponding to E22, R31, L39, N41, D42, D43, D44, H45, T46, Y47, V50, Q55, P62, E63, L68, A69, V72, D73, Q75, Y100, and M111 of SEQ ID NO: 1.

In some embodiments, there is provided a recombinant or synthetic amino acid binding protein comprising a structure of Formula (I) or a structural equivalent thereof: β1-α1-α2-β2-α3-β3 (I), wherein: each of β1, β2, and β3 is a beta-strand; each of α1, α2, and α3 is an alpha-helix; each instance of “-” is a loop; and at least a portion of each of α1, α2, the loop between β1 and α1, and the loop between α3 and β3 form a binding pocket for an amino acid ligand, wherein the binding pocket comprises one or more of the following: (i) a volume of approximately 170 Å³, (ii) an electrostatic potential of −3.0 RTe_{c}⁻¹ or less, (iii) negatively charged side-chains in at least 35% of amino acids that form the binding pocket, (iv) a plurality of hydrogen bond acceptors configured to form one or more hydrogen bonds in the presence of the amino acid ligand, and (v) a plurality of van der Waals contact positions configured to form van der Waals interactions in the presence of the amino acid ligand.

In some embodiments, there is provided a recombinant or synthetic amino acid binding protein having an amino acid sequence that is at least 80% identical to SEQ ID NO: 2, wherein the amino acid sequence comprises an amino acid substitution at one or more positions corresponding to G19, K26, S29, F30, D31, D32, T33, C34, V35, T47, G48, T53, T54, T57, E58, F59, N61, 163, D65, D68, E70, A71, H74, and T75 of SEQ ID NO: 2.

In some embodiments, there is provided a recombinant or synthetic amino acid binding protein comprising a structure of Formula (II) or a structural equivalent thereof: β1-α1-β2-α2-α3 (II), wherein: each of β1 and β2 is a beta-strand; each of α1, α2, and α3 is an alpha-helix; each instance of “-” is a loop; and at least a portion of each of α2, the loop between β1 and α1, and the loop between β2 and α2 form a binding pocket for an amino acid ligand, wherein the binding pocket comprises one or more of the following: (i) a volume of approximately 200 Å³, (ii) an electrostatic potential of −3.0 RTe_{c}⁻¹ or less, (iii) a plurality of hydrogen bond acceptors configured to form one or more hydrogen bonds in the presence of the amino acid ligand, and (iv) a plurality of van der Waals contact positions configured to form van der Waals interactions in the presence of the amino acid ligand.

In some embodiments, there is provided a recombinant or synthetic amino acid binding protein having an amino acid sequence that is at least 80% identical to SEQ ID NO: 3, wherein the amino acid sequence comprises an amino acid substitution at one or more positions corresponding to S22, C23, Y24, C25, E26, S39, W75, D76, Y77, H78, C85, N120, H145, and M146 of SEQ ID NO: 3.

In some embodiments there is provided a recombinant or synthetic amino acid binding protein comprising a structure of Formula (III) or a structural equivalent thereof: α1-α2-α31-β2-β3-β4-β5-α4-β6α5-α6 (III), wherein: each of α1, α2, 3, α4, α5, and α6 is an alpha-helix; each of β1, β2, β3, β4, β5, and β6 is a beta-strand; each instance of “-” is a loop; and at least a portion of each of α2, β3, β4, α5, the loop between α1 and α2, and the loop between β3 and β34 form a binding pocket for an amino acid ligand, wherein the binding pocket comprises one or more of the following: (i) a volume of approximately 160 Å³, (ii) an electrostatic potential of −2.0 RTe_{c}⁻¹ or less, (iii) a plurality of hydrogen bond acceptors or donors configured to form one or more hydrogen bonds in the presence of the amino acid ligand, (iv) a plurality of van der Waals contact positions configured to form van der Waals interactions in the presence of the amino acid ligand, and (v) at least one negatively charged amino acid and at least one positively charged amino acid.

In some embodiments, there is provided an amino acid recognizer comprising a polypeptide having at least a first amino acid binding protein and a second amino acid binding protein joined end-to-end, wherein the first and second amino acid binding proteins are separated by a linker comprising at least two amino acids, wherein at least one of the first and second amino acid binding proteins is an amino acid binding protein according to any of the aspects of the technology described herein.

In some embodiments, there is provided an amino acid recognizer comprising a polypeptide having an amino acid binding protein and a labeled protein joined end-to-end, wherein the amino acid binding protein and the labeled protein are separated by a linker comprising at least two amino acids, wherein the amino acid binding protein is an amino acid binding protein according to any of the aspects of the technology described herein.

In some embodiments, there is provided composition comprising two or more amino acid recognizers, wherein at least one amino acid recognizer is an amino acid binding protein according to any of the aspects of the technology described herein.

According to some embodiments, there is provided a method of determining at least one chemical characteristic of a polypeptide, the method comprising: contacting a polypeptide with a composition according to any of the aspects of the technology described herein; and monitoring a signal for signal pulses corresponding to interactions between one or more amino acid recognizers and the polypeptide; and determining at least one chemical characteristic of the polypeptide based on a characteristic pattern in the signal.

In some embodiments, there is provided a system comprising: at least one hardware processor; and at least one non-transitory computer-readable storage medium storing processor-executable instructions that, when executed by the at least one hardware processor, cause the at least one hardware processor to perform a method according to any of the aspects of the technology described herein.

In some embodiments, there is provided at least one non-transitory computer-readable storage medium storing processor-executable instructions that, when executed by at least one hardware processor, cause the at least one hardware processor to perform a method according to any of the aspects of the technology described herein.

The details of certain embodiments of the disclosure are set forth in the Detailed Description. Other features, objects, and advantages of the disclosure will be apparent from the Examples, Drawings, and Claims.

### BRIEF DESCRIPTION OF THE DRAWINGS

The accompanying Drawings, which constitute a part of this specification, illustrate several embodiments of the disclosure and together with the accompanying description, serve to explain the principles of the disclosure.

FIG. 1 shows an example overview of real-time dynamic protein sequencing. Protein samples are digested into peptide fragments, immobilized in nanoscale reaction chambers, and incubated with a mixture of freely-diffusing N-terminal amino acid (NAA) recognizers and aminopeptidases that carry out the sequencing process. The labeled recognizers bind on and off to the peptide when one of their cognate NAAs is exposed at the N-terminus, thereby producing characteristic pulsing patterns. The NAA is cleaved by an aminopeptidase, exposing the next amino acid for recognition. The temporal order of NAA recognition and the kinetics of binding enable peptide identification and are sensitive to features that modulate binding kinetics, such as post-translational modifications (PTMs). SEQ ID NO: 1090 (RLIFA) is shown.

FIGS. 2A-2G show an example of NAA recognition and dynamic sequencing. FIGS. 2A-2C show example traces demonstrating single-molecule N-terminal recognition by PS610 (FIG. 2A), PS961 (FIG. 2B), and PS691 (FIG. 2C); scatter plots of the number of pulses per recognition segment (RS) vs RS mean pulse duration (PD) are displayed for each peptide in FIGS. 2A-2C, with median PD indicated. FIG. 2D shows example traces from dynamic sequencing of the synthetic peptide FAAWAAYAAAADDD (SEQ ID NO: 1034); median PD is indicated above each RS. FIGS. 2E-2G show dynamic sequencing of the synthetic peptide LAQFASIAAYASDDD (SEQ ID NO: 1035) using PS610 and PS961. FIG. 2E shows example traces. FIG. 2F shows a scatter plot of RS mean PD vs bin ratio illustrating discrimination of recognizers by bin ratio and NAAs by pulse duration. FIG. 2G shows a scatter plot of the number of pulses per RS vs RS mean PD, grouped by the amino acid label assigned to the RS.

FIGS. 3A-3G show an example of dynamic sequencing of diverse peptides with high-precision kinetic outputs. FIGS. 3A-3E show dynamic sequencing of the peptide DQQRLIFAG (SEQ ID NO: 1036). FIG. 3A shows an example trace. FIG. 3B shows a scatter plot of RS mean PD vs bin ratio. FIG. 3C shows additional example traces of dynamic sequencing of DQQRLIFAG (SEQ ID NO: 1036). FIG. 3D shows distributions of the duration of each RS and non-recognition segment (NRS) acquired during sequencing, with mean durations indicated. FIG. 3E shows kinetic signature plots summarizing the characteristic sequencing behavior of DQQRLIFAG (SEQ ID NO: 1036) peptide. FIGS. 3F-3G show dynamic sequencing of the synthetic peptides DQQIASSRLAASFAAQQYPDDD (SEQ ID NO: 1037) (top), RLAFSALGAADDD (SEQ ID NO: 1038) (middle), and EFIAWLV (SEQ ID NO: 1039) (bottom). FIG. 3F shows example traces for each peptide. FIG. 3G shows corresponding kinetic signature plots.

FIGS. 4A-4E show an example of detection of single amino acid changes and PTMs. FIGS. 4A-4B show dynamic sequencing of synthetic peptides that differ by a single amino acid: RLAFAYPDDD (SEQ ID NO: 1040) (top), RLIFAYPDDD (SEQ ID NO: 1041) (middle), RLVFAYPDDD (SEQ ID NO: 1042) (bottom). FIG. 4A shows example traces. FIG. 4B shows scatter plots of RS mean PD vs bin ratio. FIGS. 4C-4D show detection of oxidized methionine using the peptide RLMFAYPDDD (SEQ ID NO: 1043). FIG. 4C shows distributions of mean PD for leucine; labels indicate populations with leucine followed by methionine (LM) or methionine sulfoxide (LMo). FIG. 4D shows example traces in which methionine is recognized by PS961 and leucine exhibits long PD (top), or in which methionine is not recognized due to oxidation (RLMoFAYPDDD (SEQ ID NO: 1044), where “Mo” is methionine sulfoxide) and leucine exhibits short PD (bottom). FIG. 4E shows scatter plots of RS mean PD vs bin ratio for runs in which oxidation was not controlled (top) or in which methionine was fully oxidized (bottom).

FIGS. 5A-5C show an example of discrimination of peptides in mixtures and mapping peptides to the human proteome. FIG. 5A shows example traces from sequencing a mixture of the peptides DQQRLIFAG (SEQ ID NO: 1036) and RLAFSALGAADDD (SEQ ID NO: 1038) on the same chip; the chip window indicates the location of reaction chambers producing a sequencing readout for each peptide. FIG. 5B shows example traces from the dynamic sequencing of two peptides, DQQRLIFAGK (SEQ ID NO: 1045) (top) and EFIAWLVK (SEQ ID NO: 1046) (bottom), isolated from the recombinant human proteins ubiquitin and GLP-1, respectively. FIG. 5C shows a diagram illustrating identification of the protein ubiquitin as a match to the kinetic signature from DQQRLIFAGK (SEQ ID NO: 1045) peptide in an in silico digest of the human proteome based on kinetic information. IVNFSRLIFHHLK (SEQ ID NO: 1095), DIRLIFSNAK (SEQ ID NO: 1096), GQSRLIFTYGLTNSGK (SEQ ID NO: 1097), DQQRLIFAGK (SEQ ID NO: 1045), and DEHCLRLIFLK (SEQ ID NO: 1098) are also shown.

FIGS. 6A-6F show an example of chip operation. FIG. 6A shows an exploded view of the compact benchtop instrument designed to support the custom semiconductor chip and protein sequencing assay. FIG. 6B shows that the chip achieves electronic rejection by discarding photoelectrons from the pulsed laser before shifting to collect fluorescence photoelectrons from bound NAA recognizers; the timing of the rejection and collection windows cycles between two modes (Bin 1 and Bin 0, example waveforms shown) in alternate frames to provide a bin ratio estimate of the fluorescence lifetime of the dye. FIG. 6C shows that the chip achieves >10,000-fold attenuation of incident laser light within 1 ns from initiation of a rejection mode. FIG. 6D shows example pulses for dyes with short and long fluorescence lifetime, illustrating the difference in signal collection in Bin 0 and Bin 1. FIG. 6E shows distributions of mean RS bin ratio collected for three dyes with different fluorescence lifetime. FIG. 6F shows dye channel identification accuracy increases with the number of pulses captured per RS.

FIGS. 7A-7H show an example of recognizer properties. FIGS. 7A-7E show recognizer kinetic characterization using polarization assays (Example 1, Methods). FIGS. 7A-7B show affinity (K_{D}) (FIG. 7A) and off-rate (k_{off}) (FIG. 7B) of PS610 for peptides with N-terminal phenylalanine, tyrosine, and tryptophan. In FIG. 7B, FAKLK(FITC)DEESILKQ (SEQ ID NO: 1099), YAKLK(FITC)DEESILKQ (SEQ ID NO: 1100), and WAKLK(FITC)DEESILKQ (SEQ ID NO: 1101) are shown. FIG. 7C shows affinity of PS961 for peptides with N-terminal leucine, isoleucine, and valine. FIGS. 7D-7E show affinity of PS691 for a peptide with N-terminal arginine (FIG. 7D) and single-point polarization data measured for peptides with N-terminal arginine, lysine, and histidine (FIG. 7E). FIG. 7F shows binding energy was calculated using a computational model (Example 1, Methods) for peptides of initial sequence LAX and LXA, where X=all 20 amino acids; boxplots show the fraction of total binding energy contributed by the amino acid at position 1 (P1), position 2 (P2), and position 3 (P3), with an exponentially decreasing trend from P1 to P3 (R²>0.97). FIG. 7G shows RS mean PD determined in single-molecule assays for LXA and LAX peptides using PS961 and for FXA and FAX peptides using PS610. FIG. 7H shows the non-polar solvation energy term from the computational binding model with PS961 exhibits high correlation with actual RS mean PD values observed in single-molecule assays with peptides containing N-terminal leucine and varying amino acids at the P2 position. LVFA (SEQ ID NO: 1102), LIFA (SEQ ID NO: 1103), LVAR (SEQ ID NO: 1104), LAFA (SEQ ID NO: 1105), LQAR (SEQ ID NO: 1106), LDAA (SEQ ID NO: 1107), LCAR (SEQ ID NO: 1108), LGAA (SEQ ID NO: 1109), LMFA (SEQ ID NO: 1110), LSAR (SEQ ID NO: 1111), and LEFA (SEQ ID NO: 1112) are shown.

FIGS. 8A-8E show an example of binding and cleavage rates. FIGS. 8A-8B show RS mean interpulse duration (IPD) for PS961 binding to LIF (FIG. 8A) and IFA (FIG. 8B) in dynamic sequencing assays at a concentration of 125 nM or 250 nM; median IPD values are indicated. FIG. 8C shows single exponential decay curves fit to the RS duration distributions for arginine, leucine, isoleucine, and phenylalanine acquired from dynamic sequencing of the synthetic peptide DQQRLIFAG (SEQ ID NO: 1036). FIGS. 8D-8E show that increasing the aminopeptidase concentrations in dynamic sequencing runs of the synthetic peptide DQQRLIFAG (SEQ ID NO: 1036) resulted in decreased NRS (FIG. 8D) and RS (FIG. 8E) durations; median RS duration values are indicated.

FIGS. 9A-9G show an example of kinetic signatures from single amino acid changes and PTMs. FIG. 9A shows kinetic signature plots for three peptides: RLAFAYPDDD (SEQ ID NO: 1040) (top), RLIFAYPDDD (SEQ ID NO: 1041) (middle), and RLVFAYPDDD (SEQ ID NO: 1042) (bottom). FIGS. 9B-9C show incomplete RS information observed in dynamic sequencing of RLIFAYPDDD (SEQ ID NO: 1041) peptide. FIG. 9B shows percentage of reads and example traces of each type of observed deletion of one or more RSs in traces beginning with arginine and ending with tyrosine recognition. RLIFY (SEQ ID NO: 1115) is shown. FIG. 9C shows percentage of reads and example traces of each type of observed truncation of one or more RSs in traces beginning with arginine. FIG. 9D shows affinity of PS961 for a peptide with N-terminal methionine measured a polarization assay (Example 1, Methods). FIG. 9E shows binding energy prediction for peptides with N-terminal methionine (MFAY (SEQ ID NO: 1113)) and methionine sulfoxide (Mo) (MoFAY (SEQ ID NO: 1114)) from computational modeling with PS961 (Example 1, Methods). FIG. 9F shows kinetic signature plots for DQQRLIFAG (SEQ ID NO: 1036) and RLAFSALGAADDD (SEQ ID NO: 1038) peptides mixed and run on the same chip. FIG. 9G shows kinetic signature plots for DQQRLIFAGK (SEQ ID NO: 1045) and EFIAWLVK (SEQ ID NO: 1046) peptides obtained from digestion of recombinant human ubiquitin and GLP-1.

FIGS. 10A-10M show an example of peptide identification using modeled proteome-wide kinetic signatures. FIGS. 10A-10C show heatmaps of predicted pulse durations for PS961 binding tripeptide targets having leucine (FIG. 10A), isoleucine (FIG. 10B), or valine (FIG. 10C) at the N-terminal position. FIGS. 10D-10F show heatmaps of predicted pulse durations for PS610 binding tripeptide targets having phenylalanine (FIG. 10D), tyrosine (FIG. 10E), or tryptophan (FIG. 10F) at the N-terminal position. FIG. 10G shows a heatmap of predicted pulse durations for PS1122 binding tripeptide targets having arginine at the N-terminal position. FIG. 10H shows plots demonstrating high correlation of predicted pulse durations with actual pulse durations from on-chip experiments for PS961 (left plot) and PS610 (right plot). FIGS. 10I-10K show the results from an analysis of the human proteome. In FIG. 10J, the sequences of IL6_HUMAN (SEQ ID NO: 1116), DGISALRK (SEQ ID NO: 1117), SNMCESSK (SEQ ID NO: 1118), EALAENNLNLPK (SEQ ID NO: 1119), DGCFQSGFNEETCLVK (SEQ ID NO: 1120), IITGLLEFEVYLEYLQNRFESSEEQARAVQMSTK (SEQ ID NO: 1121), VLIQFLQK (SEQ ID NO: 1122), DPTTNASLLTK (SEQ ID NO: 1123), and DMTTHLILRSFK (SEQ ID NO: 1124) are shown. In FIG. 10K, ACLILRSIEELK (SEQ ID NO: 1125), DMTTHLILRSFK (SEQ ID NO: 1124), SDSRNTLILRCK (SEQ ID NO: 1126), DSSHQISALVLRAQASEILLEELQQGLSQAK (SEQ ID NO: 1127), ARTVGIEELILRIqESK (SEQ ID NO: 1128), STLVLRCHRRRK (SEQ ID NO: 1129), DSPQEPLVLRLK (SEQ ID NO: 1130), and DLVLRATK (SEQ ID NO: 1131) are shown.

FIGS. 10L-10M show the results from an analysis of the E. coli proteome. In FIG. 10M, sequences for SSUA_ECOLI (SEQ ID NO: 1132), LALAGLLSVSTFAVAAESSPEALRIGYQK (SEQ ID NO: 1133), GSSSHNLLLRALRQAGLK (SEQ ID NO: 1134), DPYYSAALLQGGVRVLK (SEQ ID NO: 1135), DLNQTGSFYLAARPYAEK (SEQ ID NO: 1136), DLFYENRLVPK (SEQ ID NO: 1137), and DIRQRIWQPLEGK (SEQ ID NO: 1138) are shown.

FIGS. 11A-11D show example results from a selection and analysis of N-terminal alanine and valine binding variants. FIG. 11A shows results from FACS selections over 3 rounds. FIG. 11B shows results from one round of selection of an error prone PCR library mixture. FIG. 11C shows an example depiction of the presumed binding of an alanine peptide to a variant of PS557. FIG. 11D shows example results from fluorescence polarization studies comparing kinetics of N-terminal alanine peptide binding for selected PS557 variants.

FIGS. 12A-12B show example results from the rational design of recognizer variants of PS557. FIG. 12A shows a heatmap showing enrichment of mutations in the PS557 protein. FIG. 12B shows binding assay traces of selected candidates from the Octet platform.

FIGS. 13A-13F show example results from the development of arginine recognizers. FIG. 13A shows polarization response for PS621 variants binding to RA, KA, and HA peptides (top chart) and binding affinities (Kd) determined by polarization for select PS621 variants for RA and HA at 20° C. (bottom table). FIG. 13B shows Kd determination titration curves for RA binding by PS621, PS691, and PS1122. FIGS. 13C-13D show on-chip recognition of RA dipeptide with PS1122 using QP304-RAIFAG in a recognition on-chip assay. FIGS. 13E-13F show an example of multiplexed dynamic chip analysis of PS1122 to demonstrate its enhanced range of arginine tripeptide coverage. Three peptides (RLQFQALMAADDD (SEQ ID NO: 1139), LAQRQAFDAADDD (SEQ ID NO: 1140), FAQLQARFAADDD (SEQ ID NO: 1141)) containing different RXA motifs were evaluated simultaneously in one sequencing run.

FIGS. 14A-14C show example results from computational modeling of PS961 showing N41D enhances electrostatic interactions with the N-terminal amino group of a polypeptide. FIG. 14A shows the amino acid triad D73, D43, and N41 (left panel) or D41 (right panel) bind to the N-terminal amino group of an AAA-tripeptide (sticks, top middle) via hydrogen bonds (dashed lines). FIG. 14B shows the PS961 binding pocket is more negatively charged than that of PS557, thus increasing the likelihood of a protein-peptide interaction. FIG. 14C shows the average fa_elec energy term of the peptide N-terminus over 50 representative structures from molecular dynamics simulations is lower (more favorable) in PS961 than PS557.

FIG. 15 shows example results from computational modeling of PS961 showing V72M increases hydrophobic interactions with the peptide and further packs the protein core. The longer methionine side chain can pack closely against neighboring residues (Van der Waals radii shown as spheres) and interact with the side chain of the peptide N-terminus (sticks and spheres, top middle) more so than its valine precursor.

FIG. 16 shows example results from computational modeling of PS961 showing L68M further optimizes protein core packing. Similar to V72M, the longer methionine side chain extends further into a non-optimal protein cavity than leucine (Van der Waals radii shown as spheres), providing stabilizing hydrophobic interactions.

FIGS. 17A-17B show example results from computational modeling of PS961 showing Y100R mutation neutralizes and increases the charge of a small negative surface pocket away from the binding pocket, which could compete with the intended recognition pocket.

FIGS. 18A-18B show example results from computation modeling of PS961 showing Y100R allows for a unique loop structure that is more amenable to antepenultimate interactions. FIG. 18A shows R100 stabilizes an extensive hydrogen bond network between the antepenultimate main chain (AP; sticks, left), R106, and other members of the loop. FIG. 18B shows the average occupancy of the R100:R106 and R106:AP hydrogen bonds depicted in FIG. 18A are higher in PS961 simulations compared to PS557 (50 ns trajectories, n=3).

FIGS. 19A-19C show secondary structure, sequence, and binding pocket properties of PS961. FIG. 19A shows a classification of the protein into secondary structure groups. FIG. 19B shows a Poisson Boltzmann electrostatic potential surface map of the binding pocket with residues labeled that form the binding pocket, with corresponding pocket properties listed. FIG. 19C shows the sequence of residues 32-116 of natural parent protein (PS557 (SEQ ID NO: 1)) and residues 32-116 of engineered variant (PS961 (SEQ ID NO: 314)) highlighting mutations, pocket positions and secondary structure assignment per position.

FIGS. 19D-19S show example results from crystal structure analysis of PS961 in complex with N-terminal methionine peptide (FIG. 19D-19K) or N-terminal alanine peptide (FIGS. 19L-19S). FIG. 19D shows crystals of PS961:MAKL complex. FIG. 19E shows the crystal structure of recognizer PS961 (surface) in complex with target peptide MAKL (SEQ ID NO: 1047) (sticks). FIGS. 19F-19K show how PS961 binds the target peptide MAKL (SEQ ID NO: 1047). FIG. 19L shows the crystal structure of recognizer PS961 (cartoon) in complex with target peptide AAKL (SEQ ID NO: 1048) (sticks). FIG. 19M shows a backbone superimposition of PS961 bound to MAKL (SEQ ID NO: 1047) and to AAKL (SEQ ID NO: 1048). FIG. 19N shows displacement of the Asp42 sidechain in the recognizer when comparing binding to AAKL (SEQ ID NO: 1048) and to MAKL (SEQ ID NO: 1047).

FIG. 19O shows a comparison of the AAKL (SEQ ID NO: 1048) and MAKL (SEQ ID NO: 1047) peptides when bound to PS961. FIG. 19P shows a comparison of the interaction of the AAKL (SEQ ID NO: 1048) (left panel) and MAKL (SEQ ID NO: 1047) (right panel) peptides with residues in PS961. FIG. 19Q shows reorientation of residue Asp12 in PS961 to bind either the MAKL (SEQ ID NO: 1047) (with a water molecule as the mediator) or the AAKL (SEQ ID NO: 1048) peptide. FIG. 19R shows reorientation of residue Asp42 in PS961 to bind either the MAKL (SEQ ID NO: 1047) or the AAKL (SEQ ID NO: 1048) peptide. FIG. 19S shows a superimposition of the AAKL (SEQ ID NO: 1048) and MAKL (SEQ ID NO: 1047) peptides showing the 1800 flip of the LYS sidechain in the third position (left), and different orientations of the Lys3 sidechain in the MAKL (SEQ ID NO: 1047) and AAKL (SEQ ID NO: 1048) peptides when bound to PS961 (right).

FIG. 20A shows example results from computational modeling of PS1122 showing PS621 crystal structure electrostatic surface with modeled mutations. Right-Top: E70T mutation creating a new hydrogen bond with the N-terminal arginine sidechain. Right-Middle: 163E mutation creating a new hydrogen bond with the amino terminus. Right-Bottom: T47 L mutation was found to be a generalist mutation across binding selections and likely improves the stability of the protein via stabilizing a helical turn near the metal binding sites.

FIGS. 20B-20F show example results from crystal structure analysis of PS1122 in complex with N-terminal arginine peptide (RAKL (SEQ ID NO: 1049)). FIG. 20B shows crystals of PS1122:RAKL complex. FIG. 20C shows the crystal structure of recognizer PS1122 in complex with target peptide RAKL (SEQ ID NO: 1049) (sticks). FIG. 20D shows that a portion of PS1122 is different than the analogous portion observed in PS621 (the PS1122 predecessor). FIG. 20E shows the NH3 group of the first amino acid of Arg-1 of the bound peptide interact with amino acids Glu-63, Asp-65, and a water molecule that is held in place by Glu-63 (interactions are depicted as dashed lines). FIG. 20F shows that the side chains of Glu-63 and Thr-70 of PS1122 both make interactions with the Arg-1 of the bound peptide.

FIGS. 21A-21C show secondary structure, sequence, and binding pocket properties of PS1122. FIG. 21A shows a classification of protein into secondary structure groups. FIG. 21B shows Poisson Boltzmann electrostatic potential surface map of binding pocket with residues labeled that form the binding pocket, with corresponding pocket properties listed. FIG. 21C shows the sequence residues 1-82 of natural parent protein (PS621 (SEQ ID NO: 2)) and residues 1-82 of engineered variant (PS1122 (SEQ ID NO: 468)) highlighting mutations, pocket positions and secondary structure assignment per position.

FIG. 22 shows an alphafold model of PS1259 with network of hydrogen bonds enabled by two mutations: C25S (below N-terminal Glutamine, shown in sticks) and H78Q (above N-terminal Glutamine) shown in sticks.

FIGS. 23A-23C show secondary structure, sequence, and binding pocket properties of PS1259. FIG. 23A shows a classification of the protein into secondary structure groups. FIG. 23B shows Poisson Boltzmann electrostatic potential surface map of binding pocket with residues labeled that form the binding pocket, with corresponding pocket properties listed. FIG. 23C shows the sequence of natural parent protein (Ntaq1(sf) (SEQ ID NO: 3)) and engineered variant PS1259 (SEQ ID NO: 605) highlighting mutations, pocket positions and secondary structure assignment per position.

FIGS. 24A-24G show example results showing direct identification of arginine PTMs. FIG. 24A shows different arginine PTMs (YRELRLLK (SEQ ID NO: 1077), YR_{ADMA}ELRLLK (SEQ ID NO: 1078) and YR_{SDMA}ELRLLK (SEQ ID NO: 1079)), including symmetric dimethylarginine (SDMA), asymmetric dimethylarginine (ADMA), and citrullinated arginine. FIG. 24B shows an exemplary workflow for collecting samples, preparing libraries of digested peptides, loading on a chip, and conducting on-chip sequencing and data analysis. FIGS. 24C-24E show sequencing data demonstrating that kinetic signatures distinguish peptides containing arginine, ADMA, and SDMA. FIG. 24C shows example protein sequencing traces for three synthetic P38MAPKα-derived peptides containing arginine, ADMA, or SDMA at position 2. Full length peptide sequences are indicated for each example trace. FIG. 24D shows the distribution of recognition segment (RS) mean pulse duration (PD) for RSs corresponding to the initial 4-residue sequence of each peptide: YREL (SEQ ID NO: 1050) (left), YR_{ADMA}EL (SEQ ID NO: 1051) (middle), and YR_{SDMA}EL (SEQ ID NO: 1052) (right). Median values are indicated for each distribution. FIG. 24E shows interpulse duration (IPD) for arginine v. ADMA detection by PS621. FIGS. 24F-24G show sequencing data demonstrating that kinetic signatures distinguish peptides containing arginine and citrulline. FIG. 24F shows example protein sequencing traces for two synthetic peptides containing arginine or citrulline at position 2: peptide sequence LRLAFAYPDDDK (SEQ ID NO: 1053) (QP707) and citrullinated peptide sequence LRcitLAFAYPDDDK (SEQ ID NO: 1054) (QP789). Full length peptide sequences are indicated for each example trace. FIG. 24G shows the distribution of RS mean PD for RSs corresponding to the initial 5-residue sequence of each peptide: LRLAF (SEQ ID NO: 1055) (left) and LCitLAF (SEQ ID NO: 1056) (right). Median values are indicated for each distribution.

FIGS. 25A-25C show example results showing identification of threonine PTM. FIGS. 25A-25B show results from sequencing reactions using the recognizers PS691, PS610, and PS961 for the peptides: RLTFIAYPDDD (SEQ ID NO: 1057) (FIG. 25A); and RLpTFIAYPDDD (SEQ ID NO: 1058), where pT is phosphothreonine (FIG. 25B). FIG. 25C shows recognition segment (RS) durations for leucine recognition in the sequencing reactions of FIG. 25A (left panel) and 25B (right panel).

FIGS. 26A-26B show example results showing identification of tyrosine PTM in sequencing reactions using the recognizers PS691, PS610, and PS961 for the peptides: RLYFIAYPDDD (SEQ ID NO: 1059) (FIG. 26A); and RLpYFIAYPDDD (SEQ ID NO: 1060), where pY is phosphotyrosine (FIG. 26B).

FIGS. 27A-27B show example results showing identification of lysine PTM in sequencing reactions using the recognizers PS691, PS610, PS961, and PS1165 for the peptides: RLYFKAYPDDD (SEQ ID NO: 1061) (FIG. 27A); and RLK{acetyl}FIAYPDDD (SEQ ID NO: 1062), where K{acetyl} is a acetylated lysine (FIG. 27B).

FIG. 28A-FIG. 28G illustrates aspects of an example application of the technology to identification of β-amyloid variants. FIG. 28A illustrates an example of a β-amyloid variant. FIG. 28B illustrates an example workflow for β-amyloid variant detection. FIG. 28C-FIG. 28G illustrate examples of pulse patterns of β-amyloid Wild Type LVFFAE (SEQ ID NO: 1063) versus variants (LVFFAK (SEQ ID NO: 1064), LVFFGK (SEQ ID NO: 1065), LVFFAG (SEQ ID NO: 1066), LVPFAE (SEQ ID NO: 1067)).

FIG. 29 shows an example schematic of a pixel of an integrated device.

FIGS. 30A-30I show example results from computational modeling (FIGS. 30A-30F) and the structures of model peptides (RLAF (SEQ ID NO: 1142) (FIG. 30C), ADMA-LAF (SEQ ID NO: 1143) (FIG. 30D), SDMA-LAF (SEQ ID NO: 1144) (FIG. 30E) and Cit-LAF (SEQ ID NO: 1145) (FIG. 30F)) evaluated with PS621 and PS1122 (FIGS. 30G-30I).

FIG. 31 shows amino acid frequency across the human proteome.

FIG. 32A shows high-throughput expression, purification and conjugation with streptavidin for hNTAQ protein homolog variants.

FIGS. 32B-32E show example traces for sequencing reactions performed with six recognizers, including the glutamate recognizer PS1875.

FIGS. 32F-32G show expression and purification of bis-biotinylated PS2132 in 2 L scale.

FIGS. 32H-32I show example results from size exclusion chromatography (SEC) of PS2132 labeled with streptavidin-linked long-lifetime BODIPY dye.

FIG. 32J shows example results from Quality Control SDS PAGE gel analysis of PS2132 labeled with long-lifetime BODIPY dye pre- and post-SEC column purification.

FIGS. 33A-33F show example results from on-chip recognition of E by PS1875 (FIGS. 33A-33C) and PS2132 (FIGS. 33D-33F) labeled with long-lifetime BODIPY dye.

FIGS. 34A-34F show example results from on-chip recognition of E by PS1875 (FIGS. 34A-34C) and PS2121 (FIGS. 34D-34F) labeled with long-lifetime BODIPY dye.

FIGS. 35A-35F show example results from on-chip recognition of E by PS1875 (FIGS. 35A-35C) and PS2123 (FIGS. 35D-35F) labeled with long-lifetime BODIPY dye.

FIG. 36 shows pulse duration (top) and interpulse duration (bottom) of RSs corresponding to E recognition in aligned reads for sequencing runs performed using QP1165 (EIAFLKQRVWK (SEQ ID NO: 1084) peptide with a mixture of 6 recognizers containing either PS1875, PS2132, PS2121, or PS2123 as the recognizer for glutamate (E).

FIGS. 37A-37C show example results from sequencing runs of a CDNF library performed using reagent A containing a mixture of 5 recognizers (FIG. 37A) or reagent A combined with the E recognizer PS2132 (FIG. 37B). FIG. 37C depicts the sequence of CDNF (SEQ ID NO: 1146) and example traces for 3 peptides: EFLNRFYK (SEQ ID NO: 1068), ELISFCLDTK (SEQ ID NO: 1069), and ENRLCYYLGATK (SEQ ID NO: 1070).

FIGS. 38A-38C show example results from sequencing runs performed on GFAP peptide library with (FIG. 38A) or without (FIG. 38B) recognizer for glutamate (E) PS2132 in combination with five recognizers. FIG. 38C depicts the sequence of GFAP (SEQ ID NO: 1147) and example traces for 2 peptides: DEMARHLQEYQDLLNVK (SEQ ID NO: 1071) and LALDIEIATYRK (SEQ ID NO: 1072).

FIGS. 39A-39B show example results from computational modeling of PS2132. FIG. 39A depicts the binding pocket of PS2132 in complex with N-terminal glutamate peptide. FIG. 39B depicts modeling of surface charge for PS1259 and PS2132.

### DETAILED DESCRIPTION

Aspects of the disclosure relate to compositions and methods for determining chemical characteristics of a polypeptide based on single-molecule binding interactions between the polypeptide and one or more reagents described herein. In some embodiments, the disclosure provides an approach for polypeptide structure analysis based on kinetic information derived from single-molecule binding interactions between a polypeptide and one or more amino acid recognizers described herein.

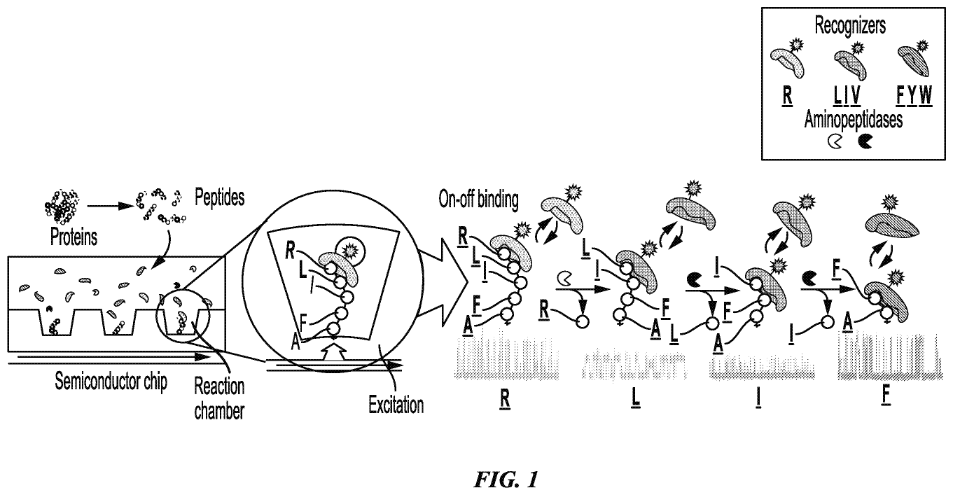

FIG. 1 shows an example of a dynamic peptide sequencing reaction in which individual on-off binding events give rise to signal pulses of a signal output. As shown at left, a protein sample may be fragmented into peptides, which are immobilized in reaction chambers of an array, where the immobilized peptides are exposed to one or more amino acid recognizers and one or more cleaving reagents (e.g., aminopeptidases). As shown at right, an amino acid recognizer reversibly binds a terminal end of the peptide, and a pulse in signal output is produced while the recognizer is bound to the peptide.

As the on-off binding of recognizers generally occurs at a faster rate than amino acid cleavage, the binding events preceding each cleavage event give rise to a series of changes in the signal (e.g., signal pulses), which can be used to determine structural information about amino acids at or near the terminal end of the peptide. Compositions and methods for performing dynamic polypeptide sequencing and analyzing data obtained therefrom are described more fully in PCT International Publication No. WO2020102741A1, filed Nov. 15, 2019, and PCT International Publication No. WO2021236983A2, filed May 20, 2021, each of which is incorporated by reference in its entirety.

In some aspects, the disclosure provides amino acid recognizers with improved binding properties, which allow for more structural information to be obtained from polypeptides based on the kinetics of on-off binding between recognizer and polypeptide. In some embodiments, an amino acid recognizer comprises an amino acid binding protein with an engineered binding pocket having one or more modifications relative to a homologous protein. In some embodiments, the modified binding pocket increases the number of interactions (e.g., hydrogen bonding interactions, van der Waals interactions) formed between the binding pocket and an amino acid ligand as compared to an unmodified binding pocket of the homologous protein. In some embodiments, the modified binding pocket increases the number of types of amino acid ligands capable of being detectably bound as compared to an unmodified binding pocket of the homologous protein. In some embodiments, the modified binding pocket improves the kinetics of binding (e.g., K_{D}, k_{off}, kₒₙ) toward one or more types of amino acid ligands, which advantageously increases the amount of, or confidence in, structural information that may be derived from polypeptide analysis as described herein.

### I. Amino Acid Recognizers

In some aspects, the disclosure provides an amino acid recognizer comprising an amino acid binding protein having an amino acid sequence selected from Table 1. Table 1 herein provides a list of example sequences of amino acid binding proteins. It should be appreciated that these sequences and other examples described herein are meant to be non-limiting, and amino acid recognizers in accordance with the disclosure can include any homologs, variants, or fragments thereof minimally containing domains or subdomains responsible for amino acid recognition.

In some embodiments, the disclosure provides an amino acid binding protein having an amino acid sequence that is at least 80% identical to an amino acid sequence selected from Table 1. In some embodiments, an amino acid binding protein has at least 40%, at least 50%, at least 60%, at least 70%, at least 80%, at least 85%, at least 90%, at least 95%, at least 98%, or higher, amino acid sequence identity to an amino acid sequence selected from Table 1. In some embodiments, an amino acid binding protein has 25-50%, 50-60%, 60-70%, 70-80%, 80-90%, 90-95%, 95-99%, 40-100%, 50-100%, 60-100%, 70-100%, 80-100%, 90-100%, or 95-100% amino acid sequence identity to an amino acid sequence selected from Table 1.

For the purposes of comparing two or more amino acid sequences, the percentage of “sequence identity” between a first amino acid sequence and a second amino acid sequence (also referred to herein as “amino acid identity”) may be calculated by: dividing [the number of amino acid residues in the first amino acid sequence that are identical to the amino acid residues at the corresponding positions in the second amino acid sequence] by [the total number of amino acid residues in the first amino acid sequence] and multiplying by [100], in which each deletion, insertion, substitution or addition of an amino acid residue in the second amino acid sequence compared to the first amino acid sequence is considered as a difference at a single amino acid residue (position). Alternatively, the degree of sequence identity between two amino acid sequences may be calculated using a known computer algorithm (e.g., by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman. Proc. Natl. Acad. Sci. USA (1998) 85:2444, or by computerized implementations of algorithms available as Blast, Clustal Omega, or other sequence alignment algorithms) and, for example, using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Additionally, or alternatively, two or more sequences may be assessed for the identity between the sequences. The terms “identical” or percent “identity” in the context of two or more nucleotide or amino acid sequences, refer to two or more sequences or subsequences that are the same. Two sequences are “substantially identical” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8%, or 99.9% identical) over a specified region or over the entire sequence, when compared and aligned for maximum correspondence over a comparison window, or designated region as measured using one of the above sequence comparison algorithms or by manual alignment and visual inspection. Optionally, the identity exists over a region that is at least about 25, 50, 75, or 100 amino acids in length, or over a region that is 100 to 150, 150 to 200, 100 to 200, or 200 or more, amino acids in length.

Additionally, or alternatively, two or more sequences may be assessed for the alignment between the sequences. The terms “alignment” or percent “alignment” in the context of two or more nucleotide or amino acid sequences, refer to two or more sequences or subsequences that are the same. Two sequences are “substantially aligned” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using one of the above sequence comparison algorithms or by manual alignment and visual inspection. Optionally, the alignment exists over a region that is at least about 25, 50, 75, or 100 amino acids in length, or over a region that is 100 to 150, 150 to 200, 100 to 200, or 200 or more amino acids in length.

In some embodiments, an amino acid recognizer of the disclosure comprises a modified amino acid binding protein and includes one or more amino acid deletions, additions, or mutations relative to a sequence set forth in Table 1. In some embodiments, a modified amino acid binding protein includes a deletion, addition, or mutation of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acids (which may or may not be consecutive amino acids) relative to a sequence set forth in Table 1.

### A. ClpS-Homologous Recognizers

In some embodiments, an amino acid recognizer of the disclosure binds an amino acid ligand (e.g., a polypeptide) comprising an N-terminal amino acid selected from leucine, isoleucine, valine, methionine, alanine, or a modified variant thereof (e.g., a post-translationally modified variant thereof, an oxidized variant thereof). In some embodiments, the amino acid recognizer comprises an amino acid binding protein derived from a ClpS protein, such as Planctomycetia bacterium ClpS protein. For example, in some embodiments, the amino acid binding protein is an engineered variant comprising one or more modifications relative to SEQ ID NO: 1 as described herein.

In some embodiments, the amino acid binding protein binds N-terminal leucine with a dissociation constant (K_{D}) of less than 2,000 nM, less than 1,500 nM, less than 1,000 nM, less than 750 nM, less than 500 nM, less than 250 nM, less than 150 nM, less than 100 nM, less than 80 nM, 10-2,000 nM, 25-1,000 nM, 50-500 nM, 10-150 nM, 25-75 nM, or 50-60 nM. In some embodiments, the amino acid binding protein binds N-terminal isoleucine with a K_{D} of less than 2,000 nM, less than 1,500 nM, less than 1,000 nM, less than 750 nM, less than 500 nM, less than 250 nM, less than 150 nM, less than 100 nM, less than 80 nM, 10-2,000 nM, 25-1,000 nM, 50-500 nM, 10-150 nM, 30-80 nM, or 60-75 nM. In some embodiments, the amino acid binding protein binds N-terminal valine with a K_{D} of less than 2,000 nM, less than 1,500 nM, less than 1,000 nM, less than 750 nM, less than 500 nM, less than 300 nM, less than 250 nM, less than 200 nM, 10-2,000 nM, 25-1,000 nM, 50-500 nM, 50-300 nM, or 100-200 nM.

In some embodiments, the amino acid binding protein binds one or more types of N-terminal amino acids (e.g., leucine, isoleucine, valine, methionine, and/or alanine), where each type of binding interaction is characterized by a dissociation rate (k_{off}) of at least 0.1 s⁻¹.

In some embodiments, the dissociation rate is between about 0.1 s⁻¹ and about 1,000 s⁻¹ (e.g., between about 0.5 s⁻¹ and about 500 s⁻¹, between about 0.1 s⁻¹ and about 100 s⁻¹, between about 1 s⁻¹ and about 100 s⁻¹, or between about 0.5 s⁻¹ and about 50 s⁻¹). In some embodiments, the dissociation rate is between about 0.5 s⁻¹ and about 20 s⁻¹. In some embodiments, the dissociation rate is between about 2 s⁻¹ and about 20 s⁻¹. In some embodiments, the dissociation rate is between about 0.5 s⁻¹ and about 2 s⁻¹.

In some aspects, the disclosure provides a recombinant or synthetic amino acid binding protein having an amino acid sequence that is at least 40%, at least 50%, at least 60%, at least 70%, at least 80%, at least 85%, at least 90%, at least 95%, at least 98%, or 100% identical to a sequence selected from any one of PS635-645, PS731-732, PS759-766, PS769, PS795-870, PS896-912, PS918-1043, PS1048-1100, PS1124-1137, PS1141-1161, PS1175-1199, PS1203-1217, PS1222-1245, PS1277-1305, PS1321-1350, and PS1425-1448 (SEQ ID NOs: 22-27, 87-88, 115-122, 125, 151-226, 249-265, 271-390, 395-446, 470-483, 487-507, 521-545, 549-563, 568-591, 622-650, 664-693, and 768-791). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS635-645, PS731-732, PS759-766, PS769, PS795-870, PS896-912, PS918-1043, PS1048-1100, PS1124-1137, PS1141-1161, PS1175-1199, PS1203-1217, PS1222-1245, PS1277-1305, PS1321-1350, and PS1425-1448 (SEQ ID NOs: 22-27, 87-88, 115-122, 125, 151-226, 249-265, 271-390, 395-446, 470-483, 487-507, 521-545, 549-563, 568-591, 622-650, 664-693, and 768-791). In some embodiments, the amino acid sequence is at least 40%, at least 50%, at least 60%, at least 70%, at least 80%, at least 85%, at least 90%, at least 95%, at least 98%, or 100% identical to PS961 (SEQ ID NO: 314). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to PS961 (SEQ ID NO: 314).

In some aspects, the disclosure provides a recombinant or synthetic amino acid binding protein having an amino acid sequence that is at least 80% identical (e.g., at least 85%, at least 90%, at least 95%, at least 98%, 80-98%, 80-95%, 80-90%, 85-95%, or 90-98% identical) to SEQ ID NO: 1, where the amino acid sequence comprises an amino acid substitution at one or more positions corresponding to E22, R31, L39, N41, D42, D43, D44, H45, T46, Y47, V50, Q55, P62, E63, L68, A69, V72, D73, Q75, Y100, and M111 of SEQ ID NO: 1.

In some embodiments, the amino acid sequence comprises an amino acid substitution at a position corresponding to N41, and at one or more positions corresponding to E22, R31, L39, D42, H45, V50, Q55, P62, E63, L68, V72, Q75, Y100, and M11 L. In some embodiments, the amino acid sequence comprises an amino acid substitution at a position corresponding to N41 and at one or more positions corresponding to Q55, E63, L68, V72, and Y100. In some embodiments, the amino acid sequence comprises an amino acid substitution selected from E22V, R31H, L39M, N41D, D42 L, D42P, H45C, H45F, V50A, V50F, V50Y, Q55H, Q55R, P62R, E63A, E63G, E63K, E63S, L68M, V72M, Q75 L, Y100R, M111A, and M111S. In some embodiments, the amino acid substitution is selected from N41D, Q55R, E63S, L68M, V72M, and Y100R.

In some aspects, the disclosure provides a recombinant or synthetic amino acid binding protein comprising a structure of Formula (I) or a structural equivalent thereof:

(β1-α1-α2-β2-α3-β3 (I),

wherein: each of β1, β2, and β3 is a beta-strand; each of α1, α2, and α3 is an alpha-helix; each instance of “-” is a loop; and at least a portion of each of α1, α2, the loop between β1 and α1, and the loop between α3 and β3 form a binding pocket for an amino acid ligand.

In some embodiments, the binding pocket comprises one or more of the following: (i) a volume of approximately 170 Å³, (ii) an electrostatic potential of −3.0 RTe_{c}⁻¹ or less, (iii) negatively charged side-chains in at least 35% of amino acids that form the binding pocket, (iv) a plurality of hydrogen bond acceptors configured to form one or more hydrogen bonds (e.g., at least two, at least three, at least four, or at least five hydrogen bonds) in the presence of the amino acid ligand, and (v) a plurality of van der Waals contact positions configured to form van der Waals interactions in the presence of the amino acid ligand. In some embodiments, the binding pocket comprises one, two, three, or four of (i), (ii), (iii), (iv), and (v). In some embodiments, the binding pocket comprises (i), (ii), (iii), (iv), and (v).

In some embodiments, the binding pocket comprises a volume of approximately 170 Å³. In some embodiments, the volume of the binding pocket is at least 150 Å³, at least 160 Å³, at least 170 Å³, at least 180 Å³, or at least 190 Å³. In some embodiments, the volume of the binding pocket is no more than 190 Å³, no more than 180 Å³, no more than 170 Å³, no more than 160 Å³, or no more than 150 Å³. In some embodiments, the volume of the binding pocket is in a range from 150 Å³ to 170 Å³, 150 Å³ to 180 Å³, 150 Å³ to 190 Å³, 160 Å³ to 180 Å³, 160 Å³ to 190 Å³, 170 Å³ to 190 Å³, or 180 Å³ to 190 Å³. Methods for determining volume of a binding pocket are known in the art and will be apparent to a skilled person in view of the present disclosure. For example, in some embodiments, the volume of the binding pocket is determined using software configured to measure geometric and topological properties of proteins. In some embodiments, the software may scan a protein surface using a specified probe radius to measure the volume of any cavities that directly overlap with a binding site or indirectly overlap with the binding site through adjacent cavities within van der Waals contact of one another. A non-limiting example of a suitable probe radius is a solvent probe radius (e.g., about 1.4 Å). A non-limiting example of suitable software is Computed Atlas of Surface Topography of proteins (CASTp). See, e.g., W. Tian et al., CASTp 3.0: Computed atlas of surface topography of proteins. Nucleic Acids Res. 46, W363-W367 (2018), the relevant content of which is incorporated herein by reference.

In some embodiments, the binding pocket has an electrostatic potential of −3.0 RTe_{c}-′ or less. In some embodiments, the electrostatic potential of the binding pocket is at least −4 RTe_{c}⁻¹, at least −3 RTe_{c}⁻¹, or at least −2 RTe_{c}⁻¹. In some embodiments, the electrostatic potential of the binding pocket is −2 RTe_{c}⁻¹ or less, −3 RTe_{c}⁻¹ or less, or −4 RTe_{c}⁻¹ or less. In some embodiments, the electrostatic potential of the binding pocket is in a range from −2 RTe_{c}⁻¹ to −3 RTe_{c}⁻¹, −2 RTe_{c}⁻¹ to −4 RTe_{c}⁻¹, or −3 RTe_{c}⁻¹ to −4 RTe_{c}⁻¹. Methods for determining electrostatic potential of a binding pocket are known in the art and will be apparent to a skilled person in view of the present disclosure. For example, in some embodiments, the Adaptive Poisson-Boltzmann Solver (APBS) tool in PyMOL (PyMOL Molecular Graphics System, Version 2.0 Schrödinger, LLC) may be used (e.g., with default parameters) to calculate the electrostatic surface potential of a binding pocket using pdb2pgr with the AMBER forcefield to assign protonation states. See, e.g., T. J. Dolinsky et al., PDB2PQR: An automated pipeline for the setup of Poisson-Boltzmann electrostatics calculations, Nucleic Acids Res., 32:W665-7 (2004); M. G. Lerner et al., APBS plugin for PyMOL—Version 2.4 (University of Michigan, Ann Arbor, M I, 2006); J. W. Ponder et al., Force fields for protein simulations, Adv. Protein Chem. 66: 27-85 (2003). In some cases, this solvent-accessible surface area (SASA) may be considered to be accessible by the peptide ligands.

In some embodiments, the binding pocket comprises a plurality of hydrogen bond acceptors configured to form one or more hydrogen bonds in the presence of the amino acid ligand. In some embodiments, the binding pocket forms at least two (e.g., at least three, at least four, at least five, 2-10, 4-10, 5-15, 5-10) hydrogen bonds with an amino acid ligand. Methods for determining hydrogen bond interactions between a binding pocket and a ligand are known in the art and will be apparent to a skilled person in view of the present disclosure. For example, in some embodiments, hydrogen bond interactions are determined by computational modeling as described in the Examples herein (e.g., using atomic coordinates for protein-ligand structural data derived from X-ray crystallography, or through computational modeling to predict the protein-ligand three-dimensional structure).

In some embodiments, the plurality of hydrogen bond acceptors include one or more atoms of a side-chain of an amino acid residue in the binding pocket. For example, in some embodiments, the binding pocket comprises at least one negatively charged amino acid side-chain (e.g., aspartate, glutamate) that forms a bifurcated hydrogen bond with the amino acid ligand (e.g., an N-terminal amino acid of a polypeptide). In some embodiments, at least four hydrogen bonds are formed between the amino acid ligand (e.g., an N-terminal amino acid of a polypeptide) and amino acid side-chains in the binding pocket. In some embodiments, the plurality of hydrogen bond acceptors include one or more atoms of the polypeptide backbone (e.g., backbone carbonyl) in the binding pocket.

In some embodiments, the binding pocket forms one or more hydrogen bonds with a side chain of an amino acid ligand. For example, in some embodiments, the binding pocket forms one or more hydrogen bonds with a side chain of a terminal amino acid of an amino acid ligand (e.g., a polypeptide). In some embodiments, the binding pocket forms one or more hydrogen bonds with the polypeptide backbone of an amino acid ligand (e.g., a polypeptide). In some embodiments, the binding pocket forms one or more hydrogen bonds with a terminal amino acid and one or more amino acids contiguous to the terminal amino acid in a polypeptide (e.g., amino acids at position 1 and at positions 2, 3, 4, and/or 5 relative to the polypeptide terminus).

In some embodiments, the binding pocket comprises a plurality of van der Waals contact positions configured to form van der Waals interactions in the presence of the amino acid ligand. Methods for determining van der Waals interactions between a binding pocket and a ligand are known in the art and will be apparent to a skilled person in view of the present disclosure. For example, in some embodiments, van der Waals interactions are determined by computational modeling as described in the Examples herein (e.g., using atomic coordinates for protein-ligand structural data derived from X-ray crystallography, or through computational modeling to predict the protein-ligand three-dimensional structure).

In some embodiments, the van der Waals contact positions comprise a plurality of atoms (e.g., 2-30, 5-25, 10-20, 2-10, 5-10) configured to form hydrophobic interactions with an amino acid ligand. In some embodiments, one or more atoms of the plurality of atoms are non-polar atoms. In some embodiments, the van der Waals contact positions are formed by a methionine side chain in the binding pocket.

In some embodiments, the amino acid ligand is a polypeptide comprising at least three amino acids. In some embodiments, the amino acid ligand comprises an N-terminal amino acid of a polypeptide. In some embodiments, the N-terminal amino acid is selected from leucine, isoleucine, valine, methionine, and alanine. In some embodiments, the amino acid binding protein is at least 50 amino acids in length, at least 75 amino acids in length, at least 100 amino acids in length, 50-250 amino acids in length, 50-150 amino acids in length, or 100-200 amino acids in length.

In some embodiments, the loop between β1 and α1 comprises three or more negatively charged amino acids. In some embodiments, the loop between β1 and α1 comprises four or more negatively charged amino acids. In some embodiments, at least two negatively charged amino acids of the loop between β1 and α1 form a hydrogen bond with the amino acid ligand. In some embodiments, at least one negatively charged amino acid of the loop between β1 and α1 forms a bifurcated hydrogen bond with the amino acid ligand. In some embodiments, the negatively charged amino acids are selected from aspartate and glutamate.

In some embodiments, β1-α1 comprises an amino acid sequence that is at least 80% identical to a sequence of amino acids 35-58 of SEQ ID NO: 1. In some embodiments, the binding pocket is formed by amino acids at one or more positions corresponding to amino acids 41-47 and 50 of SEQ ID NO: 1. In some embodiments, the amino acid sequence comprises an amino acid substitution at one or more positions corresponding to L39, N41, D42, H45, V50, and Q55 of SEQ ID NO: 1. In some embodiments, at least one amino acid substitution is at a position corresponding to N41 of SEQ ID NO: 1. In some embodiments, the amino acid substitution is selected from N41D and Q55R.

In some embodiments, α2 comprises an amino acid sequence that is at least 80% identical to a sequence of amino acids 62-73 of SEQ ID NO: 1. In some embodiments, the binding pocket is formed by amino acids at one or more positions corresponding to amino acids 69, 72, and 73 of SEQ ID NO: 1. In some embodiments, the amino acid sequence comprises an amino acid substitution at one or more positions corresponding to P62, E63, L68, and V72 of SEQ ID NO: 1. In some embodiments, at least one amino acid substitution is at a position corresponding to V72 of SEQ ID NO: 1. In some embodiments, the amino acid substitution is selected from E63S, L68M, and V72M.

In some embodiments, the loop between α3 and β3 comprises an amino acid sequence that is at least 80% identical to a sequence of amino acids 99-112 of SEQ ID NO: 1. In some embodiments, the binding pocket is formed by an amino acid at a position corresponding to amino acid 111 of SEQ ID NO: 1. In some embodiments, the amino acid sequence comprises an amino acid substitution at one or more positions corresponding to Y100 and M111 of SEQ ID NO: 1. In some embodiments, the amino acid substitution is Y100R.

In some embodiments, the structural equivalent is a structure having a root-mean-square difference of no more than 5.0 Å where at least 80% of secondary structure alpha-carbon atoms are aligned with the structure of Formula (I). In some embodiments, the structural equivalent is a structure having a root-mean-square difference of no more than 4.0 Å, no more than 3.0 Å, no more than 2.0 Å, or no more than 1.0 Å where at least 80% of secondary structure alpha-carbon atoms are aligned with the structure of Formula (I).

Methods for identifying a structural equivalent to the structure of Formula (I) are known in the art and will be apparent to a skilled person in view of the present disclosure. For example, in some embodiments, a structural equivalent to Formula (I) is a structure having a root-mean-square difference of no more than 5.0 Å where at least 80% of secondary structure alpha-carbon atoms are aligned with a three-dimensional protein structure having an amino acid sequence selected from any one of PS635-645, PS731-732, PS759-766, PS769, PS795-870, PS896-912, PS918-1043, PS1048-1100, PS1124-1137, PS1141-1161, PS1175-1199, PS1203-1217, PS1222-1245, PS1277-1305, PS1321-1350, and PS1425-1448 (SEQ ID NOs: 22-27, 87-88, 115-122, 125, 151-226, 249-265, 271-390, 395-446, 470-483, 487-507, 521-545, 549-563, 568-591, 622-650, 664-693, and 768-791). A protein structure comparison may be performed by determining a three-dimensional structure for a protein having an amino acid sequence selected from any one of PS635-645, PS731-732, PS759-766, PS769, PS795-870, PS896-912, PS918-1043, PS1048-1100, PS1124-1137, PS1141-1161, PS1175-1199, PS1203-1217, PS1222-1245, PS1277-1305, PS1321-1350, and PS1425-1448 (SEQ ID NOs: 22-27, 87-88, 115-122, 125, 151-226, 249-265, 271-390, 395-446, 470-483, 487-507, 521-545, 549-563, 568-591, 622-650, 664-693, and 768-791), and comparing the three-dimensional structure for the protein to a candidate protein structure to determine whether the candidate protein is a structural equivalent. A three-dimensional protein structure can be determined, for example, using atomic coordinates derived from X-ray crystallography, or through computational modeling to predict the three-dimensional structure of a protein having an amino acid sequence selected from any one of PS635-645, PS731-732, PS759-766, PS769, PS795-870, PS896-912, PS918-1043, PS1048-1100, PS1124-1137, PS1141-1161, PS1175-1199, PS1203-1217, PS1222-1245, PS1277-1305, PS1321-1350, and PS1425-1448 (SEQ ID NOs: 22-27, 87-88, 115-122, 125, 151-226, 249-265, 271-390, 395-446, 470-483, 487-507, 521-545, 549-563, 568-591, 622-650, 664-693, and 768-791). Methods for comparing protein structures and determining values for root-mean-square difference are known in the art (see, for example, Kufareva I, Abagyan R. Methods of protein structure comparison. Methods Mol Biol. 2012; 857:231-57).

### B. UBR-Homologous Recognizers

In some embodiments, an amino acid recognizer of the disclosure binds an amino acid ligand (e.g., a polypeptide) comprising an N-terminal amino acid selected from arginine or a modified variant thereof (e.g., a post-translationally modified variant thereof, an oxidized variant thereof). In some embodiments, the amino acid recognizer comprises an amino acid binding protein derived from a UBR protein, such as Kluyveromyces marxianus UBR protein. For example, in some embodiments, the amino acid binding protein is an engineered variant comprising one or more modifications relative to SEQ ID NO: 2 as described herein.

In some embodiments, the amino acid binding protein binds N-terminal arginine with a dissociation constant (K_{D}) of less than 2,000 nM, less than 1,500 nM, less than 1,000 nM, less than 750 nM, less than 500 nM, less than 400 nM, less than 200 nM, less than 100 nM, 10-2,000 nM, 25-1,000 nM, 50-500 nM, 10-400 nM, 25-75 nM, or 50-80 nM.

In some embodiments, the amino acid binding protein binds one or more types of N-terminal amino acids (e.g., arginine or a modified variant thereof), where each type of binding interaction is characterized by a dissociation rate (k_{off}) of at least 0.1 s⁻¹. In some embodiments, the dissociation rate is between about 0.1 s⁻¹ and about 1,000 s⁻¹ (e.g., between about 0.5 s⁻¹ and about 500 s⁻¹, between about 0.1 s⁻¹ and about 100 s⁻¹, between about 1 s⁻¹ and about 100 s⁻¹, or between about 0.5 s⁻¹ and about 50 s⁻¹). In some embodiments, the dissociation rate is between about 0.5 s⁻¹ and about 20 s⁻¹. In some embodiments, the dissociation rate is between about 2 s⁻¹ and about 20 s⁻¹. In some embodiments, the dissociation rate is between about 0.5 s⁻¹ and about 2 s⁻¹.

In some aspects, the disclosure provides a recombinant or synthetic amino acid binding protein having an amino acid sequence that is at least 40%, at least 50%, at least 60%, at least 70%, at least 80%, at least 85%, at least 90%, at least 95%, at least 98%, or 100% identical to a sequence selected from any one of PS1101-1122, PS1218-1221, and PS1351-1398 (SEQ ID NOs: 447-468, 564-567, and 694-741). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS1101-1122, PS1218-1221, and PS1351-1398 (SEQ ID NOs: 447-468, 564-567, and 694-741). In some embodiments, the amino acid sequence is at least 40%, at least 50%, at least 60%, at least 70%, at least 80%, at least 85%, at least 90%, at least 95%, at least 98%, or 100% identical to PS1122 (SEQ ID NO: 468) or PS1381 (SEQ ID NO: 724). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to PS1122 (SEQ ID NO: 468) or PS1381 (SEQ ID NO: 724).

In some aspects, the disclosure provides a recombinant or synthetic amino acid binding protein having an amino acid sequence that is at least 80% identical (e.g., at least 85%, at least 90%, at least 95%, at least 98%, 80-98%, 80-95%, 80-90%, 85-95%, or 90-98% identical) to SEQ ID NO: 2, where the amino acid sequence comprises an amino acid substitution at one or more positions corresponding to G19, K26, S29, F30, D31, D32, T33, C34, V35, T47, G48, T53, T54, T57, E58, F59, N61, 163, D65, D68, E70, A71, H74, and T75 of SEQ ID NO: 2.

In some embodiments, the amino acid sequence comprises an amino acid substitution at positions corresponding to 163 and E70, and at one or more positions corresponding to G19, K26, S29, D32, T47, G48, T53, T54, T57, E58, F59, N61, H74, and T75. In some embodiments, the amino acid sequence comprises an amino acid substitution at one or more positions corresponding to K26, D32, T47, 163, and E70. In some embodiments, the amino acid sequence comprises an amino acid substitution selected from G19R, K26R, S29Q, D32R, D32Y, T47K, T47 L, T47R, G48R, G48Y, T53V, T54K, T57K, T57R, E58K, F59R, N61K, 163E, E70S, E70T, H74K, and T75E. In some embodiments, the amino acid substitution is selected from T47 L, 163E, and E70T. In some embodiments, the amino acid substitution is selected from K26R and D32R.

In some aspects, the disclosure provides a recombinant or synthetic amino acid binding protein comprising a structure of Formula (II) or a structural equivalent thereof:

β1-α1-β2-α2-α3 (II),

wherein: each of β1 and β2 is a beta-strand; each of α1, α2, and α3 is an alpha-helix; each instance of “-” is a loop; and at least a portion of each of α2, the loop between β1 and α1, and the loop between β2 and α2 form a binding pocket for an amino acid ligand.

In some embodiments, the binding pocket comprises one or more of the following: (i) a volume of approximately 200 Å³, (ii) an electrostatic potential of −3.0 RTe_{c}⁻¹ or less, (iii) a plurality of hydrogen bond acceptors configured to form one or more hydrogen bonds in the presence of the amino acid ligand, and (iv) a plurality of van der Waals contact positions configured to form van der Waals interactions in the presence of the amino acid ligand. In some embodiments, the binding pocket comprises one, two, or three of (i), (ii), (iii), and (iv). In some embodiments, the binding pocket comprises (i), (ii), (iii), and (iv).

In some embodiments, the binding pocket comprises a volume of approximately 200 Å³. In some embodiments, the volume of the binding pocket is at least 180 Å³, at least 190 Å³, at least 200 Å³, at least 210 Å³, or at least 220 Å³. In some embodiments, the volume of the binding pocket is no more than 220 Å³, no more than 210 Å³, no more than 200 Å³, no more than 190 Å³, or no more than 180 Å³. In some embodiments, the volume of the binding pocket is in a range from 180 Å³ to 200 Å³, 180 Å³ to 210 Å³, 180 Å³ to 220 Å³, 190 Å³ to 210 Å³, 190 Å³ to 220 Å³, 200 Å³ to 220 Å³, or 210 Å³ to 220 Å³. Methods for determining volume of a binding pocket are known in the art and will be apparent to a skilled person in view of the present disclosure. For example, in some embodiments, the volume of the binding pocket is determined using software configured to measure geometric and topological properties of proteins. In some embodiments, the software may scan a protein surface using a specified probe radius to measure the volume of any cavities that directly overlap with a binding site or indirectly overlap with the binding site through adjacent cavities within van der Waals contact of one another. A non-limiting example of a suitable probe radius is a solvent probe radius (e.g., about 1.4 Å). A non-limiting example of suitable software is Computed Atlas of Surface Topography of proteins (CASTp). See, e.g., W. Tian et al., CASTp 3.0: Computed atlas of surface topography of proteins. Nucleic Acids Res. 46, W363-W367 (2018), the relevant content of which is incorporated herein by reference.

In some embodiments, the binding pocket has an electrostatic potential of −3.0 RTe_{c}⁻¹ or less. In some embodiments, the electrostatic potential of the binding pocket is at least −4 RTe_{c}⁻¹, at least −3 RTe_{c}⁻¹, or at least −2 RTe_{c}⁻¹. In some embodiments, the electrostatic potential of the binding pocket is −2 RTe_{c}⁻¹ or less, −3 RTe_{c}⁻¹ or less, or −4 RTe_{c}⁻¹ or less. In some embodiments, the electrostatic potential of the binding pocket is in a range from −2 RTe_{c}⁻¹ to −3 RTe_{c}⁻¹, −2 RTe_{c}⁻¹ to −4 RTe_{c}⁻¹, or −3 RTe_{c}⁻¹ to −4 RTe_{c}⁻¹. Methods for determining electrostatic potential of a binding pocket are known in the art and will be apparent to a skilled person in view of the present disclosure. For example, in some embodiments, the Adaptive Poisson-Boltzmann Solver (APBS) tool in PyMOL (PyMOL Molecular Graphics System, Version 2.0 Schrödinger, LLC) may be used (e.g., with default parameters) to calculate the electrostatic surface potential of a binding pocket using pdb2pgr with the AMBER forcefield to assign protonation states. See, e.g., T. J. Dolinsky et al., PDB2PQR: An automated pipeline for the setup of Poisson-Boltzmann electrostatics calculations, Nucleic Acids Res., 32:W665-7 (2004); M. G. Lerner et al., APBS plugin for PyMOL—Version 2.4 (University of Michigan, Ann Arbor, M I, 2006); J. W. Ponder et al., Force fields for protein simulations, Adv. Protein Chem. 66: 27-85 (2003). In some cases, this solvent-accessible surface area (SASA) may be considered to be accessible by the peptide ligands.

In some embodiments, the binding pocket comprises a plurality of hydrogen bond acceptors configured to form one or more hydrogen bonds in the presence of the amino acid ligand. In some embodiments, the binding pocket forms at least two (e.g., at least three, at least four, at least five, 2-10, 4-10, 5-15, 5-10) hydrogen bonds with an amino acid ligand. Methods for determining hydrogen bond interactions between a binding pocket and a ligand are known in the art and will be apparent to a skilled person in view of the present disclosure. For example, in some embodiments, hydrogen bond interactions are determined by computational modeling as described in the Examples herein (e.g., using atomic coordinates for protein-ligand structural data derived from X-ray crystallography, or through computational modeling to predict the protein-ligand three-dimensional structure).

In some embodiments, the plurality of hydrogen bond acceptors include one or more atoms of a side-chain of an amino acid residue in the binding pocket. For example, in some embodiments, the binding pocket comprises at least three negatively charged amino acid side-chains, each of which (e.g., aspartate, glutamate) forms a hydrogen bond with an amino acid ligand. In some embodiments, at least one of the negatively charged amino acid side-chains forms a hydrogen bond with an amino terminus of the amino acid ligand. In some embodiments, the binding pocket comprises at least one polar uncharged amino acid side-chain that forms a hydrogen bond with an amino acid ligand. In some embodiments, the plurality of hydrogen bond acceptors include one or more atoms of the polypeptide backbone (e.g., backbone carbonyl) in the binding pocket.

In some embodiments, the binding pocket forms one or more hydrogen bonds with a side chain of an amino acid ligand. For example, in some embodiments, the binding pocket forms one or more hydrogen bonds with a side chain of a terminal amino acid of an amino acid ligand (e.g., a polypeptide). In some embodiments, the binding pocket forms one or more hydrogen bonds with the polypeptide backbone of an amino acid ligand (e.g., a polypeptide). In some embodiments, the binding pocket forms one or more hydrogen bonds with a terminal amino acid and one or more amino acids contiguous to the terminal amino acid in a polypeptide (e.g., amino acids at position 1 and at positions 2, 3, 4, and/or 5 relative to the polypeptide terminus).

In some embodiments, the binding pocket comprises a plurality of van der Waals contact positions configured to form van der Waals interactions in the presence of the amino acid ligand. Methods for determining van der Waals interactions between a binding pocket and a ligand are known in the art and will be apparent to a skilled person in view of the present disclosure. For example, in some embodiments, van der Waals interactions are determined by computational modeling as described in the Examples herein (e.g., using atomic coordinates for protein-ligand structural data derived from X-ray crystallography, or through computational modeling to predict the protein-ligand three-dimensional structure).

In some embodiments, the van der Waals contact positions comprise a plurality of atoms (e.g., 2-30, 5-25, 10-20, 2-10, 5-10) configured to form hydrophobic interactions with an amino acid ligand. In some embodiments, one or more atoms of the plurality of atoms are non-polar atoms.

In some embodiments, the amino acid ligand is a polypeptide comprising at least three amino acids. In some embodiments, the amino acid ligand comprises an N-terminal amino acid of a polypeptide. In some embodiments, the N-terminal amino acid is arginine. In some embodiments, the amino acid binding protein is at least 50 amino acids in length, at least 75 amino acids in length, at least 100 amino acids in length, 50-250 amino acids in length, 50-150 amino acids in length, or 100-200 amino acids in length.

In some embodiments, the loop between β2 and α2 comprises three or more negatively charged amino acids. In some embodiments, the loop between β2 and α2 comprises four or more negatively charged amino acids. In some embodiments, at least three negatively charged amino acids of the loop between β2 and α2 form a hydrogen bond with the amino acid ligand. In some embodiments, at least one negatively charged amino acid of the loop between β2 and α2 forms a hydrogen bond with an amino terminus of the amino acid ligand. In some embodiments, the negatively charged amino acids are selected from aspartate and glutamate.

In some embodiments, at least one amino acid of α2 forms a hydrogen bond with the amino acid ligand. In some embodiments, α2 comprises one or more polar uncharged amino acids. In some embodiments, at least one polar uncharged amino acid of α2 forms a hydrogen bond with a side chain of the amino acid ligand.

In some embodiments, the loop between β1 and α1 comprises an amino acid sequence that is at least 80% identical to a sequence of amino acids 27-42 of SEQ ID NO: 2. In some embodiments, the binding pocket is formed by amino acids at one or more positions corresponding to amino acids 31, 32, and 34-36 of SEQ ID NO: 2.

In some embodiments, the loop between α1 and β2 comprises an amino acid sequence that is at least 50% identical to a sequence of amino acids 47-50 of SEQ ID NO: 2. In some embodiments, the amino acid sequence comprises an amino acid substitution at a position corresponding to T47 of SEQ ID NO: 2. In some embodiments, the amino acid substitution is T47 L.

In some embodiments, β2-α2 comprises an amino acid sequence that is at least 80% identical to a sequence of amino acids 51-71 of SEQ ID NO: 2. In some embodiments, the binding pocket is formed by amino acids at one or more positions corresponding to amino acids 63, 65, 68, 70, and 71 of SEQ ID NO: 2. In some embodiments, the amino acid sequence comprises an amino acid substitution at one or more positions corresponding to 163 and E70 of SEQ ID NO: 2. In some embodiments, the amino acid substitution is selected from 163E and E70T.

In some embodiments, the structural equivalent is a structure having a root-mean-square difference of no more than 5.0 Å where at least 80% of secondary structure alpha-carbon atoms are aligned with the structure of Formula (II). In some embodiments, the structural equivalent is a structure having a root-mean-square difference of no more than 4.0 Å, no more than 3.0 Å, no more than 2.0 Å, or no more than 1.0 Å where at least 80% of secondary structure alpha-carbon atoms are aligned with the structure of Formula (II).

Methods for identifying a structural equivalent to the structure of Formula (II) are known in the art and will be apparent to a skilled person in view of the present disclosure. For example, in some embodiments, a structural equivalent to Formula (II) is a structure having a root-mean-square difference of no more than 5.0 Å where at least 80% of secondary structure alpha-carbon atoms are aligned with a three-dimensional protein structure having an amino acid sequence selected from any one of PS1101-1122, PS1218-1221, and PS1351-1398 (SEQ ID NOs: 447-468, 564-567, and 694-741) (e.g., PS1122 (SEQ ID NO: 468), PS1381 (SEQ ID NO: 724)). A protein structure comparison may be performed by determining a three-dimensional structure for a protein having an amino acid sequence selected from any one of PS1101-1122, PS1218-1221, and PS1351-1398 (SEQ ID NOs: 447-468, 564-567, and 694-741) (e.g., PS1122 (SEQ ID NO: 468), PS1381 (SEQ ID NO: 724)), and comparing the three-dimensional structure for the protein to a candidate protein structure to determine whether the candidate protein is a structural equivalent. A three-dimensional protein structure can be determined, for example, using atomic coordinates derived from X-ray crystallography, or through computational modeling to predict the three-dimensional structure of a protein having an amino acid sequence selected from any one of PS1101-1122, PS1218-1221, and PS1351-1398 (SEQ ID NOs: 447-468, 564-567, and 694-741) (e.g., PS1122 (SEQ ID NO: 468), PS1381 (SEQ ID NO: 724)). Methods for comparing protein structures and determining values for root-mean-square difference are known in the art (see, for example, Kufareva I, Abagyan R. Methods of protein structure comparison. Methods Mol Biol. 2012; 857:231-57).

### C. Ntaq1-Homologous Recognizers

In some embodiments, an amino acid recognizer of the disclosure binds an amino acid ligand (e.g., a polypeptide) comprising an N-terminal amino acid selected from glutamine, asparagine, glutamate, aspartate, cysteine-S-acetamide, or a modified variant thereof (e.g., a post-translationally modified variant thereof, an oxidized variant thereof). In some embodiments, the amino acid recognizer comprises an amino acid binding protein derived from an Ntaq1 protein, such as Scleropages formosus Ntaq1 protein. For example, in some embodiments, the amino acid binding protein is an engineered variant comprising one or more modifications relative to SEQ ID NO: 3 as described herein.

In some embodiments, the amino acid binding protein binds N-terminal glutamine with a dissociation constant (K_{D}) of less than 2,000 nM, less than 1,500 nM, less than 1,000 nM, less than 750 nM, less than 500 nM, less than 250 nM, less than 150 nM, less than 100 nM, less than 50 nM, 10-2,000 nM, 25-1,000 nM, 50-500 nM, 10-100 nM, 25-250 nM, or 50-150 nM.

In some embodiments, the amino acid binding protein binds one or more types of N-terminal amino acids (e.g., glutamine, asparagine, glutamate, aspartate, cysteine-S-acetamide, or a modified variant thereof), where each type of binding interaction is characterized by a dissociation rate (k_{off}) of at least 0.1 s⁻¹. In some embodiments, the dissociation rate is between about 0.1 s⁻¹ and about 1,000 s⁻¹ (e.g., between about 0.5 s⁻¹ and about 500 s⁻¹, between about 0.1 s⁻¹ and about 100 s⁻¹, between about 1 s⁻¹ and about 100 s⁻¹, or between about 0.5 s⁻¹ and about 50 s⁻¹). In some embodiments, the dissociation rate is between about 0.5 s⁻¹ and about 20 s⁻¹. In some embodiments, the dissociation rate is between about 2 s⁻¹ and about 20 s⁻¹. In some embodiments, the dissociation rate is between about 0.5 s⁻¹ and about 2 s⁻¹.

In some aspects, the disclosure provides a recombinant or synthetic amino acid binding protein having an amino acid sequence that is at least 40%, at least 50%, at least 60%, at least 70%, at least 80%, at least 85%, at least 90%, at least 95%, at least 98%, or 100% identical to a sequence selected from any one of PS1258-1260, PS1315-1318, PS1457-1478, PS1480-1499, PS1633-1656, PS1737-1758, PS1821-1898, PS2014-2057, and PS2116-2137 (SEQ ID NOs: 604-606, 660-663, 792-833, and 836-1025). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS1258-1260, PS1315-1318, PS1457-1478, PS1480-1499, PS1633-1656, PS1737-1758, PS1821-1898, PS2014-2057, and PS2116-2137 (SEQ ID NOs: 604-606, 660-663, 792-833, and 836-1025). In some embodiments, the amino acid sequence is at least 40%, at least 50%, at least 60%, at least 70%, at least 80%, at least 85%, at least 90%, at least 95%, at least 98%, or 100% identical to PS1259 (SEQ ID NO: 605). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to PS1259 (SEQ ID NO: 605). In some embodiments, the amino acid sequence is at least 40%, at least 50%, at least 60%, at least 70%, at least 80%, at least 85%, at least 90%, at least 95%, at least 98%, or 100% identical to PS2132 (SEQ ID NO: 1020). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to PS2132 (SEQ ID NO: 1020).

In some aspects, the disclosure provides a recombinant or synthetic amino acid binding protein having an amino acid sequence that is at least 80% identical (e.g., at least 85%, at least 90%, at least 95%, at least 98%, 80-98%, 80-95%, 80-90%, 85-95%, or 90-98% identical) to SEQ ID NO: 3, where the amino acid sequence comprises an amino acid substitution at one or more positions corresponding to S22, C23, Y24, C25, E26, S39, W75, D76, Y77, H78, C85, N120, H145, and M146 of SEQ ID NO: 3.

In some embodiments, the amino acid sequence comprises an amino acid substitution at positions corresponding to C25 and H78. In some embodiments, the amino acid sequence comprises an amino acid substitution at positions corresponding to S22, C25, H78 C85, and N120. In some embodiments, the amino acid substitution is selected from S22E, C25S, H78Q, H78K, C85T, N120R, and M146E. In some embodiments, the amino acid substitution is selected from C25S, H78Q, and M146E. In some embodiments, the amino acid substitution is selected from C25S and H78Q. In some embodiments, the amino acid sequence comprises an amino acid substitution at a position corresponding to H78, where the amino acid substitution is H78Q. In some embodiments, the amino acid sequence comprises an amino acid substitution at a position corresponding to H78, where the amino acid substitution is H78K.

In some aspects, the disclosure provides a recombinant or synthetic amino acid binding protein comprising a structure of Formula (III) or a structural equivalent thereof:

α1-α2-α3β1-β2-β3-β4-β5-α4-β6α5-α6 (III),

wherein: each of α1, α2, α3, α4, α5, and α6 is an alpha-helix; each of β1, β2, β3, β4, β5, and β6 is a beta-strand; each instance of “-” is a loop; and at least a portion of each of α2, β3, β4, α5, the loop between α1 and α2, and the loop between β3 and β4 form a binding pocket for an amino acid ligand.

In some embodiments, the binding pocket comprises one or more of the following: (i) a volume of approximately 160 Å³, (ii) an electrostatic potential of −2.0 RTe_{c}⁻¹ or less, (iii) a plurality of hydrogen bond acceptors or donors configured to form one or more hydrogen bonds in the presence of the amino acid ligand, (iv) a plurality of van der Waals contact positions configured to form van der Waals interactions in the presence of the amino acid ligand, and (v) at least one negatively charged amino acid and at least one positively charged amino acid. In some embodiments, the binding pocket comprises one, two, three, or four of (i), (ii), (iii), (iv), and (v). In some embodiments, the binding pocket comprises (i), (ii), (iii), (iv), and (v).

In some embodiments, the binding pocket comprises a volume of approximately 160 Å³. In some embodiments, the volume of the binding pocket is at least 140 Å³, at least 150 Å³, at least 160 Å³, at least 170 Å³, or at least 180 Å³. In some embodiments, the volume of the binding pocket is no more than 180 Å³, no more than 170 Å³, no more than 160 Å³, no more than 150 Å³, or no more than 140 Å³. In some embodiments, the volume of the binding pocket is in a range from 140 Å³ to 160 Å³, 140 Å³ to 170 Å³, 140 Å³ to 180 Å³, 150 Å³ to 170 Å³, 150 Å³ to 180 Å³, 160 Å³ to 180 Å³, or 170 Å³ to 180 Å³. Methods for determining volume of a binding pocket are known in the art and will be apparent to a skilled person in view of the present disclosure. For example, in some embodiments, the volume of the binding pocket is determined using software configured to measure geometric and topological properties of proteins. In some embodiments, the software may scan a protein surface using a specified probe radius to measure the volume of any cavities that directly overlap with a binding site or indirectly overlap with the binding site through adjacent cavities within van der Waals contact of one another. A non-limiting example of a suitable probe radius is a solvent probe radius (e.g., about 1.4 Å). A non-limiting example of suitable software is Computed Atlas of Surface Topography of proteins (CASTp). See, e.g., W. Tian et al., CASTp 3.0: Computed atlas of surface topography of proteins. Nucleic Acids Res. 46, W363-W367 (2018), the relevant content of which is incorporated herein by reference.

In some embodiments, the binding pocket has an electrostatic potential of −2.0 RTe_{c}⁻¹ or less. In some embodiments, the electrostatic potential of the binding pocket is at least −3 RTe_{c}⁻¹, at least −2 RTe_{c}⁻¹, or at least −1 RTe_{c}⁻¹. In some embodiments, the electrostatic potential of the binding pocket is −1 RTe_{c}⁻¹ or less, −2 RTe_{c}⁻¹ or less, or −3 RTe_{c}⁻¹ or less. In some embodiments, the electrostatic potential of the binding pocket is in a range from −1 RTe_{c}⁻¹ to −2 RTe_{c}⁻¹, −1 RTe_{c}⁻¹ to −3 RTe_{c}⁻¹, or −2 RTe_{c}⁻¹ to −3 RTe_{c}⁻¹. Methods for determining electrostatic potential of a binding pocket are known in the art and will be apparent to a skilled person in view of the present disclosure. For example, in some embodiments, the Adaptive Poisson-Boltzmann Solver (APBS) tool in PyMOL (PyMOL Molecular Graphics System, Version 2.0 Schrödinger, LLC) may be used (e.g., with default parameters) to calculate the electrostatic surface potential of a binding pocket using pdb2pgr with the AMBER forcefield to assign protonation states. See, e.g., T. J. Dolinsky et al., PDB2PQR: An automated pipeline for the setup of Poisson-Boltzmann electrostatics calculations, Nucleic Acids Res., 32:W665-7 (2004); M. G. Lerner et al., APBS plugin for PyMOL—Version 2.4 (University of Michigan, Ann Arbor, M I, 2006); J. W. Ponder et al., Force fields for protein simulations, Adv. Protein Chem. 66: 27-85 (2003). In some cases, this solvent-accessible surface area (SASA) may be considered to be accessible by the peptide ligands.

In some embodiments, the binding pocket comprises a plurality of hydrogen bond acceptors or donors configured to form one or more hydrogen bonds in the presence of the amino acid ligand. In some embodiments, the binding pocket forms at least two (e.g., at least three, at least four, at least five, 2-10, 4-10, 5-15, 5-10) hydrogen bonds with an amino acid ligand. Methods for determining hydrogen bond interactions between a binding pocket and a ligand are known in the art and will be apparent to a skilled person in view of the present disclosure. For example, in some embodiments, hydrogen bond interactions are determined by computational modeling as described in the Examples herein (e.g., using atomic coordinates for protein-ligand structural data derived from X-ray crystallography, or through computational modeling to predict the protein-ligand three-dimensional structure).

In some embodiments, the plurality of hydrogen bond acceptors or donors include one or more atoms of a side-chain of an amino acid residue in the binding pocket. For example, in some embodiments, the binding pocket comprises at least three negatively charged amino acid side-chains, each of which (e.g., aspartate, glutamate) forms a hydrogen bond with an amino acid ligand. In some embodiments, at least one of the negatively charged amino acid side-chains forms a hydrogen bond with an amino terminus of the amino acid ligand. In some embodiments, the binding pocket comprises at least one polar uncharged amino acid side-chain (e.g., serine, glutamine) that forms a hydrogen bond with an amino acid ligand. In some embodiments, the plurality of hydrogen bond acceptors or donors include one or more atoms of the polypeptide backbone (e.g., backbone carbonyl) in the binding pocket. In some embodiments, the binding pocket comprises least one negatively charged amino acid side-chain and at least one positively charged amino acid side-chain, each of which forms a hydrogen bond with an amino acid ligand. In some embodiments, the at least one negatively charged amino acid side-chain (e.g., aspartate, glutamate) forms a hydrogen bond with a backbone atom (e.g., nitrogen) of the amino acid ligand. In some embodiments, the at least one positively charged amino acid side-chain (e.g., lysine) forms a hydrogen bond with a side chain atom of the amino acid ligand.

In some embodiments, the binding pocket forms one or more hydrogen bonds with a side chain of an amino acid ligand. For example, in some embodiments, the binding pocket forms one or more hydrogen bonds with a side chain of a terminal amino acid of an amino acid ligand (e.g., a polypeptide). In some embodiments, the binding pocket forms one or more hydrogen bonds with the polypeptide backbone of an amino acid ligand (e.g., a polypeptide). In some embodiments, the binding pocket forms one or more hydrogen bonds with a terminal amino acid and one or more amino acids contiguous to the terminal amino acid in a polypeptide (e.g., amino acids at position 1 and at positions 2, 3, 4, and/or 5 relative to the polypeptide terminus).

In some embodiments, the binding pocket comprises a plurality of van der Waals contact positions configured to form van der Waals interactions in the presence of the amino acid ligand. Methods for determining van der Waals interactions between a binding pocket and a ligand are known in the art and will be apparent to a skilled person in view of the present disclosure. For example, in some embodiments, van der Waals interactions are determined by computational modeling as described in the Examples herein (e.g., using atomic coordinates for protein-ligand structural data derived from X-ray crystallography, or through computational modeling to predict the protein-ligand three-dimensional structure).

In some embodiments, the van der Waals contact positions comprise a plurality of atoms (e.g., 2-30, 5-25, 10-20, 2-10, 5-10) configured to form hydrophobic interactions with an amino acid ligand. In some embodiments, one or more atoms of the plurality of atoms are non-polar atoms.

In some embodiments, the amino acid ligand is a polypeptide comprising at least three amino acids. In some embodiments, the amino acid ligand comprises an N-terminal amino acid of a polypeptide. In some embodiments, the N-terminal amino acid is selected from glutamine, asparagine, glutamate, aspartate, and cysteine-S-acetamide. In some embodiments, the amino acid ligand is a polypeptide comprising an N-terminal glutamine or asparagine. In some embodiments, the binding pocket comprises (i), (ii), (iii), and (iv), and the amino acid ligand is a polypeptide comprising an N-terminal glutamine or asparagine. In some embodiments, the amino acid ligand is a polypeptide comprising an N-terminal glutamate. In some embodiments, the binding pocket comprises (i), (ii), (iii), (iv), and (v), and the amino acid ligand is a polypeptide comprising an N-terminal glutamate. In some embodiments, the amino acid binding protein is at least 50 amino acids in length, at least 75 amino acids in length, at least 100 amino acids in length, 50-250 amino acids in length, 50-150 amino acids in length, or 100-200 amino acids in length.

In some embodiments, each of α2 and β4 comprises at least one polar uncharged amino acid that forms a hydrogen bond with the amino acid ligand. In some embodiments, the at least one polar uncharged amino acid of α2 is serine. In some embodiments, the at least one polar uncharged amino acid of β4 is glutamine.

In some embodiments, α2 and the loop between α1 and α2 comprises an amino acid sequence that is at least 80% identical to a sequence of amino acids 18-40 of SEQ ID NO: 3. In some embodiments, the binding pocket is formed by amino acids at one or more positions corresponding to amino acids 23-26 of SEQ ID NO: 3. In some embodiments, the amino acid sequence comprises an amino acid substitution at a position corresponding to C25 of SEQ ID NO: 3. In some embodiments, the amino acid substitution is C25S.

In some embodiments, β3-β4 comprises an amino acid sequence that is at least 80% identical to a sequence of amino acids 73-85 of SEQ ID NO: 3. In some embodiments, the binding pocket is formed by amino acids at one or more positions corresponding to amino acids 75-78 of SEQ ID NO: 3. In some embodiments, the amino acid sequence comprises an amino acid substitution at a position corresponding to H78 of SEQ ID NO: 3. In some embodiments, the amino acid substitution is H78Q.

In some embodiments, α6 comprises an amino acid sequence that is at least 66% identical to a sequence of amino acids 144-146 of SEQ ID NO: 3. In some embodiments, the binding pocket is formed by amino acids at one or more positions corresponding to amino acids 145-146 of SEQ ID NO: 3.

In some embodiments, the amino acid binding protein comprises a structure of Formula (III-A) or a structural equivalent thereof:

α1-α2-α3β1-β2-β3-β4-β5-α4-β6α5-α6-α7-β7α8 (III-A),

wherein: each of α7 and α8 is an alpha-helix; and β7 is a beta-strand.

In some embodiments, the amino acid binding protein comprises a structure of Formula (III-B) or a structural equivalent thereof:

α1-α2-α3β1-β2-β3-β4 (III-B),

wherein: each of α1, α2, and α3 is an alpha-helix; each of β1, β2, β3, and β4 is a beta-strand; each instance of “-” is a loop; and at least a portion of each of α2, β3, β4, the loop between α1 and α2, and the loop between β3 and β4 form a binding pocket for an amino acid ligand.

In some embodiments, the binding pocket comprises: (i) at least one negatively charged amino acid configured to form a hydrogen bond with the amino acid ligand, and (ii) at least one positively charged amino acid configured to form a hydrogen bond with the amino acid ligand. In some embodiments, the amino acid ligand is a polypeptide comprising at least three amino acids. In some embodiments, the amino acid ligand comprises an N-terminal amino acid of a polypeptide. In some embodiments, the N-terminal amino acid is glutamate.

In some embodiments, the at least one negatively charged amino acid forms the hydrogen bond with a backbone atom of the amino acid ligand. In some embodiments, the at least one positively charged amino acid forms the hydrogen bond with a side chain atom of the amino acid ligand. In some embodiments, the at least one negatively charged amino acid forms the hydrogen bond with a backbone atom of the amino acid ligand, and the at least one positively charged amino acid forms the hydrogen bond with a side chain atom of the amino acid ligand. In some embodiments, a side chain atom of the at least one negatively charged amino acid forms the hydrogen bond with the backbone atom of the amino acid ligand. In some embodiments, a side chain atom of the at least one positively charged amino acid forms the hydrogen bond with a side chain atom of the amino acid ligand. In some embodiments, a side chain atom of the at least one negatively charged amino acid forms the hydrogen bond with the backbone atom of the amino acid ligand, and a side chain atom of the at least one positively charged amino acid forms the hydrogen bond with a side chain atom of the amino acid ligand.

In some embodiments, α2 comprises the at least one negatively charged amino acid. In some embodiments, β4 comprises the at least one positively charged amino acid. In some embodiments, α2 comprises the at least one negatively charged amino acid, and β4 comprises the at least one positively charged amino acid. In some embodiments, the at least one negatively charged amino acid comprises glutamate. In some embodiments, the at least one positively charged amino acid comprises lysine. In some embodiments, the at least one negatively charged amino acid comprises glutamate, and the at least one positively charged amino acid comprises lysine. In some embodiments, the at least one negatively charged amino acid corresponds to E26 of SEQ ID NO: 3. In some embodiments, the at least one positively charged amino acid is a lysine substitution at a position corresponding to H78 of SEQ ID NO: 3. In some embodiments, the at least one negatively charged amino acid corresponds to E26 of SEQ ID NO: 3, and the at least one positively charged amino acid is a lysine substitution at a position corresponding to H78 of SEQ ID NO: 3.

In some embodiments, α1-α2 comprises an amino acid sequence that is at least 80% identical to a sequence of amino acids 15-39 of SEQ ID NO: 3. In some embodiments, the amino acid sequence comprises amino acid substitutions at positions corresponding to S22 and C25 of SEQ ID NO: 3. In some embodiments, the amino acid substitutions are S22E and C25S. In some embodiments, β3-β4 comprises an amino acid sequence that is at least 80% identical to a sequence of amino acids 73-85 of SEQ ID NO: 3. In some embodiments, the amino acid sequence comprises amino acid substitutions at positions corresponding to H78 and C85 of SEQ ID NO: 3. In some embodiments, the amino acid substitutions are H78K and C85T.

In some embodiments, the structural equivalent is a structure having a root-mean-square difference of no more than 5.0 Å where at least 80% of secondary structure alpha-carbon atoms are aligned with the structure of Formula (III), (III-A), or (III-B). In some embodiments, the structural equivalent is a structure having a root-mean-square difference of no more than 4.0 Å, no more than 3.0 Å, no more than 2.0 Å, or no more than 1.0 Å where at least 80% of secondary structure alpha-carbon atoms are aligned with the structure of Formula (III), (III-A), or (III-B).

Methods for identifying a structural equivalent to the structure of Formula (III), (III-A), or (III-B) are known in the art and will be apparent to a skilled person in view of the present disclosure. For example, in some embodiments, a structural equivalent to Formula (III), (III-A), or (III-B) is a structure having a root-mean-square difference of no more than 5.0 Å where at least 80% of secondary structure alpha-carbon atoms are aligned with a three-dimensional protein structure having an amino acid sequence selected from any one of PS1258-1260, PS1315-1318, PS1457-1478, PS1480-1499, PS1633-1656, PS1737-1758, PS1821-1898, PS2014-2057, and PS2116-2137 (SEQ ID NOs: 604-606, 660-663, 792-833, and 836-1025). A protein structure comparison may be performed by determining a three-dimensional structure for a protein having an amino acid sequence selected from any one of PS1258-1260, PS1315-1318, PS1457-1478, PS1480-1499, PS1633-1656, PS1737-1758, PS1821-1898, PS2014-2057, and PS2116-2137 (SEQ ID NOs: 604-606, 660-663, 792-833, and 836-1025), and comparing the three-dimensional structure for the protein to a candidate protein structure to determine whether the candidate protein is a structural equivalent. A three-dimensional protein structure can be determined, for example, using atomic coordinates derived from X-ray crystallography, or through computational modeling to predict the three-dimensional structure of a protein having an amino acid sequence selected from any one of PS1258-1260, PS1315-1318, PS1457-1478, PS1480-1499, PS1633-1656, PS1737-1758, PS1821-1898, PS2014-2057, and PS2116-2137 (SEQ ID NOs: 604-606, 660-663, 792-833, and 836-1025). Methods for comparing protein structures and determining values for root-mean-square difference are known in the art (see, for example, Kufareva I, Abagyan R. Methods of protein structure comparison. Methods Mol Biol. 2012; 857:231-57).

### D. BIR Domain-Homologous Recognizers

In some embodiments, an amino acid recognizer of the disclosure binds an amino acid ligand (e.g., a polypeptide) comprising an N-terminal alanine. In some embodiments, the amino acid recognizer comprises an amino acid binding protein derived from a Baculoviral IAP repeat-containing (BIR) protein, such as Homo sapiens BIR3 domain protein. For example, in some embodiments, the amino acid binding protein is an engineered variant comprising one or more modifications relative to SEQ ID NO: 511 as described herein.

In some embodiments, the amino acid binding protein binds N-terminal alanine with a dissociation constant (K_{D}) of less than 2,000 nM, less than 1,500 nM, less than 1,000 nM, less than 750 nM, less than 500 nM, less than 250 nM, less than 150 nM, less than 100 nM, less than 80 nM, 10-2,000 nM, 25-1,000 nM, 50-500 nM, 10-150 nM, 25-75 nM, or 50-60 nM.

In some embodiments, the amino acid binding protein binds N-terminal alanine, where the binding interaction is characterized by a dissociation rate (k_{off}) of at least 0.1 s⁻¹. In some embodiments, the dissociation rate is between about 0.1 s⁻¹ and about 1,000 s⁻¹ (e.g., between about 0.5 s⁻¹ and about 500 s⁻¹, between about 0.1 s⁻¹ and about 100 s⁻¹, between about 1 s⁻¹ and about 100 s⁻¹, or between about 0.5 s⁻¹ and about 50 s⁻¹). In some embodiments, the dissociation rate is between about 0.5 s⁻¹ and about 20 s⁻¹. In some embodiments, the dissociation rate is between about 2 s⁻¹ and about 20 s⁻¹. In some embodiments, the dissociation rate is between about 0.5 s⁻¹ and about 2 s⁻¹

In some aspects, the disclosure provides a recombinant or synthetic amino acid binding protein having an amino acid sequence that is at least 40%, at least 50%, at least 60%, at least 70%, at least 80%, at least 85%, at least 90%, at least 95%, at least 98%, or 100% identical to a sequence selected from any one of PS1165-1166 (SEQ ID NOs: 511-512), PS1267 (SEQ ID NO: 613), and PS1399-1424 (SEQ ID NOs: 742-767). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS1165-1166 (SEQ ID NOs: 511-512), PS1267 (SEQ ID NO: 613), and PS1399-1424 (SEQ ID NOs: 742-767). In some embodiments, the amino acid sequence is at least 40%, at least 50%, at least 60%, at least 70%, at least 80%, at least 85%, at least 90%, at least 95%, at least 98%, or 100% identical to PS1165 (SEQ ID NO: 511). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to PS1165 (SEQ ID NO: 511).

In some aspects, the disclosure provides a recombinant or synthetic amino acid binding protein having an amino acid sequence that is at least 80% identical to PS1165 (SEQ ID NO: 511) and comprising one or more labels as described herein. In some embodiments, the amino acid sequence is at least 85%, at least 90%, at least 95%, at least 98%, or 100% identical to PS1165 (SEQ ID NO: 511). In some embodiments, the amino acid sequence is between about 80% and about 100% (e.g., 80-98%, 80-95%, 80-90%, 85-95%, 90-98%, 90-100%, or 95-100%) identical to PS1165 (SEQ ID NO: 511).

### E. Tandem Recognizers

In some embodiments, an amino acid recognizer comprises a single polypeptide having tandem copies of two or more amino acid binding proteins, where at least one of the two or more amino acid binding proteins is an amino acid binding protein of the disclosure. As used herein, in some embodiments, a tandem arrangement or orientation of elements in a molecule refers to an end-to-end joining of each element to the next element in a linear fashion such that the elements are fused in series. For example, in some embodiments, a polypeptide having tandem copies of two amino acid binding proteins refers to a fusion polypeptide in which the C-terminus of one protein is fused to the N-terminus of the other protein. Similarly, a polypeptide having tandem copies of two or more amino acid binding proteins refers to a fusion polypeptide in which the C-terminus of a first protein is fused to the N-terminus of a second protein, the C-terminus of the second protein is fused to the N-terminus of a third protein, and so forth. Such fusion polypeptides can comprise multiple copies of the same amino acid binding protein or multiple copies of different amino acid binding proteins. In some embodiments, a fusion polypeptide of the application has at least two and up to ten amino acid binding proteins (e.g., at least 2 binders and up to eight, six, five, four, or three binders). In some embodiments, a fusion polypeptide of the application has five or fewer amino acid binding proteins (e.g., two, three, four, or five amino acid binding proteins).

In some embodiments, a fusion polypeptide is provided by expression of a single coding sequence containing segments encoding monomeric amino acid binding protein subunits separated by segments encoding flexible linkers, where expression of the single coding sequence produces a single full-length polypeptide having two or more independent binding sites. In some embodiments, one or more of the monomeric subunits are ClpS-homologous proteins, UBR-homologous proteins, or Ntaq1-homologous proteins. In some embodiments, the monomeric subunits may be identical or non-identical. Where non-identical, the monomeric subunits may be distinct variants of the same parent-homologous protein, or they may be derived from different parent-homologous proteins. In some embodiments, a fusion polypeptide comprises two or more ClpS-homologous monomers, two or more UBR-homologous monomers, or two or more Ntaq1-homologous monomers.

In some embodiments, at least one amino acid binding protein of a fusion polypeptide has an amino acid sequence selected from Table 1 (or having an amino acid sequence that has at least 50%, at least 60%, at least 70%, at least 80%, 80-90%, 90-95%, 95-99%, or higher, amino acid sequence identity to an amino acid sequence selected from Table 1). In some embodiments, each amino acid binding protein of a fusion polypeptide has an amino acid sequence that is at least 80% (e.g., 80-90%, 90-95%, 95-99%, or higher) identical to an amino acid sequence selected from Table 1 (or having an amino acid sequence that has at least 50%, at least 60%, at least 70%, at least 80%, 80-90%, 90-95%, 95-99%, or higher, amino acid sequence identity to an amino acid sequence selected from Table 1). In some embodiments, an amino acid binding protein of a fusion polypeptide is modified and includes one or more amino acid deletions, additions, or mutations relative to a sequence set forth in Table 1. In some embodiments, an amino acid binding protein of a fusion polypeptide includes a deletion, addition, or mutation of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acids (which may or may not be consecutive amino acids) relative to a sequence set forth in Table 1.

In some embodiments, amino acid binding proteins of a fusion polypeptide recognize the same set of one or more amino acids. In some embodiments, amino acid binding proteins of a fusion polypeptide recognize a distinct set of one or more amino acids. In some embodiments, amino acid binding proteins of a fusion polypeptide recognize an overlapping set of amino acids. In some embodiments, where the amino acid binding proteins of a fusion polypeptide recognize the same amino acid, they may recognize the amino acid with the same characteristic pulsing pattern or with different characteristic pulsing patterns.

In some embodiments, amino acid binding proteins of a fusion polypeptide are joined end-to-end, either by a covalent bond or a linker that covalently joins the C-terminus of one protein to the N-terminus of another protein. In the context of fusion polypeptides of the application, a linker refers to one or more amino acids within a fusion polypeptide that joins two amino acid binding proteins and that does not form part of the polypeptide sequence corresponding to either of the two proteins. In some embodiments, a linker comprises at least two amino acids (e.g., at least 2, 3, 4, 5, 6, 8, 10, 15, 25, 50, 100, or more, amino acids). In some embodiments, a linker comprises up to 5, up to 10, up to 15, up to 25, up to 50, or up to 100, amino acids. In some embodiments a linker comprises between about 2 and about 200 amino acids (e.g., between about 2 and about 100, between about 5 and about 50, between about 2 and about 20, between about 5 and about 20, or between about 2 and about 30, amino acids).

Accordingly, in some aspects, the disclosure provides an amino acid recognizer comprising a polypeptide having a first amino acid binding protein and a second amino acid binding protein joined end-to-end, where the first and second amino acid binding proteins are separated by a linker comprising at least two amino acids.

In some embodiments, each of the first and second amino acid binding proteins independently has an amino acid sequence that is at least 80% identical (e.g., at least 85%, at least 90%, at least 95%, at least 98%, 80-100%, 85-100%, 90-100%, 95-100%, or 100% identical) to PS961 (SEQ ID NO: 314). In some embodiments, the amino acid recognizer comprises a polypeptide having an amino acid sequence that is at least 80%, at least 85%, at least 90%, at least 95%, at least 98%, 80-100%, 85-100%, 90-100%, 95-100%, or 100% identical to a sequence selected from any one of PS1038, PS1222, and PS1223 (SEQ ID NOs: 389, 568, and 569).

In some embodiments, each of the first and second amino acid binding proteins independently has an amino acid sequence that is at least 80% identical (e.g., at least 85%, at least 90%, at least 95%, at least 98%, 80-100%, 85-100%, 90-100%, 95-100%, or 100% identical) to PS1122 (SEQ ID NO: 468). In some embodiments, the amino acid recognizer comprises a polypeptide having an amino acid sequence that is at least 80%, at least 85%, at least 90%, at least 95%, at least 98%, 80-100%, 85-100%, 90-100%, 95-100%, or 100% identical to a sequence selected from any one of PS1219-PS1221 (SEQ ID NOs: 565-567).

In some embodiments, each of the first and second amino acid binding proteins independently has an amino acid sequence that is at least 80% identical (e.g., at least 85%, at least 90%, at least 95%, at least 98%, 80-100%, 85-100%, 90-100%, 95-100%, or 100% identical) to PS1259 (SEQ ID NO: 605). In some embodiments, the amino acid recognizer comprises a polypeptide having an amino acid sequence that is at least 80%, at least 85%, at least 90%, at least 95%, at least 98%, 80-100%, 85-100%, 90-100%, 95-100%, or 100% identical to PS1599 (SEQ ID NO: 835).

In some aspects, the application provides a nucleic acid encoding a single polypeptide having tandem copies of two or more amino acid binding proteins. In some embodiments, the nucleic acid is an expression construct encoding a fusion polypeptide of the application. In some embodiments, an expression construct encodes a fusion polypeptide having at least two and up to ten amino acid binding proteins (e.g., at least two and up to three, four, five, six, seven, eight, nine, or ten amino acid binding proteins). In some embodiments, an expression construct encodes a fusion polypeptide having five or fewer amino acid binding proteins (e.g., two, three, four, or five amino acid binding proteins).

### F. Shielded Recognizers

In accordance with embodiments described herein, single-molecule polypeptide sequencing methods can be carried out by illuminating a surface-immobilized polypeptide with excitation light, and detecting luminescence produced by a label attached to an amino acid recognizer. In some cases, radiative and/or non-radiative decay produced by the label can result in photodamage to the polypeptide, and the inventors have found that photodamage can be mitigated and recognition times extended by incorporation of a shielding element into an amino acid recognizer. See, for example, PCT International Publication No. WO2020102741A1, filed Nov. 15, 2019, and PCT International Publication No. WO2021236983A2, filed May 20, 2021, which describe shielded recognition molecules in detail, the relevant content of which is incorporated by reference in its entirety.

Accordingly, in some aspects, the disclosure provides shielded recognizers comprising at least one amino acid recognizer (e.g., amino acid binding protein) described herein, at least one detectable label, and a shielding element (e.g., a “shield”) that forms a covalent or non-covalent linkage group between the recognizer and label. In some embodiments, a shield forms a covalent or non-covalent linkage group between one or more amino acid binding proteins and one or more labels.

In some embodiments, a shielded recognizer comprises a fusion polypeptide having an amino acid binding protein of the disclosure and a protein shield joined end-to-end (e.g., in a C-terminal to N-terminal fashion). In some embodiments, the protein shield comprises a labeled protein, such as a fluorescent protein or a non-fluorescent protein that comprises a luminescent label.

In some embodiments, the amino acid binding protein and the protein shield are joined end-to-end, either by a covalent bond or a linker that covalently joins the C-terminus of one protein to the N-terminus of the other protein. In some embodiments, a linker in the context of a fusion polypeptide refers to one or more amino acids within the fusion polypeptide that joins the amino acid binding protein and the protein shield and that does not form part of the polypeptide sequence corresponding to either the amino acid binding protein or the protein shield. In some embodiments, a linker comprises at least two amino acids (e.g., at least 2, 3, 4, 5, 6, 8, 10, 15, 25, 50, 100, or more, amino acids). In some embodiments, a linker comprises up to 5, up to 10, up to 15, up to 25, up to 50, or up to 100, amino acids. In some embodiments a linker comprises between about 2 and about 200 amino acids (e.g., between about 2 and about 100, between about 5 and about 50, between about 2 and about 20, between about 5 and about 20, or between about 2 and about 30, amino acids).

In some embodiments, a protein shield of a fusion polypeptide is a protein having a molecular weight of at least 10 kDa. For example, in some embodiments, a protein shield is a protein having a molecular weight of at least 10 kDa and up to 500 kDa (e.g., between about 10 kDa and about 250 kDa, between about 10 kDa and about 150 kDa, between about 10 kDa and about 100 kDa, between about 20 kDa and about 80 kDa, between about 15 kDa and about 100 kDa, or between about 15 kDa and about 50 kDa). In some embodiments, a protein shield of a fusion polypeptide is a protein comprising at least 25 amino acids. For example, in some embodiments, a protein shield is a protein comprising at least 25 and up to 1,000 amino acids (e.g., between about 100 and about 1,000 amino acids, between about 100 and about 750 amino acids, between about 500 and about 1,000 amino acids, between about 250 and about 750 amino acids, between about 50 and about 500 amino acids, between about 100 and about 400 amino acids, or between about 50 and about 250 amino acids).

In some embodiments, a protein shield is a polypeptide comprising one or more tag proteins. In some embodiments, a protein shield is a polypeptide comprising at least two tag proteins. In some embodiments, the at least two tag proteins are the same (e.g., the polypeptide comprises at least two copies of a tag protein sequence). In some embodiments, the at least two tag proteins are different (e.g., the polypeptide comprises at least two different tag protein sequences). Examples of tag proteins include, without limitation, Fasciola hepatica 8-kDa antigen (Fh8), Maltose-binding protein (MBP), N-utilization substance (NusA), Thioredoxin (Trx), Small ubiquitin-like modifier (SUMO), Glutathione-S-transferase (GST), Solubility-enhancer peptide sequences (SET), IgG domain B1 of Protein G (GB1), IgG repeat domain ZZ of Protein A (ZZ), Mutated dehalogenase (HaloTag), Solubility eNhancing Ubiquitous Tag (SNUT), Seventeen kilodalton protein (Skp), Phage T7 protein kinase (T7PK), E. coli secreted protein A (EspA), Monomeric bacteriophage T7 0.3 protein (Orc protein; Mocr), E. coli trypsin inhibitor (Ecotin), Calcium-binding protein (CaBP), Stress-responsive arsenate reductase (ArsC), N-terminal fragment of translation initiation factor IF2 (IF2-domain I), Stress-responsive proteins (e.g., RpoA, SlyD, Tsf, RpoS, PotD, Crr), and E. coli acidic proteins (e.g., msyB, yjgD, rpoD). See, e.g., Costa, S., et al. “Fusion tags for protein solubility, purification and immunogenicity in Escherichia coli: the novel Fh8 system.” Front Microbiol. 2014 Feb. 19; 5:63, the relevant content of which is incorporated herein by reference.

A shielding element of the disclosure can advantageously absorb, deflect, or otherwise block radiative and/or non-radiative decay emitted by a label of an amino acid recognizer. Thus, it should be appreciated that a suitable protein shield of a fusion polypeptide can be readily selected by those skilled in the art. For example, the inventors have demonstrated the use of a variety of types of protein shields in the context of a fusion polypeptide, including polypeptides having an amino acid binding protein fused to an enzyme (e.g., DNA polymerase, glutathione S-transferase), a transport protein (e.g., maltose-binding protein), a fluorescent protein (e.g., GFP), and a commercially available tag protein (e.g., SNAP-Tag®). The inventors have further demonstrated the use of fusion polypeptides having multiple copies of a protein shield oriented in tandem. See, for example, PCT International Publication No. WO2021236983A2, filed May 20, 2021.

Accordingly, in some embodiments, the disclosure provides a fusion polypeptide having one or more tandemly-oriented amino acid binding proteins fused to one or more tandemly-oriented protein shields. In some embodiments, where a fusion polypeptide comprises two or more tandemly-oriented binders and/or two or more tandemly-oriented shields, a terminal end of one of the two or more binders is joined end-to-end with a terminal end of one of the two or more shields. Fusion polypeptides having tandem copies of two or more binders are described elsewhere herein, and in some embodiments, such fusions can further comprise a protein shield joined end-to-end with one of the two or more binders.

Additional example configurations of shielded recognizers and shielding elements (e.g., oligonucleotide shields, avidin protein shields) have been described and are contemplated for use in accordance with the present disclosure. See, for example, PCT International Publication No. WO2020102741A1, filed Nov. 15, 2019, and PCT International Publication No. WO2021236983A2, filed May 20, 2021, the relevant contents of each of which are incorporated herein.

### G. Labels

In some embodiments, an amino acid recognizer of the disclosure comprises one or more labels. In some embodiments, the one or more labels comprise a detectable label, such as a luminescent label or a conductivity label. As described herein, in some embodiments, one or more chemical characteristics of a polypeptide can be determined by monitoring a signal for changes in the signal (e.g., signal pulses) corresponding to binding events between one or more amino acid recognizers and the polypeptide. In some embodiments, an amino acid recognizer comprises a detectable label that produces a change in the signal during a binding event between the amino acid recognizer and the polypeptide. Accordingly, as used herein, a detectable label of an amino acid recognizer can refer to any label capable of producing a detectable change in signal during a binding event between the amino acid recognizer and a polypeptide.

In some embodiments, the one or more labels of an amino acid recognizer comprise a luminescent label. In some embodiments, a luminescent label comprises at least one fluorophore dye molecule (e.g., at least 2, at least 3, at least 4, at least 5, 20 or fewer, 15 or fewer, 10 or fewer fluorophore dye molecules). In some embodiments, a luminescent label comprises at least one FRET pair comprising a donor label and an accepter label. Examples of luminescent labels and their use in accordance with the disclosure are described in detail elsewhere herein.

In some embodiments, the one or more labels of an amino acid recognizer comprise a conductivity label. In some embodiments, the conductivity label is a charge label, such as a charged polymer. Examples of charge labels include dendrimers, nanoparticles, nucleic acids and other polymers having multiple charged groups. In some embodiments, a conductivity label is uniquely identifiable by its net charge (e.g., a net positive charge or a net negative charge), by its charge density, and/or by its number of charged groups.

In some embodiments, the one or more labels of an amino acid recognizer comprise a tag sequence. For example, in some embodiments, an amino acid recognizer comprises a tag sequence that provides one or more functions other than amino acid binding. In some embodiments, a tag sequence comprises at least one biotin ligase recognition sequence that permits biotinylation of the recognizer (e.g., incorporation of one or more biotin molecules, including biotin and bis-biotin moieties). In some embodiments, a tag sequence comprises two biotin ligase recognition sequences oriented in tandem. In some embodiments, a biotin ligase recognition sequence refers to an amino acid sequence that is recognized by a biotin ligase, which catalyzes a covalent linkage between the sequence and a biotin molecule. Each biotin ligase recognition sequence of a tag sequence can be covalently linked to a biotin moiety, such that a tag sequence having multiple biotin ligase recognition sequences can be covalently linked to multiple biotin molecules. A region of a tag sequence having one or more biotin ligase recognition sequences can be generally referred to as a biotinylation tag or a biotinylation sequence. In some embodiments, a bis-biotin or bis-biotin moiety can refer to two biotins bound to two biotin ligase recognition sequences oriented in tandem.

Additional examples of functional sequences in a tag sequence include purification tags, cleavage sites, and other moieties useful for purification and/or modification of recognizers. Table 2 provides a list of non-limiting sequences of tag sequences, any one or more of which may be used in combination with any one of the amino acid recognizers of the application (e.g., in combination with a sequence set forth in Table 1). It should be appreciated that the tag sequences shown in Table 2 are meant to be non-limiting, and recognizers in accordance with the application can include any one or more of the tag sequences (e.g., His-tags and/or biotinylation tags) at the N- or C-terminus of a recognizer polypeptide or at an internal position, split between the N- and C-terminus, or otherwise rearranged as practiced in the art.

In some embodiments, the one or more labels of an amino acid recognizer comprise a biotin moiety. In some embodiments, the biotin moiety comprises at least one biotin molecule (e.g., 1, 2, 3, 4, or more biotin molecules). In some embodiments, the biotin moiety is a bis-biotin moiety. In some embodiments, the biotin moiety comprises at least one biotin molecule attached to at least one biotin ligase recognition sequence. For example, in some embodiments, the one or more labels comprise a tag sequence comprising two biotin ligase recognition sequences oriented in tandem, each biotin ligase recognition sequence having a biotin molecule attached thereto. In some embodiments, the biotin moiety comprises at least one biotin molecule attached to the amino acid recognizer through means other than a tag sequence. For example, in some embodiments, the at least one biotin molecule is chemically conjugated to an amino acid (e.g., an unnatural amino acid) of an amino acid binding protein.

In some embodiments, the one or more labels of an amino acid recognizer comprise one or more polyol moieties (e.g., one or more moieties selected from dextran, polyvinylpyrrolidone, polyethylene glycol, polypropylene glycol, polyoxyethylene glycol, and polyvinyl alcohol). For example, in some embodiments, an amino acid recognizer is PEGylated. In some embodiments, polyol modification (e.g., PEGylation) can limit the extent of non-specific sticking to a substrate (e.g., sequencing chip) surface. In some embodiments, polyol modification can limit the extent of aggregation or interaction between an amino acid recognizer with other recognizers, with a cleaving reagent, or with other species present in a sequencing reaction mixture. PEGylation can be performed by incubating a recognizer (e.g., an amino acid binding protein, such as a ClpS protein) with mPEG4-NHS ester, which labels primary amines such as surface-exposed lysine side chains. Other types of PEG and other methods of polyol modification are known in the art.

It should be appreciated that, in some embodiments, an amino acid recognizer of the disclosure can comprise one or more different types of labels described herein. For example, in some embodiments, an amino acid recognizer comprises one or more labels selected from a detectable label (e.g., a luminescent label, a conductivity label), a tag sequence (e.g., a purification tag, a cleavage site, a biotinylation sequence), a biotin moiety, and a polyol moiety. In some embodiments, an amino acid recognizer comprises a detectable label (e.g., a luminescent label, a conductivity label) and one or more labels selected from a tag sequence (e.g., a purification tag, a cleavage site, a biotinylation sequence), a biotin moiety, and a polyol moiety.

In some embodiments, the one or more labels of an amino acid recognizer comprise a luminescent label. As used herein, a luminescent label is a molecule that absorbs one or more photons and may subsequently emit one or more photons after one or more time durations. In some embodiments, the term is used interchangeably with “label,” “detectable label,” or “luminescent molecule” depending on context. A luminescent label in accordance with certain embodiments described herein may refer to a luminescent label of an amino acid recognizer, a luminescent label of a cleaving reagent (e.g., a peptidase, such as an aminopeptidase), or a luminescent label of another labeled composition described herein.

In some embodiments, a luminescent label comprises a first chromophore and a second chromophore. In some embodiments, an excited state of the first chromophore is capable of relaxation via an energy transfer to the second chromophore. In some embodiments, the energy transfer is a Förster resonance energy transfer (FRET). Such a FRET pair may be useful for providing a luminescent label with properties that make the label easier to differentiate from amongst a plurality of luminescent labels in a mixture, or for providing a binding-induced fluorescence that limits background fluorescence as described elsewhere herein. In yet other embodiments, a FRET pair comprises a first chromophore of a first luminescent label and a second chromophore of a second luminescent label. In certain embodiments, the FRET pair may absorb excitation energy in a first spectral range and emit luminescence in a second spectral range.

In some embodiments, a luminescent label refers to a fluorophore or a dye. Typically, a luminescent label comprises an aromatic or heteroaromatic compound and can be a pyrene, anthracene, naphthalene, naphthylamine, acridine, stilbene, indole, benzindole, oxazole, carbazole, thiazole, benzothiazole, benzoxazole, phenanthridine, phenoxazine, porphyrin, quinoline, ethidium, benzamide, cyanine, carbocyanine, salicylate, anthranilate, coumarin, 48luorescein, rhodamine, xanthene, or other like compound.

In some embodiments, a luminescent label comprises a dye selected from one or more of the following: 5/6-Carboxyrhodamine 6G, 5-Carboxyrhodamine 6G, 6-Carboxyrhodamine 6G, 6-TAMRA, Abberior® STAR 440SXP, Abberior® STAR 470SXP, Abberior® STAR 488, Abberior® STAR 512, Abberior® STAR 520SXP, Abberior® STAR 580, Abberior® STAR 600, Abberior® STAR 635, Abberior® STAR 635P, Abberior® STAR RED, Alexa Fluor® 350, Alexa Fluor® 405, Alexa Fluor® 430, Alexa Fluor® 480, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610-X, Alexa Fluor® 633, Alexa Fluor® 647, Alexa Fluor® 660, Alexa Fluor® 680, Alexa Fluor® 700, Alexa Fluor® 750, Alexa Fluor® 790, AMCA, ATTO 390, ATTO 425, ATTO 465, ATTO 488, ATTO 495, ATTO 514, ATTO 520, ATTO 532, ATTO 542, ATTO 550, ATTO 565, ATTO 590, ATTO 610, ATTO 620, ATTO 633, ATTO 647, ATTO 647N, ATTO 655, ATTO 665, ATTO 680, ATTO 700, ATTO 725, ATTO 740, ATTO Oxa12, ATTO Rho101, ATTO Rho11, ATTO Rho12, ATTO Rho13, ATTO Rho14, ATTO Rho3B, ATTO Rho6G, ATTO Thio12, BD Horizon™ V450, BODIPY® 493/501, BODIPY® 530/550, BODIPY® 558/568, BODIPY® 564/570, BODIPY® 576/589, BODIPY® 581/591, BODIPY® 630/650, BODIPY® 650/665, BODIPY® FL, BODIPY® FL-X, BODIPY® R6G, BODIPY® TMR, BODIPY® TR, CAL Fluor® Gold 540, CAL Fluor® Green 510, CAL Fluor® Orange 560, CAL Fluor® Red 590, CAL Fluor® Red 610, CAL Fluor® Red 615, CAL Fluor® Red 635, Cascade® Blue, CF™350, CF™405M, CF™405S, CF™488A, CF™514, CF™532, CF™543, CF™546, CF™555, CF™568, CF™594, CF™620R, CF™633, CF™633-V1, CF™640R, CF™640R-V1, CF™640R-V2, CF™660C, CF™660R, CF™680, CF™680R, CF™680R-V1, CF™750, CF™770, CF™790, Chromeo™ 642, Chromis 425N, Chromis 500N, Chromis 515N, Chromis 530N, Chromis 550A, Chromis 550C, Chromis 550Z, Chromis 560N, Chromis 570N, Chromis 577N, Chromis 600N, Chromis 630N, Chromis 645A, Chromis 645C, Chromis 645Z, Chromis 678A, Chromis 678C, Chromis 678Z, Chromis 770A, Chromis 770C, Chromis 800A, Chromis 800C, Chromis 830A, Chromis 830C, Cy®3, Cy®3.5, Cy®3B, Cy®5, Cy®5.5, Cy®7, DyLight® 350, DyLight® 405, DyLight® 415-Co1, DyLight® 425Q, DyLight® 485-LS, DyLight® 488, DyLight® 504Q, DyLight® 510-LS, DyLight® 515-LS, DyLight® 521-LS, DyLight® 530-R2, DyLight® 543Q, DyLight® 550, DyLight® 554-R0, DyLight® 554-R1, DyLight® 590-R2, DyLight® 594, DyLight® 610-B1, DyLight® 615-B2, DyLight® 633, DyLight® 633-B1, DyLight® 633-B2, DyLight® 650, DyLight® 655-B1, DyLight® 655-B2, DyLight® 655-B3, DyLight® 655-B4, DyLight® 662Q, DyLight® 675-B1, DyLight® 675-B2, DyLight® 675-B3, DyLight® 675-B4, DyLight® 679-C5, DyLight® 680, DyLight® 683Q, DyLight® 690-B1, DyLight® 690-B2, DyLight® 696Q, DyLight® 700-B1, DyLight® 700-B1, DyLight® 730-B1, DyLight® 730-B2, DyLight® 730-B3, DyLight® 730-B4, DyLight® 747, DyLight® 747-B1, DyLight® 747-B2, DyLight® 747-B3, DyLight® 747-B4, DyLight® 755, DyLight® 766Q, DyLight® 775-B2, DyLight® 775-B3, DyLight® 775-B4, DyLight® 780-B1, DyLight® 780-B2, DyLight® 780-B3, DyLight® 800, DyLight® 830-B2, Dyomics-350, Dyomics-350XL, Dyomics-360XL, Dyomics-370XL, Dyomics-375XL, Dyomics-380XL, Dyomics-390XL, Dyomics-405, Dyomics-415, Dyomics-430, Dyomics-431, Dyomics-478, Dyomics-480XL, Dyomics-481XL, Dyomics-485XL, Dyomics-490, Dyomics-495, Dyomics-505, Dyomics-510XL, Dyomics-511XL, Dyomics-520XL, Dyomics-521XL, Dyomics-530, Dyomics-547, Dyomics-547P1, Dyomics-548, Dyomics-549, Dyomics-549P1, Dyomics-550, Dyomics-554, Dyomics-555, Dyomics-556, Dyomics-560, Dyomics-590, Dyomics-591, Dyomics-594, Dyomics-601XL, Dyomics-605, Dyomics-610, Dyomics-615, Dyomics-630, Dyomics-631, Dyomics-632, Dyomics-633, Dyomics-634, Dyomics-635, Dyomics-636, Dyomics-647, Dyomics-647P1, Dyomics-648, Dyomics-648P1, Dyomics-649, Dyomics-649P1, Dyomics-650, Dyomics-651, Dyomics-652, Dyomics-654, Dyomics-675, Dyomics-676, Dyomics-677, Dyomics-678, Dyomics-679P1, Dyomics-680, Dyomics-681, Dyomics-682, Dyomics-700, Dyomics-701, Dyomics-703, Dyomics-704, Dyomics-730, Dyomics-731, Dyomics-732, Dyomics-734, Dyomics-749, Dyomics-749P1, Dyomics-750, Dyomics-751, Dyomics-752, Dyomics-754, Dyomics-776, Dyomics-777, Dyomics-778, Dyomics-780, Dyomics-781, Dyomics-782, Dyomics-800, Dyomics-831, eFluor® 450, Eosin, FITC, Fluorescein, HiLyte™ Fluor 405, HiLyte™ Fluor 488, HiLyte™ Fluor 532, HiLyte™ Fluor 555, HiLyte™ Fluor 594, HiLyte™ Fluor 647, HiLyte™ Fluor 680, HiLyte™ Fluor 750, IRDye® 680 LT, IRDye® 750, IRDye® 800CW, JOE, LightCycler® 640R, LightCycler® Red 610, LightCycler® Red 640, LightCycler® Red 670, LightCycler® Red 705, Lissamine Rhodamine B, Napthofluorescein, Oregon Green® 488, Oregon Green® 514, Pacific Blue™ Pacific Green™, Pacific Orange™, PET, PF350, PF405, PF415, PF488, PF505, PF532, PF546, PF555P, PF568, PF594, PF610, PF633P, PF647P, Quasar® 570, Quasar® 670, Quasar® 705, Rhodamine 123, Rhodamine 6G, Rhodamine B, Rhodamine Green, Rhodamine Green-X, Rhodamine Red, ROX, Seta™ 375, Seta™ 470, Seta™ 555, Seta™ 632, Seta™ 633, Seta™ 650, Seta™ 660, Seta™ 670, Seta™ 680, Seta™ 700, Seta™ 750, Seta™ 780, Seta™ APC-780, Seta™ PerCP-680, Seta™ R-PE-670, Seta™ 646, SeTau 380, SeTau 425, SeTau 647, SeTau 405, Square 635, Square 650, Square 660, Square 672, Square 680, Sulforhodamine 101, TAMRA, TET, Texas Red®, TMR, TRITC, Yakima Yellow™ Zenon®, Zy3, Zy5, Zy5.5, and Zy7.

In some aspects, the disclosure provides methods and compositions for polypeptide analysis (e.g., amino acid recognition) based on one or more luminescence properties of a luminescent label. In some embodiments, a luminescent label is identified based on luminescence lifetime, luminescence intensity, brightness, absorption spectra, emission spectra, luminescence quantum yield, or a combination of two or more thereof. In some embodiments, a plurality of types of luminescent labels can be distinguished from each other based on a difference in luminescence lifetime, luminescence intensity, brightness, absorption spectra, emission spectra, luminescence quantum yield, or combinations of two or more thereof.

In some embodiments, luminescence is detected by exposing a luminescent label to a series of separate light pulses and evaluating the timing or other properties of each photon that is emitted from the label. In some embodiments, information for a plurality of photons emitted sequentially from a label is aggregated and evaluated to identify the label and thereby identify an associated barcode site. In some embodiments, a luminescence lifetime of a label is determined from a plurality of photons that are emitted sequentially from the label, and the luminescence lifetime can be used to identify the label. In some embodiments, a luminescence intensity of a label is determined from a plurality of photons that are emitted sequentially from the label, and the luminescence intensity can be used to identify the label. In some embodiments, a luminescence lifetime and luminescence intensity of a label is determined from a plurality of photons that are emitted sequentially from the label, and the luminescence lifetime and luminescence intensity can be used to identify the label.

In some aspects of the disclosure, a single molecule is exposed to a plurality of separate light pulses and a series of emitted photons are detected and analyzed. In some embodiments, the series of emitted photons provides information about the single molecule that is present and that does not change in the mixture over the course of an experiment. However, in some embodiments, the series of emitted photons provides information about a series of different molecules that are present at different times in the mixture (e.g., as a reaction or process progresses).

In certain embodiments, a luminescent label absorbs one photon and emits one photon after a time duration. In some embodiments, the luminescence lifetime of a label can be determined or estimated by measuring the time duration. In some embodiments, the luminescence lifetime of a label can be determined or estimated by measuring a plurality of time durations for multiple pulse events and emission events. In some embodiments, the luminescence lifetime of a label can be differentiated amongst the luminescence lifetimes of a plurality of types of labels by measuring the time duration. In some embodiments, the luminescence lifetime of a label can be differentiated amongst the luminescence lifetimes of a plurality of types of labels by measuring a plurality of time durations for multiple pulse events and emission events. In certain embodiments, a label is identified or differentiated amongst a plurality of types of labels by determining or estimating the luminescence lifetime of the label. In certain embodiments, a label is identified or differentiated amongst a plurality of types of labels by differentiating the luminescence lifetime of the label amongst a plurality of the luminescence lifetimes of a plurality of types of labels.

Determination of a luminescence lifetime of a luminescent label can be performed using any suitable method (e.g., by measuring the lifetime using a suitable technique or by determining time-dependent characteristics of emission). In some embodiments, determining the luminescence lifetime of one label comprises determining the lifetime relative to another label. In some embodiments, determining the luminescence lifetime of a label comprises determining the lifetime relative to a reference. In some embodiments, determining the luminescence lifetime of a label comprises measuring the lifetime (e.g., fluorescence lifetime). In some embodiments, determining the luminescence lifetime of a label comprises determining one or more temporal characteristics that are indicative of lifetime. In some embodiments, the luminescence lifetime of a label can be determined based on a distribution of a plurality of emission events (e.g., 1, 2, 3, 4, 5, 6, 7, 8, 9, 10, 11, 12, 13, 14, 15, 16, 17, 18, 19, 20, 30, 40, 50, 60, 70, 80, 90, 100, or more emission events) occurring across one or more time-gated windows relative to an excitation pulse. For example, a luminescence lifetime of a label can be distinguished from a plurality of labels having different luminescence lifetimes based on the distribution of photon arrival times measured with respect to an excitation pulse.

It should be appreciated that a luminescence lifetime of a luminescent label is indicative of the timing of photons emitted after the label reaches an excited state and the label can be distinguished by information indicative of the timing of the photons. Some embodiments may include distinguishing a label from a plurality of labels based on the luminescence lifetime of the label by measuring times associated with photons emitted by the label. The distribution of times may provide an indication of the luminescence lifetime which may be determined from the distribution. In some embodiments, the label is distinguishable from the plurality of labels based on the distribution of times, such as by comparing the distribution of times to a reference distribution corresponding to a known label. In some embodiments, a value for the luminescence lifetime is determined from the distribution of times.

As used herein, in some embodiments, luminescence intensity refers to the number of emitted photons per unit time that are emitted by a luminescent label which is being excited by delivery of a pulsed excitation energy. In some embodiments, the luminescence intensity refers to the detected number of emitted photons per unit time that are emitted by a label which is being excited by delivery of a pulsed excitation energy, and are detected by a particular sensor or set of sensors.

As used herein, in some embodiments, brightness refers to a parameter that reports on the average emission intensity per luminescent label. Thus, in some embodiments, “emission intensity” may be used to generally refer to brightness of a composition comprising one or more labels. In some embodiments, brightness of a label is equal to the product of its quantum yield and extinction coefficient.

As used herein, in some embodiments, luminescence quantum yield refers to the fraction of excitation events at a given wavelength or within a given spectral range that lead to an emission event, and is typically less than 1. In some embodiments, the luminescence quantum yield of a luminescent label described herein is between 0 and about 0.001, between about 0.001 and about 0.01, between about 0.01 and about 0.1, between about 0.1 and about 0.5, between about 0.5 and 0.9, or between about 0.9 and 1. In some embodiments, a label is identified by determining or estimating the luminescence quantum yield.

As used herein, in some embodiments, an excitation energy is a pulse of light from a light source. In some embodiments, an excitation energy is in the visible spectrum. In some embodiments, an excitation energy is in the ultraviolet spectrum. In some embodiments, an excitation energy is in the infrared spectrum. In some embodiments, an excitation energy is at or near the absorption maximum of a luminescent label from which a plurality of emitted photons are to be detected. In certain embodiments, the excitation energy is between about 500 nm and about 700 nm (e.g., between about 500 nm and about 600 nm, between about 600 nm and about 700 nm, between about 500 nm and about 550 nm, between about 550 nm and about 600 nm, between about 600 nm and about 650 nm, or between about 650 nm and about 700 nm). In certain embodiments, an excitation energy may be monochromatic or confined to a spectral range. In some embodiments, a spectral range has a range of between about 0.1 nm and about 1 nm, between about 1 nm and about 2 nm, or between about 2 nm and about 5 nm. In some embodiments, a spectral range has a range of between about 5 nm and about 10 nm, between about 10 nm and about 50 nm, or between about 50 nm and about 100 nm.

### II. Polypeptide Analysis

In some aspects, the application provides methods of determining at least one chemical characteristic of a polypeptide by monitoring a signal for signal pulses corresponding to interactions between the polypeptide and at least one amino acid recognizer described herein, and determining at least one chemical characteristic of the polypeptide based on a characteristic pattern in the signal.

A non-limiting example of polypeptide structure analysis by detecting single molecule binding interactions during a polypeptide degradation process is illustrated in FIG. 1. An example signal trace is shown depicting different association (e.g., binding) events at times corresponding to changes in the signal. As shown, an association event between an amino acid recognizer and a terminal end of a polypeptide produces a change in magnitude of the signal that persists for a duration of time. Different association events are illustrated for different amino acids exposed at the terminal end of the polypeptide. As described herein, an amino acid that is “exposed” at the terminus of a polypeptide is an amino acid that is still attached to the polypeptide and that becomes the terminal amino acid upon removal of the prior terminal amino acid during degradation (e.g., either alone or along with one or more additional amino acids).

As generically depicted, the association events between amino acid recognizers and different types of amino acids at the terminal end of the polypeptide produce distinctive changes in the signal, referred to herein as a characteristic pattern, which may be used to determine chemical characteristics of the polypeptide. In some embodiments, a characteristic pattern corresponding to one type of terminal amino acid can be used to determine structural information for the terminal amino acid and one or more amino acids contiguous to the terminal amino acid. Accordingly, in some embodiments, a characteristic pattern corresponding to one type of terminal amino acid can be used to determine structural information for at least two (e.g., at least three, at least four, at least five, two, three, four, or between two and five) amino acids of a polypeptide.

In some embodiments, a transition from one characteristic pattern to another is indicative of amino acid cleavage. As used herein, in some embodiments, amino acid cleavage refers to the removal of at least one amino acid from a terminus of a polypeptide (e.g., the removal of at least one terminal amino acid from the polypeptide). In some embodiments, amino acid cleavage is determined by inference based on a time duration between characteristic patterns. In some embodiments, amino acid cleavage is determined by detecting a change in signal produced by association of a labeled cleaving reagent with an amino acid at the terminus of the polypeptide. As amino acids are sequentially cleaved from the terminus of the polypeptide during degradation, a series of changes in magnitude, or a series of signal pulses, is detected.

In some embodiments, signal data can be analyzed to extract signal pulse information by applying threshold levels to one or more parameters of the signal data. For example, in some embodiments, a threshold magnitude level may be applied to the signal data of a signal trace. In some embodiments, the threshold magnitude level is a minimum difference between a signal detected at a point in time and a baseline determined for a given set of data. In some embodiments, a signal pulse is assigned to each portion of the data that is indicative of a change in magnitude exceeding the threshold magnitude level and persisting for a duration of time. In some embodiments, a threshold time duration may be applied to a portion of the data that satisfies the threshold magnitude level to determine whether a signal pulse is assigned to that portion. For example, experimental artifacts may give rise to a change in magnitude exceeding the threshold magnitude level but that does not persist for a duration of time sufficient to assign a signal pulse with a desired confidence (e.g., transient association events which could be non-discriminatory for amino acid type, non-specific detection events such as diffusion into an observation region or reagent sticking within an observation region). Accordingly, in some embodiments, a signal pulse is extracted from signal data based on a threshold magnitude level and a threshold time duration.

In some embodiments, a peak in magnitude of a signal pulse is determined by averaging the magnitude detected over a duration of time that persists above the threshold magnitude level. It should be appreciated that, in some embodiments, a “signal pulse” as used herein can refer to a change in signal data that persists for a duration of time above a baseline (e.g., raw signal data), or to signal pulse information extracted therefrom (e.g., processed signal data).

In some embodiments, signal pulse information can be analyzed to identify different types of amino acids in a polypeptide based on different characteristic patterns in a series of signal pulses. For example, as shown in FIG. 1, the signal pulse information is indicative of different types of amino acids at a terminal end of a polypeptide (e.g., arginine, leucine, isoleucine, phenylalanine). By way of example, the signal pulses detected at the earliest time points provide information indicative of (at least) arginine at the terminus of the polypeptide based on a first characteristic pattern, and the signal pulses detected at the latest time points provide information indicative of at least phenylalanine at the terminus of the polypeptide based on a second characteristic pattern.

In some embodiments, each signal pulse of a characteristic pattern comprises a pulse duration corresponding to an association event between an amino acid recognizer and an amino acid ligand. In some embodiments, the pulse duration is characteristic of a dissociation rate of binding. In some embodiments, each signal pulse of a characteristic pattern is separated from another signal pulse of the characteristic pattern by an interpulse duration. In some embodiments, the interpulse duration is characteristic of an association rate of binding. In some embodiments, a change in magnitude in a signal can be determined for a signal pulse based on a difference between baseline and the peak of a signal pulse. In some embodiments, a characteristic pattern is determined based on pulse duration. In some embodiments, a characteristic pattern is determined based on pulse duration and interpulse duration. In some embodiments, a characteristic pattern is determined based on any one or more of pulse duration, interpulse duration, and change in magnitude.

Accordingly, as illustrated by FIG. 1, in some embodiments, polypeptide analysis is performed by detecting a series of signal pulses indicative of association of one or more amino acid recognizers with successive amino acids exposed at the terminus of a polypeptide in an ongoing degradation reaction. The series of signal pulses can be analyzed to determine characteristic patterns in the series of signal pulses, and the time course of characteristic patterns can be used to determine chemical characteristics throughout an amino acid sequence of the polypeptide.

As described herein, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, the mean pulse duration is between about 50 milliseconds and about 2 seconds, between about 50 milliseconds and about 500 milliseconds, or between about 500 milliseconds and about 2 seconds.

In some embodiments, different characteristic patterns corresponding to different types of amino acids in a single polypeptide may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). In some embodiments, the difference in mean pulse duration is at least 50 ms, at least 100 ms, at least 250 ms, at least 500 ms, or more. In some embodiments, the difference in mean pulse duration is between about 50 ms and about 1 s, between about 50 ms and about 500 ms, between about 50 ms and about 250 ms, between about 100 ms and about 500 ms, between about 250 ms and about 500 ms, or between about 500 ms and about 1 s. In some embodiments, the mean pulse duration of one characteristic pattern is different from the mean pulse duration of another characteristic pattern by about 10-25%, 25-50%, 50-75%, 75-100%, or more than 100%, for example by about 2-fold, 3-fold, 4-fold, 5-fold, or more. It should be appreciated that, in some embodiments, smaller differences in mean pulse duration between different characteristic patterns may require a greater number of pulse durations within each characteristic pattern to distinguish one from another with statistical confidence.

In some embodiments, a characteristic pattern generally refers to a plurality of association events between an amino acid of a polypeptide and a means for binding the amino acid (e.g., an amino acid recognition molecule). In some embodiments, a characteristic pattern comprises at least 10 association events (e.g., at least 25, at least 50, at least 75, at least 100, at least 250, at least 500, at least 1,000, or more, association events). In some embodiments, a characteristic pattern comprises between about 10 and about 1,000 association events (e.g., between about 10 and about 500 association events, between about 10 and about 250 association events, between about 10 and about 100 association events, or between about 50 and about 500 association events). In some embodiments, the plurality of association events is detected as a plurality of signal pulses.

In some embodiments, a characteristic pattern refers to a plurality of signal pulses which may be characterized by a summary statistic as described herein. In some embodiments, a characteristic pattern comprises at least 10 signal pulses (e.g., at least 25, at least 50, at least 75, at least 100, at least 250, at least 500, at least 1,000, or more, signal pulses). In some embodiments, a characteristic pattern comprises between about 10 and about 1,000 signal pulses (e.g., between about 10 and about 500 signal pulses, between about 10 and about 250 signal pulses, between about 10 and about 100 signal pulses, or between about 50 and about 500 signal pulses).

In some embodiments, a characteristic pattern refers to a plurality of association events between an amino acid recognition molecule and an amino acid of a polypeptide occurring over a time interval prior to removal of the amino acid (e.g., a cleavage event). In some embodiments, a characteristic pattern refers to a plurality of association events occurring over a time interval between two cleavage events (e.g., prior to removal of the amino acid and after removal of an amino acid previously exposed at the terminus). In some embodiments, the time interval of a characteristic pattern is between about 1 minute and about 30 minutes (e.g., between about 1 minute and about 20 minutes, between about 1 minute and 10 minutes, between about 5 minutes and about 20 minutes, between about 5 minutes and about 15 minutes, or between about 5 minutes and about 10 minutes).

In some embodiments, the series of signal pulses comprises a series of changes in magnitude of an optical signal over time. In some embodiments, the series of changes in the optical signal comprises a series of changes in luminescence produced during association events. In some embodiments, luminescence is produced by a detectable label associated with one or more reagents of a sequencing reaction. For example, in some embodiments, each of the one or more amino acid recognizers comprises a luminescent label. In some embodiments, a cleaving reagent comprises a luminescent label. Examples of luminescent labels and their use in accordance with the application are provided herein.

In some embodiments, the series of signal pulses comprises a series of changes in magnitude of an electrical signal over time. In some embodiments, the series of changes in the electrical signal comprises a series of changes in conductance produced during association events. In some embodiments, conductivity is produced by a detectable label associated with one or more reagents of a sequencing reaction. For example, in some embodiments, each of the one or more amino acid recognizers comprises a conductivity label. Examples of conductivity labels and their use in accordance with the application are provided elsewhere herein. Methods for identifying single molecules using conductivity labels have been described (see, e.g., U.S. Patent Publication No. 2017/0037462).

In some embodiments, the series of changes in conductance comprises a series of changes in conductance through a nanopore. For example, methods of evaluating receptor-ligand interactions using nanopores have been described (see, e.g., Thakur, A. K. & Movileanu, L. (2019) Nature Biotechnology 37(1)). The inventors have recognized and appreciated that such nanopores may be used to monitor polypeptide sequencing reactions in accordance with the application. Accordingly, in some embodiments, the disclosure provides methods of polypeptide analysis comprising contacting a single polypeptide molecule with one or more amino acid recognizers described herein, where the single polypeptide molecule is immobilized to a nanopore. In some embodiments, the methods further comprise detecting a series of changes in conductance through the nanopore indicative of association of the one or more amino acid recognizers with successive amino acids exposed at a terminus of the single polypeptide while the single polypeptide is being degraded.

As described herein, in some embodiments, amino acid recognizers of the disclosure may be used to determine at least one chemical characteristic of a polypeptide. In some embodiments, determining at least one chemical characteristic comprises determining the type of amino acid that is present at a terminal end of a polypeptide and/or the types of amino acids that are present at one or more positions contiguous to the amino acid at the terminal end. In some embodiments, determining the type of amino acid comprises determining the actual amino acid identity, for example by determining which of the naturally-occurring 20 amino acids is present. In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

In some embodiments, determining at least one chemical characteristic of a polypeptide comprises determining a subset of potential amino acids that can be present in the polypeptide. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be in the polypeptide (e.g., using a recognizer that binds to a specified subset of two or more amino acids).

In some embodiments, determining at least one chemical characteristic of a polypeptide comprises determining that an amino acid comprises a post-translational modification. Non-limiting examples of post-translational modifications include acetylation (e.g., acetylated lysine), ADP-ribosylation, caspase cleavage, citrullination, formylation, N-linked glycosylation (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation, sumoylation (e.g., sumoylated lysine), and ubiquitination (e.g., ubiquitinated lysine).

In some embodiments, determining at least one chemical characteristic of a polypeptide comprises determining that an amino acid comprises an arginine post-translational modification. For example, as described herein, amino acid recognizers of the disclosure are capable of distinguishing between different arginine modifications, including symmetric dimethylarginine (SDMA), asymmetric dimethylarginine (ADMA), and citrullinated arginine.

In some embodiments, determining at least one chemical characteristic of a polypeptide comprises determining that an amino acid comprises a phosphorylated side chain. For example, in some embodiments, determining at least one chemical characteristic of a polypeptide comprises determining that an amino acid comprises phosphorylated threonine (e.g., phospho-threonine). In some embodiments, determining at least one chemical characteristic of a polypeptide comprises determining that an amino acid comprises phosphorylated tyrosine (e.g., phospho-tyrosine). In some embodiments, determining at least one chemical characteristic of a polypeptide comprises determining that an amino acid comprises phosphorylated serine (e.g., phospho-serine).

In some embodiments, determining at least one chemical characteristic of a polypeptide comprises determining that an amino acid comprises a chemically modified variant, an unnatural amino acid, or a proteinogenic amino acid such as selenocysteine and pyrrolysine. Examples of unnatural amino acids include, without limitation, 2-naphthyl-alanine, statine, homoalanine, α-amino acid, β2-amino acid, β3-amino acid, γ-amino acid, 3-pyridyl-alanine, 4-fluorophenyl-alanine, cyclohexyl-alanine, N-alkyl amino acid, peptoid amino acid, homo-cysteine, penicillamine, 3-nitro-tyrosine, homo-phenyl-alanine, t-leucine, hydroxy-proline, 3-Abz, 5-F-tryptophan, and azabicyclo-[2.2.1]heptane.

In some embodiments, determining at least one chemical characteristic of a polypeptide comprises determining that an amino acid comprises an oxidative modification. For example, as described herein, amino acid recognizers of the disclosure are capable of distinguishing between oxidized methionine and its unmodified variant. In some embodiments, the oxidative modification comprises an oxidatively-damaged side chain of an amino acid. In some embodiments, the oxidatively-damaged side chain comprises a cysteine-derived product (e.g., disulfide, sulfinic acid, sulfonic acid, sulfenic acid, S-nitrosocysteine), a tyrosine-derived product (e.g., di-tyrosine, 3,4-dihydroxyphenylalanine, 3-chlorotyrosine, 3-nitrotyrosine), a histidine-derived product (e.g., 2-oxohistidine, 4-hydroxy-2-oxohistidine, di-histidine, asparagine, aspartic acid, urea), a methionine-derived product (e.g., sulfoxide, sulfone), a tryptophan-derived product (e.g., di-tryptophan, N-formylkynurenine, kynurenine, 2-oxo-tryptophan oxindolylalanine, 6-nitrotryptophan, hydroxytryptophan), a phenylalanine-derived product (e.g., meta-tyrosine, ortho-tyrosine), or a generic side-chain product (e.g., alcohol, hydroperoxide, aldehyde/ketone carbonyl). Examples of oxidatively damaged amino acids are known in the art, see, e.g., Hawkins, C. L., Davies, M. J. Detection, identification, and quantification of oxidative protein modifications. J Biol Chem. 2019 Dec. 20; 294(51):19683-19708.

In some embodiments, determining at least one chemical characteristic of a polypeptide comprises determining that an amino acid comprises a side chain characterized by one or more biochemical properties. For example, an amino acid may comprise a nonpolar aliphatic side chain, a positively charged side chain, a negatively charged side chain, a nonpolar aromatic side chain, or a polar uncharged side chain. Non-limiting examples of an amino acid comprising a nonpolar aliphatic side chain include alanine, glycine, valine, leucine, methionine, and isoleucine. Non-limiting examples of an amino acid comprising a positively charged side chain includes lysine, arginine, and histidine. Non-limiting examples of an amino acid comprising a negatively charged side chain include aspartate and glutamate. Non-limiting examples of an amino acid comprising a nonpolar, aromatic side chain include phenylalanine, tyrosine, and tryptophan. Non-limiting examples of an amino acid comprising a polar uncharged side chain include serine, threonine, cysteine, proline, asparagine, and glutamine.

In some embodiments, a protein or polypeptide can be digested into a plurality of smaller polypeptides and chemical characteristics can be determined for one or more of these smaller polypeptides. In some embodiments, a first terminus (e.g., N or C terminus) of a polypeptide is immobilized and the other terminus (e.g., the C or N terminus) is analyzed as described herein.

As used herein, sequencing a polypeptide refers to determining sequence information for a polypeptide. In some embodiments, this can involve determining the identity of each sequential amino acid for a portion (or all) of the polypeptide. However, in some embodiments, this can involve assessing the identity of a subset of amino acids within the polypeptide (e.g., and determining the relative position of one or more amino acid types without determining the identity of each amino acid in the polypeptide). However, in some embodiments, amino acid content information can be obtained from a polypeptide without directly determining the relative position of different types of amino acids in the polypeptide. The amino acid content alone may be used to infer the identity of the polypeptide that is present (e.g., by comparing the amino acid content to a database of polypeptide information and determining which polypeptide(s) have the same amino acid content).

In some embodiments, sequence information for a plurality of polypeptide products obtained from a longer polypeptide or protein (e.g., via enzymatic and/or chemical cleavage) can be analyzed to reconstruct or infer the sequence of the longer polypeptide or protein.

In some aspects, the polypeptide analysis described herein generates data indicating how a polypeptide interacts with a binding means while the polypeptide is being degraded by a cleaving means. As discussed above, the data can include a series of characteristic patterns corresponding to association events at a terminus of a polypeptide in between cleavage events at the terminus. In some embodiments, methods of polypeptide analysis described herein comprise contacting a single polypeptide molecule with a binding means and a cleaving means, where the binding means and the cleaving means are configured to achieve at least 10 association events prior to a cleavage event. In some embodiments, the means are configured to achieve the at least 10 association events between two cleavage events.

In some embodiments, a plurality of single-molecule sequencing reactions are performed in parallel in an array of sample wells. In some embodiments, an array comprises between about 10,000 and about 1,000,000 sample wells. The volume of a sample well may be between about 10⁻²¹ liters and about 10⁻¹⁵ liters, in some implementations. Because the sample well has a small volume, detection of single-molecule events may be possible as only about one polypeptide may be within a sample well at any given time. Statistically, some sample wells may not contain a single-molecule sequencing reaction and some may contain more than one single polypeptide molecule. However, an appreciable number of sample wells may each contain a single-molecule reaction (e.g., at least 30% in some embodiments), so that single-molecule analysis can be carried out in parallel for a large number of sample wells. In some embodiments, the binding means and the cleaving means are configured to achieve at least 10 association events prior to a cleavage event in at least 10% (e.g., 10-50%, more than 50%, 25-75%, at least 80%, or more) of the sample wells in which a single-molecule reaction is occurring. In some embodiments, the binding means and the cleaving means are configured to achieve at least 10 association events prior to a cleavage event for at least 50% (e.g., more than 50%, 50-75%, at least 80%, or more) of the amino acids of a polypeptide in a single-molecule reaction.

### III. Compositions and Reaction Mixtures

In some aspects, the disclosure provides compositions comprising two or more amino acid recognizers, where at least one amino acid recognizer comprises an amino acid binding protein described herein. In some embodiments, the composition comprises at least one ClpS-homologous protein described herein. In some embodiments, the composition comprises at least one UBR-homologous protein described herein. In some embodiments, the composition comprises at least one Ntaq1-homologous protein described herein. In some embodiments, the composition comprises two or more of a ClpS-homologous protein, a UBR-homologous protein, and an Ntaq1-homologous protein. In some embodiments, the composition comprises at least one ClpS-homologous protein, at least one UBR-homologous protein, and at least one Ntaq1-homologous protein.

In some embodiments, the composition further comprises at least one type of cleaving reagent. Compositions comprising amino acid recognizer and cleaving reagent may be referred to herein as a reaction mixture (e.g., a polypeptide sequencing reaction mixture). A peptidase, also referred to as a protease or proteinase, is an enzyme that catalyzes the hydrolysis of a peptide bond. Peptidases digest polypeptides into shorter fragments and may be generally classified into endopeptidases and exopeptidases, which cleave a polypeptide chain internally and terminally, respectively. In some embodiments, a cleaving reagent comprises an exopeptidase (e.g., an aminopeptidase). Examples of suitable peptidases have been described and are contemplated for use in accordance with the present disclosure. See, for example, PCT International Publication No. WO2020102741A1, filed Nov. 15, 2019, and PCT International Publication No. WO2021236983A2, filed May 20, 2021, the relevant contents of each of which are incorporated herein.

As described herein, compositions of the disclosure can be used to determine at least one chemical characteristic of a polypeptide based on a characteristic pattern. In some embodiments, polypeptide sequencing reaction conditions can be configured to achieve a time interval that allows for sufficient association events which provide a desired confidence level with a characteristic pattern. This can be achieved, for example, by configuring the reaction conditions based on various properties, including: reagent concentration, molar ratio of one reagent to another (e.g., ratio of amino acid recognition molecule to cleaving reagent, ratio of one recognizer to another, ratio of one cleaving reagent to another), number of different reagent types (e.g., the number of different types of recognizers and/or cleaving reagents, the number of recognizer types relative to the number of cleaving reagent types), cleavage activity (e.g., peptidase activity), binding properties (e.g., kinetic and/or thermodynamic binding parameters for recognition molecule binding), reagent modification (e.g., polyol and other recognizer modifications which can alter interaction dynamics), reaction mixture components (e.g., one or more components, such as pH, buffering agent, salt, divalent cation, surfactant, and other reaction mixture components described herein), temperature of the reaction, and various other parameters apparent to those skilled in the art, and combinations thereof. The reaction conditions can be configured based on one or more aspects described herein, including, for example, signal pulse information (e.g., pulse duration, interpulse duration, change in magnitude), labeling strategies (e.g., number and/or type of fluorophore, linkers with or without shielding element), surface modification (e.g., modification of sample well surface, including polypeptide immobilization), sample preparation (e.g., polypeptide fragment size, polypeptide modification for immobilization), and other aspects described herein.

In some embodiments, a polypeptide sequencing reaction in accordance with the application is performed under conditions in which recognition and cleavage of amino acids can occur simultaneously in a single reaction mixture. For example, in some embodiments, a polypeptide sequencing reaction is performed in a reaction mixture having a pH at which association events and cleavage events can occur. Accordingly, in some embodiments, a reaction mixture has a pH of between about 6.5 and about 9.0. In some embodiments, a reaction mixture has a pH of between about 7.0 and about 8.5 (e.g., between about 7.0 and about 8.0, between about 7.5 and about 8.5, between about 7.5 and about 8.0, or between about 8.0 and about 8.5).

In some embodiments, a polypeptide sequencing reaction is performed in a reaction mixture comprising one or more buffering agents. In some embodiments, a reaction mixture comprises a buffering agent in a concentration of at least 10 mM (e.g., at least 20 mM and up to 250 mM, at least 50 mM, 10-250 mM, 10-100 mM, 20-100 mM, 50-100 mM, or 100-200 mM). In some embodiments, a reaction mixture comprises a buffering agent in a concentration of between about 10 mM and about 50 mM (e.g., between about 10 mM and about 25 mM, between about 25 mM and about 50 mM, or between about 20 mM and about 40 mM). Examples of buffering agents include, without limitation, HEPES (4-(2-hydroxyethyl)-1-piperazineethanesulfonic acid), Tris(tris(hydroxymethyl)aminomethane), and MOPS (3-(N-morpholino)propanesulfonic acid).

In some embodiments, a polypeptide sequencing reaction is performed in a reaction mixture comprising salt in a concentration of at least 10 mM. In some embodiments, a reaction mixture comprises salt in a concentration of at least 10 mM (e.g., at least 20 mM, at least 50 mM, at least 100 mM, or more). In some embodiments, a reaction mixture comprises salt in a concentration of between about 10 mM and about 250 mM (e.g., between about 20 mM and about 200 mM, between about 50 mM and about 150 mM, between about 10 mM and about 50 mM, or between about 10 mM and about 100 mM). Examples of salts include, without limitation, sodium salts, potassium salts, and acetates, such as sodium chloride (NaCl), sodium acetate (NaOAc), and potassium acetate (KOAc).

Additional examples of components for use in a reaction mixture include divalent cations (e.g., Mg²⁺, Co²⁺) and surfactants (e.g., polysorbate 20). In some embodiments, a reaction mixture comprises a divalent cation in a concentration of between about 0.1 mM and about 50 mM (e.g., between about 10 mM and about 50 mM, between about 0.1 mM and about 10 mM, or between about 1 mM and about 20 mM). In some embodiments, a reaction mixture comprises a surfactant in a concentration of at least 0.01% (e.g., between about 0.01% and about 0.10%). In some embodiments, a reaction mixture comprises one or more components useful in single-molecule analysis, such as an oxygen-scavenging system (e.g., a PCA/PCD system or a Pyranose oxidase/Catalase/glucose system) and/or one or more triplet state quenchers (e.g., trolox, COT, and NBA).

In some embodiments, a polypeptide sequencing reaction is performed at a temperature at which association events and cleavage events can occur. In some embodiments, a polypeptide sequencing reaction is performed at a temperature of at least 10° C. In some embodiments, a polypeptide sequencing reaction is performed at a temperature of between about 10° C. and about 50° C. (e.g., 15-45° C., 20-40° C., at or around 25° C., at or around 30° C., at or around 35° C., at or around 37° C.). In some embodiments, a polypeptide sequencing reaction is performed at or around room temperature.

As detailed above, a real-time sequencing process as illustrated by FIG. 1 can generally involve cycles of amino acid recognition and terminal amino acid cleavage. In some embodiments, the relative occurrence of recognition and cleavage can be controlled by a concentration differential between one or more amino acid recognizers and at least one cleaving reagent. In some embodiments, the concentration differential can be optimized such that the number of signal pulses detected during recognition of an individual amino acid provides a desired confidence interval for identification. For example, if an initial sequencing reaction provides signal data with too few signal pulses between cleavage events to permit determination of characteristic patterns with a desired confidence interval, the sequencing reaction can be repeated using a decreased concentration of non-specific exopeptidase relative to recognition molecule.

In some embodiments, polypeptide analysis in accordance with the disclosure may be carried out by contacting a polypeptide with a reaction mixture comprising one or more amino acid recognizers and one or more cleaving reagents (e.g., peptidases). In some embodiments, a reaction mixture comprises an amino acid recognizer at a concentration of between about 10 nM and about 10 μM. In some embodiments, a reaction mixture comprises a cleaving reagent at a concentration of between about 500 nM and about 500 μM.

In some embodiments, a reaction mixture comprises an amino acid recognizer at a concentration of between about 100 nM and about 10 μM, between about 250 nM and about 10 μM, between about 100 nM and about 1 μM, between about 250 nM and about 1 μM, between about 250 nM and about 750 nM, or between about 500 nM and about 1 μM. In some embodiments, a reaction mixture comprises an amino acid recognizer at a concentration of about 100 nM, about 250 nM, about 500 nM, about 750 nM, or about 1 μM. In some embodiments, a reaction mixture comprises a cleaving reagent at a concentration of between about 500 nM and about 250 μM, between about 500 nM and about 100 μM, between about 1 μM and about 100 μM, between about 500 nM and about 50 μM, between about 1 μM and about 100 μM, between about 10 μM and about 200 μM, or between about 10 μM and about 100 μM. In some embodiments, a reaction mixture comprises a cleaving reagent at a concentration of about 1 μM, about 5 μM, about 10 μM, about 30 μM, about 50 μM, about 70 μM, or about 100 μM.

In some embodiments, a reaction mixture comprises an amino acid recognizer at a concentration of between about 10 nM and about 10 μM, and a cleaving reagent at a concentration of between about 500 nM and about 500 μM. In some embodiments, a reaction mixture comprises an amino acid recognizer at a concentration of between about 100 nM and about 1 μM, and a cleaving reagent at a concentration of between about 1 μM and about 100 μM. In some embodiments, a reaction mixture comprises an amino acid recognizer at a concentration of between about 250 nM and about 1 μM, and a cleaving reagent at a concentration of between about 10 μM and about 100 μM. In some embodiments, a reaction mixture comprises an amino acid recognizer at a concentration of about 500 nM, and a cleaving reagent at a concentration of between about 25 μM and about 75 μM. In some embodiments, the concentration of an amino acid recognizer and/or the concentration of a cleaving reagent in a reaction mixture is as described elsewhere herein.

In some embodiments, a reaction mixture comprises an amino acid recognizer and a cleaving reagent in a molar ratio of about 500:1, about 400:1, about 300:1, about 200:1, about 100:1, about 75:1, about 50:1, about 25:1, about 10:1, about 5:1, about 2:1, or about 1:1. In some embodiments, a reaction mixture comprises an amino acid recognizer and a cleaving reagent in a molar ratio of between about 10:1 and about 200:1. In some embodiments, a reaction mixture comprises an amino acid recognizer and a cleaving reagent in a molar ratio of between about 50:1 and about 150:1. In some embodiments, the molar ratio of an amino acid recognizer to a cleaving reagent in a reaction mixture is between about 1:1,000 and about 1:1 or between about 1:1 and about 100:1 (e.g., 1:1,000, about 1:500, about 1:200, about 1:100, about 1:10, about 1:5, about 1:2, about 1:1, about 5:1, about 10:1, about 50:1, about 100:1). In some embodiments, the molar ratio of an amino acid recognizer to a cleaving reagent in a reaction mixture is between about 1:100 and about 1:1 or between about 1:1 and about 10:1. In some embodiments, the molar ratio of an amino acid recognizer to a cleaving reagent in a reaction mixture is as described elsewhere herein.

In some embodiments, a reaction mixture comprises one or more amino acid recognizer and one or more cleaving reagents. In some embodiments, a reaction mixture comprises at least three amino acid recognizers and at least one cleaving reagent. In some embodiments, the reaction mixture comprises two or more cleaving reagents. In some embodiments, the reaction mixture comprises at least one and up to ten cleaving reagents (e.g., 1-3 cleaving reagents, 2-10 cleaving reagents, 1-5 cleaving reagents, 3-10 cleaving reagents). In some embodiments, the reaction mixture comprises at least three and up to thirty amino acid recognizers (e.g., between 3 and 25, between 3 and 20, between 3 and 10, between 3 and 5, between 5 and 30, between 5 and 20, between 5 and 10, or between 10 and 20, amino acid recognizers). In some embodiments, the one or more amino acid recognizers include at least one amino acid binding protein selected from Table 1.

In some embodiments, a reaction mixture comprises more than one amino acid recognizer and/or more than one cleaving reagent. In some embodiments, a reaction mixture described as comprising more than one amino acid recognizer (or cleaving reagent) refers to the mixture as having more than one type of amino acid recognizer (or cleaving reagent). For example, in some embodiments, a reaction mixture comprises two or more amino acid binding proteins, where the two or more amino acid binding proteins refer to two or more types of amino acid binding proteins. In some embodiments, one type of amino acid binding protein has an amino acid sequence that is different from another type of amino acid binding protein in the reaction mixture. In some embodiments, one type of amino acid binding protein has a label that is different from a label of another type of amino acid binding protein in the reaction mixture. In some embodiments, one type of amino acid binding protein associates with (e.g., binds to) an amino acid that is different from an amino acid with which another type of amino acid binding protein in the reaction mixture associates. In some embodiments, one type of amino acid binding protein associates with (e.g., binds to) a subset of amino acids that is different from a subset of amino acids with which another type of amino acid binding protein in the reaction mixture associates.

### IV. Devices and Systems

Methods in accordance with the disclosure, in some aspects, may be performed using a system that permits single-molecule analysis. The system may include an integrated device and an instrument configured to interface with the integrated device. The integrated device may include an array of pixels, where individual pixels include a sample well and at least one photodetector. The sample wells of the integrated device may be formed on or through a surface of the integrated device and be configured to receive a sample placed on the surface of the integrated device. Collectively, the sample wells may be considered as an array of sample wells. The plurality of sample wells may have a suitable size and shape such that at least a portion of the sample wells receive a single sample (e.g., a single molecule, such as a polypeptide). In some embodiments, the number of samples within a sample well may be distributed among the sample wells of the integrated device such that some sample wells contain one sample while others contain zero, two or more samples.

Excitation light is provided to the integrated device from one or more light source external to the integrated device. Optical components of the integrated device may receive the excitation light from the light source and direct the light towards the array of sample wells of the integrated device and illuminate an illumination region within the sample well. In some embodiments, a sample well may have a configuration that allows for the sample to be retained in proximity to a surface of the sample well, which may ease delivery of excitation light to the sample and detection of emission light from the sample. A sample positioned within the illumination region may emit emission light in response to being illuminated by the excitation light. For example, the sample may be labeled with a fluorescent label, which emits light in response to achieving an excited state through the illumination of excitation light. Emission light emitted by a sample may then be detected by one or more photodetectors within a pixel corresponding to the sample well with the sample being analyzed. When performed across the array of sample wells, which may range in number between approximately 10,000 pixels to 1,000,000 pixels according to some embodiments, multiple samples can be analyzed in parallel.

The integrated device may include an optical system for receiving excitation light and directing the excitation light among the sample well array. The optical system may include one or more grating couplers configured to couple excitation light to other optical components of the integrated device and direct the excitation light to the other optical components. For example, the optical system may include optical components that direct the excitation light from the grating coupler(s) towards the sample well array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the integrated device by improving the uniformity of excitation light received by sample wells of the integrated device. Examples of suitable components, e.g., for coupling excitation light to a sample well and/or directing emission light to a photodetector, to include in an integrated device are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated by reference in their entirety. Examples of suitable grating couplers and waveguides that may be implemented in the integrated device are described in U.S. patent application Ser. No. 15/844,403, filed Dec. 15, 2017, titled “OPTICAL COUPLER AND WAVEGUIDE SYSTEM,” which is incorporated by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the integrated device, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” and U.S. Provisional Patent Application No. 63/124,655, filed Dec. 11, 2020, titled “INTEGRATED CIRCUIT WITH IMPROVED CHARGE TRANSFER EFFICIENCY AND ASSOCIATED TECHNIQUES,” both of which are incorporated by reference in their entirety.

Components located off of the integrated device may be used to position and align an excitation source to the integrated device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated by reference in its entirety.

The photodetector(s) positioned with individual pixels of the integrated device may be configured and positioned to detect emission light from the pixel's corresponding sample well. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated by reference in its entirety. In some embodiments, a sample well and its respective photodetector(s) may be aligned along a common axis. In this manner, the photodetector(s) may overlap with the sample well within the pixel.

Characteristics of the detected emission light may provide an indication for identifying the label associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, such characteristics can be any one or a combination of two or more of luminescence lifetime, luminescence intensity, brightness, absorption spectra, emission spectra, luminescence quantum yield, wavelength (e.g., peak wavelength), and signal characteristics (e.g., pulse duration, interpulse durations, change in signal magnitude).

In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the integrated device, and the distribution of arrival times may provide an indication of a timing characteristic of the sample's emission light (e.g., a proxy for luminescence lifetime). In some embodiments, the one or more photodetectors provide an indication of the probability of emission light emitted by the label (e.g., luminescence intensity). In some embodiments, a plurality of photodetectors may be sized and arranged to capture a spatial distribution of the emission light. Output signals from the one or more photodetectors may then be used to distinguish a label from among a plurality of labels, where the plurality of labels may be used to identify a sample within the sample. In some embodiments, a sample may be excited by multiple excitation energies, and emission light and/or timing characteristics of the emission light emitted by the sample in response to the multiple excitation energies may distinguish a label from a plurality of labels.

In operation, parallel analyses of samples within the sample wells are carried out by exciting some or all of the samples within the wells using excitation light and detecting signals from sample emission with the photodetectors. Emission light from a sample may be detected by a corresponding photodetector and converted to at least one electrical signal. The electrical signals may be transmitted along conducting lines in the circuitry of the integrated device, which may be connected to an instrument interfaced with the integrated device. The electrical signals may be subsequently processed and/or analyzed. Processing or analyzing of electrical signals may occur on a suitable computing device either located on or off the instrument.

The instrument may include a user interface for controlling operation of the instrument and/or the integrated device. The user interface may be configured to allow a user to input information into the instrument, such as commands and/or settings used to control the functioning of the instrument. In some embodiments, the user interface may include buttons, switches, dials, and a microphone for voice commands. The user interface may allow a user to receive feedback on the performance of the instrument and/or integrated device, such as proper alignment and/or information obtained by readout signals from the photodetectors on the integrated device. In some embodiments, the user interface may provide feedback using a speaker to provide audible feedback. In some embodiments, the user interface may include indicator lights and/or a display screen for providing visual feedback to a user.

In some embodiments, the instrument may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the integrated device, and/or data generated from the readout signals of the photodetector.

In some embodiments, the instrument may include a processing device configured to analyze data received from one or more photodetectors of the integrated device and/or transmit control signals to the excitation source(s). In some embodiments, the processing device may comprise a general purpose processor, a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the integrated device.

According to some embodiments, the instrument that is configured to analyze samples based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region can be less complex to operate and maintain, more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference.

In some embodiments, different numbers of fluorophores of the same type may be linked to different reagents in a sample, so that each reagent may be identified based on luminescence intensity. For example, two fluorophores may be linked to a first labeled recognition molecule and four or more fluorophores may be linked to a second labeled recognition molecule. Because of the different numbers of fluorophores, there may be different excitation and fluorophore emission probabilities associated with the different recognition molecules. For example, there may be more emission events for the second labeled recognition molecule during a signal accumulation interval, so that the apparent intensity of the bins is significantly higher than for the first labeled recognition molecule.

The inventors have recognized and appreciated that distinguishing biological or chemical samples based on fluorophore decay rates and/or fluorophore intensities may enable a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each sample well to detect emission from different fluorophores. The phrase “characteristic wavelength” or “wavelength” is used to refer to a central or predominant wavelength within a limited bandwidth of radiation (e.g., a central or peak wavelength within a 20 nm bandwidth output by a pulsed optical source). In some cases, “characteristic wavelength” or “wavelength” may be used to refer to a peak wavelength within a total bandwidth of radiation output by a source.

According to an aspect of the present disclosure, an exemplary integrated device may be configured to perform single-molecule analysis in combination with an instrument as described above. It should be appreciated that the exemplary integrated device described herein is intended to be illustrative and that other integrated device configurations may be configured to perform any or all techniques described herein.

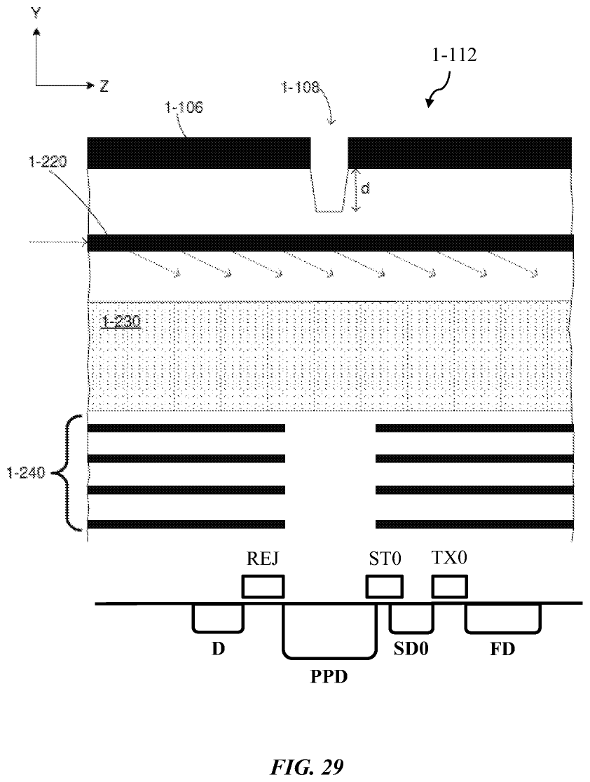

FIG. 29 illustrates a cross-sectional view of a pixel 1-112 of an integrated device 1-102. Pixel 1-112 includes a photodetection region, which may be a pinned photodiode (PPD), and a charge storage region, which may be a storage diode (SD0). In some embodiments, a photodetection region and charge storage regions may be formed in semiconductor material of a pixel by doping regions of the semiconductor material. For example, the photodetection region and charge storage regions can be formed using a same conductivity type (e.g., n-type doping or p-type doping).

During operation of pixel 1-112, excitation light may illuminate sample well 1-108 causing incident photons, including fluorescence emissions from a sample, to flow along the optical axis to photodetection region PPD. As shown in FIG. 29, pixel 1-112 may include a waveguide 1-220 configured to optically (e.g., evanescently) couple excitation light from a grating coupler of the integrated device (not shown) to the sample well 1-108. In response, a sample in the sample well 1-108 may emit fluorescent light toward photodetection region PPD. In some embodiments, pixel 1-112 may also include one or more photonic structures 1-230, which may include one or more optical rejection structures such as a spectral filter, a polarization filter, and/or a spatial filter. For example, the photonic structures 1-230 may be configured to reduce the amount of excitation light that reaches the photodetection region PPD and/or increase the amount of fluorescent emissions that reach the photodetection region PPD. Also shown in pixel 1-112, pixel 1-112 may include one or more metal layers 1-240, which may be configured as a filter and/or may carry control signals from a control circuit configured to control transfer gates, as described further herein.

In some embodiments, pixel 1-112 may include one or more transfer gates configured to control operation of pixel 1-112 by applying an electrical bias to one or more semiconductor regions of pixel 1-112 in response to one or more control signals. For example, when transfer gate ST0 induces a first electrical bias at the semiconductor region between photodetection region PPD and storage region SD0, a transfer path (e.g., charge transfer channel) may be formed in the semiconductor region. Charge carriers (e.g., photoelectrons) generated in photodetection region PPD by the incident photons may flow along the transfer path to storage region SD0. In some embodiments, the first electrical bias may be applied during a collection period during which charge carriers from the sample are selectively directed to storage region SD0. Alternatively, when transfer gate ST0 provides a second electrical bias at the semiconductor region between photodetection region PPD and storage region SD0, charge carriers from photodetection region PPD may be blocked from reaching storage region SD0 along the transfer path. In some embodiments, drain gate REJ may provide a channel to drain D to draw noise charge carriers generated in photodetection region PPD by the excitation light away from photodetection region PPD and storage region SD0, such as during a rejection period before fluorescent emission photons from the sample reach photodetection region PPD. In some embodiments, during a readout period, transfer gate ST0 may provide the second electrical bias and transfer gate TX0 may provide an electrical bias to cause charge carriers stored in storage region SD0 to flow to the readout region, which may be a floating diffusion (FD) region, for processing.

It should be appreciated that, in accordance with various embodiments, transfer gates described herein may include semiconductor material(s) and/or metal, and may include a gate of a field effect transistor (FET), a base of a bipolar junction transistor (BJT), and/or the like.

In some embodiments, operation of pixel 1-112 may include one or more collection sequences, each collection sequence including one or more rejection (e.g., drain) periods and one or more collection periods. In one example, a collection sequence performed in accordance with one or more pulses of an excitation light source may begin with a rejection period, such as to discard charge carriers generated in pixel 1-112 (e.g., in photodetection region PD) responsive to excitation photons from the light source. For instance, the excitation photons may arrive at pixel 1-112 prior to the arrival of fluorescence emission photons from the sample well. Transfer gates for the charge storage regions may be biased to have low conductivity in the charge transfer channels coupling the charge storage regions to the photodetection region, blocking transfer and accumulation of charge carriers in the charge storage regions. A drain gate for the drain region may be biased to have high conductivity in a drain channel between the photodetection region and the drain region, facilitating draining of charge carriers from the photodetection region to the drain region. Transfer gates for any charge storage regions coupled to the photodetection region may be biased to have low conductivity between the photodetection region and the charge storage regions, such that charge carriers are not transferred to or accumulated in the charge storage regions during the rejection period.

Following the rejection period, a collection period may occur in which charge carriers generated responsive to the incident photons are transferred to one or more charge storage regions. During the collection period, the incident photons may include fluorescent emission photons, resulting in accumulation of fluorescent emission charge carriers in the charge storage region(s). For instance, a transfer gate for one of the charge storage regions may be biased to have high conductivity between the photodetection region and the charge storage region, facilitating accumulation of charge carriers in the charge storage region. Any drain gates coupled to the photodetection region may be biased to have low conductivity between the photodetection region and the drain region such that charge carriers are not discarded during the collection period.

Some embodiments may include multiple rejection and/or collection periods in a collection sequence, such as a second rejection period and second collection period following a first rejection period and a collection period, where each pair of rejection and collection periods is conducted in response to a pulse of excitation light. In one example, charge carriers generated in the photodetection region during each collection period of a collection sequence (e.g., in response to a plurality of pulses of excitation light) may be aggregated in a single charge storage region. In some embodiments, charge carriers aggregated in the charge storage region may be read out for processing prior to the next collection sequence. Alternatively or additionally, in some embodiments, charge carriers aggregated in a first charge storage region during a first collection sequence may be transferred to a second charge storage region sequentially coupled to the first charge storage region and read out simultaneously with the next collection sequence. In some embodiments, a processing circuit configured to read out charge carriers from one or more pixels may be configured to determine one or more of luminescence intensity information, luminescence lifetime information, luminescence spectral information, and/or any other mode of luminescence information associated with performing techniques described herein.

In some embodiments, a first collection sequence may include transferring, to a charge storage region at a first time following each excitation pulse, charge carriers generated in the photodetection response in response to the excitation pulse, and a second collection sequence may include transferring, to the charge storage region at a second time following each excitation pulse, charge carriers generated in the photodetection response in response to the excitation pulse. For example, the number of charge carriers aggregated after the first and second times may indicate luminance lifetime information of the received light.

As described further herein, pixels of an integrated device may be controlled to perform one or more collection sequences using one or more control signals from a control circuit of the integrated circuit, such as by providing the control signal(s) to drain and/or transfer gates of the pixel(s) of the integrated circuit. In some embodiments, charge carriers may be read out from the FD region of each pixel during a readout pixel associated with each pixel and/or a row or column of pixels for processing. In some embodiments, FD regions of the pixels may be read out using correlated double sampling (CDS) techniques.

### V. Sequence Information

TABLE 1
Non-limiting example sequences of amino acid binding proteins.

| | SEQ | |
|---|---|---|
| | ID | |
| Name | NO. | Sequence |
| PS557 | 1 | MPTAASATESAIEDTPAPARPEVDGRTKPKRQPRYHVVLWNDDDHTYQYVVVMLQSLFGH |
| | | PPERGYRLAKEVDTQGRVIVLTTTREHAELKRDQIHAFGYDRLLARSKGSMKASIEAEE |
| PS621 | 2 | MHSKFSHAGRICGAKFKVGEPIYRCKECSFDDTCVLCVNCFNPKDHTGHHVYTTICTEFN |
| | | NGICDCGDKEAWNHTLFCKAEEG |
| Ntaq1sf | 3 | MNGLSAQHERIAPARHECVYTSCYCEENVWKLCEHIKTSKRCPLGDVYAVFISNERKMVP |
| | | IWKQKSGRGEEPVIWDYHVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKT |
| PS579 | 4 | MSDSPVDLKPKPKVKPKLERPKLYKVMLLNDDYTTAFFVTKVLKAVFRMSEDTGRRVMMT |
| | | AHRFGSAVVVVCERDIAETKAKEATDLGKEAGFPLMFTTEPEE |
| PS580 | 5 | MSDSPVDLKPKPKVKPKLERPKLYKVMLLNDDYTTMRFVTLVLKAVFRMSEDTGRRVMMT |
| | | AHRFGSAVVVVCERDIAETKAKEATDLGKEAGFPLMFTTEPEE |
| PS581 | 6 | MLSATRRALQLFHSLFPIPRMGDSAAKIVSPQEALPGRKEPLVVAAKHHVNGNRTVEPFP |
| | | EGTQMAVFGMGCFWGAERKFWTLKGVYSTQVGFAGGYTPNPTYKEVCSGKTGHAEVVRVV |
| | | FQPEHISFEELLKVFWENHDPTQGMRQGNDHGSQYRSAIYPTSAEHVGAALKSKEDYQKV |
| | | LSEHGFGLITTDIREGQTFYYAEDYHQQYLSKDPDGYCGLGGTGVSCPLGIKK |
| PS582 | 7 | MLSATRRALQLFHSLFPIPRMGDSAAKIVSPQEALPGRKEPLVVAAKHHVNGNRTVEPFP |
| | | EGTQMAVFGMGSFWGAERKFWTLKGVYSTQVGFAGGYTPNPTYKEVCSGKTGHAEVVRVV |
| | | FQPEHISFEELLKVFWENHDPTQGMRQGNDHGSQYRSAIYPTSAEHVGAALKSKEDYQKV |
| | | LSEHGFGLITTDIREGQTFYYAEDYHQQYLSKDPDGYCGLGGTGVSCPLGIKK |
| PS585 | 8 | MAFPARGKTAPKNEVRRQPPYNVILLNDDDTTYRYVIEMLQKIFGFPPEKGFQIAEEVDR |
| | | TGRVILLTTSKEHAELKQDQVHSYGPDPYLGRPCSGSMTCVIEPAV |
| PS586 | 9 | MAFPARGKTAPKNEVRRQPPYNVILLDDDDHTYRYVIEMLQKIFGFPPEKGFQIAEEVDR |
| | | TGRVILLTTSKEHAELKQDQVHSYGPDPYLGRPCSGSMTCVIEPAV |
| PS587 | 10 | MAFPARGKTAPKNEVRRQPPYNVILLKDDDHTYRYVIEMLQKIFGFPPEKGFQIAEEVDR |
| | | TGRVILLTTSKEHAELKQDQVHSYGPDPYLGRPCSGSMTCVIEPAV |
| PS588 | 11 | MAFPARGKTAPKNEVRRQPPYNVILLNKDDHTYRYVIEMLQKIFGFPPEKGFQIAEEVDR |
| | | TGRVILLTTSKEHAELKQDQVHSYGPDPYLGRPCSGSMTCVIEPAV |
| PS589 | 12 | MAFPARGKTAPKNEVRRQPPYNVILLNDDDHTYRYVIEMLQKIFGFPPEKGFQIAEEVHR |
| | | TGRVILLTTSKEHAELKQDQVHSYGPDPYLGRPCSGSMTCVIEPAV |
| PS590 | 13 | MAFPARGKTAPKNEVRRQPPYNVILLNDDNHTYRYVIEMLQKIFGFPPEKGFQIAEEVDR |
| | | TGRVILLTTSKEHAELKQDQVHSYGPDPYLGRPCSGSMTCVIEPAV |
| PS591 | 14 | MGSVHKHTGRNCGRKFKIGEPLYRCHECGCDDTCVLCIHCFNPKDHVKHHVCTDICTEFT |
| | | SGICDCGDEEAWNSPLHCKAEEQ |
| PS594 | 15 | MTSLNIMGRKFILERAKRNDNIEEIYTSAYVSLPSSTDTRLPHFKAKEEDCDVYEEGTNL |
| | | VGKNAKYTYRSLGRHLDFLRPGLRFGGSQSSKYTYYTVEVKIDTVNLPLYKDSRSLDPHV |
| | | TGTFTIKNLTPVLDKVVTLFEGYVINYNQFPLCSLHWPAEETLDPYMAQRESDCSHWKRF |
| | | GHFGSDNWSLTERNFGQYNHESAEFMNQRYIYLKWKERFLLDDEEQENQMLDDNHHLEGA |
| | | SFEGFYYVCLDQLTGSVEGYYYHPACELFQKLELVPTNCDALNTYSSGFEIA |
| PS595 | 16 | MGSSHHHHHHHHHHSSGLVPRGSHMQIFVKTLTGKTITLEVEPSDTIENVKAKIQDKEGI |
| | | PPDQQRLIFAGKQLEDGRTLSDYNIQKESTLHLVLRLRGGMASVVEYKGLKAGYYCGYCE |
| | | SREGKTSCGMWAHSMTVQDYQDLIDRGWRRSGKYVYKPVMDQTCCPQYTIRCHPLQFQPS |
| | | KSHKKVLKKMLKFLAKGEISKGNCEDEPMDSTVEDAVDGDFALINKLDIKCDLKTLSDLK |
| | | GSIESEEKEKEKSIKKEGSKEFIHPQSIEEKLGSGEPSHPIKVHIGPKPGKGADLSKPPC |
| | | RKAREMRKERQRLKRMQQASAAASEAQGQPVCLLPKAKSNQPKSLEDLIFQSLPENASHK |
| | | LEVRLVPASFEDPEFNSSFNQSFSLYTKYQVAIHQEAPEICEKSEFTRFLCSSPLEAEHP |
| | | ADGPECGYGSFHQQYWLDGKIIAVGVLDILPYCVSSVYLYYDPDYSFLSLGVYSALREIA |
| | | FTRQLHEKTSQLSYYYMGFYIHSCPKMRYKGQYRPSDLLCPETYVWVPIEQCLPSLDNSK |
| | | YCRFNQDPEAEDEGRSKELDRLRVFHRRSAMPYGVYKNHQEDPSEEAGVLEYANLVGQKC |
| | | SERMLLFRH |
| PS630 | 17 | MSEPMTLPAIPQPRLKERTQRQPPYNVILLNDDDKSYEYVAAMLQVLFGYPPEKGYQMAK |
| | | EVDSTGRVILLTTTREHAELKQEQIHAFGPDPNQARNSGSMKAVIEPAV |
| PS631 | 18 | MSEPMTLPAIPQPRLKERTQRQPPYNVILLNDDDKSYEYVIAMLQVLFGYPPEKGYQMAK |
| | | EVDSTGRVILLTTTREHAELKQEQIHAFGPDPNQARNSGSMKAVIEPAV |
| PS632 | 19 | MSEPMTLPAIPQPRLKERTQRQPPYNVIILNDDDKSFEYVAAMLQVLFGYPPEKGYQMAK |
| | | EIDSTGRVIMLTTTREHAELKQEQIHAFGPDPNQARNSGSMKAVIEPAV |
| PS633 | 20 | MSEPMTLPAIPQPRLKERTQRQPPYNVIILNDDDKSFEYVAALLQVLFGYPPEKGYQMAK |
| | | EIDSTGRVIMLTTTREHAELKQEQIHAFGPDPNQARNSGSMKAVIEPAV |
| PS634 | 21 | MSEPMTLPAIPQPRLKERTQRQPPYNVIVLNDDDKSFEYVAAMLQVLFGYPPEKGYQVAK |
| | | EIDSTGRVITLTTTREHAELKQEQIHAFGPDPNQARNSGSMKAVIEPAV |
| PS635 | 22 | MPTAASATESAIEDTPAPARPEVDGRTKPKRQPRYHVVQWNDDRHTYQYTVVMFQSLFGH |
| | | PPERGYRLAKESDTQGRIIVLTTTREHAELKRDQIHAFGYDRLLARDKGSYKASIEAEE |
| PS636 | 23 | HHHHHHHHHHDYDIPTTENLYFQGMPTAASATESAIEDTPAPARPEVDGRTKPKRQPRYH |
| | | VVLWNDDDHTYQYVVVMLQSLFGHPPERGYRLAKEVDTQGRVIVLTTTREHAELKRDQIH |
| | | AFGYDRLLARSKGSMKASIEAEE |
| PS642 | 24 | MPTAASATESAIEDTPAPARPEVDGRTKPKRQPRYHVVQWNDDRHTYQYTVVMFQSLFGH |
| | | PPERGYRLAKESDTQGRIIVLTTTREHAELKRDQIHAFGYDPLQSGDKGSYKASIEAEE |
| PS643 | 25 | MPTAASATESAIEDTPAPARPEVDGRTKPKRQPRYHVVAWNDDRHTYQYTVVMFQSLFGH |
| | | PPERGYRLAKEQDTQGRIIVLTTTREHAELKRDQIHAFGYDPLQSGDKGSYKASIEAEE |
| PS644 | 26 | MPTAASATESAIEDTPAPARPEVDGRTKPKRQPRYHVVAWNDDRHTYQYTVVMFQSLFGH |
| | | PPERGYRLAKEQDTQGRIIVLTTTREHAELKRDQIHAFGYDPLQSGDKGSYKASIEAEE |
| PS645 | 27 | MPTAASATESAIEDTPAPARPEVDGRTKPKRQPRYHVVSWNDDKHTYQWTVVMFQSLFGH |
| | | PPERGYRLAKERDTQGRIIVLTTTREHAELKRDQIHAFGYDPLQSGDKGSMKASIEAEE |
| PS646 | 28 | MKMYNIPTPTMAQVIMVDDPITTTEFVISALRDFFDKSLEEAKALTSSIHRDGEGVCGVY |
| | | PYDIARHRAAWVRDKAKALEFPLKLLVEEIK |
| PS647 | 29 | MKMYNIPTPTMAQVIMVDDPINTYEFTISALRDFFDKSLEEAKALASSIDRDGEGVCGVY |
| | | PYDIARHRAAWVRDKAKALEFPEKLLVEEIK |
| PS648 | 30 | MKMYNIPTPTMAQVIMVDDPINTKEFTISALRDFFDKSLEEAKALASSIDRDGEGVCGVY |
| | | PYDIARHRAAWVRDKAKALEFPEKLLVEEIK |
| PS649 | 31 | MKMYNIPTPTMAQVIRVDDPSMTNEFGISALRDFFDKSLEEAKALASSIDRDGEGVCGVY |
| | | PYDIARHRAAWVRDKAKALEFPSKLLVEEIK |
| PS650 | 32 | MKMYNIPTPTMAQVIRVDDPSMTYEFGISALRDFFDKSLEEAKALASSIDRDGEGVCGVY |
| | | PYDIARHRAAWVRDKAKALEFPSKLLVEEIK |
| PS657 | 33 | MPQERQQVTRKHYPNYKVIFLNSDFYTFQHLVALMMKYIPNMTSDRAWEISNQIHYEGQA |
| | | IVWVGPQEQAELYHEQFLRAGLTMAPLEPE |
| PS658 | 34 | MTSTLRARPARDTDLQHRPYPHYRIITLDDDVMTFQHMANSYVTFLPGMTRDQMWAMSQQ |
| | | DDGEGSMVVWTGPQEQAELYHVQLGNHGQTNIPLEPV |
| PS659 | 35 | MTSTLRARPARDTDLQHRPYPHYRIIVLDDDVMTFQHMANSFVTFLPGMTRDQMWAMSQQ |
| | | DEGEGSMVVWTGPQEQAELYHVQLGNHGQTNIPLEPV |
| PS660 | 36 | MTSTLRARPARDTDLQHRPYPHYRIIVLDDDVMTFQHLANSFVTFLPGMTRDQMWAMSQQ |
| | | DDGEGSMVVWTGPQEQAELYHVQLGNHGQTNIPLEPV |
| PS661 | 37 | MTSTLRARPARDTDLQHRPYPHYRIIVLDDDVMTFQHMANSFVTFLPGMTRDQMWAMSQQ |
| | | DDGEGSMVVWTGPQEQAELYHVQLGNHGQTNIPLEPV |
| PS662 | 38 | MTSTLRARPARDTDLQHRPYPHYRIILLDSDVITFQLTANAFVTFLPGMTRDQMWAKIQQ |
| | | SDGEGSCVVWTGPQEQAELYHVQLGNQGLTEIPLEPV |
| PS663 | 39 | MVSIGAATVACAEGRPIFSGYFDWLAAMPETVTVPRTRLRPKTERPKLHKVIEVNQDYIP |
| | | WEFWVTFFKGEFHMSEDQAQRKMIAGDRRGVYVVAVFTRDVAETKATRFSDHGRAKGYPT |
| | | QMTTEPEE |
| PS664 | 40 | MGQTVEKPRVEGPGTGLGGSWRVITRNNDHYTRDHWARTIARFIPGVSLERAHEWSKVIH |
| | | TTGRKVVYTGHKEAAEHYWQQFKGSGLESMPLEQG |
| PS665 | 41 | MTLSVALGPDTQESTQTGTAVSTDTLTAPDIPWNLVDWNDPVNLMSYISYVFQSYFGYSE |
| | | TKANKLMMEQDKKGRSIVAHGSKEQVEQHAVALHGYGNWATVEKATGGNSGGGKSGGPGK |
| | | GKGKRG |
| PS666 | 42 | MSGTVVESKPRNSTQLAPRWKVIYHDNPVTTFDFTTGMFRRVFAKPPGEARRMTREAHDT |
| | | GSVLVDVLALEQAEFRRDQMHSLARAEGFPQTLTLEPAD |
| PS667 | 43 | MSGTVVESKPRNSTQLAPRWKVIYHDQPVTTFDFTTGLFRRVFAKPPGEARRMTREAHDT |
| | | GSVLVDVLALEQAEFRRDQMHSLARAEGFPQTLTLEPAD |
| PS668 | 44 | MSDSPVDLKPKPKVKPKLERPSMYKVITVNDDYTPMEFTIDHLQKFFSYDVERATQLMLA |
| | | SDYQGKAICGVFTAEVAETKVAMMNKSARENEHPELCTLEKAE |
| PS669 | 45 | MSDSPVDLKPKPKVKPKLERPSMYKVITVNDDYTPMEFTIDHLQKFFSYDVERATQLMLA |
| | | SEYQGKAICGVFTAEVAETKVAMMNKSARENEHPELCTLEKAE |
| PS670 | 46 | MHSKFNHAGRICGAKHRVGEPMYRCKECSFDDTCTLCVNCFNPKDHVGHHVYTSICTEFK |
| | | NGICDCGDKEAWNHELNCKGAED |
| PS671 | 47 | MHSKFNHAGRICGAKFRVGEPLYRCKECSFDDTCVLCVNCFNPKDHVGHHVYTSICTEFL |
| | | NGICDCGDKEAWNHELNCKGAED |
| PS672 | 48 | MHSKFNHAGRICGAKFRVGEPLYKCKECSFDDTCVLCVNCFNPKDHVGHHVYTMICTEFL |
| | | NGICDCGDKEAWNHELNCKGAED |
| PS673 | 49 | MAFPARGKTAPKNEVRRQPPYNVIMLNDDDHTWRYAMELFQKIFGFPPEKGFQIVEEMDR |
| | | TGRVILLTTSKEHAELKQDQMHSYGPDPYLGRPCSGSMTCVIEPAV |
| PS674 | 50 | MAFPARGKTAPKNEVRRQPPYNVIILNDDDHTWRYLMEMFQKIFGFPPEKGFQIIEEIDR |
| | | TGRAILLTTSKEHAELKQDQLHSYGPDPYLGRPCSGSMTVVIEPAV |
| PS675 | 51 | MAFPARGKTAPKNEVRRQPPYNVILLNDDDHTWRYIMEMFQKIFGFPPEKGFQITEEIDR |
| | | TGRAILLTTSKEHAELKQDQTHSYGPDPYLGRPCSGSMTMVIEPAV |
| PS676 | 52 | MAFPARGKTAPKNEVRRQPPYNVIILNDDDMTWRYLMEAFQKIFGFPPEKGFQIIEEIDR |
| | | TGRAILLTTSKEHAELKQDQMHSYGPDPYLGRPCSGSMTMVIEPAV |
| PS677 | 53 | MSGTVVESKPRNSTQLAPRWKVIMHDQPVITFDFTLGMFRRVFAKPPGEARRITREAHDT |
| | | GSVLVDVLALEQAEFRRDQMHSLARAEGFPLTMTLEPAD |
| PS678 | 54 | MAFPARGKTAPKNEVRRQPPYNVIILNDDDHTYRYFIEMFQKIFGFPPEKGFQYTEEMDR |
| | | TGRLILLTTSKEHAELKQDQLHSYGPDPYLGRPCSGSVTVVIEPAV |
| PS679 | 55 | MAFPARGKTAPKNEVRRQPPYNVIILNDDDHTYRYFLEMFQKIFGFPPEKGFQYAEEIDR |
| | | TGRLILLTTSKEHAELKQDQMHSYGPDPYLGRPCSGSITCVIEPAV |
| PS680 | 56 | MAFPARGKTAPKNEVRRQPPYNVIILNDDDHTYRYFIEMFQKIFGFPPEKGFQIVEEIDR |
| | | TGRYILLTTSKEHAELKQDQLHSYGPDPYLGRPCSGSITCVIEPAV |
| PS681 | 57 | MAFPARGKTAPKNEVRRQPPYNVIMLNDDDHTYRYFIELFQKIFGFPPEKGFQIIEEIDR |
| | | TGRAILLTTSKEHAELKQDQIHSYGPDPYLGRPCSGSITCVIEPAV |
| PS682 | 58 | MAFPARGKTAPKNEVRRQPPYNVIILNDDDHTYRYFLEMFQKIFGFPPEKGFQYVEEIDR |
| | | TGRIILLTTSKEHAELKQDQMHSYGPDPYLGRPCSGSITCVIEPAV |
| PS683 | 59 | MPTAASATESAIEDTPAPARPEVDGRTKPKRQPRYHVVIWNDDDHTYQYFVVMFQSLFGH |
| | | PPERGYRIVKEIDTQGRYIVLTTTREHAELKRDQLHAFGYDRLLARSKGSIKASIEAEE |
| PS684 | 60 | MPTAASATESAIEDTPAPARPEVDGRTKPKRQPRYHVVYWNDDDHTYQYFVVLFQSLFGH |
| | | PPERGYRIVKEIDTQGRYIVLTTTREHAELKRDQTHAFGYDRLLARSKGSIKISIEAEE |
| PS685 | 61 | MPTAASATESAIEDTPAPARPEVDGRTKPKRQPRYHVVMWNDDDHTYQYFVVLLQSLFGH |
| | | PPERGYRIVKEIDTQGRYIVLTTTREHAELKRDQIHAFGYDRLLARSKGSIKVSIEAEE |
| PS686 | 62 | MPTAASATESAIEDTPAPARPEVDGRTKPKRQPRYHVVFWNDDDHTYQYFVVLFQSLFGH |
| | | PPERGYRIAKEIDTQGRYIVLTTTREHAELKRDQVHAFGYDRLLARSKGSIKISIEAEE |
| PS687 | 63 | MPTAASATESAIEDTPAPARPEVDGRTKPKRQPRYHVVIWNDDDHTYQYFVVTFQSLFGH |
| | | PPERGYRIAKEIDTQGRYIVLTTTREHAELKRDQWHAFGYDRLLARSKGSIKCSIEAEE |
| PS688 | 64 | MHSKFSHAGRICGAKFKVGEPAYRCKECSFDDTCILCVNCFNPKDHTGHHVYTMICTEFL |
| | | NGICDCGDKEAWNHTLFCKAEEG |
| PS689 | 65 | MHSKFSHAGRICGAKFKVGEPAYLCKECSFDDTCILCVNCFNPKDHTGHHVYTMICTEFL |
| | | NGICDCGDKEAWNHTLFCKAEEG |
| PS710 | 66 | MSDSPVDLKPKPKVKPKLERPKLYKVMFLNDDYTPMSYIIVFFKAVFRMSEDTGRRKMMT |
| | | AHRFGSMVVVVCERDIAETKAKEFTDHGKEAGFPIMMTTEPEE |
| PS711 | 67 | MSDSPVDLKPKPKVKPKLERPKLYKVMFLDDDYTPMSYIIVFFKAVFRMSEDTGRRKMMT |
| | | AHRFGSMVVVVCERDIAETKAKEFTDHGKEAGFPIQMTTEPEE |
| PS712 | 68 | MIAEPICMQGEGDGEDGGTNRGTSVITRVKPKTKRPNLYRVLTLDDDYTPMEFMIHMFER |
| | | FFQKDREAATRLMLLVHQHGVAECGVFTYEVAETKVSQMMDWARQHQHPFQMVMEKK |
| PS713 | 69 | MPQERQQVTRKHYPNYKVILLDMDFMTFAFMSAVLMKYIPNMTSDRSTELIRQAHYEGQT |
| | | IVWVGPQEQAELYHEQFLRSGLQNMPLEPE |
| PS714 | 70 | MASAPSTTLDKSTQVVKKTYPNYKVIFLDSDLLTMDFLANVMIKYIPDMTTDRAWEKAYQ |
| | | MHYQGQFIVWTGPQEQAELYHQQFRREGLENIPLEAA |
| PS715 | 71 | MTSTLRARPARDTDLQHRPYPHYRIITLDNDVNTFQKIANVHVTFLPGMTRDQMWAKMQQ |
| | | VDGEGSVVVWTGPQEQAELYHVQFGNQGLKNIPLEPV |
| PS716 | 72 | MATETIERPRTRDPGSGLGGHWLVIMLDNDHMTFDLISKVLARVIPGVTVDDAYRFTYQM |
| | | HQRGQVIIWRGPKEPAEHYWEQLQDVGLDNAPLERH |
| PS717 | 73 | MAFPARGKTAPKNEVRRQPPYNVIILNSDDHTYRYFMEMFQKIFGFPPEKGFQYMEEIDR |
| | | TGRIILLTTSKEHAELKQDQSHSYGPDPYLGRPCSGSITMVIEPAV |
| PS718 | 74 | MGQTVEKPRVEGPGTGLGGSWRVISRDNDHYTFDEWVRIIARFIPGVSLERAHEWMKVLH |
| | | TTGRMVVYTGHKEAAEHYWQQLKGAGFQSVPLEQG |
| PS719 | 75 | MVSIGAATVACAEGRPIFSGYFDWLAAMPETVTVPRTRLRPKTERPKLYKVIFVDDDFVP |
| | | FEFIIRMFKAEFRMSEDQAAEKMMRAHQRGVQVVAVFTRDVAETKATRFTDWGRAKGYPL |
| | | IMTTEPEE |
| PS720 | 76 | MVSIGAATVACAEGRPIFSGYFDWLAAMPETVTVPRTRLRPKTERPKLHKVIFVDQDYIP |
| | | FEFIITMFKGEFHMSEDQAQRKLITAHRRGVYVVAVFTRDVAETKATRFSDAGRAKGYPL |
| | | QVTTEPEE |
| PS721 | 77 | MTLSVALGPDTQESTQTGTAVSTDTLTAPDIPWNLVFWDDPVTLMSRIIYFFQSYFGYSE |
| | | TKAYKIVMEAHKKGRSIVAHGSKEQVEQHAVAFHGLGLWTTVEKATGGNSGGGKSGGPGK |
| | | GKGKRG |
| PS722 | 78 | MSDTITLPGRPEVERDERTRRQPPYNVITHDKDDITFAYFIVMYNQLFGYPPEKGYEKLK |
| | | EIHLNGRAIVLTTSKEHAELKRDQMHAWGPDPFSSKDCKGSISASIEPAY |
| PS723 | 79 | MPTAASATESAIEDTPAPARPEVDGRTKPKRQPRYHVVIWNDDDWTYQYYVVMFQSLFGH |
| | | PPERGYRLMKELDTQGRFIVLTTTREHAELKRDQIHAFGYDRLLARSKGSIKASIEAEE |
| PS724 | 80 | MSSPSSLDDVQVSTSRAKPANETRTRKQPPYAVIFEDQDHVTHLWFYEMFMKVCGHAPEK |
| | | GFVKSQQIHTQGKVMVWSGTLELAELKRDQFRGFGPDNYAPAPVTFPPGMTIEPLP |
| PS725 | 81 | MSGTVVESKPRNSTQLAPRWKVIFHDNPVTTFAFIIGMFRRVFAKPPGEAREMLRRAHDT |
| | | GSVLVDVLALEQAEFRRDQFHSEARAEGFPSTMTLEPAD |
| PS726 | 82 | MHHHHHHHHHHDYDIPTTENLYFQGMHSKFSHAGRICGAKFKVGEPIYRCKECSFDDTCV |
| | | LCVNCFNPKDHTGHHVYTTICTEFNNGICDCGDKEAWNHTLFCKAEE |
| PS727 | 83 | MSDSPVDLKPKPKVKPKLERPKLYKVMFLNQDYVPMSFIVVMFKAVFRMSEDTGRKKMMH |
| | | AHRFGSVVVVVCERDIAETKAKEFTDYGKEAGFPVMMTTEPEE |
| PS728 | 84 | MTLSVALGPDTQESTQTGTAVSTDTLTAPDIPWNLVMWNQPVLLWSYMVYLFQSYFGYSE |
| | | TKTNKMVMEAHKKGRSIVAHGSKEQVEQHAVAMHGRGLWATVEKATGGNSGGGKSGGPGK |
| | | GKGKRG |
| PS729 | 85 | MSDTITLPGRPEVERDERTRRQPPYNVITHNQDDITWEYFRVMYNQLFGYPPEKGYEKLK |
| | | EIHLNGRIIVLTTSKEHAELKRDQMHAWGPDPFSSKDCKGSVSNSIEPAY |
| PS730 | 86 | MSDTITLPGRPEVERDERTRRQPPYNVIIHNTDDLTWEYFKVMFNQLFGYPPEKGYEKLK |
| | | EIHLNGRAIVLTTSKEHAELKRDQMHAWGPDPFSSKDCKGSVSASIEPAY |
| PS731 | 87 | MPTAASATESAIEDTPAPARPEVDGRTKPKRQPRYHVVTWNTDDWTHQYYVVMYQSLFGH |
| | | PPERGYRLTKEMDTQGRCIVLTTTREHAELKRDQMHAFGYDRLLARSKGSTKVSIEAEE |
| PS732 | 88 | MPTAASATESAIEDTPAPARPEVDGRTKPKRQPRYHVVMWNTDDWTYQYIIVMMQSLFGH |
| | | PPERGYRMVKEMDTQGRTIVLTTTREHAELKRDQMHAFGYDRLLARSKGSIKNSIEAEE |
| PS733 | 89 | MSSPSSLDDVQVSTSRAKPANETRTRKQPPYAVIFENQDHVTILWFWEMFMKVCGHAPEK |
| | | GFVKSQQIHTQGKVMVWSGTLELAELKRDQFRGFGPDNYAPRPVTFPPGMTIEPLP |
| PS734 | 90 | MSSPSSLDDVQVSTSRAKPANETRTRKQPPYAVIMENLDHITLLWMWEMFMKVCGHAPEK |
| | | GFVKSQQNHTQGKVMVWSGTLELAELKRDQMRGWGPDNYAPRPVTFPPGFTIEPLP |
| PS735 | 91 | MSGTVVESKPRNSTQLAPRWKVIYHDQPVTTFDFIIGMFRRVFAKPPGEAREMTRRAHDT |
| | | GSVLVDVLALEQAEFRRDQFHSEARAEGFPSTMTLEPAD |
| PS736 | 92 | MSGTVVESKPRNSTQLAPRWKVIYHDQPVMTFDFIIGLFRRVFAKPPGEARTITRIAHDT |
| | | GSVLVDVLALEQAEFRRDQFHSEARAEGFPATMTLEPAD |
| PS737 | 93 | MSDSPVDLKPKPKVKPKLERPKLYKVMFLNQDYTPMSFIVVMFKAVFRMSEDTGRKKMMH |
| | | AHRFGSVVVVVCERDIAETKAKEFTDYGKEAGFPSMMTTEPEE |
| PS738 | 94 | MIAEPICMQGEGDGEDGGTNRGTSVITRVKPKTKRPNLYRVLWLNHDYIPMEFMVHMFER |
| | | FFQKDREAATRYMLLVHQHGVAECGVFTYEVAETKVSQLMDWARQHQHPFQVVMEKK |
| PS739 | 95 | MIAEPICMQGEGDGEDGGTNRGTSVITRVKPKTKRPNLYRVLWLNHDYIPMEFMVHMFER |
| | | FFQKDREAATRIMLEVHQHGVSECGVFTYEVAETKVSQLMDFARQHQHPFQVVMEKK |
| PS740 | 96 | MPQERQQVTRKHYPNYKVIMLNNDFHTFQFMSAVMMKYIPNMTSDRSWEKVNQVHYEGQT |
| | | IVWVGPQEQAELYHEQFLRSGLTNMPLEPE |
| PS741 | 97 | MPQERQQVTRKHYPNYKVIMLNDDFWTFQFLAAVIMKYIPNMTSDRVWEITNQVHYEGQS |
| | | IVWVGPQEQAELYHEQFLREGFLHVPLEPE |
| PS742 | 98 | MASAPSTTLDKSTQVVKKTYPNYKVILLNNDLITRDKLANVLIKYIPDMTTDRAWERINQ |
| | | MHYQGQFIVWTGPQEQAELYHQQFRREGMQNIPLEAA |
| PS743 | 99 | MASAPSTTLDKSTQVVKKTYPNYKVIMLNNDLLTRDEIANVFIKYIPDMTTDRMWEMTNQ |
| | | MHYQGQLIVWTGPQEQAELYHQQFRREGLLNVPLEAA |
| PS744 | 100 | MTSTLRARPARDTDLQHRPYPHYRIITLDNDVITFQELVNYYVTFLPGMTRDQIWAKMQQ |
| | | VDGEGSAVVWTGPQEQAELYHVQLGNQGLFNCPLEPV |
| PS745 | 101 | MTSTLRARPARDTDLQHRPYPHYRIITLDMDVNTFQEIANYYVTFLPGMTRDQMWAWMQQ |
| | | VDGEGSVVVWTGPQEQAELYHVQLGNQGLYNIPLEPV |
| PS746 | 102 | MATETIERPRTRDPGSGLGGHWLVIHLNSDHFTFDEHAKWLARVIPGVTVDDAYRFTDQM |
| | | HQRGQMIVWRGPKEPAEHYWEQLQDVGLSQSPLERH |
| PS747 | 103 | MATETIERPRTRDPGSGLGGHWLVIMLNSDHFTFDEFSKWLARVIPGVTVDDAYRFTDQM |
| | | HQRGQVIVWRGPKEPAEHYWEQFQDIGLSQVPLERH |
| PS748 | 104 | MAFPARGKTAPKNEVRRQPPYNVIILNSDDHTYRYYMEMFQKIFGFPPEKGFQYMEEIDR |
| | | TGRIILLTTSKEHAELKQDQLHSYGPDPYLGRPCSGSITCVIEPAV |
| PS749 | 105 | MAFPARGKTAPKNEVRRQPPYNVIILNSDDHTYRYFMEMFQKIFGFPPEKGFQYMEEIDR |
| | | TGRIILLTTSKEHAELKQDQLHSYGPDPYLGRPCSGSITCVIEPAV |
| PS750 | 106 | MGQTVEKPRVEGPGTGLGGSWRVIMRNTDHITKDEFARSIARFIPGVSLERAHEKIKVMH |
| | | TTGRFVVYTGHKEAAEHYWQQFKGSGVQVMPLEQG |
| PS751 | 107 | MGQTVEKPRVEGPGTGLGGSWRVIMRNDDHHTKDKFARMIARFIPGVSLERAHEKIKVLH |
| | | TTGRMVVYTGHKEAAEHYWQQMKGAGVQNVPLEQG |
| PS752 | 108 | MVSIGAATVACAEGRPIFSGYFDWLAAMPETVTVPRTRLRPKTERPKLYKVIFVNRDFIP |
| | | MEFIIRMFKAEFRMSEDQAARKMMYAHQRGVYVVAVFTRDVAETKATRFTDWGRAKGYPL |
| | | LMTTEPEE |
| PS753 | 109 | MVSIGAATVACAEGRPIFSGYFDWLAAMPETVTVPRTRLRPKTERPKLYKVIFVNRDFIP |
| | | MEFIIRMFKAEFRMSEDQAATKMMLAHQRGVQVVAVFTRDVAETKATRFTDWGRAKGYPL |
| | | LMTTEPEE |
| PS754 | 110 | MVSIGAATVACAEGRPIFSGYFDWLAAMPETVTVPRTRLRPKTERPKLHKVIFVNQDYIP |
| | | WEFIVTLFKGEFHMSEDQAQRKMIIAHRRGVYVVAVFTRDVAETKATRTSDWGRAKGYPL |
| | | QFTTEPEE |
| PS755 | 111 | MVSIGAATVACAEGRPIFSGYFDWLAAMPETVTVPRTRLRPKTERPKLHKVIFVNKDYIP |
| | | WEFIVTMFKGEFHMSEDQAQRKMIIAHRRGVYVVAVFTRDVAETKATRFSDWGRAKGYPL |
| | | QMTTEPEE |
| PS756 | 112 | MVSIGAATVACAEGRPIFSGYFDWLAAMPETVTVPRTRLRPKTERPPLHKVILVNRDFIP |
| | | MEFIIRMLKAEFRTTGDEAQRKMIYAHMKGSYVVAVFTREIAESKATRFTEWARAEGFPM |
| | | LMTTEPEE |
| PS757 | 113 | MVSIGAATVACAEGRPIFSGYFDWLAAMPETVTVPRTRLRPKTERPPLHKVILVNQDFIP |
| | | WEFMIRFLKAEFRTTGDEAQKKMISAHMKGSHVVAVFTREIAESKATRMTEWARAEGFPL |
| | | LFTTEPEE |
| PS758 | 114 | MTLSVALGPDTQESTQTGTAVSTDTLTAPDIPWNLVIWNLPVLLWSFIVYLFQSYFGYSE |
| | | TKANKIVMEMHKKGRSIVAHGSKEQVEQHAVAFHGRGLWTTVEKATGGNSGGGKSGGPGK |
| | | GKGKRG |
| PS759 | 115 | MPTAASATESAIEDTPAPARPEVDGRTKPKRQPRYHVVLWNDDDHTHQYVVVMLQSLFGH |
| | | PPERGYRLAKEVDTQGRVIVLTTTREHAELKRDQIHAFGYDRLLARSKGSMKASIEAEE |
| PS760 | 116 | MPTAASATESAIEDTPAPARPEVDGRTKPKRQPRYHVVLWNDDDHTYQYVVMMLQSLFGH |
| | | PPERGYRLAKEVDTQGRVIVLTTTREHAELKRDQIHAFGYDRLLARSKGSMKASIEAEE |
| PS761 | 117 | MPTAASATESAIEDTPAPARPEVDGRTKPKRQPRYHVVLWNDDDHTHQYVVMMLQSLFGH |
| | | PPERGYRLAKEVDTQGRVIVLTTTREHAELKRDQIHAFGYDRLLARSKGSMKASIEAEE |
| PS762 | 118 | MPTAASATESAIEDTPAPARPEVDGRTKPKRQPRYHVVLWDDDDHTYQYVVVMLQSLFGH |
| | | PPERGYRLAKEVDTQGRVIVLTTTREHAELKRDQIHAFGYDRLLARSKGSMKASIEAEE |
| PS763 | 119 | MPTAASATESAIEDTPAPARPEVDGRTKPKRQPRYHVVLWDYDDHTYQYVVVMLQSLFGH |
| | | PPERGYRLAKEVDTQGRVIVLTTTREHAELKRDQIHAFGYDRLLARSKGSMKASIEAEE |
| PS764 | 120 | MPTAASATESAIEDTPAPARPEVDGRTKPKRQPRYHVVLWDYDDHTYQYVVVMLQSLFGH |
| | | PPERGYRLAKELDTQGRVIVLTTTREHAELKRDQIHAFGYDRLLARSKGSMKASIEAEE |
| PS765 | 121 | MPTAASATESAIEDTPAPARPEVDGRTKPKRQPRYHVVLWNDEDHTYQYVVVMLQSLFGH |
| | | PPERGYRLAKEVDTQGRVIVLTTTREHAELKRDQIHAFGYDRLLARSKGSMKASIEAEE |
| PS766 | 122 | MPTAASATESAIEDTPAPARPEVDGRTKPKRQPRYHVVLWNYDDHTYQYVVVMLQSLFGH |
| | | PPERGYRLAKEVDTQGRVIVLTTTREHAELKRDQIHAFGYDRLLARSKGSMKASIEAEE |
| PS767 | 123 | MTLSVALGPDTQESTQTGTAVSTDTLTAPDIPWNLVIWDDPVNLMSYVSYVFQSYFGYSE |
| | | TKANKLMMEVHKKGRSIVAHGSKEQVEQHAVAMHGYGLWATVEKATGGNSGGGKSGGPGK |
| | | GKGKRG |
| PS768 | 124 | MSDTITLPGRPEVERDERTRRQPPYNVILHDDDDHTFEYVIVMLNQLFGYPPEKGYEMAK |
| | | EVHLNGRVIVLTTSKEHAELKRDQIHAFGPDPFSSKDCKGSMSASIEPAY |
| PS769 | 125 | MPTAASATESAIEDTPAPARPEVDGRTKPKRQPRYHVVLWDDDDHTYQYVVVMLQSLFGH |
| | | PPERGYRLAKEVDTQGRVIVLTTTREHAELKRDQIHAFGYDRLLARSKGSMKASIEAEE |
| PS770 | 126 | MSSPSSLDDVQVSTSRAKPANETRTRKQPPYAVIVEDDDHHTFLYVIEALMKVCGHAPEK |
| | | GFVLAQQIHTQGKAMVWSGTLELAELKRDQLRGFGPDNYAPRPVTFPLGVTIEPLP |
| PS771 | 127 | MSGTVVESKPRNSTQLAPRWKVIVHDDPVTTFDFVLGVLRRVFAKPPGEARRITREAHDT |
| | | GSALVDVLALEQAEFRRDQAHSLARAEGFPLTLTLEPAD |
| PS772 | 128 | MSDSPVDLKPKPKVKPKLERPKLYKVMLLDDDYTPMSFVTVVLKAVFRMSEDTGRRVMMT |
| | | AHRFGSAVVVVCERDIAETKAKEATDLGKEAGFPLMFTTEPEE |
| PS773 | 129 | MIAEPICMQGEGDGEDGGTNRGTSVITRVKPKTKRPNLYRVLLLDDDYTPMEFVIHILER |
| | | FFQKDREAATRIMLHVHQHGVGECGVFTYEVAETKVSQVMDFARQHQHPLQCVMEKK |
| PS774 | 130 | MPQERQQVTRKHYPNYKVIVLDDDFNTFQHVAACLMKYIPNMTSDRAWELTNQVHYEGQA |
| | | IVWVGPQEQAELYHEQLLRAGLTMAPLEPE |
| PS775 | 131 | MASAPSTTLDKSTQVVKKTYPNYKVIVLDDDLNTFDHVANCLIKYIPDMTTDRAWELTNQ |
| | | VHYQGQAIVWTGPQEQAELYHQQLRREGLTMAPLEAA |
| PS776 | 132 | MATETIERPRTRDPGSGLGGHWLVIVLDDDHNTFDHVAKTLARVIPGVTVDDGYRFADQI |
| | | HQRGQAIVWRGPKEPAEHYWEQLQDAGLSMAPLERH |
| PS777 | 133 | MAFPARGKTAPKNEVRRQPPYNVILLDDDDHTYRYVIEMLQKIFGFPPEKGFQIAEEVDR |
| | | TGRVILLTTSKEHAELKQDQVHSYGPDPYLGRPCSGSMTCVIEPAV |
| PS778 | 134 | MGQTVEKPRVEGPGTGLGGSWRVIVRDDDHNTFDHVARTLARFIPGVSLERGHEIAKVIH |
| | | TTGRAVVYTGHKEAAEHYWQQLKGAGLTMAPLEQG |
| PS779 | 135 | MVSIGAATVACAEGRPIFSGYFDWLAAMPETVTVPRTRLRPKTERPKLYKVILVDDDFTP |
| | | REFVVRVLKAEFRMSEDQAAKVMMTAHQRGVCVVAVFTRDVAETKATRATDAGRAKGYPL |
| | | LFTTEPEE |
| PS780 | 136 | MVSIGAATVACAEGRPIFSGYFDWLAAMPETVTVPRTRLRPKTERPKLHKVILVDDDYTP |
| | | REFVVTVLKGEFHMSEDQAQRVMITAHRRGVCVVAVFTRDVAETKATRASDAGRAKGYPL |
| | | QFTTEPEE |
| PS781 | 137 | MVSIGAATVACAEGRPIFSGYFDWLAAMPETVTVPRTRLRPKTERPPLHKVILVDDDFTP |
| | | REFVVRLLKAEFRTTGDEAQRIMITAHMKGSCVVAVFTREIAESKATRATETARAEGFPL |
| | | LFTTEPEE |
| PS782 | 138 | MTLSVALGPDTQESTQTGTAVSTDTLTAPDIPWNLVIWDDPVNLMSYVSYVFQSYFGYSE |
| | | TKANKLMMEVDKKGRSIVAHGSKEQVEQHAVAMHGYGLWATVEKATGGNSGGGKSGGPGK |
| | | GKGKRG |
| PS783 | 139 | MSDTITLPGRPEVERDERTRRQPPYNVILHDDDDHTFEYVIVMLNQLFGYPPEKGYEMAK |
| | | EVDLNGRVIVLTTSKEHAELKRDQIHAFGPDPFSSKDCKGSMSASIEPAY |
| PS784 | 140 | MSSPSSLDDVQVSTSRAKPANETRTRKQPPYAVIVEDDDHHTFLYVIEALMKVCGHAPEK |
| | | GFVLAQQIDTQGKAMVWSGTLELAELKRDQLRGFGPDNYAPRPVTFPLGVTIEPLP |
| PS785 | 141 | MSGTVVESKPRNSTQLAPRWKVIVHDDPVTTFDFVLGVLRRVFAKPPGEARRITREADDT |
| | | GSALVDVLALEQAEFRRDQAHSLARAEGFPLTLTLEPAD |
| PS786 | 142 | MSDSPVDLKPKPKVKPKLERPKLYKVMLLDDDYTPMSFVTVVLKAVFRMSEDTGRRVMMT |
| | | ADRFGSAVVVVCERDIAETKAKEATDLGKEAGFPLMFTTEPEE |
| PS787 | 143 | MIAEPICMQGEGDGEDGGTNRGTSVITRVKPKTKRPNLYRVLLLDDDYTPMEFVIHILER |
| | | FFQKDREAATRIMLHVDQHGVGECGVFTYEVAETKVSQVMDFARQHQHPLQCVMEKK |
| PS788 | 144 | MPQERQQVTRKHYPNYKVIVLDDDFNTFQHVAACLMKYIPNMTSDRAWELTNQVDYEGQA |
| | | IVWVGPQEQAELYHEQLLRAGLTMAPLEPE |
| PS789 | 145 | MASAPSTTLDKSTQVVKKTYPNYKVIVLDDDLNTFDHVANCLIKYIPDMTTDRAWELTNQ |
| | | VDYQGQAIVWTGPQEQAELYHQQLRREGLTMAPLEAA |
| PS790 | 146 | MATETIERPRTRDPGSGLGGHWLVIVLDDDHNTFDHVAKTLARVIPGVTVDDGYRFADQI |
| | | DQRGQAIVWRGPKEPAEHYWEQLQDAGLSMAPLERH |
| PS791 | 147 | MGQTVEKPRVEGPGTGLGGSWRVIVRDDDHNTFDHVARTLARFIPGVSLERGHEIAKVID |
| | | TTGRAVVYTGHKEAAEHYWQQLKGAGLTMAPLEQG |
| PS792 | 148 | MVSIGAATVACAEGRPIFSGYFDWLAAMPETVTVPRTRLRPKTERPKLYKVILVDDDFTP |
| | | REFVVRVLKAEFRMSEDQAAKVMMTADQRGVCVVAVFTRDVAETKATRATDAGRAKGYPL |
| | | LFTTEPEE |
| PS793 | 149 | MVSIGAATVACAEGRPIFSGYFDWLAAMPETVTVPRTRLRPKTERPKLHKVILVDDDYTP |
| | | REFVVTVLKGEFHMSEDQAQRVMITADRRGVCVVAVFTRDVAETKATRASDAGRAKGYPL |
| | | QFTTEPEE |
| PS794 | 150 | MVSIGAATVACAEGRPIFSGYFDWLAAMPETVTVPRTRLRPKTERPPLHKVILVDDDFTP |
| | | REFVVRLLKAEFRTTGDEAQRIMITADMKGSCVVAVFTREIAESKATRATETARAEGFPL |
| | | LFTTEPEE |
| PS795 | 151 | MPTAASATESAIEDTPAPARPEVDGRTKPKRQPRYHVVLWDDDDHTYQYVVVMLRSLFGH |
| | | PPERGYRLAKEVDTQGRVIVLTTTREHAELKRDQIHAFGYDRLLARSKGSMKASIEAEE |
| PS796 | 152 | MPTAASATESAIEDTPAPARPEVDGRTKPKRQPRYHVVLWDDDDHTYQYVVVMLQSLFGH |
| | | PPERGYRLAKEMDTQGRVIVLTTTREHAELKRDQIHAFGYDRLLARSKGSMKASIEAEE |
| PS797 | 153 | MPTAASATESAIEDTPAPARPEVDGRTKPKRQPRYHVVLWDDDDHTYQYVVVMLQSLFGH |
| | | PPERGYRLAKEMDTLGRVIVLTTTREHAELKRDQIHAFGYDRLLARSKGSMKASIEAEE |
| PS798 | 154 | MPTAASATESAIEDTPAPARPEVDGRTKPKRQPRYHVVLWNDDDHTYQYVVVMLQSLFGH |
| | | PPERGYRLAKEMDTLGRVIVLTTTREHAELKRDQIHAFGYDRLLARSKGSMKASIEAEE |
| PS799 | 155 | MPTAASATESAIEDTPAPARPEVDGRTKPKRQPRYHVVLWNDDYHTYQYVVVMLQSLFGH |
| | | PPERGYRLAKEVDTQGRVIVLTTTREHAELKRDQIHAFGYDRLLARSKGSMKASIEAEE |
| PS800 | 156 | MPTAASATESAIEDTPAPARPEVDGRTKPKRQPRYHVVLWNDNYHTYQYVVVMLQSLFGH |
| | | PPERGYRLAKEVDTQGRVIVLTTTREHAELKRDQIHAFGYDRLLARSKGSMKASIEAEE |
| PS801 | 157 | MPTAASATESAIEDTPAPARPEVDGRTKPKRQPRYHVVLWNDDDHTYQYVVVMLQSLFGH |
| | | PPKRGYRLAKEVDTQGRVIVLTTTREHAELKRDQIHAFGYDRLLARSKGSMKASIEAEE |
| PS802 | 158 | MPTAASATESAIEDTPAPARPEVDGRTKPKRQPRYHVVLWNDDDHTYQYVVVMLQSLFGH |
| | | SPERGYRLAKEVDTQGRVIVLTTTREHAELKRDQIHAFGYDRLLARSKGSMKASIEAEE |
| PS803 | 159 | MPTAASATESAIEDTPAPARPEVDGRTKPKRQPRYHVVLWNDDDHTYQYVVVMLQSLFGH |
| | | PRERGYRLAKEVDTQGRVIVLTTTREHAELKRDQIHAFGYDRLLARSKGSMKASIEAEE |
| PS804 | 160 | MPTAASATESAIEDTPAPARPEVDGRTKPKRQPRYHVVLWNDADHTYQYVVVMLQSLFGH |
| | | PPERGYRLAKEVDTQGRVIVLTTTREHAELKRDQIHAFGYDRLLARSKGSMKASIEAEE |
| PS805 | 161 | MPTAASATESAIEDTPAPARPEVDGRTKPKRQPRYHVVLWNDDDHTYQYVVVMLQSLFGH |
| | | PPERGFRLAKEVDTQGRVIVLTTTREHAELKRDQIHAFGYDRLLARSKGSMKASIEAEE |
| PS806 | 162 | MPTAASATESAIEDTPAPARPEVDGRTKPKRQPRYHVVLWNDDDHTYQYVVVMLQSLFGH |
| | | PPERGYRLAKEVDTQGRVIVLTTTREHAELKRDQIHAFGYDRLLAHSKGSMKASIEAEE |
| PS807 | 163 | MPTAASATESAIEDTPAPARPEVDGRTKPKRQPRYHVVLWNDDDHTYQYVVVMLQSLFGH |
| | | PPERGYRLAKEVDTQGRVIVLTTTREHAELKRDQIHAFGYDRLLAGSKGSMKASIEAEE |
| PS808 | 164 | MPTAASATESAIEDTPAPARPEVDGRTKPKRQPRYHVVLWNDDDHTYQYVVVMLRSLFGH |
| | | PPERGYRLAKEVDTQGRVIVLTTTREHAELKRDQIHAFGYDRLLARSKGSMKASIEAEE |
| PS809 | 165 | MPTAASATESAIEDTPAPARPEVDGRTKPKRQPRYHVVLWNDDDHTYQYVVVMLQSLFGH |
| | | PPERGYRLAKEMDTQGRVIVLTTTREHAELKRDQIHAFGYDRLLARSKGSMKASIEAEE |
| PS810 | 166 | MPTAASATESAIEDTPAPARPEVDSRTKPKRQPRYHVVNWNDDDLTCQYLVVMLQSLFGH |
| | | PPERGYRLAKEVDTQGRVIVLTTTREHAELKRDQIHAFGYDRLLARSKGSIKASIEAEE |
| PS811 | 167 | MPTAASATESAIEDTPAPARPEVDSRTKPKRQPRYHVVNWNDDDLTCQYMVVMLQSLFGH |
| | | PPERGYRLAKEVDTQGRVIVLTTTREHAELKRDQIHAFGYDRLLARSKGSTKASIEAEE |
| PS812 | 168 | MPTAASATESAIEDTPAPARPEVDGRTKPKRQPRYHVVNWNDDDPTRQYMVVMLQSLFGH |
| | | PPERGYRLAKETDTQGRVIVLTTTREHAELKRDQIHAFGYDRLLARSKGSTKASIEAEE |
| PS813 | 169 | MPTAASATESAIEDTPAPARPEVDGRTKPKRQPRYHVVNWNDDDPTRQYLVVMLQSLFGH |
| | | PPERGYRLAKETDTQGRVIVLTTTREHAELKRDQIHAFGYDRLLARSKGSTKASIEAEE |
| PS814 | 170 | MPTAASATESAIEDTPAPARPEVDGRTKPKRQPRYHVVLWNDDDHTYQYVVVMLQSLFGH |
| | | PPKRGYRLAKEVDTQGRVIVLTTTREHAELKRDQIHAFGYDRLLARSKGSMKASIEAEE |
| PS815 | 171 | MPTAASATESAIEDTPAPARPEVDGRTKPKRQPRYHVVLWNDDDHTYQYVVVMLQSLFGH |
| | | PPERGYRLAKEMDTQGRVIVLTTTREHAELKRDQIHAFGYDRLLARSKGSMKASIEAEE |
| PS816 | 172 | MPTAASATESAIEDTPAPARPEVDGRTKPKRQPRYHVVLWDDDDHTYQYVVVMLQSLFGH |
| | | PPERGYRLAKEMDTQGRVIVLTTTREHAELKRDQIHAFGYDRLLARSKGSMKASIEAEE |
| PS817 | 173 | MPTAASATESAIEDTPAPARPEVDGRTKPKRQPRYHVVLWDDDDHTYQYVVVMLQSLFGH |
| | | PPKRGYRLAKEVDTQGRVIVLTTTREHAELKRDQIHAFGYDRLLARSKGSMKASIEAEE |
| PS818 | 174 | MPTAASATESAIEDTPAPARPEVDGRTKPKRQPRYHVVLWNDDDHTYQYVVVMLQSLFGH |
| | | PPKRGYRLAKEMDTQGRVIVLTTTREHAELKRDQIHAFGYDRLLARSKGSMKASIEAEE |
| PS819 | 175 | MPTAASATESAIEDTPAPARPEVDGRTKPKRQPRYHVVLWDDDDHTYQYVVVMLQSLFGH |
| | | PPKRGYRLAKEMDTQGRVIVLTTTREHAELKRDQIHAFGYDRLLARSKGSMKASIEAEE |
| PS820 | 176 | MPTAASGTESAIEDTPAPARPEVDGRTKPKRQPRYHVVLWDDDDHTYQYVVVMLRSLFGH |
| | | PPERGYRLAKEVDTQGRVIVLTTTREHAELKRDQIHAFGYDRLLARSKGSMKASIEAEE |
| PS821 | 177 | MPTAASGTESAIEDTPAPARPEVDGRTKPKRQPRYHVVLWDDDDHTYQYVVVMLRSLFGH |
| | | PPERGYRLAKEMDTQGRVIVLTTTREHAELKRDQIHAFGYDRLLARSKGSMKASIEAEE |
| PS822 | 178 | MPTAASGTESAIEDTPAPARPEVDGRTKPKRQPRYHVVLWDDDDHTYQYVVVMLRSLFGH |
| | | PPKRGYRLAKEMDTQGRVIVLTTTREHAELKRDQIHAFGYDRLLARSKGSMKASIEAEE |
| PS823 | 179 | MPTAASATESAFEDTPAPARPEVDGRTKPKRQPRYHVVLWDDDDHTYQYVVVMLRSLFGH |
| | | PPERGYRLAKEVDTQGRVIVLTTTREHAELKRDQIHAFGYDRLLARSKGSMKASIEAEE |
| PS824 | 180 | MPTAASATESAFEDTPAPARPEVDGRTKPKRQPRYHVVLWDDDDHTYQYVVVMLRSLFGH |
| | | PPERGYRLAKEMDTQGRVIVLTTTREHAELKRDQIHAFGYDRLLARSKGSMKASIEAEE |
| PS825 | 181 | MPTAASATESAFEDTPAPARPEVDGRTKPKRQPRYHVVLWDDDDHTYQYVVVMLRSLFGH |
| | | PPKRGYRLAKEVDTQGRVIVLTTTREHAELKRDQIHAFGYDRLLARSKGSMKASIEAEE |
| PS826 | 182 | MPTAASATESAFEDTPAPARPEVDGRTKPKRQPRYHVVLWDDDDHTYQYVVVMLRSLFGH |
| | | PPKRGYRLAKEMDTQGRVIVLTTTREHAELKRDQIHAFGYDRLLARSKGSMKASIEAEE |
| PS827 | 183 | MPTAASATESAIEDTPAPARPEVDGRTKPKRQPRYHVVLWNDDDHTYQYVVVMLQSLFGH |
| | | PRERGYRLAKEVDTLGRVIVLTTTREHAELKRDQIHAFGYDRLLARSKGSMKASIEAEE |
| PS828 | 184 | MPTAASATESAIEDTPAPARPEVDGRTKPKRQPRYHVVLWDDDDHTYQYVVVMLQSLFGH |
| | | PRERGYRLAKEVDTLGRVIVLTTTREHAELKRDQIHAFGYDRLLARSKGSMKASIEAEE |
| PS829 | 185 | MPTAASATESAIEDTPAPARPEVDGRTKPKRQPRYHVVLWDDDDHTYQYVVVMLQSLFGH |
| | | PRERGYRLAKEMDTLGRVIVLTTTREHAELKRDQIHAFGYDRLLARSKGSMKASIEAEE |
| PS830 | 186 | MPTAASATESAIEDTPAPARPEVDGRTKPKRQPRYHVVLWDDDDHTYQYVVVMLQSLFGH |
| | | PRKRGYRLAKEMDTLGRVIVLTTTREHAELKRDQIHAFGYDRLLARSKGSMKASIEAEE |
| PS831 | 187 | MPTAASATESAIEDTPAPARPEVDGRTKPKRQPRYHVVLWNDDDHTYQYVVVMLQSLFGH |
| | | PRKRGYRLAKEMDTLGRVIVLTTTREHAELKRDQIHAFGYDRLLARSKGSMKASIEAEE |
| PS832 | 188 | MPTAASATESAIEDTPAPARPVVDGRTKPKRQPRYHVVLWDDDDHTYQYVVVMLQSLFGH |
| | | PPERGYRLAKEVDTQGRVIVLTTTREHAELKRDQIHAFGYDRLLARSKGSMKASIEAEE |
| PS833 | 189 | MPTAASATESAIEDTPAPARPVVDGRTKPKRQPRYHVVLWDDDDHTYQYVVVMLQSLFGH |
| | | PPERGYRLAKEMDTQGRVIVLTTTREHAELKRDQIHAFGYDRLLARSKGSMKASIEAEE |
| PS834 | 190 | MPTAASATESAIEDTPAPARPVVDGRTKPKRQPRYHVVLWDDDDHTYQYVVVMLQSLFGH |
| | | PPKRGYRLAKEMDTQGRVIVLTTTREHAELKRDQIHAFGYDRLLARSKGSMKASIEAEE |
| PS835 | 191 | MPTAASATESAIEDTPAPARPVVDGRTKPKRQPRYHVVLWDDDDHTYQYVVVMLQSLFGH |
| | | PPKRGYRLAKEVDTQGRVIVLTTTREHAELKRDQIHAFGYDRLLARSKGSMKASIEAEE |
| PS836 | 192 | MPTAASATESAIEDTPAPARPEVDGRTKPKRQPRYHVVLWNDDDHTYQYVVVMLQSLFGH |
| | | PRERGYRLAKEVDTQGRVIVLTTTREHAELKRDQIHAFGYDRLLARSKGSMKASIEAEE |
| PS837 | 193 | MPTAASATESAIEDTPAPARPEVDGRTKPKRQPRYHVVLWDDDDHTYQYVVVMLQSLFGH |
| | | PRERGYRLAKEVDTQGRVIVLTTTREHAELKRDQIHAFGYDRLLARSKGSMKASIEAEE |
| PS838 | 194 | MPTAASATESAIEDTPAPARPEVDGRTKPKRQPRYHVVLWDDDDHTYQYVVVMLQSLFGH |
| | | PRERGYRLAKEMDTQGRVIVLTTTREHAELKRDQIHAFGYDRLLARSKGSMKASIEAEE |
| PS839 | 195 | MPTAASATESAIEDTPAPARPEVDGRTKPKRQPRYHVVLWDDDDHTYQYVVVMLQSLFGH |
| | | PRKRGYRLAKEMDTQGRVIVLTTTREHAELKRDQIHAFGYDRLLARSKGSMKASIEAEE |
| PS840 | 196 | MPTAASATESAIEDTPAPARPEVDGRTKPKRQPRYHVVLWNDDDHTYQYVVVMLQSLFGH |
| | | PRKRGYRLAKEVMDTQGRVIVLTTTREHAELKRDQIHAFGYDRLLARSKGSMKASIEAEE |
| PS841 | 197 | MPTAASATESAIEDTPAPARPEVDGRTKPKRQPRYHVVLWNDDDHTYQYVVVMLQSLFGH |
| | | SPKRGYRLAKEVDTQGRVIVLTTTREHAELKRDQIHAFGYDRLLARSKGSMKASIEAEE |
| PS842 | 198 | MPTAASATESAIEDTPAPARPEVDGRTKPKRQPRYHVVLWDDDDHTYQYVVVMLQSLFGH |
| | | SPKRGYRLAKEVDTQGRVIVLTTTREHAELKRDQIHAFGYDRLLARSKGSMKASIEAEE |
| PS843 | 199 | MPTAASATESAIEDTPAPARPEVDGRTKPKRQPRYHVVLWDDDDHTYQYVVVMLQSLFGH |
| | | SPKRGYRLAKEMDTQGRVIVLTTTREHAELKRDQIHAFGYDRLLARSKGSMKASIEAEE |
| PS844 | 200 | MPTAASATESAIEDTPAPARPEVDGRTKPKRQPRYHVVLWNDDDHTYQYVVVMLQSLFGH |
| | | SPKRGYRLAKEMDTQGRVIVLTTTREHAELKRDQIHAFGYDRLLARSKGSMKASIEAEE |
| PS845 | 201 | MPTAASATESAIEDTPAPARPEVDGRTKPKRQPRYHVVLWNDDDHTYQYVVVMLRSLFGH |
| | | PPERGYRLAKEVDTQGRVIVLTTTREHAELKRDQIHAFGYDRLLARSKGSMKASIEAEE |
| PS846 | 202 | MPTAASATESAIEDTPAPARPEVDGRTKPKRQPRYHVVLWDDDDHTYQYVVVMLRSLFGH |
| | | PPERGYRLAKEVDTQGRVIVLTTTREHAELKRDQIHAFGYDRLLARSKGSMKASIEAEE |
| PS847 | 203 | MPTAASATESAIEDTPAPARPEVDGRTKPKRQPRYHVVLWDDDDHTYQYVVVMLRSLFGH |
| | | PPERGYRLAKEMDTQGRVIVLTTTREHAELKRDQIHAFGYDRLLARSKGSMKASIEAEE |
| PS848 | 204 | MPTAASATESAIEDTPAPARPEVDGRTKPKRQPRYHVVLWDDDDHTYQYVVVMLRSLFGH |
| | | PPKRGYRLAKEMDTQGRVIVLTTTREHAELKRDQIHAFGYDRLLARSKGSMKASIEAEE |
| PS849 | 205 | MPTAASATESAIEDTPAPARPEVDGRTKPKRQPRYHVVLWNDDDHTYQYVVVMLRSLFGH |
| | | PPKRGYRLAKEMDTQGRVIVLTTTREHAELKRDQIHAFGYDRLLARSKGSMKASIEAEE |
| PS850 | 206 | MPTAASATESAIEDTPAPARPEVDGRTKPKHQPRYHVVLWNDDDHTYQYVVVMLRSLFGH |
| | | PPERGYRLAKEVDTQGRVIVLTTTREHAELKRDQIHAFGYDRLLARSKGSMKASIEAEE |
| PS851 | 207 | MPTAASATESAIEDTPAPARPEVDGRTKPKHQPRYHVVLWDDDDHTYQYVVVMLRSLFGH |
| | | PPERGYRLAKEVDTQGRVIVLTTTREHAELKRDQIHAFGYDRLLARSKGSMKASIEAEE |
| PS852 | 208 | MPTAASATESAIEDTPAPARPEVDGRTKPKHQPRYHVVLWDDDDHTYQYVVVMLRSLFGH |
| | | PPERGYRLAKEMDTQGRVIVLTTTREHAELKRDQIHAFGYDRLLARSKGSMKASIEAEE |
| PS853 | 209 | MPTAASATESAIEDTPAPARPEVDGRTKPKHQPRYHVVLWDDDDHTYQYVVVMLRSLFGH |
| | | PPKRGYRLAKEMDTQGRVIVLTTTREHAELKRDQIHAFGYDRLLARSKGSMKASIEAEE |
| PS854 | 210 | MPTAASATESAIEDTPAPARPEVDGRTKPKHQPRYHVVLWNDDDHTYQYVVVMLRSLFGH |
| | | PPKRGYRLAKEMDTQGRVIVLTTTREHAELKRDQIHAFGYDRLLARSKGSMKASIEAEE |
| PS855 | 211 | MPTAASATESAIEDTPAPARPEVDGRTKPKRQPRYHVVLWNDDDHTYQYVVVMLQSLFGH |
| | | PPERGYRMAKEVDTQGRVIVLTTTREHAELKRDQIHAFGYDRLLARSKGSMKASIEAEE |
| PS856 | 212 | MPTAASATESAIEDTPAPARPEVDGRTKPKRQPRYHVVLWDDDDHTYQYVVVMLQSLFGH |
| | | PPERGYRMAKEVDTQGRVIVLTTTREHAELKRDQIHAFGYDRLLARSKGSMKASIEAEE |
| PS857 | 213 | MPTAASATESAIEDTPAPARPEVDGRTKPKRQPRYHVVLWDDDDHTYQYVVVMLQSLFGH |
| | | PPERGYRMAKEMDTQGRVIVLTTTREHAELKRDQIHAFGYDRLLARSKGSMKASIEAEE |
| PS858 | 214 | MPTAASATESAIEDTPAPARPEVDGRTKPKRQPRYHVVLWDDDDHTYQYVVVMLQSLFGH |
| | | PPKRGYRMAKEMDTQGRVIVLTTTREHAELKRDQIHAFGYDRLLARSKGSMKASIEAEE |
| PS859 | 215 | MPTAASATESAIEDTPAPARPEVDGRTKPKRQPRYHVVLWNDDDHTYQYVVVMLQSLFGH |
| | | PPKRGYRMAKEMDTQGRVIVLTTTREHAELKRDQIHAFGYDRLLARSKGSMKASIEAEE |
| PS860 | 216 | MPTAASATESAIEDTPAPARPEVDGRTKPKRQPRYHVVLWNDDDHTYQYVVVMLQSLLGH |
| | | PPERGYRLAKEMDTQGRVIVLTTTREHAELKRDQIHAFGYDRLLARSKGSMKASIEAEE |
| PS861 | 217 | MPTAASATESAIEDTPAPARPEVDGRTKPKRQPRYHVVLWDDDDHTYQYVVVMLQSLLGH |
| | | PPERGYRLAKEMDTQGRVIVLTTTREHAELKRDQIHAFGYDRLLARSKGSMKASIEAEE |
| PS862 | 218 | MPTAASATESAIEDTPAPARPEVDGRTKPKRQPRYHVVLWDDDDHTYQYVVVMLQSLLGH |
| | | PPKRGYRLAKEMDTQGRVIVLTTTREHAELKRDQIHAFGYDRLLARSKGSMKASIEAEE |
| PS863 | 219 | MPTAASATESAIEDTPAPARPEVDGRTKPKRQPRYHVVLWNDDDHTYQYVVVMLQSLLGH |
| | | PPKRGYRLAKEMDTQGRVIVLTTTREHAELKRDQIHAFGYDRLLARSKGSMKASIEAEE |
| PS864 | 220 | MPTAASATESAIEDTPAPARPEVDGRTKPKRQPRYHVVLWNDDDHTYQYVVVMLQSLFGH |
| | | PPGRGYRLAKEVDTQGRVIVLTTTREHAELKRDQIHAFGYDRLLARSKGSMKASIEAEE |
| PS865 | 221 | MPTAASATESAIEDTPAPARPEVDGRTKPKRQPRYHVVLWDDDDHTYQYVVVMLQSLFGH |
| | | PPGRGYRLAKEVDTQGRVIVLTTTREHAELKRDQIHAFGYDRLLARSKGSMKASIEAEE |
| PS866 | 222 | MPTAASATESAIEDTPAPARPEVDGRTKPKRQPRYHVVLWDDDDHTYQYVVVMLQSLFGH |
| | | PPGRGYRLAKEMDTQGRVIVLTTTREHAELKRDQIHAFGYDRLLARSKGSMKASIEAEE |
| PS867 | 223 | MPTAASATESAIEDTPAPARPEVDGRTKPKRQPRYHVVLWNDDDHTYQYVVVMLQSLFGH |
| | | PPGRGYRLAKEMDTQGRVIVLTTTREHAELKRDQIHAFGYDRLLARSKGSMKASIEAEE |
| PS868 | 224 | MPTAASATESAIEDTPAPARPEVDGRTKPKRQPRYHVVLWNDDDHTYQYVVVMLQSLFGH |
| | | PPGRGYRLAKEVDTQGRVIVLTTTREHAELKRDQIHAFGYDRLLARSKGSMKASIEAEE |
| PS869 | 225 | MPTAASATESAIEDTPAPARPEVDGRTKPKRQPRYHVVLWDDDDHTYQYVVVMLQSLFGH |
| | | PPGRGYRLAKEVDTQGRVIVLTTTREHAELKRDQIHAFGYDRLLAHSKGSMKASIEAEE |
| PS870 | 226 | MPTAASATESAIEDTPAPARPEVDGRTKPKRQPRYHVVLWDDDDHTYQYVVVMLQSLFGH |
| | | PPGRGYRLAKEMDTQGRVIVLTTTREHAELKRDQIHAFGYDRLLAHSKGSMKASIEAEE |
| PS871 | 227 | MPSAAPAKPKTKRQSRTQGMPPYNVVLLDDDDHTYGYVIEMLRKVFGHPPEKGFELATEV |
| | | DKNGRVIVMTTNLEVAELKRDEVHAFGPDPLMPRSKGSMSAVVERAG |
| PS872 | 228 | MPSAAPAKPKTKRQSRTQGMPPYNVVLLDDDDHTYGYVIEMLNKVFGHPPEKGFELATEM |
| | | DKNGRVIVMTTNLEVAELKRDEVHAFGPDPLMPRSKGSMSAVVERAG |
| PS873 | 229 | MPSAAPAKPKTKRQSRTQGMPPYNVVLLDDDDHTYGYVIEMLRKVFGHPPEKGFELATEM |
| | | DKNGRVIVMTTNLEVAELKRDEVHAFGPDPLMPRSKGSMSAVVERAG |
| PS874 | 230 | MPSAAPAKPKTKRQSRTQGMPPYNVVLLDDDDHTYGYVIEMLNKVFGHPREKGFELATEV |
| | | DKLGRVIVMTTNLEVAELKRDEVHAFGPDPLMPRSKGSMSAVVERAG |
| PS875 | 231 | MPSAAPAKPKTKRQSRTQGMPPYNVVLLDDDDHTYGYVIEMLRKVFGHPREKGFELATEM |
| | | DKLGRVIVMTTNLEVAELKRDEVHAFGPDPLMPRSKGSMSAVVERAG |
| PS876 | 232 | MPSAAPAKPVTKRQSRTQGMPPYNVVLLDDDDHTYGYVIEMLNKVFGHPPEKGFELATEV |
| | | DKNGRVIVMTTNLEVAELKRDEVHAFGPDPLMPRSKGSMSAVVERAG |
| PS877 | 233 | MPSAAPAKPVTKRQSRTQGMPPYNVVLLDDDDHTYGYVIEMLRKVFGHPPEKGFELATEM |
| | | DKNGRVIVMTTNLEVAELKRDEVHAFGPDPLMPRSKGSMSAVVERAG |
| PS878 | 234 | MPSAAPAKPKTKRQSRTQGMPPYNVVLLDDDDHTYGYVIEMLNKVFGHPREKGFELATEV |
| | | DKNGRVIVMTTNLEVAELKRDEVHAFGPDPLMPRSKGSMSAVVERAG |
| PS879 | 235 | MPSAAPAKPKTKRQSRTQGMPPYNVVLLDDDDHTYGYVIEMLRKVFGHPREKGFELATEV |
| | | DKNGRVIVMTTNLEVAELKRDEVHAFGPDPLMPRSKGSMSAVVERAG |
| PS880 | 236 | MPSAAPAKPKTKRQSRTQGMPPYNVVLLDDDDHTYGYVIEMLRKVFGHPREKGFELATEM |
| | | DKNGRVIVMTTNLEVAELKRDEVHAFGPDPLMPRSKGSMSAVVERAG |
| PS881 | 237 | MPSAAPAKPKTKRQSRTQGMPPYNVVLLDDDDHTYGYVIEMLNKVFGHSPEKGFELATEV |
| | | DKNGRVIVMTTNLEVAELKRDEVHAFGPDPLMPRSKGSMSAVVERAG |
| PS882 | 238 | MPSAAPAKPKTKRQSRTQGMPPYNVVLLDDDDHTYGYVIEMLRKVFGHSPEKGFELATEM |
| | | DKNGRVIVMTTNLEVAELKRDEVHAFGPDPLMPRSKGSMSAVVERAG |
| PS883 | 239 | MPSAAPAKPKTKRQSRTQHMPPYNVVLLDDDDHTYGYVIEMLRKVFGHPPEKGFELATEV |
| | | DKNGRVIVMTTNLEVAELKRDEVHAFGPDPLMPRSKGSMSAVVERAG |
| PS884 | 240 | MPSAAPAKPKTKRQSRTQHMPPYNVVLLDDDDHTYGYVIEMLRKVFGHPPEKGFELATEM |
| | | DKNGRVIVMTTNLEVAELKRDEVHAFGPDPLMPRSKGSMSAVVERAG |
| PS885 | 241 | MPSAAPAKPKTKRQSRTQGMPPYNVVLLDDDDHTYGYVIEMLNKVFGHPPEKGFEMATEV |
| | | DKNGRVIVMTTNLEVAELKRDEVHAFGPDPLMPRSKGSMSAVVERAG |
| PS886 | 242 | MPSAAPAKPKTKRQSRTQGMPPYNVVLLDDDDHTYGYVIEMLRKVFGHPPEKGFEMATEM |
| | | DKNGRVIVMTTNLEVAELKRDEVHAFGPDPLMPRSKGSMSAVVERAG |
| PS887 | 243 | MPSAAPAKPKTKRQSRTQGMPPYNVVLLDDDDHTYGYVIEMLNKVLGHPPEKGFELATEM |
| | | DKNGRVIVMTTNLEVAELKRDEVHAFGPDPLMPRSKGSMSAVVERAG |
| PS888 | 244 | MPSAAPAKPKTKRQSRTQGMPPYNVVLLDDDDHTYGYVIEMLRKVLGHPPEKGFELATEM |
| | | DKNGRVIVMTTNLEVAELKRDEVHAFGPDPLMPRSKGSMSAVVERAG |
| PS889 | 245 | MPSAAPAKPKTKRQSRTQGMPPYNVVLLDDDDHTYGYVIEMLNKVFGHPPGKGFELATEV |
| | | DKNGRVIVMTTNLEVAELKRDEVHAFGPDPLMPRSKGSMSAVVERAG |
| PS890 | 246 | MPSAAPAKPKTKRQSRTQGMPPYNVVLLDDDDHTYGYVIEMLRKVFGHPPGKGFELATEM |
| | | DKNGRVIVMTTNLEVAELKRDEVHAFGPDPLMPRSKGSMSAVVERAG |
| PS891 | 247 | MPSAAPAKPKTKRQSRTQGMPPYNVVLLDDDDHTYGYVIEMLNKVFGHPPGKGFELATEV |
| | | DKNGRVIVMTTNLEVAELKRDEVHAFGPDPLMPHSKGSMSAVVERAG |
| PS892 | 248 | MPSAAPAKPKTKRQSRTQGMPPYNVVLLDDDDHTYGYVIEMLRKVFGHPPGKGFELATEM |
| | | DKNGRVIVMTTNLEVAELKRDEVHAFGPDPLMPHSKGSMSAVVERAG |
| PS896 | 249 | MPTAASATESAIEDTPAPARPEVDGRTKPKRQPRYHVVLWDDDDHTYQYVVVMLQSLFGH |
| | | PPKRGYRLAKEMDTLGRVIVLTTTREHAELKRDQIHAFGYDRLLARSKGSMKASIEAEE |
| PS897 | 250 | MPTAASATESAIEDTPAPARPEVDGRTKPKRQPRYHVVNWNDDDPTRQYTVVMLQSLFGH |
| | | PPERGYRLAKETRTQGRVIVLTTTREHAELKRDQIHAFGYDRLLARSKGSTKASIEAEE |
| PS898 | 251 | MPTAASATESAIEDTPAPARPEVDSRTKPKRQPRYHVVNWNDDDLTCQYTVVMLQSLFGH |
| | | PPERGYRLAKEVDTQGRVIVLTTTREHAELKRDQIHAFGYDRLLARSKGSHKASIEAEE |
| PS899 | 252 | MPTAASATESAIEDTPAPARPEVDGRTKPKRQPRYHVVSWNDDDHTSQYTVVMLQSLFGH |
| | | PPERGYRLAKELHTQGRVIVLTTTREHAELKRDQIHAFGYDRLLARSKGSPKASIEAEE |
| PS900 | 253 | MPTAASATESAIEDTPAPARPEVDGRTKPKRQPRYHVVLWNDDDHTYQYVVVMLQSLFGH |
| | | PPKRGYRMAKEVDTQGRVIVLTTTREHAELKRDQIHAFGYDRLLARSKGSMKASIEAEE |
| PS901 | 254 | MPTAASATESAIEDTPAPARPEVDGRTKPKRQPRYHVVLWNDDDHTYQYVVVMLQSLFGH |
| | | PRKRGYRLAKEVDTQGRVIVLTTTREHAELKRDQIHAFGYDRLLARSKGSMKASIEAEE |
| PS902 | 255 | MPTAASATESAIEDTPAPARPEVDGRTKPKRQPRYHVVLWNDDDHTYQYVVVMLQSLFGH |
| | | PPGRGYRLAKEVDTQGRVIVLTTTREHAELKRDQIHAFGYDRLLAHSKGSMKASIEAEE |
| PS903 | 256 | MPTAASATESAIEDTPAPARPEVDGRTKPKRQPRYHVVLWNDDDHTYQYVVVMLQSLFGH |
| | | PPKRGYRLAKEVDTQGRVIVLTTTREHAELKRDQIHAFGYDRLLAHSKGSMKASIEAEE |
| PS904 | 257 | MPTAASATESAIEDTPAPARPEVDGRTKPKRQPRYHVVLWNDDDHTYQYVVVMLQSLFGH |
| | | PPRRGYRLAKEVDTQGRVIVLTTTREHAELKRDQIHAFGYDRLLARSKGSMKASIEAEE |
| PS905 | 258 | MPTAASATESAIEDTPAPARPEVDGRTKPKRQPRYHVVLWNDDDHTYQYVVVMLQSLFGH |
| | | PPDRGYRLAKEVDTQGRVIVLTTTREHAELKRDQIHAFGYDRLLARSKGSMKASIEAEE |
| PS906 | 259 | MPTAASATESAIEDTPAPARPEVDGRTKPKRQPRYHVVLWDDDDHTYQYVVVMLQSLFGH |
| | | PPKRGYRLAKEVDTQGRVIVLTTTREHAELKRDQIHAFGYDRLLAHSKGSMKASIEAEE |
| PS907 | 260 | MPTAASATESAIEDTPAPARPEVDGRTKPKRQPRYHVVLWDDDDHTYQYVVVMLQSLFGH |
| | | PPKRGYRLAKEMDTQGRVIVLTTTREHAELKRDQIHAFGYDRLLAHSKGSMKASIEAEE |
| PS908 | 261 | MPTAASATESAIEDTPAPARPEVDGRTKPKRQPRYHVVLWNDDDHTYQYVVVMLRSLFGH |
| | | PPERGYRLAKEVDTQGRVIVLTTTREHAELKRDQIHAFGYDRLLAHSKGSMKASIEAEE |
| PS909 | 262 | MPTAASATESAIEDTPAPARPEVDGRTKPKRQPRYHVVLWNDNYHTYQYVVVMLQSLFGH |
| | | PPERGYRLAKEVDTQGRVIVLTTTREHAELKRDQIHAFGYDRLLAHSKGSMKASIEAEE |
| PS910 | 263 | MPTAASATESAIEDTPAPARPEVDGRTKPKRQPRYHVVLWNDNDHTYQYVVVMLQSLFGH |
| | | PPERGYRLAKEVDTQGRVIVLTTTREHAELKRDQIHAFGYDRLLAHSKGSMKASIEAEE |
| PS911 | 264 | MPTAASATESAIEDTPAPARPEVDGRTKPKRQPRYHVVLWDDNYHTYQYVVVMLQSLFGH |
| | | PPERGYRLAKEVDTQGRVIVLTTTREHAELKRDQIHAFGYDRLLAHSKGSMKASIEAEE |
| PS912 | 265 | MPTAASATESAIEDTPAPARPEVDGRTKPKRQPRYHVVLWDDDDHTYQYVVVMLQSLFGH |
| | | PPERGYRLAKEVDTQGRVIVLTTTREHAELKRDQIHAFGYDRLLAHSKGSMKASIEAEE |
| PS913 | 266 | MPSAAPAKPKTKRQSRTQGMPPYNVVLLDDDDHTYGYVIEMLNKVFGHPPKKGFELATEV |
| | | DKNGRVIVMTTNLEVAELKRDEVHAFGPDPLMPRSKGSMSAVVERAG |
| PS914 | 267 | MPSAAPAKPKTKRQSRTQGMPPYNVVLLDDDDHTYGYVIEMLNKVFGHPPEKGFELATEV |
| | | DKNGRVIVMTTNLEVAELKRDEVHAFGPDPLMPHSKGSMSAVVERAG |
| PS915 | 268 | MPSAAPAKPKTKRQSRTQGMPPYNVVLLDDDDHTYGYVIEMLNKVFGHPPKKGFELATEV |
| | | DKNGRVIVMTTNLEVAELKRDEVHAFGPDPLMPHSKGSMSAVVERAG |
| PS916 | 269 | MPSAAPAKPKTKRQSRTQGMPPYNVVLLDDDDHTYGYVIEMLNKVFGHPPEKGFELATEM |
| | | DKLGRVIVMTTNLEVAELKRDEVHAFGPDPLMPRSKGSMSAVVERAG |
| PS917 | 270 | MPSAAPAKPKTKRQSRTQGMPPYNVVLLDDNYHTYGYVIEMLNKVFGHPPEKGFELATEV |
| | | DKNGRVIVMTTNLEVAELKRDEVHAFGPDPLMPRSKGSMSAVVERAG |
| PS918 | 271 | MPTAASATESAIEDTPAPARPEVDGRTKPKRQPRYHVVLWNDDDHTYQYVVVMLQSLFGH |
| | | PPERGYRLAKEVDTQGRVIVLTTTREHAELKRDQIHAFGYDPLMPRSKGSMSAVVEAEE |
| PS919 | 272 | MPTAASATESAIEDTPAPARPEVDGRTKPKRQPRYHVVLWNDDDHTYQYVVVMLQSLFGH |
| | | PPERGYRLAKEVDTQGRVIVLTTTREHAELKRDQIHAFGYDPLMPRSKGSMSASIEAEE |
| PS920 | 273 | MPTAASATESAIEDTPAPARPEVDGRTKPKRQPRYHVVLWNDDDHTYQYVVVMLQSLFGH |
| | | PPERGYRLAKEVDTQGRVIVLTTTREHAELKRDQIHAFGPDPLMPRSKGSMSAVVERAG |
| PS921 | 274 | MPTAASATESAFEDTPAPARPVVDGRTKPKHQPRYHVVLWDDDDHTYQYVVVMLRSLFGH |
| | | PRERGYRMAKEMDTLGRVIVLTTTREHAELKRDQIHAFGYDRLLARSKGSMKASIEAEE |
| PS922 | 275 | MPTAASATESAFEDTPAPARPVVDGRTKPKRQPRYHVVLWDDDDHTYQYVVVMLRSLFGH |
| | | PRERGYRMAKEMDTLGRVIVLTTTREHAELKRDQIHAFGYDRLLARSKGSMKASIEAEE |
| PS923 | 276 | MPTAASATESAFEDTPAPARPEVDGRTKPKRQPRYHVVLWDDDDHTYQYVVVMLRSLFGH |
| | | PRERGYRMAKEMDTLGRVIVLTTTREHAELKRDQIHAFGYDRLLARSKGSMKASIEAEE |
| PS924 | 277 | MPTAASATESAFEDTPAPARPEVDGRTKPKRQPRYHVVLWDDDDHTYQYVVVMLRSLFGH |
| | | PRERGYRMAKEMDTQGRVIVLTTTREHAELKRDQIHAFGYDRLLARSKGSMKASIEAEE |
| PS925 | 278 | MPTAASATESAIEDTPAPARPVVDGRTKPKHQPRYHVVLWDDDDHTYQYVVVMLRSLFGH |
| | | PRERGYRMAKEMDTLGRVIVLTTTREHAELKRDQIHAFGYDRLLARSKGSMKASIEAEE |
| PS926 | 279 | MPTAASATESAIEDTPAPARPVVDGRTKPKHQPRYHVVLWDDDDHTYQYVVVMLQSLFGH |
| | | PRERGYRMAKEMDTLGRVIVLTTTREHAELKRDQIHAFGYDRLLARSKGSMKASIEAEE |
| PS927 | 280 | MPTAASATESAIEDTPAPARPVVDGRTKPKHQPRYHVVLWDDDDHTYQYVVVMLQSLFGH |
| | | PPERGYRMAKEMDTLGRVIVLTTTREHAELKRDQIHAFGYDRLLARSKGSMKASIEAEE |
| PS928 | 281 | MPTAASATESAFEDTPAPARPEVDGRTKPKRQPRYHVVLWDDDDHTYQYVVVMLRSLFGH |
| | | PPERGYRMAKEMDTQGRVIVLTTTREHAELKRDQIHAFGYDRLLARSKGSMKASIEAEE |
| PS929 | 282 | MPTAASATESAIEDTPAPARPEVDGRTKPKRQPRYHVVLWDDDDHTYQYVVVMLQSLFGH |
| | | PRERGYRMAKEMDTLGRVIVLTTTREHAELKRDQIHAFGYDRLLARSKGSMKASIEAEE |
| PS930 | 283 | MPTAASATESAIEDTPAPARPEVDGRTKPKHQPRYHVVLWDDDDHTYQYVVVMLRSLFGH |
| | | PPERGYRMAKEMDTQGRVIVLTTTREHAELKRDQIHAFGYDRLLARSKGSMKASIEAEE |
| PS931 | 284 | MPTAASATESAIEDTPAPARPVVDGRTKPKHQPRYHVVLWDDDDHTYQYVVVMLQSLFGH |
| | | PPERGYRMAKEMDTQGRVIVLTTTREHAELKRDQIHAFGYDRLLARSKGSMKASIEAEE |
| PS932 | 285 | MPTAASATESAIEDTPAPARPVVDGRTKPKRQPRYHVVLWDDDDHTYQYVVVMLQSLFGH |
| | | PPERGYRMAKEMDTQGRVIVLTTTREHAELKRDQIHAFGYDRLLARSKGSMKASIEAEE |
| PS933 | 286 | MPTAASATESAIEDTPAPARPEVDGRTKPKRQPRYHVVLWDDDDHTYQYVVVMLRSLFGH |
| | | PPERGYRMAKEMDTQGRVIVLTTTREHAELKRDQIHAFGYDRLLARSKGSMKASIEAEE |
| PS934 | 287 | MPTAASATESAIEDTPAPARPEVDGRTKPKRQPRYHVVLWDDDDHTYQYVVVMLQSLFGH |
| | | PRERGYRMAKEMDTQGRVIVLTTTREHAELKRDQIHAFGYDRLLARSKGSMKASIEAEE |
| PS935 | 288 | MPTAASATESAIEDTPAPARPEVDGRTKPKRQPRYHVVLWDDDDHTYQYVVVMLQSLFGH |
| | | PPERGYRMAKEMDTLGRVIVLTTTREHAELKRDQIHAFGYDRLLARSKGSMKASIEAEE |
| PS936 | 289 | MPTAASATESAIEDTPAPARPEVDGRTKPKRQPRYHVVLWDDDDHTYQYVVVMLRSLFGH |
| | | PPERGYRMAKEMDTQGRVIVLTTTREHAELKRDQIHAFGYDRLLARSKGSMKASIEAEE |
| PS937 | 290 | MPTAASATESAFEDTPAPARPEVDGRTKPKRQPRYHVVLWDDDDHTYQYVVVMLQSLFGH |
| | | PPERGYRMAKEMDTQGRVIVLTTTREHAELKRDQIHAFGYDRLLARSKGSMKASIEAEE |
| PS938 | 291 | MPTAASATESAIEDTPAPARPEVDGRTKPKHQPRYHVVLWDDDDHTYQYVVVMLQSLFGH |
| | | PPERGYRMAKEMDTQGRVIVLTTTREHAELKRDQIHAFGYDRLLARSKGSMKASIEAEE |
| PS939 | 292 | MPTAASATESAFEDTPAPARPVVDGRTKPKHQPRYHVVLWDDDDHTYQYVVVMLRSLFGH |
| | | PRERGYRLAKEMDTLGRVIVLTTTREHAELKRDQIHAFGYDRLLARSKGSMKASIEAEE |
| PS940 | 293 | MPTAASATESAFEDTPAPARPVVDGRTKPKRQPRYHVVLWDDDDHTYQYVVVMLRSLFGH |
| | | PRERGYRLAKEMDTLGRVIVLTTTREHAELKRDQIHAFGYDRLLARSKGSMKASIEAEE |
| PS941 | 294 | MPTAASATESAFEDTPAPARPEVDGRTKPKRQPRYHVVLWDDDDHTYQYVVVMLRSLFGH |
| | | PRERGYRLAKEMDTLGRVIVLTTTREHAELKRDQIHAFGYDRLLARSKGSMKASIEAEE |
| PS942 | 295 | MPTAASATESAFEDTPAPARPEVDGRTKPKRQPRYHVVLWDDDDHTYQYVVVMLRSLFGH |
| | | PRERGYRLAKEMDTQGRVIVLTTTREHAELKRDQIHAFGYDRLLARSKGSMKASIEAEE |
| PS943 | 296 | MPTAASATESAIEDTPAPARPVVDGRTKPKHQPRYHVVLWDDDDHTYQYVVVMLRSLFGH |
| | | PRERGYRLAKEMDTLGRVIVLTTTREHAELKRDQIHAFGYDRLLARSKGSMKASIEAEE |
| PS944 | 297 | MPTAASATESAIEDTPAPARPVVDGRTKPKHQPRYHVVLWDDDDHTYQYVVVMLQSLFGH |
| | | PRERGYRLAKEMDTLGRVIVLTTTREHAELKRDQIHAFGYDRLLARSKGSMKASIEAEE |
| PS945 | 298 | MPTAASATESAIEDTPAPARPVVDGRTKPKHQPRYHVVLWDDDDHTYQYVVVMLQSLFGH |
| | | PPERGYRLAKEMDTLGRVIVLTTTREHAELKRDQIHAFGYDRLLARSKGSMKASIEAEE |
| PS946 | 299 | MPTAASATESAIEDTPAPARPVVDGRTKPKHQPRYHVVLWDDDDHTYQYVVVMLQSLFGH |
| | | PPERGYRLAKEMDTQGRVIVLTTTREHAELKRDQIHAFGYDRLLARSKGSMKASIEAEE |
| PS947 | 300 | MPTAASATESAFEDTPAPARPEVDGRTKPKRQPRYHVVLWDDDDHTYQYVVVMLQSLFGH |
| | | PPERGYRLAKEMDTQGRVIVLTTTREHAELKRDQIHAFGYDRLLARSKGSMKASIEAEE |
| PS948 | 301 | MPTAASATESAIEDTPAPARPEVDGRTKPKHQPRYHVVLWDDDDHTYQYVVVMLQSLFGH |
| | | PPERGYRLAKEMDTQGRVIVLTTTREHAELKRDQIHAFGYDRLLARSKGSMKASIEAEE |
| PS949 | 302 | MPTAASATESAFEDTPAPARPVVDGRTKPKHQPRYHVVLWDDNYHTYQYVVVMLRSLFGH |
| | | PRERGYRMAKEMDTLGRVIVLTTTREHAELKRDQIHAFGYDRLLAHSKGSMKASIEAEE |
| PS950 | 303 | MPTAASATESAIEDTPAPARPEVDGRTKPKRQPRYHVVLWDDNYHTYQYVVVMLQSLFGH |
| | | PPERGYRMAKEMDTQGRVIVLTTTREHAELKRDQIHAFGYDRLLAHSKGSMKASIEAEE |
| PS951 | 304 | MPTAASATESAIEDTPAPARPEVDGRTKPKRQPRYHVVLWDDDYHTYQYVVVMLQSLFGH |
| | | PPERGYRMAKEMDTQGRVIVLTTTREHAELKRDQIHAFGYDRLLAHSKGSMKASIEAEE |
| PS952 | 305 | MPTAASATESAIEDTPAPARPEVDGRTKPKRQPRYHVVLWDDDDHTYQYVVVMLQSLFGH |
| | | PPERGYRMAKEMDTQGRVIVLTTTREHAELKRDQIHAFGYDRLLAHSKGSMKASIEAEE |
| PS953 | 306 | MPTAASATESAIEDTPAPARPEVDGRTKPKRQPRYHVVLWDDDYHTYQYVVVMLQSLFGH |
| | | PPERGYRMAKEMDTQGRVIVLTTTREHAELKRDQIHAFGYDRLLARSKGSMKASIEAEE |
| PS954 | 307 | MPTAASATESAIEDTPAPARPEVDGRTKPKRQPRYHVVLWDDNDHTYQYVVVMLQSLFGH |
| | | PPERGYRMAKEMDTQGRVIVLTTTREHAELKRDQIHAFGYDRLLARSKGSMKASIEAEE |
| PS955 | 308 | MPTAASATESAFEDTPAPARPVVDGRTKPKHQPRYHVVLWDDDDHTYQYVVVMLRSLFGH |
| | | PRERGYRLAKEMDTLGRVIVLTTTREHAELKRDQIHAFGYDRLLARSKGSMKASIEAEE |
| PS956 | 309 | MPTAASATESAIEDTPAPARPEVDGRTKPKHQPRYHVVLWDDDDHTYQYVVVMLRSLFGH |
| | | PPERGYRLAKEMDTLGRVIVLTTTREHAELKRDQIHAFGYDRLLARSKGSMKASIEAEE |
| PS957 | 310 | MPTAASATESAIEDTPAPARPEVDGRTKPKHQPRYHVVLWDDDDHTYQYVVVMLRSLFGH |
| | | PRERGYRLAKEMDTQGRVIVLTTTREHAELKRDQIHAFGYDRLLARSKGSMKASIEAEE |
| PS958 | 311 | MPTAASATESAIEDTPAPARPVVDGRTKPKHQPRYHVVLWDDDDHTYQYVVVMLRSLFGH |
| | | PPERGYRLAKEMDTQGRVIVLTTTREHAELKRDQIHAFGYDRLLARSKGSMKASIEAEE |
| PS959 | 312 | MHHHHHHHHHHDYDIPTTENLYFQGMPTAASATESAIEDTPAPARPEVDGRTKPKHQPRY |
| | | HVVLWDDDDHTYQYVVVMLRSLFGHPPERGYRLAKEMDTQGRVIVLTTTREHAELKRDQI |
| | | HAFGYDRLLARSKGSMKASIEAEE |
| PS960 | 313 | MPTAASATESAIEDTPAPARPEVDGRTKPKRQPRYHVVLWDDDDHTYQYVVVMLRSLFGI |
| | | PPERGYRLAKEMDTQGRVIVLTTTREHAELKRDQIHAFGYDRLLARSKGSMKASIEAEE |
| PS961 | 314 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAFGRDRLLARSKGSMKASIEAEE |
| PS962 | 315 | MPTAASATESAIEDTPAPARPEVDGRTKPKRQPRYHVVLWDDDDHTYQYVVVMLRSLFGH |
| | | PYARGYRLAKEMDTQGRVIVLTTTREHAELKRDQIHAFGRDRLLARSKGSMKASIEAEE |
| PS963 | 316 | MPTAASATESAIEDTPAPARPEVDGRTKPKRQPRYHVVLWDDDDHTYQYVVVMLRSLFGH |
| | | PPARGYRLAKEMDTQGRVIVLTTTREHAELKRDQIHAFGRDRLLARSKGSMKASIEAEE |
| PS964 | 317 | MPTAASATESAIEDTPAPARPEVDGRTKPKRQPRYHVVLWDDDDHTYQYVVVMLRSLFGH |
| | | PPWRGYRLAKEMDTQGRVIVLTTTREHAELKRDQIHAFGYDRLLAHSKGSMKASIEAEE |
| PS965 | 318 | MPTAASATESAIEDTPAPARPEVDGRTKPKRQPRYHVVLWDDDDHTYQYVVVMLQSLFGH |
| | | PPSRGYRMAKEMDTQGRVIVLTTTREHAELKRDQIHAFGYDRLLAHSKGSMKASIEAEE |
| PS966 | 319 | MPTAASATESAIEDTPAPARPEVDGRTKPKRQPRYHVVLWDDDDHTYQYVVVMLQSLFGH |
| | | PPARGYRLAKEMDTQGRVIVLTTTREHAELKRDQIHAFGYDRLLAHSKGSMKASIEAEE |
| PS967 | 320 | MPTAASATESAIEDTPAPARPEVDGRTKPKRQPRYHVVLWDDDDHTYQYVVVMLRSLFGH |
| | | PPARGYRLAKEMDTQGRVIVLTTTREHAELKRDQIHAFGRDRLLARSKGSMKASIEAEE |
| PS968 | 32 | MPTAASATESAIEDTPAPARPEVDGRTKPKRQPRYHVVLWNDDDHTYQYVVVMLRSLFGH |
| | | PPERGYRLAKEMDTQGRVIVLTTTREHAELKRDQIHAFGRDRLLARSKGSMKASIEAEE |
| PS969 | 322 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAFGRDRLLAHSKGSMKASIEAEE |
| PS970 | 323 | MPTAASATESAIEDTPAPARPEVDGRTKPKRQPRYHVVLWNDDDHTYQYVVVMLRSLFGH |
| | | PPSRGYRLAKEMDTQGRVIVLTTTREHAELKRDQIHAFGYDRLLARSKGSMKASIEAEE |
| PS971 | 324 | MPTAASATESAIEDTPAPARPEVDGRTKPKHQPRYHVVLWDDDDHTYQYVVVMLRSLFGH |
| | | PPERGYRLAKEMDTQGRVIVLTTTREHAELKRDQIHAFGYDRLLAHSKGSMKASIEAEE |
| PS972 | 325 | MPTAASATESAIEDTPAPARPEVDGRTKPKHQPRYHVVLWDDDDHTYQYVVVMLHSLFGH |
| | | PPERGYRLAKEMDTQGRVIVLTTTREHAELKRDQIHAFGYDRLLAHSKGSMKASIEAEE |
| PS973 | 326 | MPTAASATESAIEDTPAPARPEVDGRTKPKHQPRYHVVLWDDDDHTYQYLVVMLRSLFGH |
| | | PPERGYRLAKEMDTQGRVIVLTTTREHAELKRDQIHAFGYDRLLARSKGSMKASIEAEE |
| PS974 | 327 | MPTAASATESAIEDTPAPARPEVDGRTKPKHQPRYHVVLWDDDDHTYQYIVVMLRSLFGH |
| | | PPERGYRLAKEMDTQGRVIVLTTTREHAELKRDQIHAFGYDRLLARSKGSMKASIEAEE |
| PS975 | 328 | MPTAASATESAIEDTPAPARPEVDGRTKPKHQPRYHVVLWDDDDHTYQYVVVMLRSLFGH |
| | | PPERGYRLAKEFDTQGRVIVLTTTREHAELKRDQIHAFGYDRLLARSKGSMKASIEAEE |
| PS976 | 329 | MPTAASATESAIEDTPAPARPEVDGRTKPKHQPRYHVVLWDDNYHTYQYVVVMLRSLFGH |
| | | PPERGYRLAKEMDTQGRVIVLTTTREHAELKRDQIHAFGYDRLLARSKGSMKASIEAEE |
| PS978 | 330 | MPTAASATESAIEDTPAPARPEVDGRTKPKRQPRYHVVLWDDDDHTYQYVVVMLHSLFGH |
| | | PPERGYRLAKEMDTQGRVIVLTTTREHAELKRDQIHAFGYDRLLARSKGSMKASIEAEE |
| PS979 | 331 | MPTAASGTESAIEDTPAPARPEVDGRTKPKRQPRYHVVLWDDDDHTYQYVVEMLRSLFGH |
| | | PPERGYRLAKEMDTQGRVIVLTTTREHAELKRDQIHAFGYDRLLARSKGSMKASIEAEE |
| PS980 | 332 | MPTAASATESAFEDTPAPARPEVDGRTKPKRQPRYHVVLWDDDDHTYQYVVVMLRSLFGH |
| | | PTERGYRLAKEMDTQGRVIVLTTTREHAELKRDQIHAFGYDRLLARSKGSMKASIEAEE |
| PS981 | 333 | MPTAASATESAIEDTPAPARPEVDGRTKPKRQPRYHVVLWDDDDHTYQYVVVMLRSLFGH |
| | | PPDRGYRLAKEMDTQGRVIVLTTTREHAELKRDQIHAFGYDRLLARSKGSMKASIEAEE |
| PS982 | 334 | MPTAASATESAIEDTPAPARPEVDGRTKPKRQPRYHVVLWDDDDHTYQYVVVMLQSLLGH |
| | | PPERGYRLAKEMDTQGRVIVLTTTREHAELKRDQIHAFGYDRLLARSKGSMKASIEAEE |
| PS983 | 335 | MPTAASATESAIEDTPAPARPEMDGRTKPKRQPRYHVVLWDDDDHTYQYVVVMLRSLFGH |
| | | PPERGYRLAKEMDTQGRVIVLTTTREHAELKRGQIHAFGYDRLLARSKGSMKASIEAEE |
| PS984 | 336 | MPTAASATESAIEDTPAPARPEVDGRTKPKRQPRYYVVLWDDDDHTYQYVVVMLRSLFGH |
| | | PPERGYRLAKEMDTQGRVIVLTTTREHAELKRDQIHAFGYDRLLARSKGSMKASIEAEE |
| PS985 | 337 | MPTAASATESAIEDTPAPARPEVDGRTKPKRQTRYHVVLWDDDDHTYQYVVVMLQSLFGH |
| | | PPERGYRLAKEMDTQGRVIVLTTTREHAELKRDQIHAFGYDRLLARSKGSMKASIEAEE |
| PS986 | 338 | MPTAASATESAIEDTPAPARPEVDGRTVPKRQPRYHVVLWDDDDHTYQYVVVMLQSLFGH |
| | | PPERGYHLAKEMDTQGRVIVLTTTREHAELKRDQIHAFGYDRLLARSKGSMKASIEAEE |
| PS987 | 339 | MPTAASATESAIEDTPAPARPEVDGRTKPKRQPRYHVVLWDDDDHTYQYVVVMLQSLFGH |
| | | PPERGYRLAKEMDTQGRVVVLTTTREHAELKRDQIHAFGYDRLLARSKGSMKASIEAEE |
| PS988 | 340 | MPTAASATESAIEDTPAPARPEVDGRTVPKRQPRYHVVLWDDDDHTYQYVVVMLQSLFGH |
| | | PPERGYRMAKEMDTQGRVIVLTTTREHAELKRDQIHAFGYDRLLARSKGSMKASIEAEE |
| PS989 | 341 | MPTAASATESAIEDTPAPARPEVDGRTKPRRQPRYHVVLWDDDDHTYQYVVVMLRSLFGH |
| | | PPERGYRLAKEMDTQGRVIVLTTTREHAELKRDQIHAFGYDRLLARSKGSMKASIEAEE |
| PS990 | 342 | MPTAASATESAIEDTPAPARPEVDGRTVPKRQPRYHVVLWDDDDHTYQYVVVMLQSLFGH |
| | | PPERGYRLAKEMDTQGRVIVLTTTREHAELKRDQIHAFGYDRLLARSKGSMKASIEAEE |
| PS991 | 343 | MPTAASATESAFEDTPAPARPEVDGRTKPIRQPRYHVVLWDDDDHTYQYVVVMLRSLFGH |
| | | PPERGYRLAKEMDTQGRVIVLTTTREHAELKRDQIHAFGYDRLLARSKGSMKASIEAEE |
| PS992 | 344 | MPTAASATESAIEDTPAPARPEVDGRTKPKRQPRCHVVLWDDDDHTYQYVVVMLRSLFGH |
| | | PPERGYRLAKEMDTQGRVIVLTTTREHAELKRDQIHAFGYDRLLARSKGSMKASIEAEE |
| PS993 | 345 | MPTAASATESAIEDTPAPARSEVDGRTKPKRQPRYHVVLWDDDDHTYQYVVVMLQSLFGH |
| | | PPERGYRLAKEMDTQGRVIVLTTTREHAELKRDQIHAFGYDRLLARSKGSMKASIEAEE |
| PS994 | 346 | MPTAASATESAIEDTPAPARPEVDGRTKPERQPRYHVVLWDDDDHTYQYVVVMLQSLFGH |
| | | PPERGYRLAKEMDTQGRVIVLTTTREHAELKRDQIHAFGYDRLLARSKGSMKASIEAEE |
| PS995 | 347 | MPTAASATESAIEDTPAPARPEVDGCTKPKRQPRYHVVLWDDDDHTYQYVVVMLQSLFGH |
| | | PPERGYRLAKEMDTQGRVIVLTTTREHAELKRDQIHAFGYDRLLARSKGSMKASIEAEE |
| PS996 | 348 | MPTAASATESAIEDTPAPARPEVDGSTKPKRQPRYHVVLWDDDDHTYQYVVVMLQSLFGH |
| | | PPERGYRLAKEMDTQGRVIVLTTTREHAELKRDQIHAFGYDRLLARSKGSMKASIEAEE |
| PS997 | 349 | MPTAASATESAIEDTPAPARPEMDGRTKPKRQPRYHVVLWDDDDHTYQYVVVMLRSLFGH |
| | | PPERGYRLAKEMDTQGRVIVLTTTREHAELKRDQIHAVGYDRLLARSKGSMKASIEAEE |
| PS998 | 350 | MPTAASATESAIEDTPAPARPEVDGRTKPKRHPRYHVVLWDDDDHTYQYVVVMLQSLFGH |
| | | SPKRGYCLAKEMDTQGRVIVLTTTREHAELKRDQIHAFGYDRLLARSKGSMKASIEAEE |
| PS999 | 351 | MPTAASATESAFEDTPAPARPEVDGRTKPKRQPRYHVVLWDDDDHTYQYVVVMLRSLFGY |
| | | PPERGYRLAKEMDTQGRVIVLTTTREHAELKRDQIHAFGYDRLLARSKGSMKASIEAEE |
| PS1000 | 352 | MPTAASATESAIEDTPAPARPVVDGRTKPKRQPRYHVVLWDDDDHTYQYVVVMLRSLFGH |
| | | PPERGYRLAKEMDTQGRVIVLTTTREHAELKRDQIHAFGYDRLLARSKGSMKASIEAEE |
| PS1001 | 353 | MPTAASATESAFEDTPAPARPEVDGRTKPKRQPRYHVVLWDDDDHTYQYVVVMLRTLFGH |
| | | PPERGYRLAKEMDTQGRVIVLTTTREHAELKRDQIHAFGYDRLLARSKGSMKASIEAEE |
| PS1002 | 354 | MPTAASATESAIEDTPAPARPEVDGRTKPKHQPRYHVVLWDDDDHTYQYVVVMLRDLFGH |
| | | PPERGYRLAKEMDTQGRVIVLTTTREHAELKRDQIHAFGYDRLLARSKGSMKASIEAEE |
| PS1003 | 355 | MPTAASATESAIEDTPAPARPEVDGRTKPKHQPRYHVVLWDDDDHTYQYVVVMLRSLFGH |
| | | PKQRGYRLAKEMDTQGRVIVLTTTREHAELKRDQIHAFGYDRLLARSKGSMKASIEAEE |
| PS1004 | 356 | MPTAASATESAIEDTPAPARPVVDGRTKPKHQPRYHVVLWDDDDHTYQYVVVMLRSLFGH |
| | | PQRRGYRLAKEMDTQGRVIVLTTTREHAELKRDQIHAFGYDRLLARSKGSMKASIEAEE |
| PS1005 | 357 | MPTAASATESAIEDTPAPARPVVDGRTKPKRQPRYHVVLWDDDDHTYQYVVVMLLSLFGH |
| | | PSERGYRLAKEMDTQGRVIVLTTTREHAELKRDQIHAFGYDRLLARSKGSMKASIEAEE |
| PS1006 | 358 | MPTAASATESAFEDTPAPARPEVDGRTKPKHQPRYHVVLWDDDDHTYQYVVVMLRSLFGH |
| | | PPERGYRLAKEMDTQGRVIVLTTTREHAELKRDQIHAFGYDRLLARSKGSMKASIEAEE |
| PS1007 | 359 | MPTAASATESAIEDTPAPARPEVDGRTKPKHQPRYHVVLWDDDDHTYQYVVVMLRYLFGH |
| | | PPERGYRLAKEMDTQGRVIVLTTTREHAELKRDQIHAFGYDRLLARSKGSMKASIEAEE |
| PS1008 | 360 | MPTAASATESAIEDTPALARPEVDGRTKPKHQPRYHVVLWDDDDHTYQYVVVMLLSLFGH |
| | | PPERGYRLAKEMDTQGRVIVLTTTREHAELKRDQIHAFGYDRLLARSKGSMKASIEAEE |
| PS1009 | 361 | MPTAASATESAIEDTPAPARPVVDGRTKPKRQPRYHVVLWDDHDHTYQYVVVMLRSLFGH |
| | | PPERGYRLAKEMDTQGRVIVLTTTREHAELKRDQIHAFGYDRLLARSKGSMKASIEAEE |
| PS1010 | 362 | MPTAASATESAIEDTPAPARPEVDGRTKPKRQPRYHVVLWDDDDHTYQYVVVMLRSLFGH |
| | | PPERGYRKAKELDTQGRVIVLTTTREHAELKRDQIHAFGYDRLLARSKGSMKASIEAEE |
| PS1011 | 363 | MPTAASATESAIEDTPAPARPEVDGRTKPKRQPRYHVVLWDDDDHTYQYVVVMLRSLFGH |
| | | PPERGYRLAKELVTQGRVIVLTTTREHAELKRDQIHAFGYDRLLARSKGSMKASIEAEE |
| PS1012 | 364 | MPTAASATESAIEDTPAPARPEVDGRTKPKRQPRYHVVLWDDDDHTYQYVVVMLRTLFGH |
| | | PPSRGYRMAKEMDTQGRVIVLTTTREHAELKRDQIHAFGRDRLLARSKGSMKASIEAEE |
| PS1013 | 365 | MPTAASATESAIEDTPAPARPEVDGRTKPKRQPRYHVVLWDDDDHTYQYVVVMLRDLFGH |
| | | PPSRGYRMAKEMDTQGRVIVLTTTREHAELKRDQIHAFGRDRLLARSKGSMKASIEAEE |
| PS1014 | 366 | MPTAASATESAIEDTPAPARPEVDGRTKPKRQPRYHVVLWDDDDHTYQYVVVMLRYLFGH |
| | | PPSRGYRMAKEMDTQGRVIVLTTTREHAELKRDQIHAFGRDRLLARSKGSMKASIEAEE |
| PS1015 | 367 | MPTAASATESAIEDTPAPARPEVDGRTKPKRQPRYHVVLWDDDDHTYQYVVVMLRSLFGH |
| | | PKQRGYRMAKEMDTQGRVIVLTTTREHAELKRDQIHAFGRDRLLARSKGSMKASIEAEE |
| PS1016 | 368 | MPTAASATESAFEDTPAPARPEVDGRTKPKHQPRYHVVLWDDDDHTYQYVVVMLRSLFGH |
| | | PPSRGYRMAKEMDTQGRVIVLTTTREHAELKRDQIHAFGRDRLLARSKGSMKASIEAEE |
| PS1017 | 369 | MPTAASATESAIEDTPAPARPEVDGRTKPKRQPRYHVVLWDDDDHTYQYVVVMLRSLFGH |
| | | PQRRGYRMAKEMDTQGRVIVLTTTREHAELKRDQIHAFGRDRLLARSKGSMKASIEAEE |
| PS1018 | 370 | MPTAASATESAIEDTPAPARPVVDGRTKPKHQPRYHVVLWDDDDHTYQYVVVMLRSLFGH |
| | | PQRRGYRMAKEMDTQGRVIVLTTTREHAELKRDQIHAFGRDRLLARSKGSMKASIEAEE |
| PS1019 | 371 | MPTAASATESAIEDTPAPARPEVDGRTKPKRQPRYHVVLWDDDDHTYQYVVVMLRSLFGH |
| | | PRSRGYRMAKEMDTQGRVIVLTTTREHAELKRDQIHAFGRDRLLARSKGSMKASIEAEE |
| PS1020 | 372 | MPTAASATESAFEDIPAPARPEVDGRTKPKRQPRYHVVLWDDDDHTYQYVVVMLRSLFGH |
| | | PPSRGYRMAKEMDTQGRVIVLTTTREHAELKRDQIHAFGRDRLLARSKGSMKASIEAEE |
| PS1021 | 373 | MPTATSATESAIEDTPAPARPEVDGRTKPKRQPHYHVVLWDDDDHTYQYVVVMLRSLFGH |
| | | PPSRGYRMAKEMDTQGRVIVLTTTREHAELKRDQIHAFGRDRLLARSKGSMKASIEAEE |
| PS1022 | 374 | MPTAASATESAIEDTPAPARPEVDGRTKPKRQPRYHVVLWDDDDHTYQYVVVMLRSLFGH |
| | | PRERGYRLAKEMDTQGRVIVLTTTREHAELKRDQIHAFGYDRLLARSKGSMKASIEAEE |
| PS1023 | 375 | MPTAASATESAFEDIPAPARPEVDGRTKPKRQPRYHVVLWDDDDHTYQYVVVMLRSLFGH |
| | | PPERGYRLAKEMDTQGRVIVLTTTREHAELKRDQIHAFGYDRLLARSKGSMKASIEAEE |
| PS1024 | 376 | MPTATSATESAIEDTPAPARPEVDGRTKPKRQPHYHVVLWDDDDHTYQYVVVMLRSLFGH |
| | | PPERGYRLAKEMDTQGRVIVLTTTREHAELKRDQIHAFGYDRLLARSKGSMKASIEAEE |
| PS1025 | 377 | MPTAASATESAIEDTPAPARPEVDGRTKPKKQPRYHVVLWDDDDHTYQYVVVMLRSLFGH |
| | | PPSRGYRMAKEMDTQGRVIVLTTTREHAELKRDQIHAFGRDRLLARSKGSMKASIEAEE |
| PS1026 | 378 | MPTAASATESAIEDTPAPARPEVDGRTKPKRQPRYHVVLWDDDDHTYQYVVVMLRSLFGF |
| | | PPSRGYRMAKEMDTQGRVIVLTTTREHAELKRDQIHAFGRDRLLARSKGSMKASIEAEE |
| PS1027 | 379 | MPTAASATESAIEDIPAPARPEVDGRTKPKRQPRYHVVLWDDDDHTYQYVVVMLRSLFGH |
| | | PPSRGYRMAKEMDTQGRVIVLTTTREHAELKRDQIHAFGRDRLLARSKGSMKASIEAEE |
| PS1028 | 380 | MPTAASATESAIEDTPAPARPEVDGRTKPKRQPRYHVVLWDDDDHTYQYVVVMFRSLFGH |
| | | PPSRGYRMAKEMDTQGRVIVLTTTREHAELKRDQIHAFGRDRLLARSKGSMKASIEAEE |
| PS1029 | 381 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHALGRDRLLARSKGSMKASIEAEE |
| PS1030 | 382 | MPTAASATESAIEDTPAPARPEVDGRTKPKRQPRYHVVLWDDDDHTYQYVVVMLRSLFGH |
| | | PPSRGYRMAKEMDTQGRVIVLTTTNEHAELKRDQIHAFGRDRLLARSKGSMKASIEAEE |
| PS1031 | 383 | MPTAASATESAIEDTPAPARPEVDGRTKPKRQPRYHVVLWDDDDHTYQYVVVMFRSLFGH |
| | | PPSRGYRMAKEMDTQGRVIVLTTTREHAELKRDQIHALGRDRLLARSKGSMKASIEAEE |
| PS1032 | 384 | MPTAASATESAIEDTPAPARPEVDGRTKPKRQPRYHVVLWDDDDHTYQYVVVMLRSLFGH |
| | | PPSRGYRMAKEMDTQGVVIVLTTTREHAELKRDQIHAFGRDRLLARSKGSMKASIEAEE |
| PS1033 | 385 | MPTAASATESAIEDTPAPARPEVDGRTKPKRQPRYHVVLWDDDDHTYQYVVVMLRSLFGH |
| | | PPSRGYRMAKEMRTQGRVIVLTTTREHAELKRDQIHAFGRDRLLARSKGSMKASIEAEE |
| PS1034 | 386 | MPTAASATESAIEDTPAPARPEVDGRTKPKRQPRYHVVLWDDDDHTYQYVVVMLRSLFGH |
| | | PPSRGYRMAKEMDTQGRVIPLTTTREHAELKRDQIHAFGRDRLLARSKGSMKASIEAEE |
| PS1035 | 387 | MPTAASATESAIEDTPAPARPEVDGRTKPKRQPRYHVVNWDDDDHTYQYVVVMLRSLFGH |
| | | PPSRGYRMAKEMDTQGRVIVLTTTREHAELKRDQIHAFGRDRLLARSKGSMKASIEAEE |
| PS1036 | 388 | MPTAASATESAIEDTPAPARPEVDGRTKPKRQPRYHVVLWDDDDHTYQYVVVMLRSLFGH |
| | | PPSRGYRMAKEMDTQGRVIVLTTTTEHAELKRDQIHAFGRDRLLARSKGSMKASIEAEE |
| PS1038 | 389 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAFGRDRLLARSKGSMKASIEAEEG |
| | | SAGSAAGSGEFMPTAASATESAIEDTPAPARPEVDGRTKPKRQPRYHVVLWDDDDHTYQY |
| | | VVVMLRSLFGHPPSRGYRMAKEMDTQGRVIVLTTTREHAELKRDQIHAFGRDRLLARSKG |
| | | SMKASIEAEE |
| PS1043 | 390 | MPTAASATESAIEDTPAPARPEVDGRTKPKRQPRYHVVLWDDDDHTYQYVVVMLRSLFGH |
| | | PKQRGYRMAKEMDTQGRVIVLTTTREHAELKRDQIHAFGRDRLLARSKGSMKASIEAEEG |
| | | SAGSAAGSGEFMPTAASATESAIEDTPAPARPEVDGRTKPKRQPRYHVVLWDDDDHTYQY |
| | | VVVMLRSLFGHPKQRGYRMAKEMDTQGRVIVLTTTREHAELKRDQIHAFGRDRLLARSKG |
| | | SMKASIEAEE |
| PS1044 | 391 | MSPILGYWKIKGLVQPTRLLLEYLEEKYEEHLYERDEGDKWRNKKFELGLEFPNLPYYID |
| PS1045 | 392 | MSPILGYWKIKGLVQPTRLLLEYLEEKYEEHLYERDEGDKWRNKKFELGLEFPNLPYYID |
| PS1046 | 393 | MGGLFFNALKNCKENFTVLQTIRQQQSTLNGSWVALLQTRNTLNRAGIRYMMDQNNIGSG |
| | | STVAELMESASISLKQAEKNWADYEALPRDPRQSTAAAAEIKRNYDIYHNALAELIQLLG |
| | | AGKINEFFDQPTQGYQDGFEKQYVAYMEQNDRLHDIAVSDNNASYS |
| PS1047 | 394 | MGGLFFNALKNDKENFTVLQTIRQQQSTLNGSWVALLQTRNTLNRAGIRYMMDQNNIGSG |
| | | STVAELMESASISLKQAEKNWADYEALPRDPRQSTAAAAEIKRNYDIYHNALAELIQLLG |
| | | AGKINEFFDQPTQGYQDGFEKQYVAYMEQNDRLHDIAVSDNNASYS |
| PS1048 | 395 | MPTAASATESAIEDTPAPARPEVDGRTKPKRQPRYHVVLWDDDDHTYQYYVVMLRSLFGH |
| | | PPSRGYRMAKEMDTQGRVIVLTTTREHAELKRDQIHAFGRDRLLARSKGSMKASIEAEE |
| PS1049 | 396 | MPTAASATESAIEDTPAPARPEVDGRTKPKRQPRYHVVLWDDDDHTYQYYVVMLRSLFGH |
| | | PPSRGYRMAKEIDTQGRVIVLTTTREHAELKRDQIHAFGRDRLLARSKGSMKASIEAEE |
| PS1050 | 397 | MPTAASATESAIEDTPAPARPEVDGRTKPKRQPRYHVVLWEDDDETYQYIVVMLRSLFGH |
| | | PPSRGYRMAKELDTQGRVIVLTTTREHAELKRDQIHAFGRDRLLARSKGSLKASIEAEE |
| PS1051 | 398 | MPTAASATESAIEDTPAPARPEVDGRTKPKRQPRYHVVLWNDDDLTYQYLVVMLRSLFGH |
| | | PPSRGYRMIKEMDTQGRVIVLTTTREHAELKRDQIHAFGRDRLLARSKGSPKASIEAEE |
| PS1052 | 399 | MPTAASATESAIEDTPAPARPEVDGRTKPKRQPRYHVVLWEDDDETYQYLVVMLRSLFGH |
| | | PPSRGYRMAKEIDTQGRVIVLTTTREHAELKRDQIHAFGRDRLLARSKGSMKASIEAEE |
| PS1053 | 400 | MPTAASATESAIEDTPAPARPEVDGRTKPKRQPRYHVVLWDDDDDTYQYLVVMLRSLFGH |
| | | PPSRGYRMMKEIDTQGRVIVLTTTREHAELKRDQIHAFGRDRLLARSKGSLKASIEAEE |
| PS1054 | 401 | MPTAASATESAIEDTPAPARPEVDGRTKPKRQPRYHVVLWDDDDETYQYLVVMLRSLFGH |
| | | PPSRGYRMVKEADTQGRVIVLTTTREHAELKRDQIHAFGRDRLLARSKGSMKASIEAEE |
| PS1055 | 402 | MPTAASATESAIEDTPAPARPEVDGRTKPKRQPRYHVVLWTDDDQTYQYMVVMLRSLFGH |
| | | PPSRGYRMAKELDTQGRVIVLTTTREHAELKRDQIHAFGRDRLLARSKGSPKASIEAEE |
| PS1056 | 403 | MPTAASATESAIEDTPAPARPEVDGRTKPKRQPRYHVVLWSDDDETYQYIVVMLRSLFGH |
| | | PPSRGYRMIKELDTQGRVIVLTTTREHAELKRDQIHAFGRDRLLARSKGSVKASIEAEE |
| PS1057 | 404 | MPTAASATESAIEDTPAPARPEVDGRTKPKRQPRYHVVLWDDDDDTYQYLVVMLRSLFGH |
| | | PPSRGYRMAKELDTQGRVIVLTTTREHAELKRDQIHAFGRDRLLARSKGSMKASIEAEE |
| PS1058 | 405 | MPTAASATESAIEDTPAPARPEVDGRTKPKRQPRYHVVLWDDDDHTYQYVVVMLRSLFGH |
| | | PPSRGYRMVKEMDTQGRVIVLTTTREHAELKRDQIHAFGRDRLLARSKGSVKASIEAEE |
| PS1059 | 406 | MPTAASATESAIEDTPAPARPEVDGRTKPKRQPRYHVVLWEDDDETYQYLVVMLRSLFGH |
| | | PPSRGYRMVKEIDTQGRVIVLTTTREHAELKRDQIHAFGRDRLLARSKGSWKASIEAEE |
| PS1060 | 407 | MHHHHHHHHHHDYDIPTTENLYFQGMPTAASATESAIEDTPAPARPEVDGRTKPKRQPRY |
| | | HVVLWDDDDHTYQYVVVMLRSLFGHPPSRGYRMAKEMDTQGRVIVLTTTREHAELKRDQI |
| | | HAFGRDRLLARSKGSMKASIEAEE |
| PS1061 | 408 | MPTAASATESAIEDTPAPARPEVDGRTEPKRQPRYHVVLWDDDDHTYQYVVVMLRSLFGH |
| | | PPSRGYRMAKEMDTQGRVIVLTTTREHAELKRDQIHAFGRDRLLARSKGSMKASIEAEE |
| PS1062 | 409 | MPTAASATESAIEDTPAPARSEVDGRTKPERQPRYHVVLWDDDDHTYQYVVVMLRSLFGH |
| | | PPSRGYRMAKEMDTQGRVIVLTTTREHAELKRDQIHAFGRDRLLARSKGSMKASIEAEE |
| PS1063 | 410 | MPTAASATESAIEDTPAPARPEVDGRTKPKRQPRYHVVLWDDDDHSYQYVVVMLRSLFGH |
| | | PPSRGYRMAKEMDTQGRVIVLTTTREHAELKRDQIHAFGRDRLLARSKGSMKASIEAEE |
| PS1064 | 411 | MPTAASATESAIEDTPAPARPEVDGRTKPKRQPRYHVVLWDDDDHIYQYVVVMLRSLFGH |
| | | PPSRGYRMAKEMDTQGRVIVLTTTREHAELKRDQIHAFGRDRLLARSKGSMKASIEAEE |
| PS1065 | 412 | MPTAASATESAIEDTPAPARPEVDGRTKPKRQPRYHVVLWDDDDHTYQYVVVMLRSLVGH |
| | | PPSRGYRMAKEMDTQGRVIVLTTTREHAELKRDQIHAFGRDRLLARSKGSMKASIEAEE |
| PS1066 | 413 | MPTAASATESAIEDTPAPARPEVDGRTKPKRQPRYHVVLWDDDDHTYQYVVVMLRSLFGH |
| | | SPSRGYRMAKEMDTQGRVIVLTTTREHAELKRDQIHAFGRDRLLARSKGSMKASIEAEE |
| PS1067 | 414 | MPTAASATESAIEDTPAPARPEVDGRTKPKRQPRYHVVLWDDDDHTYQYVVVMLRSLFGH |
| | | PPSRGYRMAKEMHTQGRVIVLTTTREHAELKRDQIHAFGRDRLLARSKGSMKASIEAEE |
| PS1068 | 415 | MPTAASATESAIEDTPAPARPEVDGRTKPKRQPRYHVVLWDDDDHTYQYVVVMLRSLFGH |
| | | PPSRGYRMAKEMVTQGRVIVLTTTREHAELKRDQIHAFGRDRLLARSKGSMKASIEAEE |
| PS1069 | 416 | MPTAASATESAIEDTPAPARPEVDGRTKPKRQPRYHVVLWDDDDHTYQYVVVMLRSLFGH |
| | | PPSRGYRMAKEMDTLGRVIVLTTTREHAELKRDQIHAFGRDRLLARSKGSMKASIEAEE |
| PS1070 | 417 | MPTAASATESAIEDTPAPARPEVDGRTKPKRQPRYHVVLWDDDDHTYQYVVVMLRSLFGH |
| | | PPSRGYRHAKTMDTQGRVIVLTTTREHAELKRDQIHALGRDRLLARSKGSMKASIEAEE |
| PS1071 | 418 | MPTAASATESAIEDTPAPARPEVDGRTKPKRQPRYHVVLWDDDRHTYQYVVVMLRSLFGH |
| | | PPSRGYRMAKEMDTQGRVIVLTTTREHAELKRDQIHAFGRDRLLARSKGSMKASIEAEE |
| PS1072 | 419 | MPTAASATESAIEDTPAPARPEVDGRTKPKRQPRYHVVLWDDDDHTDQYVVVMLRSLFGH |
| | | PPSRGYRMALEAHTQGRVIVLTTTREHAELKRDQIHAFGRDRLLARSKGSMKASIEAEE |
| PS1073 | 420 | MPTAASATESAIEDTPAPARPEVDGRTKPKRQPRYHVVLEDDDDHTYQYVVVMLRSLFGH |
| | | PPSRGYRMAKEYDTQGRVIVLTTTREHAELKRDQIHAFGRDRLLARSKGSMKASIEAEE |
| PS1074 | 421 | MPTAASATESAIEDTPAPARPEVDGRTKPKRQPRYHVVLWDDDDHTYQYIVVMLRSLFGH |
| | | PPSRGYRMARIMDTQGRVIVLTTTREHAELKRDQIHAFGRDRLLARSKGSMKASIEAEE |
| PS1075 | 422 | MPTAASATESAIEDTPAPARPEVDGRTKPKRQPRYHVVLWDDDDHTYQYVVVMLRSLFGH |
| | | PRQRGYRMAKEMDTQGRVIVLTTTREHAELKRDQIHAFGRDRLLARSKGSMKASIEAEE |
| PS1076 | 423 | MAIEDTPAPARPEVDGRTKPKRQPRYHVVLWDDDDHTYQYVVVMLRSLFGHPPSRGYRMA |
| | | KEMDTQGRVIVLTTTREHAELKRDQIHAFGRDRLLARSKGSMKASIEAEE |
| PS1077 | 424 | MPEVDGRTKPKRQPRYHVVLWDDDDHTYQYVVVMLRSLFGHPPSRGYRMAKEMDTQGRVI |
| | | VLTTTREHAELKRDQIHAFGRDRLLARSKGSMKASIEAEE |
| PS1078 | 425 | MPRYHVVLWDDDDHTYQYVVVMLRSLFGHPPSRGYRMAKEMDTQGRVIVLTTTREHAELK |
| | | RDQIHAFGRDRLLARSKGSMKASIEAEE |
| PS1079 | 426 | MPTAASATESAIEDTPAPARPEVDGRTKPKRQPRYHVVLWNDDDHTYQYVVVMLQSLFGH |
| | | PPERGYRLAKEVDTQGRVIVLTTTREHAELKRDQIHAFGYDPLIDRCKGSMSASIEAEE |
| PS1080 | 427 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAFGRDPLIDRCKGSMSASIEAEE |
| PS1082 | 428 | MPTAASATESAIEDTPAPARPEVDGRTKPKRQPRYHVVLWDDDDHTYQYIVVMLRSLFGH |
| | | PPSRGYRMAKEMDTQGRVIVLTTTREHAELKRDQIHAFGRDRLLARSKGSMKASIEAEE |
| PS1083 | 429 | MPTAASATESAIEDTPAPARPEVDGRTKPKRQPRYHVVLWDDDDHTYQYTVVMLRSLFGH |
| | | PPSRGYRMAKELDTQGRVIVLTTTREHAELKRDQIHAFGRDRLLARSKGSMKASIEAEE |
| PS1084 | 430 | MPTAASATESAIEDTPAPARPEVDGRTKPKRQPRYHVVLWNDDDHTYQYLVVMLRSLFGH |
| | | PPSRGYRMAKELDTQGRVIVLTTTREHAELKRDQIHAFGRDRLLARSKGSMKASIEAEE |
| PS1085 | 431 | MPTAASATESAIEDTPAPARPEVDGRTKPKRQPRYHVVLWEDDDHTYQYLVVMLRSLFGH |
| | | PPSRGYRMAKEYDTQGRVIVLTTTREHAELKRDQIHAFGRDRLLARSKGSNKASIEAEE |
| PS1086 | 432 | MPTAASATESAIEDTPAPARPEVDGRTKPKRQPRYHVVLWDDDDHTYQYVVVMLRSLFGH |
| | | PTERGYRLAKEMDTQGRVIVLTTTREHAELKRDQIHAFGRDRLLARSKGSMKASIEAEE |
| PS1087 | 433 | MPTAASATESAIEDTPAPARPEVDGRTKPKRQPRYHVVLWDDDDHTYQYVVVMLRSLFGH |
| | | PTERGYRMAKEMDTQGRVIVLTTTREHAELKRDQIHAFGRDRLLARSKGSMKASIEAEE |
| PS1088 | 434 | MPTAASATESAIEDTPAPARPEVDGRTKPKRQPRYHVVLWDDDDHTYQYVVVMLRSLFGH |
| | | PPWRGYRLAREMDTQGRVIVLTTTREHAELKRDQIHAFGYDRLLARSKGSMKASIEAEE |
| PS1089 | 435 | MPTAASATESAIEDTPAPARPEVDGRTKPKRQPRYHVVLWDDDDHTYQYVVVMLRSLFGH |
| | | PPWRGYRLAREMDTQGRVIVLTTTREHAELKRDQIHAFGRDRLLARSKGSMKASIEAEE |
| PS1090 | 436 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAFGRDRLLARSKGSLEASIEAEE |
| PS1091 | 437 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAFGRDRLLARSKGSLEASIEAEE |
| PS1092 | 438 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHASGRDRLLARSKGSYKASIEAEE |
| PS1093 | 439 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHASGRDRLLAHSKGSKKASIEAEE |
| PS1094 | 440 | MPTAASATESAIEDTPAPARPEVDGCTKPKRQPRYHVVLWDDDDHTYQYVVVMLQSLFGH |
| | | PPERGYRLAKEMDTQGCVIVLTTTREHAELKRDQIYAFGYDRLLARSKGSMKASIEAEE |
| PS1095 | 441 | MPTAASATESAIEDTPAPARPEVDGRTKPKRQPRYHVVLWDDDDHTYQYVVEMLRTLFGH |
| | | PPERGYRLAKEMDTQGRVIVLTTTREHAELKRDQIHAFGRDRLLARSKGSMKACIEAEE |
| PS1096 | 442 | MPTAASATESAIEDTPAPARPEVDGRAKPKRQPRYHVVLWNDDDHTYQYVVVVLQSLFSH |
| | | PRERGYRLAKEMDTQGRVIVLTTTREHAELKRDQIHAFGYDRLLARSKGSMKASIEAEE |
| PS1097 | 443 | MPTAASATESAIGDTPAPARPKMDGRTKPKRQPRYHVVLWNDDDHTYQYAVVMLQSLFGH |
| | | PPERGYRQAKEVDTQGRVIVLTTTREHAELKRDQIHAFGYDRLLARSKGSMKASIEAEE |
| PS1098 | 444 | MPTAASATESAIGDTPAPARPEVDGRTKPKRQPRYHVVLWDDDDHTYQYVVVMLRDLFGH |
| | | PPERGYHMAKEMDTQGRVIVLTTTREHAELKRDQIHAFGRDRLLARSKGSMKASIEAEE |
| PS1099 | 445 | MGSSHHHHHHSSGENLYFQGHMPTAASATESAIEDTPAPARPEVDGRTKPKRQPRYHVVL |
| | | WDDDDHTYQYVVVMLRSLFGHPPSRGYRMAKEMDTQGRVIVLTTTREHAELKRDQIHAFG |
| | | RDRLLARSKGSMKASIEAEE |
| PS1100 | 446 | MGSSHHHHHHSSGENLYFQGHMQPRYHVVLWDDDDHTYQYVVVMLRSLFGHPPSRGYRMA |
| | | KEMDTQGRVIVLTTTREHAELKRDQIHAFGRDRLLARSKGSMKASIEAEE |
| PS1101 | 447 | MHSKFSHAGRICGAKFKVREPIYRCKECSFDDTCVLCVNCFNPKDHTGHHVYTTICTEFN |
| | | NGICDCGDKEAWNHTLFCKAEEG |
| PS1102 | 448 | MHSKFSHAGRICGAKFKVGEPIYRCKECQFDDTCVLCVNCFNPKDHTGHHVYTTICTEFN |
| | | NGICDCGDKEAWNHTLFCKAEEG |
| PS1103 | 449 | MHSKFSHAGRICGAKFKVGEPIYRCKECSFDYTCVLCVNCFNPKDHTGHHVYTTICTEFN |
| | | NGICDCGDKEAWNHTLFCKAEEG |
| PS1104 | 450 | MHSKFSHAGRICGAKFKVGEPIYRCKECSFDDTCVLCVNCFNPKDHRGHHVYTTICTEFN |
| | | NGICDCGDKEAWNHTLFCKAEEG |
| PS1105 | 451 | MHSKFSHAGRICGAKFKVGEPIYRCKECSFDDTCVLCVNCFNPKDHTRHHVYTTICTEFN |
| | | NGICDCGDKEAWNHTLFCKAEEG |
| PS1106 | 452 | MHSKFSHAGRICGAKFKVGEPIYRCKECSFDDTCVLCVNCFNPKDHTGHHVYVTICTEFN |
| | | NGICDCGDKEAWNHTLFCKAEEG |
| PS1107 | 453 | MHSKFSHAGRICGAKFKVGEPIYRCKECSFDDTCVLCVNCFNPKDHTGHHVYTTICREFN |
| | | NGICDCGDKEAWNHTLFCKAEEG |
| PS1108 | 454 | MHSKFSHAGRICGAKFKVGEPIYRCKECSFDDTCVLCVNCFNPKDHTGHHVYTTICTERN |
| | | NGICDCGDKEAWNHTLFCKAEEG |
| PS1109 | 455 | MHSKFSHAGRICGAKFKVGEPIYRCKECSFDDTCVLCVNCFNPKDHTGHHVYTTICTEFN |
| | | KGICDCGDKEAWNHTLFCKAEEG |
| PS1110 | 456 | MHSKFSHAGRICGAKFKVGEPIYRCKECSFDDTCVLCVNCFNPKDHTGHHVYTTICTEFN |
| | | NGECDCGDKEAWNHTLFCKAEEG |
| PS1111 | 457 | MHSKFSHAGRICGAKFKVGEPIYRCKECSFDDTCVLCVNCFNPKDHTGHHVYTTICTEFN |
| | | NGICDCGDKSAWNHTLFCKAEEG |
| PS1112 | 458 | MHSKFSHAGRICGAKFKVGEPIYRCKECSFDDTCVLCVNCFNPKDHTGHHVYTTICTEFN |
| | | NGICDCGDKTAWNHTLFCKAEEG |
| PS1113 | 459 | MHSKFSHAGRICGAKFKVGEPIYRCKECSFDDTCVLCVNCFNPKDHTGHHVYTTICTEFN |
| | | NGICDCGDKEAWNKTLFCKAEEG |
| PS1114 | 460 | MHSKFSHAGRICGAKFKVGEPIYRCKECSFDDTCVLCVNCFNPKDHRGHHVYTTICTERN |
| | | NGICDCGDKEAWNHTLFCKAEEG |
| PS1115 | 461 | MHSKFSHAGRICGAKFKVGEPIYRCKECSFDDTCVLCVNCFNPKDHRGHHVYTTICTERN |
| | | NGICDCGDKEAWNHELFCKAEEG |
| PS1116 | 462 | MHSKFSHAGRICGAKFKVGEPIYRCKECSFDDTCVLCVNCFNPKDHTYHHVYTTICTEFN |
| | | NGICDCGDKEAWNHTLFCKAEEG |
| PS1117 | 463 | MHSKFSHAGRICGAKFKVGEPIYRCKECSFDDTCVLCVNCFNPKDHLGHHVYTTICTEFN |
| | | NGICDCGDKEAWNHTLFCKAEEG |
| PS1118 | 464 | MHSKFSHAGRICGAKFKVGEPIYRCKECSFDDTCVLCVNCFNPKDHTGHHVYTTICTKFN |
| | | NGICDCGDKEAWNHTLFCKAEEG |
| PS1119 | 465 | MHSKFSHAGRICGAKFKVGEPIYRCKECSFDDTCVLCVNCFNPKDHTGHHVYTTICKEFN |
| | | NGICDCGDKEAWNHTLFCKAEEG |
| PS1120 | 466 | MHSKFSHAGRICGAKFKVGEPIYRCKECSFDDTCVLCVNCFNPKDHTGHHVYTKICTEFN |
| | | NGICDCGDKEAWNHTLFCKAEEG |
| PS1121 | 467 | MHSKFSHAGRICGAKFKVGEPIYRCKECSFDDTCVLCVNCFNPKDHKGHHVYTTICTEFN |
| | | NGICDCGDKEAWNHTLFCKAEEG |
| PS1122 | 468 | MHSKFSHAGRICGAKFKVGEPIYRCKECSFDDTCVLCVNCFNPKDHLGHHVYTTICTEFN |
| | | NGECDCGDKTAWNHTLFCKAEEG |
| PS1123 | 469 | PLYQVVLLDDDDHTYDYIIEMLQQIFIFTMVEGYRRAEELERKGRSVLIVCELSEAEFAR |
| | | DQIPSYGSDWRLPHSQGSMSAVIEPAE |
| PS1124 | 470 | MPTAASATESAIEDTPAPARPEVDGRTKPKRQPRYHVVLWDDSDHTRQYAVVMLRSLFGH |
| | | PPSRGYRMAKEMATQGRVIVLTTTREHAELKRDQIHAFGRDRLLARSKGSMKASIEAEE |
| PS1125 | 471 | MPTAASATESAIEDTPAPARPEVDGRTKPKRQPRYHVVLWDDRDHTRQYAVVMLRSLFGH |
| | | PPSRGYRMAKEIRTQGRVIVLTTTREHAELKRDQIHAFGRDRLLARSKGSMKASIEAEE |
| PS1126 | 472 | MPTAASATESAIEDTPAPARPEVDGRTKPKRQPRYHVVLWNDRDHTSQYIVVMLRSLFGH |
| | | PPSRGYRMAKEMDTQGRVIVLTTTREHAELKRDQIHAFGRDRLLARSKGSMKASIEAEE |
| PS1127 | 473 | MPTAASATESAIEDTPAPARPEVDGRTKPKRQPRYHVVLWNDRDHTSQYIIVMLRSLFGH |
| | | PPSRGYRMAKELQTQGRVIVLTTTREHAELKRDQIHAFGRDRLLARSKGSMKASIEAEE |
| PS1128 | 474 | MPTAASATESAIEDTPAPARPEVDGRTKPKRQPRYHVVFWDDDDHTYQYVVVMLRSLFGH |
| | | PPSRGYRMAHEMCTQGRVIVLTTTREHAELKRDQIHAFGRDRLLARSKGSMKASIEAEE |
| PS1129 | 475 | MPTAASATESAIEDTPAPARPEVDGRTKPKRQPRYHVVLWDDDDHTYQYVVVMLRSLFYH |
| | | PPSRGYRMAKEMDTQGRVIVLTTTREHAELKRDQIHAFGRDRLLARSKGSMKASIEAEE |
| PS1130 | 476 | MPTAASATESAIEDTPAPARPEVDGRTKPKRQPRYHVVLWDDDDHTYQYVVVMLRSLFYH |
| | | PPSRGYRMAHEMDTQGRVIVLTTTREHAELKRDQIHAFGRDRLLARSKGSMKASIEAEE |
| PS1131 | 477 | MPTAASATESAIEDTPAPARPEVDGRTKPKRQPRYHVVQWDDDDHTYQYFVVMLRSLFGH |
| | | PPSRGYRMAKEMDTQGRVIVLTTTREHAELKRDQIHAFGRDRLLARSKGSMKASIEAEE |
| PS1132 | 478 | MPTAASATESAIEDTPAPARPEVDGRTKPKRQPRYHVVQIDDDDHTYQYVVVMLRSLFYH |
| | | PPSRGYRMAKEMDTQGRVIVLTTTREHAELKRDQIHAFGRDRLLARSKGSMKASIEAEE |
| PS1133 | 479 | MPTAASATESAIEDTPAPARPEVDGRTVPQRQPRYHVVLWDDDDHTYQYVVGMLRSLFGH |
| | | PPSRGYRMAKEMDTQGRVIVLTTTREHAELKRDQIHAFGRDRLLARSKGSMKASIEAEE |
| PS1134 | 480 | MPTAASATESAIEDIPAPARPEVDGRTKPKHQPRYHVVLWDDDDHTYQYVVVMLRSLFGH |
| | | PPSRGYRMASEMDTQGRVIVLTTTREHAELKRDQIHAFGRDRLLARSKGSMKASIEAEE |
| PS1135 | 481 | MPTAASATESAIEDTPAPARTEVDGRTVPKRQPRYHVVLWDDDDHTYQYVVEMLRSLFGH |
| | | PPSRGYRMAKEMDTQGRVIVLTTTREHAELKRDQIHAFGRDRLLARSKGSMKASIEAEE |
| PS1136 | 482 | MPTAASATESAIEDTPAPARPEVDGRTRPKRQPRYHVVLWDDDDHTYQYVVVMLRKLFGH |
| | | PPSRGYRMAKEMDTQGRVIVLTTTREHAELKRDQIHAFGRDRLLARSKGSMKASIEAEE |
| PS1137 | 483 | MPTAASATESAIEDTPAPARSEVDGYTVPKRQPRYHVVLWDDDDHTYQYVVVMLRSLFGH |
| | | PPSRGYRMAKEMDTQGRVIVLTTTREHAELKRDQIRAFGRDRLLARSKGSMKASIEAEE |
| PS1138 | 484 | MSGSKFRGHQKSKGNSYDVEVVLQHVDTGNSYLCGYLKIKGLTEEYPTLTVFFEGEIISK |
| | | KHPFLTRKWDADEDVDRKHWGKFLAFYQYAKSFNSDDFDYEELKNGDYVFMRWKEQFLVP |
| | | DHTIKDISGLSFAGFYYICFQKSAASIEGYYYHRSSEWYQSLNLTHV |
| PS1139 | 485 | MSGSKFRGHQKSKGNSYDVEVVLQHVDTGNSYLCGYLKIKGLTEEYPTLTVFFEGEIISK |
| | | KHPFLTRKWDADEDVDRKHWGKFLAFYQYAKSFNSDDFDYEELKNGDYVFMRWKEQFLVP |
| | | DHTIKDISGASFAGFYYICFQKSAASIEGYYYHRSSEWYQSLNLTHV |
| PS1140 | 486 | MSGSKFRGHQKSKGNSYDVEVVLQHVDTGNSYLCGYLKIKGLTEEYPTLTAFFEGEIISK |
| | | KHPFLTRKWDADEDVDRKHWGKFLAFYQYAKSFNSDDFDYEELKNGDYVFMRWKEQFLVP |
| | | DHTIKDISGLSFAGFYYICFQKSAASIEGYYYHRSSEWYQSLNLTHV |
| PS1141 | 487 | MPTAASATESAIEDTPAPARPEVDGRTKPKRQPRYHVVLWDDDDHTYQYVVVMLRSLFGH |
| | | PPSRGYRMAHEMDTQGRVIVLTTTREHAELKRDQIHAFGRDRLLARSKGSMKASIEAEE |
| PS1142 | 488 | MPTAASATESAIEDTPAPARPEVDGRTKPKRQPRYHVVLISDDDHTYQYTVVMLRSLFGH |
| | | PPSRGYRMAHEMDTQGRVIVLTTTREHAELKRDQIHAFGRDRLLARSKGSMKASIEAEE |
| PS1143 | 489 | MPTAASATESAIEDTPAPARPEVDGRTKPKRQPRYHVVFWDDDDHTYQYTVVMLRSLFYH |
| | | PPSRGYRMAHEMCTQGRVIVLTTTREHAELKRDQIHAFGRDRLLARSKGSMKASIEAEE |
| PS1144 | 490 | MPTAASATESAIEDTPAPARPEVDGRTKPKRQPRYHVVFWDDDDHTYQYTVVMLRSLFYH |
| | | PPSRGYRMAHEMDTQGRVIVLTTTREHAELKRDQIHAFGRDRLLARSKGSMKASIEAEE |
| PS1145 | 491 | MPTAASATESAIEDTPAPARPEVDGRTKPKRQPRYHVVLWDDDDHTYQYVVVMLRSLFGH |
| | | PPSRGYRMAKEMDTQGRFIVLTTTREHAELKRDQIHAFGRDRLLARSKGSMKASIEAEE |
| PS1146 | 492 | MPTAASATESAIEDTPAPARPEVDGRTKPKRQPRYHVVEWDDDSHTYQYVVVMLRSLFGH |
| | | PPSRGYRMDKEMDTQGRVIVLTTTREHAELKRDQIHAFGRDRLLARSKGSEKASIEAEE |
| PS1147 | 493 | MPTAASATESAIEDTPAPARPEVDGRTKPKRQPRYHVVLWDDVDHTYQYTVVMLRSLFGY |
| | | PPSRGYRMAKEVDTQGRVIVLTTTREHAELKRDQIHAFGRDRLLARSKGSMKASIEAEE |
| PS1148 | 494 | MPTAASATESAIEDTPAPARPEVDGRTKPKRQPRYHVVLWDDDDRTYQYVVVMLRSLFGH |
| | | PPSRGYRMDKEMDTQGRVIVLTTTREHAELKRDQIHAFGRDRLLARSKGSMKASIEAEE |
| PS1149 | 495 | MPTAASATESAIEDTPAPARPEVDGRTKPKRQPRYHVVLWDDKDHTPQYVVVMLRSLFGH |
| | | PPSRGYRMAKEMDTQGRVIVLTTTREHAELKRDQIHAFGRDRLLARSKGSMKASIEAEE |
| PS1150 | 496 | MPTAASATESAIEDTPAPARPEVDGRTKPKRQPRYHVVLWPDDDHTYQYVVVMLRSLFGH |
| | | PPSRGYRLAKEMDTQGRVIVLTTTREHAELKRDQIHAFGRDRLLARSKGSMKASIEAEE |
| PS1151 | 497 | MPTAASATESAIEDTPAPARPEVDGRTKPKRQPRYHVVLWDDDDCTYQYLVVMLRSLFGH |
| | | PPSRGYREAKEMDTQGRRIVLTTTREHAELKRDQIHAFGRDRLLARSKGSMKASIEAEE |
| PS1152 | 498 | MPTAASATESAIEDTPAPARPEVDGRTKPKRQPRYHVVLWDDDDHTYQYVVVMLRSLFGH |
| | | PPSRGYRMIKEMDTQGRVIVLTTTREHAELKRDQIHAFGRDRLLARSKGSMKASIEAEE |
| PS1153 | 499 | MPTAASATESAIEDTPAPARPEVDGRTKPKRQPRYHVVLWDDDDHTYQYVVVMLRSLFGI |
| | | PPSRGYRMAKEMDTQGRVIVLTTTREHAELKRDQIHAFGRDRLLARSKGSMKASIEAEE |
| PS1154 | 500 | MPTAASATESAIEDTPAPARPEVDGRTKPKRQPRYHVVLWDDDDHTYQYVVEMLRSLFGI |
| | | PPSRGYRMAKEMDTQGRVIVLTTTREHAELKRDQIHAFGRDRLLARSKGSMKASIEAEE |
| PS1155 | 501 | MPTAASATESAIKDTPAPARSEVDGRTKPERQPRYHVVLWDDDDHTYQYVVVMLRSLFGH |
| | | PTSRGYRMAKEMDTQGRVIVLTTTREHAELKRDQIHAFGRDRLLARSKGSMKASIEAEE |
| PS1156 | 502 | MPTAASATGSAIEDTPAPARPEVDGRTKPKRQPRYHVVLWDDDDHTYQYVVVMLRYLFGH |
| | | PPERGYRMAKEMDTQGRVIVLTTTREHAELKRDQIHAFGRDRLLARSKGSMKASIEAEE |
| PS1157 | 503 | MPTAASATESAIEDTPAPARPEVDGRTKPKRQPRYHVVLWSDEDNTKQYIVVMLRSLFGH |
| | | PPSRGYRMVEELDTQGRVIVLTTTREHAELKRDQIHAFGRDRLLARSKGSIKASIEAEE |
| PS1158 | 504 | MPTAASATESAIEDTPAPARPEVDGRTKPKRQPRYHVVVWDDDDNDEDYVVVMLRSLFGH |
| | | PPSRGYRMAKEMHTQGRVIVLTTTREHAELKRDQIHAFGRDRLLARSKGSLKASIEAEE |
| PS1159 | 505 | MPTAASATESAIEDTPAPARPEVDGRTKPKRQPRYHVVLWDDEDHTYDYIVVMLRSLFGH |
| | | PPSRGYRMAKELDTQGRVIVLTTTREHAELKRDQIHAFGRDRLLARSKGSVKASIEAEE |
| PS1160 | 506 | MPTAASATESAIEDTPAPARPEVDGRTKPKRQPRYHVVLWDDDDHTRQYLVVMLRSLFGH |
| | | PPSRGYRMTEEADTQGRVIVLTTTREHAELKRDQIHAFGRDRLLARSKGSMKASIEAEE |
| PS1161 | 50 | MPTAASATESAIEDTPAPARPEVDGRTKPKRQPRYHVVIWDDEDHTHDYWVVMLRSLFGH |
| | | PPSRGYRMSEELDTQGRVIVLTTTREHAELKRDQIHAFGRDRLLARSKGSMKASIEAEE |
| PS1162 | 508 | MGDVQPETCRPSAASGNYFPQYPEYAIETARLRTFEAWPRNLKQKPHQLAEAGFFYTGVG |
| | | DRVRCFSCGGGLMDWNDNDEPWEQHARHLSQCRFVKLMKGQLYIDTVAAKPVLAEEKEES |
| | | TSIGGD |
| PS1163 | 509 | MGSDAVSSDRNFPNSTNLPRNPSMADYEARIFTFGTWIYSVNKEQLARAGFYALGEGDKV |
| | | KCFHCGGGLTDWKPSEDPWEQHAKWYPGCKYLLEQKGQEYINNIHLTHSLEECLVR |
| PS1164 | 510 | MGSDAVSSDRNFPNSTNLPRNPSMADYEARIFTFGTWIYSVNKEQLARAGFYALGEGDKV |
| | | KCFHCGGGLTDWKPSEDPWEQHARHYPGCKYLLEQKGQEYINNIHLTHSLEECLVR |
| PS1165 | 511 | MGLENSLETLRFSISNLSMQTHAARMRTFMYWPSSVPVQPEQLASAGFYYVGRNDDVKCF |
| | | CCDGGLRCWESGDDPWVEHAKWFPRCEFLIRMKGQEFVDEIQGRYPHLLEQLLSG |
| PS1166 | 512 | MGLENSLETLRFSISNLSMQTHAARMRTFMYWPSSVPVQPEQLASAGFYYVGRNDDVKCF |
| | | CCDGGLRCWESGDDPWVEHARHFPRCEFLIRMKGQEFVDEIQGRYPHLLEQLLSG |
| PS1167 | 513 | MGSMRYTVSNLSMQTHAARFKTFFNWPSSVLVNPEQLASAGFYYVGNSDDVKCFCCDGGL |
| | | RCWESGDDPWVQHAKWFPRCEYLIRIKGQEFIRQVQAS |
| PS1168 | 514 | MGSMRYTVSNLSMQTHAARFKTFFNWPSSVLVNPEQLASAGFYYVGNSDDVKCFCCDGGL |
| | | RCWESGDDPWVQHARHFPRCEYLIRIKGQEFIRQVQAS |
| PS1169 | 515 | MGSHMLETEEEEEEGAGATLSRGPAFPGMGSEELRLASFYDWPLTAEVPPELLAAAGFFH |
| | | TGHQDKVRCFFCYGGLQSWKRGDDPWTEHAKWFPSCQFLLRSKGRDFVHSVQETHSQLLG |
| | | SWDP |
| PS1170 | 516 | MGSHMLETEEEEEEGAGATLSRGPAFPGMGSEELRLASFYDWPLTAEVPPELLAAAGFFH |
| | | TGHQDKVRCFFCYGGLQSWKRGDDPWTEHARHFPSCQFLLRSKGRDFVHSVQETHSQLLG |
| | | SWDP |
| PS1171 | 517 | MGSHMSTNLPRNPSMTGYEARLITFGTWMYSVNKEQLARAGFYAIGQEDKVQCFHCGGGL |
| | | ANWKPKEDPWEQHAKWYPGCKYLLEEKGHEYINNIHLTRSLEGALVQTT |
| PS1172 | 518 | MGSHMSTNLPRNPSMTGYEARLITFGTWMYSVNKEQLARAGFYAIGQEDKVQCFHCGGGL |
| | | ANWKPKEDPWEQHARHYPGCKYLLEEKGHEYINNIHLTRSLEGALVQTT |
| PS1173 | 519 | MGSHMRYQEEEARLASFRNWPFYVQGISPCVLSEAGFVFTGKQDTVQCFSCGGCLGNWEE |
| | | GDDPWKEHAKWFPKCEFLRSKKSSEEITQYIQSYK |
| PS1174 | 520 | MGSHMRYQEEEARLASFRNWPFYVQGISPCVLSEAGFVFTGKQDTVQCFSCGGCLGNWEE |
| | | GDDPWKEHARHFPKCEFLRSKKSSEEITQYIQSYK |
| PS1175 | 521 | MPTAASATESAIEDTPAPARPEVDGRTKPKRQPRYHVVLWDDDDHTVQYIVVMLRSLFGH |
| | | PPSRGYRMAKEMHTQGRVIVLTTTREHAELKRDQIHAFGRDRLLARSKGSLKASIEAEE |
| PS1176 | 522 | MPTAASATESAIEDTPAPARPEVDGRTKPKRQPRYHVVLWDDDDHTVQYIVVMLRSLFGH |
| | | PPSRGYRMAKEATTQGRVIVLTTTREHAELKRDQIHAFGRDRLLARSKGSAKASIEAEE |
| PS1177 | 523 | MPTAASATESAIEDTPAPARPEVDGRTKPKRQPRYHVVLWDDEDHTMQYIVVMLRSLFGH |
| | | PPSRGYRMAKEMHTQGRVIVLTTTREHAELKRDQIHAFGRDRLLARSKGSAKASIEAEE |
| PS1178 | 524 | MPTAASATESAIEDTPAPARPEVDGRTKPKRQPRYHVVLWDDDDHTVQYIVVMLRSLFGH |
| | | PPSRGYRMAKEIGTQGRVIVLTTTREHAELKRDQIHAFGHDRLLARSKGSAKASIEAEE |
| PS1179 | 525 | MPTAASATESAIEDTPAPARPEVDGRTKPKRQPRYHVVLWDDDDHTVQYIVVMLRSLFGH |
| | | PPSRGYRMAKEMHTQGRVIVLTTTREHAELKRDQIHAFGHDRLLARSKGSAKASIEAEE |
| PS1180 | 526 | MPTAASATESAIEDTPAPARPEVDGRTKPKRQPRYHVVLWDDDDHTVQYIVVMLRSLFGH |
| | | PPSRGYRMAKEIYTQGRVIVLTTTREHAELKRDQIHAFGRDRLLARSKGSAKASIEAEE |
| PS1181 | 527 | MPTAASATESAIEDTPAPARPEVDGRTKPKRQPRYHVVLWDDDDHTVQYIVVMLRSLFGH |
| | | PPSRGYRMAKEMHTQGRVIVLTTTREHAELKRDQIHAFGRDRLLARSKGSAKASIEAEE |
| PS1182 | 528 | MPTAASATESAIEDTPAPARPEVDGRTKPKRQPRYHVVLWDDRDHTYQYIVVMLRSLFGH |
| | | PPSRGYRMAKEAYTQGRVIVLTTTREHAELKRDQIHAFGRDRLLARSKGSLKASIEAEE |
| PS1183 | 529 | MPTAASATESAIEDTPAPARPEVDGRTKPKRQPRYHVVLWDDRDHTAQYAVVMLRSLFGH |
| | | PPSRGYRMAKEIYTQGRVIVLTTTREHAELKRDQIHAFGHDRLLARSKGSAKASIEAEE |
| PS1184 | 530 | MPTAASATESAIEDTPAPARPEVDGRTKPKRQPRYHVVLWDDNDHTLQYIVVMLRSLFGH |
| | | PPSRGYRMAKEVDTQGRVIVLTTTREHAELKRDQIHAFGRDRLLARSKGSMKASIEAEE |
| PS1185 | 531 | MPTAASATESAIEDTPAPARPEVDGRTKPKRQPRYHVVLWDDDDHTVQYIVVMLRSLFGH |
| | | PPSRGYRMAKELDTQGRVIVLTTTREHAELKRDQIHAFGRDRLLARSKGSMKASIEAEE |
| PS1186 | 532 | MPTAASATESAIEDTPAPARPEVDGRTKPKRQPRYHVVLWDDGDHTWQYIVVMLRSLFGH |
| | | PPSRGYRMAKELTTQGRVIVLTTTREHAELKRDQIHAFGRDRLLARSKGSMKASIEAEE |
| PS1187 | 533 | MPTAASATESAIEDTPAPARPEVDGRTKPKRQPRYHVVLWDDSDHTFQYIVVMLRSLFGH |
| | | PPSRGYRMAKELHTQGRVIVLTTTREHAELKRDQIHAFGRDRLLARSKGSMKASIEAEE |
| PS1188 | 534 | MPTAASATESAIEDTPAPARPEVDGRTKPKRQPRYHVVLWDDADHTYQYIVVMLRSLFGH |
| | | PPSRGYRMAKEMHTQGRVIVLTTTREHAELKRDQIHAFGRDRLLARSKGSMKASIEAEE |
| PS1189 | 535 | MPTAASATESAIEDTPAPARPEVDGRTKPKRQPRYHVVLWDDDDHTMQYIVVMLRSLFGH |
| | | PPSRGYRMAKEVTTQGRVIVLTTTREHAELKRDQIHAFGRDRLLARSKGSMKASIEAEE |
| PS1190 | 536 | MPTAASATESAIEDTPAPARPEVDGRTKPKRQPRYHVVLWDDSDHTIQYIVVMLRSLFGH |
| | | PPSRGYRMAKEADTQGRVIVLTTTREHAELKRDQIHAFGRDRLLARSKGSMKASIEAEE |
| PS1191 | 537 | MPTAASATESAIEDTPAPARPEVDGRTKPKRQPRYHVVLWDDDDHTWQYIVVMLRSLFGH |
| | | PPSRGYRMAKEMHTQGRVIVLTTTREHAELKRDQIHAFGRDRLLARSKGSMKASIEAEE |
| PS1192 | 538 | MPTAASATESAIEDTPAPARPEVDGRTKPKRQPRYHVVLWDDDDHTVQYIVVMLRSLFGH |
| | | PPSRGYRMAKEMHTQGRVIVLTTTREHAELKRDQIHAFGRDRLLARSKGSMKASIEAEE |
| PS1193 | 539 | MPTAASATESAIEDTPAPARPEVDGRTKPKRQPRYHVVLWDDNDHTLQYIVVMLRSLFGH |
| | | PPSRGYRMAKEMHTQGRVIVLTTTREHAELKRDQIHAFGRDRLLARSKGSMKASIEAEE |
| PS1194 | 540 | MPTAASATESAIEDTPAPARPEVDGRTKPKRQPRYHVVLWDDDDHTVQYIVVMLRSLFGH |
| | | PPSRGYRMAKEISTQGRVIVLTTTREHAELKRDQIHAFGRDRLLARSKGSMKASIEAEE |
| PS1195 | 541 | MPTAASATESAIEDTPAPARPEVDGRTKPKRQPRYHVVLWDDGDHTLQYIVVMLRSLFGH |
| | | PPSRGYRMAKEMHTQGRVIVLTTTREHAELKRDQIHAFGRDRLLARSKGSMKASIEAEE |
| PS1196 | 542 | MPTAASATESAIEDTPAPARPEVDGRTKPKRQPRYHVVLWDDDDHTIQYIVVMLRSLFGH |
| | | PPSRGYRMAKEMHTQGRVIVLTTTREHAELKRDQIHAFGRDRLLARSKGSMKASIEAEE |
| PS1197 | 543 | MPTAASATESAIEDTPAPARPEVDGRTKPKRQPRYHVVLWDDEDHTMQYIVVMLRSLFGH |
| | | PPSRGYRMAKEMHTQGRVIVLTTTREHAELKRDQIHAFGRDRLLARSKGSLKASIEAEE |
| PS1198 | 544 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAFGRDRLLARSKGSMKASIEAEEG |
| | | SAGSAAGSGEFMDKDCEMKRTTLDSPLGKLELSGCEQGLHEIKLLGKGTSAADAVEVPAP |
| | | AAVLGGPEPLMQATAWLNAYFHQPEAIEEFPVPALHHPVFQQESFTRQVLWKLLKVVKFG |
| | | EVISYQQLAALAGNPAATAAVKTALSGNPVPILIPCHRVVSSSGAVGGYEGGLAVKEWLL |
| | | AHEGHRLGKPGLG |
| PS1199 | 545 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAFGRDRLLARSKGSMKASIEAEEG |
| | | SAGSAAGSGEFMPTAASATESAIEDTPAPARPEVDGRTKPKRQPRYHVVLWDDDDHTYQY |
| | | VVVMLRSLFGHPPSRGYRMAKEMDTQGRVIVLTTTREHAELKRDQIHAFGRDRLLARSKG |
| | | SMKASIEAEEGSAGSAAGSGEFMDKDCEMKRTTLDSPLGKLELSGCEQGLHEIKLLGKGT |
| | | SAADAVEVPAPAAVLGGPEPLMQATAWLNAYFHQPEAIEEFPVPALHHPVFQQESFTRQV |
| | | LWKLLKVVKFGEVISYQQLAALAGNPAATAAVKTALSGNPVPILIPCHRVVSSSGAVGGY |
| | | EGGLAVKEWLLAHEGHRLGKPGLG |
| PS1200 | 546 | MSDSPVDLKPKPKVKPKLERPKLYKVMLLNDDYTPMSFVTVVLKAVFRMSEDTGRRVMMT |
| | | AHRFGSAVVVVCERDIAETKAKEATDLGKEAGFPLMFTTEPEEGSAGSAAGSGEFMSDSP |
| | | VDLKPKPKVKPKLERPKLYKVMLLNDDYTPMSFVTVVLKAVFRMSEDTGRRVMMTAHRFG |
| | | SAVVVVCERDIAETKAKEATDLGKEAGFPLMFTTEPEEGHHHHHHHHHHGGGSGGGSGGG |
| | | SGLNDFFEAQKIEWHEGGGSGGGSGGGSGLNDFFEAQKIEWHEGSAGSAAGSGEFMDKDC |
| | | EMKRTTLDSPLGKLELSGCEQGLHEIKLLGKGTSAADAVEVPAPAAVLGGPEPLMQATAW |
| | | LNAYFHQPEAIEEFPVPALHHPVFQQESFTRQVLWKLLKVVKFGEVISYQQLAALAGNPA |
| | | ATAAVKTALSGNPVPILIPCHRVVSSSGAVGGYEGGLAVKEWLLAHEGHRLGKPGLG |
| PS1201 | 547 | MHSKFSHAGRICGAKFKVGEPIYRCKECSFDDTCVLCVNCFNPKDHTGHHVYTTICTEFN |
| | | NGICDCGDKEAWNHTLFCKAEEGGSAGSAAGSGEFMDKDCEMKRTTLDSPLGKLELSGCE |
| | | QGLHEIKLLGKGTSAADAVEVPAPAAVLGGPEPLMQATAWLNAYFHQPEAIEEFPVPALH |
| | | HPVFQQESFTRQVLWKLLKVVKFGEVISYQQLAALAGNPAATAAVKTALSGNPVPILIPC |
| | | HRVVSSSGAVGGYEGGLAVKEWLLAHEGHRLGKPGLG |
| PS1202 | 548 | MHSKFSHAGRICGAKFKVGEPIYRCKECSFDDTCVLCVNCFNPKDHTGHHVYTTICTEFN |
| | | NGICDCGDKEAWNHTLFCKAEEGGSAGSAAGSGEFMHSKFSHAGRICGAKFKVGEPIYRC |
| | | KECSFDDTCVLCVNCFNPKDHTGHHVYTTICTEFNNGICDCGDKEAWNHTLFCKAEEGGS |
| | | AGSAAGSGEFMDKDCEMKRTTLDSPLGKLELSGCEQGLHEIKLLGKGTSAADAVEVPAPA |
| | | AVLGGPEPLMQATAWLNAYFHQPEAIEEFPVPALHHPVFQQESFTRQVLWKLLKVVKFGE |
| | | VISYQQLAALAGNPAATAAVKTALSGNPVPILIPCHRVVSSSGAVGGYEGGLAVKEWLLA |
| | | HEGHRLGKPGLG |
| PS1203 | 549 | MPTAASATESAIEDTPAPARPEVDGRTKPKRQPRYHVVLLDDDDHTSQYVVVMLRSLFGH |
| | | PPSRGYRMSKEMDTQGRAIVLTTTREHAELKRDQIHAFGRDRLLARSKGSMKASIEAEE |
| PS1204 | 550 | MPTAASATESAIEDTPAPARPEVDGRTKPKRQPRYHVVLLDDPDHTYQYVVVMLRSLFGH |
| | | PPSRGYRMAKEMDTQGRVIVLTTTREHAELKRDQIHAFGRDRLLARSKGSMKASIEAEE |
| PS1205 | 551 | MPTAASATESAIEDTPAPARPEVDGRTKPKRQPRYHVVQWDDDDHTYQYVVVMLRSLFYH |
| | | PPSRGYRMAHEMDTQGRVIVLTTTREHAELKRDQIHAFGRDRLLARSKGSMKASIEAEE |
| PS1206 | 552 | MPTAASATESAIEDTPAPARPEVDGRTKPKRQPRYHVVLWDDDDHTYQYVVVMLRSLFGH |
| | | PPSRGYRMAKPMDTQGRVIVLTTTREHAELKRDQIHAFGRDRLLARSKGSMKASIEAEE |
| PS1207 | 553 | MPTAASATESAIEDTPAPARPEVDGRTKPKRQPRYHVVLWDDDDHTYQYVVVMLRSLFGH |
| | | PPSRGYRMAKEMKTQGRVIVLTTTREHAELKRDQIHAFGRDRLLARSKGSMKASIEAEE |
| PS1208 | 554 | MPTAASATESAIEDTPAPARPEVDGRTKPKRQPRYHVVLYDDDDHTYQYVVVMLRSLFGH |
| | | PPSRGYRMAKEMDTQGRVIVLTTTREHAELKRDQIHAFGRDRLLARSKGSMKASIEAEE |
| PS1209 | 553 | MPTAASATESAIEDTPAPARPEVDGRTKPKRQPRYHVVLQDDDDHTYQYVVVMLRSLFGH |
| | | PPSRGYRMAKEMDTQGRVIVLTTTREHAELKRDQIHAFGRDRLLARSKGSMKASIEAEE |
| PS1210 | 556 | MPTAASATESAIEDTPAPARPEVDGRTKPKRQPRYHVVLWDDGDHTSQYVVVMLRSLFGH |
| | | PPSRGYRMAKEMDTQGRVIVLTTTREHAELKRDQIHAFGRDRLLARSKGSMKASIEAEE |
| PS1211 | 557 | MPTAASATESAIEDTPAPARPEVDGRTKPKRQPRYHVVLWDDDTHTYQYVVVMLRSLFGH |
| | | PPSRGYRMAKEMDTQGRVIVLTTTREHAELKRDQIHAFGRDRLLARSKGSMKASIEAEE |
| PS1212 | 558 | MPTAASATESAIEDTPAPARPEVDGRTKPKRQPRYHVVLWNDSDHTGQYIVVMLRSLFGH |
| | | PPSRGYRMAKEKDTQGRVIVLTTTREHAELKRDQIHAFGRDRLLARSKGSMKASIEAEE |
| PS1213 | 559 | MPTAASATESAIEDTPAPARPEVDGRTKPKRQPRYHVVLWNDLDHTYQYIVVMLRSLFGH |
| | | PPSRGYRMAKEMHTQGRVIVLTTTREHAELKRDQIHAFGRDRLLARSKGSMKASIEAEE |
| PS1214 | 560 | MPTAASATESAIEDTPAPARPEVDGRTKPKRQPRYHVVLWNDGDHTWQYIVVMLRSLFGH |
| | | PPSRGYRMAKEMHTQGRVIVLTTTREHAELKRDQIHAFGRDRLLARSKGSMKASIEAEE |
| PS1215 | 561 | MPTAASATESAIEDTPAPARPEVDGRTKPKRQPRYHVVLWNDGDHTWQYIVVMLRSLFGH |
| | | PPSRGYRMAKEAKTQGRVIVLTTTREHAELKRDQIHAFGRDRLLARSKGSMKASIEAEE |
| PS1216 | 562 | MPTAASATESAIEDTPAPARPEVDGRTKPKRQPRYHVVLWDDGDHTVQYIVVMLRSLFGH |
| | | PPSRGYRMAKEMHTQGRVIVLTTTREHAELKRDQIHAFGRDRLLARSKGSMKASIEAEE |
| PS1217 | 563 | MPTAASATESAIEDTPAPARPEVDGRTKPKRQPRYHVVLWDDQDHTWQYIVVMLRSLFGH |
| | | PPSRGYRMAKEMHTQGRVIVLTTTREHAELKRDQIHAFGRDRLLARSKGSMKASIEAEE |
| PS1218 | 564 | MHSKFSHAGRICGAKFKVGEPIYRCKECSFDDTCVLCVNCFNPKDHRGHHVYTTICTERN |
| | | NGICDCGDKEAWNHELFCKAEEGSAGSAAGSGEFMHSKFSHAGRICGAKFKVGEPIYRCK |
| | | ECSFDDTCVLCVNCFNPKDHRGHHVYTTICTERNNGICDCGDKEAWNHELFCKAEEG |
| PS1219 | 565 | MHSKFSHAGRICGAKFKVGEPIYRCKECSFDDTCVLCVNCFNPKDHLGHHVYTTICTEFN |
| | | NGECDCGDKTAWNHTLFCKAEEGGGSGGGSGGGSGMHSKFSHAGRICGAKFKVGEPIYRC |
| | | KECSFDDTCVLCVNCFNPKDHLGHHVYTTICTEFNNGECDCGDKTAWNHTLFCKAEEG |
| PS1220 | 566 | MHSKFSHAGRICGAKFKVGEPIYRCKECSFDDTCVLCVNCFNPKDHLGHHVYTTICTEFN |
| | | NGECDCGDKTAWNHTLFCKAEEGSAGSAAGSGEFMHSKFSHAGRICGAKFKVGEPIYRCK |
| | | ECSFDDTCVLCVNCFNPKDHLGHHVYTTICTEFNNGECDCGDKTAWNHTLFCKAEEG |
| PS1221 | 567 | MHSKFSHAGRICGAKFKVGEPIYRCKECSFDDTCVLCVNCFNPKDHLGHHVYTTICTEFN |
| | | NGECDCGDKTAWNHTLFCKAEEGSAGSAAGSGEFGSAGSAAGSGEFGSAGSAAGSGEFMH |
| | | SKFSHAGRICGAKFKVGEPIYRCKECSFDDTCVLCVNCFNPKDHLGHHVYTTICTEFNNG |
| | | ECDCGDKTAWNHTLFCKAEEG |
| PS1222 | 568 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAFGRDRLLARSKGSMKASIEAEEG |
| | | GGSGGGSGGGSGMPTAASATESAIEDTPAPARPEVDGRTKPKRQPRYHVVLWDDDDHTYQ |
| | | YVVVMLRSLFGHPPSRGYRMAKEMDTQGRVIVLTTTREHAELKRDQIHAFGRDRLLARSK |
| | | GSMKASIEAEE |
| PS1223 | 569 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAFGRDRLLARSKGSMKASIEAEEG |
| | | SAGSAAGSGEFGSAGSAAGSGEFGSAGSAAGSGEFMPTAASATESAIEDTPAPARPEVDG |
| | | RTKPKRQPRYHVVLWDDDDHTYQYVVVMLRSLFGHPPSRGYRMAKEMDTQGRVIVLTTTR |
| | | EHAELKRDQIHAFGRDRLLARSKGSMKASIEAEE |
| PS1224 | 570 | MPTAASATESAIEDTPAPARPEVDGRTKPKRQPRYHVVLHDDDDHTYQYVVVMLRSLFGH |
| | | PPSRGYRMAKEMDTQGRVIVLTTTREHAELKRDQIHAFGRDRLLARSKGSMKASIEAEE |
| PS1225 | 571 | MPTAASATESAIEDTPAPARPEVDGRTKPKRQPRYHVVLWDDDDHTYQYVVVMLRSLFGI |
| | | PPSRGYRMAKEMDTQGRVIVLTTTREHAELKRDQIHAFGRDRLLARSKGSMKASIEAEE |
| PS1226 | 572 | MPTAASATESAIEDTPAPARPEVDGRTKPKRQPRYHVVLWDDDDHTAQYVVVMLRSLFGH |
| | | PPSRGYRMAKEMDTQGRVIVLTTTREHAELKRDQIHAFGRDRLLARSKGSMKASIEAEE |
| PS1227 | 573 | MPTAASATESAIEDTPAPARPEVDGRTKPKRQPRYHVVLWDDNDHTYQYVVVMLRSLFGH |
| | | PPSRGYRMAKEMDTQGRVIVLTTTREHAELKRDQIHAFGRDRLLARSKGSMKASIEAEE |
| PS1228 | 574 | MPTAASATESAIEDTPAPARPEVDGRTKPKRQPRYHVVLWNDDDHTYQYVVVMLRSLFGH |
| | | PPSRGYRMAKEMDTQGRVIVLTTTREHAELKRDQIHAFGRDRLLARSKGSMKASIEAEE |
| PS1229 | 575 | MPTAASATESAIEDTPAPARPEVDGRTKPKRQPRYHVVLWDDDDHTWQYVVVMLRSLFGH |
| | | PPSRGYRMAKEMDTQGRVIVLTTTREHAELKRDQIHAFGRDRLLARSKGSMKASIEAEE |
| PS1230 | 576 | MPTAASATESAIEDTPAPARPEVDGRTKPKRQPRYHVVLWDDDDHTYQYVVVMLRSLFGH |
| | | PPSRGYRMAKELDTQGRVIVLTTTREHAELKRDQIHAFGRDRLLARSKGSMKASIEAEE |
| PS1231 | 577 | MPTAASATESAIEDTPAPARPEVDGRTKPKRQPRYHVVLWDDDDHTYQYVVVMLRSLFGY |
| | | PPSRGYRMAKEMDTQGRVIVLTTTREHAELKRDQIHAFGRDRLLARSKGSMKASIEAEE |
| PS1232 | 578 | MPTAASATESAIEDTPAPARPEVDGRTKPKRQPRYHVVLWDDDDHTLQYVVVMLRSLFGH |
| | | PPSRGYRMAKEMDTQGRVIVLTTTREHAELKRDQIHAFGRDRLLARSKGSMKASIEAEE |
| PS1233 | 579 | MPTAASATESAIEDTPAPARPEVDGRTKPKRQPRYHVVLWDDDDHTIQYVVVMLRSLFGH |
| | | PPSRGYRMAKEMDTQGRVIVLTTTREHAELKRDQIHAFGRDRLLARSKGSMKASIEAEE |
| PS1234 | 580 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHASGRDRLLARSKGSMKASIEAEE |
| PS1235 | 581 | MPTAASATESAIEDTPAPARPEVDGRTKPKRQPRYHVVLWDDDDHTYQYVVVMLRSLFGH |
| | | PLSRGYRMAKEMDTQGRVIVLTTTREHAELKRDQIHAFGRDRLLARSKGSMKASIEAEE |
| PS1236 | 582 | MPTAASATESAIEDTPAPARPEVDGRTKPKRQPRYHVVLWDDDQHTYQYVVVMLRSLFGH |
| | | PPSRGYRMAKEMDTQGRVIVLTTTREHAELKRDQIHAFGRDRLLARSKGSMKASIEAEE |
| PS1237 | 583 | MPTAASATESAIEDTPAPARPEVDGRTKPKRQPRYHVVLWDDDDHTYQYVVVMLRSLFGH |
| | | PPSRGYRMAKEMDTQGRTIVLTTTREHAELKRDQIHAFGRDRLLARSKGSMKASIEAEE |
| PS1238 | 584 | MPTAASATESAIEDTPAPARPEVDGRTKPKRQPRYHVVLFDDDDHTYQYVVVMLRSLFGH |
| | | PPSRGYRMAKEMDTQGRVIVLTTTREHAELKRDQIHAFGRDRLLARSKGSMKASIEAEE |
| PS1239 | 585 | MPTAASATESAIEDTPAPARPEVDGRTKPKRQPRYHVVLWDDDDHTYQYVVVMLRSLFGH |
| | | PPSRGYRMTKEMDTQGRVIVLTTTREHAELKRDQIHAFGRDRLLARSKGSMKASIEAEE |
| PS1240 | 586 | MPTAASATESAIEDTPAPARPEVDGRTKPKRQPRYHVVLWDDDDHTYQYVVVMLRSLFGS |
| | | PPSRGYRMAKEMDTQGRLIVLTTTREHAELKRDQIHAFGRDRLLARSKGSMKASIEAEE |
| PS1241 | 587 | MPTAASATESAIEDTPAPARPEVDGRTKPKRQPRYHVVLWDDDDHTYQYVVVMLRSLFGV |
| | | PPSRGYRMAKELDTQGRVIVLTTTREHAELKRDQIHAFGRDRLLARSKGSMKASIEAEE |
| PS1242 | 588 | MPTAASATESAIEDTPAPARPEVDGRTKPKRQPRYHVVLWDDDDHTYQYVVVMLRSLFGL |
| | | PPSRGYRMAHEMDTQGRVIVLTTTREHAELKRDQIHAFGRDRLLARSKGSMKASIEAEE |
| PS1243 | 589 | MPTAASATESAIEDTPAPARPEVDGRTKPKRQPRYHVVLYDDDDHTDQYVVVMLRSLFGH |
| | | PPSRGYRLAEEMDTQGRVIVLTTTREHAELKRDQIHAFGRDRLLARSKGSMKASIEAEE |
| PS1244 | 590 | MPTAASATESAIEDTPAPARPEVDGRTKPKRQPRYHVVLLDDDSHTYQYVVVMLRSLFGV |
| | | PPSRGYRMAAEMDTQGRVIVLTTTREHAELKRDQIHAFGRDRLLARSKGSMKASIEAEE |
| PS1245 | 591 | MPTAASATESAIEDTPAPARPEVDGRTKPKRQPRYHVVLWDDHDHTYQYVVVMLRSLFGH |
| | | PPSRGYRMAKELHTQGRVIVLTTTREHAELKRDQIHAFGRDRLLARSKGSMKASIEAEE |
| PS1246 | 592 | MEGNGPAAVHYQPASPPRDACVYSSCYCEENVWKLCEYIKNHDQYPLEECYAVFISNERK |
| | | MIPIWKQQARPGDGPVIWDYHVVLLHVSSGGQSFIYDLDTVLPFPCLFDTYVEDAIKSDD |
| | | DIHPQFRRKFRVICADSYLKNFASDRSHMKDSSGNWREPPPPYPCIETGDSKMNLNDFIS |
| | | MDPKVGWGAVYTLSEFTHRFGSKN |
| PS1247 | 593 | MEGNGPAAVHYQPASPPRDACVYSSCYSEENVWKLCEYIKNHDQYPLEECYAVFISNERK |
| | | MIPIWKQQARPGDGPVIWDYHVVLLHVSSGGQSFIYDLDTVLPFPCLFDTYVEDAIKSDD |
| | | DIHPQFRRKFRVICADSYLKNFASDRSHMKDSSGNWREPPPPYPCIETGDSKMNLNDFIS |
| | | MDPKVGWGAVYTLSEFTHRFGSKN |
| PS1248 | 594 | MEGNGPAAVHYQPASPPRDACVYSSCYSEENVWKLCEYIKNHDQYPLEECYAVFISNERK |
| | | MIPIWKQQARPGDGPVIWDYQVVLLHVSSGGQSFIYDLDTVLPFPCLFDTYVEDAIKSDD |
| | | DIHPQFRRKFRVICADSYLKNFASDRSHMKDSSGNWREPPPPYPCIETGDSKMNLNDFIS |
| | | MDPKVGWGAVYTLSEFTHRFGSKN |
| PS1249 | 595 | MEGNGPAAVHYQPASPPRDACVYSSCYSEENVWKLCEYIKNHDQYPLEECYAVFISNERK |
| | | MIPIWKQQARPGDGPVIWDYQVVLLHVSSGGQSFIYDLDTVLPFPCLFDTYVEDAIKSDD |
| | | DIHPQFRRKFRVICADSYLKNFASDRSHEKDSSGNWREPPPPYPCIETGDSKMNLNDFIS |
| | | MDPKVGWGAVYTLSEFTHRFGSKN |
| PS1250 | 596 | MGPAATAPQYQPVCPTRDACVYNSCYSEENIWKLCEYIKTHNQYLLEECYAVFISNEKKM |
| | | VPIWKQQARPENGPVIWDYHVVLLHVSREGQSFIYDLDTILPFPCPFDIYIEDALKSDDD |
| | | IHLQFRRKFRVVRADSYLKHFASDRSHMKDSSGNWREPPPEYPCIETGDSKMNLNDFISM |
| | | DPAVGWGAVYTLPEFVHRFSSKTY |
| PS1251 | 597 | MGPAATAPQYQPVCPTRDACVYNSCYSEENIWKLCEYIKTHNQYLLEECYAVFISNEKKM |
| | | VPIWKQQARPENGPVIWDYQVVLLHVSREGQSFIYDLDTILPFPCPFDIYIEDALKSDDD |
| | | IHLQFRRKFRVVRADSYLKHFASDRSHMKDSSGNWREPPPEYPCIETGDSKMNLNDFISM |
| | | DPAVGWGAVYTLPEFVHRFSSKTY |
| PS1252 | 598 | MGPAATAPQYQPVCPTRDACVYNSCYSEENIWKLCEYIKTHNQYLLEECYAVFISNEKKM |
| | | VPIWKQQARPENGPVIWDYQVVLLHVSREGQSFIYDLDTILPFPCPFDIYIEDALKSDDD |
| | | IHLQFRRKFRVVRADSYLKHFASDRSHEKDSSGNWREPPPEYPCIETGDSKMNLNDFISM |
| | | DPAVGWGAVYTLPEFVHRFSSKTY |
| PS1253 | 599 | MVPAAAAARYQPASPPRDACVYNSCYSEENIWKLCEYIKNHDQYPLEECYAVFISNERKM |
| | | IPIWKQQARPGDGPVIWYYFFLLVRYHVKSIGFSFTFQAIPLVNTLEDILAQLFKFCIHM |
| | | HACVLWKFRVIRADSYLKNFASDRSHMKDSSGNWREPPPSYPCIETGDSKMNLNDFISMD |
| | | PEVGWGAVYSLSEFVHRFGSQNY |
| PS1254 | 600 | MVPAAAAARYQPASPPRDACVYNSCYSEENIWKLCEYIKNHDQYPLEECYAVFISNERKM |
| | | IPIWKQQARPGDGPVIWYYFFLLVRYHVKSIGFSFTFQAIPLVNTLEDILAQLFKFCIHM |
| | | HACVLWKFRVIRADSYLKNFASDRSHEKDSSGNWREPPPSYPCIETGDSKMNLNDFISMD |
| | | PEVGWGAVYSLSEFVHRFGSQNY |
| PS1255 | 601 | MAAGEPSPFLVRSDCLYTSCYSEENVWKLCEYIRDHRPCLLEQFSAVFISNENKMIPIWK |
| | | QKSAKGDGPVIWDYHVILLHESARDGNFVYDLDTILPFPSPCNTYIREALKCDSNIHCDF |
| | | RRKLRVVGAHEFLQTFASDRSHMRDSSSNWTKPPPPYPCIQTAESTMNLDDFISMNPEVG |
| | | WGTVYSLAAFIERFGDTTL |
| PS1256 | 602 | MAAGEPSPFLVRSDCLYTSCYSEENVWKLCEYIRDHRPCLLEQFSAVFISNENKMIPIWK |
| | | QKSAKGDGPVIWDYQVILLHESARDGNFVYDLDTILPFPSPCNTYIREALKCDSNIHCDF |
| | | RRKLRVVGAHEFLQTFASDRSHMRDSSSNWTKPPPPYPCIQTAESTMNLDDFISMNPEVG |
| | | WGTVYSLAAFIERFGDTTL |
| PS1257 | 603 | MAAGEPSPFLVRSDCLYTSCYSEENVWKLCEYIRDHRPCLLEQFSAVFISNENKMIPIWK |
| | | QKSAKGDGPVIWDYQVILLHESARDGNFVYDLDTILPFPSPCNTYIREALKCDSNIHCDF |
| | | RRKLRVVGAHEFLQTFASDRSHERDSSSNWTKPPPPYPCIQTAESTMNLDDFISMNPEVG |
| | | WGTVYSLAAFIERFGDTTL |
| PS1258 | 604 | MNGLSAQHERIAPARHECVYTSCYSEENVWKLCEHIKTSKRCPLGDVYAVFISNERKMVP |
| | | IWKQKSGRGEEPVIWDYHVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKT |
| PS1259 | 605 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKT |
| PS1260 | 606 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHEKDASGGWRMPPPPYPCIETAESRMNLDDFISMNP |
| | | SVGWGHVYTLEEFVQHFGKT |
| PS1261 | 607 | MESASSEYKVITPSGNQCVYTSCYSEENVWKLCEYIKNQRHCPLEEVYAVFISNERKKIP |
| | | IWKQKSSRGDEPVIWDYHVILLHASKQGPSFIYDLDTILPFPCSLDVYSMEAFQSDKHLK |
| | | PAYWRKLRVIPGDTYLKEFASDRSHMKDSDGNWRMPPPAYPCLETPESKMNLDDFICMDP |
| | | RVGYGEVYSLSDFVKHFGVK |
| PS1262 | | MESASSEYKVITPSGNQCVYTSCYSEENVWKLCEYIKNQRHCPLEEVYAVFISNERKKIP |
| | | IWKQKSSRGDEPVIWDYQVILLHASKQGPSFIYDLDTILPFPCSLDVYSMEAFQSDKHLK |
| | | PAYWRKLRVIPGDTYLKEFASDRSHMKDSDGNWRMPPPAYPCLETPESKMNLDDFICMDP |
| | | RVGYGEVYSLSDFVKHFGVK |
| PS1263 | 609 | MESASSEYKVITPSGNQCVYTSCYSEENVWKLCEYIKNQRHCPLEEVYAVFISNERKKIP |
| | | IWKQKSSRGDEPVIWDYQVILLHASKQGPSFIYDLDTILPFPCSLDVYSMEAFQSDKHLK |
| | | PAYWRKLRVIPGDTYLKEFASDRSHEKDSDGNWRMPPPAYPCLETPESKMNLDDFICMDP |
| | | RVGYGEVYSLSDFVKHFGVK |
| PS1264 | 610 | MEHVSSKYVNITPSRDECVYTSCYSEENVWKLCEHIKTQTQIHLDEVYAVFISNERKMIP |
| | | IWKQKSSRGDEPVVWDYHVVLLHQNQQGQSFIYDQDTVLPFSCPFHVYTTEAFHTDHGLK |
| | | PAFWRKLRVIPADTYLKNFASDRSHMKNADGTWRMPPPLYPCIETTDSKMNLDDFISMDS |
| | | KVGCGHVYSLSEFVKHFAEK |
| PS1265 | 611 | MEHVSSKYVNITPSRDECVYTSCYSEENVWKLCEHIKTQTQIHLDEVYAVFISNERKMIP |
| | | IWKQKSSRGDEPVVWDYQVVLLHQNQQGQSFIYDQDTVLPFSCPFHVYTTEAFHTDHGLK |
| | | PAFWRKLRVIPADTYLKNFASDRSHMKNADGTWRMPPPLYPCIETTDSKMNLDDFISMDS |
| | | KVGCGHVYSLSEFVKHFAEK |
| PS1266 | 612 | MEHVSSKYVNITPSRDECVYTSCYSEENVWKLCEHIKTQTQIHLDEVYAVFISNERKMIP |
| | | IWKQKSSRGDEPVVWDYQVVLLHQNQQGQSFIYDQDTVLPFSCPFHVYTTEAFHTDHGLK |
| | | PAFWRKLRVIPADTYLKNFASDRSHEKNADGTWRMPPPLYPCIETTDSKMNLDDFISMDS |
| | | KVGCGHVYSLSEFVKHFAEK |
| PS1267 | 613 | MGLENSLETLRFSISNLSMQTHAARMRTFMYWPSSVPVQPEQLASAGFYYLGRNDIVICF |
| | | CCDGGLHCWQSGDDPWVEHALFFPRCEFLIRMKGQEFVDEIQGRYPHLLEQLLSG |
| PS1268 | 614 | MHHHHHHHHHHDYDIPTTENLYFQGMHSKFSHAGRICGAKFKVGEPIYRCKECSFDDTCV |
| | | LCVNCFNPKDHLGHHVYTTICTEFNNGECDCGDKTAWNHTLFCKAEE |
| PS1270 | 615 | MHHHHHHHHHHDYDIPTTENLYFQGRFSISNLSMQTHAARMRTFMYWPSSVPVQPEQ |
| | | LASAGFYYVGRNDDVKCFCCDGGLRCWESGDDPWVEHAKWFPRCEFLIRMKGQEFVD |
| | | EIQGRY |
| PS1271 | 616 | MGDVQPETCRPSAASGNYFPQYPEYAIETARLRTFEAWPRNLKQKPHQLAEAGFFYTGVG |
| | | DRVRCFSCGGGLMDWNDNDEPWEQHARHLSQCRFVKLMKGQLYIDTVAAKPVLAEEKEES |
| | | TSIGGDGSAGSAAGSGEFMGDVQPETCRPSAASGNYFPQYPEYAIETARLRTFEAWPRNL |
| | | KQKPHQLAEAGFFYTGVGDRVRCFSCGGGLMDWNDNDEPWEQHARHLSQCRFVKLMKGQL |
| | | YIDTVAAKPVLAEEKEESTSIGGD |
| PS1272 | 617 | MGLENSLETLRFSISNLSMQTHAARMRTFMYWPSSVPVQPEQLASAGFYYVGRNDDVKCF |
| | | CCDGGLRCWESGDDPWVEHAKWFPRCEFLIRMKGQEFVDEIQGRYPHLLEQLLSGSAGSA |
| | | AGSGEFMGLENSLETLRFSISNLSMQTHAARMRTFMYWPSSVPVQPEQLASAGFYYVGRN |
| | | DDVKCFCCDGGLRCWESGDDPWVEHAKWFPRCEFLIRMKGQEFVDEIQGRYPHLLEQLLS |
| | | G |
| PS1273 | 618 | MGLENSLETLRFSISNLSMQTHAARMRTFMYWPSSVPVQPEQLASAGFYYVGRNDDVKCF |
| | | CCDGGLRCWESGDDPWVEHARHFPRCEFLIRMKGQEFVDEIQGRYPHLLEQLLSGSAGSA |
| | | AGSGEFMGLENSLETLRFSISNLSMQTHAARMRTFMYWPSSVPVQPEQLASAGFYYVGRN |
| | | DDVKCFCCDGGLRCWESGDDPWVEHARHFPRCEFLIRMKGQEFVDEIQGRYPHLLEQLLS |
| | | G |
| PS1274 | 619 | MGDVQPETCRPSAASGNYFPQYPEYAIETARLRTFEAWPRNLKQKPHQLAEAGFFYTG |
| | | VGDRVRCFSCGGGLMDWNDNDEPWEQHALWLSQCRFVKLMKGQLYIDTVAAKPVL |
| | | AEEKEESTSIGGDTGSAGSAAGSGEFMGDVQPETCRPSAASGNYFPQYPEYAIETARLR |
| | | TFEAWPRNLKQKPHQLAEAGFFYTGVGDRVRCFSCGGGLMDWNDNDEPWEQHAL |
| | | WLSQCRFVKLMKGQLYIDTVAAKPVLAEEKEESTSIGGDT |
| PS1275 | 620 | MGLENSLETLRFSISNLSMQTHAARMRTFMYWPSSVPVQPEQLASAGFYYVGRNDDVKCF |
| | | CCDGGLRCWESGDDPWVEHAKWFPRCEFLIRMKGQEFVDEIQGRYPHLLEQLLSGGGSGG |
| | | GSGGGSGMGLENSLETLRFSISNLSMQTHAARMRTFMYWPSSVPVQPEQLASAGFYYVGR |
| | | NDDVKCFCCDGGLRCWESGDDPWVEHAKWFPRCEFLIRMKGQEFVDEIQGRYPHLLEQLL |
| | | SG |
| PS1276 | 621 | MGLENSLETLRFSISNLSMQTHAARMRTFMYWPSSVPVQPEQLASAGFYYVGRNDDVKCF |
| | | CCDGGLRCWESGDDPWVEHAKWFPRCEFLIRMKGQEFVDEIQGRYPHLLEQLLSGSAGSA |
| | | AGSGEFGSAGSAAGSGEFGSAGSAAGSGEFMGLENSLETLRFSISNLSMQTHAARMRTFM |
| | | YWPSSVPVQPEQLASAGFYYVGRNDDVKCFCCDGGLRCWESGDDPWVEHAKWFPRCEFLI |
| | | RMKGQEFVDEIQGRYPHLLEQLLSG |
| PS1277 | 622 | MPTAASATESAIEDTPAPARPEVDGRTKPKRQPRYHVVLWDDEDHTYQYVVVMLRSLFGH |
| | | PPSRGYRMAKEMDTQGRVIVLTTTREHAELKRDQIHAFGRDRLLARSKGSMKASIEAEE |
| PS1278 | 623 | MPTAASATESAIEDTPAPARPEVDGRTKPKRQPRYHVVLWDDDDHTYQYVVVMLRSLFGH |
| | | PPSRGYRMAREMDTQGRVIVLTTTREHAELKRDQIHAFGRDRLLARSKGSMKASIEAEE |
| PS1279 | 624 | MPTAASATESAIEDTPAPARPEVDGRTKPKRQPRYHVVLWDDDDHTYQYVVVMLRSLFGH |
| | | PPSRGYRMAKEMTTQGRVIVLTTTREHAELKRDQIHAFGRDRLLARSKGSMKASIEAEE |
| PS1280 | 625 | MPTAASATESAIEDTPAPARPEVDGRTKPKRQPRYHVVLWDDQDHTYQYVVVMLRSLFGH |
| | | PPSRGYRMAKEMDTQGRVIVLTTTREHAELKRDQIHAFGRDRLLARSKGSMKASIEAEE |
| PS1281 | 626 | MPTAASATESAIEDTPAPARPEVDGRTKPKRQPRYHVVLWDDDEHTYQYVVVMLRSLFGH |
| | | PPSRGYRMAKEMDTQGRVIVLTTTREHAELKRDQIHAFGRDRLLARSKGSMKASIEAEE |
| PS1282 | 627 | MPTAASATESAIEDTPAPARPEVDGRTKPKRQPRYHVVLWDDDDHTMQYVVVMLRSLFGH |
| | | PPSRGYRMAKEMDTQGRVIVLTTTREHAELKRDQIHAFGRDRLLARSKGSMKASIEAEE |
| PS1283 | 628 | MPTAASATESAIEDTPAPARPEVDGRTKPKRQPRYHVVLWDDDDHTFQYVVVMLRSLFGH |
| | | PPSRGYRMAKEMDTQGRVIVLTTTREHAELKRDQIHAFGRDRLLARSKGSMKASIEAEE |
| PS1284 | 629 | MPTAASATESAIEDTPAPARPEVDGRTKPKRQPRYHVVLWDDDDHTVQYVVVMLRSLFGH |
| | | PPSRGYRMAKEMDTQGRVIVLTTTREHAELKRDQIHAFGRDRLLARSKGSMKASIEAEE |
| PS1285 | 630 | MPTAASATESAIEDTPAPARPEVDGRTKPKRQPRYHVVLYDDDTHTYQYVVVMLRSLFGH |
| | | PPSRGYRMAKEMDTQGRVIVLTTTREHAELKRDQIHAFGRDRLLARSKGSMKASIEAEE |
| PS1286 | 631 | MPTAASATESAIEDTPAPARPEVDGRTKPKRQPRYHVVLYDDEDHTYQYVVVMLRSLFGH |
| | | PPSRGYRMAKEMDTQGRVIVLTTTREHAELKRDQIHAFGRDRLLARSKGSMKASIEAEE |
| PS1287 | 632 | MPTAASATESAIEDTPAPARPEVDGRTKPKRQPRYHVVLYDDDDHTFQYVVVMLRSLFGH |
| | | PPSRGYRMAKEMDTQGRVIVLTTTREHAELKRDQIHAFGRDRLLARSKGSMKASIEAEE |
| PS1288 | 633 | MPTAASATESAIEDTPAPARPEVDGRTKPKRQPRYHVVLYDDDDHTYQYVVVMLRSLFGH |
| | | PPSRGYRMAREMDTQGRVIVLTTTREHAELKRDQIHAFGRDRLLARSKGSMKASIEAEE |
| PS1289 | 634 | MPTAASATESAIEDTPAPARPEVDGRTKPKRQPRYHVVLYDDDDHTYQYVVVMLRSLFGH |
| | | PPSRGYRMAKEMTTQGRVIVLTTTREHAELKRDQIHAFGRDRLLARSKGSMKASIEAEE |
| PS1290 | 635 | MPTAASATESAIEDTPAPARPEVDGRTKPKRQPRYHVVLWDDEDHTYQYVVVMLRSLFGH |
| | | PPSRGYRMAKEMTTQGRVIVLTTTREHAELKRDQIHAFGRDRLLARSKGSMKASIEAEE |
| PS1291 | 636 | MPTAASATESAIEDTPAPARPEVDGRTKPKRQPRYHVVLYDDEDHTFQYVVVMLRSLFGH |
| | | PPSRGYRMAKEMDTQGRVIVLTTTREHAELKRDQIHAFGRDRLLARSKGSMKASIEAEE |
| PS1292 | 637 | MPTAASATESAIEDTPAPARPEVDGRTKPKRQPRYHVVLYDDDDHTYQYVVVMLRSLFGH |
| | | PPSRGYRMAREMTTQGRVIVLTTTREHAELKRDQIHAFGRDRLLARSKGSMKASIEAEE |
| PS1293 | 638 | MPTAASATESAIEDTPAPARPEVDGRTKPKRQPRYHVVLWDDEDHTYQYVVVMLRSLFGH |
| | | PPSRGYRMAREMTTQGRVIVLTTTREHAELKRDQIHAFGRDRLLARSKGSMKASIEAEE |
| PS1294 | 639 | MPTAASATESAIEDTPAPARPEVDGRTKPKRQPRYHVVLYDDEDHTFQYVVVMLRSLFGH |
| | | PPSRGYRMAKEMTTQGRVIVLTTTREHAELKRDQIHAFGRDRLLARSKGSMKASIEAEE |
| PS1295 | 640 | MPTAASATESAIEDTPAPARPEVDGRTKPKRQPRYHVVLWDDEDHTFQYVVVMLRSLFGH |
| | | PPSRGYRMAREMTTQGRVIVLTTTREHAELKRDQIHAFGRDRLLARSKGSMKASIEAEE |
| PS1296 | 641 | MPTAASATESAIEDTPAPARPEVDGRTKPKRQPRYHVVLYDDEDHTFQYVVVMLRSLFGH |
| | | PPSRGYRMAREMTTQGRVIVLTTTREHAELKRDQIHAFGRDRLLARSKGSMKASIEAEE |
| PS1297 | 642 | MPTAASATESAIEDTPAPARPEVDGRTKPKRQPRYHVVLWSDEDHTHQYVVVMLRSLFGH |
| | | PPSRGYRMAKEIDTQGRVIVLTTTREHAELKRDQIHAFGRDRLLARSKGSMKASIEAEE |
| PS1298 | 643 | MPTAASATESAIEDTPAPARPEVDGRTKPKRQPRYHVVIWEDDDHTYQYWVVMLRSLFGH |
| | | PPSRGYRMAKEAHTQGRVIVLTTTREHAELKRDQIHAFGRDRLLARSKGSLKASIEAEE |
| PS1299 | 644 | MPTAASATESAIEDTPAPARPEVDGRTKPKRQPRYHVVIWSDDDHTHDYVVVMLRSLFGH |
| | | PPSRGYRMTKELHTQGRVIVLTTTREHAELKRDQIHAFGRDRLLARSKGSMKASIEAEE |
| PS1300 | 645 | MPTAASATESAIEDTPAPARPEVDGRTKPKRQPRYHVVMWDDQDNTDQYWVVMLRSLFGH |
| | | PPSRGYRMSEELHTQGRVIVLTTTREHAELKRDQIHAFGRDRLLARSKGSVKASIEAEE |
| PS1301 | 646 | MPTAASATESAIEDTPAPARPEVDGRTKPKRQPRYHVVVWDDEDHTHQYWVVMLRSLFGH |
| | | PPSRGYRMAKEAHTQGRVIVLTTTREHAELKRDQIHAFGRDRLLARSKGSLKASIEAEE |
| PS1302 | 647 | MPTAASATESAIEDTPAPARPEVDGRTKPKRQPRYHVVLWDDDDHTSQYVVVMLHSLFGH |
| | | PPSRGYRLAKEMDTQGRVIVLTTTREHAELKRDQIHAFGRDRLLARSKGSMKASIEAEE |
| PS1303 | 648 | MPTAASATESAIEDTPAPARPEVDGRTKPKRQPRYHVVLWSDEDHTHQYIVVMLRSLFGH |
| | | PPSRGYRMAKEIDTQGRVIVLTTTREHAELKRDQIHAFGRDRLLARSKGSMKASIEAEE |
| PS1304 | 649 | MPTAASATESAIEDTPAPARPEVDGRTKPKRQPRYHVVLWDDDDHTKQYIVVMLRSLFGH |
| | | PPSRGYRMAKEMHTQGRVIVLTTTREHAELKRDQIHAFGRDRLLARSKGSVKASIEAEE |
| PS1305 | 650 | MPTAASATESAIEDTPAPARPEVDGRTKPKRQPRYHVVMWEDEDHTFQYVVVMLRSLFGH |
| | | PPSRGYRMVKEMHTQGRVIVLTTTREHAELKRDQIHAFGRDRLLARSKGSMKASIEAEE |
| PS1306 | 651 | MASDTPESLMALCTDFCLRNLDGTLGYLLDKETLRLHPDIFLPSEICDRLVNEYVELVNA |
| | | ACNFEPHESFFSLFSDPRSTRLTRIHLREDLVQDQDLEAIRKQDLVELYLTNCEKLSAKS |
| | | LQTLRSFSHTLVSLSLFGCTNIFYEEENPGGCEDEYLVNPTCQVLVKDFTFEGFSRLRFL |
| | | NLGRMIDWVPVESLLRPLNSLAALDLSGIQTSDAAFLTQWKDSLVSLVLYNMDLSDDHIR |
| | | VIVQLHKLRHLDISRDRLSSYYKFKLTREVLSLFVQKLGNLMSLDISGHMILENCSISKM |
| | | EEEAGQTSIEPSKSSIIPFRALKRPLQFLGLFENSLCRLTHIPAYKVSGDKNEEQVLNAI |
| | | EAYTEHRPEITSRAINLLFDIARIERCNQLLRALKLVITALKCHKYDRNIQVTGSAALFY |
| | | LTNSEYRSEQSVKLRRQVIQVVLNGMESYQEVTVQRNCCLTLCNFSIPEELEFQYRRVNE |
| | | LLLSILNPTRQDESIQRIAVHLCNALVCQVDNDHKEAVGKMGFVVTMLKLIQKKLLDKTC |
| | | DQVMEFSWSALWNITDETPDNCEMFLNFNGMKLFLDCLKEFPEKQELHRNMLGLLGNVAE |
| | | VKELRPQLMTSQFISVFSNLLESKADGIEVSYNACGVLSHIMFDGPEAWGVCEPQREEVE |
| | | ERMWAAIQSWDINSRRNINYRSFEPILRLLPQGISPVSQHWATWALYNLVSVYPDKYCPL |
| | | LIKEGGMPLLRDIIKMATARQETKEMARKVIEHCSNFKEENMDTSR |
| PS1307 | 652 | MLTNSEYRSEQSVKLRRQVIQVVLNGMESYQEVTVQRNCCLTLCNFSIPEELEFQYRRVN |
| | | ELLLSILNPTRQDESIQRIAVHLCNALVCQVDNDHKEAVGKMGFVVTMLKLIQKKLLDKT |
| | | CDQVMEFSWSALWNITDETPDNCEMFLNFNGMKLFLDCLKEFPEKQELHRNMLGLLGNVA |
| | | EVKELRPQLMTSQFISVFSNLLESKADGIEVSYNACGVLSHIMFDGPEAWGVCEPQREEV |
| | | EERMWAAIQSWDINSRRNINYRSFEPILRLLPQGISPVSQHWATWALYNLVSVYPDKYCP |
| | | LLIKEGGMPLLRDIIKMATARQETKEMARKVIEHCSNFKEENMDTSR |
| PS1308 | 653 | MLTNSEYRMEQSIKLRRQVIQVVLNGMESYQEVTVQRNCCLTLCNFSIPEELEFQYRRVN |
| | | ELLLSILNQSRQDESIQRIAVHLCNALVCQVDNDHKEAVGKMGFVMTMLKLIQKKLADKT |
| | | CDQVMEFSWSALWNITDETPDNCEMFLNYSGMKLFLECLKEFPEKQELHRNMLGLLGNVA |
| | | EVRELRPQLMTSQFISVFSNLLESKADGIEVSYNACGVLSHIMFDGPEAWGICEPHREEV |
| | | VKRMWAAIQSWDINSRRNINYRSFEPILRLLPQGISPVSQHWATWALYNLVSVYPDKYCP |
| | | LLIKEGGIPLLKDMIKMASARQETKEMAWKVIEHCSNFKEENMDTSR |
| PS1309 | 654 | MTGSAALFYLTNTEYRGEQSVRLRRQVIQVVLNGMEHYQEVTVQRNCCLTLCNFSIPEEL |
| | | EFQYRRVNLLLLKILEPLRQDESIQRIAVHLCNALVCQVDNDHKEAVGKMGFVKTMLNLI |
| | | QKKLQDRMCDQVMEFSWSALWNITDETPDNCQMFLECNGMNLFLECLKEFPDKQELHRNM |
| | | LGLLGNVAEVKALRPQLLTRQFITVFSDLLDSKADGIEVSYNACGVLSHIMFDGPGVWSM |
| | | EEPSRTHVMDKMWTAIQSWDVSSRRNINYRSFEPILRLLPQSGAPVSQHWATWALYNLVS |
| | | VYPSKYCPLLIKEGGVSLLQAVLELQTSHVETKDMARKVMEQCESFKEDPMDTSR |
| PS1310 | 655 | MPEDQAGAAMEEASPYSLLDICLNFLTTHLEKFCSARQDGTLCLQEPGVFPQEVADRLLR |
| | | TMAFHGLLNDGTVGIFRGNQMRLKRACIRKAKISAVAFRKAFCHHKLVELDATGVNADIT |
| | | ITDIISGLGSNKWIQQNLQCLVLNSLTLSLEDPYERCFSRLSGLRALSITNVLFYNEDLA |
| | | EVASLPRLESLDISNTSITDITALLACKDRLKSLTMHHLKCLKMTTTQILDVVRELKHLN |
| | | HLDISDDKQFTSDIALRLLEQKDILPNLVSLDVSGRKHVTDKAVEAFIQQRPSMQFVGLL |
| | | ATDAGYSEFLTGEGHLKVSGEANETQIAEALKRYSERAFFVREALFHLFSLTHVMEKTKP |
| | | EILKLVVTGMRNHPMNLPVQLAASACVFNLTKQDLAAGMPVRLLADVTHLLLKAMEHFPN |
| | | HQQLQKNCLLSLCSDRILQDVPFNRFEAAKLVMQWLCNHEDQNMQRMAVAIISILAAKLS |
| | | TEQTAQLGTELFIVRQLLQIVKQKTNQNSVDTTLKFTLSALWNLTDESPTTCRHFIENQG |
| | | LELFMRVLESFPTESSIQQKVLGLLNNIAEVQELHSELMWKDFIDHISSLLHSVEVEVSY |
| | | FAAGIIAHLISRGEQAWTLSRSQRNSLLDDLHSAILKWPTPECEMVAYRSFNPFFPLLGC |
| | | FTTPGVQLWAVWAMQHVCSKNPSRYCSMLIEEGGLQHLYNIKDHEHTDPHVQQIAVAILD |
| | | SLEKHIVRHGRPPPCKKQPQARLN |
| PS1311 | 656 | MVFNLTKQDLAAGMPVRLLADVTHLLLKAMEHFPNHQQLQKNCLLSLCSDRILQDVPFNR |
| | | FEAAKLVMQWLCNHEDQNMQRMAVAIISILAAKLSTEQTAQLGTELFIVRQLLQIVKQKT |
| | | NQNSVDTTLKFTLSALWNLTDESPTTCRHFIENQGLELFMRVLESFPTESSIQQKVLGLL |
| | | NNIAEVQELHSELMWKDFIDHISSLLHSVEVEVSYFAAGIIAHLISRGEQAWTLSRSQRN |
| | | SLLDDLHSAILKWPTPECEMVAYRSFNPFFPLLGCFTTPGVQLWAVWAMQHVCSKNPSRY |
| | | CSMLIEEGGLQHLYNIKDHEHTDPHVQQIAVAILDSLEKHIVRHGRPPPCKKQPQARLN |
| PS1312 | 657 | MPEMLKLVVIGMRNHPTNLPVQLAASACVFNLTKQDLAAGMPVKLLADVTHLLLEAMKHF |
| | | PNHQQLQKNCLLSLCSDRILQDVPFNRFDAAKLVMQWLCNHEDQNMQRMAVAIISILAAK |
| | | LSTEQTAQLGAELFIVRQLLQIVRQKTSQNMVDTTLKFTLSALWNLTDESPTTCRHFIEN |
| | | QGLELFMKVLETFPSESSIQQKVLGLLNNIAEVKELHSELMCKDFIDQISKLLHSVEVEV |
| | | SYFAAGIIAHLVSRGEESWTLSSSLRETLLEQLHSAILSWPTPECEMVAYRSFNPFFPLL |
| | | ACFRTPGVQLWAVWAMQHVCSKNPVRYCSMLIEEGGLVRLHRIRDHMCADPDVLRITIAI |
| | | LDNLDRHLRKHGNPPCPKPPFAK |
| PS1313 | 658 | MLTHAIEKPRPDILKLVALGMKNHPTTLNVQLAASACVFNLTKQELAFGIPVRLLGNVTQ |
| | | QLLEAMKTFPNHQQLQKNCLLSLCSDRILQEVPFNRFEAAKLVMQWLCNHEDQNMQRMAV |
| | | AIISILAAKLSTEQTAQLGAELFIVKQLLHIVRQKTCQSTVDATLKFTLSALWNLTDESP |
| | | TTCRHFIENQGLELFIKVLESFPSESSIQQKVLGLLNNIAEVSELHGELMVQSFLDHIRT |
| | | LLHSPEVEVSYFAAGILAHLTSRGEKVWTLELTLRNTLLQQLHSAILKWPTPECEMVAYR |
| | | SFNPFFPLLECFQTPGVQLWAAWAMQHVCSKNAGRYCSMLLEEGGLQHLEAITSHPKTHS |
| | | DVRRLTESILDGLQRHRARTGYTAIPKTQAHREKCNP |
| PS1314 | 659 | MEGNGPAAVHYQPASPPRDACVYSSCYSEENVWKLCEYIKNHDQYPLEECYAVFISNERK |
| | | MIPIWKQQARPGDGPVIWDYQVVLLHVSSGGQSFIYDLDTVLPFPCLFDTYVEDAIKSDD |
| | | DIHPQFRRKFRVICADSYLKNFASDRSHMKDSSGNWREPPPPYPCIETGDSKMNLNDFIS |
| | | MDPKVGWGAVYTLSEFTHRFGSKNGSAGSAAGSGEFMEGNGPAAVHYQPASPPRDACVYS |
| | | SCYSEENVWKLCEYIKNHDQYPLEECYAVFISNERKMIPIWKQQARPGDGPVIWDYQVVL |
| | | LHVSSGGQSFIYDLDTVLPFPCLFDTYVEDAIKSDDDIHPQFRRKFRVICADSYLKNFAS |
| | | DRSHMKDSSGNWREPPPPYPCIETGDSKMNLNDFISMDPKVGWGAVYTLSEFTHRFGSKN |
| PS1315 | 660 | MNGLSAQHERIAPARHECVYTSCYSEENVWKLCEHIKTSKRCPLGDVYAVFISNERKMVP |
| | | IWKQKSGRGEEPVIWDYHVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKTGSAGSAAGSGEFMNGLSAQHERIAPARHECVYTSCYSEEN |
| | | VWKLCEHIKTSKRCPLGDVYAVFISNERKMVPIWKQKSGRGEEPVIWDYHVILLHDCHKE |
| | | QTFIYDLDTTLPFPCPFDTYVKEAFKSDNYINPAFWRKLRVVPADVFLQNFASDRSHMKD |
| | | ASGGWRMPPPPYPCIETAESRMNLDDFISMNPSVGWGHVYTLEEFVQHFGKT |
| PS1316 | 661 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKTGGGSGGGSGGGSGMNGLSAQHERIAPARHECVYTSCYSEE |
| | | NVWKLCEHIKTSKRCPLGDVYAVFISNERKMVPIWKQKSGRGEEPVIWDYQVILLHDCHK |
| | | EQTFIYDLDTTLPFPCPFDTYVKEAFKSDNYINPAFWRKLRVVPADVFLQNFASDRSHMK |
| | | DASGGWRMPPPPYPCIETAESRMNLDDFISMNPSVGWGHVYTLEEFVQHFGKT |
| PS1317 | 662 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKTGSAGSAAGSGEFMNGLSAQHERIAPARHECVYTSCYSEEN |
| | | VWKLCEHIKTSKRCPLGDVYAVFISNERKMVPIWKQKSGRGEEPVIWDYQVILLHDCHKE |
| | | QTFIYDLDTTLPFPCPFDTYVKEAFKSDNYINPAFWRKLRVVPADVFLQNFASDRSHMKD |
| | | ASGGWRMPPPPYPCIETAESRMNLDDFISMNPSVGWGHVYTLEEFVQHFGKT |
| PS1318 | 663 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKTGSAGSAAGSGEFGSAGSAAGSGEFGSAGSAAGSGEFMNGL |
| | | SAQHERIAPARHECVYTSCYSEENVWKLCEHIKTSKRCPLGDVYAVFISNERKMVPIWKQ |
| | | KSGRGEEPVIWDYQVILLHDCHKEQTFIYDLDTTLPFPCPFDTYVKEAFKSDNYINPAFW |
| | | RKLRVVPADVFLQNFASDRSHMKDASGGWRMPPPPYPCIETAESRMNLDDFISMNPSVGW |
| | | GHVYTLEEFVQHFGKT |
| PS1321 | 664 | MPTAASATESAIEDTPAPARPEVDGRTKPKRQPRYHVVLWDDDDHTYQYVVAMLRSLFGH |
| | | PPSRGYRMAKEMDTQGRVIVLTTTREHAELKRDQIHAFGRDRLLARSKGSMKASIEAEE |
| PS1322 | 665 | MPTAASATESAIEDTPAPARPEVDGRTKPKRQPRYHVVLWDDDDHTYQYVVMMLRSLFGH |
| | | PPSRGYRMAKEMDTQGRVIVLTTTREHAELKRDQIHAFGRDRLLARSKGSMKASIEAEE |
| PS1323 | 666 | MPTAASATESAIEDTPAPARTEVDGRTKPKRQPRYHVVLWDDDDHTYQYVVVMLRSLFGH |
| | | PPSRGYRMAKEMDTQGRVIVLTTTREHAELKRDQIHAFGRDRLLARSKGSMKASIEAEE |
| PS1324 | 667 | MPTAASATESAIEDTPAPARPEVDGRTKPKRQPRYHVVLWDDDDHTYQYVVAMLRSLFGI |
| | | PPSRGYRMAKEMDTQGRVIVLTTTREHAELKRDQIHAFGRDRLLARSKGSMKASIEAEE |
| PS1325 | 668 | MPTAASATESAIEDTPAPARPEVDGRTKPKRQPRYHVVLWDDDDHTYQYVVMMLRSLFGI |
| | | PPSRGYRMAKEMDTQGRVIVLTTTREHAELKRDQIHAFGRDRLLARSKGSMKASIEAEE |
| PS1326 | 669 | MPTAASATESAIEDTPAPARPEVDGRTKPKRQPRYHVVLWDDDDHTYQYVVMMLRSLFGY |
| | | PPSRGYRMAKEMDTQGRVIVLTTTREHAELKRDQIHAFGRDRLLARSKGSMKASIEAEE |
| PS1327 | 670 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAWGRDRLLARSKGSMKASIEAEE |
| PS1328 | 671 | MPTAASATESAIEDTPAPARPEVDGRTKPKRQPRYHVVLWDDDDHTYQYVVVMLRSLFGH |
| | | PKSRGYRMAKEMDTQGRVIVLTTTREHAELKRDQIHAFGRDRLLARSKGSMKASIEAEE |
| PS1329 | 672 | MPTAASATESAIEDTPAPARPEVDGRTKPKRQPRYHVVLWDDDDHTYQYVVVMLQSLFGH |
| | | PPSRGYRMAKEMDTQGRVIVLTTTREHAELKRDQIHAFGRDRLLARSKGSMKASIEAEE |
| PS1330 | 673 | MPTAASATESAIEDTPAPARPEVDGRTKPKRQPRYHVVLWDDDDHTYQYVVVMLKSLFGH |
| | | PPSRGYRMAKEMDTQGRVIVLTTTREHAELKRDQIHAFGRDRLLARSKGSMKASIEAEE |
| PS1331 | 674 | MPTAASATESAIEDTPAPARPEVDGRTKPKRQPRYHVVLWDDDDHTYQYVVVMLASLFGH |
| | | PPSRGYRMAKEMDTQGRVIVLTTTREHAELKRDQIHAFGRDRLLARSKGSMKASIEAEE |
| PS1332 | 675 | MPTAASATESAIEDTPAPARPEVDGRTKPKRQPRYHVVLWDDDDHTYQYVVVMLSSLFGH |
| | | PPSRGYRMAKEMDTQGRVIVLTTTREHAELKRDQIHAFGRDRLLARSKGSMKASIEAEE |
| PS1333 | 676 | MPTAASATESAIEDTPAPARPEVDGRTKPKRQPRYHVVLWDDDDHTYQYVVVMLRSLFGH |
| | | PPQRGYRMAKEMDTQGRVIVLTTTREHAELKRDQIHAFGRDRLLARSKGSMKASIEAEE |
| PS1334 | 677 | MPTAASATESAIEDTPAPARPEVDGRTKPKRQPRYHVVLWDDDDHTYQYVVHMLRSLFGH |
| | | PPSRGYRMAKEMDTQGRVIVLTTTREHAELKRDQIHAFGRDRLLARSKGSMKASIEAEE |
| PS1335 | 678 | MPTAASATESAIEDTPAPARPEVDGRTKPKRQPRYHVVLWDDDDHTYQYVVVMLRSVFGH |
| | | PPSRGYRMAKEMDTQGRVIVLTTTREHAELKRDQIHAFGRDRLLARSKGSMKASIEAEE |
| PS1336 | 679 | MPTAASATESAIEDTPAPARPEVDGKTKPKRQPRYHVVLWDDDDHTYQYVVVMLRSLFGH |
| | | PPSRGYRMAKEMDTQGRVIVLTTTREHAELKRDQIHAFGRDRLLARSKGSMKASIEAEE |
| PS1337 | 680 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAYGRDRLLARSKGSMKASIEAEE |
| PS1338 | 681 | MPTAASATESAIEDTPAPARSEVDGYTVPKRQQRYHVVLWDDDDHTYQYVVYMLRSVFGH |
| | | PPSRGYRMAKEMDTQGRVIVLTTTREHAELKRDQIHAFGRDRLLARSKGSMKASIEAEE |
| PS1339 | 682 | MPTAASATESAIEDTPAPARPEVDGRTKPKRQPRYHVVLWDDDDHTYQYVVYMLRSVFGH |
| | | PPSRGYRMAKEMDTQGRVIVLTTTREHAELKRDQIHAFGRDRLLARSKGSMKASIEAEE |
| PS1340 | 683 | MPTAASATESAIEDTPAPARPEVDGRTKPKHQPRYHVVLWDDDDHTLQYVVVMLRSLFGH |
| | | PPSRGYRMAQEMETQGRVIVLTTTREHAELKRDQIHAFGRDRLLARSKGSMKASIEAEE |
| PS1341 | 684 | MPTAASATESAIEDTPAPARPEVDGRTKPKRQPRYHVVLWDDDDHTYQYVVEMLRHLFGH |
| | | PPSRGYRMAKEMDTQGRVIVLTTTREHAELKRDQIHAFGRDRLLARSKGSMKASIEAEE |
| PS1342 | 685 | MPTAASATESAIEDTPAPARPEVDGRTKPKRQPRYHVVLWDDDDHTYQYVVSMLRSVFGH |
| | | PPSRGYRMAKEMDTQGRVIVLTTTREHAELKRDQIRARGRDPLLARSKGSMKASIEAEE |
| PS1343 | 686 | MPTAASATESAIEDTPAPARPEVDGRTKPKRQPRYHVVLWDDDDHTYQYVVAMLRSIFGH |
| | | PPSRGYRMAKEMDTQGRVIVLTTTREHAELKRDQIHAFGRDRLLARSKGSMSASIEAEE |
| PS1344 | 687 | MPTAASATESAIEDTPAPARPEVDGRTKPKRQPRYHVVLWDDDDHTYQYVVVMLRSLFGH |
| | | PPSRGYRMAKELETQGRLIVLTTTREHAELKRDQIHAFGRDRLLARSKGSMKASIEAEE |
| PS1345 | 688 | MPTAASATESAIEDTPAPARPEVDGRTKPKHQPRYHVVLWDDDDHTDQYVVVMLRSLFGH |
| | | PPSRGYRMAKEMDTQGRVIVLTTTREHAELKRDQIHAFGRDRLLARSKGSMSASIEAEE |
| PS1346 | 689 | MPTAASATESAIEDTPAPARPEVDGRTKPKRQPRYHVVLWDDDDHTDQYVVVMLRSLFGH |
| | | PPSRGYRMAKEMDTQGRVIVLTTTREHAELKRDQIHAFGRDRLLARSKGSMSASIEAEE |
| PS1347 | 690 | MPTAASATESAIEDTPAPARPEVDGYTVPKRQPRYHVVLWDDDDHTYQYVVVMLRSLFGH |
| | | PPSRGYRMAKEMDTQGRVIVLTTTREHAELKRDQIHAYGRDRLLARSKGSMKASIEAEE |
| PS1348 | 691 | MPTAASATESAIEDTPAPARPEVDGRTKPKRQPRYHVVLWDDDDHTYQYVVTMLRSVFGH |
| | | PPSRGYRMAKEMDTQGRVIVLTTTREHAELKRDQIHAFGRDRLLARSKGSMKASIEAEE |
| PS1349 | 692 | MPTAASATESAIEDTPAPARSEVDGYTVPKRQPRYHVVLWDDDDHTYQYVVIMLRSLFGH |
| | | PPSRGYRMAKEMDTQGRVTVLTTTREHAELKRDQIHAFGRDRLLARSKGSMKASIEAEE |
| PS1350 | 693 | MPTAASATESAIEDTPAPARPEVDGRTKPKRQPRYHVVLWDDDDHTYQYVVIMLRSLFGH |
| | | PPSRGYRMAKEMDTQGRVTVLTTTREHAELKRDQIHAFGRDRLLARSKGSMKASIEAEE |
| PS1351 | 694 | MHSKFSHAGRICGAKFKVGERIYRCKECSFDDTCVLCVNCFNPKDHLGHHVYTTICTEFN |
| | | NGECDCGDKTAWNHTLFCKAEEG |
| PS1352 | 695 | MHSKFSHAGRICGAKFKVGEPIYRCRECSFDDTCVLCVNCFNPKDHLGHHVYTTICTEFN |
| | | NGECDCGDKTAWNHTLFCKAEEG |
| PS1353 | 696 | MHSKFSHAGRICGAKFKVGEPIYRCKLCSFDDTCVLCVNCFNPKDHLGHHVYTTICTEFN |
| | | NGECDCGDKTAWNHTLFCKAEEG |
| PS1354 | 697 | MHSKFSHAGRICGAKFKVGEPIYRCKECSFDATCVLCVNCFNPKDHLGHHVYTTICTEFN |
| | | NGECDCGDKTAWNHTLFCKAEEG |
| PS1355 | 698 | MHSKFSHAGRICGAKFKVGEPIYRCKECSFDCTCVLCVNCFNPKDHLGHHVYTTICTEFN |
| | | NGECDCGDKTAWNHTLFCKAEEG |
| PS1356 | 699 | MHSKFSHAGRICGAKFKVGEPIYRCKECSFDGTCVLCVNCFNPKDHLGHHVYTTICTEFN |
| | | NGECDCGDKTAWNHTLFCKAEEG |
| PS1357 | 700 | MHSKFSHAGRICGAKFKVGEPIYRCKECSFDHTCVLCVNCFNPKDHLGHHVYTTICTEFN |
| | | NGECDCGDKTAWNHTLFCKAEEG |
| PS1358 | 701 | MHSKFSHAGRICGAKFKVGEPIYRCKECSFDKTCVLCVNCFNPKDHLGHHVYTTICTEFN |
| | | NGECDCGDKTAWNHTLFCKAEEG |
| PS1359 | 702 | MHSKFSHAGRICGAKFKVGEPIYRCKECSFDPTCVLCVNCFNPKDHLGHHVYTTICTEFN |
| | | NGECDCGDKTAWNHTLFCKAEEG |
| PS1360 | 703 | MHSKFSHAGRICGAKFKVGEPIYRCKECSFDQTCVLCVNCFNPKDHLGHHVYTTICTEFN |
| | | NGECDCGDKTAWNHTLFCKAEEG |
| PS1361 | 704 | MHSKFSHAGRICGAKFKVGEPIYRCKECSFDRTCVLCVNCFNPKDHLGHHVYTTICTEFN |
| | | NGECDCGDKTAWNHTLFCKAEEG |
| PS1362 | 705 | MHSKFSHAGRICGAKFKVGEPIYRCKECSFDSTCVLCVNCFNPKDHLGHHVYTTICTEFN |
| | | NGECDCGDKTAWNHTLFCKAEEG |
| PS1363 | 706 | MHSKFSHAGRICGAKFKVGEPIYRCKECSFDVTCVLCVNCFNPKDHLGHHVYTTICTEFN |
| | | NGECDCGDKTAWNHTLFCKAEEG |
| PS1364 | 707 | MHSKFSHAGRICGAKFKVGEPIYRCKECSFDDTCVLCVNCFNPKDHIGHHVYTTICTEFN |
| | | NGECDCGDKTAWNHTLFCKAEEG |
| PS1365 | 708 | MHSKFSHAGRICGAKFKVGEPIYRCKECSFDDTCVLCVNCFNPKDHLGHHVYTTIRTEFN |
| | | NGECDCGDKTAWNHTLFCKAEEG |
| PS1366 | 709 | MHSKFSHAGRICGAKFKVGEPIYRCKECSFDDTCVLCVNCFNPKDHLGHHVYTTICTCFN |
| | | NGECDCGDKTAWNHTLFCKAEEG |
| PS1367 | 710 | MHSKFSHAGRICGAKFKVGEPIYRCKECSFDDTCVLCVNCFNPKDHLGHHVYTTICTDFN |
| | | NGECDCGDKTAWNHTLFCKAEEG |
| PS1368 | 711 | MHSKFSHAGRICGAKFKVGEPIYRCKECSFDDTCVLCVNCFNPKDHLGHHVYTTICTIFN |
| | | NGECDCGDKTAWNHTLFCKAEEG |
| PS1369 | 712 | MHSKFSHAGRICGAKFKVGEPIYRCKECSFDDTCVLCVNCFNPKDHLGHHVYTTICTNFN |
| | | NGECDCGDKTAWNHTLFCKAEEG |
| PS1370 | 713 | MHSKFSHAGRICGAKFKVGEPIYRCKECSFDDTCVLCVNCFNPKDHLGHHVYTTICTQFN |
| | | NGECDCGDKTAWNHTLFCKAEEG |
| PS1371 | 714 | MHSKFSHAGRICGAKFKVGEPIYRCKECSFDDTCVLCVNCFNPKDHLGHHVYTTICTSFN |
| | | NGECDCGDKTAWNHTLFCKAEEG |
| PS1372 | 715 | MHSKFSHAGRICGAKFKVGEPIYRCKECSFDDTCVLCVNCFNPKDHLGHHVYTTICTWFN |
| | | NGECDCGDKTAWNHTLFCKAEEG |
| PS1373 | 716 | MHSKFSHAGRICGAKFKVGEPIYRCKECSFDDTCVLCVNCFNPKDHLGHHVYTTICTEKN |
| | | NGECDCGDKTAWNHTLFCKAEEG |
| PS1374 | 717 | MHSKFSHAGRICGAKFKVGEPIYRCKECSFDKTCVLCVNCFNPKDHIGHHVYTTICTEFN |
| | | NGECDCGDKTAWNHTLFCKAEEG |
| PS1375 | 718 | MHSKFSHAGRICGAKFKVGEPIYRCKECSFDVTCVLCVNCFNPKDHLGHHVYTTIRTCKN |
| | | NGECDCGDKTAWNHTLFCKAEEG |
| PS1376 | 719 | MHSKFSHAGRICGAKFKVGERIYRCKECSFDDTCVLCVNCFNPKDHLGHHVYTTIRTEFN |
| | | NGECDCGDKTAWNHTLFCKAEEG |
| PS1377 | 720 | MHSKFSHAGRICGAKFKVGERIYRCKECSFDDTCVLCVNCFNPKDHLGHHVYTTIRTEKN |
| | | NGECDCGDKTAWNHTLFCKAEEG |
| PS1378 | 721 | MHSKFSHAGRICGAKFKVGEPIYRCRECSFDDTCVLCVNCFNPKDHIGHHVYTTICTEFN |
| | | NGECDCGDKTAWNHTLFCKAEEG |
| PS1379 | 722 | MHSKFSHAGRICGAKFKVGEPIYRCRECSFDVTCVLCVNCFNPKDHLGHHVYTTICTEFN |
| | | NGECDCGDKTAWNHTLFCKAEEG |
| PS1380 | 723 | MHSKFSHAGRICGAKFKVGEPIYRCRECSFDVTCVLCVNCFNPKDHIGHHVYTTICTEFN |
| | | NGECDCGDKTAWNHTLFCKAEEG |
| PS1381 | 724 | MHSKFSHAGRICGAKFKVGEPIYRCRECSFDVTCVLCVNCFNPKDHIGHHVYTTICTEFN |
| | | NGECDCGDKTAWNHTLFCKAEEG |
| PS1382 | 725 | MHSKFSHAGRICGAKFKVGEPIYRCRECSFDRTCVLCVNCFNPKDHIGHHVYTTICTEFN |
| | | NGECDCGDKTAWNHTLFCKAEEG |
| PS1383 | 726 | MHSKFSHAGRICGAKFKVGEPIYRCRECSFDRTCVLCVNCFNPKDHLGHHVYTTICTQKN |
| | | NGECDCGDKTAWNHTLFCKAEEG |
| PS1384 | 727 | MHSKFSHAGRICGAKFKVGEPIYRCRECSFDHTCVLCVNCFNPKDHLGHHVYTTICTEFN |
| | | NGECDCGDKTAWNHTLFCKAEEG |
| PS1385 | 728 | MHSKFSHAGRICGAKFKVGEPIYRCRECSFDHTCVLCVNCFNPKDHIGHHVYTTICTEFN |
| | | NGECDCGDKTAWNHTLFCKAEEG |
| PS1386 | 729 | MHSKFSHAGRICGAKFKVGEPIYRCRECSFDQTCVLCVNCFNPKDHLGHHVYTTICTEKN |
| | | NGECDCGDKTAWNHTLFCKAEEG |
| PS1387 | 730 | MHSKFSHAGRICGAKFKVGEPIYRCRLCSFDVTCVLCVNCFNPKDHLGHHVYTTIRTEFN |
| | | NGECDCGDKTAWNHTLFCKAEEG |
| PS1388 | 731 | MHSKFSHAGRICGAKFKVGEPIYRCRLCSFDVTCVLCVNCFNPKDHLGHHVYTTIRTEKN |
| | | NGECDCGDKTAWNHTLFCKAEEG |
| PS1389 | 732 | MHSKFSHAGRICGAKFKVGEPIYRCRLCSFDQTCVLCVNCFNPKDHLGHHVYTTIRTSFN |
| | | NGECDCGDKTAWNHTLFCKAEEG |
| PS1390 | 733 | MHSKFSHAGRICGAKFKVGEPIYRCKLCSFDVTCVLCVNCFNPKDHLGHHVYTTIRTSKN |
| | | NGECDCGDKTAWNHTLFCKAEEG |
| PS1391 | 734 | MHSKFSHAGRICGAKFKVGEPIYRCKECSFDVTCVLCVNCFNPKDHLGHHVYTTIRTEFN |
| | | NGECDCGDKTAWNHTLFCKAEEG |
| PS1392 | 735 | MHSKFSHAGRICGAKFKVGEPIYRCKECSFDDTCVLCVNCFNPKDHLGHHVYTTIRTEKN |
| | | NGECDCGDKTAWNHTLFCKAEEG |
| PS1393 | 736 | MHSKFSHAGRICGAKFKVGEPIYRCKLCSFDDTCVLCVNCFNPKDHLGHHVYTTIRTEFN |
| | | NGECDCGDKTAWNHTLFCKAEEG |
| PS1394 | 737 | MHSKFSHAGRICGAKFKVGEPIYRCRECSFDQTCVLCVNCFNPKDHLGHHVYTTICTEFN |
| | | NGECDCGDKTAWNHTLFCKAEEG |
| PS1395 | 738 | MHSKFSHAGRICGAKFKVGEPIYRCKECSFDVTCVLCVNCFNPKDHLGHHVYTTIRTEKN |
| | | NGECDCGDKTAWNHTLFCKAEEG |
| PS1396 | 739 | MHSKFSHAGRICGAKFKVGEPIYRCRLCSFDDTCVLCVNCFNPKDHLGHHVYTTIRTEFN |
| | | NGECDCGDKTAWNHTLFCKAEEG |
| PS1397 | 740 | MHSKFSHAGRICGAKFKVGEPIYRCKLCSFDVTCVLCVNCFNPKDHLGHHVYTTIRTEFN |
| | | NGECDCGDKTAWNHTLFCKAEEG |
| PS1398 | 741 | MHSKFSHAGRICGAKFKVGEPIYRCRECSFDHTCVLCVNCFNPKDHLGHHVYTTIRTDKN |
| | | NGECDCGDKTAWNHTLFCKAEEG |
| PS1399 | 742 | MGLENSLETLRFSISNLSMQTHAARMRTFMYWPSSVPVQPEQLASAGFYYLGRNDDVCCF |
| | | CCDGALRCWQSGDDPWVEHALWFPRCEFLIRMKGQEFVDEIQGRYPHLLEQLLSG |
| PS1400 | 743 | MGLENSLETLRFSISNLSMQTHAARMRTFMYWPSSVPVQPEQLASAGFYYVGRNDIVRCF |
| | | CCDGALWCWESGDDPWVEHAKWFPRCEFLIRMKGQEFVDEIQGRYPHLLEQLLSG |
| PS1401 | 744 | MGLENSLETLRFSISNLSMQTHAARMRTFMYWPSSVPVQPEQLASAGFYYLGRNDEVRCF |
| | | CCDGGLHCWQSGDDPWVEHALWFPRCEFLIRMKGQEFVDEIQGRYPHLLEQLLSG |
| PS1402 | 745 | MGLENSLETLRFSISNLSMQTHAARMRTFMYWPSSVPVQPEQLASAGFYYSGRNDEVRCF |
| | | CCDGVLHCWESGDDPWVEHAKHFPRCEFLIRMKGQEFVDEIQGRYPHLLEQLLSG |
| PS1403 | 746 | MGLENSLETLRFSISNLSMQTHAARMRTFMYWPSSVPVQPEQLASAGFYYSGRNDLVACF |
| | | CCDGGLTCWESGDDPWVEHAKHFPRCEFLIRMKGQEFVDEIQGRYPHLLEQLLSG |
| PS1404 | 747 | MGLENSLETLRFSISNLSMQTHAARMRTFMYWPSSVPVQPEQLASAGFYYLGRNDIVRCF |
| | | CCDGVLGCWESGDDPWVEHARHFPRCEFLIRMKGQEFVDEIQGRYPHLLEQLLSG |
| PS1405 | 748 | MGLENSLETLRFSISNLSMQTHAARMRTFMYWPSSVPVQPEQLASAGFYYLGRNDEVRCF |
| | | CCDGGLHCWQSGDDPWVEHARWFPRCEFLIRMKGQEFVDEIQGRYPHLLEQLLSG |
| PS1406 | 749 | MGLENSLETLRFSISNLSMQTHAARMRTFMYWPSSVPVQPEQLASAGFYYVGRNDIVRCF |
| | | CCDGALHCWKSGDDPWVEHARHFPRCEFLIRMKGQEFVDEIQGRYPHLLEQLLSG |
| PS1407 | 750 | MGLENSLETLRFSISNLSMQTHAARMRTKMYWESSVPVQWEQLASYGFQFVGRNDDVKCQ |
| | | CCDGGLRCWESGDDVAVEHSKRFIRCEFLIRMKGQEFVDEIQGRYPHLLEQLLSG |
| PS1408 | 751 | MGLENSLETLRFSISNLSMQTHAARMRTFMYWPSSVPVQPEQLASAGFYYLGRNDLVKCF |
| | | CCDGVLHCWQSGDDPWVEHARHFPRCEFLIRMKGQEFVDEIQGRYPHLLEQLLSG |
| PS1409 | 752 | MGLENSLETLRFSISNLSMQTHAARMRTFMYWPSSVPVQPEQLASAGFYYLGRNDIVKCF |
| | | CCDGVLHCWQSGDDPWVEHAKHFPRCEFLIRMKGQEFVDEIQGRYPHLLEQLLSG |
| PS1410 | 753 | MGLENSLETLRFSISNLSMQTHAARMRTFMYWPSSVPVQPEQLASAGFYYLGRNDEVRCF |
| | | CCDGVLHCWESGDDPWVEHARWFPRCEFLIRMNGQEFVDEIQGRYPHLLEQLLSG |
| PS1411 | 754 | MGLENSLETLRFSISNLSMQTHAARMRTFMYWPSSVPVQPEQLASAGFYYSGRNDIVRCF |
| | | CCDGDLHCWESGDDPWVEHAKWFPRCEFLIRMKGQEFVDEIQGRYPHLLEQLLSG |
| PS1412 | 755 | MGLENSLETLRFSISNLSMQTHAARMRTFMYWPSSVPVQPEQLASAGFYYAGRNDEVKCF |
| | | CCDGGLHCWESGDDPWVEHARHFPRCEFLIRMNGQEFVDEIQGRYPHLLEQLLSG |
| PS1413 | 756 | MGLENSLETLRFSISNLSMQTHAARMRTFMYWPSSVPVQPEQLASAGFYYLGRNDIVKCF |
| | | CCDGVLHCWQSGDDPWVEHARHFPRCEFLIRMKGQEFVDEIQGRYPHLLEQLLSG |
| PS1414 | 757 | MGLENSLETLRFSISNLSMQTHAARMRTFMYWPSSVPVQPEQLASAGFYYLGRNDLVKCF |
| | | CCDGVLHCWQSGDDPWVEHAKHFPRCEFLIRMKGQEFVDEIQGRYPHLLEQLLSG |
| PS1415 | 758 | MGLENSLETLRFSISNLSMQTHAARMRTFMYWPSSVPVQPEQLASAGFYYLGRNDLVKCF |
| | | CCDGVLHCWQSGDDPWVEHAKWFPRCEFLIRMKGQEFVDEIQGRYPHLLEQLLSG |
| PS1416 | 759 | MGLENSLETLRFSISNLSMQTHAARMRTFMYWPSSVPVQPEQLASAGFYYLGRNDLVKCF |
| | | CCDGVLHCWESGDDPWVEHARHFPRCEFLIRMKGQEFVDEIQGRYPHLLEQLLSG |
| PS1417 | 760 | MGLENSLETLRFSISNLSMQTHAARMRTFMYWPSSVPVQPEQLASAGFYYLGRNDIVKCF |
| | | CCDGVLHCWQSGDDPWVEHAKWFPRCEFLIRMKGQEFVDEIQGRYPHLLEQLLSG |
| PS1418 | 761 | MGLENSLETLRFSISNLSMQTHAARMRTFMYWPSSVPVQPEQLASAGFYYLGRNDLVKCF |
| | | CCDGALHCWQSGDDPWVEHARHFPRCEFLIRMKGQEFVDEIQGRYPHLLEQLLSG |
| PS1419 | 762 | MGLENSLETLRFSISNLSMQTHAARMRTFMYWPSSVPVQPEQLASAGFYYLGRNDLVKCF |
| | | CCDGVLHCWQSGDDPWVEHARHFPRCEFLIRMKGQEFVDEVQGRYPHLLEQLLSG |
| PS1420 | 763 | MGLENSLETLRFSISNLSMQTHAARMRTFMYWPSSVPVQPEQLASAGFYYLGRNDLVKCF |
| | | CCDGVLHCWQSGDDPWVEHARHFPRCEFLIRMKGQEFVDEIQGRYPHLLEQLPSG |
| PS1421 | 764 | MGLENSLETLRFSISNLSMQTHAARMRTFMYWPSSVPVQPEQLASAGFYYLGRNDLVKCF |
| | | CCDGVLRCWESGDDPWVEHAKHFPRCEFLIRMKGQEFVDEIQGRYPHLLEQLLSG |
| PS1422 | 765 | MGLENSLETLRFSISNLSMQTHAARMRTFMYWPSSVPVQPEQLASAGFYYLGRNDLVKCF |
| | | CCDGVLHCWQSGDDPWVEHATWFPRCEFLIRMKGQEFVDEIQGRYPHLLEQLLSG |
| PS1423 | 766 | MGLENSLETLRFSISNLSMQTHAARMRTFMYWPSSVPVQPEQLASAGFYYGGRNDLVKCF |
| | | CCDGVLHCWQSGDDPWVEHARHFPRCEFLIRMKGQEFVDEIQGRYPHLLEQLLSG |
| PS1424 | 767 | MGLENSLETLRFSISNLSMQTHAARMRTFMYWPSSVPVQPEQLASAGFYYLGRNDLVKCF |
| | | CCDGVLHCWQGGDDPWVEHARHFPRCEFLIRMKGQEFVDEIQGRYPHLLEQLLSG |
| PS1425 | 768 | MPTAASATESAIEDTPAPARPEVDGRTKPKRQPRYHVVLWDDDDHTYAYVVVMLVSLFGH |
| | | PPSRGYRMAKEMDVQGRVIVLTTTRAHAELKRDQIHAFGRDRLLARSKGSMKASIEAEE |
| PS1426 | 769 | MPTAASATESAIEDTPAPARPEVDGRTKPKRQPRYHVVLWDDDDHTYAYVVVMLRSLFGH |
| | | PPSRGYRMAKEMDVQGRVIVLTTTRAHAELKRDQIHAFGRDRLLARSKGSMKASIEAEE |
| PS1427 | 770 | MPTAASATESAIEDTPAPARPEVDGRTKPKRQPRYHVVLWDDDDHTYQYVVTMLRSLFGH |
| | | PPSRGYRMAKEMDTQGRVIVLTTTREHAELKRDQIHAFGRDRLLARSKGSMKASIEAEE |
| PS1428 | 771 | MPTAASATESAIEDTPAPARPEVDGRTKPKRQPRYHVVLWDDDDHTYQYVVVMLRSLFGH |
| | | PPGRGYRMAKEMDTQGRVIVLTTTREHAELKRDQIHAFGRDRLLARSKGSMKASIEAEE |
| PS1429 | 772 | MPTAASATESAIEDTPAPARPEVDGRTKPKRQPRYHVVLWDDDDHTYQYVVVMLRSLFGH |
| | | PPSRGYRIAKEMDTQGRVIVLTTTREHAELKRDQIHAFGRDRLLARSKGSMKASIEAEE |
| PS1430 | 773 | MPTAASATESAIEDTPAPARPEVDGRTKPKRQPRYHVVLWDDDDHTYQYVVVMLRSLFGH |
| | | PPSRGYRMAKEVDTQGRVIVLTTTREHAELKRDQIHAFGRDRLLARSKGSMKASIEAEE |
| PS1431 | 774 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAEFKRDQIHAFGRDRLLARSKGSMKASIEAEE |
| PS1432 | 775 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQINAFGRDRLLARSKGSMKASIEAEE |
| PS1433 | 776 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAFGRDSLLARSKGSMKASIEAEE |
| PS1434 | 777 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAFGRDGLLARSKGSMKASIEAEE |
| PS1435 | 778 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAFGRDRLLARSKGSMTASIEAEE |
| PS1436 | 779 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAWGRDSLLARSKGSMKASIEAEE |
| PS1437 | 780 | MPTAASATESAIEDTPAPARSEVDGYTVPKRQPRYHVVLWDDDDHTYQYVVGMLRSVFGH |
| | | PPSRGYRMAKEMDTQGRVIVLTTTREHAELKRDQIHAFGRDRLLARSKGSMKASIEAEE |
| PS1438 | 781 | MPTAASATESAIEDTPAPVRPEVDGYTVPKRQPRYHVVLWDDDDHTYQYVVTMLRSLFGH |
| | | PPGRGYRMAKEMDTQGRVIVLTTTREHAELKRDQIHAFGRDRLLARSKGSMKASIEAEE |
| PS1439 | 782 | MPTAASATESAMEDTPAPARPEVDGRTKPKRQPRYHVVLWNDDDHTYQYVVVMLQSLFGH |
| | | PPKRGYRMAKEMDTQGRVIVLTTTREHAELKRDQIHAFGRDRLLARCKGSMKASIEAEE |
| PS1440 | 783 | MPTAASATESAIEDTPAPARPEVDGRTKPKRQPRYHVVLWDDDDHTYQYVVVMLQSLFGH |
| | | PPNRGYRMAKEMDTQGRVIVLTTTREHAELKRDQIHAFGCDRLLARCKGSMKASIEAEE |
| PS1441 | 784 | MPTAASATESAIEDPPAPARPEVDGRTKPKRQPRYHVVMWEDDDHTYQYVVVMLRSLFGH |
| | | PPNRGYRMAKEMDTQGRVIVLTTTREHAELKRDQIHAFGCDRLLARSKGSMKASIEAEE |
| PS1442 | 785 | MPTAASATESAIEDTPAPARPEVDGRTKPKRQPRYHVVLWDDDDHTYQYVVVMLRSLFGH |
| | | PPSRGYRMAKELDTQGRVIVLTTTREHAELKRDQIRAYGRDGLLARSKGSMKASIEAEE |
| PS1443 | 786 | MPTAASATESAIEDTPAPARSEVDGRTEPKRQPRYHVVLWDDDDHTYQYVVAMLRSLFGH |
| | | PPSRGYRMAKEMDTQGRVIVLTTTREHAELKRDQIHAFGRDSLLARSKGSMKASIEAEE |
| PS1444 | 787 | MPTAASATESAIEDTPAPARPEVDGRTKPKRQPRYHVVLWDDDDHTYQYVVVMLRSLFGH |
| | | SASRGYRMAKEMDTQGRVIVLTTTREHAELKRDQIHAFGRDRLLARSKGSMKASIEAEE |
| PS1445 | 788 | MPTAASATESAIEDTPAPARSEVDGYTVPKRQPRYHVVLWDDDDHTYQYVVVMLRSLFGH |
| | | PPSRGYRMAKEMDTQGRVIVLTTTREHAELKRDQIHAFGRDRLLARSKGSMKASIEAEE |
| PS1446 | 789 | MPTAASATESAIEDTPAPARPEVDGRTKPKRQPRYHVVLWDDDDHTYQYVVHMLRSIFGH |
| | | PPSRGYRMAKEMDTQGRVIVLTTTREYAELKRDQIHAFGRDRLLARSKGSMKASIEAEE |
| PS1447 | 790 | MPTAASATESAIEDTPAPARPEVDGRTKPKRQPRYHVVLWDDDDHTYQYVVVMLRSLFGH |
| | | PPERGYRMAKEMDTQGRVIVLTTTREHAELKRDQIHAFGRDRLLARSKGSMKASIEAEE |
| PS1448 | 791 | MPTAASATESAIEDTPAPARPEVDGRTKPKRQPRYHVVLWDDDDHTYQYVVVMLRSLFGH |
| | | PPSRGYRMAKEMDTQGRVIVLTTTREHAELKRDQIHAFGYDRLLARSKGSMKASIEAEE |
| PS1457 | 792 | MNGLSAQHERIAPARHECVYTSCYSEENIWKLCEHIKTSKRCPLGDVYAVFISNERKMVPIWK |
| | | LKSGRGEEPVIWDYNVILLHDCHKEQTFIYDLDTTLPFPCPFDTYVKEAFKSDNYINPAFWRK |
| | | LRVVPADVFLQNFASDRSHNKDASGGWRMPPPPYPCIETAESRMNLDDFISMNPSVGWGHVYT |
| | | LEEFVQHFGKT |
| PS1458 | 793 | MNGLSAQHERIAPARHECVYTSCYSEENVWKLCEHIKTSKRCPLGDVYAVFISNERKMVPIWK |
| | | KKSGRGEELLIWDYNVIILHDCHKEQTFIYDLDTTLPFPCPFDTYVKEAFKSDNYINPAFWRK |
| | | LRVVPADVFLQNFASDRSHQKDASGGWRMPPPPYPCIETAESRMNLDDFISMNPSVGWGHVYT |
| | | LEEFVQHFGKT |
| PS1459 | 794 | MNGLSAQHERIAPARHECVYTSCYAEENVWKVCEHIKTSKRCPLGDVYAVFISNERKMVPIWK |
| | | RKSGRGEEFIIWDYQVIFLHDCHKEQTFIYDLDTTLPFPCPFDTYVKEAFKSDNYINPAFWRK |
| | | LRVVPADVFLQNFASDRSHNKDASGGWRMPPPPYPCIETAESRMNLDDFISMNPSVGWGHVYT |
| | | LEEFVQHFGKT |
| PS1460 | 795 | MNGLSAQHERIAPARHECVYTSCYAEENAWKFCEHIKTSKRCPLGDVYAVFISNERKMVPIWK |
| | | KKSGRGEEPVIWDYQVIVLHDCHKEQTFIYDLDTTLPFPYPFDTYVKEAFKSDNYINPAFWRK |
| | | LRVVPADVFLQNFASDRSHNKDASGGWRMPPPPYPCIETAESRMNLDDFISMNPSVGWGHVYT |
| | | LEEFVQHFGKT |
| PS1461 | 796 | MNGLSAQHERIAPARHECVYTSCYAEENAWKMCEHIKTSKKCPLGDVYAVFISNERKMVPIWK |
| | | KKSGQGEEFLIWDYNVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1462 | 797 | MNGLSAQHERIAPARHECVYTSCYAEENLFKICEHIKTSKRCPLGDVYAVFISNERKMVPIWK |
| | | PKSGRGEEVIIWDYNVIVLHDCHKEQTFIYDLDTTLPFPCPFDTYVKEAFKSDNYINPAFWRK |
| | | LRVVPADVFLQNFASDRSHNKDASGGWRMPPPPYPCIETAESRMNLDDFISMNPSVGWGHVYT |
| | | LEEFVQHFGKT |
| PS1463 | 798 | MNGLSAQHERIAPARHECVYTSCYAEENLFKICEHIKTSKRCPLGDVYAVFISNERKMVPIWK |
| | | PKSGRGEEVIIWDYNVIVLHDCHKEQTFIYDLDTTLPFPCPFDTYVKEAFKSDNYINHAFWRK |
| | | LRVVPADVFLQNFASDRSHNKDASGGWRMPPPPYPCIETAESRMNLDDFISMNPSVGWGHVYT |
| | | LEEFVQHFGKT |
| PS1464 | 799 | MNGLSAQHERIAPARHECVYTSCYSEENVWKLCEHIKTSKRCPLGDVYAVFISNERKMVPIWK |
| | | LKSGQGEEPVIWDYQVILLHDCHKEQTFIYDLDTTLPFPCPFDTYVKEAFKSDNYINHAFWRK |
| | | LRVVPADVFLQNFASDRSHNKDASGGWRMPPPPYPCIETAESRMNLDDFISMNPSVGWGHVYT |
| | | LEEFVQHFGKT |
| PS1465 | 800 | MNGLSAQHERIAPARHECVYTSCYSEENVWKLCEHIKTSKRCPLGDVYAVFISNERKMVPIWK |
| | | LKSGQGEEPVIWDYQVILLHDCHKEQTFIYDLDTTLPFPCPFDTYVKEAFKSDNYINHAFWRK |
| | | LRVVPADVFLQNFASDRSHQKDASGGWRMPPPPYPCIETAESRMNLDDFISMNPSVGWGHVYT |
| | | LEEFVQHFGKT |
| PS1466 | 801 | MNGLSAQHERIAPARHECVYTSCYAEENAWKFCEHIKTSKRCPLGDVYAVFISNERKMVPIWK |
| | | KKSGRGEEPVIWDYQVIVLHDCHKEQTFIYDLDTTLPFPYPFDTYVKEAFKSDNYINPAFWRK |
| | | LRVVPADVFLQNFASDRSHQKDASGGWRMPPPPYPCIETAESRMNLDDFISMNPSVGWGHVYT |
| | | LEEFVQHFGKT |
| PS1467 | 802 | MNGLSAQHERIAPARHECVYTSCYSEENASKLCEHIKTSKRCPLGDVYAVFISNERKMVPIWK |
| | | QKSGRGEEPVIWDYHVIILHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1468 | 803 | MNGLSAQHERIAPARHECVYTSCYAEENLFKICEHIKTSKRCPLGDVYAVFISNERKMVPIWK |
| | | PKSGRGEEVIIWDYNVIVLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1469 | 804 | MNGLSAQHERIAPARHECVYTSCYSEENVWKACEHIKTSKRCPLGDVYAVFISNERKMVPIWK |
| | | KKSGRGEEPVIWDYHVIVLHDCHKEQTFIYDLDTTLPFPCPFDTYVKEAFKSDNYINPAFWRK |
| | | LRVVPADVFLQNFASDRSHNKDASGGWRMPPPPYPCIETAESRMNLDDFISMNPSVGWGHVYT |
| | | LEEFVQHFGKT |
| PS1470 | 805 | MNGLSAQHERIAPARHECVYTSCYAEENAWKFCEHIKTSKRCPLGDVYAVFISNERKMVPIWK |
| | | PKSGRGEEVIIWDYNVIVLHDCHKEQTFIYDLDTTLPFPCPFDTYVKEAFKSDNYINPAFWRK |
| | | LRVVPADVFLQNFASDRSHQKDASGGWRMPPPPYPCIETAESRMNLDDFISMNPSVGWGHVYT |
| | | LEEFVQHFGKT |
| PS1471 | 806 | MNGLSAQHERIAPARHECVYTSCYAEENLFKICEHIKTSKRCPLGDVYAVFISNERKMVPIWK |
| | | LKSGQGEEPVIWDYQVILLHDCHKEQTFIYDLDTTLPFPCPFDTYVKEAFKSDNYINHAFWRK |
| | | LRVVPADVFLQNFASDRSHNKDASGGWRMPPPPYPCIETAESRMNLDDFISMNPSVGWGHVYT |
| | | LEEFVQHFGKT |
| PS1472 | 807 | MNGLSAQHERIAPARHECVYTSCYSEENVWKLCEHIKTSKRCPLGDVYAVFISNERKMVPIWK |
| | | LKSGQGEEPVIWDYQVILLHDCHKEQPFIYDLDTTLPFPCPFDTYVKEAFKSDNYINHAFWRK |
| | | LRVVPADVFLQNFASDRSHNKDASGGWRMPPPPYPCIETAESRMNLDDFISMNPSVGWGHVYT |
| | | LEEFVQHFGKT |
| PS1473 | 808 | MNGLSAQHERIAPARHECVYTSCYAEENLWKACEHIKTSKRCPLGDVYAVFISNERKMVPIWK |
| | | KKSGRGEEFIIWDYNVIVLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1474 | 809 | MNGLSAQHERIAPARHECVYTSCYAEENLFKICEHIKTSKRCPLGDVYAVFISNERKMVPIWK |
| | | PKSGRGEEVIIWDYNVIVLHDCHKEQTFNYDLDTTLPFPCPFDTYVKEAFKSDNYINPAFWRK |
| | | LRVVPADVFLQNFASDRSHQKDASGGWRMPPPPYPCIETAESRMNLDDFISMNPSVGWGHVYT |
| | | LEEFVQHFGKT |
| PS1475 | 810 | MNGLSAQHERIAPARHECVYTSCYAEENAWKFCEHIKTSKRCPLGDVYAVFISNERKMVPIWK |
| | | KKSGRGEEPVIWDYQVIVLHDCHKEQPFIYDLDTTLPFPYPFDTYVKEAFKSDNYINPAFWRK |
| | | LRVVPADVFLQNFASDRSHNKDASGGWRMPPPPYPCIETAESRMNLDDFISMNPSVGWGHVYT |
| | | LEEFVQHFGKT |
| PS1476 | 811 | MNGLSAQHERIAPARHECVYTSCYAEENLFKICEHIKTSKRCPLGDVYAVFISNERKMVPIWK |
| | | PKSGRGEEVIIWDYNVIVLHDCHKEQPFIYDLDTTLPFPCPFDTYVKEAFKSDNYINPAFWRK |
| | | LRVVPADVFLQNFASDRSHQKDASGGWRMPPPPYPCIETAESRMNLDDFISMNPSVGWGHVYT |
| | | LEEFVQHFGKT |
| PS1477 | 812 | MNGLSAQHERIAPARHECVYTSCYAEENAWKFCEHIKTSKRCPLGDVYAVFISNERKMVPIWK |
| | | LKSGQGEEPVIWDYQVILLHDCHKEQTFIYDLDTTLPFPCPFDTYVKEAFKSDNYINHAFWRK |
| | | LRVVPADVFLQNFASDRSHNKDASGGWRMPPPPYPCIETAESRMNLDDFISMNPSVGWGHVYT |
| | | LEEFVQHFGKT |
| PS1478 | 813 | MNGLSAQHERIAPARHECVYTSCYAEENAWKFCEHIKTSKRCPLGDVYAVFISNERKMVPIWK |
| | | RKSGRGEEPVIWDYQVIVLHDCHKEQTFIYDLDTTLPFPCPFDTYVKEAFKSDNYINPAFWRK |
| | | LRVVPADVFLQNFASDRSHQKDASGGWRMPPPPYPCIETAESRMNLDDFISMNPSVGWGHVYT |
| | | LEEFVQHFGKT |
| PS1480 | 814 | MNGLSAQHERIAPARHECVYTSCYAEENLFKICEHIKTSKRCPLGDVYAVFISNERKMVPIWK |
| | | PKSGRGEEVIIWDYNVIVLHDCHKEQTFIYDLDTTLPFPCPFDTYVKEAFKSDNYINPAFWRK |
| | | LRVVPADVFLQNFASDRSHQKDASGGWRMPPPPYPCIETAESRMNLDDFISMNPSVGWGHVYT |
| | | LEEFVQHFGKT |
| PS1481 | 815 | MNGLSAQHERIAPARHECVYTSCYSEENVWKLCEHIKTSKRCPLGDVYAVFISNERKMVPIWK |
| | | RKSGRGEEPVIWDYQVILLHDCHKEQTFIYDLDTTLPFPCPFDTYVKEAFKSDNYINPAFWRK |
| | | LRVVPADVFLQNFASDRSHNKDASGGWRMPPPPYPCIETAESRMNLDDFISMNPSVGWGHVYT |
| | | LEEFVQHFGKT |
| PS1482 | 816 | MNGLSAQHERIAPARHECVYTSCYSEENVWKLCEHIKTSKRCPLGDVYAVFISNERKMVPIWK |
| | | KKSGRGEELLIWDYNVIILHDCHKEQTFIYDLDTTLPFPCPFDTYVKEAFKSDNYINPAFWRK |
| | | LRVVPADVFLQNFASDRSHNKDASGGWRMPPPPYPCIETAESRMNLDDFISMNPSVGWGHVYT |
| | | LEEFVQHFGKT |
| PS1483 | 817 | MNGLSAQHERIAPARHECVYTSCYAEENVFKACEHIKTSKSCPLGDVYAVFISNERKMVPIWK |
| | | QKSGRSEEFIIWDYQVIILHDCHKEQTFIYDLDTTLPFPCPFDTYVKEAFKSDNYINPAFWRK |
| | | LRVVPADVFLQNFASDRSHQKDASGGWRMPPPPYPCIETAESRMNLDDFISMNPSVGWGHVYT |
| | | LEEFVQHFGKT |
| PS1484 | 818 | MNGLSAQHERIAPARHECVYTSCYAEENLWKLCEHIKTSKRCPLGDVYAVFISNERKMVPIWK |
| | | PKSGRGEELWIWDYQVIILHDCHKEQTFIYDLDTTLPFPCPFDTYVKEAFKSDNYINPAFWRK |
| | | LRVVPADVFLQNFASDRSHNKDASGGWRMPPPPYPCIETAESRMNLDDFISMNPSVGWGHVYT |
| | | LEEFVQHFGKT |
| PS1485 | 819 | MNGLSAQHERIAPARHECVYTSCYSEENVWKLCEHIKTSKRCPLGDVYAVFISNERKMVPIWK |
| | | PKSGRGEEVIIWDYNVIVLHDCHKEQTFIYDLDTTLPFPCPFDTYVKEAFKSDNYINPAFWRK |
| | | LRVVPADVFLQNFASDRSHQKDASGGWRMPPPPYPCIETAESRMNLDDFISMNPSVGWGHVYT |
| | | LEEFVQHFGKT |
| PS1486 | 820 | MNGLSAQHERIAPARHECVYTSCYSEENVWKLCEHIKTSKRCPLGDVYAVFISNERKMVPIWK |
| | | LKSGQGEEPVIWDYQVILLHDCHKEQTFIYDLDTTLPFPCPFDTYVKEAFKSDNYINPAFWRK |
| | | LRVVPADVFLQNFASDRSHNKDASGGWRMPPPPYPCIETAESRMNLDDFISMNPSVGWGHVYT |
| | | LEEFVQHFGKT |
| PS1487 | 821 | MNGLSAQHERIAPARHECVYTSCYSEENVWKLCEHIKTSKRCPLGDVYAVFISNERKMVPIWK |
| | | LKSGQGEEPVIWDYQVILLHDCHKEQTFIYDLDTTLPFPCPFDTYVKEAFKSDNYINPAFWRK |
| | | LRVVPADVFLQNFASDRSHQKDASGGWRMPPPPYPCIETAESRMNLDDFISMNPSVGWGHVYT |
| | | LEEFVQHFGKT |
| PS1488 | 822 | MNGLSAQHERIAPARHECVYTSCYSKENLWKLCEHIKTSKRCPLGDVYAVFISNERKMVPIWK |
| | | KKSGRGEELWIWDYNVIVLHDCHKEQTFIYDLDTTLPFPCPFDTYVKEAFKSDNYINPAFWRK |
| | | LRVVPADVFLQNFASDRSHTKDASGGWRMPPPPYPCIETAESRMNLDDFISMNPSVGWGHVYT |
| | | LEEFVQHFGKT |
| PS1489 | 823 | MNGLSAQHERIAPARHECVYTSCYSEENAWKMCEHIKTSKRCPLGDVYAVFISNERKMVPIWK |
| | | QKSGRGEEPVIWDYNVIFLHDCHKEQTFIYDLDTALPFPCPFDTYVKEAFKSDNYINPAFGRK |
| | | LRVVPADVFLQNFASDCSHQKDASGGWRMPPPPYPCIETAESRMNLDDFISMNPSVGWGHVYT |
| | | LEEFVQHFGKT |
| PS1490 | 824 | MNGLSAQHERIAPARHECVYTSCYAEENAWKFCEHIKTSKRCPLGDVYAVFISNERKMVPIWK |
| | | RKSGRGEEPVIWDYQVIVLHDCHKEQTFIYDLDTTLPFPCPFDTYVKEAFKSDNCINPAFWRK |
| | | LRVVPADVFLQNFASDRSHQKDASGGWRMPPPPYPCIETAESRMNLDDFISMNPSVGWGHVYT |
| | | LEEFVQHFGKT |
| PS1491 | 825 | MNGLSAQHERIAPARHECVYTSCYSEENVWKLCEHIKTSKRCPLGDVYAVFISNERKMVPIWK |
| | | KKSGRGEEFLIWDYNVIVLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1492 | 826 | MNGLSAQHERIAPARHECVYTSCYSEENLFKMCEHIKTSKRCPLGDVYAVFISNERKMVPIWK |
| | | RKSGRGEEPLIWDYHVIFLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1493 | 827 | MNGLSAQHERIAPARHECVYTSCYSEENAWKMCEHIKTSKRCPLGDVYAVFISNERKMVPIWK |
| | | QKSGRGEEIWIWDYHVIILHDCHKEQTFIYDLDTTLPFPCPFDTYVKEAFKSDNYINPAFWRK |
| | | LRVVPADVFLQNFASDRSHNKDASGGWRMPPPPYPCIETAESRMNLDDFISMNPSVGWGHVYT |
| | | LEEFVQHFGKT |
| PS1494 | 828 | MNGLSAQHERIAPARHECVYTSCYSEENVWKLCEHIKTSKRCPLGDVYAVFISNERKMVPIWK |
| | | QKSGRGEEFLIWDYNVIVLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1495 | 829 | MNGLSAQHERIAPARHECVYTSCYAEENAWKMCEHIKTSKRCPLGDVYAVFISNERKMVPIWK |
| | | RKSGRGEEPIIWDYHVIILHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1496 | 830 | MNGLSAQHERIAPARHECVYTSCYAEENIWKACVHIKTSKRCPLGDVYVVFISNERKMVPIWK |
| | | QKSGRGEEFLIWDYNVIILHDCHKEQTLIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1497 | 831 | MNGLSAQHERIAPARHECVYTSCYSEENAWKMCEHIKTSKRCPLGDVYAVFISNERKMVPIWK |
| | | QKSGRGEEPVIWDYHVIVLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1498 | 832 | MNGLSAQHERIAPARHECVYTSCYAEENIWKACVHIKTSKRCPLGDVYVVFISNERKMVPIWK |
| | | QKSGRGEEFLIWDYNVIILHDCHKEQTFIYDLDTTLPFPCPFDTCVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1499 | 833 | MNGLSAQHERIAPARHECVYTSCYAEENIWKACVHIKTSKRCPLGDVYVVFISNERKMVPIWK |
| | | QKSGRGEEFLIWDYNVIILHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1587 | 834 | MGLENSLETLRFSISNLSMQTHAARMRTFMYWPSSVPVQPEQLASAGFYYVGRNDDVKCFCCD |
| | | GGLRCWESGDDPWVEHAKWFPRCEFLIRMKGQEFVDEIQGRYPHLLEQLLSEAAAKEAAAKEA |
| | | AAKMGLENSLETLRFSISNLSMQTHAARMRTFMYWPSSVPVQPEQLASAGFYYVGRNDDVKCF |
| | | CCDGGLRCWESGDDPWVEHAKWFPRCEFLIRMKGQEFVDEIQGRYPHLLEQLLSG |
| PS1599 | 835 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKTEEEKRKREEEEMNGLSAQHERIAPARHECVYTSCYSEENVWKLCEHIKTSKR |
| | | CPLGDVYAVFISNERKMVPIWKQKSGRGEEPVIWDYQVILLHDCHKEQTFIYDLDTTLPFPCP |
| | | FDTYVKEAFKSDNYINPAFWRKLRVVPADVFLQNFASDRSHMKDASGGWRMPPPPYPCIETAE |
| | | SRMNLDDFISMNPSVGWGHVYTLEEFVQHFGKT |
| PS1633 | 836 | MNGLSAQHERIAPARHECVYTSCYSEENVWKLCEHIKTSKRCPLGDVYAVFISNERKMVPIWK |
| | | QKSGRGEEPVIWDYRVILLHDCHKEQTFIYDLDTTLPFPCPFDTYVKEAFKSDNYINPAFWRK |
| | | LRVVPADVFLQNFASDRSRLKDASGGWRMPPPPYPCIETAESRMNLDDFISMNPSVGWGHVYT |
| | | LEEFVQHFGKT |
| PS1634 | 837 | MNGLSAQHERIAPARHECVYTSCYSEENVWKLCEHIKTSKRCPLGDVYAVFISNERKMVPIWK |
| | | QKSGRGEEPVIKDYAVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1635 | 838 | MNGLSAQHERIAPARHECVYTSCYSEENVWKLCEHIKTSKRCPLGDVYAVFISNERKMVPIWK |
| | | QKSGRGEEPVIWFYSVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1636 | 839 | MNGLSAQHERIAPARHECVYTSCYSEENVWKLCEHIKTSKRCPLGDVYAVFISNERKMVPIWK |
| | | QKSGRGEEPVIIFYHVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1637 | 840 | MNGLSAQHERIAPARHECVYTSCYSEENVWKLCEHIKTSKRCPLGDVYAVFISNERKMVPIWK |
| | | QKSGRGEEPVKWDYHVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1638 | 841 | MNGLSAQHERIAPARHECVYTSCYSEENVWKLCEHIKTSKRNPLGDVYAVFISNERKMVPIWK |
| | | QKSGRGEEPVIWDYHVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1639 | 842 | MNGLSAQHERIAPARHECVYTSCYSEENVWKLCEHIKTSKRCPLGDVYAVFISNERKMVPIWK |
| | | QKSGRGEEPVIWDYHVCLLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1640 | 843 | MNGLSAQHERIAPARHECVYTSCYSEENVWKLCEHIKTSKRCPLGDVYAVFISNERKMVPIWK |
| | | QKSGRGEEPVIWDYHVILLHDCHKEQTFIYDNDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1641 | 844 | MNGLSAQHERIAPARHECVYTSCYSEENVWKLCEHIKTSKRCPLGDVYAVFISNERKMVPIWK |
| | | QKSGRGEEPVIWDYHVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEQFVQHFGKT |
| PS1642 | 845 | MNGLSAQHERIAPARHECVYTSCYSEENVWKLCEHIKTSKRCPLGDVYAVFISNERKMVPIWK |
| | | QKSGRGEEPVIWDYHVILLHDCHKEQTFIYDLDTTLPFPCPFDTYVKEAFKSDNYINPAFWRK |
| | | LRVVPADVFLQNFASDRSHMKDASGGWRMPPPPYPCIETGESRMNLDDFISMNPSVGWGHVYT |
| | | LEEFVQHFGKT |
| PS1643 | 846 | MNGLSAQHERIAPARHECVYTSCYSEENVWKLCEHIKTSKRCPLGDVYAVFISNERKMVPIWK |
| | | QKSGRGEEPVIWDYHVILLHDCHKEQTFIYDLDTTLPFPCPFDTYVKETFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1644 | 847 | MNGLSAQHERIAPARHECVYTSCYSEENVWKLCEHIKTSKRCPLGDVYAVFISNERKMVPIWK |
| | | QKSGRGEEPVIWDYHVILLHDCHKEQTFIYDLDTTLPFPCPFDTYVKETFKSDNYINPAFWRK |
| | | LRVVPADVFLQNFASDRSRLKDASGGWRMPPPPYPCIETAESRMNLDDFISMNPSVGWGHVYT |
| | | LEEFVQHFGKT |
| PS1645 | 848 | MNGLSAQHERIAPARHECVYTSCYSEENVWKLCEHIKTSKRNPLGDVYAVFISNERKMVPIWK |
| | | QKSGRGEEPVIWFYAVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1646 | 849 | MNGLSAQHERIAPARHECVYTSCYSEENVWKLCEHIKTSKRNPLGDVYAVFISNERKMVPIWK |
| | | QKSGRGEEPVIWDYHVILLHDCHKEQTFIYDLDTTLPFPCPFDTYVKEAFKSDNYINPAFWRK |
| | | LRVVPADVFLQNFASDRSRLKDASGGWRMPPPPYPCIETAESRMNLDDFISMNPSVGWGHVYT |
| | | LEEFVQHFGKT |
| PS1647 | 850 | MNGLSAQHERIAPARHECVYTSCYSEENVWKLCEHIKTSKRCPLGDVYAVFISNERKMVPIWK |
| | | QKSGRGEEPVIWFYHVILLHDCHKEQTFIYDLDTTLPFPCPFDTYVKETFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1648 | 851 | MNGLSAQHERIAPARHECVYTSCYSEENVWKLCEHIKTSKRCPLGDVYAVFISNERKMVPIWK |
| | | QKSGRGEEPVIWFYHVILLHDCHKEQTFIYDLDTTLPFPCPFDTYVKETFKSDNYINPAFWRK |
| | | LRVVPADVFLQNFASDRSHMKDASGGWRMPPPPYPCIETAESRMNLDDFISMNPSVGWGHVYT |
| | | LEQFVQHFGKT |
| PS1649 | 852 | MNGLSAQHERIAPARHECVYTSCYSEENVWKLCEHIKTSKRCPLGDVYAVFISNERKMVPIWK |
| | | QKSGRGEEPVIWFYHVILLHDCHKEQTFIYDLDTTLPFPCPFDTYVKEAFKSDNYINPAFWRK |
| | | LRVVPADVFLQNFASDRSHMKDASGGWRMPPPPYPCIETGESRMNLDDFISMNPSVGWGHVYT |
| | | LEEFVQHFGKT |
| PS1650 | 853 | MNGLSAQHERIAPARHECVYTSCYSEENVWKLCEHIKTSKRCPLGDVYAVFISNERKMVPIWK |
| | | QKSGRGEEPVIWFYAVILLHDCHKEQTFIYDLDTTLPFPCPFDTYVKETFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1651 | 854 | MNGLSAQHERIAPARHECVYTSCYSEENVWKLCEHIKTSKRCPLGDVYAVFISNERKMVPIWK |
| | | QKSGRGEEPVIWDYHVILLHDCHKEQTFIYDLDTTLPFPCPFDTYVKEAFKSDNYINPAFWRK |
| | | LRVVPADVFLQNFASDRSRMKDASGGWRMPPPPYPCIETGESRMNLDDFISMNPSVGWGHVYT |
| | | LEEFVQHFGKT |
| PS1652 | 855 | MNGLSAQHERIAPARHECVYTSCYSEENVWKLCEHIKTSKRCPLGDVYAVFISNERKMVPIWK |
| | | QKSGRGEEPVIWDYHVILLHDCHKEQTFIYDLDTTLPFPCPFDTYVKEAFKSDNYINPAFWRK |
| | | LRVVPADVFLQNFASDRSRLKDASGGWRMPPPPYPCIETAESRMNLDDFISMNPSVGWGHVYT |
| | | LEEFVQHFGKT |
| PS1653 | 856 | MNGLSAQHERIAPARHECVYTSCYSEENVWKLCEHIKTSKRCPLGDVYAVFISNERKMVPIWK |
| | | QKSGRGEEPVIWDYHVILLHDCHKEQTFIYDLDTTLPFPCPFDTYVKEAFKSDNYINPAFWRK |
| | | LRVVPADVFLQNFASDRSRLKDASGGWRMPPPPYPCIETGESRMNLDDFISMNPSVGWGHVYT |
| | | LEEFVQHFGKT |
| PS1654 | 857 | MNGLSAQHERIAPARHECVYTSCYSEENVWKLCEHIKTSKRCPLGDVYAVFISNERKMVPIWK |
| | | QKSGRGEEPVIWDYHVILLHDCHKEQTFIYDLDTTLPFPCPFDTYVKEAFKSDNYINPAFWRK |
| | | LRVVPADVFLQNFASDRSRLKDASGGWRMPPPPYPCIETAESRMNLDDFISMNPSVGWGHVYT |
| | | LEQFVQHFGKT |
| PS1655 | 858 | MNGLSAQHERIAPARHECVYTSCYSEENVWKLCEHIKTSKRCPLGDVYAVFISNERKMVPIWK |
| | | QKSGRGEEPVIWDYHVILLHDCHKEQTFIYDLDTTLPFPCPFDTYVKEAFKSDNYINPAFWRK |
| | | LRVVPADVFLQNFASDRSHLKDASGGWRMPPPPYPCIETGESRMNLDDFISMNPSVGWGHVYT |
| | | LEEFVQHFGKT |
| PS1656 | 859 | MNGLSAQHERIAPARHECVYTSCYSEENVWKLCEHIKTSKRCPLGDVYAVFISNERKMVPIWK |
| | | QKSGRGEEPVIWFYHVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1737 | 860 | MNGLSAQHERIAPARHECVYTSCYSEENVWKLCEHIKTSKRCPLGDVYAVFISNERKMVPIWK |
| | | QRSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1738 | 861 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPRFWRK |
| | | LRVVPADVFLQNFASDRSHMKDASGGWRMPPPPYPCIETAESRMNLDDFISMNPSVGWGHVYT |
| | | LEEFVQHFGKT |
| PS1739 | 862 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYILPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1740 | 863 | MNGLSAQHERIAPARHECVYTSYYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1741 | 864 | MNGLSAQHERIAPARHECVYTSCYSEENVWKLCEHIKTSKRCPLGDFYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1742 | 865 | MNGLSAQHERIAPARHECVYTFCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1743 | 866 | MNGLSAQHERIAPARHECVYTSCYSEENVWKLCEHIKTSKRCPLGDVYAVFISNERKMVPIWK |
| | | QRSGRGEEPVIWDYQVILLHDCHKEQTFIYDLDTTLPFPCPFDTYVKEAFKSDNYINPRFWRK |
| | | LRVVPADVFLQNFASDRSHMKDASGGWRMPPPPYPCIETAESRMNLDDFISMNPSVGWGHVYT |
| | | LEEFVQHFGKT |
| PS1744 | 867 | MNGLSAQHERITPARHECVYTSCYSEENVWKLCEHIKTSKRCPLGDVYAVFISNERKMVPIWK |
| | | QRSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1745 | 868 | MNGLSAQHERITPARHECVYTFCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1746 | 869 | MNGLSAQHERIAPARHECVYTPCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1747 | 870 | MNGLSAQHERVAPARHECVYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1748 | 871 | MNGLSAQHERILPARHECVYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1749 | 872 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVRADVFLQNFASDRSHMKDASGGWRMPPPPYPCIETAESRMNLDDFISMNPSVGWGHVYT |
| | | LEEFVQHFGKT |
| PS1750 | 873 | MNGLSAQHERIAPARHECVYTSCYSEENVWKLCEHIKTSKRCPLGDVYAVFISNERKMVPIWK |
| | | QKVGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1751 | 874 | MNGLSAQHERIAPARHECVYTSCYSEENVWKLCEHIKTSKRCPLGDVYAVFISNERKMVPIWK |
| | | QKSGRGERPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1752 | 875 | MNGLSAQHERIAPARHECVYTSCYSEENVWKLCEHIKTSKRCPLGDVYAVFISNERKMVPIWK |
| | | QKSGRGEEPVIWDYQVILLHDTHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1753 | 876 | MNGLSAQHERIAPARHECVYTSCYSEENVWKLCEHIKTSKRCPLGDVYAVFISNERKMVPIWK |
| | | QKSGRGEEPVIWDYQVILLHACHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1754 | 877 | MNGLSAQHERIAPARHECVYTSCYSEENVWKLCEHIKTSKRCPLGDVYAVFISNERKMVPIWK |
| | | QKSGRGGEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1755 | 878 | MNGLSAQHERIAPARHECVYTSCYSEENVWKLCEHIKTSKRCPLGDVYAVFISNERKMVPIWK |
| | | QKSGRGNEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1756 | 879 | MNGLSAQHERIAPARHECVYTSCYSEENVWKLCEHIKTQKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1757 | 880 | MNGLSAQHERIAPARHECVYTSCYSEENVWKLCEHIKTSKRCPLGDVYAVFISNERKMVPIWK |
| | | QKSGRGEEPVIWDYQVILLHDPHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1758 | 881 | MNGLSAQHERIAPARHECVYTSCYSEENVWKLCEHIKTSKRCPLGDVYAVFISNERKMVPIWK |
| | | QKSCRGEEPVIWDYQVILLHDCHKEQTFIYDLDTTLPFPCPFDTYVKEAFKSDNYINPRFWRK |
| | | LRVVPADVFLQNFASDRSHMKDASGGWRMPPPPYPCIETAESRMNLDDFISMNPSVGWGHVYT |
| | | LEEFVQHFGKT |
| PS1821 | 882 | MNGLSAQHERIAPARHECVYTSCYTEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1822 | 883 | MNGLSAQHERIAPARHECVYTSCYYEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1823 | 884 | MNGLSAQHERIAPARHECVYTSCYSEENVWKLCEHIKTSKRCPLGDVYAVFISNEQKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1824 | 885 | MNGLSAQHERIAPARHECVYTSCYSEENVWKLCEHIKTSKRCPLGDVYAVFISNELKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1825 | 886 | MNGLSAQHERIAPARHECVYTSCYSEENVWKLCEHIKTSKRCPLGDVYAVFISNEIKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1826 | 887 | MNGLSAQHERIAPARHECVYTSCYSEENVWKLCEHIKTSKRCPLGDVYAVFISNERNMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1827 | 888 | MNGLSAQHERIAPARHECVYTSCYSEENVWKLCEHIKTSKRCPLGDVYAVFISNERLMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1828 | 889 | MNGLSAQHERIAPARHECVYTSCYSEENVWKLCEHIKTSKRCPLGDVYAVFISNEREMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1829 | 890 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWQMPPPPYPCIETAESRMNLDDFISMNPSVGWGHVYT |
| | | LEEFVQHFGKT |
| PS1830 | 891 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWLMPPPPYPCIETAESRMNLDDFISMNPSVGWGHVYT |
| | | LEEFVQHFGKT |
| PS1831 | 892 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWCMPPPPYPCIETAESRMNLDDFISMNPSVGWGHVYT |
| | | LEEFVQHFGKT |
| PS1832 | 893 | MNGLSAQHERIAPARHECVYTSCYCEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1833 | 894 | MNGLSAQHERILPARHECVYTSCYSEENVWKLCEHIKTSKRCPLGDVYAVFISNERKMVPIWK |
| | | QRSGRGEEPVIWDYQVILLHDCHKEQTFIYDLDTTLPFPCPFDTYVKEAFKSDNYIRPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1834 | 895 | MNGLSAQHERITPARHECVYTSFYSEENVWKLCEHIKTSKRCPLGDVYAVFISNERKMVPIWK |
| | | QKVGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1835 | 896 | MNGLSAQHERIALARHECVYTSCYSEENVWKLCEHIKTSKLCPLGDVYAVFISNERKMVPIWK |
| | | QKSCRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1836 | 897 | MNGLSAQHERILPARHECVYTSCYSEENVWKLCEHIKTSKRCPLGDVYAVFISNERKMVPIWK |
| | | QRSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1837 | 898 | MNGLSAQHERIAPARHECVYTSYYSEENVWKLCEHIKTQKRCPLGDVYAVFISNERKMVPIWK |
| | | QKSCRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1838 | 899 | MNGLSAQHERIALARHECIYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYILPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1839 | 900 | MNGLSAQHERIAPARHECVYTSCYSEENVWKLCEHIKTQKRCPLGDVYAVFISNERKMVPIWK |
| | | QKSGRGEEPVIWDYQVILLHDTHKEQTFIYDLDTTLPFPCPFDTYVKEAFKSDNYIRPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1840 | 901 | MNGLSAQHERIAPARHECVYTSFYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1841 | 902 | MNGLSAQHERIAPARHECVYTSCYSEENVWKLCEHIKTSKLCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1842 | 903 | MNGLSAQHERIAPARHECVYTSCYSEENVWKLCEHIKTSKRCPLGDVYAVFISNERKMVPIWK |
| | | QKSGRGEEPVIWDYRVILLHDCHKEQTFIYDLNTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1843 | 904 | MNGLSAQHERIAPARHECVYTSCYSEENVWKLCEHIKTSKRCPLGDVYAVFISNERKMVPIWK |
| | | QKSGRGEEPVIWDYRVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1844 | 905 | MNGLSAQHERIAPARHECVYTPCYGEENIWHLCQHIKTSKRCPLGDVYAVFISNERKMVPIWK |
| | | QKSGRGEEPIIWDYHVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1845 | 906 | MNGLSAQHERIAPARHECVYTSCYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDSSGGWRMPPPPYPCIETAESRMNLDDFISMNPSVGWGHVYT |
| | | LEEFVQHFGKT |
| PS1846 | 907 | MNGLSAQHERIAPARHECVYTSCYSEENVWKLCEHIKTSKRCPLGDVYAVFISNERKMVPIWK |
| | | QKSGRGEEPVFWDYQVILLHDCHKEQTFIYDLNTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1847 | 908 | MNGLSAQHERIAPARHECVYTSQYSEENVWKLCEHIKTSKRCPLGDVYAVFISNERKMVPIWK |
| | | QKSGRGEEPVIWDYQVILLHDCHKEQTFIYDLNTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1848 | 909 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLNTTLPFPCPFDTYVKEAFKSDNYINPAFWRK |
| | | LRVVPADVFLQNFASDRSHMKDAWGGWRMPPPPYPCIETAESRMNLDDFISMNPSVGWGHVYT |
| | | LEEFVQHFGKT |
| PS1849 | 910 | MNGLSAQHERIAPARHECVYTSCYSEVNVWKLCEHIKTSKRCPLGDVYAVFISNERKMVPIWK |
| | | QKSGRGEEPVIWDYQVIALHDCHKEQTFIYDLNTTLPFPCPFDTYVKETFKSDNYINPAFWRK |
| | | LRVVPADVFLQNFASDRSHMKDDSGGWRMPPPPYPCIETAESRMNLDDFISMNPSVGWGHVYT |
| | | LEEFVQHFGKT |
| PS1850 | 911 | MNGLSAQHERIAPARHECVYTSCYSEGNVWKLCEHIKTSKRNPLGNVYAVFISNERKMVPIWK |
| | | QKSGRGEEPVIWDYWVILLHDCHKEQTFIYDLDTTLPFPCPFDTYVKETFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1851 | 912 | MNGLSAQHERIAPARHECVYTSCYSWENVWKLCEHIKTSKRNPLGDVYAVFISNERKMVPIWK |
| | | QKSGRGEEPVIWDYQVGLLHDCHKEQTFIYDLDTTLPFPCPFDTYVKETFKSDNYINPAFWRK |
| | | LRVVPADVFLQNFASDRSHMKLASGGWRMPPPPYPCIETAESRMNLDDFISMNPSVGWGHVYT |
| | | LEEFVQHFGKT |
| PS1852 | 913 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLNTTLPFPCPFDTYVKEAFKSDNYINPAFWRK |
| | | LRVVPADVFLQNFASDRSHMKDARGGWRMPPPPYPCIETAESRMNLDDFISMNPSVGWGHVYT |
| | | LEEFVQHFGKT |
| PS1853 | 914 | MNGLSAQHERIAPARHECVYTSCYSEENVWKLCEHIKTSKRCPLGDVYAVFISNERKMVPIWK |
| | | QKSGRGEEPVIWDYQAILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1854 | 915 | MNGLSAQHERIAPARHECVYTSCTSKENVWKLCEHIKTSKRCPLGDVYAVFISNERKMVPIWK |
| | | QKSGRGEEPVIWFYQVILLHDCHKEQTFIYDLNTTLPFPCPFDTYVKEAFKSDNYINPAFWRK |
| | | LRVVPADVFLQNFASDRSHMKDAAGGWRMPPPPYPCIETAESRMNLDDFISMNPSVGWGHVYT |
| | | LEEFVQHFGKT |
| PS1855 | 916 | MEGNGPAAVHYQPASPPRDACVYTACYGEENIWHLCQYIKNHDQYPLEECYAVFISNERKMIP |
| | | IWKQQARPGDGPIIWDYHVLLLHVSSGGQSFIYDLDTVLPFPCLFDTYVEDAIKSDDDIHPQF |
| | | RRKFRVICADSYLKNFASDRSHMKDSSGNWREPPPPYPCIETGDSKMNLNDFISMDPKVGWGA |
| | | VYTLSEFTHRFGSKN |
| PS1856 | 917 | MEGNGPAAVHYQPASPPRDACVYTACYAEENIWHLCQYIKNHDQYPLEECYAVFISNERKMIP |
| | | IWKQQARPGDGPIIWDYHVVLLHVSSGGQSFIYDLDTVLLFPCLFDTYVEDAIKSDDDIHPQF |
| | | RRKFRVICADSYLKNFASDRSHMKDSSGNWREPPPPYPCIETGDSKMNLNDFISMDPKVGWGA |
| | | VYTLSEFTHRFGSKN |
| PS1857 | 918 | MEGNGPAAVHYQPASPPRDACVYSSCYSEENVWKLCEYIKNHDQYPLEECYAVFISNERKMIP |
| | | IWKQQARPGDGPVIWDYRVVLLHVSSGGQSFIYDLDTVLPFPCLFDTYVEDAIKSDDDILPQF |
| | | RRKFRVICADSYLKNFASDRSHMKDSSGNWREPPPPYPCIETGDSKMNLNDFISMDPKVGWGA |
| | | VYTLSEFTHRFGSKN |
| PS1858 | 919 | MEGNGPAAVHYQPASPPRDAGVYSSCYSEENVWKLCEYIKNHDQYPLEKCYAVFISNERKMIP |
| | | IWKQQARPGDGPVIRDYQVVLLHVSSGGQSFIYDLDTVLPFPCLFDTYVEDAIKSDDDIHPQF |
| | | RRKFRVICADSYLKNFASDRSHMKDSSGNWREPPPPYPCIETGDSKMNLNDFISMDPKVGWGA |
| | | VYTLSEFTHRFGSKN |
| PS1859 | 920 | MEGNGPAAVHYQPASPPRDACVYSSCYSEENVWKLCEYIKNHDQYPLEECYAVFISSERKMIP |
| | | IWKQQARPGDGPVIWDYRVVLLHVSSGGQSLIYDLDTVLPFPCLFDTYVEDAIKSDDDIHPQF |
| | | RRKFRVICADSYLKNFASDRSHMKDSSGNWREPPPPYPCIETGDSKMNLNDFISMDPKVGWGA |
| | | VYTLSEFTHRFGSKN |
| PS1860 | 921 | MEGNGPAAVHYQPASPPRDACVYSSCYSEENVWKLCEYIKNHDQYPLEECYAVFISNERKMIP |
| | | IWKQQARPGDGPVIWDYRVVLLHVSSGGQSFIYDLDTVLPFPCLFDTYVEDTIESDDDIHPQF |
| | | RRKFRVICADSYLKNFASDRSHMKDSSGNWREPPPPYPCIETGDSKMNLNDFISMDPKVGWGA |
| | | VYTLSEFTHRFGSKN |
| PS1861 | 922 | MEGNGPAAVHYQPASPPRDACVYSSCYSEENVWKLCEYIKNHDQYPLEGCYAVFISNERKMIP |
| | | IWKQQARPGDGPVIWDYQVVLLHVSSGGQSFIYDLDTVLPFPCLFDTYVEDTIKSDDDIHSQF |
| | | RRKFRVICADSYLKNFASDRSHMKDSSGNWREPPPPYPCIETGDSKMNLNDFISMDPKVGWGA |
| | | VYTLSEFTHRFGSKN |
| PS1862 | 923 | MEGNGPAAVHYQPASPPRDACVYSPCYGEENVWKLCEYIKNHDQYPLEECYAVFISNERKMIP |
| | | IWKQQARPGDGPIIWDYHVVLLHVSSGGQSFIYDLDTVLPFPCLFDTYVEDAIKSDDDIHPQF |
| | | RRKFRVICADSYLKNFASDRSHMKDSSGNWREPPPPYPCIETGDSKMNLNDFISMDPKVGWGA |
| | | VYTLSEFTHRFGSKN |
| PS1863 | 924 | MEGNGPAAVHYQPASPPRDACVYSSCYREENVWKLCEYIKNHDQYPLEECYAVFISNERKIIP |
| | | IWKQQARPGDGPVIWDYQVVLLHVSSGGQSFIYDLDTVLPFPCLFDTYVEDAIMSDDDIHPQF |
| | | RRKFRVICADSYLKNFASDRSHMKDSSGNWREPPPPYPCIETGDSKMNLNDFISMDPKVGWGA |
| | | VYTLSEFTHRFGSKN |
| PS1864 | 925 | MEGNGPAAVHYQPASPPRDACVYSSCYSEENVWKLCEYIKNHDLYPLEECYAVFISNERKMIP |
| | | IWKQQARPGDGPVIWDYQVVLLHVSSGGQSFIYDLDTVLPFPCLFDTYVEDAIKSDDDIHPQF |
| | | RRKFRVICADSYLKNFASDRSHMKDSSGNWLEPPPPYPCIETGDSKMNLNDFISMDPKVGWGA |
| | | VYTLSEFTHRFGSKN |
| PS1865 | 926 | MEGNGPAAVHYQPASPPRDACVYSSCYSEENVWKLCEYIKNHDQYPLEECYAVFISNERKMIP |
| | | IWKQQARPGDGPVIWDYRVVLLHVSSGGQSFIYDLDTVLPFPCLFDTYVEDAIKSDDDIHPQF |
| | | RRKFRVICADSYLKNFASDRSHMKDSSGNWREPPPPYPCIETGDSKMNLNDFISMDPKVGWGA |
| | | VYTLSEFTHRFGSKN |
| PS1866 | 927 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLNTTLPFPCPFDTYVKEAFKSDNYINPAFWRK |
| | | LRVVPADVFLQNFASDRSHMKDGSGGWRMPPPPYPCIETAESRMNLDDFISMNPSVGWGHVYT |
| | | LEEFVQHFGKT |
| PS1867 | 928 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLNTTLPFPCPFDTYVKEAFKSDNYINPAFWRK |
| | | LRVVPADVFLQNFASDRSHMKDVSGGWRMPPPPYPCIETAESRMNLDDFISMNPSVGWGHVYT |
| | | LEEFVQHFGKT |
| PS1868 | 929 | MNGLSAQHERIAPARHECVYTSCYSEENVWKLCEHIKTSKRCPLGDVYAVFISNERKMVPIWK |
| | | QKSGRGEEPVFWDYQVILLHDCHKEQTFIYDLNTTLPFPCPFDTYVKEAFKSDNYINPAFWRK |
| | | LRVVPADVFLQNFASDRSHMRDASGGWRMPPPPYPCIETAESRMNLDDFISMNPSVGWGHVYT |
| | | LEEFVQHFGKT |
| PS1869 | 930 | MNGLSAQHERIAPARHECVYTSCYSEENVWKLCEHIKTSKRCPLGDVYAVFISNERKMVPIWK |
| | | QKSGRGEEAVIWDYQVILLHDCHKEQTFIYDLNTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1870 | 931 | MNGLSAQHERIAPARHECVYTSGYSEENVWKLCEHIKTSKRCPLGDVYAVFISNERKMVPIWK |
| | | QKSGRGEEPVFWDYQVILLHDCHKEQTFIYDLNTTLPFPCPFDTYVKEAFKSDNYINPAFWRK |
| | | LRVVPADVFLQNFASDRSHMRDASGGWRMPPPPYPCIETAESRMNLDDFISMNPSVGWGHVYT |
| | | LEEFVQHFGKT |
| PS1871 | 932 | MNGLSAQHERIAPARHECVYTSGYSEENVWKLCEHIKTSKRCPLGDVYAVFISNERKMVPIWK |
| | | QKSGRGEEPVWWDYQVILLHDCHKEQTFIYDLNTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1872 | 933 | MNGLSAQHERIAPARHECVYTSCYSEENVWKLCEHIKTSKRNPLGDVYAVFISNERKMVPIWK |
| | | QKSGRGEEPEIWDYQVILLHDCHKEQTFIYDLNTTLPFPCPFDTYVKETFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1873 | 934 | MNGLSAQHERIAPARHECVYTSCYSEENVWKLCEHIKTSKRCPLGDVYAVFISNERKMVPIWK |
| | | QKSGRGEEPVFWDYQVILLHDCHKEQTFIYDLNTTLPFPCPFDTYVKEAFKSDNYINPAFWRK |
| | | LRVVPADVFLQNFASDRSHMKDDSGGWRMPPPPYPCIETAESRMNLDDFISMNPSVGWGHVYT |
| | | LEEFVQHFGKT |
| PS1874 | 935 | MNGLSAQHERIAPARHECVYTSCYSEENVWKLCEHIKTSKRCPLGDVYAVFISNERKMVPIWK |
| | | QKSGRGEEPVWWDYQVILLHDCHKEQTFIYDLDTTLPFPCPFDTYVKEAFKSDNYINPAFWRK |
| | | LRVVPADVFLQNFASDRSHMKNASGGWRMPPPPYPCIETAESRMNLDDFISMNPSVGWGHVYT |
| | | LEEFVQHFGKT |
| PS1875 | 936 | MNGLSAQHERIAPARHECVYTSCYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1876 | 937 | MNGLSAQHERIAPARHECVYTSCYSEENVWKLCEHIKTSKRCPLGDVYAVFISNERKMVPIWK |
| | | QKSGRGEEPVWWDYQVILLHDCHKEQTFIYDLNTTLPFPCPFDTYVKEAFKSDNYINPAFWRK |
| | | LRVVPADVFLQNFASDRSHMKDESGGWRMPPPPYPCIETAESRMNLDDFISMNPSVGWGHVYT |
| | | LEEFVQHFGKT |
| PS1877 | 938 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFRSDNYINPAFWRK |
| | | LRVVPADVFLQNFASDRSHMKDASGGWCMPPPPYPCIETAESRMNLDDFISMNPSVGWGHVYT |
| | | LEEFVQHFGKT |
| PS1878 | 939 | MNGLSAQHERIAPARHECVYTFYYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1879 | 940 | MNGLSAQHERIAPARHECVYTFYYSEENVWKLCEHIKTSKRCPLGDVYAVFISNERKMVPIWK |
| | | QKSGRGERPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1880 | 941 | MNGLSAQHERIAPARHECVYTSYYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYIPPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1881 | 942 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYIPPRFWRK |
| | | LRVVPADVFLQNFASDRSHMKDPSGGWRMPPPPYPCIETAESRMNLDDFISMNPSVGWGHVYT |
| | | LEEFVQHFGKT |
| PS1882 | 943 | MNGLSAQHERIAPARHECVYTSCYSEENVWKLCEHIKTSKRCPLGDVYAVFISNERKMVPIWK |
| | | QKSCRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1883 | 944 | MNGLSAQHERIAPARHECVYTTCYSEENVYHLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1884 | 945 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDVSGGWRMPPPPYPCIETAESRMNLDDFISMNPSVGWGHVYT |
| | | LEEFVQHFGKT |
| PS1885 | 946 | MNGLSAQHERIAPARHECVYTSCYSEENVWKLCEHIKTSKRCPLGDVYAVFISNERKMVPIWK |
| | | QKSGRGEEPVFWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1886 | 947 | MNGLSAQHERIAPARHECVYTSCYSEENVWKLCEHIKTSKRCPLGDVYAVFISNERKMVPIWK |
| | | QKSGRGEEPVIWDYQVVLLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1887 | 948 | MNGLSAQHERIAPARHECVYTSCYSEENVWKLCEHIKTSKRCPLGDVYAVFISNERKMVPIWK |
| | | QKSGRGEECVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1888 | 949 | MNGLSAQHERIAPARHECVYTSCYGEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1889 | 950 | MNGLSAQHERIAPARHECVYTSCYSEENVWKLCEHIKTSKRCPLGDVYAVFISNERKMVPIWK |
| | | QKSGRGEEPCIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1890 | 951 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDCSGGWRMPPPPYPCIETAESRMNLDDFISMNPSVGWGHVYT |
| | | LEEFVQHFGKT |
| PS1891 | 952 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDRSGGWRMPPPPYPCIETAESRMNLDDFISMNPSVGWGHVYT |
| | | LEEFVQHFGKT |
| PS1892 | 953 | MNGLSAQHERIAPARHECVYTECYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1893 | 954 | MNGLSAQHERIAPARHECVYTSCYSEENVWKLCEHIKTSKRCPLGDVYAVFISNERKMVPIWK |
| | | QKSGRGEEPEIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1894 | 955 | MNGLSAQHERIAPARHECVYTSCYSEENVWKLCEHIKTSKRCPLGDVYAVFISNERKMVPIWK |
| | | QKSGRGEEPVIYDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS1895 | 956 | MNGLSAQHERIAPARHECVYTSCYSEENVWKLCEHIKTSKRCPLGDVYAVFISNERKMVPIWK |
| | | QKSGRGEEPVLWDYQVILLHDCHKEQTFIYDLDTTLPFPCPFDTYVKEAFKSDNYINPAFWRK |
| | | LRVVPADVFLQNFASDRSHMKDGSGGWRMPPPPYPCIETAESRMNLDDFISMNPSVGWGHVYT |
| | | LEEFVQHFGKT |
| PS1896 | 957 | MNGLSAQHERIAPARHECVYTSCYSEENVWKLCEHIKTSKRCPLGDVYAVFISNERKMVPIWK |
| | | QKSGRGEEPVVWDYQVILLHDCHKEQTFIYDLDTTLPFPCPFDTYVKEAFKSDNYINPAFWRK |
| | | LRVVPADVFLQNFASDRSHMKDESGGWRMPPPPYPCIETAESRMNLDDFISMNPSVGWGHVYT |
| | | LEEFVQHFGKT |
| PS1897 | 958 | MNGLSAQHERIAPARHECVYTSCYSEENVWKLCEHIKTSKRCPLGDVYAVFISNERKMVPIWK |
| | | QKSGRGEERVIWDYQVILLHDCHKEQTFIYDLDTTLPFPCPFDTYVKEAFKSDNYINPAFWRK |
| | | LRVVPADVFLQNFASDRSHMKDQSGGWRMPPPPYPCIETAESRMNLDDFISMNPSVGWGHVYT |
| | | LEEFVQHFGKT |
| PS1898 | 959 | MNGLSAQHERIAPARHECVYTSCYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRVHMKDASGGWRMPPPPYPCIETAESRMNLDDFISMNPSVGWGHVYT |
| | | LEEFVQHFGKT |
| PS2014 | 960 | MNGLSAQHERIAPARHECVYTSCYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDARGGWRMPPPPYPCIETAESRMNLDDFISMNPSVGWGHVYT |
| | | LEEFVQHFGKT |
| PS2015 | 961 | MNGLSAQHERIAPARHECVYTSCYGEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDARGGWRMPPPPYPCIETAESRMNLDDFISMNPSVGWGHVYT |
| | | LEEFVQHFGKT |
| PS2016 | 962 | MNGLSAQHERIAPARHECVYTSQYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2017 | 963 | MNGLSAQHERIAPARHECVYTSQYGEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2018 | 964 | MNGLSAQHERIAPARHECVYTSCYGEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2019 | 965 | MNGLSAQHERIAPARHECVYTPCYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2020 | 966 | MNGLSAQHERIAPARHECVYTSCYSEENVWKLCEHIKTSKRCPLGDVYAVFISNERKMVPIWK |
| | | QKSGRGEEPIIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2021 | 967 | MNGLSAQHERIAPARHECVYTSCYGEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDSSGGWRMPPPPYPCIETAESRMNLDDFISMNPSVGWGHVYT |
| | | LEEFVQHFGKT |
| PS2022 | 968 | MNGLSAQHERIAPARHECVYTSCYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDGSGGWRMPPPPYPCIETAESRMNLDDFISMNPSVGWGHVYT |
| | | LEEFVQHFGKT |
| PS2023 | 969 | MNGLSAQHERIAPARHECVYTSCYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDVSGGWRMPPPPYPCIETAESRMNLDDFISMNPSVGWGHVYT |
| | | LEEFVQHFGKT |
| PS2024 | 970 | MNGLSAQHERIAPARHECVYTSCYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDDSGGWRMPPPPYPCIETAESRMNLDDFISMNPSVGWGHVYT |
| | | LEEFVQHFGKT |
| PS2025 | 971 | MNGLSAQHERIAPARHECVYTSCYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDESGGWRMPPPPYPCIETAESRMNLDDFISMNPSVGWGHVYT |
| | | LEEFVQHFGKT |
| PS2026 | 972 | MNGLSAQHERIAPARHECVYTPCYGEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2027 | 973 | MNGLSAQHERIAPARHECVYTSCYGEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDSSGGWRMPPPPYPCIETAESRMNLDDFISMNPSVGWGHVYT |
| | | LEEFVQHFGKT |
| PS2028 | 974 | MNGLSAQHERIAPARHECVYTSCYGEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDARGGWRMPPPPYPCIETAESRMNLDDFISMNPSVGWGHVYT |
| | | LEEFVQHFGKT |
| PS2029 | 975 | MNGLSAQHERIAPARHECVYTSCYSEENVWHLCQHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2030 | 976 | MNGLSAQHERIAPARHECVYTPCYGEENVWKLCEHIKTSKRCPLGDVYAVFISNERKMVPIWK |
| | | QKSGRGEEPIIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2031 | 977 | MNGLSAQHERIAPARHECVYTSCYGEENVWHLCQHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2032 | 978 | MNGLSAQHERIAPARHECVYTPCYGEENIWKLCEHIKTSKRCPLGDVYAVFISNERKMVPIWK |
| | | QKSGRGEEPIIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2033 | 979 | MNGLSAQHERIAPARHECVYTPCYGEENVWHLCQHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2034 | 980 | MNGLSAQHERIAPARHECVYTSCYGEENIWHLCQHIKTSKRCPLGDVYAVFISNERKMVPIWK |
| | | QKSGRGEEPIIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2035 | 981 | MNGLSAQHERIAPARHECVYTPCYGEENIWHLCQHIKTSKRCPLGDVYAVFISNERKMVPIWK |
| | | QKSGRGEEPIIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2036 | 982 | MNGLSAQHERVAPARHECVYTPCYGEENVWKLAQHIKTSKRCPLGDVYAVFISNERKMVPIWK |
| | | QKSGRGEEPLVWDYHVLLLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2037 | 983 | MNGLSAQHERIAPARHECVYTACYGEENVWKLAQHIKTSKRCPLGDVYAVFISNERKMVPIWK |
| | | QKSGRGEEPIVWDYHVLLLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2038 | 984 | MNGLSAQHERIAPARHECVYTACYGEENVWHLCGHIKTSKRCPLGDVYAVFISNERKMVPIWK |
| | | QKSGRGEEPIIWDYHVLLLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2039 | 985 | MNGLSAQHERIAPARHECVYTACYGEENIWHLAQHIKTSKRCPLGDVYAVFISNERKMVPIWK |
| | | QKSGRGEEPVIWDYHVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2040 | 986 | MNGLSAQHERIAPARHECVYTPCYAEENVYKLAEHIKTSKRCPLGDVYAVFISNERKMVPIWK |
| | | QKSGRGEEPIIWDYQVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2041 | 987 | MNGLSAQHERIAPARHECVYTPCYGEENIWHLAQHIKTSKRCPLGDVYAVFISNERKMVPIWK |
| | | QKSGRGEEPVIWDYHVLLLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2042 | 988 | MNGLSAQHERIAPARHECVYTPCYGEENIWHLAQHIKTSKRCPLGDVYAVFISNERKMVPIWK |
| | | QKSGRGEEPILWDYHVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2043 | 989 | MNGLSAQHERIAPARHECVYTPCYGEENIWHLCEHIKTSKRCPLGDVYAVFISNERKMVPIWK |
| | | QKSGRGEEPILWDYHVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2044 | 990 | MNGLSAQHERIAPARHECVYTPCYGEENIWHLCQHIKTSKRCPLGDVYAVFISNERKMVPIWK |
| | | QKSGRGEEPILWDYHVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2045 | 991 | MNGLSAQHERIAPARHECVYTPCYGEENVYHLCEHIKTSKRCPLGDVYAVFISNERKMVPIWK |
| | | QKSGRGEEPIVWDYHVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2046 | 992 | MNGLSAQHERIAPARHECVYTACYGEENVWKLCQHIKTSKRCPLGDVYAVFISNERKMVPIWK |
| | | QKSGRGEEPILWDYHVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2047 | 993 | MNGLSAQHERIAPARHECVYTSCYSEENVWKLCEHIKTSKRCPLGDVYAVFISSERKMVPIWK |
| | | QKSGRGEEPVIWDYRVILLHDCHKEQTLIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2048 | 994 | MNGLSAQHERIAPARHECVYTSCYSEENVWKLCEHIKTSKRCPLGDVYAVFISNERKMVPIWK |
| | | QKSGRGEEPVIWDYRVILLHDCHKEQTFIYDLDTTLPFPCPFDTYVKETFESDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2049 | 995 | MNGLSAQHERIAPARHECVYTSCYSEENVWKLCEHIKTSKRCPLGGVYAVFISNERKMVPIWK |
| | | QKSGRGEEPVIWDYQVILLHDCHKEQTFIYDLDTTLPFPCPFDTYVKETFKSDNYINSAFWRK |
| | | LRVVPADVFLQNFASDRSHMKDASGGWRMPPPPYPCIETAESRMNLDDFISMNPSVGWGHVYT |
| | | LEEFVQHFGKT |
| PS2050 | 996 | MNGLSAQHERIAPARHECVYTPCYGEENVWKLCEHIKTSKRCPLGDVYAVFISNERKMVPIWK |
| | | QKSGRGEEPIIWDYHVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2051 | 997 | MNGLSAQHERIAPARHECVYTSCYREENVWKLCEHIKTSKRCPLGDVYAVFISNERKIVPIWK |
| | | QKSGRGEEPVIWDYQVILLHDCHKEQTFIYDLDTTLPFPCPFDTYVKEAFMSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2052 | 998 | MNGLSAQHERIAPARHECVYTSCYSEENVWKLCEHIKTSKLCPLGDVYAVFISNERKMVPIWK |
| | | QKSGRGEEPVIWDYQVILLHDCHKEQTFIYDLDTTLPFPCPFDTYVKEAFKSDNYINPAFWRK |
| | | LRVVPADVFLQNFASDRSHMKDASGGWLMPPPPYPCIETAESRMNLDDFISMNPSVGWGHVYT |
| | | LEEFVQHFGKT |
| PS2053 | 999 | MNGLSAQHERILPARHECVYTECYSEENVWKLCEYIKTSKRCPLGDVYAVFISNERKMVPIWK |
| | | QRSGRGERVVIWDYQVIMLHDCHKEQTFIYDLDTTLPFPCPFDTYVKEAFKSDNYINPRFWRK |
| | | LRVVRADVFLQNFASDRSHMKDASGGWRMPPPPYPCIETAESRMNLDDFISMNPSVGWGHVYT |
| | | LEEFVQHFGKT |
| PS2054 | 1000 | MNGLSAQHERILPARHECVYTPCYSEENVWKLCEHIKTQKRCPLGDVYAVFISNERKMVPIWK |
| | | QKVGRGEEPVWWDYQVILLHDCHKEQTFIYDLDTTLPFPCPFDTYVKEAFKSDNYIRPRFWRK |
| | | LRVVPADVFLQNFASDRSHMKDASGGWRMPPPPYPCIETAESRMNLDDFISMNPSVGWGHVYT |
| | | LEEFVQHFGKT |
| PS2055 | 1001 | MNGLSAQHERILPARHECVYTECYSEENVWKLCEHIKTSKRCPLGDVYAVFISNERKMVPIWK |
| | | QRSGRGERVVIWDYQVILLHDCHKEQTFIYDLDTTLPFPCPFDTYVKEAFKSDNYIRPRFWRK |
| | | LRVVPADVFLQNFASDRSHMKDASGGWRMPPPPYPCIETAESRMNLDDFISMNPSVGWGHVYT |
| | | LEEFVQHFGKT |
| PS2056 | 1002 | MNGLSAQHERILPARHECVYTPCYSEENVWKLCEHIKTQKRCPLGDVYAVFISNERKMVPIWK |
| | | QRSGRGERPVIWDYQVILLHDCHKEQTFIYDLDTTLPFPCPFDTYVKEAFKSDNYINPRFWRK |
| | | LRVVPADVFLQNFASDRSHMKDASGGWRMPPPPYPCIETAESRMNLDDFISMNPSVGWGHVYT |
| | | LEEFVQHFGKT |
| PS2057 | 1003 | MNGLSAQHERILPARHECVYTECYSEENVWKLCEHIKTSKRCPLGDVYAVFISNERKMVPIWK |
| | | QKSGRGEEPVIWDYQVILLHDCHKEQTFIYDLDTTLPFPCPFDTYVKEAFKSDNYIRPRFWRK |
| | | LRVVPADVFLQNFASDRSHMKDASGGWRMPPPPYPCIETAESRMNLDDFISMNPSVGWGHVYT |
| | | LEEFVQHFGKT |
| PS2116 | 1004 | MNGLSAQHERIAPARHECVYTECYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2117 | 1005 | MNGLSAQHERIAPARHECVYTSCYSEENVWKLCEHIKTSKRCPLGDVYAVFISNERKMVPIWK |
| | | QKSGRGEERVIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2118 | 1006 | MNGLSAQHERIAPARHECVYTSCYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDQSGGWRMPPPPYPCIETAESRMNLDDFISMNPSVGWGHVYT |
| | | LEEFVQHFGKT |
| PS2119 | 1007 | MNGLSAQHERIAPARHECVYTECYSEENVWKLCEHIKTSKRCPLGDVYAVFISNERKMVPIWK |
| | | QKSGRGEERVIWDYKVILLHDTHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2120 | 1008 | MNGLSAQHERIAPARHECVYTSCYSEENVWKLCEHIKTQKRCPLGDVYAVFISNERKMVPIWK |
| | | QKSGRGEEPVIWDYKVILLHDTHKEQTFIYDLDTTLPFPCPFDTYVKEAFKSDNYIRPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2121 | 1009 | MNGLSAQHERIAPARHECVYTECYSEENVWKLCEHIKTQKRCPLGDVYAVFISNERKMVPIWK |
| | | QKSGRGEEPVIWDYKVILLHDTHKEQTFIYDLDTTLPFPCPFDTYVKEAFKSDNYIRPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2122 | 1010 | MNGLSAQHERIAPARHECVYTECYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDQSGGWRMPPPPYPCIETAESRMNLDDFISMNPSVGWGHVYT |
| | | LEEFVQHFGKT |
| PS2123 | 1011 | MNGLSAQHERIAPARHECVYTECYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYIRPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2124 | 1012 | MNGLSAQHERIAPARHECVYTECYSEENVWKLCEHIKTSKRCPLGDVYAVFISNERKMVPIWK |
| | | QKSGRGEEPVIWDYKVILLHDTHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2125 | 1013 | MNGLSAQHERIAPARHECVYTECYSEENVWKLCEHIKTQKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2126 | 1014 | MNGLSAQHERIAPARHECVYTSCYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYIRPAFWRK |
| | | LRVVPADVFLQNFASDRSHMKDQSGGWRMPPPPYPCIETAESRMNLDDFISMNPSVGWGHVYT |
| | | LEEFVQHFGKT |
| PS2127 | 1015 | MNGLSAQHERIAPARHECVYTSCYSEENVWKLCEHIKTSKRCPLGDVYAVFISNERKMVPIWK |
| | | QKSGRGEEPVIWDYKVILLHDTHKEQTFIYDLDTTLPFPCPFDTYVKEAFKSDNYINPAFWRK |
| | | LRVVPADVFLQNFASDRSHMKDQSGGWRMPPPPYPCIETAESRMNLDDFISMNPSVGWGHVYT |
| | | LEEFVQHFGKT |
| PS2128 | 1016 | MNGLSAQHERIAPARHECVYTSCYSEENVWKLCEHIKTQKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDQSGGWRMPPPPYPCIETAESRMNLDDFISMNPSVGWGHVYT |
| | | LEEFVQHFGKT |
| PS2129 | 1017 | MNGLSAQHERIAPARHECVYTECYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYIRPAFWRK |
| | | LRVVPADVFLQNFASDRSHMKDQSGGWRMPPPPYPCIETAESRMNLDDFISMNPSVGWGHVYT |
| | | LEEFVQHFGKT |
| PS2130 | 1018 | MNGLSAQHERIAPARHECVYTECYSEENVWKLCEHIKTSKRCPLGDVYAVFISNERKMVPIWK |
| | | QKSGRGEEPVIWDYKVILLHDTHKEQTFIYDLDTTLPFPCPFDTYVKEAFKSDNYINPAFWRK |
| | | LRVVPADVFLQNFASDRSHMKDQSGGWRMPPPPYPCIETAESRMNLDDFISMNPSVGWGHVYT |
| | | LEEFVQHFGKT |
| PS2131 | 1019 | MNGLSAQHERIAPARHECVYTECYSEENVWKLCEHIKTQKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDQSGGWRMPPPPYPCIETAESRMNLDDFISMNPSVGWGHVYT |
| | | LEEFVQHFGKT |
| PS2132 | 1020 | MNGLSAQHERIAPARHECVYTECYSEENVWKLCEHIKTSKRCPLGDVYAVFISNERKMVPIWK |
| | | QKSGRGEEPVIWDYKVILLHDTHKEQTFIYDLDTTLPFPCPFDTYVKEAFKSDNYIRPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2133 | 1021 | MNGLSAQHERIAPARHECVYTECYSEENVWKLCEHIKTQKRCPLGDVYAVFISNERKMVPIWK |
| | | QKSGRGEEPVIWDYKVILLHDCHKEQTFIYDLDTTLPFPCPFDTYVKEAFKSDNYIRPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2134 | 1022 | MNGLSAQHERIAPARHECVYTECYSEENVWKLCEHIKTSKRCPLGDVYAVFISNERKMVPIWK |
| | | QKSGRGEEPVIWDYKVILLHDCHKEQTFIYDLDTTLPFPCPFDTYVKEAFKSDNYIRPAFWRK |
| | | LRVVPADVFLQNFASDRSHMKDVSGGWRMPPPPYPCIETAESRMNLDDFISMNPSVGWGHVYT |
| | | LEEFVQHFGKT |
| PS2135 | 1023 | MNGLSAQHERIAPARHECVYTECYSEENVWKLCEHIKTQKRCPLGDVYAVFISNERKMVPIWK |
| | | QKSGRGEEPVIWDYKVILLHDTHKEQTFIYDLDTTLPFPCPFDTYVKEAFKSDNYINPAFWRK |
| | | LRVVPADVFLQNFASDRSHMKDASGGWRMPPPPYPCIETAESRMNLDDFISMNPSVGWGHVYT |
| | | LEEFVQHFGKT |
| PS2136 | 1024 | MNGLSAQHERIAPARHECVYTECYSEENVWKLCEHIKTSKRCPLGDVYAVFISNERKMVPIWK |
| | | QKSGRGEEPVIWDYKVILLHDTHKEQTFIYDLDTTLPFPCPFDTYVKEAFKSDNYINPAFWRK |
| | | LRVVPADVFLQNFASDRSHMKDVSGGWRMPPPPYPCIETAESRMNLDDFISMNPSVGWGHVYT |
| | | LEEFVQHFGKT |
| PS2137 | 1025 | MNGLSAQHERIAPARHECVYTECYSEENVWKLCEHIKTQKRCPLGDVYAVFISNERKMVPIWK |
| | | QKSGRGEEPVIWDYKVILLHDCHKEQTFIYDLDTTLPFPCPFDTYVKEAFKSDNYINPAFWRK |
| | | LRVVPADVFLQNFASDRSHMKDVSGGWRMPPPPYPCIETAESRMNLDDFISMNPSVGWGHVYT |
| | | LEEFVQHFGKT |

TABLE 2
Non-limiting examples of tag sequences.

| | SEQ | |
|---|---|---|
| Name | ID NO: | Sequence |
| Biotinylation tag | 1026 | GGGSGGGSGGGSGLN |
| | | DFFEAQKIEWHE |
| Bis-biotinylation | 1027 | GGGSGGGSGGGSGLN |
| tag | | DFFEAQKIEWHEGGG |
| | | SGGGSGGGSGLNDFF |
| | | EAQKIEW |
| | | HE |
| Bis-biotinylation | 1028 | GSGGGSGGGSGGGSG |
| tag | | LNDFFEAQKIEWHEG |
| | | GGSGGGSGGGSGLND |
| | | FFEAQKI |
| | | EWHE |
| His/biotinylation | 1029 | GHHHHHHHHHHGGGS |
| tag | | GGGSGGGSGLNDFFE |
| | | AQKIEWHE |
| His/bis- | 1030 | GHHHHHHHHHHGGGS |
| biotinylation | | GGGSGGGSGLNDFFE |
| tag | | AQKIEWHEGGGSGGG |
| | | SGGGSGL |
| | | NDFFEAQKIEWHE |
| His/bis- | 1031 | GGSHHHHHHHHHHGG |
| biotinylation | | GSGGGSGGGSGLNDF |
| tag | | FEAQKIEWHEGGGSG |
| | | GGSGGGS |
| | | GLNDFFEAQKIEWHE |
| His/bis- | 1032 | GSHHHHHHHHHHGGG |
| biotinylation | | SGGGSGGGSGLNDFF |
| tag | | EAQKIEWHEGGGSGG |
| | | GSGGGSG |
| | | LNDFFEAQKIEWHE |
| Bis-biotinylation/ | 1033 | GGGSGGGSGGGSGLN |
| His tag | | DFFEAQKIEWHEGGG |
| | | SGGGSGGGSGLNDFF |
| | | EAQKIEW |
| | | HEGHHHHHH |

### EXAMPLES

### Example 1. Real-Time Dynamic Single-Molecule Protein Sequencing on an Integrated Semiconductor Device

In this example, a novel method that consists of a dynamic sequencing-by-degradation approach in which single surface-immobilized peptide molecules are probed in real time by a mixture of dye-labeled N-terminal amino acid recognizers was demonstrated. By measuring fluorescence intensity, lifetime, and intermolecular kinetics of recognizers on a novel semiconductor chip, the ability to annotate amino acids and collectively identify the peptide sequence was shown. Leveraging the kinetics of binding allows each recognizer to uniquely identify multiple amino acids. Also described here are the principles and processes to expand the number of recognizable amino acids. Furthermore, it was shown that this method is compatible with both synthetic peptides and natural peptides isolated from recombinant human proteins, and capable of detecting single amino acid changes and post-translational modifications. The results demonstrated a robust core technology that can serve as an accurate, sensitive, and scalable next-generation sequencing platform for proteins.

Measurements of the proteome provide deep and valuable insight into biological processes. However, methods with higher sensitivity are needed to fully understand the complex and dynamic states of the proteome in cells and changes to the proteome that occur in disease states, and to make this information more accessible. The complex nature of the proteome and the chemical properties of proteins present several fundamental challenges to achieving comprehensive sensitivity, throughput, and adoption on par with DNA sequencing technologies. These challenges include the large number of different proteins per cell (>10,000) and yet larger number of proteoforms; the very wide dynamic range of protein abundance in cells and biological fluids and lack of correlation with transcript levels; the costs and high detection limits of current mass spectrometry methods²; and the inability to copy or amplify proteins. Methods to directly sequence single protein molecules offer the maximum possible detection sensitivity, with the potential to enable single-cell inputs, digital quantification based on read counts, detection of post-translational modifications (PTMs) and low-abundance or aberrant proteoforms, and cost and throughput levels that favor broad adoption.

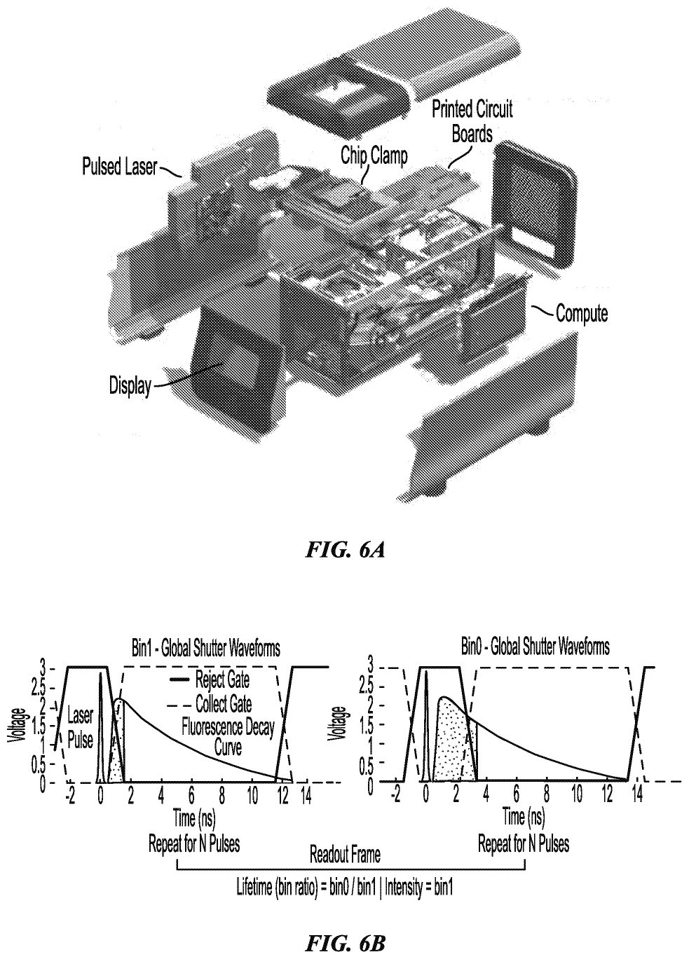

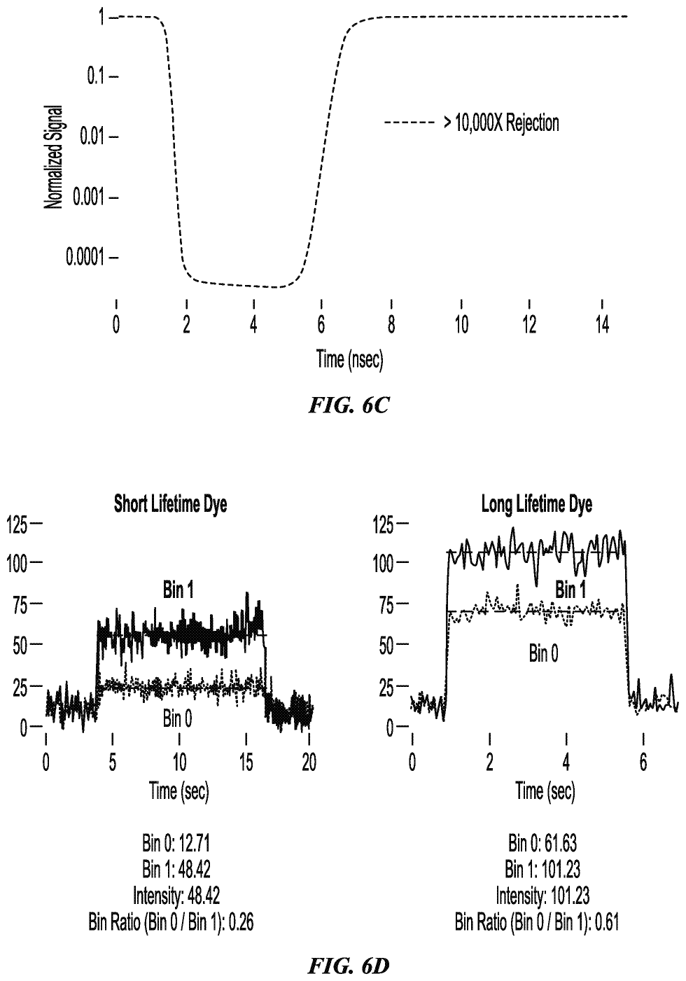

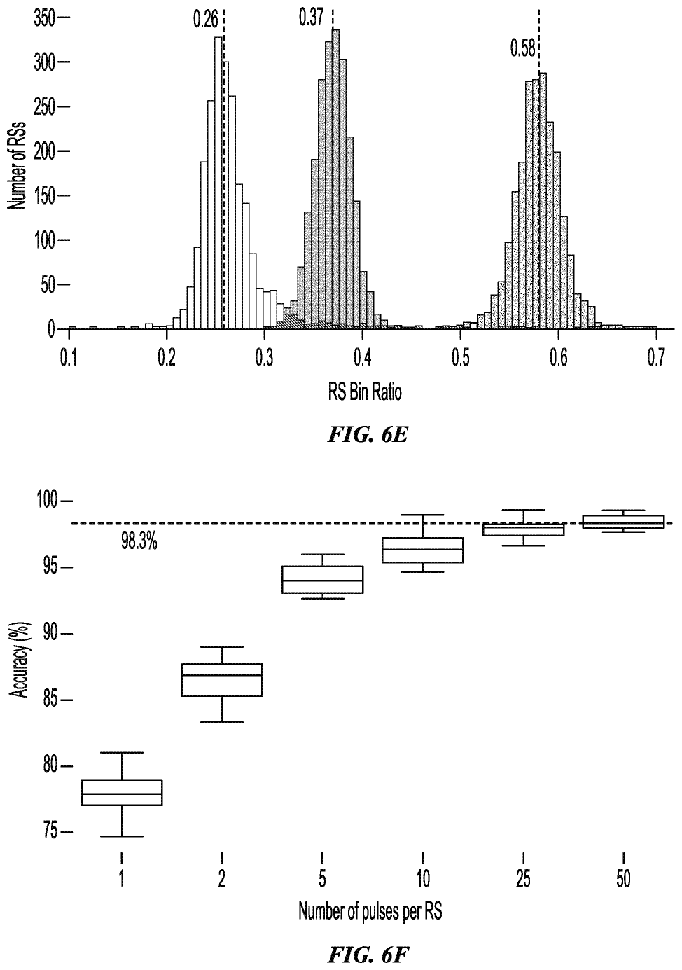

Here, a novel single-molecule protein sequencing approach and integrated system for massively parallel proteomic studies was demonstrated. In this approach, peptides are immobilized in nanoscale reaction chambers on a semiconductor chip and N-terminal amino acids (NAAs) with dye-labeled NAA recognizers are detected in real time. Aminopeptidases sequentially remove individual NAAs to expose subsequent amino acids for recognition, eliminating the need for complex chemistry and fluidics (FIG. 1). A benchtop device with a 532 nm pulsed laser source for fluorescence excitation and electronics for signal processing was built (FIG. 6A). The semiconductor chip uses intensity and fluorescence lifetime, rather than emission wavelength, for discrimination of dye labels. The recognizers detect one or more types of NAAs and provide information for peptide identification based on the temporal order of NAA recognition and the kinetics of on-off binding.

CMOS fabrication technology was used to build a custom time-domain-sensitive semiconductor chip with nanosecond precision, containing fully-integrated components for single-molecule detection, including photosensors, optical waveguide circuitry, and reaction chambers for biomolecule immobilization (FIG. 1). Observation volumes less than 5 attoliters was achieved through evanescent illumination at reaction chamber bottoms from the nearby waveguide, enabling sensitive single-molecule detection in the context of high freely-diffusing dye concentrations (>1 μM).

The semiconductor chip uses a novel filterless system that excludes excitation light on the basis of photon arrival time, achieving greater than 10,000-fold attenuation of incident excitation light. Elimination of the need for an integrated optical filter layer increases the efficiency of fluorescence collection and enables scalable manufacturing of the chip. To enable discrimination of fluorescent dye labels attached to NAA recognizers by fluorescence lifetime and intensity, the chip rapidly alternates between early and late signal collection windows associated with each laser pulse, thereby collecting different portions of the exponential fluorescence lifetime decay curve. The relative signal in these collection windows (termed “bin ratio”) provides a reliable indication of fluorescence lifetime (FIGS. 6B-F, and Materials and Methods).

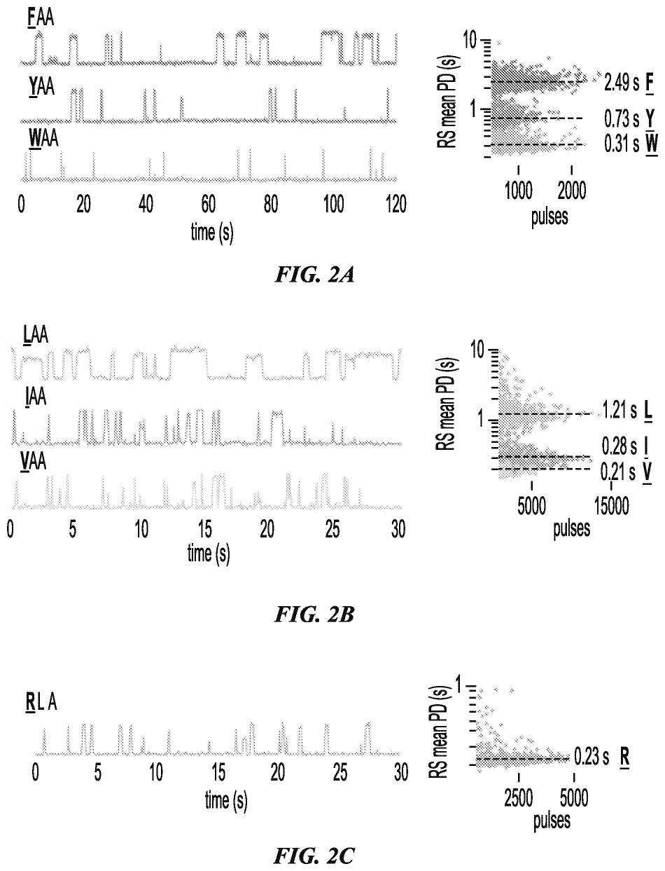

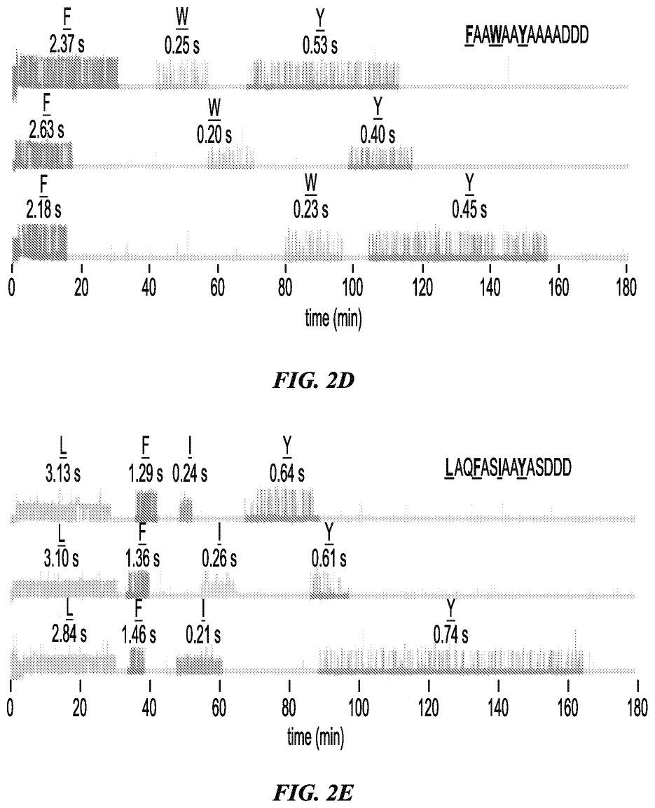

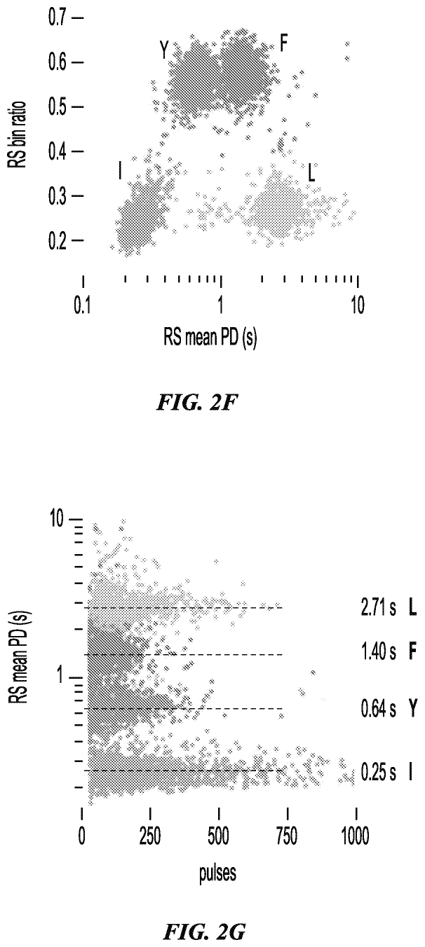

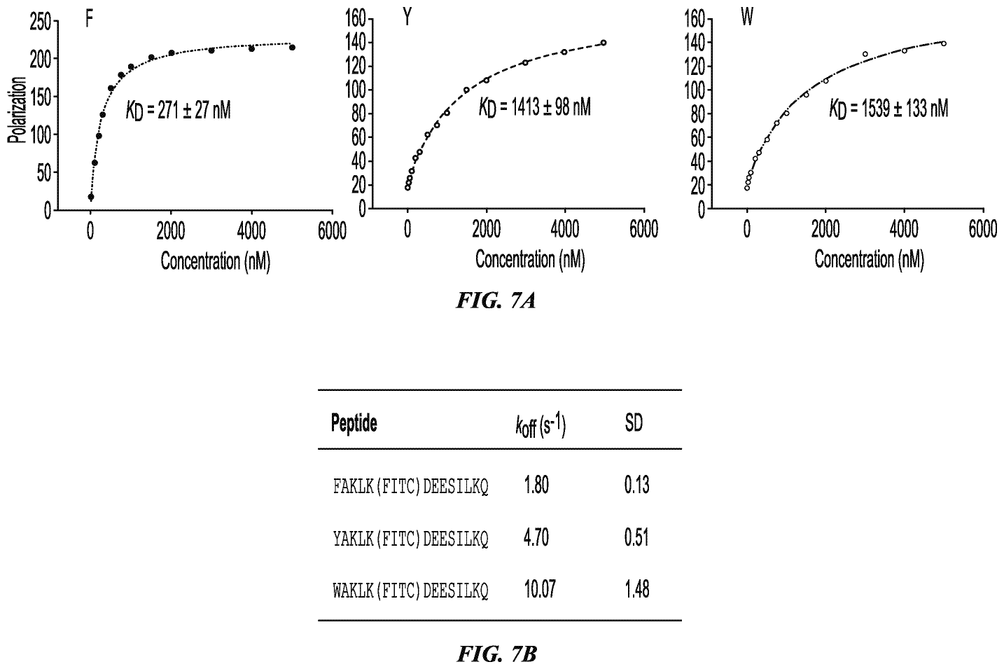

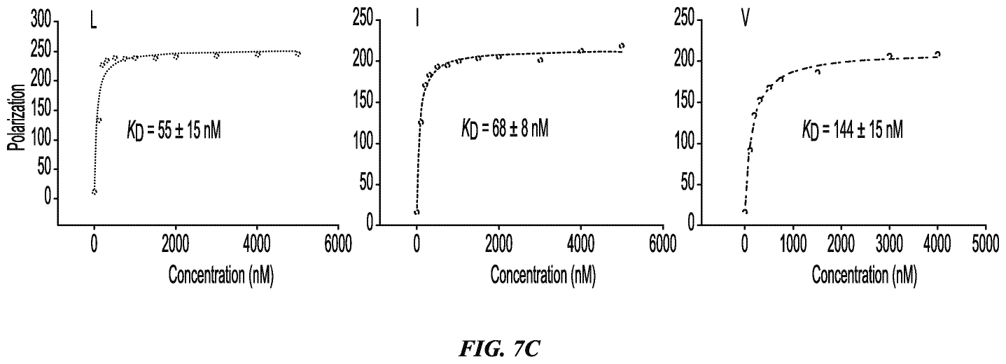

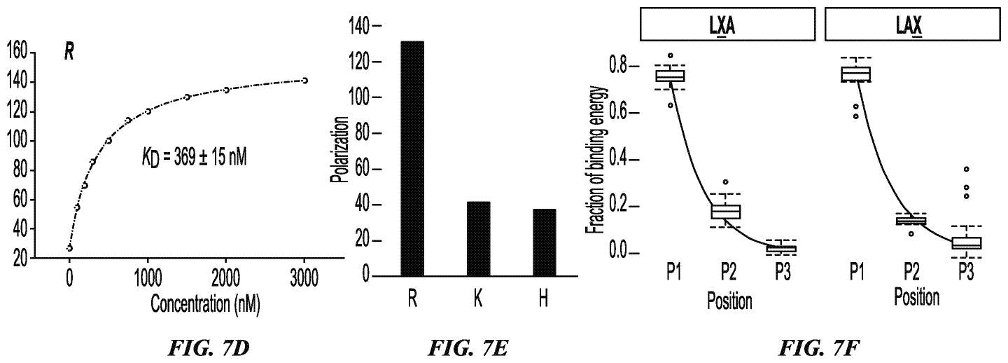

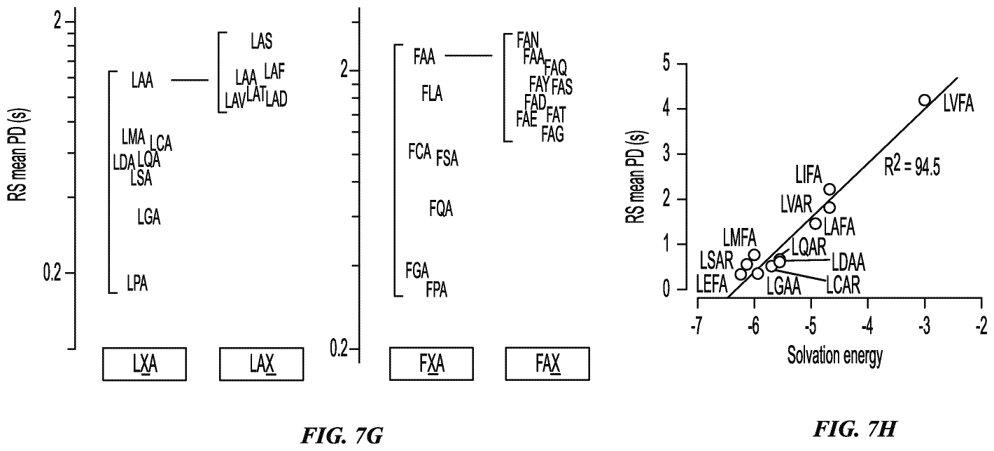

In order for NAA binding proteins to function as recognizers in this approach, the average lifetime of the bound recognizer-peptide complex must be long enough (typically >120 ms) to generate detectable single-molecule binding events. Proteins from the N-end rule adapter family ClpS that natively bind to N-terminal phenylalanine, tyrosine, and tryptophan were evaluated. Using PS610, a recognizer derived from ClpS2 from A. tumefaciens, it was established that this recognizer binds detectably to immobilized peptides with these NAAs. Importantly, it was also determined that the kinetics of binding differ for each NAA. To demonstrate these properties, immobilized peptides containing the initial N-terminal sequences FAA, YAA, or WAA were incubated on separate chips with PS610 and data collected for 10 hours (Methods). NAA recognition was observed by PS610, characterized by continuous on-off binding during the incubation period, with distinct pulse duration (PD) for each peptide (FIG. 2A). Median PDs were 2.51, 0.73, and 0.31 s for FAA, YAA, and WAA, respectively. These values reflect differences in binding affinity driven by different dissociation rates for each type of protein-NAA interaction⁷ (FIGS. 7A-B).

To expand the set of recognizable NAAs, N-end rule pathway proteins were investigated as a source of additional recognizers. In a comprehensive screen of diverse ClpS family proteins, a novel group of ClpS proteins from the bacterial phylum Planctomycetes with native binding to N-terminal leucine, isoleucine, and valine was discovered. Directed evolution techniques were applied to generate a Planctomycetes ClpS variant-PS961—with sub-micromolar affinity to N-terminal leucine, isoleucine, and valine, and recognition of these NAAs was demonstrated (FIG. 2B). The median PD of binding to peptides with N-terminal LAA, IAA, and VAA was 1.21, 0.28, and 0.21 s, respectively, in agreement with bulk characterization (FIG. 7C).

In a separate screen, a diverse set of UBR-box domains from the UBR family of ubiquitin ligases that natively bind N-terminal arginine, lysine, and histidine were investigated. The UBR-box domain from the yeast K. lactis UBR1 protein exhibited the highest affinity for N-terminal arginine, and this protein was used to generate an arginine recognizer, PS691. PS691 recognized arginine in a peptide with N-terminal RLA with a median PD of 0.23 s (FIG. 2C). Lower affinity binding to N-terminal lysine and histidine (FIGS. 7D-E) was insufficient for single-molecule detection.

To demonstrate that amino acids in a single peptide molecule can be sequentially exposed by aminopeptidases and recognized in real time with distinguishable kinetics, an immobilized peptide containing the initial sequence FAAWAAYAA (SEQ ID NO: 1073) was incubated with PS610 for 15 minutes, followed by addition of PhTET3, an aminopeptidase from P. horikoshii. The collected traces consisted of regions of distinct pulsing, which were referred to as recognition segments (RSs), separated by regions lacking recognition pulsing (non-recognition segments, NRSs). Analysis software was developed to automatically identify pulsing regions and transition points within traces (Methods). Traces began with recognition of phenylalanine with median PD of 2.36 s (FIG. 2D), in agreement with the PD observed for FAA in recognition-only assays. This pattern terminated after aminopeptidase addition (on average 11 min after addition), and was followed by the ordered appearance of two RSs with median PD of 0.25 s and 0.49 s (FIG. 2D), corresponding to the short and medium PDs obtained in YAA and WAA recognition-only assays. Thus, the introduction of aminopeptidase activity to the reaction resulted in the sequential appearance of discrete RSs with the expected kinetic properties in the correct order.

To demonstrate dynamic sequencing with two NAA recognizers, PS610 and PS961 were labeled with the distinguishable dyes atto-Rho6G and Cy3, respectively, and an immobilized peptide of sequence LAQFASIAAYASDDD (SEQ ID NO: 1035) was exposed to a solution containing both recognizers. After 15 minutes, two P. horikoshii aminopeptidases with complementary activity covering all 20 amino acids were added-PhTET2 and PhTET3. The collected traces displayed discrete segments of pulsing alternating between PS961 and PS610 according to the order of recognizable amino acids in the peptide sequence (FIG. 2E). The average bin ratio and average PD associated with each RS readily distinguished the two dye labels and four types of recognized NAAs (FIG. 2F). Median PDs were 2.70, 1.43, 0.25, and 0.66 s for N-terminal LAQ, FAS, IAA, and YAS, respectively (FIG. 2G).

Previous studies have shown that NAA-bound ClpS and UBR proteins also make contacts with the residues at position 2 (P2) and position 3 (P3) from the N-terminus that influence binding affinity. These influences are reflected in the modulation of PD depending on the downstream P2 and P3 residues, as observed above for LAA (1.21 s) compared to LAQ (2.70 s). It was found that these influences on PD vary within informatically advantageous ranges and can be determined empirically or approximated in silico to model peptide sequencing behavior a priori (FIGS. 7F-H). A powerful feature of this recognition behavior in regards to peptide identification is that each RS contains information about potential downstream P2 and P3 residues or PTMs, whether or not these positions are the targets of an NAA recognizer.

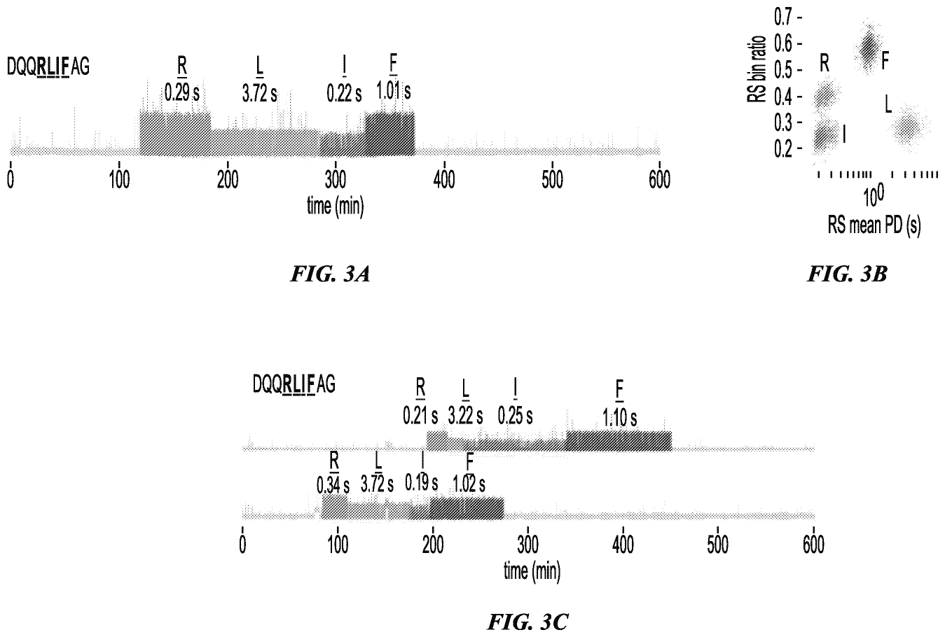

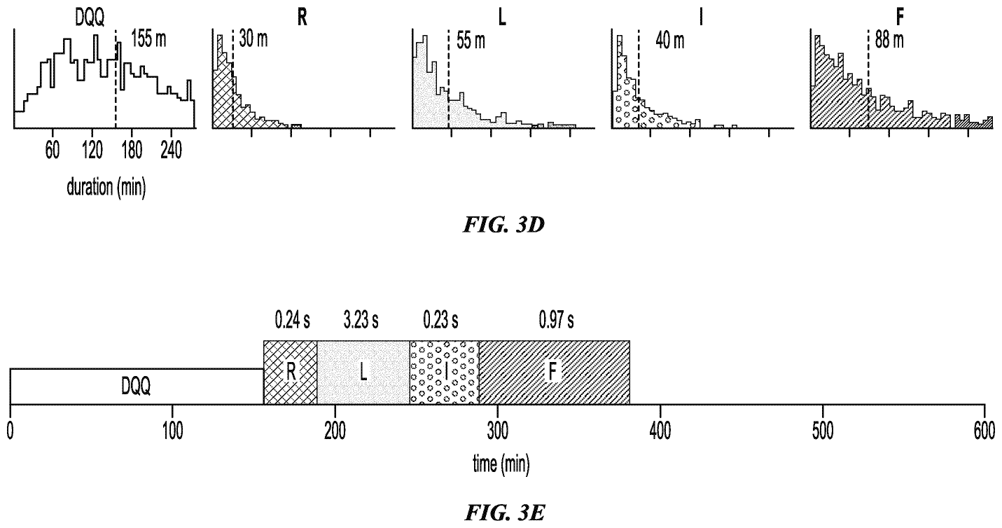

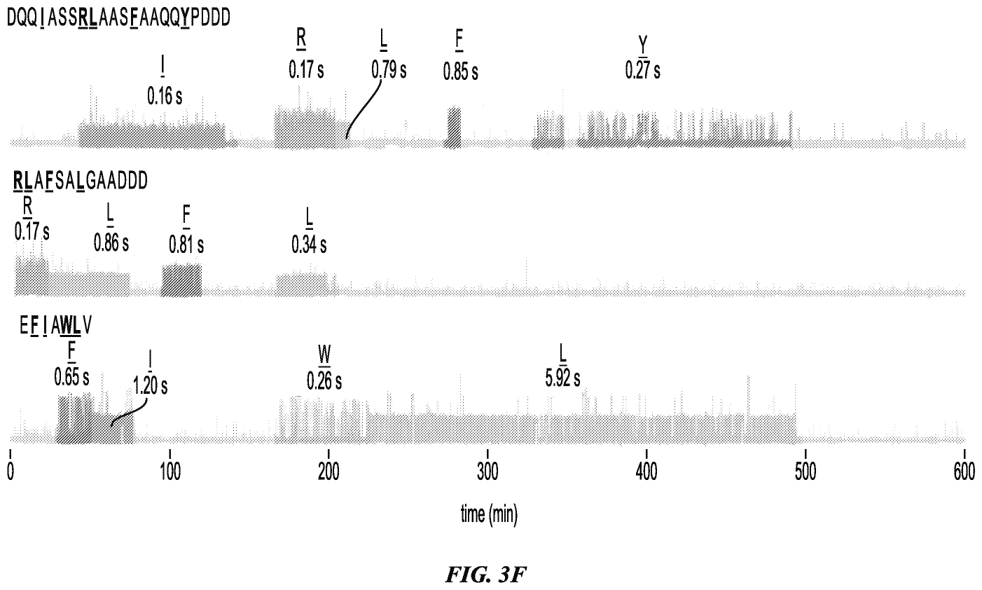

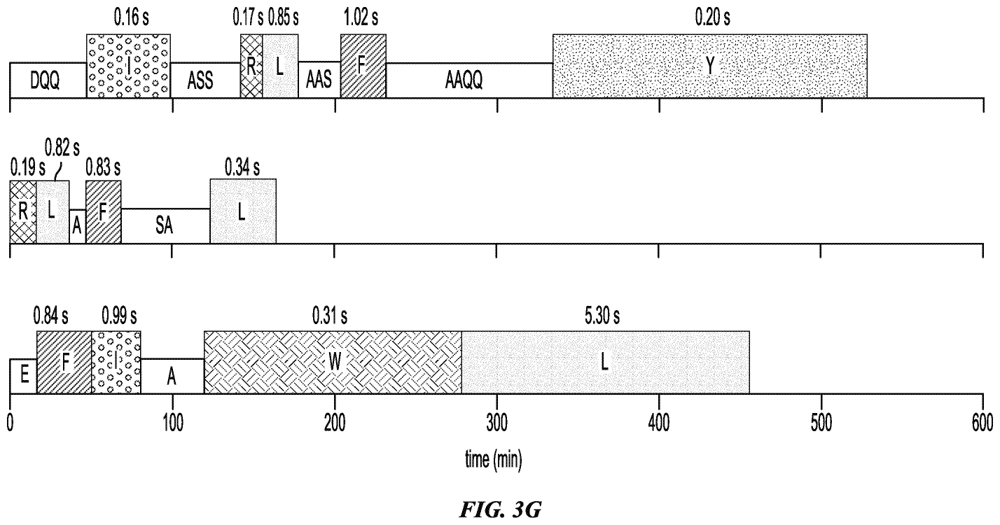

To evaluate the kinetic principles of the dynamic sequencing method when applied to diverse sequences, the synthetic peptide DQQRLIFAG (SEQ ID NO: 1036), corresponding to a segment of human ubiquitin was first characterized (FIGS. 3A-D). Sequencing reactions were performed using a combination of three differentially-labeled recognizers—PS610, PS961, and PS691—and two aminopeptidases—PhTET2 and PhTET3 (Materials and Methods). The example trace in FIG. 3A starts with an NRS that corresponds to the time interval during which residues in the initial DQQ motif are present at the N-terminus. The first RS starts at 120 min, upon exposure of N-terminal arginine to recognition by PS691. Subsequent cleavage events sequentially expose N-terminal leucine, isoleucine, and phenylalanine to their corresponding recognizers, with fast transitions (average <10 s) from one RS to the next. The transition from leucine to isoleucine recognition by PS961 is readily identified as a sharp change in average PD. This overall pattern is replicated across many instances of sequencing of the same peptide, with similar PD statistics across traces, as each peptide molecule follows the same reaction pathway over the course of the sequencing run (FIGS. 3B-C). Due to the stochastic timing of cleavage events, each trace displays distinct start times and durations for each RS (FIG. 3C).

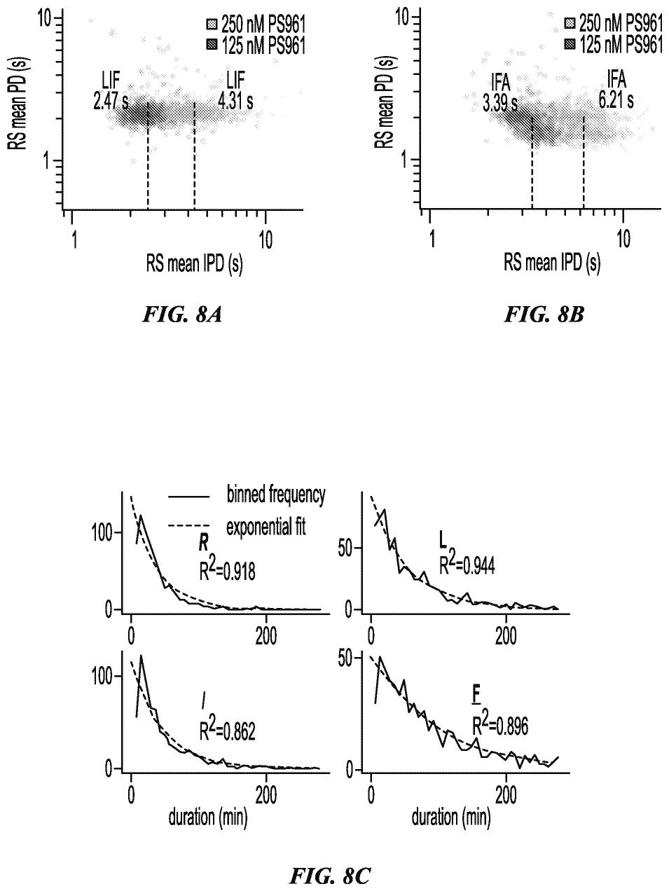

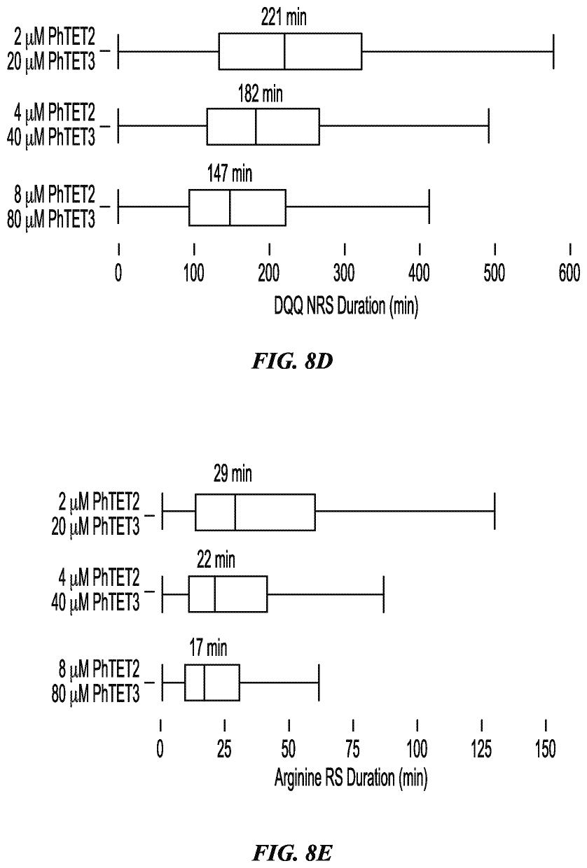

This approach reports the binding kinetics at each recognizable amino acid position and the kinetics of aminopeptidase cleavage along the peptide sequence. High-precision kinetic information on binding is obtained from a single trace, since each RS typically contains tens to hundreds of on-off binding events, resulting in a distribution of PD and interpulse duration (IPD) measurements that can be analyzed statistically. The repetitive probing of each NAA also provides accurate recognizer calling, since calls are not based on the error-prone detection of a single event associated with one fluorophore molecule (FIG. 6F). Recognizer concentration governs IPD for each RS; higher recognizer concentrations result in shorter average IPDs and faster rates of pulsing (FIGS. 8A-B). Higher recognizer concentrations, however, increase the fluorescence background from freely diffusing recognizers, resulting in lower pulse signal-to-noise, and can compete with aminopeptidases for N-terminal access. In practice, IPDs in the range of approximately 2 to 10 s provide a favorable balance among these factors.

The distribution of RS durations across an ensemble of replicate traces defines the rate of cleavage of each recognizable NAA. For DQQRLIFAG (SEQ ID NO: 1036) peptide, average cleavage times of 31, 54, 39, and 86 min were observed for N-terminal arginine, leucine, isoleucine, and phenylalanine, respectively, with approximate single-exponential decay statistics for each position (FIG. 3D, FIG. 8C). The distribution of NRS durations reports the cleavage rate of a run of one or more non-recognized NAAs. The average NRS duration for the initial DQQ motif was 153 min (FIG. 3D). Average cleavage rates are a key parameter and are controlled by the aminopeptidase concentration in the assay (FIGS. 8D-E). Given the exponential behavior, average RS durations of 10 to 40 min were targeted to provide sufficient time for pulsing data collection, avoid missed RSs due to rapid cleavage, and minimize excessively long RS durations. It was found helpful to visualize the sequencing profiles of peptides as kinetic signature plots-simplified trace-like representations of the time course of complete peptide sequencing containing the median PD for each RS, and the average duration of each RS and NRS (FIG. 3E). These highly characteristic features provide a wealth of sequence-dependent information for mapping traces from peptides to their proteins of origin.

To demonstrate that this core methodology and its kinetic principles apply to a wide range of peptide sequences, the synthetic peptides DQQIASSRLAASFAAQQYPDDD (SEQ ID NO: 1037), RLAFSALGAADDD (SEQ ID NO: 1038), and EFIAWLV (SEQ ID NO: 1039) (a segment of human GLP-1) were sequenced under the same sequencing conditions used for DQQRLIFAG (SEQ ID NO: 1036) (FIG. 3F). Each peptide generated a characteristic kinetic signature in accordance with its sequence (FIG. 3G). Readouts as far as position 18 (the furthest recognizable amino acid) in the peptide DQQIASSRLAASFAAQQYPDDD (SEQ ID NO: 1037) were obtained, illustrating that the method is compatible with long peptides and capable of deep access to sequence information in peptides of lengths found in typical protein digests.

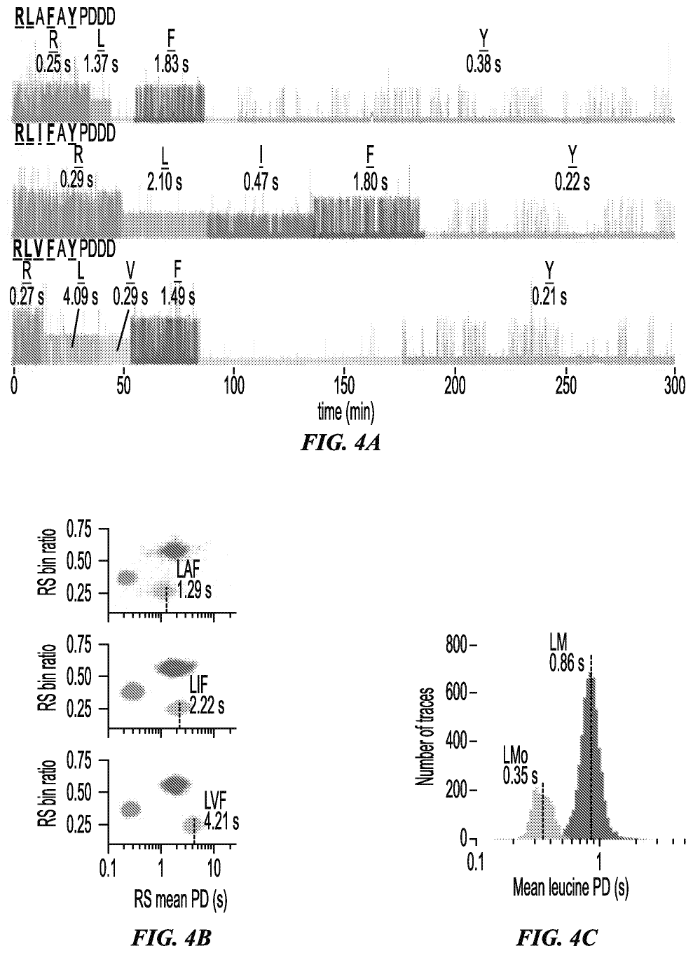

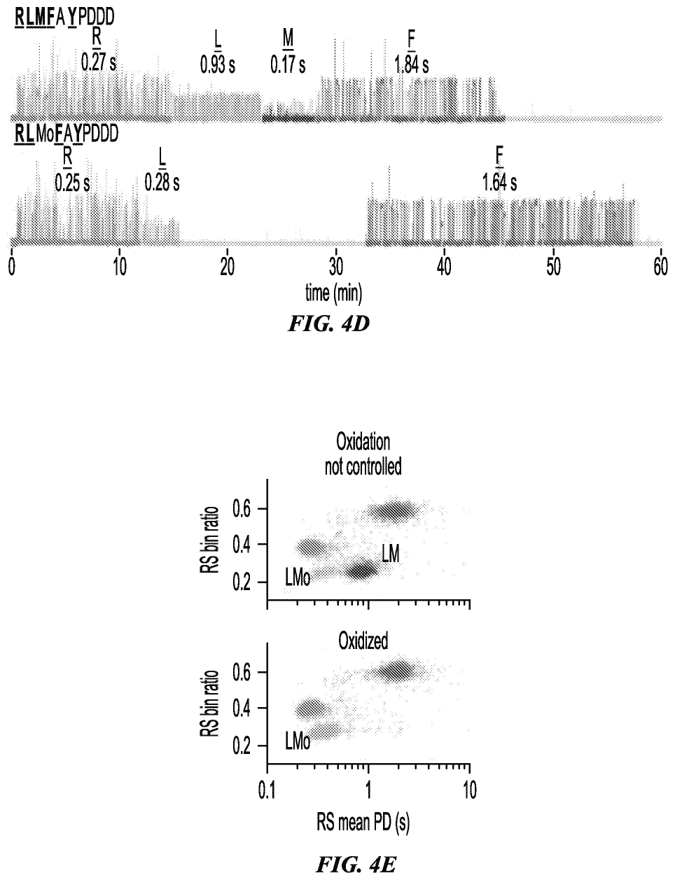

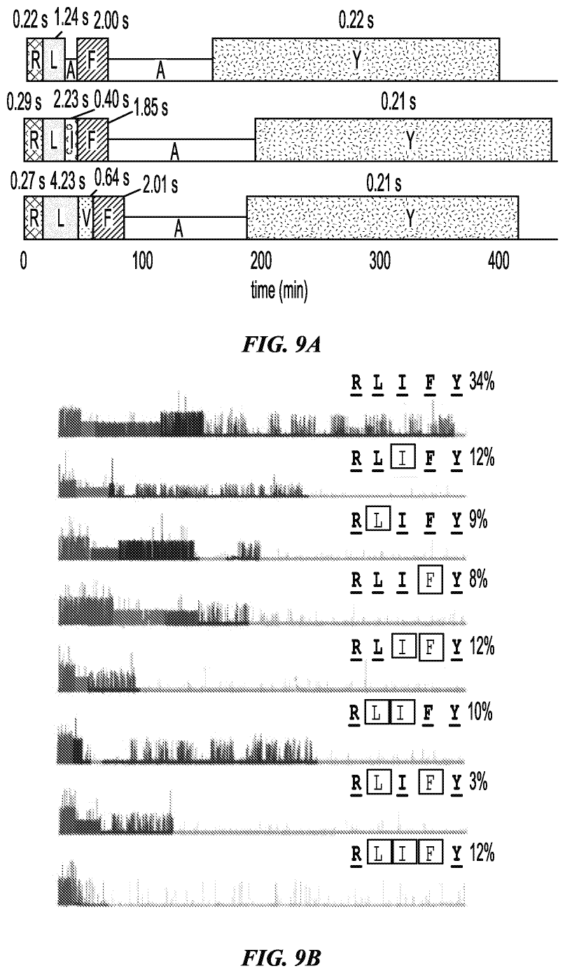

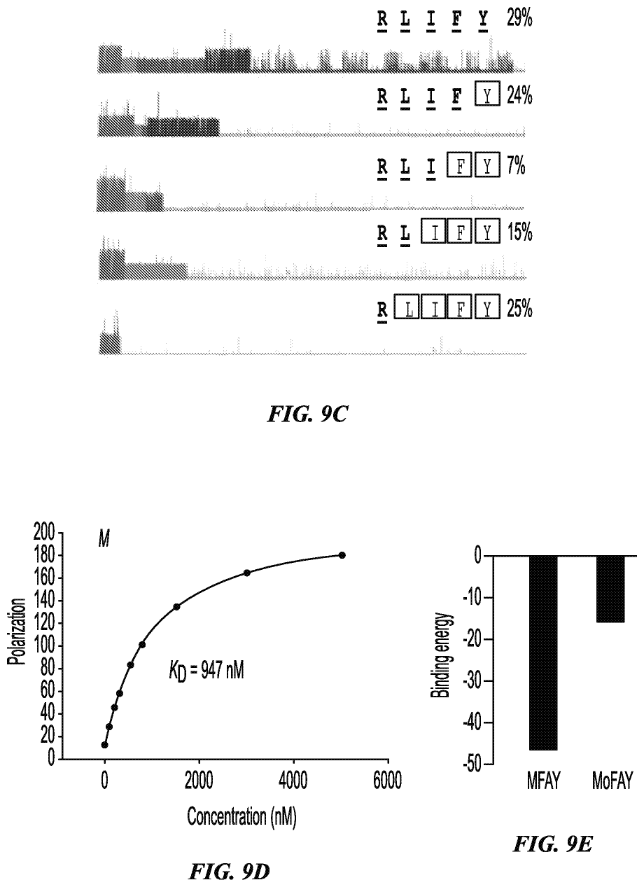

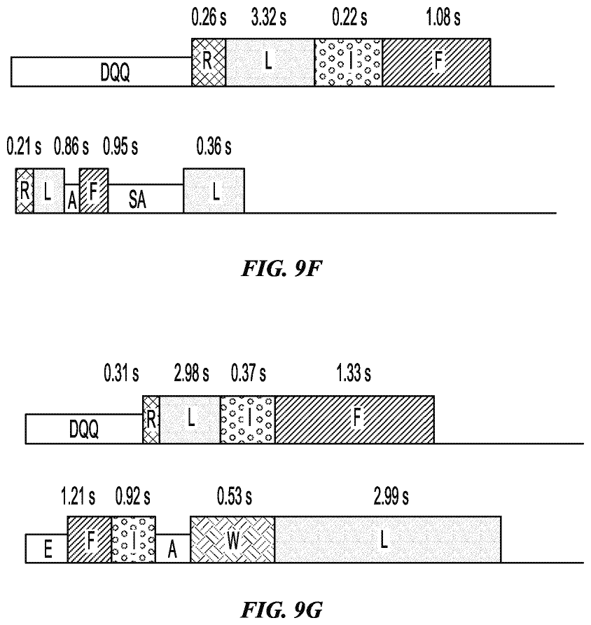

To illustrate how the kinetic parameters acquired from sequencing are sensitive to changes in sequence composition, sequencing was performed with a set of three peptides-RLAFAYPDDD (SEQ ID NO: 1040), RLIFAYPDDD (SEQ ID NO: 1041), and RLVFAYPDDD (SEQ ID NO: 1042)—that differ only at a single position, located immediately downstream from the PS961 N-terminal target leucine. Each type of amino acid at this position had a distinct effect on the PD acquired during recognition of N-terminal leucine by PS961. Median PDs of 1.29 s, 2.22 s, and 4.21 s were observed for LAF, LIF, and LVF, respectively (FIG. 4B). In addition to differences in PD for leucine, each peptide displayed a characteristic RS or NRS in the interval between leucine and phenylalanine recognition (FIG. 4A, FIG. 9A). These results demonstrate the sensitivity of the sequencing readout to variation at a single position and illustrate that both directly recognized NAAs and adjacent residues can influence the full kinetic signature obtained from sequencing.

Since the aminoacyl-proline bond of the YP motif in peptides such as RLIFAYPDDD (SEQ ID NO: 1041) cannot be cleaved by the PhTET aminopeptidases, observation of YP pulsing at the end of a trace ensures that cleavage has progressed completely from the first to last recognizable amino acid. The sequencing output from RLIFAYPDDD (SEQ ID NO: 1041), therefore, provided a convenient dataset for examining biochemical sources of non-ideal behavior that could lead to errors in peptide identification. The main sources of incomplete information in traces were deletions of expected RSs due to the stochastic occurrence of rapid sequential cleavage events (FIG. 9B) and early termination of reads resulting from photodamage or surface detachment (FIG. 9C).

In addition to changes in amino acid sequence composition, sequencing readouts are sensitive to changes due to PTMs. As an example, methionine oxidation was examined. The thioether moiety of the methionine side chain is susceptible to oxidation during peptide synthesis and sequencing. It was determined that PS961 binds a peptide with N-terminal methionine with a K_{D} of 947 nM (FIG. 9D) and it was hypothesized that oxidation, resulting in a polar methionine sulfoxide side chain, would eliminate binding and reduce NAA binding affinity when located at P2. It was determined computationally that methionine sulfoxide is highly unfavorable in the PS961 NAA binding pocket and that non-polar residues are preferred at P2 (FIG. 9E). The synthetic peptide RLMFAYPDDD (SEQ ID NO: 1043) was sequenced, and two populations of traces with distinct kinetic signatures were observed—a first population containing leucine recognition with median PD of 0.86 s, and a second population with median PD of 0.35 s (FIG. 4C). Traces from the first population also displayed methionine recognition with short PD in the time interval between leucine and phenylalanine recognition (FIG. 4E). Methionine recognition was absent in traces from the second population (FIG. 4D), indicating that the methionine side chain in these peptides was not capable of recognition by PS961. When methionine was fully oxidized by preincubation with hydrogen peroxide (Materials and Methods), elimination of both methionine recognition and of the leucine recognition cluster was observed with long median PD, as expected (FIG. 4E). These results demonstrate the capability for extremely sensitive detection of PTMs due to their kinetic effects on recognition.

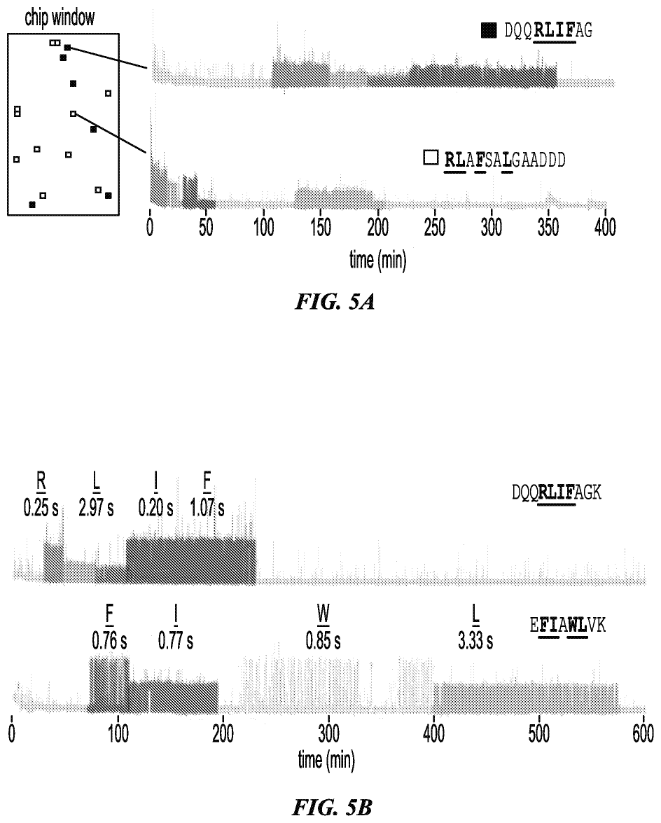

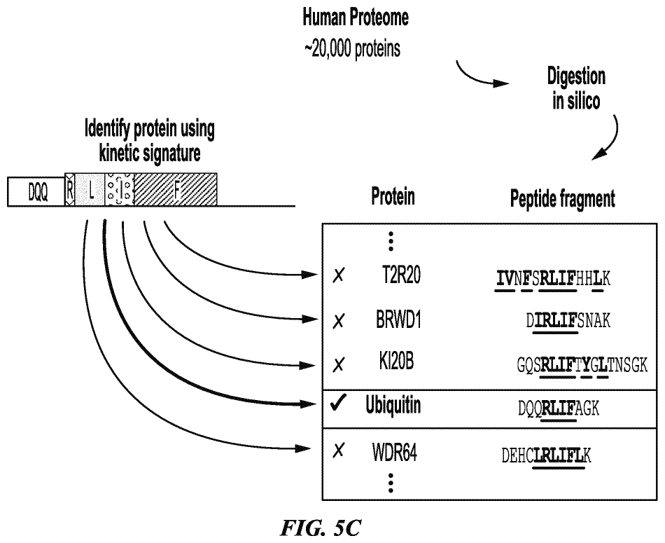

Proteomics applications require identification of peptides in mixtures derived from biological sources. To extend the results to peptide mixtures and biologically-derived peptides, two experiments were performed. First, DQQRLIFAG (SEQ ID NO: 1036) and RLAFSALGAADDD (SEQ ID NO: 1038) peptides were mixed, immobilized on the same chip, and a sequencing run was performed. Data analysis (Materials and Methods) identified two populations of traces corresponding to each peptide, with kinetic signatures in close agreement with those identified in runs with individual peptides (FIG. 5A, FIG. 9F). Second, to demonstrate that the method extends to biologically derived peptides, sequencing runs were performed with peptide libraries generated using a simple workflow from recombinant human ubiquitin (76 amino acids) and GLP-1 (37 amino acids) proteins digested with AspN/LysC and trypsin, respectively (Methods). For both libraries, data analysis readily identified traces matching the expected recognition pattern for the protease cleavage products DQQRLIFAGK (SEQ ID NO: 1045) and EFIAWLVK (SEQ ID NO: 1046) for ubiquitin and GLP-1, respectively, and produced kinetic signatures in agreement with synthetic versions of these peptides (FIG. 5B, FIG. 9G). Matches to the kinetic signature of the ubiquitin peptide DQQRLIFAGK (SEQ ID NO: 1045) were identified across the human proteome, taking advantage of simple sequence constraints provided by kinetic information (Materials and Methods). Only one protein other than ubiquitin was found containing a peptide that could potentially match this signature; thus even short signatures can exhibit proteome abundance of less than one in 10⁴ proteins. These results illustrate the potential of the full kinetic output from sequencing to enable digital mapping of peptides to their proteins of origin.

### DISCUSSION

The simple, real-time dynamic approach differs markedly from other recently described single-molecule approaches that rely on complex, iterative methods involving stepwise Edman chemistry or hundreds of cycles of epitope probing; nanopore approaches offer the potential for real-time readouts and simplicity, but face substantial challenges related to the size and biophysical complexity of polypeptides. The sequencing technology described herein is readily expanded in its capabilities, and there are multiple areas for improvement. Expansion of proteome coverage can be achieved through directed evolution and engineering of recognizers. The NAA targets demonstrated here comprise approximately 35.6% of the human proteome, but lower-affinity NAA targets require longer PD to enable detection in all sequence contexts.

Recognizers for new amino acids or PTMs can be evolved from current recognizers or identified in screens of other scaffolds, such as other types of NAA- or PTM-binding proteins or aptamers. Overall, scaling to detection of all 20 natural amino acids and multiple PTMs is feasible for de novo sequencing; however partial sequences are sufficient for most proteomics applications, which rely on mapping to pre-defined sets of candidate proteins. Aminopeptidases can be engineered to optimize cleavage rates and minimize RS deletions from rapid sequential cleavage. It is envisioned that the dynamic range of samples and the applications most suitable for the system will tend to scale with the number of reaction chambers on the chip, and that compression of dynamic range will be necessary for certain applications.

It is anticipated that the sequencing technology demonstrated here will increase the accessibility of proteomics studies, enable new discoveries in biological and clinical research, and help power a new generation of precision medicine.

### Materials and Methods

### Semiconductor Device Operation and Bin Ratio Calculation

Experiments were performed on pre-production semiconductor chips with 296K active wells, taking into account some loss to flow cell occlusion of the sensor array. A dual chamber flow cell allows for two independent samples to be sequenced in parallel, each utilizing 148K active wells. Initial production devices have 2M active wells, scaling to tens of millions of active wells using standard CMOS processing for the first product line. Pulsed 532 nm excitation light from a 67 MHz mode-locked laser is coupled into a grating coupler at the edge of the semiconductor chip. The use of a single laser wavelength—in combination with fluorescent dye discrimination by fluorescence intensity and lifetime—reduces size, cost, and complexity, contributing to the scalability of the platform. A network of optical waveguides divides the excitation light and routes it to the sensor array to illuminate each reaction chamber. Each CMOS pixel contains a single light-sensitive photodiode with two high-speed global shutters (a reject gate and a collect gate) that discard and collect photoelectrons (chip photonic structures reduce pixel-to-pixel crosstalk to less than 2%). Control waveforms are applied to the collect and reject gates synchronously with the incident pulsed light source (FIG. 6B). Approximately 1 ns before the excitation pulse, the reject gate is charged to >3 volts and the collect gate is discharged below 1 volt. Scattered 532 nm excitation photons generate photoelectrons in the photodiode. The photoelectrons are quickly transferred to a high voltage drain by built-in potential fields within the photodiode and the reject gate potential. Between 1 and 3 ns after excitation, the collect gate is charged to >3 volts, the reject gate is discharged to <1 volt. Photoelectrons generated from emitted photons that arrive in the photodiode after the collect gate is opened are transferred to a storage node within each pixel. Photoelectrons are accumulated for 7.5 to 30 ms, configurable, within each pixel across approximately 500,000 to 2,000,000 laser pulses (FIG. 6B). The accumulated charge in the storage node is measured with the standard transfer gate, floating diffusion, source follower, row select, and on-chip analog-to-digital converters common to all CMOS image sensors, enabling scaling to large array sizes with small pixels. Fluorescence lifetime information is obtained by alternating the timing of the collect and reject gate waveforms between subsequent measurements. In the first measurement, only emission photoelectrons that arrive >3 ns after the excitation pulse (bin 0) are collected. In the second measurement, emission photoelectrons that arrive >Ins after the excitation pulse (bin 1) are collected. Signal measured from the pixel as the phase relationship between the excitation source and the gate waveforms is adjusted throughout the entire excitation cycle demonstrates the pixel transitioning from 100% collection of photons during the collect phase to extinction of greater than 99.99% of photons during the rejection phase in less than Ins (FIG. 6C). The ratio of these two measurements (bin ratio) provides an estimate of the fluorescence lifetime (FIG. 6D). We have demonstrated the ability to differentiate multiple dyes based on bin ratio alone (FIG. 6E).

### Peptide Synthesis and Labeling

Peptides were synthesized on Rink Amide Resin on a PurePrep Chorus Solid-phase peptide synthesizer (Gyros Protein Technology) using Standard Fmoc chemistry. All synthetic peptides contained C-terminal Fmoc-azidolysine. The resin was deprotected in a mixture of TFA/TIPS/H2O (2.5%/2.5%/95%) at room temperature for 1.5 h. The deprotection mixture was concentrated under an argon stream. The peptides were precipitated from cold diethyl ether, resuspended in 1:1 water-acetonitrile, and purified on reverse phase HPLC (X-bridge C18, Waters) with a gradient of 10-70% acetonitrile (0.05% TFA) over 20 min. The residue was dried under high vacuum to generate white pellets. Into a solution of DBCO-DNA-biotin (2 nmol in 100 uL PBS) was added the peptide stock solution (4 uL, 5 mM) at room temperature. The reaction progress was monitored on LC-MS (Thermo UltiMate 3000 Executive Plus). After the reaction was completed, the mixture was conjugated to an excess of streptavidin. The peptide-DNA-streptavidin complex was purified on an ion exchange HPLC (DNAPac 200, Thermo). Gradient, buffer A, 20 mM sodium phosphate buffer, pH 8.5, buffer B, 1 M NaBr, 20 mM sodium phosphate buffer, pH 8.5, 20-60% B over 15 min. The purified complex was buffer-exchanged to a solution containing 50 mM MOPS (pH 8.0) and 60 mM potassium acetate on a 30K MWCO spin filter before use. The peptide containing fully oxidized methionine was prepared by mixing 3% hydrogen peroxide with the methionine peptide in 1:1 water-methanol at room temperature for 20 minutes. The product was immediately purified on a reverse-phase HPLC using the same peptide purification method described above, the purity was verified by reverse-phase HPLC (Thermo UltiMate 3000) on an analytical column (Zorbax SB-Aq, 5 μm, 4.6×250 mm), and the correct mass of the oxidized product was verified by LC-MS (Agilent LC-MSD-iQ, positive mode).

### Protein Digestion and Labeling

GLP-1 7-37, GLP-2, and Ubiquitin (1-76) recombinant proteins were purchased from RnD Systems as lyophilized powder. Each protein was reconstituted in 100 mM HEPES, pH 8.0 (20% acetonitrile) to a final concentration of 200 μM. When necessary, cysteines were reduced and alkylated using TCEP (2 mM) and iodoacetamide (10 mM). GLP1 and GLP2 were digested using 1 μg of Trypsin (LCMS grade, Pierce) at 37° C. overnight. Ubiquitin was digested using 1 μg of LysC (LCMS grade, Pierce) and 1 μg of rAspN (LCMS grade, Promega). After protease digestion, pH of peptide mixtures was adjusted to pH 10.5 using potassium carbonate (57 mM), and lysines were converted to azidolysines using imidazole-1-sulfonyl azide (ISA, 2 mM) and copper sulfate catalyst (0.5 mM). ISA was quenched using polyurethane beads bearing an amine functionality (Oligo Factory). The mixture was then filtered and adjusted to pH 7-8 using 1 M acetic acid. The solution was diluted in 50% (v/v) of 10 mM MOPS, 10 mM KOAc, pH 7.5, added to DNA-streptavidin-DBCO complex, and incubated at 37° C. for 12-16 h. When required, the detergent Cetrimonium bromide was added to the reaction at a final concentration of 0.25 mM.

### Recognizer Purification, Labeling, and Characterization

Expression vectors (with pET30 a+ backbone) for recognizers and Biotin ligase were co-transformed into BL21(DE3) chemically competent E. coli cells. The transformed cells were plated on Luria agar plates containing carbenicillin (50 μg/mL) and kanamycin (25 μg/mL) and incubated overnight at 37° C. to obtain single colonies. The starter liquid cultures inoculated with colonies were grown in Luria broth with ampicillin (50 μg/mL) and kanamycin (25 μg/mL) and inoculated into large cultures at a starting optical density (OD600) of ˜0.01. The expression cultures were incubated at 37° C. at 230 rpm until OD600 approached ˜0.7. The cultures were then induced with 4 mM IPTG. The expressed recognizer was biotinylated in vivo by adding 8 mM biotin at the same time as IPTG. Cells were harvested after ˜12 hrs of expression by centrifugation at 10,000 g at 4° C., and the cell pellets were washed with 1×PBS buffer pH 7.4. The cells were resuspended in Bug buster HT (Thermo Fisher Scientific) and incubated at room temperature for 30 mins on a magnetic stirrer. The cell suspension was then diluted with equal volume of 2× lysis buffer (100 mM Tris-HCl pH 7.5, 10% glycerol, 0.5 M NaCl) and incubated at room temperature for 30 mins on a magnetic stirrer. The lysate was centrifuged at 21,000 g at 4° C. to remove cell debris. Supernatant was collected and loaded on a Nickel NTA resin (Cytiva) affinity column pre-equilibrated with Buffer A (50 mM Tris-HCl pH 7.5, 10% glycerol, 0.5 M NaCl) on an AKTA Pure (Cytiva) system. The column was washed with at least ten column volumes of the buffer containing 10 mM imidazole. Elution was performed using a 10-300 mM imidazole gradient. Eluted fractions were dialyzed in a 10 kDa cassette against 4 L of dialysis buffer (50 mM Tris-HCl pH 7.5, 0.2 M NaCl, 50% glycerol) at 4° C. overnight.

For labeling of the recognizers, equal volumes of recognizer and DNA-Dye-Streptavidin complex were mixed at 5:1 (recognizer:DNA-dye-SV) molar ratio. The mixture was incubated on ice for 30 m and dialyzed overnight against SEC buffer (25 mM HEPES pH 8.0, 150 mM KCl). The recognizer-dye conjugate was harvested from the dialysis and centrifuged at 10,000 g at 4° C. Supernatant was collected and concentrated using 10 kDa cut off concentrators. The concentrated conjugate was purified on an Agilent 1260 Infinity HPLC system using a size exclusion column (BioSEC-3 300 Å, 3 μm).

Binding affinity was measured by polarization using a labeled peptide. The polarization response and total intensity measurements were carried out at 20° C. on a microplate fluorometer with 480 nm excitation and 530 nm emission. The interaction of recognizer with labeled peptide containing a target N-terminal residue (XAKLDEESILKQK-FITC (SEQ ID NO: 1074)) was performed in PBS buffer at pH 7.4 and readings were collected after 30 min. Multiple analyses were performed at increasing recognizer concentration at a fixed concentration of a target peptide to obtain a titration curve. An equilibrium polarization response at each concentration was plotted and fit to calculate the K_{D}.

The off-rate (k_{off}) of PS610 was measured for various peptides using a stopped flow instrument. Labeled peptide (50 nM) was mixed with PS610 in PBS buffer pH 7.4 with 0.01% Tween-20 and incubated at 30° C. After 30 min of incubation, the recognizer:peptide complex was rapidly mixed with 10-20 fold molar excess of unlabeled trap peptide and the reaction was followed in real time by measuring the fluorescence intensity. At least three-time course traces were averaged and fit to an exponential equation.

### Aminopeptidase Purification

Expression vectors (with pET30 a+ backbone) for aminopeptidases PhTET2 and PhTET3 were transformed into BL21(DE3) chemically competent E. coli cells. The transformed cells were plated on Luria agar plates containing kanamycin (25 μg/mL) and incubated overnight at 37° C. to obtain single colonies. The starter liquid cultures inoculated with colonies were grown in Luria broth (LB) with kanamycin (25 μg/mL) and inoculated into large cultures at a starting optical density (OD600) of ˜0.01. The expression cultures were incubated at 37° C. at 230 rpm until OD600 approached ˜0.7. The cultures were then induced with 0.4 mM IPTG. The expressed aminopeptidase was purified as described above for recognizers. For conditioning, the aminopeptidase protein was dialyzed against 50 mM MOPS pH 8.0/60 mM potassium acetate and then exposed to cobalt acetate at a final concentration of 400 μM for 1-1.5 h at 65° C. to form the active dodecamer complex. The conditioned aminopeptidase preparation was dialyzed further against 50 mM MOPS pH 8.0/60 mM potassium acetate, aliquoted, and flash frozen.

### Peptide Loading, Recognition, and Dynamic Sequencing

The semiconductor chip was placed in the sequencing device and a chip check was performed to test electronic circuit function and to optimize laser coupling alignment. The chip was then removed from the device socket and the chip was washed twice with 50 μL of 70% isopropanol, followed by four washes with 30 μL of wash buffer (50 mM MOPS pH 8.0, 60 mM potassium acetate, 50 mM glucose, 20 mM magnesium acetate, and surfactant mix) through a flow cell attached to the chip. A second chip check was then performed. The laser was then blocked via an integrated software-controlled shutter, peptide complex was added to a final concentration of 1-10 nM and mixed thoroughly, and the chip was incubated for 15 min. The chip was then washed six times with wash buffer, followed by addition of an imaging solution (wash buffer with 5 mM Trolox and an oxygen scavenging system). The laser was unblocked and the occupancy percentage (target 10-30%, Poisson distributed) was recorded by acquiring a photobleaching signal from a fluorophore attached to the peptide complex during 5 min of laser illumination. For NAA recognition-only assays, after peptide loading, labeled recognizer was added to a final concentration of 50 nM PS610, 100 nM PS691, or 250 nM PS961 (as indicated according to the experiment), and data was recorded for 10 hours. For dynamic sequencing assays, after peptide loading, a mixture of labeled recognizers was added to obtain final concentrations of 50 nM PS610, 100 nM PS691, and 250 nM PS961. Data was recorded for 15 min. The laser was then blocked briefly and aminopeptidases were added to the sequencing reaction via the flow cell and mixed thoroughly (final concentration 2-8 μM PhTET2 and/or 20-80 μM PhTET3, as indicated according to the experiment). The laser was then unblocked, and data was recorded for 10 hours. For all runs, 30 μL of mineral oil was added to fluid reservoirs at each port of the flow cell to prevent evaporation during the run.

### Signal Processing and Trace Segmentation

The measured signal on-chip comprises various noise components, the most dominant one being due to fluorescent emissions from diffusing recognizers in the reaction chamber. The pulse caller algorithm for a given reaction chamber starts by estimating the statistical properties of this background noise component. Once an estimate within certain error bounds has been established, the algorithm works in an online fashion observing new frames of data as they are generated. At each point in time, the algorithm maintains state indicating whether the signal is due to the background component only or a pulse from a recognizer-NAA interaction is being observed. The state transition from background to pulse is triggered using an edge detection test where the shift in signal is expected to be significant with respect to the background component's statistical distribution. The state transition from pulse to background is triggered when a small window of the most recent frames of the signal appears to conform to the background component's distribution again. The algorithm maintains an updated model of the background component as new background frames are observed. This provides robustness against drift in the signal intensity together with a feedback control loop that maintains a stable optical coupling of the laser into the chip based on any such detected drift. As detected pulses can be due to true recognizer-to-dipeptide interaction events as well as other occasional transient noise spikes, a downstream filter layer is employed to test the significance of pulse events based on their duration, intensity, and noise patterns within the context of the full timeline of the run and the entire dataset of reaction chambers.

Initial regions are determined by performing a sliding window calculation of pulse rate along the time dimension of a series of pulses. Regions with a mean pulse rate >1 pulse/min are then subdivided according to a greedy bisection approach. Here, the pulses on the left and right of each potential split are assessed for statistically significant deviation in any of four separate pulse properties-intensity, time bin ratio, pulse duration, and interpulse duration-using a Mann-Whitney U Test. To define RSs, the split point with the lowest p-value for any of the four properties is used to sub-divide the region and the process continues until no regions remain with a candidate split point with p-value <10⁻⁵ in any comparison. In this manner, transitions from one RS to the next in a region of continuous pulsing are determined a priori on the basis of changes in fluorescence properties of pulsing kinetics. The resulting regions are called recognition segments (RSs).

### Recognition Segment Classification

RS classification for reactions containing single synthetic peptides was performed using an unsupervised clustering algorithm. A subset of RSs including those with mean signal-to-noise ratio of their constituent pulses of >3 were used to pre-train a Gaussian mixture model (GMM) to identify approximate centroids for each of N classes of recognition, where N equals the number of expected recognizable peptide states with F, Y, W, L, I, V, or R at the N-terminus. Identified clusters were assigned to recognizable peptide states by matching the predominant order of cluster sequences observed to the expected amino acid sequence and by using prior knowledge of dye properties to identify the binders active during each RS. Subsequent rounds of GMM fitting were performed on all RSs matching the expected order of these events to refine the GMM model until no further sequences appeared in the expected order. The final model was then applied to all RSs in a given reaction.

RS classification for reactions containing library prepared peptides and mixes of peptides was performed using a random forest classifier that was pre-trained on annotated RS pulse features from prior synthetic peptide experiments. Unless otherwise noted, figures and statistics produced from classified RSs are derived from reaction chambers containing the expected sequence of RSs.

### Molecular Dynamics and Binding Energy Calculation

Homology models of PS961 complexed to peptide were generated using an internal crystal structure, mutations were applied and optimized using protCAD prior to molecular dynamics. AMBER20 implicit solvent molecular dynamics simulations using the generalized Born solvation potential were performed using the ff19SB force field with no atomic distance cutoff. Minimization was performed using steepest descent, followed by conjugate gradient minimization. The system was thermalized from 0 to 300K using Langevin dynamics and a collision frequency of 3 ps⁻¹. Molecular dynamics simulations of the equilibrated recognizer-peptide complex, free recognizer and free peptide were independently run for 5 nanoseconds at 300 K to perform the binding energy calculation using MMPBSA. Where 125 frames, each containing 10,000 2 femtosecond steps, were used for the calculation from the three simulations. Binding energy and the decomposition of all residues contributing to the binding energy was computed in 0.15 M salt concentration.

### Example 2. Peptide Identification Using Modeled Proteome-Wide Kinetic Signatures

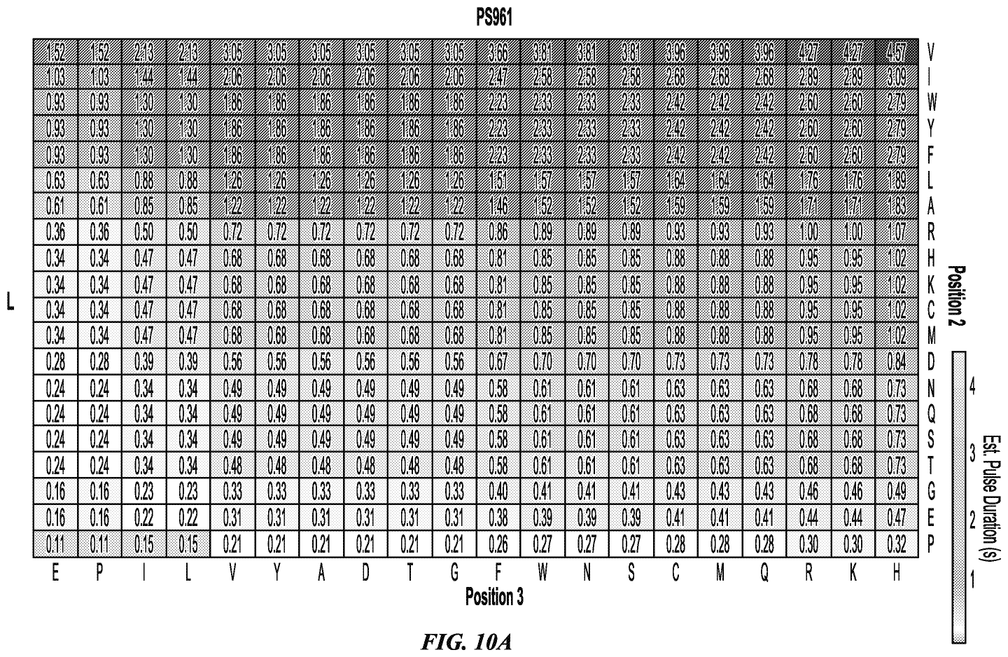

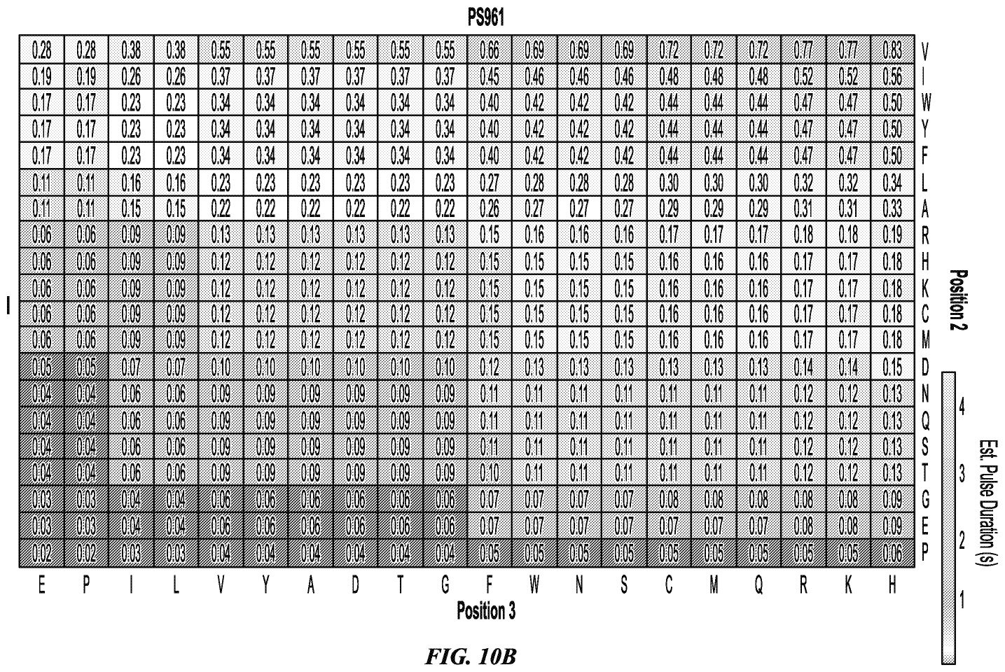

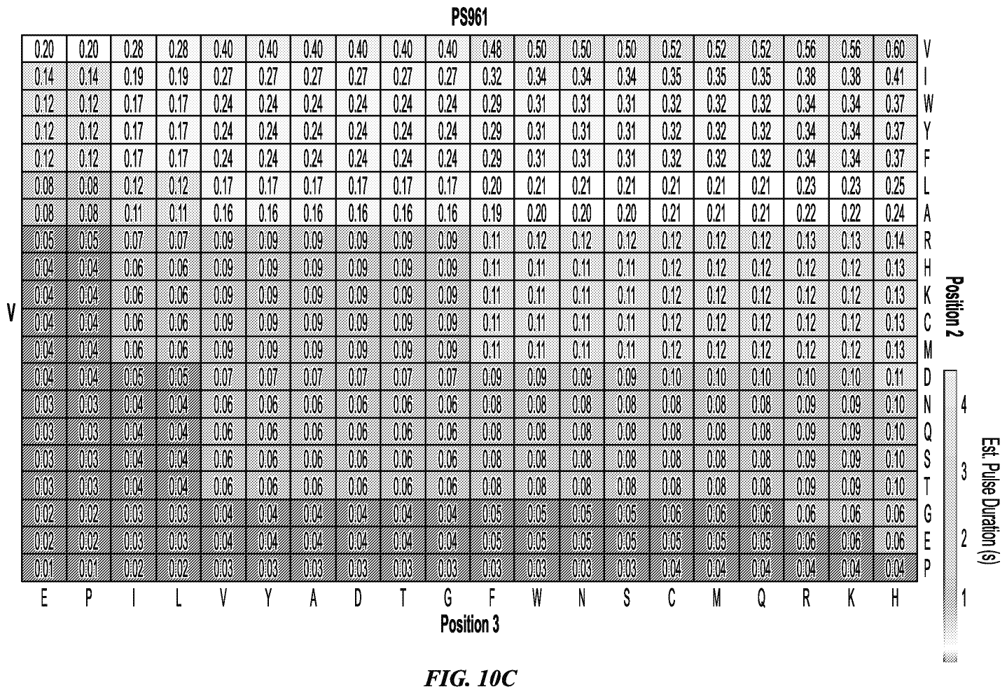

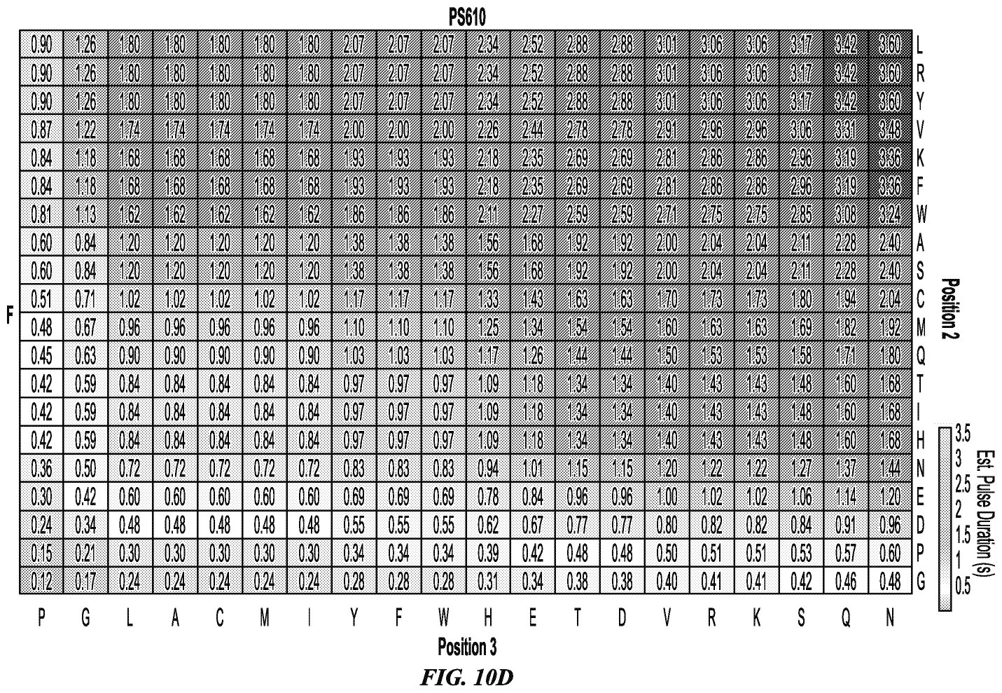

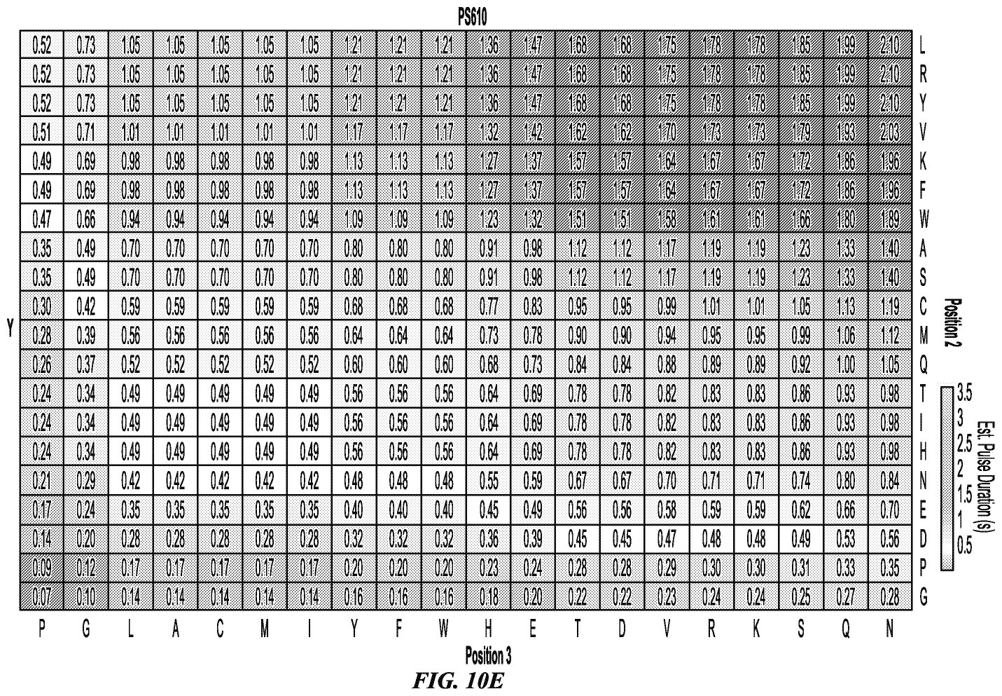

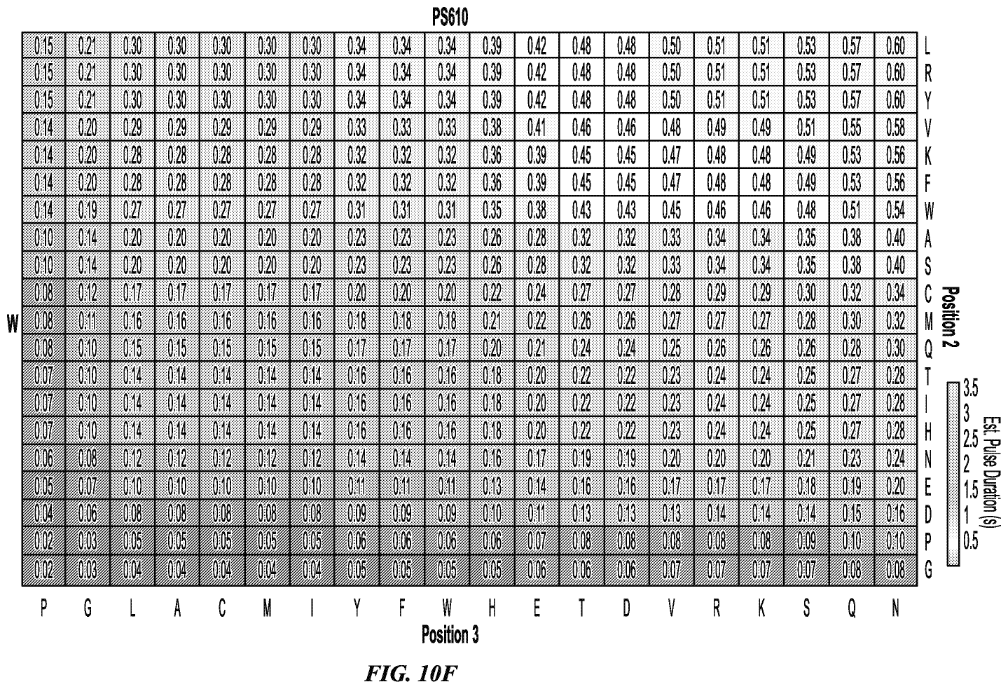

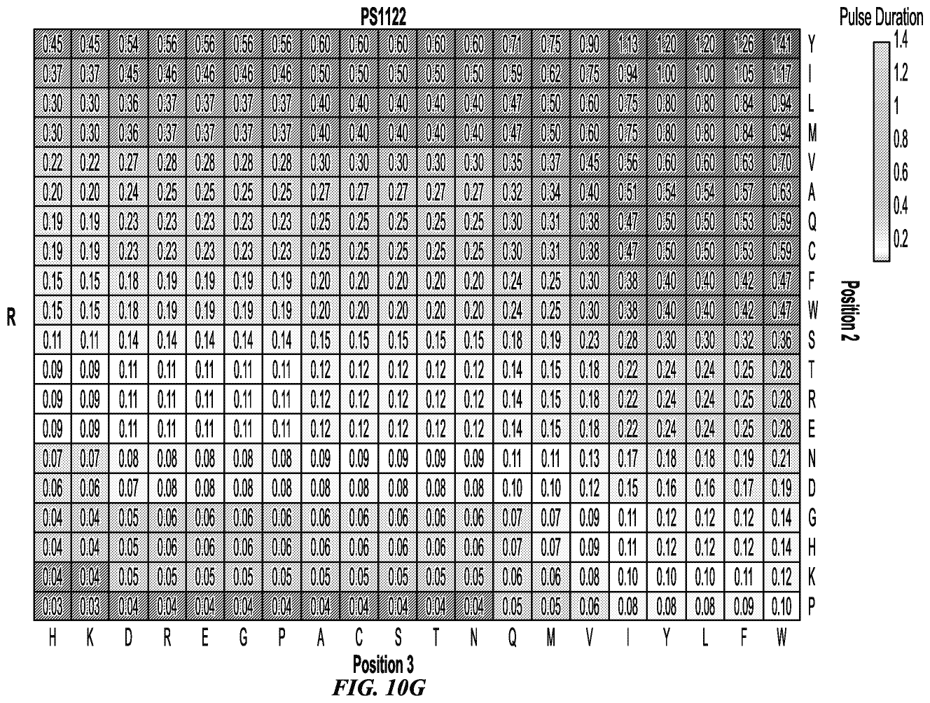

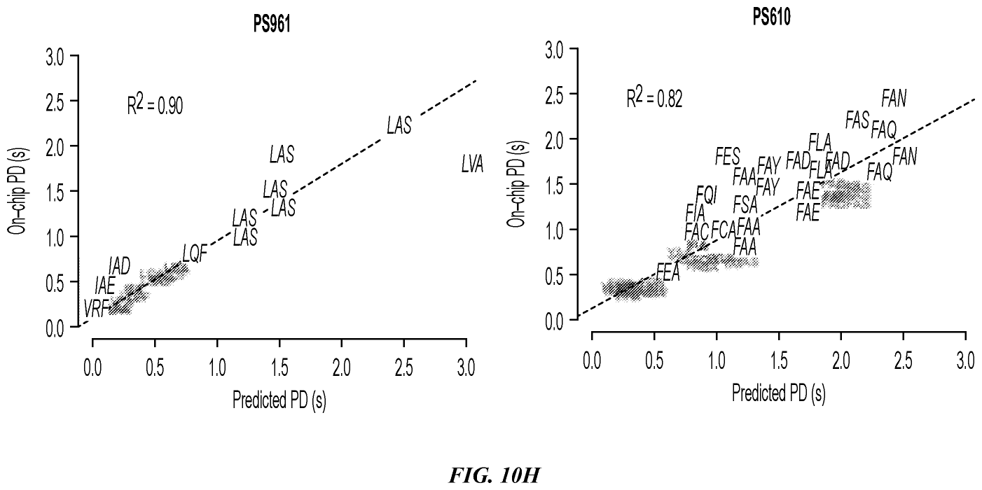

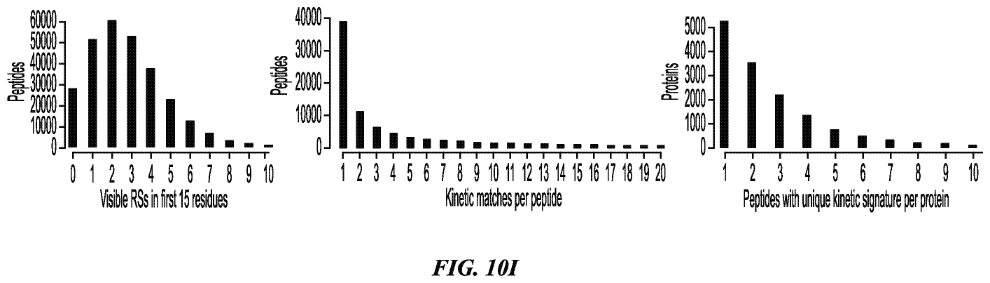

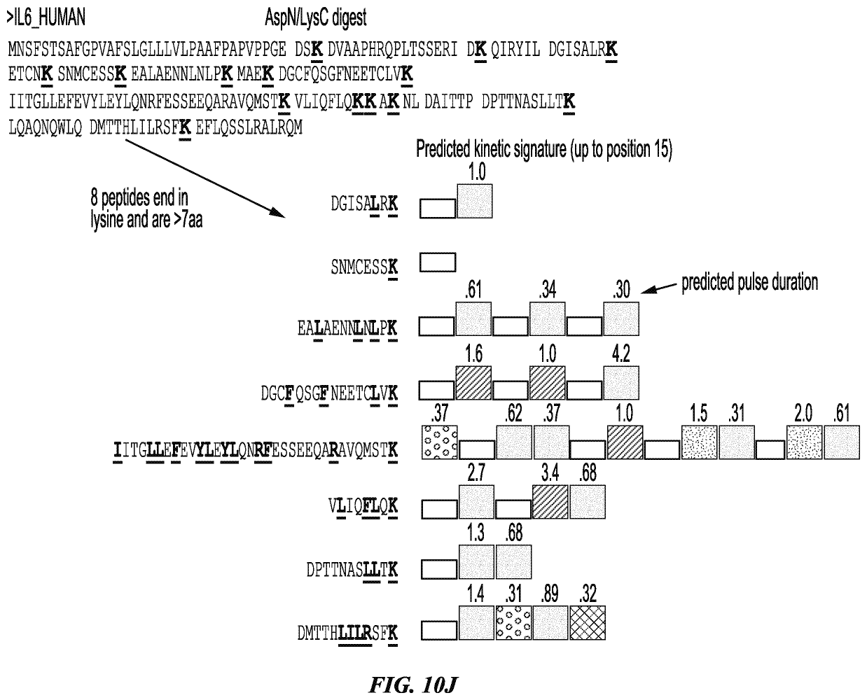

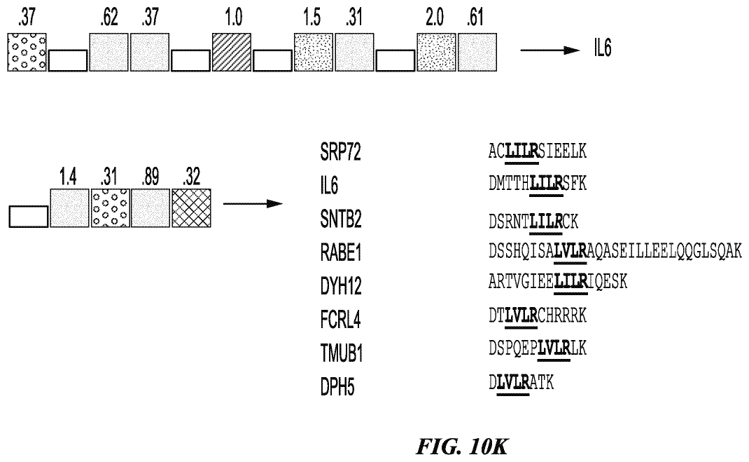

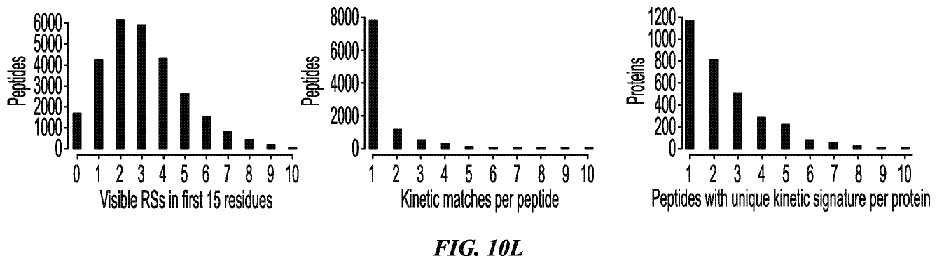

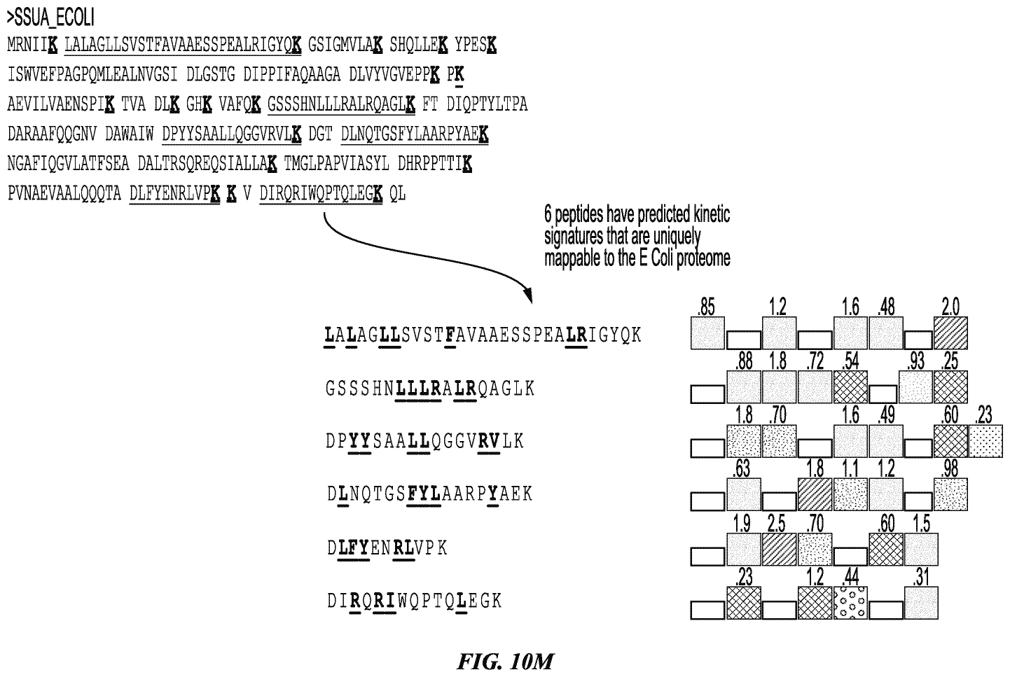

Sequencing and biochemical data was used to determine predicted pulse durations for recognizers binding all possible tripeptide targets. FIGS. 10A-10C show heatmaps of predicted pulse durations for PS961 binding tripeptide targets having leucine (FIG. 10A), isoleucine (FIG. 10B), or valine (FIG. 10C) at the N-terminal position. FIGS. 10D-10F show heatmaps of predicted pulse durations for PS610 binding tripeptide targets having phenylalanine (FIG. 10D), tyrosine (FIG. 10E), or tryptophan (FIG. 10F) at the N-terminal position. FIG. 10G shows a heatmap of predicted pulse durations for PS1122 binding tripeptide targets having arginine at the N-terminal position. The predicted pulse durations displayed high correlation with actual pulse durations from on-chip experimental results for PS961 (FIG. 10H, left plot) and PS610 (FIG. 10H, right plot).

With this database of predicted tripeptide pulse durations, the expected kinetic signature of every peptide in the human proteome can be modeled, which could provide an improved understanding and utilization of the ability to identify proteins from sequencing output. A kinetic signature is an average representation of the sequencing behavior of a peptide on-chip, as detailed above in Example 1. The information in kinetic signatures derived from single-molecule traces dramatically improves the ability to map sequencing data to the proteome (e.g., compared to methods based on alignment of text strings, as in DNA sequencing). Kinetic information can include, for example, pulse duration, interpulse duration, and recognition segment (RS) duration.

Kinetic information can improve mapping data to the proteome because recognizers contact (at least) the two adjacent downstream residues when they bind a peptide, not just the N-terminal residue. In this manner, they indirectly sense all 20 amino acids, and this information is encoded in the average pulse duration (and also potentially in IPD and RS duration). Additionally, adjacent visible residues in a peptide are represented on average by immediately adjacent RSs (i.e., there is only a consensus gap between two RSs if there is at least one invisible amino acid between them).

To prepare a model demonstrating the ability to uniquely map peptides to the human proteome (with the recognizers PS961, PS610, and PS1122), an in silico digest of the proteome with AspN/LysC was performed, followed by a selection of all peptides that end in lysine (used for on-chip immobilization) and are greater than 7 amino acids in length. The results are shown below.

| Human proteins (SWISS-Prot): | 20,595 proteins |
|---|---|
| Peptides from AspN/LysC digest: | 1,148,192 |
| Peptides ending in lysine: | 652,225 |
| Peptides with >7 amino acids: | 273,112 |

A predicted pulse duration was assigned to every visible amino acid in the set of 273,112 peptides (positions with predicted average PD of less than 0.18 s were treated as invisible). The distribution of predicted RSs in the first 15 residues is shown in FIG. 10I (left plot). 82,068 peptides contained 4 or more RSs (and thus were considered potentially informative). Kinetic signatures were created for each of these peptides.

The kinetic signature contains the expected binder and average PD at each visible position, and a gap to represent runs of one or more invisible amino acids. Next, for each peptide, the number of peptides with identical kinetic signatures was determined (signatures were considered identical if they had the same order of RSs and gaps, and the predicted PDs at each RS were somewhat similar (shorter PD not less than half the longer PD in any pairwise comparison)). According to this analysis, 38,849 out of 82,068 peptides produced a unique kinetic signature with no other matches in the human proteome. A further 10,571 peptides had only 1 other match. The distribution of kinetic matches per peptide is shown in FIG. 10I (middle plot). 14,167 proteins (69% of all proteins) contained at least one uniquely mappable peptide. On average, there were 2.5 uniquely mappable peptides per protein. The distribution of uniquely mappable peptides per protein is shown FIG. 10I (right plot).

To further illustrate this data and how it might be used to model protein behavior, results with IL6 protein are shown in FIG. 10J (for simplicity, residues immediately before C-terminal lysine were treated as invisible and XP motifs were treated as cleavable). As shown in FIG. 10J, two peptides contain at least 4 RSs. As shown in FIG. 10K, one of these peptides maps uniquely to IL6, and the other peptide matches the kinetic signature of 8 different peptides from 8 proteins.

To provide an illustrative example using a smaller proteome, the E. coli proteome (containing only 4,392 proteins) was analyzed as described above for the human proteome. The results are shown below.

| | |
|---|---|
| E. Coli Proteins: | 4,392 |
| Peptides from AspN/LysC digest: | 126,439 |
| Peptides ending in lysine: | 59,697 |
| Peptides with >7 amino acids: | 28,046 |
| Peptides containing 4+ visible | 9,925 |
| RSs in first 15 residues: | |
| Peptides having unique kinetic | 7,740 (78%) |
| signatures: | |
| Proteins having at least one peptide | 3,527 |
| with 4+ RSs in first 15 residues: | |
| Proteins having at least one uniquely | 3,187 out of 3527 |
| mappable peptide (mean 2.4 peptides): | |

The distribution of predicted RSs in the first 15 residues is shown in FIG. 10L (left plot). 9,925 peptides contained 4 or more RSs (and thus were considered potentially informative). Kinetic signatures were created for each of these peptides. For each peptide, the number of peptides with identical kinetic signatures was determined. According to this analysis, 7,740 out of 9,925 peptides produced a unique kinetic signature with no other matches in the E. coli proteome. The distribution of kinetic matches per peptide is shown in FIG. 10L (middle plot). 3,187 proteins contained at least one uniquely mappable peptide. On average, there were 2.4 uniquely mappable peptides per protein. The distribution of uniquely mappable peptides per protein is shown FIG. 10L (right plot). To illustrate this data and how it might be used to model protein behavior, results with a protein from E. coli containing 6 peptides that are uniquely mappable are shown in FIG. 10M.

These results demonstrate the utility of a kinetics-centric view of peptide identification. This view also provides the ability to accurately model the informatic impact of changes to reaction conditions, such as the addition of new recognizers, increases in recognizer pulse duration, changes in frame rate, and addition of new dye labels.

### Example 3. Selection of N-Terminal Alanine and Valine Binding Variants by Yeast Display

The gene encoding PS557 was used as the template for an error-prone PCR in which multiple nucleotide changes were introduced to create mutated PS557 proteins. The mutated protein library was transformed into yeast and used for yeast display and flow cytometry, where selections were performed against the peptide of interest. In brief, the protein can be labeled with a tag, such as a myc tag, and a fluorescently labeled antibody towards this tag identifies cells expressing the protein. The peptide of interest is biotinylated and can be labeled with a streptavidinylated fluorophore to identify yeast cells displaying proteins that are bound to the peptide. The mixture of yeast cells, peptide, and fluorophores is incubated for 1 hour at room temperature, and then two-color FACS performed and the double-positive cells obtained.

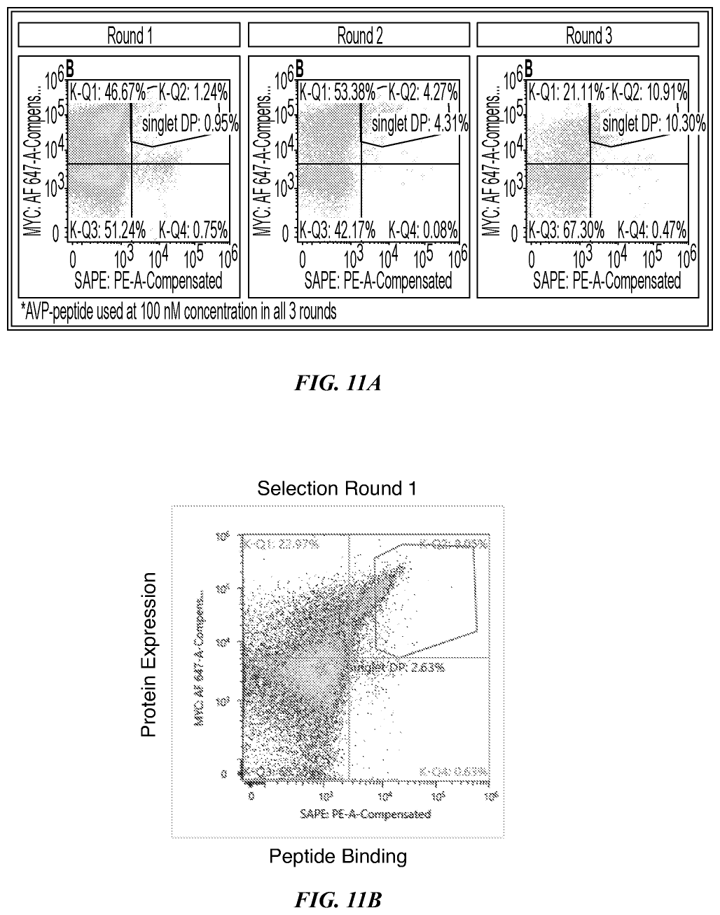

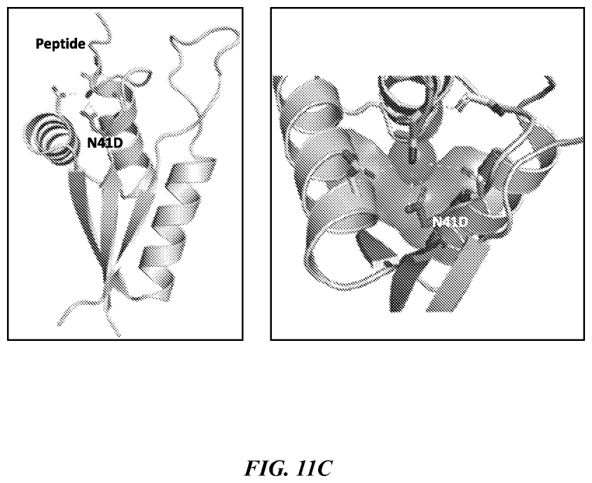

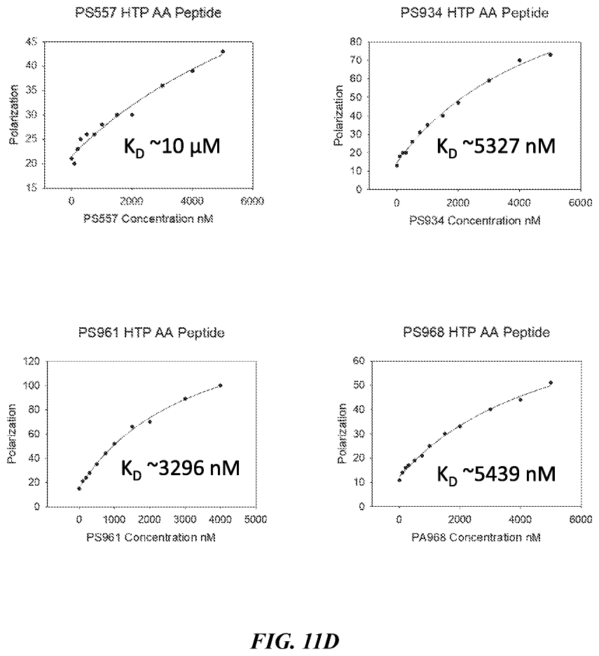

In this example, selections were performed using peptides with amino acid residues V or A at the N-terminus. After 3 rounds of selection against each peptide, the samples were sent for next-generation sequencing and the results used to rationally design additional libraries for another iteration of directed evolution or individual proteins tested in biochemical assays. FIG. 11A shows results from the FACS selections for 3 rounds of selection, with flow cytometry plots shown as cells expressing protein (y-axis) against cells binding to AVP-peptide (x-axis). FIG. 11B shows results from one round of selection of an error prone PCR library mixture, with a flow cytometry plot shown as cells expressing protein (y-axis) against cells binding to 1 μM AV or AI peptide (x-axis). In each of the flow cytometry plots shown in FIGS. 11A-11B, signals in the top right quadrant are indicative of more binding. A selection of hits obtained from sequencing of the alanine and valine libraries are given in Table 3.

TABLE 3
Hits obtained from sequencing of alanine and valine libraries.

| | | Percent of | | | Percent of |
|---|---|---|---|---|---|
| | | Sequenced | | | Sequenced |
| Sample | Mutations | pool | Sample | Mutations | pool |
| Valine sort 1 | WT | 40 | Alanine sort 1 | WT | 61 |
| | N41D | 9 | | D93N | 3 |
| | S6N_V52E_R106C | 7 | | D14G | 2 |
| | R106C | 6 | | E63K | 2 |
| | R106H | 6 | | V51I | 2 |
| | T27I_K70E | 4 | | E63G | 2 |
| | K70E | 3 | | V38I | 2 |
| | A7V | 2 | | E63V | 2 |
| | G25D_K30E_R106H | 2 | | | |
| | Q32L_K70E_A105V | 2 | Alanine sort 2 | WT | 44 |
| | Q55H | 2 | | I12F_N41D_Q55R | 14 |
| | A117E | 2 | | N41D | 13 |
| | R102H_R106C | 2 | | V72M | 7 |
| | I12V_P16Q | 2 | | A7G_N41D_Q55R | 5 |
| Valine sort 2 | A4T | 2 | | E63K | 3 |
| | T27I | 2 | | F58L_V72M | 3 |
| Valine sort 3 | WT | 69 | | A17V | 3 |
| | N41D | 5 | | E22V_N41D | 2 |
| | P18S_Q32R | 3 | | P61S_E63K | 2 |
| | K108R | 2 | Alanine sort 3 | E63K | 21 |
| | T3I_D14N | 2 | | N41D | 16 |
| | A4T | 2 | | A7G_N41D_Q55R | 14 |
| | I12V | 2 | | I12F_N41D_Q55R | 8 |
| | A7T | 2 | | P62R_Q75L | 6 |
| | Y66F | 2 | | E22V_N41D | 5 |
| Valine sort 4 | WT | 58 | | V72M | 4 |
| | Q55H | 4 | | P62R | 4 |
| | H87R | 3 | | P61S_E63K | 3 |
| | K108R | 3 | | M1T_E63K | 3 |
| | D14N_N41D | 2 | | R31H_Q55R | 3 |
| | N41D | 2 | | L68M | 3 |
| | I12V | 2 | | F58L_V72M | 2 |
| | M1I | 2 | | E63G | 2 |
| | A7T | 2 | | E63G_R106H | 2 |
| | V52A | 2 | | | |
| | Y66F | 2 | | | |
| | Y100C | 2 | | | |
| Valine sort 5 | R106C | 20 | | | |
| | R106H | 20 | | | |
| | S6N_V52E_R106C | 11 | | | |
| | N41D | 9 | | | |
| | T27I_K70E | 6 | | | |
| | G25D_K30E_R106H | 5 | | | |
| | K70E | 5 | | | |
| | WT | 5 | | | |
| | R102H_R106C | 3 | | | |
| | T27I_N41D_K108R | 3 | | | |
| | R106S | 3 | | | |
| | R106G | 2 | | | |
| | D43E_I79T_R106G | 2 | | | |

The mutation N41D was selected as a top hit, which can be rationalized via computational modeling. When residue N41 is mutated to D in silico, the Rosetta algorithm gives a more favorable binding energy with the valine peptide (−1.8) as compared to the wild-type PS557. This predicted binding energy difference is approximately that of 1-2 hydrogen bonds, and therefore would potentially result in a 5-10 fold difference in K_{D} experimentally. FIG. 11C illustrates the presumed binding of an alanine peptide to the mutated protein. The left panel of FIG. 11C shows a model of the entire PS557 protein with the peptide shown at the binding site forming hydrogen bond interactions with the N41D mutation. The right panel of FIG. 11C shows the hydrogen bond network overlayed with the negative (lighter shading) and positively charged (relevant) surface area.

The mutation V72M also appeared to be enriched by the selections, and can also be rationalized via computational modeling. Therefore, these two mutations were chosen to be combined together and the double-mutant expressed in E. coli and purified to be tested for binding activity. Similarly, other mutations were chosen from the NGS dataset and combined using rational design, to result in a panel of potential hits to be tested in a high throughput assay on the Octet platform.

In the high-throughput assay, Octet sensors are coated with the peptide of interest and dipped in buffer containing the purified protein. An approximation of relative binding can be obtained by comparing the response after 200 seconds for each protein. Each protein has approximately the same molecular mass and is used at the same concentration, so ranking the proteins by response level in this assay gives an approximation of which proteins have improved binding. Studying the on and off-rate can also give insight into the mechanism of binding. The response values for different peptides with the constructs selected from this round of selection are given in Tables 4 and 5, which show binding affinity of selected candidates as compared to the wild-type protein (PS557) in the high-throughput Octet assay. Responses (in nm) are given for each peptide with the two N-terminal residues of the peptide listed (Table 4: AA, VA, LA; Table 5: MA, IA, FA, WA, YA) after 200 see incubation during the association phase of incubation with a mutant protein candidate.

TABLE 4
Binding affinity of selected candidates
for AA, VA, and LA peptides.

| Protein | Mutations | AA | VA | LA |
|---|---|---|---|---|
| PS557 | WT | 0.01 | 5.9 | 9.8 |
| PS796 | N41D, V72M | 3.5 | 11.3 | 11.9 |
| PS797 | N41D, V72M, Q75L | 3.2 | 11.2 | 12 |
| PS798 | V72M, Q75L | 3.8 | 12.9 | 13.9 |
| PS824 | V72M, I12F_N41D_Q55R | 3.9 | 13.3 | 13.8 |
| PS829 | V72M, N41D, P62R_Q75L | 2.5 | 9.5 | 8.5 |
| PS829 | V72M, N41D, P62R_Q75L | 4.5 | 11.3 | 10.9 |
| PS831 | E63K, V72M, P62R_Q75L | 1.4 | 11 | 12.2 |
| PS833 | V72M, E22V_N41D | 1 | 8.6 | 10.7 |
| PS838 | V72M, N41D, P62R | 3 | 11.6 | 13.4 |
| PS852 | V72M, N41D, R31H_Q55R | 1.7 | 8.3 | 9.7 |
| PS801/ | E63K | 0.0017 | 5.7333 | 10.7026 |
| PS814 | | | | |
| PS808 | Q55R | 0.0027 | 5.7079 | 10.4143 |
| PS809 | V72M | 0.0226 | 7.7722 | 10.797 |
| PS819 | V72M, N41D, E63K | 0.5194 | 7.4185 | 11.0591 |
| PS828 | N41D, P62R_Q75L | 0.4688* | 3.2418* | 7.7465* |
| PS830 | E63K, V72M, N41D, | 0.2153* | 6.6407* | 8.5351* |
| | P62R_Q75L | | | |
| PS833 | V72M, E22V_N41D | 0.8494 | 10.4144 | 12.381 |
| PS847 | V72M, N41D_Q55R | 0.2885 | 6.6782 | 9.9157 |

TABLE 5
Binding affinity of selected candidates for MA, IA, FA, WA, and YA peptides.

| Protein | Mutations | MA | IA | FA | WA | YA |
|---|---|---|---|---|---|---|
| PS557 | WT | 1.6754 | 8.7912 | 0.0096 | 0.0049 | 0.0431 |
| PS796 | N41D, V72M | 8.4229 | 11.6038 | 1.3679 | 0.7606 | 1.2311 |
| PS797 | N41D, V72M, | 8.7055* | 11.5559* | 0.2024* | | |
| | Q75L | | | | | |
| PS798 | V72M, Q75L | 9.3717* | 12.6651* | 1.4066* | | |
| PS824 | V72M, | 9.5005* | 13.4909* | 0.6735* | 0.1651* | 1.3124* |
| | I12F_N41D_Q55R | | | | | |
| PS829 | V72M, N41D, | 4.7003* | 7.5118* | 0.7972* | 0.625* | 0.7488* |
| | P62R_Q75L | | | | | |
| PS829 | V72M, N41D, | 7.8569* | 8.8511* | 1.181* | | |
| | P62R_Q75L | | | | | |
| PS831 | E63K, V72M, | 4.8567* | 10.5071* | 0.7549* | | |
| | P62R_Q75L | | | | | |
| PS833 | V72M, | 5.112* | 9.2566* | 0.0509* | 0.005608* | 0.1168* |
| | E22V_N41D | | | | | |
| PS838 | V72M, N41D, | 8.0868 | 11.1833 | 0.3766 | 0.0581 | 0.5903 |
| | P62R | | | | | |
| PS852 | V72M, N41D, | 5.4778* | 9.3775* | 0.102* | 0.0292* | 0.1775* |
| | R31H_Q55R | | | | | |
| PS801/PS814 | E63K | 1.7522 | 9.0209 | 6.62E−03 | 0.0148 | 0.054 |
| PS808 | Q55R | 1.8761 | 9.2695 | 2.33E−03 | 2.43E−03 | 0.0539 |
| PS809 | V72M | 2.2305 | 8.5194 | 0.0032 | 1.17E−02 | 0.0027 |
| PS819 | V72M, N41D, | 3.7494 | 9.0637 | | | |
| | E63K | | | | | |
| PS828 | N41D, | 1.703* | 5.3196* | 0.5834* | | |
| | P62R_Q75L | | | | | |
| PS830 | E63K, V72M, | 2.1593* | 6.5138* | 0.0339* | | |
| | N41D, | | | | | |
| | P62R_Q75L | | | | | |
| PS833 | V72M, | 7.0145 | 10.7881 | 0.0889 | 0.0006 | 0.1093 |
| | E22V_N41D | | | | | |
| PS847 | V72M, | 3.2321 | 7.7665 | 0.059 | 0.0098 | 0.0607 |
| | N41D_Q55R | | | | | |

The results show that binding has been improved for both valine and alanine binding in some candidates, such as PS824, which was selected for further characterization. Upon more quantitative measurement, by fluorescence polarization, binding has been improved by 5-fold for the amino acid valine. The wild-type protein has a measured K_{D} of 1174 nM, whereas the PS824 mutant containing mutations I12F, N41D, Q55R, and V72M has a K_{D} of 205 nM for a peptide with an N-terminal valine (Table 6). Similarly, clone PS852 containing R31H, N41D, Q55R, and V72M has a K_{D} of 142 nM for a valine peptide, as measured by fluorescence polarization. FIG. 11D shows example results from fluorescence polarization studies comparing kinetics of N-terminal alanine peptide binding for selected PS557 variants. The results shown in FIG. 11D were obtained using mixtures containing PBS buffer with 0.01% Tween 20, peptide (AAKLDEESILKQ{LYS(FITC)}(SEQ ID NO: 1075)) at a concentration of 100 nM, and protein at concentrations spanning 100-5000 nM.

TABLE 6
Binding affinity of selected candidates as measured by fluorescence polarization.

| | LA | IA | | | | |
|---|---|---|---|---|---|---|
| | Peptide | Peptide | VA | AA | MA | |
| | (Kd, | (Kd, | Peptide | Peptide | Peptide | |
| Protein | nM) | nM) | (Kd, nM) | (Kd, nM) | (Kd, nM) | Substitutions |
| PS557 | 52 | 289 | 680 | 10000 | — | |
| PS769 | — | — | 276 | 8700 | — | N41D |
| PS816 | — | — | 266 | 7593 | 805 | N41D, V72M |
| PS824 | 10 | — | 205 | 6771 | 648 | N41D, V72M, Q55R, I12F |
| PS833 | — | — | 312 | 7080 | 1036 | N41D, V72M, E22V |
| PS852 | 19 | — | 142 | 5142 | 1140 | N41D, V72M, Q55R, R31H |
| PS857 | 7 | — | 487 | 14995 | 746 | N41D, V72M, L68M |
| PS934 | — | — | — | 5327 | 325 | N41D, V72M, L68M, P62R |
| PS961 | — | — | — | 3296 | 143 | N41D, V72M, Q55R, E63S, |
| | | | | | | L68M, Y100R |
| PS968 | — | — | — | 5439 | 395 | V72M, Q55R, Y100R |

Concurrently, the NGS dataset was also used to design second-generation libraries where additional mutations were layered on top of the clones selected from the first round of directed evolution. Both error-prone PCR and targeted mutagenic libraries were created and more rounds of selection performed as described, for a second iteration of directed evolution. The top hits are summarized in Tables 7-8 and illustrate a snapshot of the best clones obtained from these methods.

TABLE 7
Candidate amino acids in PS557 for mutation.

| | |
|---|---|
| Top Candidates | Additional Candidates |

| PS557 | Amino Acid | PS557 | Amino Acid |
|---|---|---|---|
| Amino Acid | Substitutions | Amino Acid | Substitutions |
| E22 | V | A7 | G |
| R31 | H | I12 | F |
| N41 | D | P21 | S |
| Q55 | R, H | V23 | M |
| P62 | R | R26 | C |
| E63 | A, G, S, K | T27 | I, S |
| L68 | M | K30 | R |
| V72 | M | Q32 | H |
| Q75 | L | P33 | T |
| Y100 | R | Y35 | C |
| L39 | M | H36 | Y |
| D42 | L, P | D42 | Y |
| H45 | C, F | D43 | N |
| V50 | F, Y, A | D44 | Y |
| M111 | A, S | V51 | L |
| | | F58 | L |
| | | H60 | I, Y |
| | | G65 | C |
| | | I79 | V |
| | | D93 | G |
| | | F98 | V |
| | | Y100 | S |
| | | R106 | G, H |

TABLE 8
Candidate amino acid combinations in PS557 for mutation.

| PS557 Positions | Mutations | Protein |
|---|---|---|
| 41, 72 | N41D, V72M | PS816 |
| 31, 41, 55, 72 | R31H, N41D, Q55R, V72M | PS852 |
| 22, 31, 41, 68, 72, 75 | E22V, R31H, N41D, L68M, V72M, Q75L | PS927 |
| 41, 55, 68, 72 | N41D, Q55R, L68M, V72M | PS933 |
| 41, 62, 68, 72 | N41D, P62R, L68M, V72M | PS934 |
| 31, 41, 68, 72 | R31H, N41D, Q55R, V72M | PS938 |
| 45, 111 | H45F, M111A | |
| 42, 45, 72 | D42P, H45C, V72M | |
| 42, 45, 50, 111 | D42L, H45C, V50F, M111A | |
| 50, 111 | V50Y, M111A | |
| | V50F, M111A | |
| | V50F, M111S | |
| 39, 72 | L39M, V72M | |
| 41, 50, 72, 111 | N41D, V50A, V72R, M111A | |
| 12, 41, 55, 72 | I12F, N41D, Q55R, V72M | PS824 |
| 7, 41, 55, 72 | A7G, N41D, Q55R, V72M | PS821 |
| 22, 31, 41, 62, 72, 75 | E22V, R31H, N41D, P62R, V72M, Q75L | PS944 |
| 41, 63, 72, 75 | N41D, E63K, V72M, Q75L | PS896 |
| 41, 63, 72, 106 | N41D, E63K, V72M, R106H | PS907 |
| | N41D, V72M, Q75L, E22V, R31H | PS945 |
| | N41D, V72M, E22V, R31H | PS946 |
| | N41D, V72M, I12F | PS947 |
| | N41D, V72M, R31H | PS948 |
| | N41D, V72M, L68M, I12F, Q55R, P62R, Q75L, E22V, R31H, | PS949 |
| | D43N, D44Y, R106H | |
| | N41D, V72M, L68M, D43N, D44Y, R106H | PS950 |
| | N41D, V72M, L68M, D44Y, R106H | PS951 |
| | N41D, V72M, L68M, R106H | PS952 |
| | N41D, V72M, L68M, D44Y | PS953 |
| | N41D, V72M, L68M, D43N | PS954 |
| | N41D, V72M, R31H, Q55R, I12F, P62R, Q75L, E22V | PS955 |
| | N41D, V72M, R31H, Q55R, Q75L | PS956 |
| | N41D, V72M, R31H, Q55R, P62R | PS957 |
| | N41D, V72M, R31H, Q55R, E22V | PS958 |
| | N41D, Q55R, H60I, V72M | PS960 |
| | N41D, Q55R, E63S, L68M, V72M, Y100R | PS961 |
| | N41D, Q55R, P62Y, E63A, V72M, Y100R | PS962 |
| | N41D, Q55R, E63A, V72M, Y100R | PS963 |
| | N41D, Q55R, E63W, V72M, R106H | PS964 |
| | N41D, E63S, L68M, V72M, R106H | PS965 |
| | N41D, E63A, V72M, R106H | PS966 |
| | N41D, Q55R, E63A, V72M, Y100R | PS967 |
| | Q55R, V72M, Y100R | PS968 |
| | N41D, Q55R, E63S, L68M, V72M, Y100R, R106H | PS969 |
| | Q55R, E63S, V72M | PS970 |
| | R31H, N41D, Q55R, V72M, R106H | PS971 |
| | R31H, N41D, Q55H, V72M | PS972 |
| | R31H, N41D, V50L, Q55R, V72M | PS973 |
| | R31H, N41D, V50I, Q55R, V72M | PS974 |
| | R31H, N41D, Q55R, V72F | PS975 |
| | R31H, N41D, D43N, D44Y, Q55R, V72M | PS976 |
| | N41D, Q55H, V72M | PS978 |
| | A7G, N41D, V52E, Q55R, V72M | PS979 |
| | I12F, N41D, Q55R, P62T, V72M | PS980 |
| | N41D, Q55R, E63D, V72M | PS981 |
| | N41D, F58L, V72M | PS982 |
| | V23M, N41D, Q55R, V72M, D93G | PS983 |
| | H36Y, N41D, Q55R, V72M | PS984 |
| | P33T, N41D, V72M | PS985 |
| | K28V, N41D, R67H, V72M | PS986 |
| | N41D, V72M, I79V | PS987 |
| | K28V, N41D, L68M, V72M | PS988 |
| | K30R, N41D, Q55R, V72M | PS989 |
| | K28V, N41D, V72M | PS990 |
| | I12F, K30I, N41D, Q55R, V72M | PS991 |
| | Y35C, N41D, Q55R, V72M | PS992 |
| | P21S, N41D, V72M | PS993 |
| | K30E, N41D, V72M | PS994 |
| | R26C, N41D, V72M | PS995 |
| | R26S, N41D, V72M | PS996 |
| | V23M, N41D, Q55R, V72M, F98V | PS997 |
| | Q32H, N41D, P61S, E63K, R67C, V72M | PS998 |
| | I12F, N41D, Q55R, H60Y, V72M | PS999 |

### Example 4. Improved N-Terminal Amino Acid-Binding Proteins Using SNAP Display

The gene encoding PS557 was cloned into a vector to be used for SNAP display such that the SNAP tag is fused to the N-terminus. During SNAP-display, the SNAP protein tag reacts with and binds covalently to the benzylguanine (BG) molecule, which is added to the ends of the DNA template coding for itself. This allows the connection to be made between phenotype and genotype of the protein provided that the protein is expressed in a droplet emulsion using an in vitro transcription translation system. The protein-DNA complex is exposed to the desired peptide which is bound to a magnetic bead, and the complexes that are not bound are washed away and the high affinity binders are eluted off of the beads.

In these studies, a library of mutant PS557 genes was created using error prone PCR and targeted mutagenesis, rationally designed based on computational modeling and prior results. The library was selected for binding to alanine, valine, and methionine peptides using SNAP display. The naïve and selected libraries were each sequenced, and the NGS data used to determine enrichment of clones from the different rounds of selection.

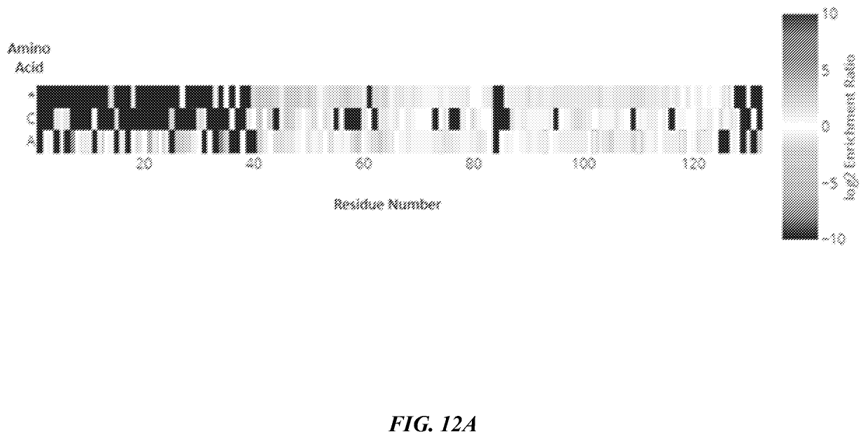

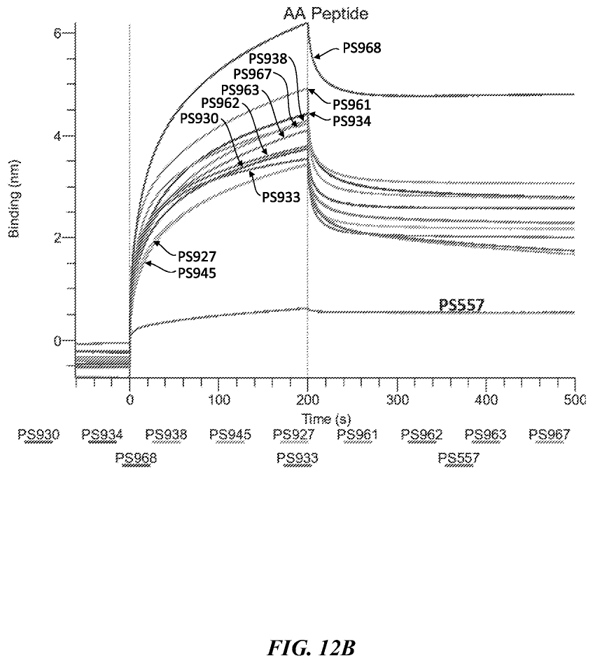

Comparing the frequency with which a given protein sequence appears in the library before and after selection, clones can be ranked by potential affinity for the peptide. Most sequences display low affinity and are selected out. The results showed that defective clones (e.g. clones with stop codons) are selected out, giving confidence in the method. FIG. 12A is a heatmap showing enrichment of mutations in the PS557 protein. The amino acid that the residue was changed to is listed on the left, and the residue number across the bottom. Each rectangle represents enrichment of that mutation in the selected library compared to the naïve library (dark is not enriched, light is enriched). Rectangles represent the wild-type residue at this position. A subset of mutations is shown for simplicity, a stop codon, cysteine, or alanine residue.

As shown in FIG. 12A, the stop codons are mostly not enriched at all positions in the sequence, listed by residue number. However, for example, an alanine mutation is enriched in many positions, illustrated by the light rectangles, and is more enriched than for example, cysteine.

Four rounds of selection were performed on a library of mutant PS557 proteins. The most enriched sequences from this round of directed evolution against an alanine peptide are given in Table 9, which shows mutations in the PS557 sequence that were found to be enriched after four rounds of SNAP-selection against an N-terminal alanine peptide. The enrichment was calculated by the percentage abundance of the clone in the NGS data from the 4th round sequencing divided by the abundance in the naïve library.

TABLE 9
Enriched mutations in the PS557 sequence.

| Mutations | Enrichment |
|---|---|
| N41D, Q55R, H60I, V72M | 319.81 |
| N41D, Q55R, E63S, L68M, V72M, | 165.55 |
| Y100R | |
| N41D, Q55R, P62Y, E63T, V72M, | 158.03 |
| Y100R | |
| N41D, Q55R, E63A, V72M, Y100R | 154.26 |
| N41D, Q55R, E63W, V72M, R106H | 132.96 |
| N41D, E63S, L68M, V72M, R106H | 124.16 |

As shown by the results in Table 9, many of the enriched sequences contained similar mutations. The library used for this iteration of directed evolution was designed based on hits from three previous rounds of directed evolution and selections which can be explained by tracing the evolution of one particular clone, for example: N41D, Q55R, E63S, L68M, V72M, Y100R. The mutation N41D was first identified as enriched after selection of an error-prone PCR library in yeast display (Table 10, directed evolution round 1).

TABLE 10
PS557 mutations identified in hits enriched from selections with an alanine-bearing peptide

| Enriched Clones: Round 1 | | Enriched Clones: Round 2 | | Enriched Clones: Round 3 | |
|---|---|---|---|---|---|
| Rank | Genotype | Rank | Genotype | Rank | Genotype |
| 1 | I12F_N41D_Q55R | 1 | N41D_Q55R | 1 | L68H_E71T |
| 2 | N41D | 2 | I12F_N41D_Q55R | 2 | D44R |
| 3 | V72M | 3 | A7G_N41D_Q55R | 3 | Y49T |
| 4 | A7G_N41D_Q55R | 4 | V72M | 4 | E63Q |
| 5 | E63K | 5 | A7G_N41D_Q55R_E129K | 5 | Y49D_K70L_V72A_D73H |
| 6 | F58L_V72M | 6 | A4E_I12F_N41D_Q55R_Q124H | 6 | W40E_V72Y |
| 7 | A17V | 7 | A11V_R20C_T84N_E89G | 7 | Y47S_E63Q_A69L |
| 8 | E22V_N41D | 8 | N41D_H96R | 8 | Q48R_Y66Q_R67E |
| 9 | P61S_E63K | 9 | N41D_Q55R_V72M | 9 | W40H_Y47S_E63Q |
| 10 | E63G | 10 | N41D | 10 | P62Q_E63T |
| 11 | P62R_Q75L | 11 | N41D_Q55R_E130G | 11 | L39A_Y66F_R67L |
| 12 | V23M | 12 | K28V_N41D_R67H_V72M_E123K | 12 | W40E_E63A |
| 13 | A11S | 13 | T27S_N41D_Q55R | 13 | E63A |
| 14 | A7G_N41D_Q55R_I115S | 14 | N41D_F58L_V72M_*133Y | 14 | E63S_L68M |
| 15 | A7G_Q55R | 15 | N41D_V72M | 15 | V51*_E63K |
| 16 | I12F_N41D_Q55R_A117E | 16 | F58L_V72M | 16 | P62W_E63T |
| 17 | Q55R | 17 | I12F_N41D_Q55R_H60Y | 17 | P62Y_E63T |
| 18 | R85L | 18 | I12F_N41D | 18 | Q48G |
| 19 | Y100S | 19 | A7G_N41D_V52E_Q55R | 19 | V50I_K70R_E71I |
| | | 20 | N41D_G121R | 20 | Y49R |

In the same selection, N41D combined with Q55R, a double-mutant, was also identified. Simultaneously, V72M was selected as enriched. However, no clone was selected that contained combinations of these mutations, such as the triple mutant N411D, Q55R, V72M, which was thought to be due to the absence of all of the possible triple mutation combinations in the naïve library. Many mutations, such as V72M for example, have convincing computational data to rationalize their significance. All of this data was analyzed, and a second library was created in which the combination N411D, Q55R, V72M was purposely included in the library as well as many other rationally designed combinations. In the next round of selection using yeast display, this mutant was selected as one of the most enriched clones (Table 10, directed evolution round 2).

In parallel, one round of directed evolution was performed using SNAP display on a library designed to have targeted mutations based on computational design as well as some of the hits that had already been seen in the prior rounds of directed evolution. Each position was allowed to mutate to all 20 amino acids in different combinations. Some similar positions were identified in this selection as in other previous selections, and in some cases, the residue was mutated to a different amino acid than was seen previously. For example, E63 was identified in round 1 of directed evolution mutated to lysine (K), but in round 3 (Table 10, directed evolution round 3), it was mutated to alanine (A) or serine (S). In the fourth round of directed evolution, a library was created in which many of these combinations were tested, including N41D, Q55R, E63S, L68M, V72M, and additional residues were mutated to all 20 amino acids randomly (such as Y100). From this fourth round of directed evolution, the enriched clones listed in Table 9 were identified, and clones such as N41D, Q55R, E63S, L68M, V72M, Y100R were selected and chosen to be expressed in E. coli, purified, and tested for binding activity in a high throughput assay on the Octet platform.

Selected candidates from the fourth round of directed evolution, using SNAP display selection, as compared to candidates from other prior selections are shown in FIG. 12B. In the high-throughput assay, Octet sensors are coated with the peptide of interest and dipped in buffer containing the purified protein. The traces for the alanine peptide are shown in FIG. 12B. Improved binding is illustrated by an increase in the response based on a shift in wavelength, given in nm, over time (association curves between 0 and 200 see, dissociation curves between 200 and 500 see). It is possible to obtain an approximation of the relative binding by comparing the response after 200 seconds for each protein. Each protein has approximately the same molecular mass and is used at the same concentration, and ranking the proteins by response level in this assay provides an approximation of which proteins have improved binding. Studying the on and off-rate can also give insight into the mechanism of binding. The response values for different peptides with the constructs selected from this round of selection are given in Table 11, which shows responses (in nm) for each peptide with the two N-terminal residues of the peptide listed (AA, VA, LA, etc.) after 200 see incubation during the association phase of incubation with a mutant protein candidate.

TABLE 11
Binding affinity of selected candidates as compared to the
wild-type protein (PS557) in a high-throughput Octet assay.

| Protein | Mutations | AA | VA | LA | MA | IA | FA |
|---|---|---|---|---|---|---|---|
| PS960 | N41D_Q55R_H60I_V72M | 1.6787 | 9.2924 | 10.4523 | 6.8605 | 9.9776 | 0.0851 |
| PS961 | N41D_Q55R_E63S_L68M_V72M_Y100R | 4.8712 | 11.3444 | 11.7773 | 9.2586 | 11.4695 | 2.0502 |
| PS962 | N41D_Q55R_P62Y_E63A_V72M_Y100R | 3.7624 | 10.6694 | 10.483 | 8.367 | 10.7342 | 2.5726 |
| PS963 | N41D_Q55R_E63A_V72M_Y100R | 4.0599 | 10.4034 | 9.6473 | 8.3995 | 10.207 | 2.2496 |
| PS557 | WT | 0.6115 | 4.8006 | 7.1558 | 1.948 | 7.2041 | 0.6341 |

### Example 5. Development of Arginine Recognizer PS1122

This example describes the development of PS1122, an engineered variant of a UBR protein from Kluyveromyces marxianus (PS621) with increased affinity for arginine and histidine that exhibits improved recognition of arginine on-chip. Based on analysis of binding kinetics and on-chip results, PS1122 has ˜7-fold higher binding affinity for N-terminal arginine than PS621, resulting in a favorable increase in pulse duration for RX dipeptides and faster binding. These properties combine to improve the recognition range for arginine tripeptides and the accuracy of ROI detection. It is estimated that PS1122 can visibly detect ˜52% of all arginine positions in the human proteome, which equals 2.9% of the total proteome (an increase from ˜1.4% with PS691).

### Directed Evolution Approach

PS621 (and its tandem version PS691) binds to arginine (R), histidine (H), and lysine (K). Observable binding of PS621 to R on-chip is limited to −25% of arginine positions in the proteome due to the influence of downstream residues on pulse duration. Directed evolution was used to select for variants of PS621 with stronger arginine binding. Multiple types of variant libraries were subjected to many cycles of selection and mutational evolution to arrive at a panel of candidate recognizer variants (PS1101-PS1122) that were carried forward for biochemical and single-molecule investigation.

### Octet Analysis

The variant binders (PS1101-PS1122) and controls were expressed in E. coli, purified in a high-throughput workflow, and evaluated for binding to N-terminal amino-acids on the Octet platform. The peptides used in the assay mostly contained a penultimate alanine and consisted of the sequence XAKLDEESILKQK (SEQ ID NO: 1074). Peptides of the sequence RXKLDEESILKQK (SEQ ID NO: 1076) were also used to evaluate the penultimate residue effect. The set of Octet response measurements for RX (various R dipeptides), HA and KA is summarized in Table 12.

TABLE 12
Octet response for PS621 variant binders measured with
RX (various R dipeptides), HA and KA peptides at 30° C.

| Binder | RA | RL | HA | KA | RE | RQ | RS | RR | RF |
|---|---|---|---|---|---|---|---|---|---|
| PS1101 | 6.5 | 7.7 | 0.1 | 1.4 | 1.9 | 4.7 | 4.3 | 2.8 | 6.0 |
| PS1102 | 8.6 | 10.4 | 2.5 | 6.6 | 6.6 | 9.3 | 8.5 | 7.0 | 9.3 |
| PS1103 | 6.2 | 8.4 | 0.1 | 2.9 | | | | | |
| PS1104 | 9.4 | 11.3 | 3.4 | 7.3 | 7.6 | 9.7 | 8.9 | 8.0 | 10.5 |
| PS1105 | 6.5 | 7.5 | 0.2 | 2.6 | 2.9 | 5.9 | 5.5 | 4.4 | 7.2 |
| PS1106 | 7.6 | 9.0 | 0.6 | 4.7 | | | | | |
| PS1107 | 7.8 | 8.1 | 0.6 | 3.9 | | | | | |
| PS1108 | 8.2 | 10.3 | 1.2 | 5.0 | | | | | |
| PS1109 | 8.2 | 10.5 | 0.6 | 4.7 | | | | | |
| PS1110 | 7.0 | 9.8 | 2.4 | 6.6 | | | | | |
| PS1111 | 7.9 | 10.0 | 0.1 | 2.7 | | | | | |
| PS1112 | 8.6 | 9.9 | 0.1 | 2.3 | | | | | |
| PS1113 | 7.7 | 9.3 | 0.2 | 3.8 | | | | | |
| PS1114 | 9.2 | 9.7 | 1.2 | 5.3 | | | | | |
| PS1115 | 10.8 | 11.8 | 3.0 | 6.5 | 7.6 | 9.1 | 8.5 | 6.7 | 10.0 |
| PS1116 | 7.1 | 8.5 | 0.1 | 1.2 | | | | | |
| PS1117 | 8.6 | 9.0 | 0.5 | 3.5 | | | | | |
| PS1118 | 9.8 | 10.6 | 1.8 | 5.0 | | | | | |
| PS1119 | 8.6 | 9.6 | 0.7 | 3.3 | | | | | |
| PS1120 | 9.9 | 11.7 | 1.7 | 5.5 | 8.8 | 9.5 | 8.3 | 5.9 | 10.2 |
| PS1121 | 9.5 | 10.6 | 2.2 | 5.4 | | | | | |
| PS1122 | 9.9 | 11.2 | 1.3 | 4.6 | | | | | |
| PS621 | 9.3 | 10.2 | 2.0 | 5.2 | 5.8 | 7.4 | 7.1 | 6.3 | 8.4 |

### Binding Affinity by Polarization

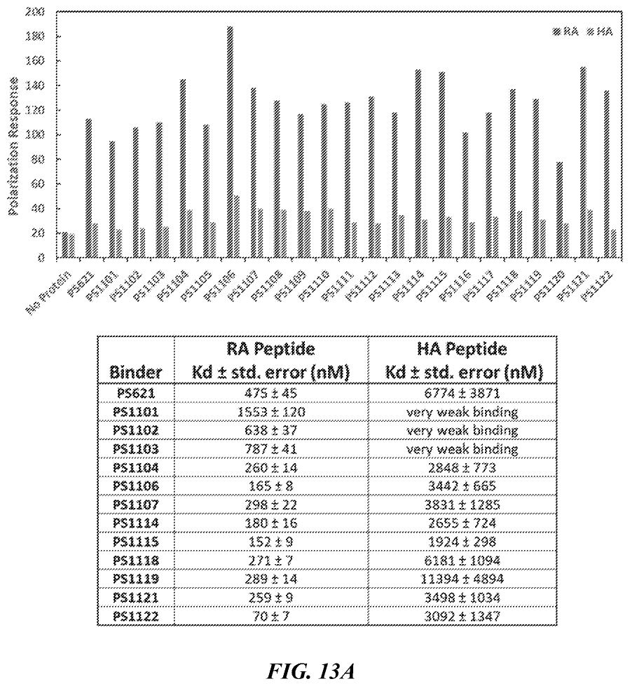

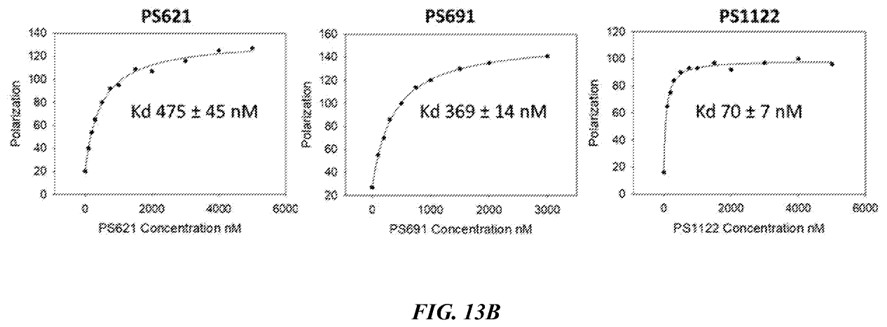

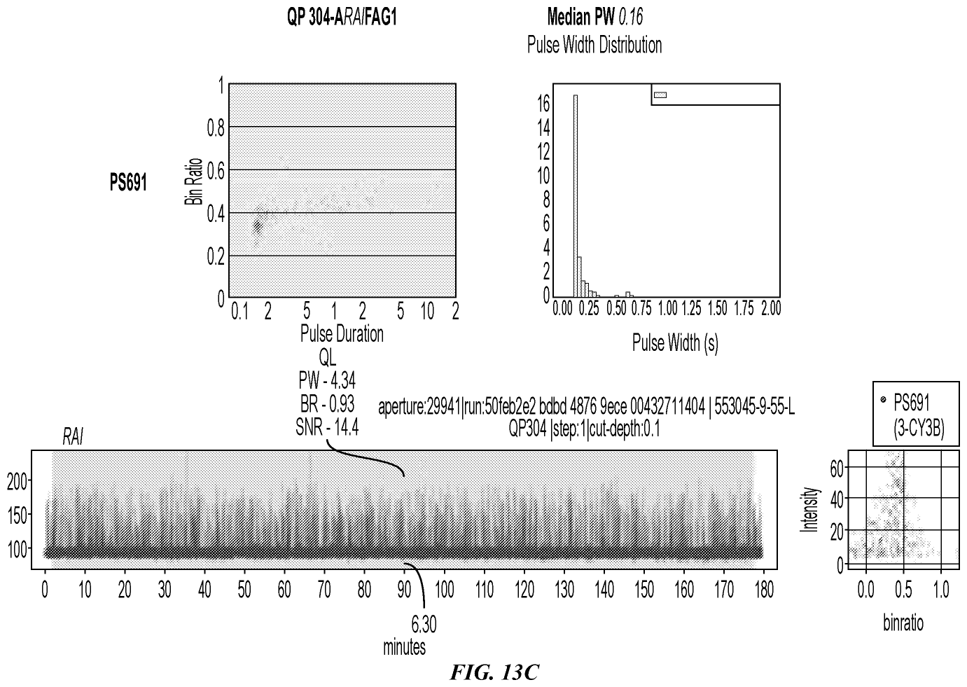

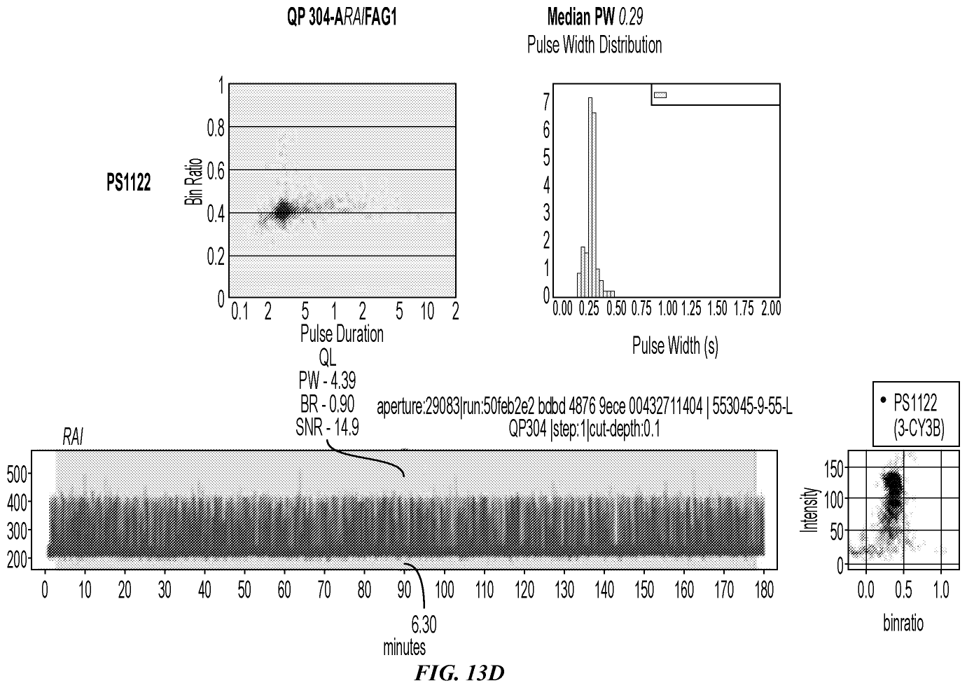

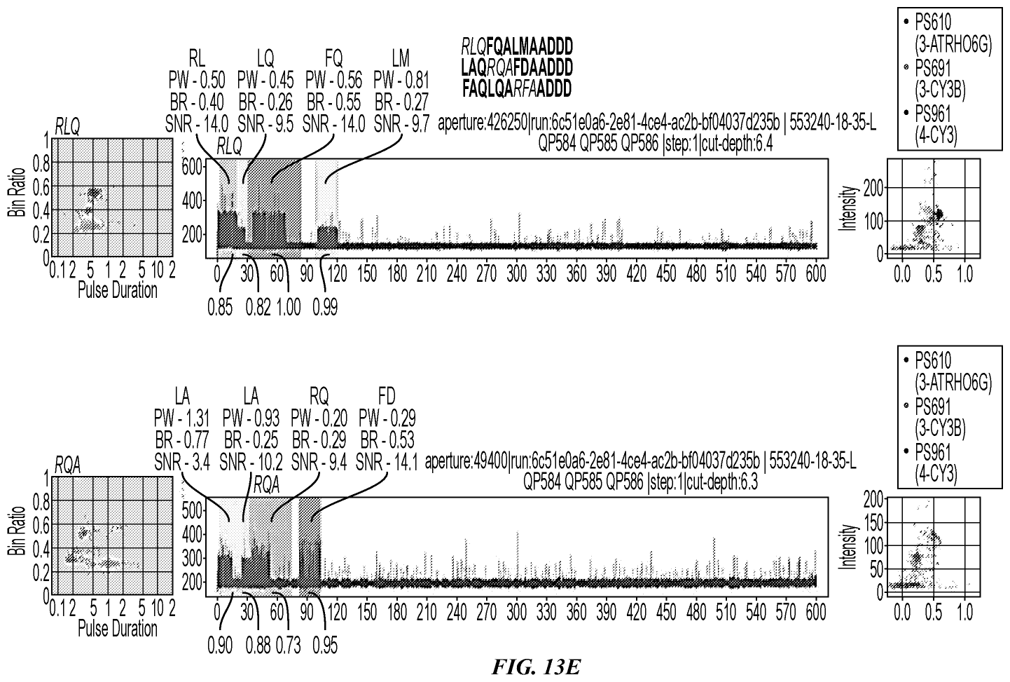

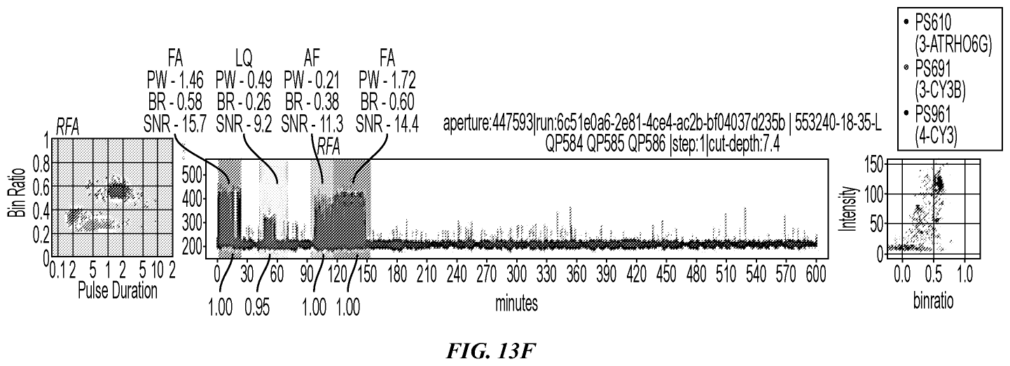

Fluorescence polarization assays were performed with all candidates, and single point binding responses were measured at a fixed concentration of the binders (FIG. 13A). This assay measures the strength of the interaction between a binder and a labeled peptide (XAKLDEESILKQK-FITC (SEQ ID NO: 1074)). Based on the binding responses, we further investigated select candidates by measuring their Kds. We carried out multiple polarization binding titration analysis at increasing concentration of binder protein, and the Kds were determined from the titration curves (FIG. 13B).

PS1122, PS1115, PS1106, PS1114, PS1121 and PS1104 showed the most improvement in binding for RA peptide. Multiple variants also showed improvement in HA binding in comparison with PS621. RA binding affinity determination titration curves for PS621, PS691, and PS1122 are shown in FIG. 13B. In comparison with PS621 and PS691, PS1122 displayed a 5-fold to 7-fold increase in binding affinity for RA peptide and ˜2-fold increase in binding affinity for HA peptide.

Stopped-Flow Rapid Kinetic Analysis for Kₒₙ and K_{off}

The on-rate constant (kₒₙ) and the dissociation rate (k_{off}) were measured for PS621 and variants for RA and HA peptides using stopped-flow assays (results summarized in Table 13). These measurements were performed to predict relative improvements in pulse duration and interpulse duration on-chip.

TABLE 13
Kₒₙ rate constants and k_{off} rates derived for PS621
variants by the stopped-flow instrument with rapid
kinetic method (dash indicates not measured).

| | RA Peptide | RA Peptide | RL Peptide | HL Peptide |
|---|---|---|---|---|
| Binder | (kon/nM/s) | (koff/s) | (koff/s) | (koff/s) |
| PS621 | — | 61.78 | 17.742 | — |
| PS691 | 0.026 | 69.351 | 19.742 | 51.418 |
| PS1115 | — | — | 14.405 | 40.195 |
| PS1122 | 0.019 | 21.306 | 5.5171 | 26.917 |

In these assays, RX peptides gave a better signal and accurate measurements due to tighter binding than HA. The PS1122 kₒₙ rate constant was comparable to the tandem binder PS691. The k_{off} rate for PS1122 for RA and RL peptide was ˜3-3.5 fold slower than PS621 or PS691, which predicted longer pulse duration on-chip. PS1122 was also ˜2-fold slower in dissociating from HA peptide than PS691. These measurements identified PS1122 as a variant to evaluate in single molecule assays.

In parallel, to evaluate multiple PS621 variants on-chip, a next-generation on-chip recognizer screening method was employed. PS621 variants were purified in biotinylated form, labeled in micro-scale using a modified protocol with streptavidin-tetraCy3B, and recognition runs were performed for select candidates. Various RX penultimate peptides were used on-chip to evaluate the improvement in R recognition coverage for PS1122.

### On-Chip Recognition of RA Dipeptide

For in-depth on-chip characterization, PS1122 was purified and labelled in large scale, using streptavidin-tetraCy3 dye complex for labelling. Ensemble and screening on-chip assays indicated that binding of PS1122 to arginine is improved enough to recognize RA dipeptide on-chip. Analysis of recognition run using the QP304-RAIFAG peptide confirmed the presence of visible binding of PS1122 to N-terminal RA with a longer pulse duration (0.29 s) than PS691 (0.16 s) (FIGS. 13C-13D).

Sequencing Performance and Arginine Tripeptide Coverage with PS1122

Sequencing performance of PS1122 and PS691 were compared using QP433 (RLIFAYP (SEQ ID NO: 1087)) and other peptides. Multiple multiplexed dynamic assays were performed using PS1122 to further evaluate its range of arginine recognition for RXA tripeptides (FIGS. 13E-13F).

Using the determined arginine tripeptide pulse duration data from the multiplexed runs with PS1122, predicted pulse durations were determined for all 400 RXX tripeptides, and the arginine proteome coverage for PS1122 was estimated (FIG. 10G). The results predict coverage of 52% of arginine positions, representing 2.9% of the human proteome.

### Example 6. PS961 Engineered Peptide Binding Enhancements

PS961 features six-point mutations on top of the PS557 precursor (N41D, Q55R, E63S, L68M, V72M, Y100R) and binds with better affinity or on chip performance to L/I/V/A/M/P N-terminal peptides than PS557. Each point mutation was analyzed in the context of the protein complexed with an alanine tripeptide and offer structure-based rationalizations for the selection of these substitutions in the directed evolution screens over the native PS557 amino acid identities.

### Direct Enhancements to the Binding Pocket

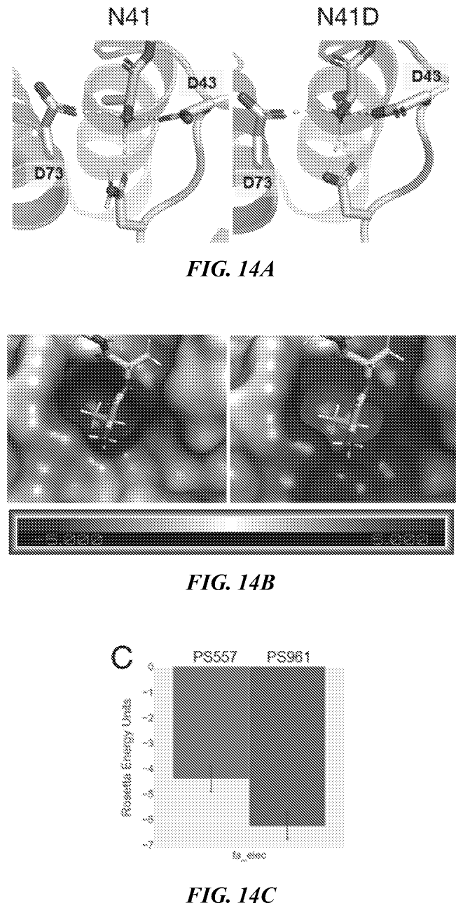

Exchanging asparagine for an aspartate in the outer ring of the binding pocket at position 41 enhances both long- and short-range interactions of PS961 with an N-terminal peptide ligand (FIG. 14A).

Long-range electrostatic interactions increase the probability that the positively charged peptide N-terminus interacts with the protein, and the additional negative charge from the aspartate side chain enhances electrostatic steering into the binding site compared to the asparagine side chain, as shown in the electrostatic surface charge distribution (FIG. 14B).

This amino acid position also sits directly in the triad of residues integral for binding, as these form short-range electrostatic interactions with the N-terminal amino group of the peptide. An aspartate at position 41 with two electronegative atoms on either side of the side chain can interact more favorably with the peptide via lowering the conformational entropy of a significant electrostatic interaction in the binding pocket. Also, the side chain of the aspartate is expected to bear a more electro-negative polarization of the orbital which can enable a stronger hydrogen bond between PS961 and the peptide N-terminus, compared to the partially charged side chain of asparagine. The Rosetta all-atom energy function corroborates the presence of this stronger interaction by quantitatively identifying lower and more favorable Coulombic energy (the “fa_elec” term in the score function) that involve the N-terminal amino acid in PS961 than when it is in the presence of the asparagine-bearing triad (FIG. 14C).

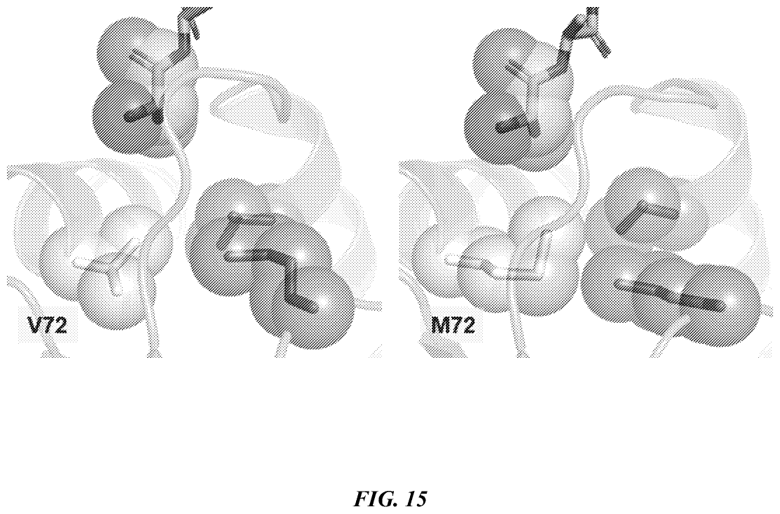

A valine-to-methionine substitution at position 72 introduces a longer non-polar side chain into the PS961 binding pocket (FIG. 15). These extra non-polar atoms can intercalate further into the cavity, thus decreasing the pocket volume and increasing hydrophobic interactions with smaller N-terminal amino acids.

### Optimization of Non-Pocket Hydrophobic Packing

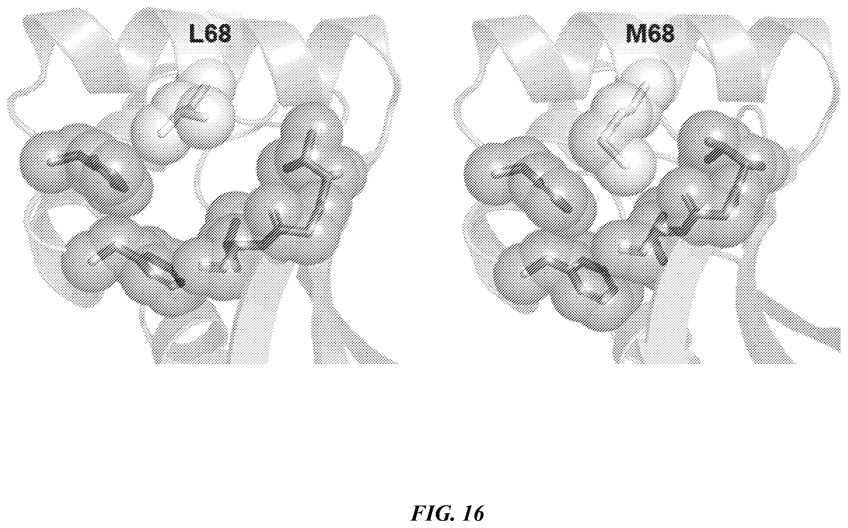

As methionine has a longer non-polar side chain than leucine, this mutation at position 68 can interact favorably with amino acids on a neighboring beta sheet such that a structural cavity is often filled in simulations (FIG. 16). Tightly packed non-polar amino acid side chains inside the core of a protein reduce the unfavorable conformational entropy of internal cavities and increase favorable hydrophobic interactions, which together increase the stability of the overall fold and likely decrease the cost in energy to form the binding pocket prior to binding.

### Mitigation of Potential Alternative Binding Site and Increase in Surface and Net Charge

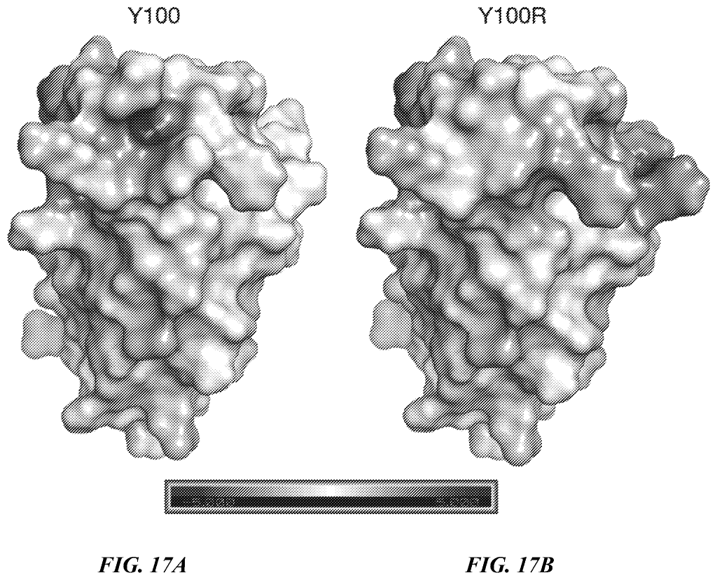

As the N-terminus of the peptide ligand bears a permanent positive charge, any negatively charged pockets that exist naturally on the surface of PS557 could interfere with the probability of a productive protein-peptide interaction in the binding site by acting as an alternative lower-affinity competitive binding site. The mutation Y100R mitigates a potential off-pocket interaction by reducing negative surface charges present in the absence of the arginine mutation (FIGS. 17A-17B). Additionally, in conjunction with Q55R and E63S, the overall surface and net charge of the protein is increased, more directly favoring intended binding events for proper on-chip recognition.

Increased Probability of Loop Conformations that Positively Interact with the Peptide

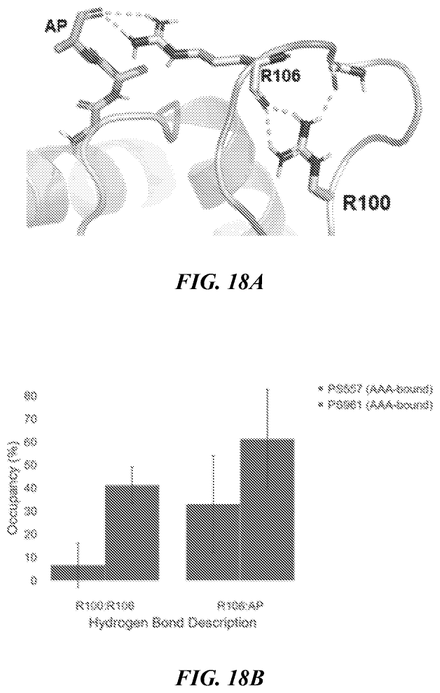

Molecular dynamics simulations of PS961 and PS557 bound to an AAA-tripeptide reveal an enhanced potential for an arginine side chain at position 100 to hydrogen bond to neighboring loop residues compared to the tyrosine in PS557. This arginine is often involved in a complex hydrogen bond network involving R106 and the backbone carbonyl of the antepenultimate residue of the peptide, lending extra stabilization to the bound form of the peptide (FIG. 18A). The average occupancy of the R100:R106 hydrogen bond, calculated as the percentage of 1,000 simulation frames that realize a particular interaction, is roughly six-fold higher in simulations of PS961 bound to an AAA-tripeptide compared to PS557 (FIG. 18B). In turn, the R106 antepenultimate hydrogen bond is more likely to occur in PS961 than PS557.

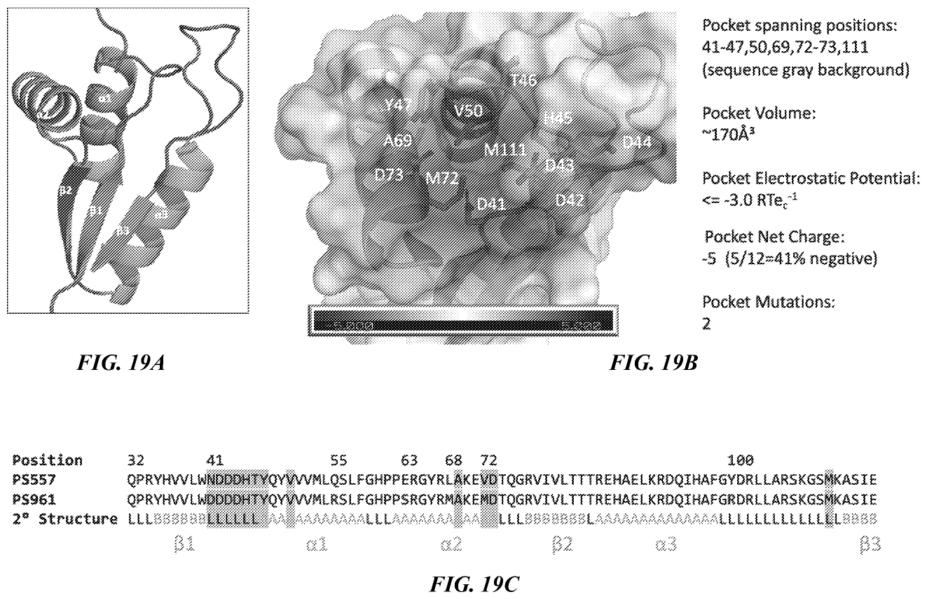

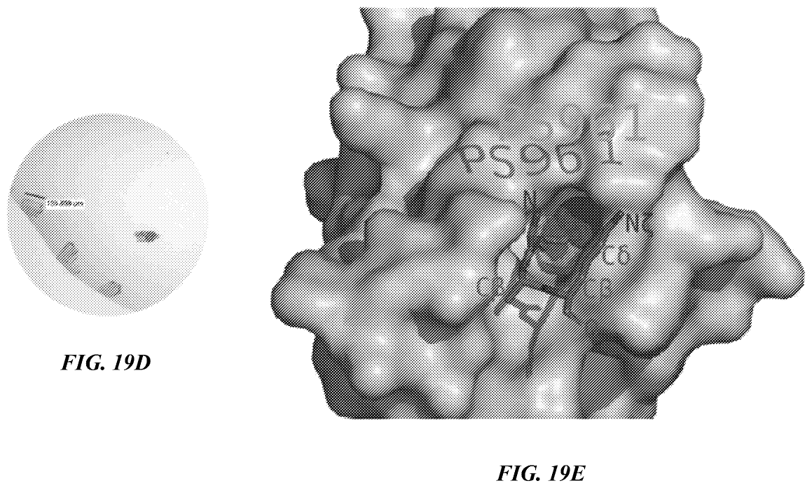

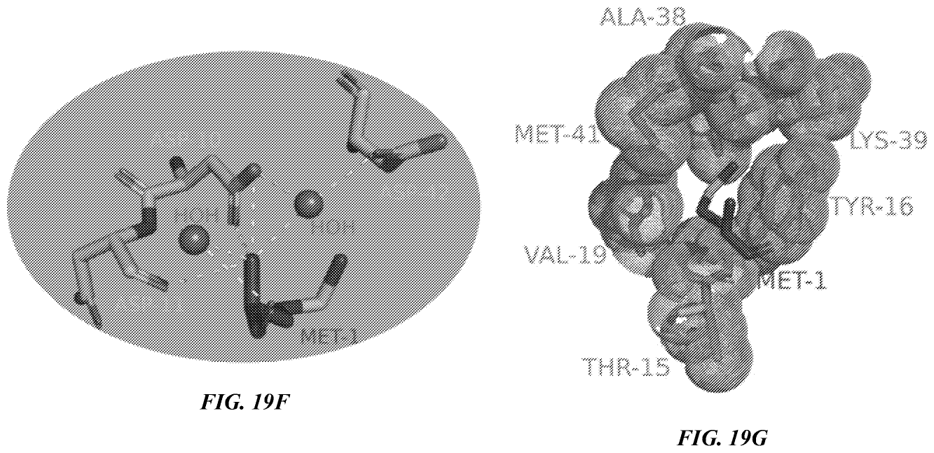

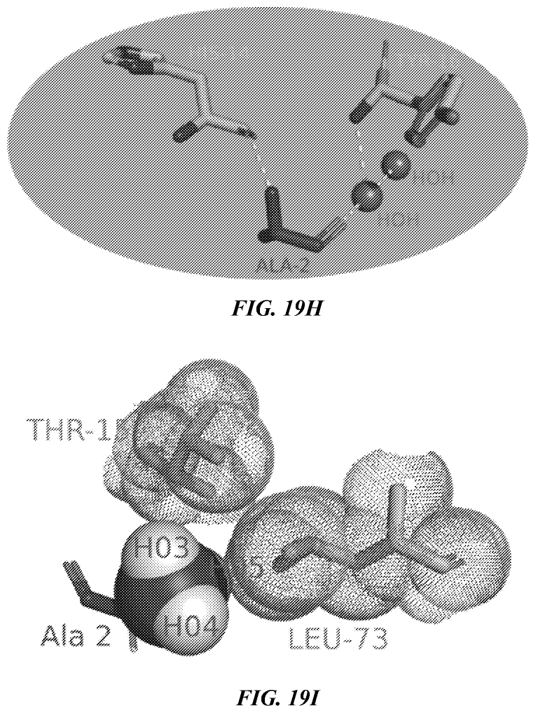

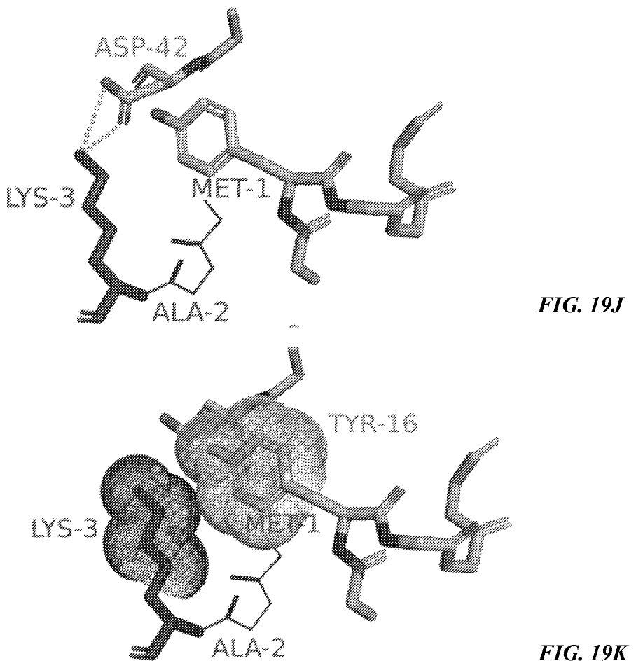

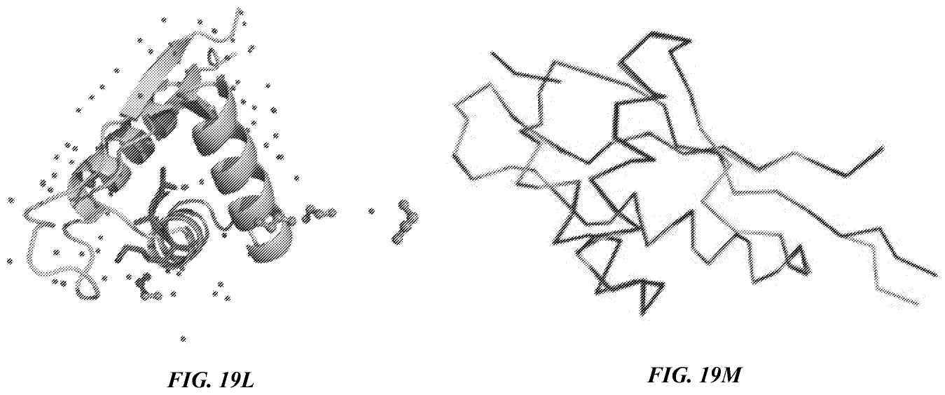

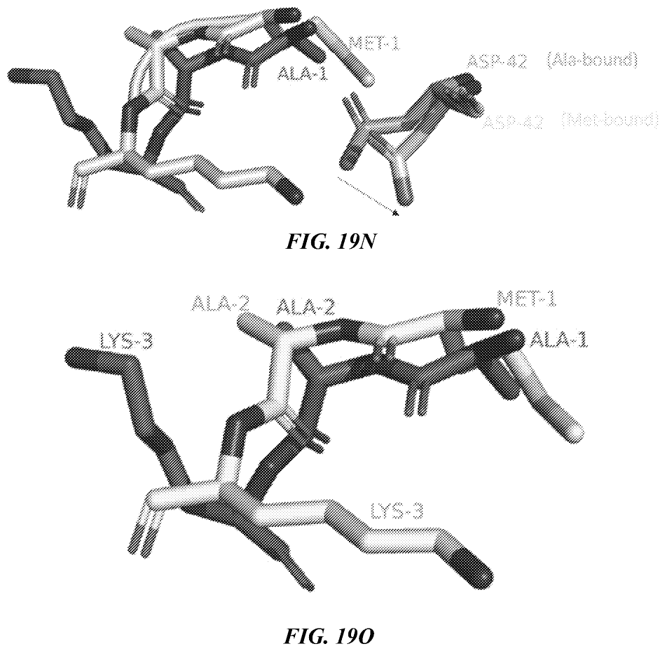

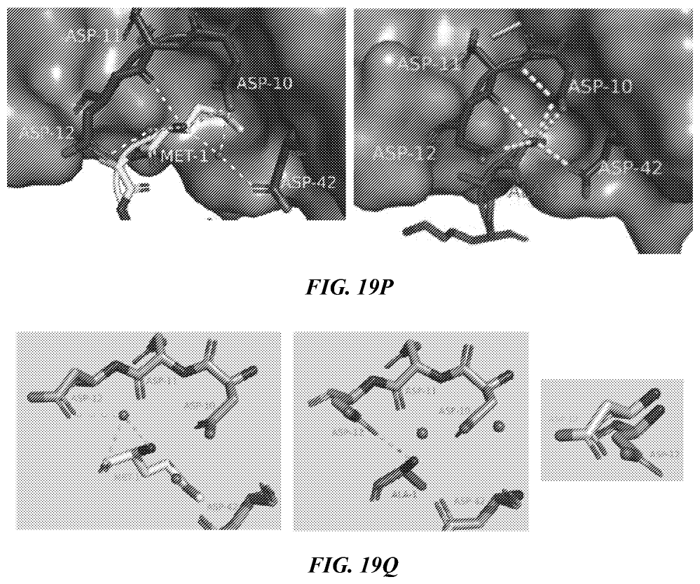

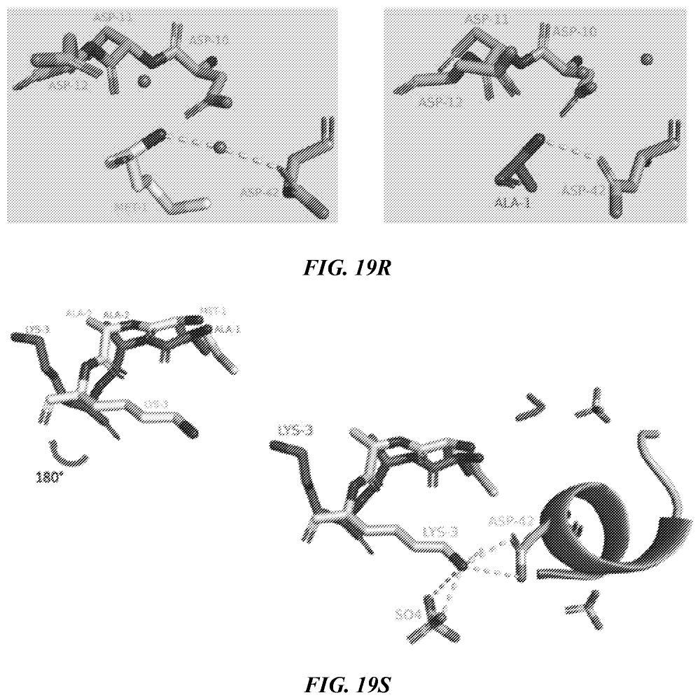

FIGS. 19A-19C show secondary structure, sequence, and binding pocket properties of PS961. FIG. 19A shows a classification of the protein into secondary structure groups. FIG. 19B shows Poisson Boltzmann electrostatic potential surface map of binding pocket with residues labeled that form the binding pocket, with corresponding pocket properties listed. FIG. 19C shows the sequence of natural parent protein and engineered variant highlighting mutations, pocket positions and secondary structure assignment per position.

### PS961 Crystallography

The crystal structure of PS961 in complex with a target peptide having N-terminal methionine (Met-Ala-Lys-Leu (MAKL) (SEQ ID NO: 1047)) was resolved. The protein:peptide complex was generated and purified, and diffracting crystals of PS961:MAKL were obtained (FIG. 19D). A complete X-ray dataset from these crystals was collected, and the final structure was solved and refined at 1.9 Å resolution (FIG. 19E). FIGS. 19F-19K show how PS961 binds the target peptide MAKL (SEQ ID NO: 1047).

FIG. 19F shows that the Met in the target peptide is contacting Asp10 and Asp11 in recognizer PS961. The crystal structure also shows interactions with two water molecules (spheres), one of which is held at the proper orientation by Asp42 of the recognizer. Substitution of Asp42 might alter the binding affinity of this N-terminal residue in the peptide. In addition to the ionic interactions shown in FIG. 19F, Met1 in the peptide also makes non-polar contacts with at least six residues in PS961 (FIG. 19G, labeled residues other than “MET-1”). These residues in PS961 are positioned around the sidechain of Met. With the proper substitution of some of these residues, other N-terminal amino acids, different from Met, can potentially be recognized and bound by PS961.

The next residue in the peptide, Ala2, contacts the backbone of His14 in PS961 as it also H-bonds with a water molecule (FIG. 19H, sphere), which in turn contacts Tyr16 and an additional water molecule. It follows that replacement of Tyr 16 might compromise the presence of said water molecule, altering the binding of this penultimate residue in the peptide. The crystal structure also shows that the oily sidechain of Ala2 in the peptide (with three methyl protons) is lodged between residues Thr15 and Leu73 in the recognizer (FIG. 19I), which opens the possibility of replacing these residues in the recognizer with smaller sidechains to make space for larger sidechains in the peptide. Lys3 in the peptide forms a salt bridge with Asp42 in the recognizer (FIG. 19J, ionic interaction illustrated by dashes). Lys3 in the peptide is mostly removed from the binding site; however Tyr16 in the recognizer is at a very short distance as indicated by their surfaces (FIG. 19K).

The crystal structure of PS961 in complex with a target peptide having N-terminal alanine (Ala-Ala-Lys-Leu (AAKL) (SEQ ID NO: 1048)) was resolved. The protein:peptide complex was generated and purified, and diffracting crystals of PS961:AAKL were obtained. A complete X-ray dataset from these crystals was collected, and the final structure was solved and refined at 1.39 Å resolution. FIG. 19L shows a panoramic view of the PS961:AAKL complex structure, with PS961 shown in cartoon representation, AAKL (SEQ ID NO: 1048) peptide shown in sticks, water molecules displayed as spheres, and PEG molecules shown in stick-and-ball representation.

Because of the high resolution, the PS961:AAKL crystal structure shown in FIG. 19L shows many water molecules that could be modelled. It also shows alternate conformations for three residues in the recognizer. The recognizer in the AAKL (SEQ ID NO: 1048) complex has overall the same fold that was observed in the PS961:MAKL complex. Thus, the backbone of the recognizer in both structures can be superimposed with an average deviation of only 0.23 Å. FIG. 19M shows a backbone superimposition of PS961 when bound to the Met peptide and to the Ala peptide.

Based on a comparison of the different PS961 complexes, some sidechain changes in PS961 are evident. Since the AAKL (SEQ ID NO: 1048) and MAKL (SEQ ID NO: 1047) peptides differ only in their first residue, the binding pocket for these residues was evaluated. Because the sidechain of Ala1 in the AAKL (SEQ ID NO: 1048) peptide is smaller than Met1 in MAKL (SEQ ID NO: 1047), the recognizer has rearranged some of its residues to make space for Met (FIG. 19N). When bound to the AAKL (SEQ ID NO: 1048), the side chain of Aps42 is pointing towards the binding pocket, but when bound to the MAKL (SEQ ID NO: 1047) peptide, this same sidechain has been displaced by 1.6 Å. FIG. 19N shows displacement of the Asp42 sidechain in the recognizer when comparing binding to AAKL (SEQ ID NO: 1048) and to MAKL (SEQ ID NO: 1047). The arrow indicates how the Asp42 sidechain moved to engage with the Met1 of the MAKL peptide.

In general, both peptides (AAKL (SEQ ID NO: 1048) and MAKL (SEQ ID NO: 1047)) had the same orientation and, as expected, the terminal amino group (NH2) in both peptides adopts approximately the same configuration. FIG. 19O shows a comparison of the AAKL (SEQ ID NO: 1048) (bottom sticks) and MAKL (SEQ ID NO: 1047) (top sticks) peptides when bound to PS961. The backbone of both peptides follows the same trace, but an overall displacement of 1.4 Å from each other was observed. Of note, the main difference between the peptides is the configuration of the sidechain of Lys3. Additionally, the fourth residue of the peptide could not be observed (Leu4), which was also the case for the PS961:MAKL complex. Most of the binding interactions are clustered in the first residue of the peptide, some in the second, and almost none in the third position, suggesting that certain downstream residues are more free and disordered.

Binding of the N-terminal residue in each structure was evaluated. The terminal amino group (NH2) of AAKL (SEQ ID NO: 1048) contacts the same PS961-residues as MAKL (SEQ ID NO: 1047), that is, Asp10, Asp11, Asp12 and Asp42. However, Asp12 and Asp42 have reoriented their sidechains, and the way these residues engage the peptides is also different. FIG. 19P shows a comparison of the interaction of the AAKL (SEQ ID NO: 1048) (left panel) and MAKL (SEQ ID NO: 1047) (right panel) peptides with residues in PS961. Peptides are shown in sticks (MAKL (SEQ ID NO: 1047) and AAKL (SEQ ID NO: 1048)), recognizer PS961 is shown in surface and stick representation, and water molecules can be seen as spheres mediating interactions in the PS961:MAKL complex (left panel).

In the MAKL (SEQ ID NO: 1047) structure, Asp12 in the recognizer contacts the peptide NH2 group indirectly, via a water molecule (FIG. 19Q, left panel). In the AAKL (SEQ ID NO: 1048) structure however, the carboxyl group of Aps12 directly hydrogen-bonds with the NH2 group in the peptide (FIG. 19Q, central panel). When the conformations of the recognizer Asp12 are superimposed, it is clear that its side chain has reoriented (FIG. 19Q, right panel).

Another novel interaction observed in the PS961:AAKL structure is the hydrogen bond between the terminal amino group of the AAKL (SEQ ID NO: 1048) peptide and the sidechain carboxylate of Asp42 in the recognizer (FIG. 19R). This binding interaction is a direct contact between peptide and recognizer, whereas the one observed in the PS961:MAKL crystal structure was indirect, mediated by a water molecule.

The most evident structural difference between the peptides, when superimposed, is the opposite orientations of the Lys3 sidechain. FIG. 19S shows a superimposition of the AAKL (SEQ ID NO: 1048) and MAKL (SEQ ID NO: 1047) peptides showing the 1800 flip of the LYS sidechain in the third position (left), and different orientations of the Lys3 sidechain in the MAKL (SEQ ID NO: 1047) and AAKL (SEQ ID NO: 1048) peptides when bound to PS961 (right). In the AAKL (SEQ ID NO: 1048) peptide, Lys3 doesn't make any polar contacts, and its sidechain is facing away from the binding pocket. In the MAKL (SEQ ID NO: 1047) peptide, the ε-amino group of Lys3 is contacting both Asp42 in the recognizer and a sulfate ion in the solvent. These interactions appear to hold Lys3 in the observed orientation.

### Example 7. PS1122 Engineered Peptide Binding Enhancements

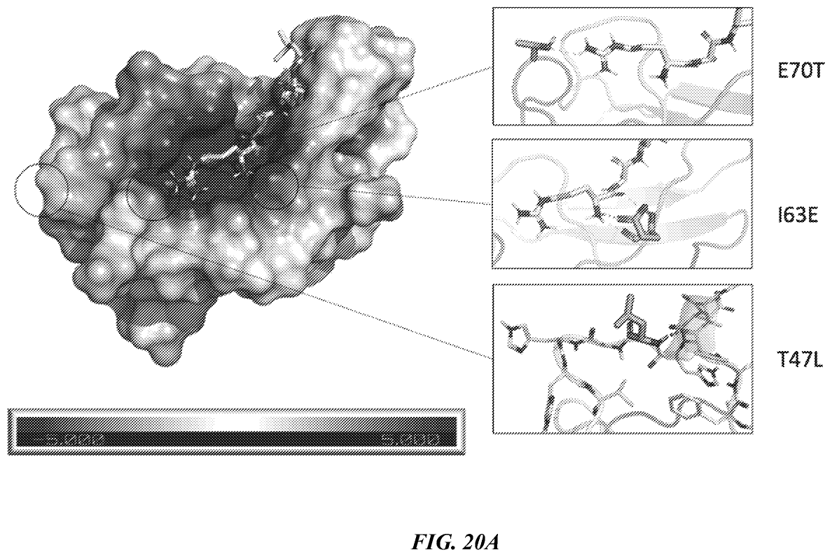

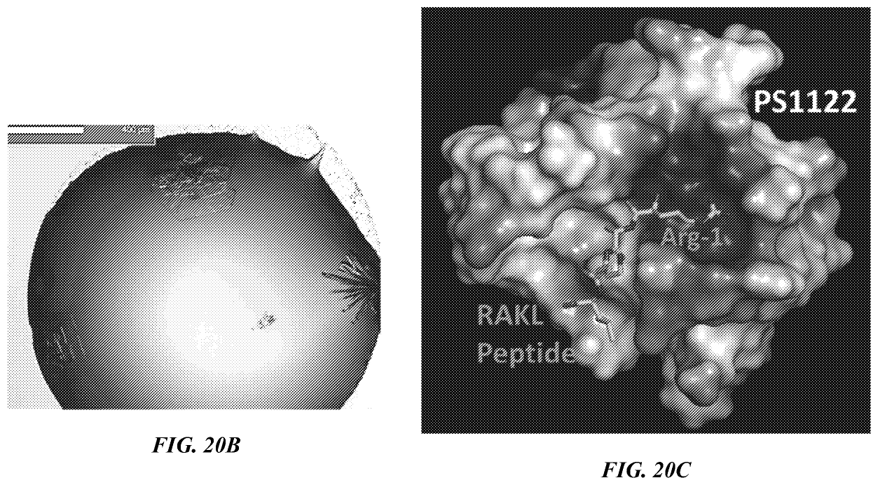

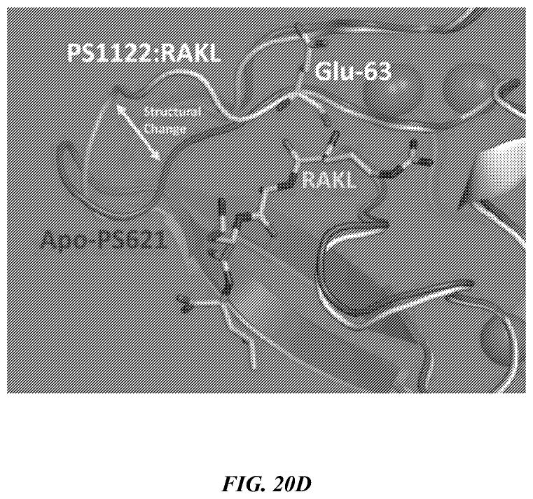

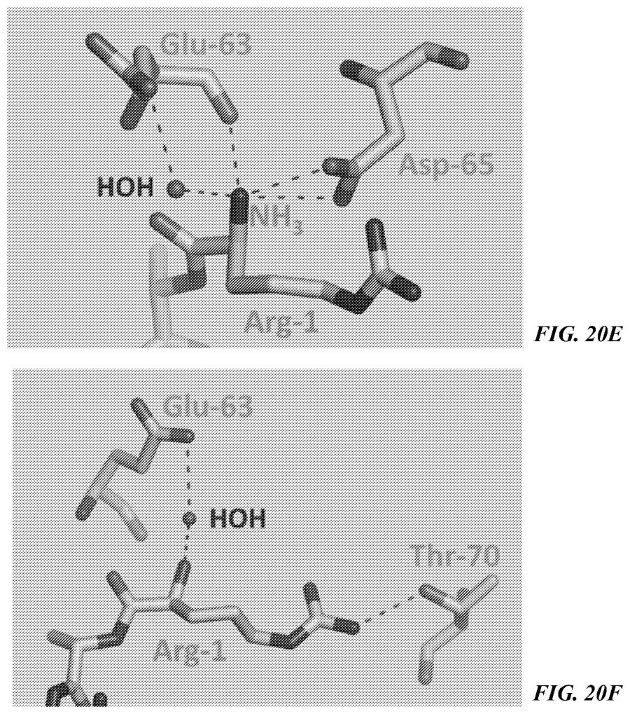

Modeling of mutations from directed evolution selections revealed two of the three mutations (E70T, 163E) contributed one new hydrogen bond each to the N-terminal arginine (FIG. 20A). In the absence of a hydrogen bond acceptor at these positions, the arginine most likely continues to transiently hydrogen bond with water which results in no gain in energy upon peptide binding. However, if two new stable hydrogen bonds form upon binding, where each hydrogen bond can result in a gain of energy equivalent to a few kilocalories on average, an order of magnitude change in the Kd is expected and likely explains the improved binding.

T47 L is a mutation that came up significantly enriched in directed evolution selections across numerous peptides and does not appear to have a direct impact on binding based on structural analysis. It occurs in a helical region of the structure near the metal binding sites, which can have a significant impact on stabilization of the protein overall, across numerous functional and non-functional conformations of the structure.

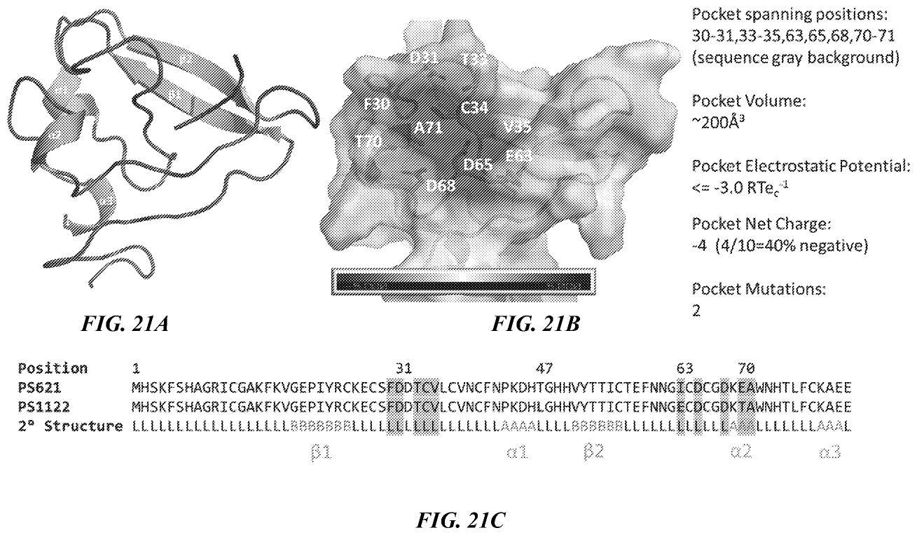

FIGS. 21A-21C show secondary structure, sequence, and binding pocket properties of PS1122. FIG. 21A shows a classification of the protein into secondary structure groups. FIG. 21B shows Poisson Boltzmann electrostatic potential surface map of binding pocket with residues labeled that form the binding pocket, with corresponding pocket properties listed. FIG. 21C shows the sequence of natural parent protein and engineered variant highlighting mutations, pocket positions and secondary structure assignment per position.

### PS1122 Crystallography

The crystal structure of PS1122 in complex with a target peptide having N-terminal arginine (Arg-Ala-Lys-Leu (RAKL) (SEQ ID NO: 1049)) was resolved. The protein:peptide complex was generated and purified, and diffracting crystals of PS1122:RAKL were obtained (FIG. 20B). A complete X-ray dataset from these crystals was collected, and the final structure was solved and refined at 1.87 Å resolution.

The structure resulting from these crystals allowed for the visualization of all 4 amino acids of the RAKL peptide that bound to the PS1122 recognizer before crystallizing it. This allowed for the visualization of how recognizer PS1122 recognizes arginine amino acids in sequencing assays. For instance, the structure reveals that PS1122 utilizes a negatively charged pocket to recognize the positively charged arginine amino acid. FIG. 20C shows the crystal structure of recognizer PS1122 and bound RAKL (SEQ ID NO: 1049) peptide (sticks). Regions of PS1122 that have negative electrostatic potential are shown. Based on the structure, PS1122 uses a negatively charged pocket to recognize and bind arginine amino acids, which are positively charged.

The structure also shows the loop region between amino acids 57 and 62 of PS1122 adopt a new structure compared to the structure of PS621, which was determined without any bound peptide (FIG. 20D). FIG. 20D shows that a portion of PS1122 is different than the analogous portion observed in PS621 (the PS1122 predecessor). The loop to the left of amino acid Glu-63 (top-most loop) takes on a new shape compared to PS621 (bottom-most loop). This structural difference may be due to the new Glu-63 residue found in PS1122 or due to the binding of peptide compared to PS621.

The crystal structure also reveals how the terminal NH3 group of the peptide, which are unique to the first amino acids of peptides (Arg-1 in this case) is bound and recognized (FIG. 20E). FIG. 20E shows the NH3 group of the first amino acid of Arg-1 of the bound peptide interact with amino acids Glu-63, Asp-65, and a water molecule that is held in place by Glu-63 (interactions are depicted as dashed lines). Each hydrogen atom of this NH3 group makes a strong hydrogen bonding interaction with the Glu-63 and Asp-65 amino acids of PS1122. One of these interactions involves a water molecule that is also interacting with Glu-63.

The crystal structure of PS1122:RAKL rationalizes improvements over its PS621 predecessor. Two new amino acids E63 and T70 were designed into the new PS1122 sequence. Interestingly, both of these new residues make interactions with the arginine peptide that could not have been made by the PS621 recognizer (FIG. 20F). Thr-70 interacts with Arg-1 of the peptide directly, whereas the new side chain of Glu-63 interacts with Arg-1 through a well-positioned water molecule. FIG. 20F shows that the side chains of Glu-63 and Thr-70 of PS1122 both make interactions with the Arg-1 of the bound peptide, which was an important observation as both Glu-63 and Thr-70 were engineered into PS1122 so that PS1122 would bind arginine peptides better than previous arginine recognizers (interactions are depicted as dashed lines).

### Example 8. Ntaq1-Homologous Recognizer (PS1259): Engineered Deactivation of Catalysis and Peptide Binding Enhancements

A cysteine to serine point mutation in the catalytic triad of Ntals, a class of N-terminal amidases that convert Glutamine and Asparagine to Glutamate and Aspartate, respectively, has been shown to enable binding of their target N-terminal residues with micromolar affinity. However, this micromolar affinity is outside the range of binding needed for on-chip recognition.

A subclass of Amidases termed Glutaminases, which convert Glutamine to Glutamate, was identified and evaluated. A Glutaminase was computationally modeled with an analogous cysteine to serine mutation in the enzyme active site (C25S), which was estimated to confer sub-micromolar affinity for Glutamine. Additionally, another mutation in the catalytic triad (H78Q) was modeled and identified which improved binding further for Glutamine and Asparagine, like the related class of Amidases.

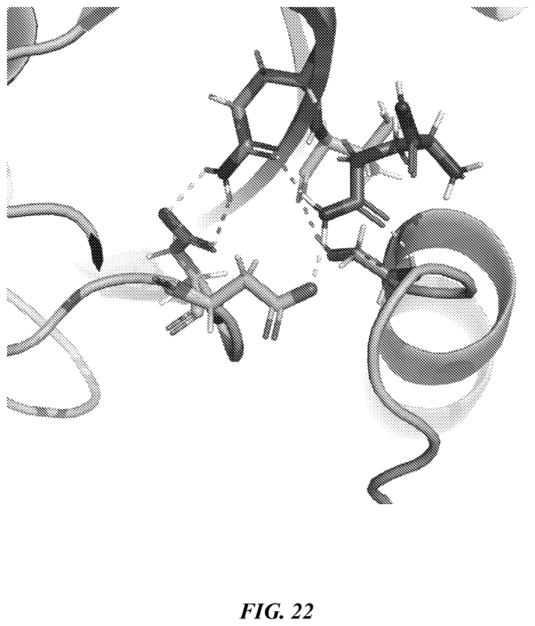

The computational modeling was confirmed experimentally, with PS1259 (Glutaminase from Scleropages formosus, the Asian Arowana fish, with two mutations) validated as a recognizer that shows detectable pulsing of Glutamine and Asparagine on chip, while requiring only computational screening to identify the homolog and model an improvement to the binding. FIG. 22 shows an alphafold model of PS1259 with network of hydrogen bonds enabled by two mutations. N-terminal Glutamine bound is shown in sticks, and hydrogen bonding with the two mutations, C25S (below N-terminal Glutamine) and H78Q (above N-terminal Glutamine) shown as dashed lines.

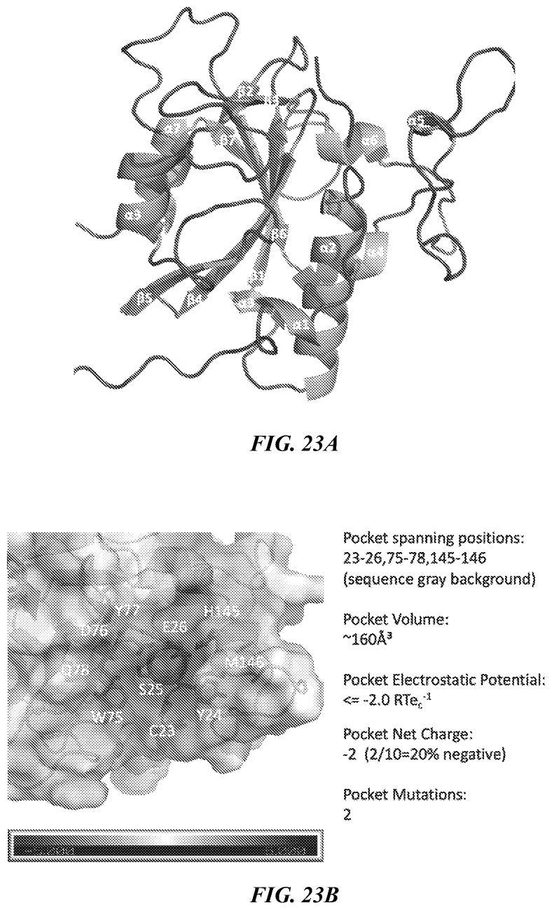

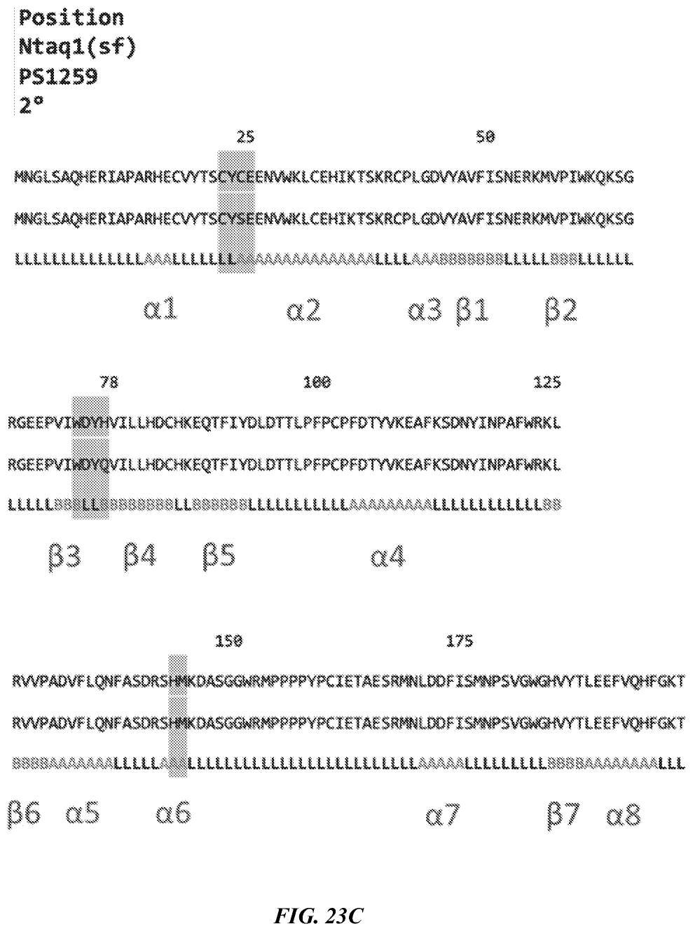

FIGS. 23A-23C show secondary structure, sequence, and binding pocket properties of PS1259. FIG. 23A shows a classification of the protein into secondary structure groups. FIG. 23B shows Poisson Boltzmann electrostatic potential surface map of binding pocket with residues labeled that form the binding pocket, with corresponding pocket properties listed. FIG. 23C shows the sequence of natural parent protein and engineered variant highlighting mutations, pocket positions and secondary structure assignment per position.

Example 15 provides additional experimental details relating to PS1259 and structurally-homologous recognizers for use in protein sequencing.

### Example 9. Direct Identification of Arginine Post-Translational Modifications

Proteins undergo a diverse array of post-translational modifications (PTMs) to their amino acid side chains that can strongly affect protein function and mediate intricate cellular events. Measuring the diversity, dynamics, and functional consequences of PTM states of proteins across the proteome is essential to understanding the role of proteins in health and disease. However, discovery and detection of PTMs and routine measurement of complex PTM states remains highly challenging and the diversity of proteoforms in the human proteome remains largely unmapped. New methods to enable sensitive detection of PTMs will greatly aid biomarker discovery, drug discovery, and the development of precision and personalized approaches to medicine.

Modifications of the arginine side chain are of particular biomedical interest. Methylation and citrullination of arginine residues in a number of human proteins have been shown to play key roles in disease states such as cardiovascular disease, autoimmune disease, and cancer. In this example, aspects of the technology described herein were applied to the detection of arginine methylation and citrullination with single-molecule resolution and sensitivity.

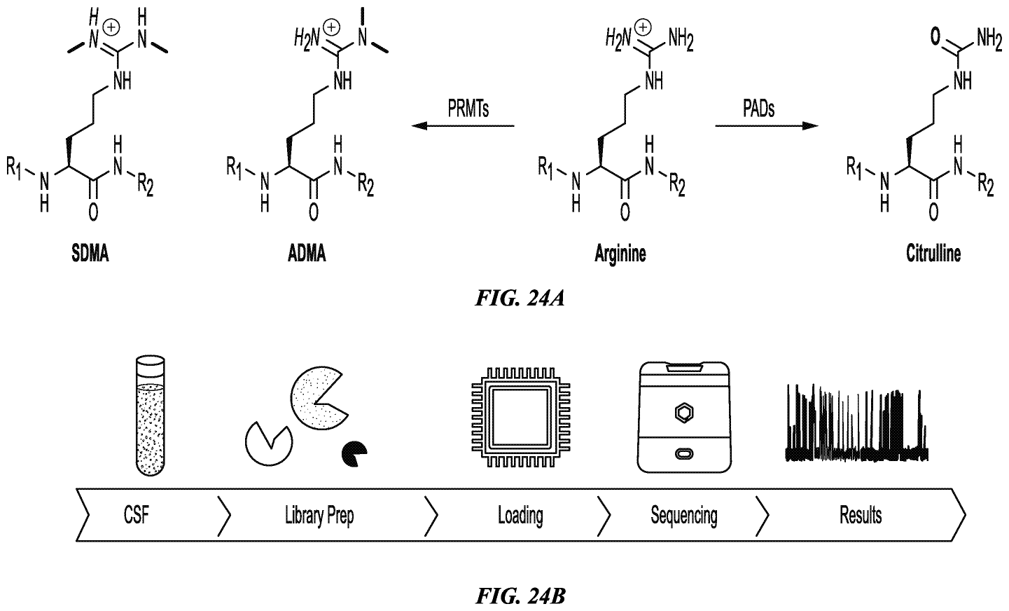

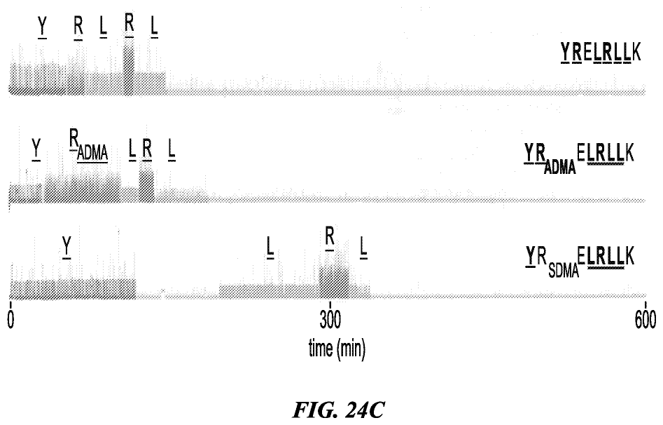

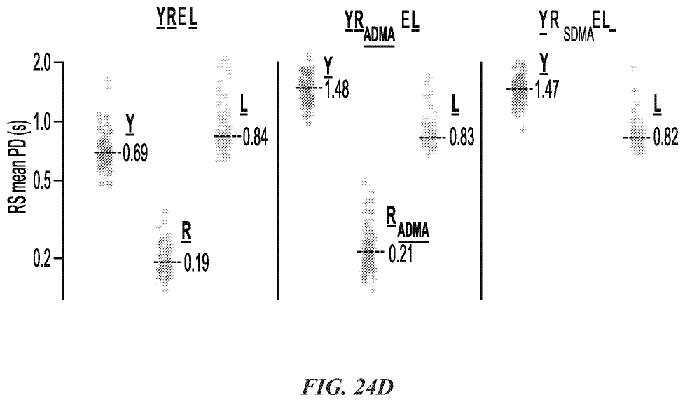

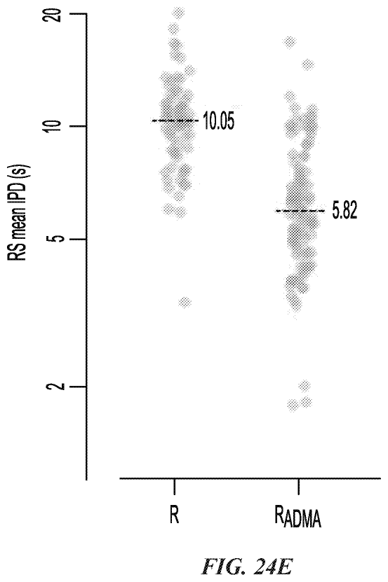

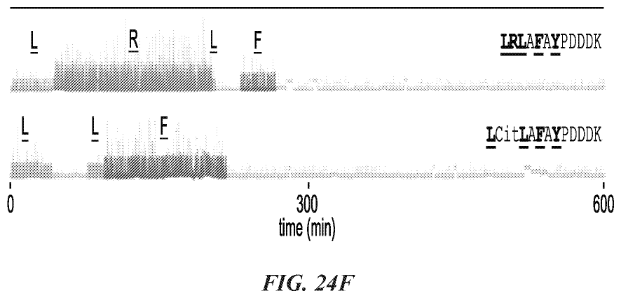

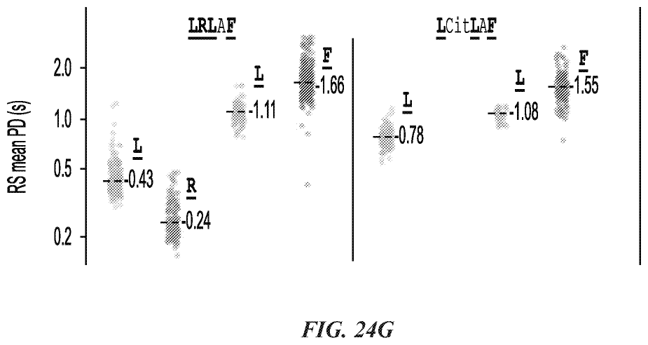

Arginine plays an important role in protein structure and function due to the unique properties of the guanidinium group that forms the terminus of its side chain (FIG. 24A). This group is both positively charged and capable of forming extended hydrogen bond networks and cation-π interactions with other amino acids and with nucleic acids. Arginine, therefore, often mediates key interactions between protein binding partners or between proteins and DNA.

The two most common arginine PTMs, dimethylation and citrullination, alter the arginine side chain and change its properties (FIG. 24A), potentially resulting in important downstream effects on cellular processes. Dimethylation retains arginine's positive charge but increases its size and hydrophobicity and blocks hydrogen bond formation. Citrullination eliminates arginine's positive charge, resulting in a neutral side chain with altered properties that can greatly impact protein conformation and function.

Dimethylation and citrullination of arginine are carried out by enzymes and may be part of the normal regulation of cellular processes or involved in disease states. Arginine dimethylation is catalyzed by protein arginine methyltransferases (PRMTs). PRMTs transfer two methyl groups either asymmetrically onto the same nitrogen atom, resulting in asymmetric dimethyl arginine (ADMA) or symmetrically onto opposite nitrogen atoms, resulting in symmetric dimethyl arginine (SDMA). These modifications increased size and hydrophobicity and block hydrogen bonding. Arginine citrullination is catalyzed by protein arginine deiminases (PADs). PADs carry out the hydrolysis of arginine's positively-charged guanidinium group, resulting in a neutral ureido group. This transformation results in a negligible mass increase of 0.9840 Da, but the loss of positive charge can dramatically alter protein conformation and function. FIG. 24A illustrates the structures of SDMA, ADMA, canonical arginine, and citrulline.

Arginine PTMs have emerged as important targets of biomedical research. Methylated arginine residues and their respective PRMTs have been implicated in important diseases such as cardiovascular disease and cancers. Critical involvement of arginine citrullination in immune system function, skin keratinization, myelination, and the regulation of gene expression has also been demonstrated. Notably, the removal of arginine's positive charge in some cases can cause proteins to activate the immune system, contributing to autoimmune diseases.

### Challenges for the Detection of Arginine PTMs

Research into these arginine PTMs has been particularly challenging because they are difficult to detect and differentiate with current proteomic methods. Mass spectrometry is the most frequently utilized tool for detecting protein PTMs. However, mass spectrometry cannot easily distinguish ADMA and SDMA because they are constitutional isomers with identical mass. Likewise, deamination of arginine to citrulline results in a negligible mass increase of 0.9840 Da. This mass difference can easily be confused with a ¹³C isotope or misinterpreted as deamidation of nearby asparagine or glutamine residues. In addition, mass spectrometry techniques for arginine PTM detection require highly specialized knowledge and training and advanced analysis methods.

Enzyme-linked immunosorbent assay (ELISA), another common method for PTM detection, uses antibodies specifically generated to detect a modified protein of interest. Although arginine PTMs are estimated to be widespread in human cells, commercially available antibodies against arginine PTMs are limited to specific sites on a few highly studied proteins. The requirement to generate new antibodies, along with complex workflows, expense, antibody reproducibility, and other challenges associated with ELISA assay development, is likely to hinder discovery and further study of novel arginine PTM sites.

Continued development toward novel methods is needed to facilitate direct detection of arginine PTMs in proteins. Single-molecule protein sequencing offers an alternative approach to the detection of ADMA, SDMA, and citrulline that is not based on mass to charge ratio or antibody specificity, but rather on the kinetic signature of binding between recognizers and N-terminal amino acids (NAAs).

Aspects of the technology described herein gain insight into these PTMs with single molecule resolution, overcoming current technological gaps, and providing direct detection of arginine PTMs.

### Methodology & Workflow

PTM detection involved isolating peptides and subjecting them to a real-time single-molecule protein sequencing reaction. Proteins were first digested into peptide fragments and conjugated C-terminally to macromolecular linkers. The peptide complexes were immobilized at the bottom of nanoscale wells on a semiconductor chip, resulting in single peptide molecules with exposed N-termini ready for sequencing. During the sequencing reaction, the surface-immobilized peptides were exposed to a solution containing dye-labeled NAA recognizers that bound on and off to their cognate NAAs with characteristic kinetic properties. Aminopeptidases in solution sequentially removed individual NAAs to expose subsequent amino acids for recognition. Fluorescence lifetime, intensity, and kinetic data were collected in real time and analyzed to determine amino acid sequence and PTM content.

The trace-level output included distinct pulsing regions called recognition segments (RSs); each RS corresponded to a period of time between aminopeptidase cleavage events during which an NAA recognizer bound on and off to its exposed target NAA. Chemical modifications to a target NAA or to a nearby downstream amino acid can modulate recognizer affinity, resulting in a characteristic change in the average pulse duration (PD) during an RS relative to an unmodified peptide. These modifications can also influence the rate of aminopeptidase cleavage of an NAA, resulting in a characteristic change in average duration of the corresponding RS.

A summary of the workflow for sequencing of peptides and detection of PTMs is presented in FIG. 24B.

### Results & Discussion

### Detection of Arginine Dimethylation

First, the detection and differentiation of arginine, ADMA, and SDMA by single-molecule protein sequencing was demonstrated. The focus was on a key segment of the signaling protein P38MAPKα. Dimethylation of arginine residue 70 of P38MAPKα in myoblast cells by PRMT7 is a critical regulatory step in the activation of myoblast differentiation in humans.

Synthetic peptides corresponding to residues 69 to 76 of P38MAPKα were generated in three versions containing either arginine, ADMA, or SDMA at position 2: YRELRLLK (SEQ ID NO: 1077), YR_{ADMA}ELRLLK (SEQ ID NO: 1078), and YR_{SDMA}ELRLLK (SEQ ID NO: 1079). Each peptide was sequenced using three recognizers—PS610 (F, Y, W), PS961 (L, I, V), and PS621 (R)—and data were analyzed to identify RSs, determine the mean PD of each RS, and characterize the kinetic signature of each peptide. Each peptide displayed a distinguishable pattern due to the distinct kinetic influences of arginine, ADMA, and SDMA on recognizer binding (see example traces in FIG. 24C).

Arginine and ADMA residues exhibited binding with the recognizer PS621 with similar PD, whereas SDMA exhibited no binding (FIGS. 24C, 24D). This result indicated that symmetric dimethylation of arginine—in contrast to asymmetric dimethylation-reduced the affinity of PS621 for N-terminal arginine, providing a clear kinetic difference between these isomeric arginine PTMs. The NAA recognizers used in this example contact residues at positions 2 and 3 from the N-terminus when they bind to their target NAAs; therefore, modification of these downstream residues can influence recognizer binding affinity. A strong influence of arginine dimethylation on recognition of the upstream tyrosine residue in these peptides by PS610 was observed (FIGS. 24C-24E). The median pulse duration of tyrosine recognition increased from 0.69 s for YRE to 1.47 s and 1.48 s for YRADMAE (SEQ ID NO: 1088) and YRSDMA (SEQ ID NO: 1089), respectively (FIG. 24D). In addition, the median interpulse duration (IPD) of arginine recognition by PS621 decreased from 10.05 s for unmodified arginine to 5.82 s for ADMA (FIG. 24E).

The influence that these dimethylated arginine residues have on the recognition of preceding NAAs serves as a powerful feature of protein sequencing with single-molecule sensitivity and precision. These results demonstrate the capacity for unprecedented sensitivity in detection of arginine dimethylation using aspects of technology described herein.

### Detection of Arginine Citrullination

It was next demonstrated that differential binding kinetics could be used to rapidly differentiate citrullinated arginine residues from native arginine residues. Two synthetic peptide sequences containing either arginine or citrulline at position 2—LRLAFAYPDDDK (SEQ ID NO: 1053) and LCitLAFAYPDDDK (SEQ ID NO: 1080)—were generated and sequenced using three recognizers as described above. Each peptide displayed a highly distinguishable kinetic signature due to the influence of the different arginine and citrulline side chains on recognition (FIGS. 24F-24G). Citrullination eliminated N-terminal arginine recognition by PS621 (see example traces in FIG. 24F). Citrullination at position 2 also resulted in a large increase in the median PD of recognition of the N-terminal leucine located at the preceding position by PS961. Median PD was 0.43 s for LRL increased to 0.78 s for LCitL (FIG. 24G). These results demonstrate the capability to detect and digitally quantify arginine citrullination.

### CONCLUSION

In this example, arginine PTMs were directly detected. Arginine PTMs play important roles in human health and disease but have been challenging to study. Current proteomic methods such as mass spectrometry and ELISA have been capable of just indirect identification of these arginine PTMs using highly specialized techniques or limited to a small set of specific proteins on the basis of antibody availability and other challenges. The ability to directly detect PTMs offers great potential for accelerated biomedical research and for a wide range of commercial applications in drug discovery and biomarker development.

### Example 10. Identification of Threonine Post-Translational Modification

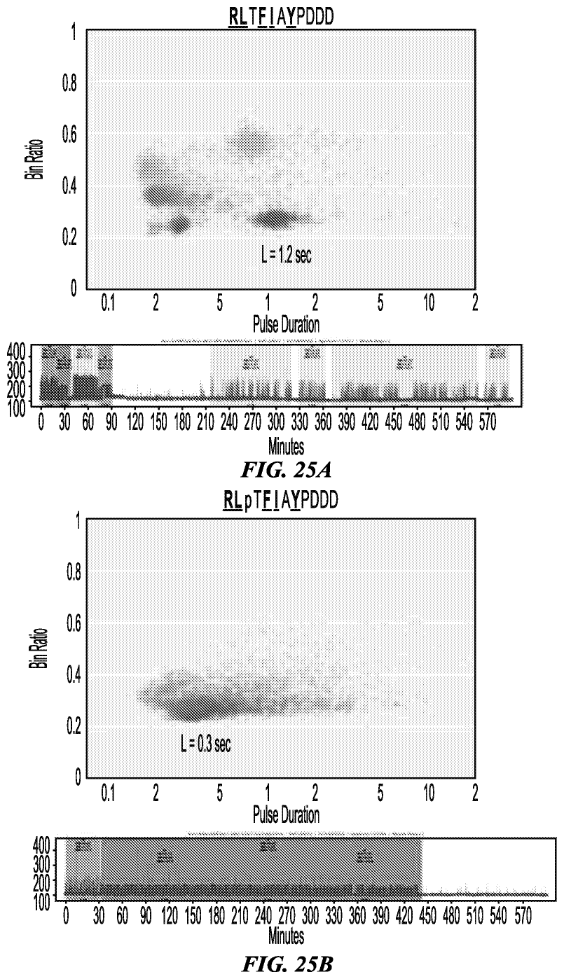

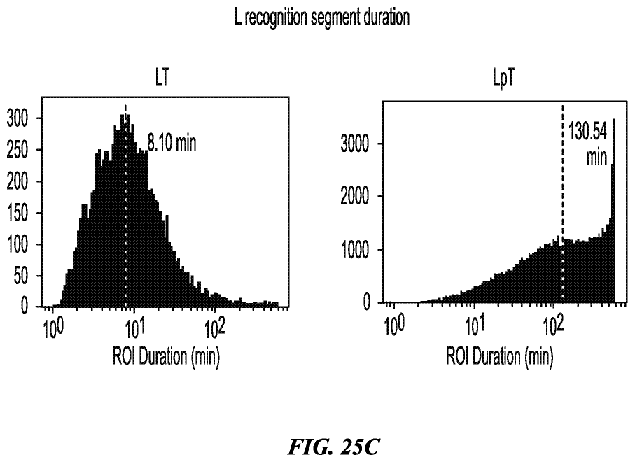

Sequencing reactions using the recognizers PS691 (R), PS610 (FYW), and PS961 (LIV) were performed separately for the peptides RLTFIAYPDDD (SEQ ID NO: 1057) and RLpTFIAYPDDD (SEQ ID NO: 1058) (where pT is phosphothreonine). Recognition of the N-terminal leucine preceding threonine or phosphothreonine by PS961 was observed, with distinct pulse duration for leucine followed by threonine (RS mean PD=1.2 s; FIG. 25A) compared to leucine followed by phosphothreonine (RS mean PD=0.3 see; FIG. 25B). Moreover, recognition segment (RS) durations for leucine recognition were longer when leucine was followed by phosphothreonine (RS mean duration=130 min; FIG. 25C, right panel) compared to threonine (RS mean duration=8.1 min; FIG. 25C, left panel). These data demonstrate the ability to discriminate between unmodified and post-translationally modified threonine side chains.

### Example 11. Identification of Tyrosine Post-Translational Modification

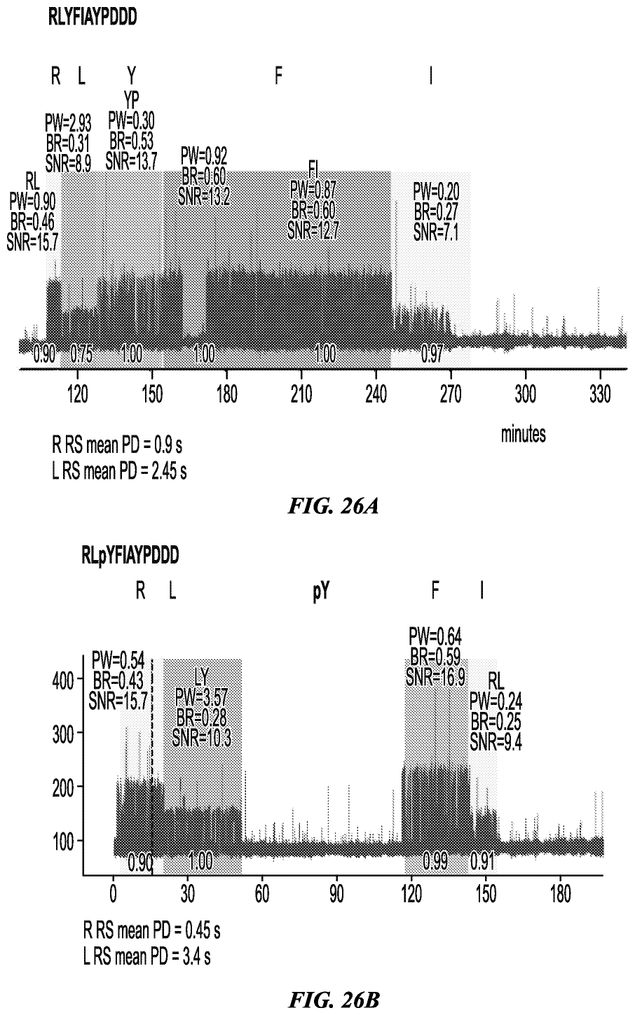

Sequencing reactions using the recognizers PS691 (R), PS610 (FYW), and PS961 (LIV) were performed separately for the peptides RLYFIAYPDDD (SEQ ID NO: 1059) and RLpYFIAYPDDD (SEQ ID NO: 1060) (where pY is phosphotyrosine). Recognition of the N-terminal arginine and leucine residues preceding tyrosine or phosphotyrosine by PS691 and PS961, respectively, was observed, with distinct pulse durations depending on whether the peptide contained tyrosine (FIG. 26A) or phosphotyrosine (FIG. 26B). Recognition of N-terminal arginine occurred with RS mean PD of 0.9 s for RLY and 0.45 s for RLpY. Recognition of N-terminal leucine occurred with RS mean PD of 2.45 s for LYF and 3.4 s for LpYF. Moreover, traces from the peptide RLpYFIAYPDD (SEQ ID NO: 1081) contained a consensus gap between L and F, since pY was not recognized by PS610, whereas traces from the peptide RLYFIAYPDDD (SEQ ID NO: 1059) contained Y recognition by PS610 during this interval. These data demonstrate the ability to discriminate between unmodified and post-translationally modified tyrosine side chains.

### Example 12. Identification of Lysine Post-Translational Modification

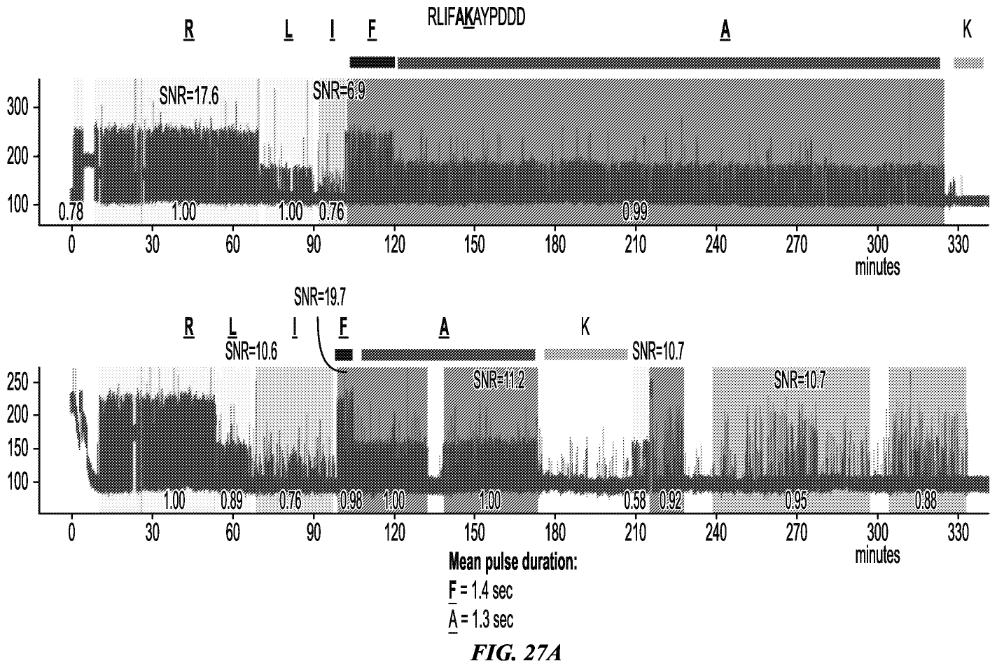

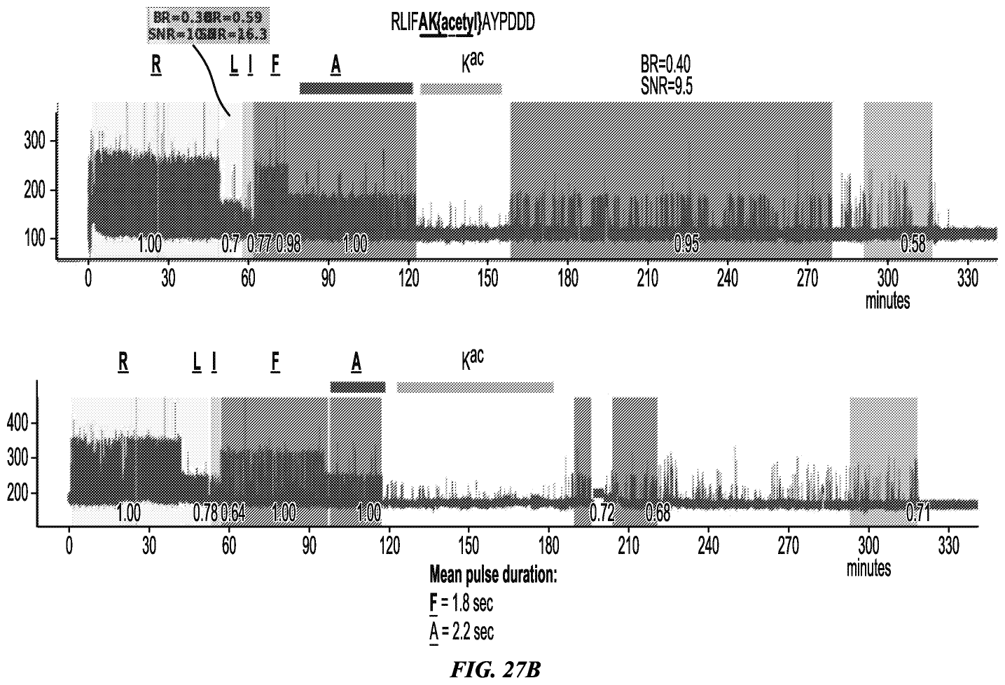

Sequencing reactions using the recognizers PS691 (R), PS610 (FYW), PS961 (LIV), and PS1165 (A) were performed separately for the peptides RLYFKAYPDDD (SEQ ID NO: 1061) and RLK{acetyl}FIAYPDDD (SEQ ID NO: 1062) (where K{acetyl} is a acetylated lysine). Recognition of the N-terminal phenylalanine and alanine residues preceding lysine or acetyl-lysine by PS610 and PS1165, respectively, was observed, with distinct pulse durations depending on whether the peptide contained lysine (F=1.4 s, A=1.3 s; FIG. 27A) or acetylated lysine (F=1.8 s, A=2.2 s; FIG. 27B). Recognition of N-terminal phenylalanine occurred with RS mean PD of 1.4 s for FAK and 1.8 s for FAK{acetyl}. Recognition of N-terminal alanine occurred with RS mean PD of 1.3 s for AK and 2.2 s for AK{acetyl}. These data demonstrate the ability to discriminate between unmodified and post-translationally modified lysine side chains.

### Example 13. Identification of Beta-Amyloid Variants Introduction and Significance

Alzheimer's is a neurogenerative disease that affects tens of millions of people worldwide and carries no clear genetic marker. A hallmark of Alzheimer's is the accumulation of mutated beta-amyloid proteins, creating plaques around neurons that disrupt normal cell function in the brain. The technology described herein may be used to sequence and identify key β-amyloid variants that are indicative of early-onset Alzheimer's, which enables understanding of the underlying disease's pathway to further optimize treatment responsiveness and identify targets with therapeutic potential.

Alzheimer's is a very complex disease and less than 1% of cases can be connected to a single inherited gene. Therefore, DNA sequencing alone can only give a limited view of the disease, its causes, and its pathways; further exploration of the disease mechanisms must occur at the protein level. There is evidence that point mutations in β-amyloid can lead to protein misfolding, which can contribute to the cause of disease or provide markers for early disease progression. Several variants of β-amyloid have been shown to induce misfolding, which exposes hydrophobic regions and causes protein deposition around neurons, then altering cellular function in the brain. The fibril forming peptides 16KLVF19 (SEQ ID NO: 1082) and 17 LVFF20 (SEQ ID NO: 1083) have been explored for targeted drug developments via the β-sheet breaker mechanism.

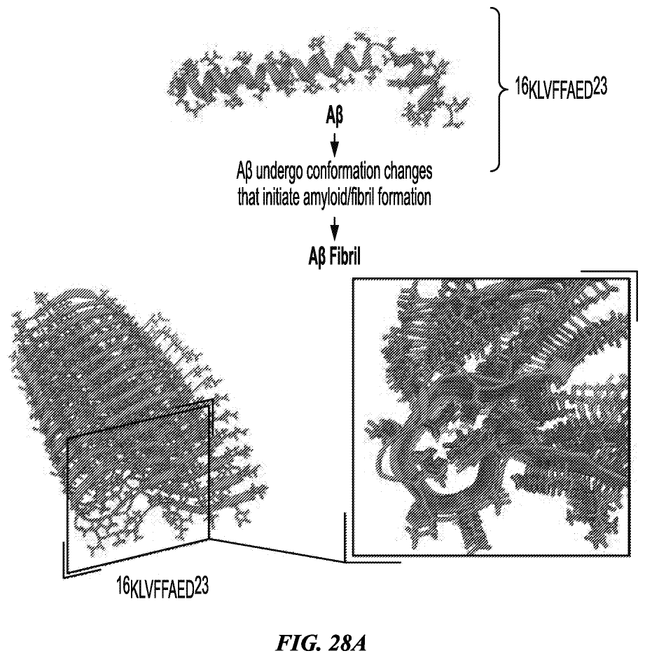

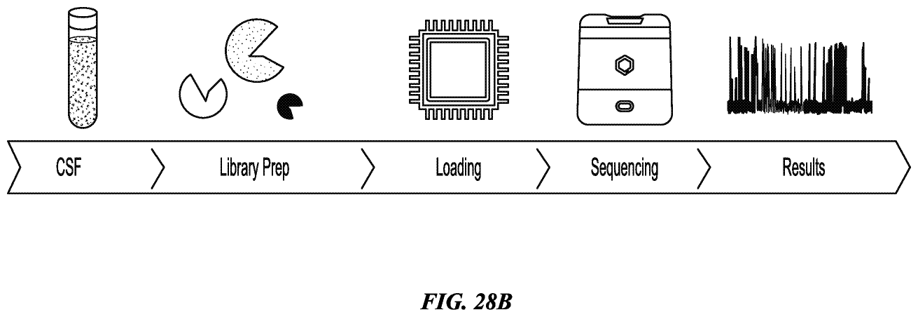

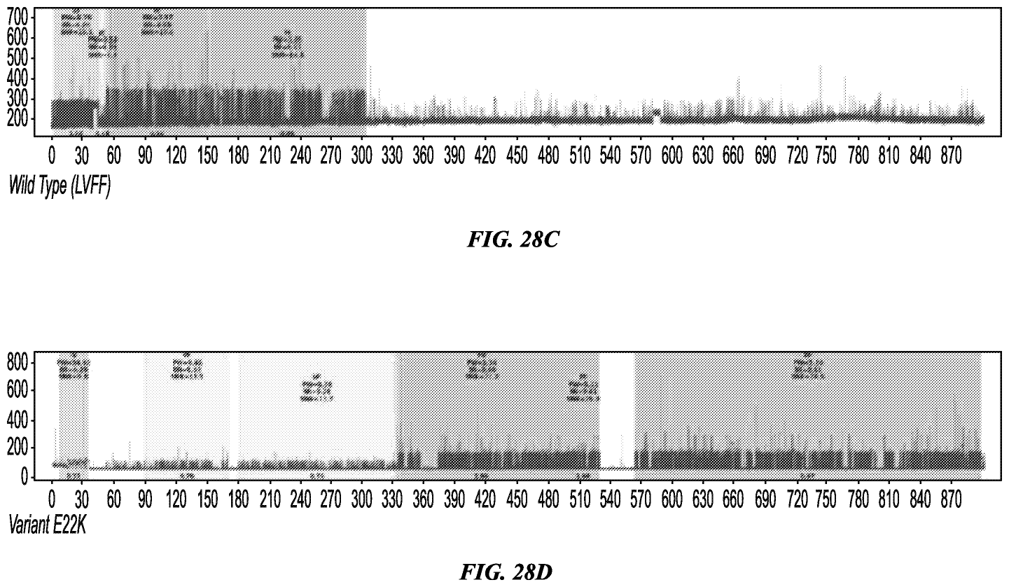

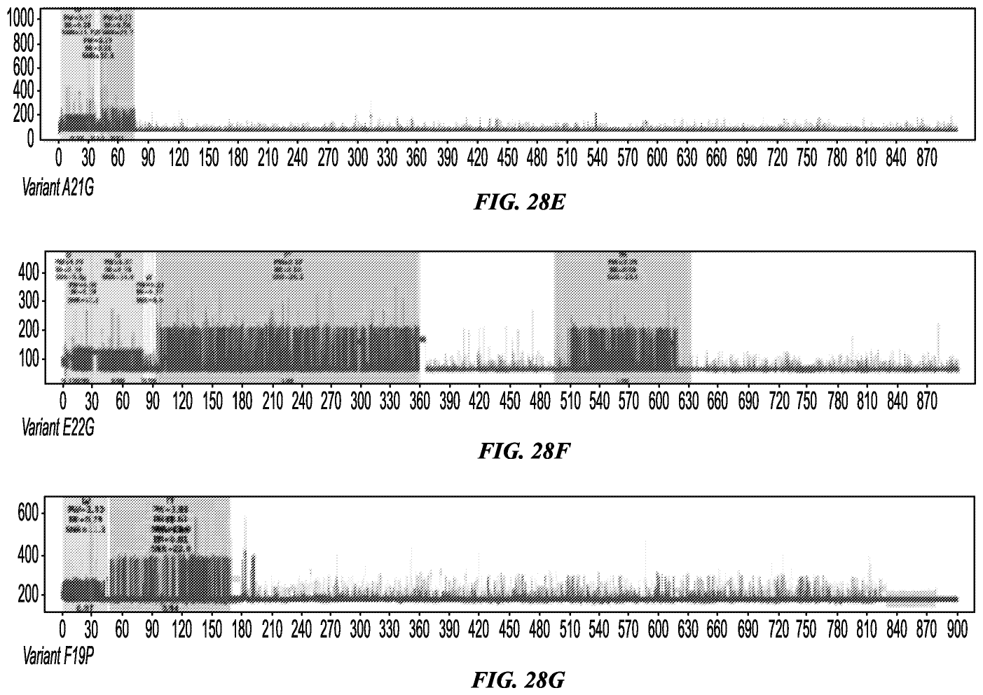

FIG. 28A illustrates an example of a β-amyloid variant. The β-amyloid variant induces misfolding of the protein, exposing hydrophobic regions, which induces aggregation. This alteration in structure morphs the β-amyloid into long filamentous chains or fibril formation, which generate insoluble deposits, which are referred to as pathological plaque.

The research around different types of recognizable proteins and potential PTMs has been largely limited in traditional proteomics. β-amyloid plaque formation is shown to be driven by a single mutation in a folded region of the protein, making their presence challenging to detect by legacy proteomic methods. Aspects of the technology described herein may be used to assess proteins at the individual amino acid level without the need for developing binding affinity assays, an invaluable tool to fully understand biological processes and monitor disease states directly.

### Methodology and Workflow

Aspects of the technology described herein may be used for protein preparation, peptide library preparation, peptide sequencing and peptide profiling of synthetic samples of β-amyloid. In this example, the wild type (LVFFAE (SEQ ID NO: 1063)) and variants (17 LVFFAK22 (SEQ ID NO: 1064), 17 LVFFGK22 (SEQ ID NO: 1065), 17 LVFFAG22 (SEQ ID NO: 1066), and 17 LVPFAE22 (SEQ ID NO: 1067)) of β-amyloid were digested and labeled for further analysis. Alternatively, β-amyloid may be purified from common sources, such as cerebrospinal fluid (CSF), for downstream analysis.

FIG. 28B illustrates an example workflow for β-amyloid variant detection. As shown in FIG. 28B, β-amyloid cerebrospinal fluid may be isolated. A peptide library may be prepared utilizing specific proteolytic enzymes. The sample may be loaded onto a chip, as described herein, and sequencing may be run. Results may include identification of amino acid sequences that demarcate potential Alzheimer's causing mutations.

A chip including aspects of the technology described herein was used for the downstream sequencing of the sample material. The chip contained millions of wells, each of which acted as an independent sequencing machine. Once the sample was loaded, cloud technology was used to set up the sequencing run and collect all the data for visualization. Once collected in the cloud, a set of proprietary algorithms, which can identify amino acids based on the specialized optical pulse patterns of each binding event, determined the sequence of the peptide and mapped that sequence back to a specific protein or protein variant.

Here, the protein sequencing technology and analysis pipeline was successfully used to distinguish a variety of clinically significant β-amyloid point mutations. The sequencing traces containing pulse patterns of the variants, 17 LVFFAK22 (SEQ ID NO: 1064), 17 LVFFGK22 (SEQ ID NO: 1065), 17 LVFFAG22 (SEQ ID NO: 1066), and 17 LVPFAE22 (SEQ ID NO: 1067), were compared to the wild type, 17 LVFFAE22 (SEQ ID NO: 1063). These patterns are shown in FIGS. 28C-28G. Software automatically identified pulses containing the same intensity, lifetime, and kinetics, which determined a recognition segment for a specific amino acid. Each recognition segment was color coded based on the N-terminal amino acid, and the collection of recognition segments provided a characteristic signature of the peptide.

Time domain sequencing functionality can observe sequence changes indirectly during peptide profiling. The specific PTMs and folding of each variant cause them to display distinctly different patterns, which can then be inferred via alterations in pulse width. For example, a point mutation in a sequence—at the N-terminal end, or at the penultimate and antepenultimate positions, of the peptide—can generate an altered pulse pattern, compared to another sequence.

TABLE 14
Average Pulse Width of Tripeptides and Variants

| Tripeptide | Variant | Average Pulse Width |
|---|---|---|
| LVX | WT | 3.26 (LVF) |
| | F19P | 1.86 (LVP) |
| FXX | WT | 2.43 (FAE) |
| | E22G | 2.99 (FAG) |
| | E22K | 2.79 (FAK) |
| | E22Q | 2.67 (FAQ) |
| | A21G | 1.31 (FGE) |

This was shown with the wild type tripeptide LVF and the tripeptide LVP from the F19P mutant. A mutation in the antepenultimate position changed the average pulse width when sequencing L from 3.26 seconds to 1.86 seconds. Likewise, the pulse width for the FAE tripeptide in the wild type changed from an average pulse width of 2.43 seconds to between 1.31 and 2.99 seconds for the mutants. Each change in pulse width provided a hint of change, and each amino acid was potentially interrogated three times when it was at the antepenultimate, penultimate, and N-terminal position. Integration of each piece of evidence can further improve the detection of mutations and PTMs.

This example demonstrates the ability to leverage aspects of the technology described herein to detect single amino acid changes known to be linked to disease progression and severity in β-amyloid. The ease of use and benchtop form factor make the technology described herein available to any lab to leverage in the analysis of other protein families to address a range of important questions related to cell and tissue function in regular and disease scenarios.

### Example 14. Computational Modeling

The modeling of substrates was done on the crystal structure of the Arginine binder. The Arginine binder structure was processed using the Protein Preparation Wizard in Schrodinger suite v. 2022-2. Peptide ligands preparation was performed using LigPrep. A 20 Å cubic box having Asp78 at its center was defined for the docking runs.

Default settings were used under the SP-Peptide mode to dock the prepared peptide ligands using OPLS-2005 force field in the Glide docking toolkit available in Schrodinger suite. Post-docking minimization was performed for all poses. The binder-peptide poses with the best Glide docking scores were subjected to 1 μs molecular dynamics simulations using Desmond. Simulations were carried out using OPLS4 force field in explicit solvent with the SPC water model. Cl⁻ and Na⁺ counterions were added at 0.15 M concentration to keep the system neutral. The binding energy calculation was performed using MM-GBSA methodology (generalized Born and surface area solvation), using 50 evenly spaced snapshots from the entire MD run.

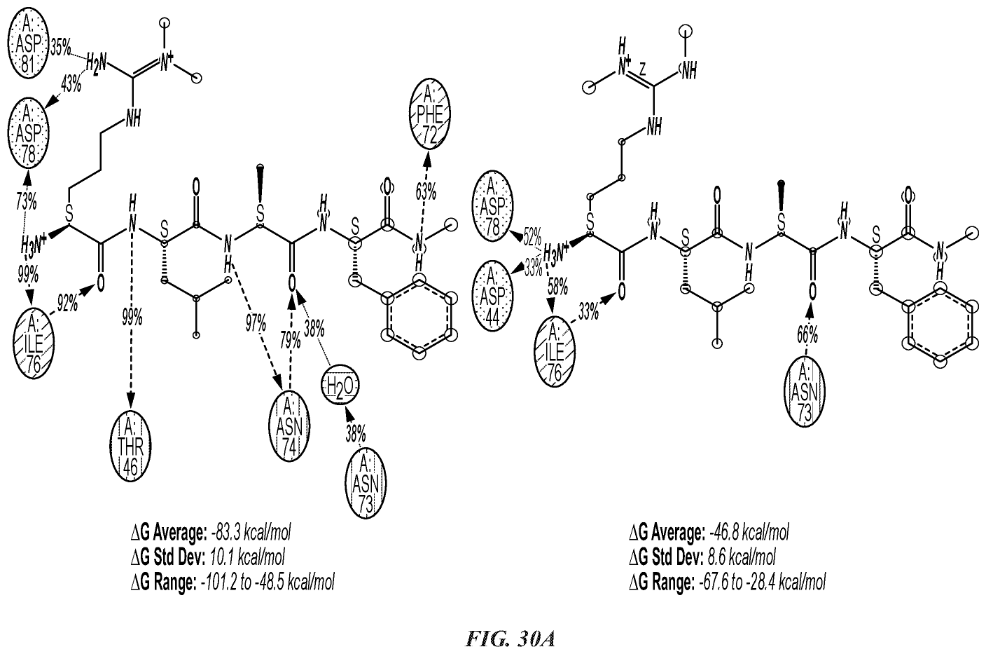

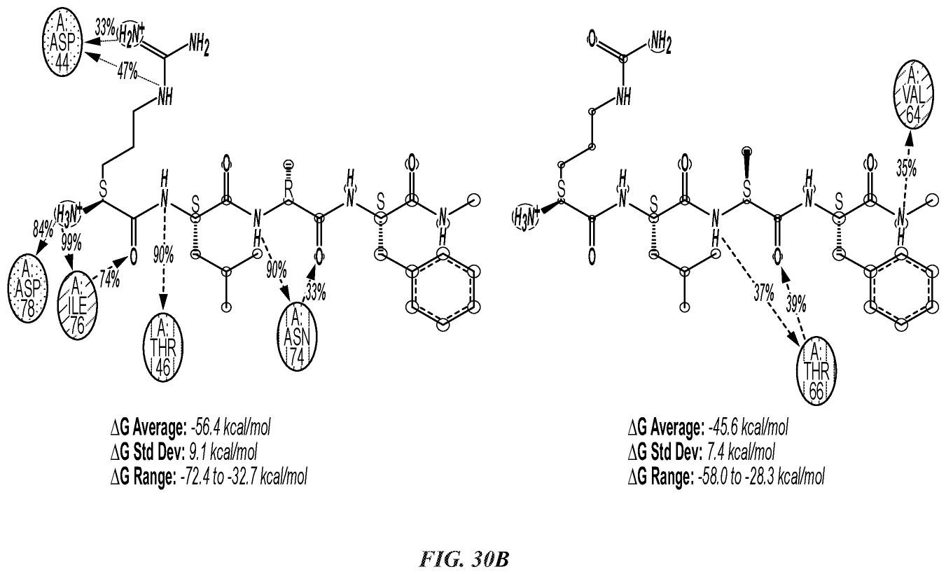

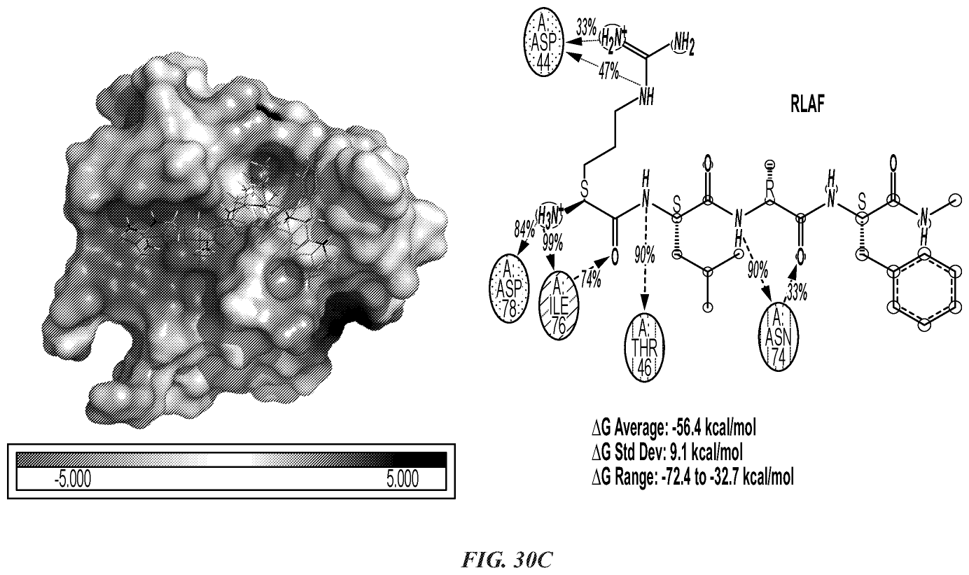

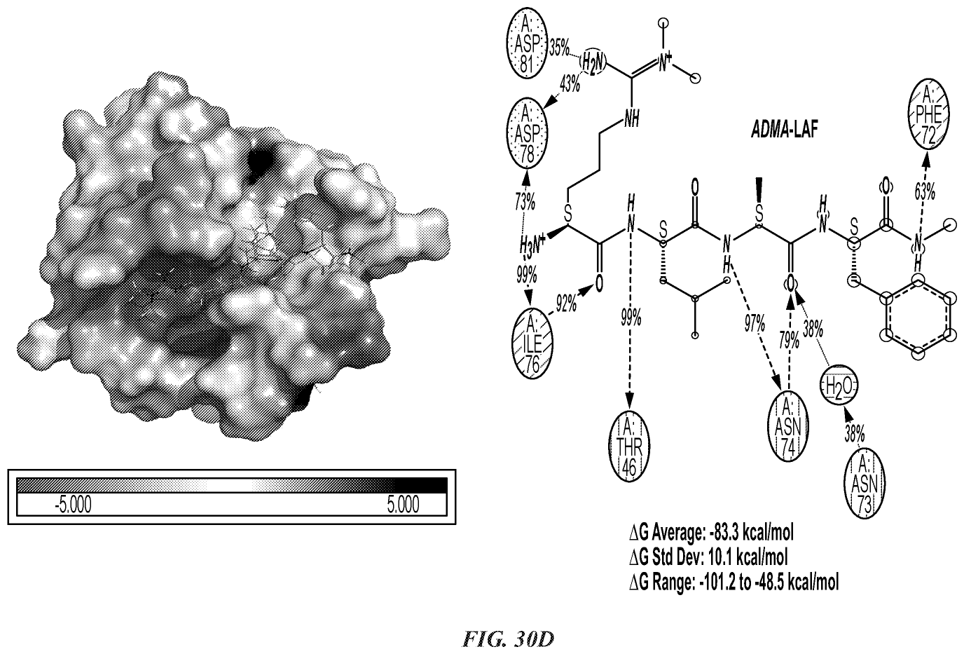

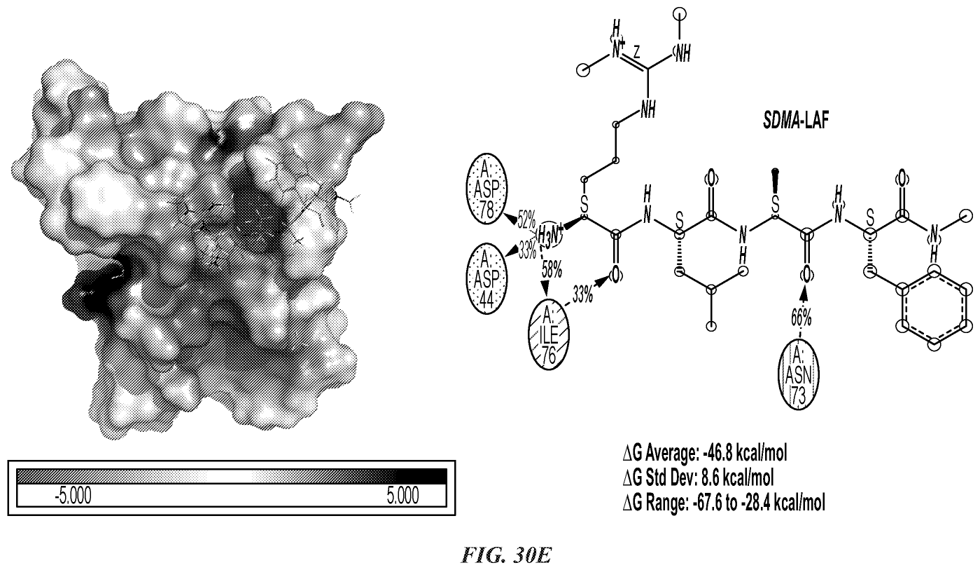

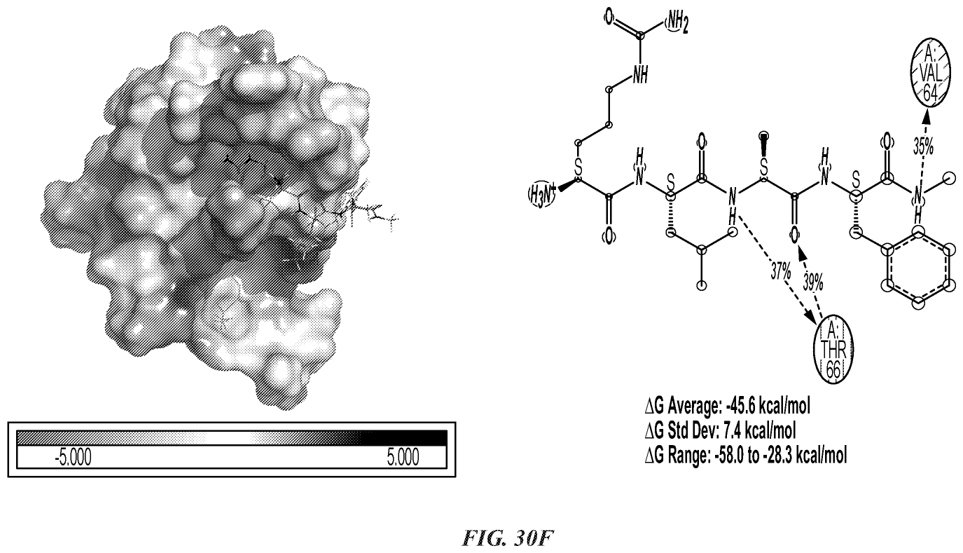

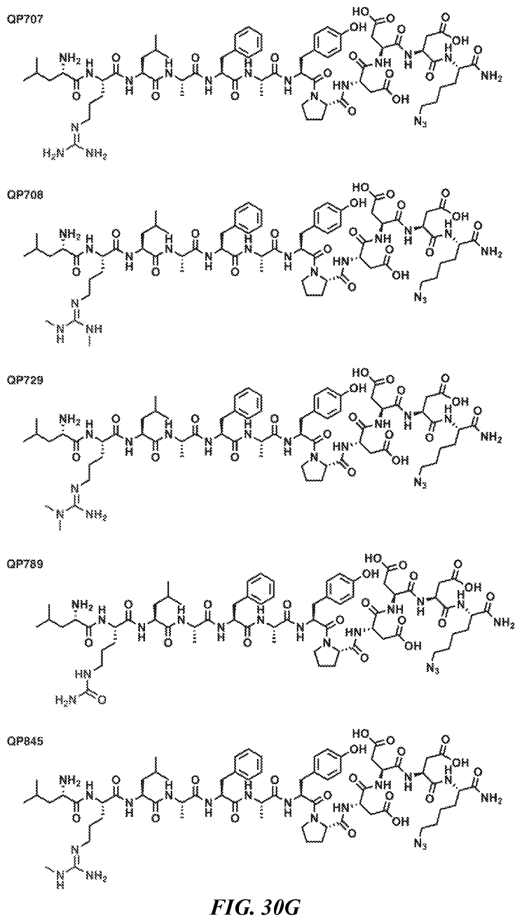

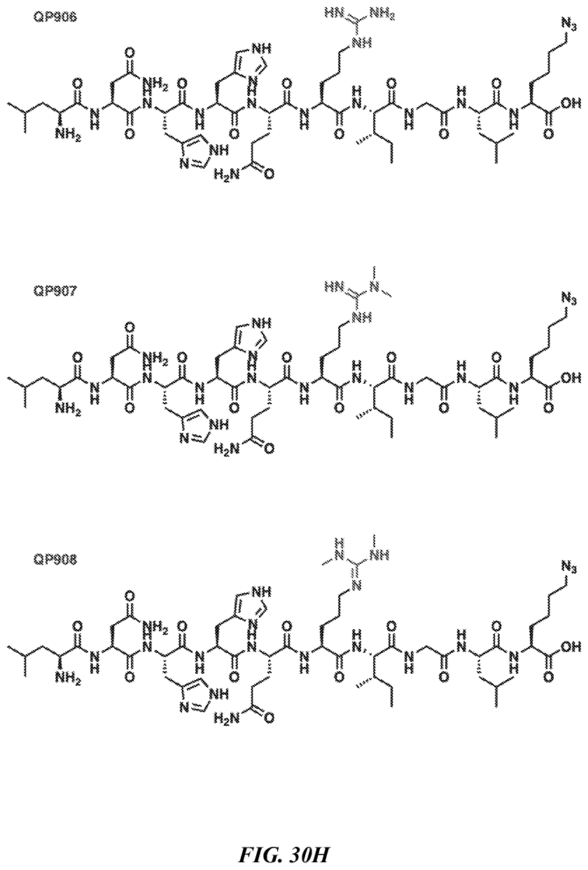

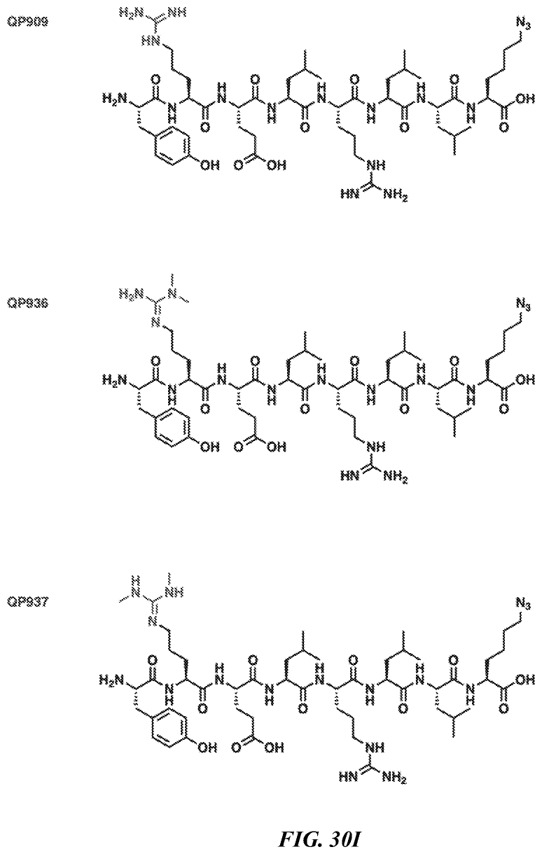

The results from computational modeling of model peptides bound to PS621 are shown in FIGS. 30A-30F. FIGS. 30A-30B show a binding energy comparison for the model peptides QP729/ADMA-LAF (SEQ ID NO: 1143) (top left), QP708/SDMA-LAF (SEQ ID NO: 1144) (top right), QP707/RLAF (SEQ ID NO: 1142) (bottom left), and QP789/Cit-LAF (SEQ ID NO: 1145). FIG. 30C depicts PS621 bound to QP707/RLAF (SEQ ID NO: 1142). FIG. 30D depicts PS621 bound to QP729/ADMA-LAF (SEQ ID NO: 1143). FIG. 30E depicts PS621 bound to QP708/SDMA-LAF (SEQ ID NO: 1144). FIG. 30F depicts PS621 bound to QP789/Cit-LAF.

Structures of model peptides evaluated experimentally and computationally with PS621 and PS1122 are shown in FIGS. 30G-30I.

### Example 15. Development of Ntaq1-Homologous Recognizers

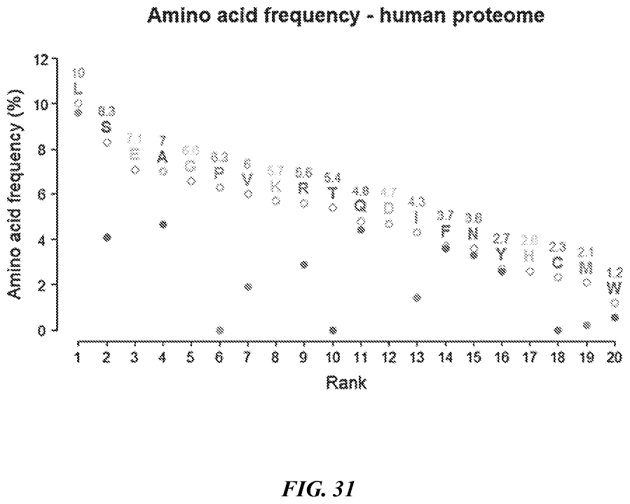

This example describes the development of variants of a Glutaminase (Scleropages formosus) with engineered deactivation of catalysis and peptide binding enhancements, including PS1259 and the structurally homologous variant PS2132. As detailed in Example 8, PS1259 is an engineered variant with improved binding properties for recognizing glutamine and asparagine, and this was attributed in part to a mutation in the catalytic triad (H78Q). Through directed evolution, protein engineering, and subsequent evaluation, it was discovered that an alternative mutation at the same position (H78K) changed the homolog from an improved glutamine/asparagine recognizer to a glutamate recognizer in PS1875, which led to development of PS2132 via several rounds of directed evolution and protein engineering guided by protein ensemble and single molecule kinetic analysis. As shown in FIG. 31, glutamate is among the most frequently occurring amino acids in the human proteome, comprising more than 7% of the human proteome. The ability to detect glutamate using glutamate recognizers, therefore, offers a large and beneficial expansion in proteome coverage in protein sequencing.

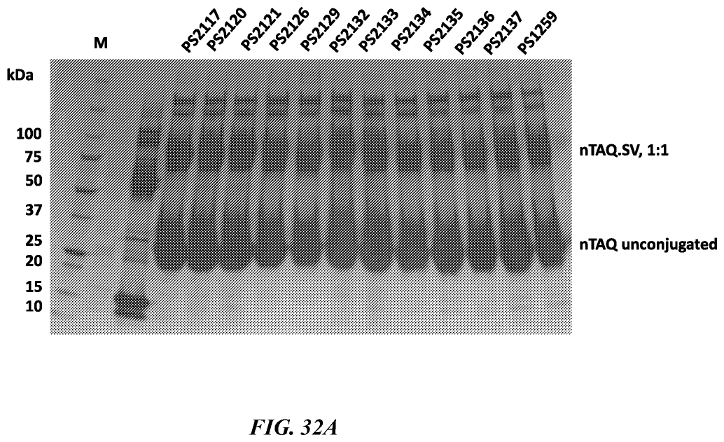

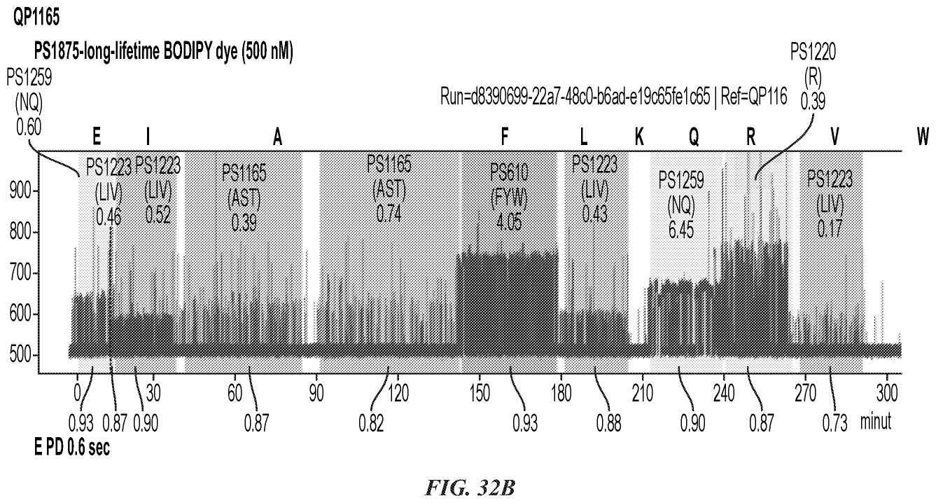

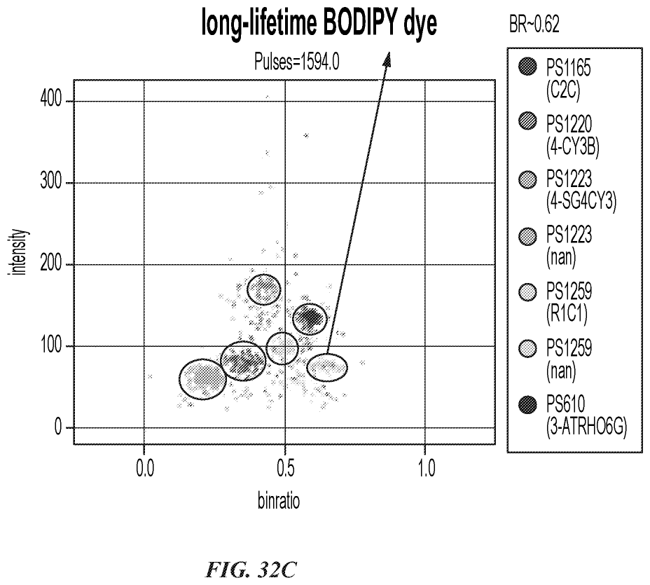

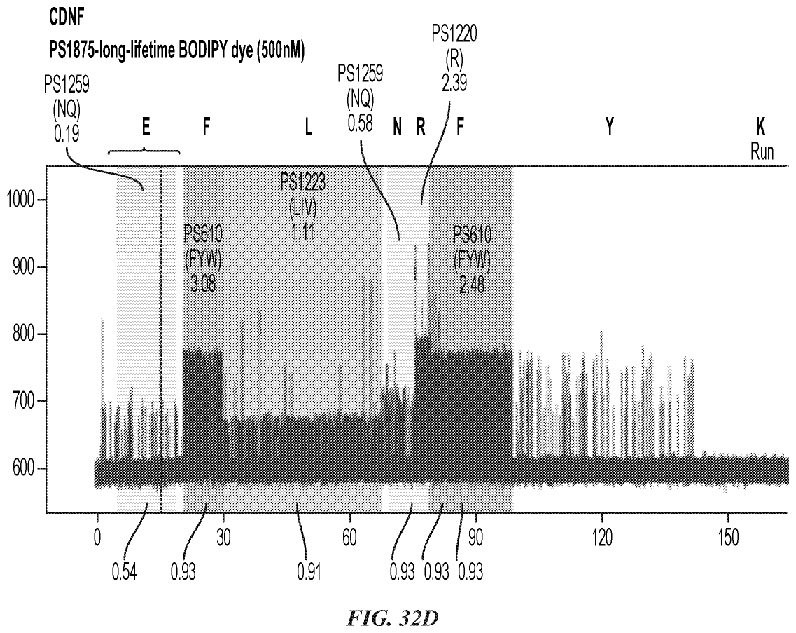

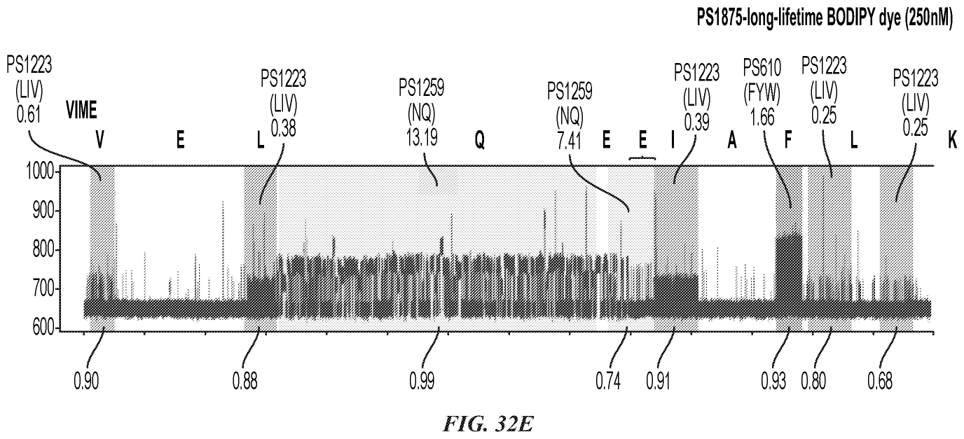

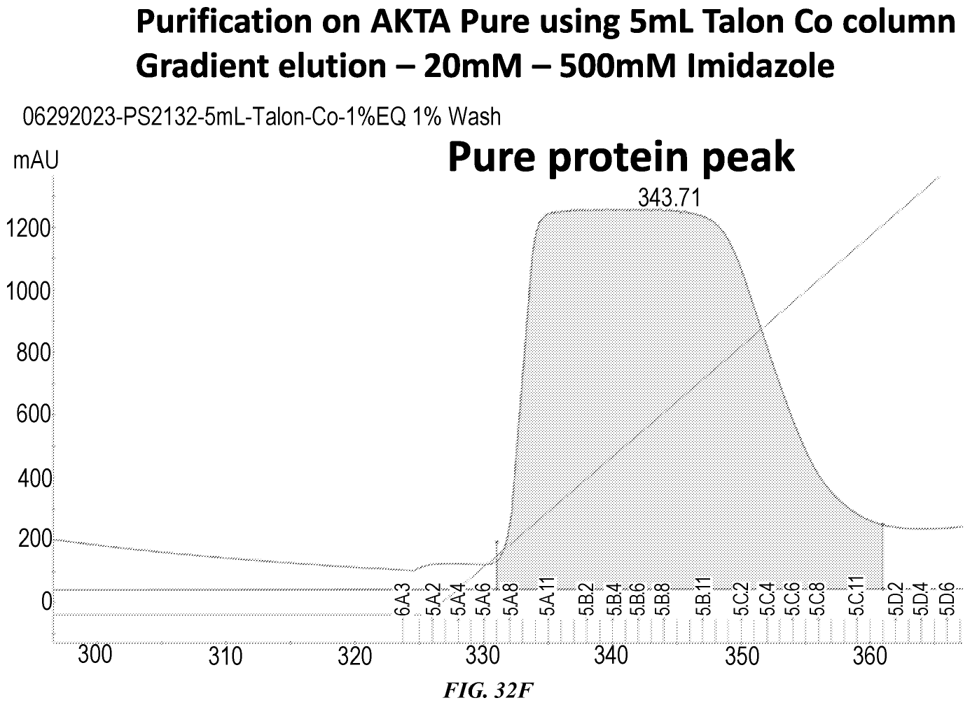

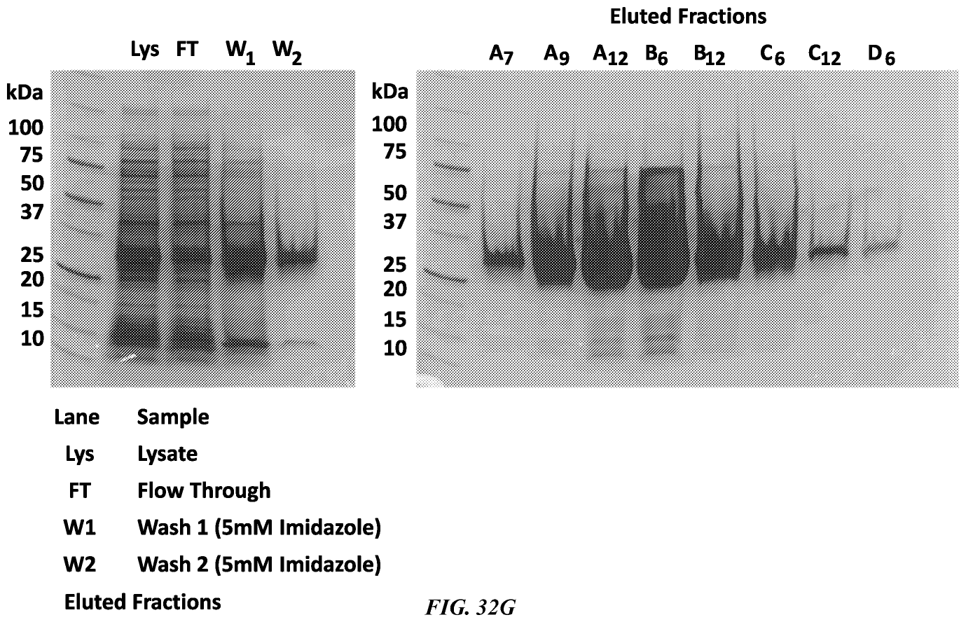

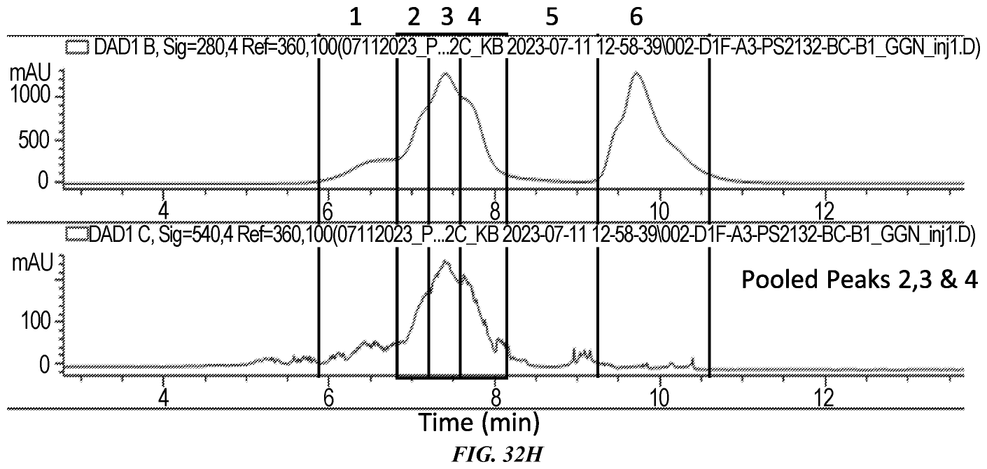

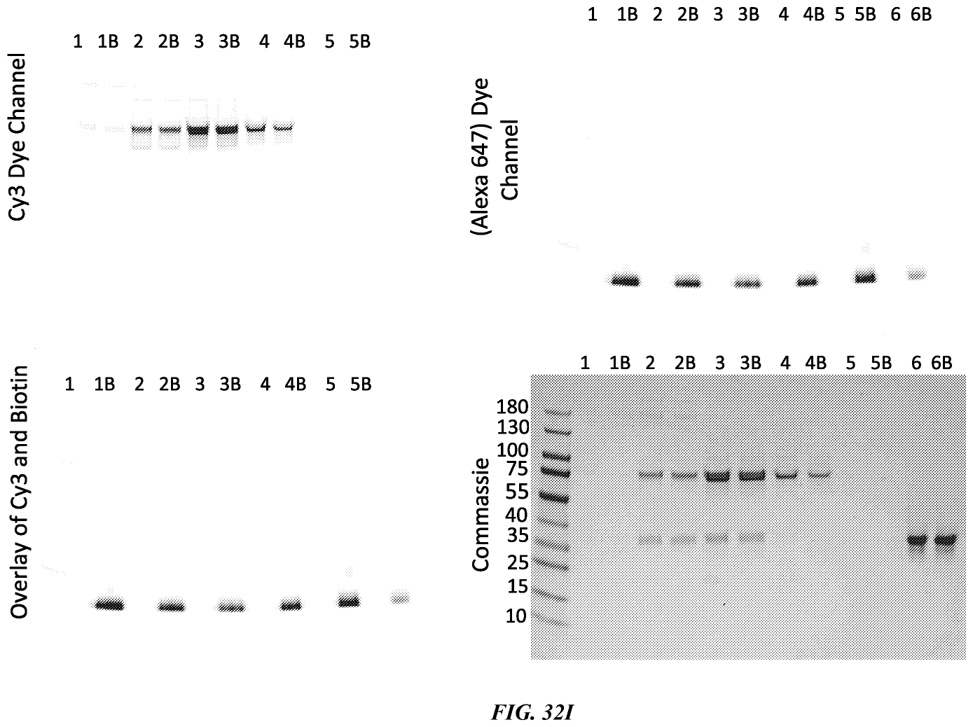

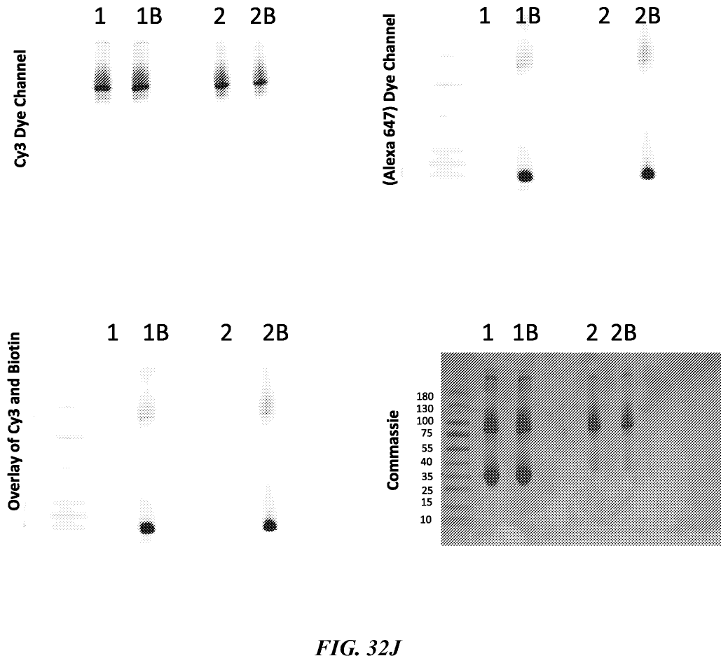

Ntaq1-homologous protein candidate recognizers were identified by directed evolution, expressed in E. coli and purified (FIG. 32A). The candidates were evaluated for binding to N-terminal amino-acids on the Octet platform. The peptides used in the assay contained a penultimate alanine and an N-terminal asparagine (NA), glutamine (QA), glutamate (EA), or aspartate (DA). The set of Octet response measurements is summarized in Table 15 (an empty cell indicates not measured or candidate did not express protein). These results led to the identification of PS1259 (Q and N recognizer) and PS2132 (E recognizer).

TABLE 15
Octet response for Ntaq1-homologous variants

| Binders | NA | QA | EA | DA | Homologs/Mutations |
|---|---|---|---|---|---|
| PS1246 | 0.1 | 0 | 0 | 0.01 | hntaq1 |
| PS1247 | 0.1 | 3 | 0 | 0.01 | hntaq1 + C28S |
| PS1248 | 1.7 | 3.4 | 0.1 | 0.01 | hntaq1 + C28S, H81Q |
| PS1249 | 0 | 0.1 | 0 | 0 | hntaq1 + C28S, H81Q, M149E |
| PS1250 | 0.8 | 3.3 | 0.6 | 0.62 | hntaq1 + C27S |
| PS1251 | 1.7 | 2.5 | 0 | 0.03 | hntaq1 + C27S, H80Q |
| PS1252 | 1.6 | 1.8 | 0 | 0.04 | hntaq1 + C27S, H80Q, M148E |
| PS1253 | | | | | hntaq1 + C27S |
| PS1254 | | | | | hntaq1 + C27S, M147E |
| PS1255 | 0.7 | 3 | 0.9 | 1.02 | hntaq1 + C22S |
| PS1256 | 1 | 2 | 0.6 | 1.18 | hntaq1 + C22S, H75Q |
| PS1257 | 0 | 0 | 0 | 0.1 | hntaq1 + C22S, H75Q, M143E |
| PS1258 | 0 | 3.4 | 0 | 0.02 | hntaq1 + C25S |
| PS1259 | 1.7 | 3.6 | 0.1 | 0.03 | hntaq1 + C25S, H78Q |
| PS1260 | 0.1 | 0.1 | 0 | 0.03 | hntaq1 + C25S, H78Q, M146E |
| PS1261 | 0.1 | 3.4 | 0 | 0.04 | hntaq1 + C25S |
| PS1262 | 0.1 | 1.3 | 0 | 0.01 | hntaq1 + C25S, H78Q |
| PS1263 | 0 | 0 | 0 | 0.01 | hntaq1 + C25S, H78Q, M146E |
| PS1264 | 0 | 2.8 | 0 | 0.01 | hntaq1 + C25S |
| PS1265 | 0 | 0 | 0 | 0.02 | hntaq1 + C25S, H78Q |
| PS1266 | 0 | 0 | 0 | 0.04 | hntaq1 + C25S, H78Q, M146E |
| PS1457 | | | | | PS1258 + V29I, Q64L, H78N, M146N |
| PS1458 | | | | | PS1258 + Q64K, P72L, V73L, H78N, L81I, M146Q |
| PS1459 | | | | | PS1258 + S25A, L32V, Q64R, P72F, V73I, H78Q, L81F, |
| | | | | | M146N |
| PS1460 | | | | | PS1258 + S25A, V29A, L32F, Q64K, H78Q, L81V, |
| | | | | | C103Y, M146N |
| PS1461 | 0.0842 | 0.0638 | 0.0562 | | PS1258 + S25A, V29A, L32M, R41K, Q64K, R68Q, |
| | | | | | P72F, V73L, H78N |
| PS1462 | | | | | PS1258 + S25A, V29L, W30F, L32I, Q64P, P72V, V73I, |
| | | | | | H78N, L81V, M146N |
| PS1463 | | | | | PS1258 + S25A, V29L, W30F, L32I, Q64P, P72V, V73I, |
| | | | | | H78N, L81V, P121H, M146N |
| PS1464 | 0.0582 | 0.0592 | 0.0517 | | PS1258 + Q64L, R68Q, H78Q, P121H, M146N |
| PS1465 | 0.4948 | 0.5755 | 0.4586 | | PS1258 + Q64L, R68Q, H78Q, P121H, M146Q |
| PS1466 | | | | | PS1258 + S25A, V29A, L32F, Q64K, H78Q, L81V, |
| | | | | | C103Y, M146Q |
| PS1467 | 0.0943 | 0.215 | 0.0872 | | PS1258 + V29A, W30S, L81I |
| PS1468 | | | | | PS1258 + S25A, V29L, W30F, L32I, Q64P, P72V, V73I, |
| | | | | | H78N, L81V |
| PS1469 | | | | | PS1258 + L32A, Q64K, L81V, M146N |
| PS1470 | | | | | PS1258 + S25A, V29A, L32F, Q64P, P72V, V73I, H78N, |
| | | | | | L81V, M146Q |
| PS1471 | | | | | PS1258 + S25A, V29L, W30F, L32I, Q64L, R68Q, |
| | | | | | H78Q, P121H, M146N |
| PS1472 | 0.1525 | 0.1497 | 0.1698 | | PS1258 + Q64L, R68Q, H78Q, T90P, P121H, M146N |
| PS1473 | | | | | PS1258 + S25A, V29L, L32A, Q64K, P72F, V73I, |
| | | | | | H78N, L81V |
| PS1474 | | | | | PS1258 + S25A, V29L, W30F, L32I, Q64P, P72V, V73I, |
| | | | | | H78N, L81V, 192N, M146Q |
| PS1475 | | | | | PS1258 + S25A, V29A, L32F, Q64K, H78Q, L81V, |
| | | | | | T90P, C103Y, M146N |
| PS1476 | 0.045 | 0.0364 | 0.0571 | | PS1258 + S25A, V29L, W30F, L32I, Q64P, P72V, V73I, |
| | | | | | H78N, L81V, T90P, M146Q |
| PS1477 | | | | | PS1258 + S25A, V29A, L32F, Q64L, R68Q, H78Q, |
| | | | | | P121H, M146N |
| PS1478 | 0.0381 | 0.0215 | 0.0189 | | PS1258 + S25A, V29A, L32F, Q64R, H78Q, L81V, |
| | | | | | M146Q |
| PS1480 | 0.2 | 0.4 | 0.3 | | PS1258 + S25A, V29L, W30F, L32I, Q64P, P72V, V73I, |
| | | | | | H78N, L81V, M146Q |
| PS1481 | 0.6 | 0.8 | 0.5 | | PS1258 + Q64R, H78Q, M146N |
| PS1482 | 0.3 | 0.3 | 0.3 | | PS1258 + Q64K, P72L, V73L, H78N, L81I, M146N |
| PS1483 | 0.2 | 0.3 | 0.3 | | PS1258 + S25A, W30F, L32A, R41S, G69S, P72F, V73I, |
| | | | | | H78Q, L81I, M146Q |
| PS1484 | 0.3 | 0.2 | 0.3 | | PS1258 + S25A, V29L, Q64P, P72L, V73W, H78Q, |
| | | | | | L81I, M146N |
| PS1485 | 0.4 | 0.3 | 0.4 | | PS1258 + Q64P, P72V, V73I, H78N, L81V, M146Q |
| PS1486 | 0.7 | 0.6 | 0.3 | | PS1258 + Q64L, R68Q, H78Q, M146N |
| PS1487 | 0.7 | 0.6 | 0.5 | | PS1258 + Q64L, R68Q, H78Q, M146Q |
| PS1488 | 0.3 | 0.3 | 0.3 | | PS1258 + E26K, V29L, Q64K, P72L, V73W, H78N, |
| | | | | | L81V, M146T |
| PS1489 | 0.4 | 0.4 | 0.4 | | PS1258 + V29A, L32M, H78N, L81F, T98A, W124G, |
| | | | | | R143C, M146Q |
| PS1490 | 0.3 | 0.4 | 0.3 | | PS1258 + S25A, V29A, L32F, Q64R, H78Q, L81V, |
| | | | | | Y118C, M146Q |
| PS1491 | 0.3 | 0.3 | 0.4 | | PS1258 + Q64K, P72F, V73L, H78N, L81V |
| PS1492 | 0.3 | 0.4 | 0.4 | | PS1258 + V29L, W30F, L32M, Q64R, V73L, L81F |
| PS1493 | 0.3 | 0.3 | 0.4 | | PS1258 + V29A, L32M, P72I, V73W, L81I, M146N |
| PS1494 | 0.5 | 3.1 | 0.3 | | PS1258 + P72F, V73L, H78N, L81V |
| PS1495 | 0.6 | 8.9 | 0.4 | | PS1258 + S25A, V29A, L32M, Q64R, V73I, L81I |
| PS1496 | 0.3 | 0.4 | 0.3 | | PS1258 + S25A, V29I, L32A, E34V, A49V, P72F, V73L, |
| | | | | | H78N, L81I, F91L |
| PS1497 | 0.4 | 3.9 | 0.5 | | PS1258 + V29A, L32M, L81V |
| PS1498 | 0.4 | 0.4 | 0.3 | | PS1258 + S25A, V29I, L32A, E34V, A49V, P72F, V73L, |
| | | | | | H78N, L81I, Y108C |
| PS1499 | 0.3 | 0.3 | 0.2 | | PS1258 + S25A, V29I, L32A, E34V, A49V, P72F, V73L, |
| | | | | | H78N, L81I |
| PS1633 | 0.1 | 0 | 0 | | PS1259 + H78R, H145R, M146L |
| PS1634 | 0.8 | 0.8 | 0.8 | | PS1259 + W75K, H78A |
| PS1635 | 0.7 | 0.7 | 0.7 | | PS1259 + D76F, H78S |
| PS1636 | 0.9 | 0.9 | 0.8 | | PS1259 + W75I, D76F |
| PS1637 | 1 | 7.1 | 0.8 | | PS1259 + 174K |
| PS1638 | 0.7 | 6 | 0.5 | | PS1259 + C42N |
| PS1639 | 0.8 | 6.4 | 0.7 | | PS1259 + I80C |
| PS1640 | 1 | 6.4 | 0.8 | | PS1259 + L95N |
| PS1641 | 0.7 | 6.8 | 0.5 | | PS1259 + E192Q |
| PS1642 | 0.1 | 5.6 | 0 | | PS1259 + A166G |
| PS1643 | 0.7 | 6.4 | 0.5 | | PS1259 + A112T |
| PS1644 | 0.8 | 1.2 | 0.7 | | PS1259 + A112T, H145R, M146L |
| PS1645 | 0 | 0 | 0 | | PS1259 + C42N, D76F, H78A |
| PS1646 | 0.7 | 1.5 | 0.7 | | PS1259 + C42N, H145R, M146L |
| PS1647 | 0.9 | 1 | 0.8 | | PS1259 + D76F, A112T |
| PS1648 | 0.7 | 0.7 | 0.6 | | PS1259 + D76F, A112T, E192Q |
| PS1649 | 1 | 0.9 | 0.8 | | PS1259 + D76F, A166G |
| PS1650 | 0 | 0 | 0 | | PS1259 + D76F, H78A, A112T |
| PS1651 | 0 | 4.3 | 0 | | PS1259 + H145R, A166G |
| PS1652 | 0 | 0.5 | 0 | | PS1259 + H145R, M146L |
| PS1653 | 0.4 | 1.3 | 0.3 | | PS1259 + H145R, M146L, A166G |
| PS1654 | 0.8 | 1.8 | 0.8 | | PS1259 + H145R, M146L, E192Q |
| PS1655 | 0 | 3 | 0 | | PS1259 + M146L, A166G |
| PS1656 | 0.9 | 0.9 | 0.8 | | PS1258 + D76F |
| PS1737 | 4.3 | 6.3 | 0.8 | | PS1259 + K65R |
| PS1738 | 5.3 | 6.7 | 0.6 | | PS1259 + A122R |
| PS1739 | | | | | PS1259 + N120L |
| PS1740 | 2.7 | 5.8 | 0.8 | | PS1259 + C23Y |
| PS1741 | 3.9 | 5.9 | 0.7 | | PS1259 + V47F |
| PS1742 | 3.4 | 4.9 | 0.8 | | PS1259 + S22F |
| PS1743 | 5.1 | 6.8 | 0.4 | | PS1259 + K65R, A122R |
| PS1744 | 4.5 | 6.5 | 0.7 | | PS1259 + A12T, K65R |
| PS1745 | 3.8 | 5.4 | 0.8 | | PS1259 + A12T, S22F |
| PS1746 | 3.8 | 5.4 | 0.7 | | PS1259 + S22P |
| PS1747 | 3.5 | 5.4 | 0.5 | | PS1259 + I11V |
| PS1748 | 4.2 | 6.1 | 0.9 | | PS1259 + A12L |
| PS1749 | 5 | 7 | 0.7 | | PS1259 + P131R |
| PS1750 | 4.6 | 6.6 | 0.7 | | PS1259 + S66V |
| PS1751 | 5.4 | 6.8 | 0.9 | | PS1259 + E71R |
| PS1752 | 4.4 | 6.4 | 0.5 | | PS1259 + C85T |
| PS1753 | 5.3 | 7.2 | 0.4 | | PS1259 + D84A |
| PS1754 | 5.3 | 7.1 | 0.7 | | PS1259 + E70G |
| PS1755 | | | | | PS1259 + E70N |
| PS1756 | 3.9 | 5.7 | 0.6 | | PS1259 + S39Q |
| PS1757 | 4.4 | 6.3 | 0.6 | | PS1259 + C85P |
| PS1758 | 5.4 | 7.3 | 0.2 | | PS1259 + G67C, A122R |
| PS1821 | 2.1 | 5.2 | 0.4 | | PS1259 + S25T |
| PS1822 | 0.9 | 1 | 0.8 | | PS1259 + S25Y |
| PS1823 | 3.4 | 5.4 | 0.1 | | PS1259 + R56Q |
| PS1824 | 2 | 4.5 | 0 | | PS1259 + R56L |
| PS1825 | 1.3 | 3.8 | 0 | | PS1259 + R56I |
| PS1826 | 2.6 | 5.4 | 0.2 | | PS1259 + K57N |
| PS1827 | 2.9 | 4.8 | 0 | | PS1259 + K57L |
| PS1828 | 0.6 | 2.5 | 0 | | PS1259 + K57E |
| PS1829 | 2.9 | 5 | 0 | | PS1259 + R154Q |
| PS1830 | 3.5 | 5.5 | 0.1 | | PS1259 + R154L |
| PS1831 | 4 | 6.3 | 0.1 | | PS1259 + R154C |
| PS1832 | 1.5 | 6.2 | 0.8 | | PS1259 + S25C |
| PS1833 | 4.8 | 6.5 | 0.1 | | PS1259 + A12L, K65R, N120R |
| PS1834 | 2.6 | 5.7 | 0.1 | | PS1259 + A12T, C23F, S66V |
| PS1835 | 2.3 | 4.5 | 0.2 | | PS1259 + P13L, R41L, G67C |
| PS1836 | 4.4 | 6.4 | 0.2 | | PS1259 + A12L, K65R |
| PS1837 | | | | | PS1259 + S39Q, G67C, C23Y |
| PS1838 | | | | | PS1259 + N120L, V19I, P13L |
| PS1839 | 6.4 | 7.7 | 0.8 | | PS1259 + S39Q, C85T, N120R |
| PS1840 | 1.7 | 4.9 | 0 | | PS1259 + C23F |
| PS1841 | 2.7 | 4.1 | 0 | | PS1259 + R41L |
| PS1842 | 1.1 | 1.5 | 1.4 | | PS1259 + Q78R, D96N |
| PS1843 | 1.3 | 1.4 | 1.5 | | PS1259 + Q78R |
| PS1844 | 1.1 | 6.3 | 1.1 | | PS1259 + S22P, S25G, V29I, K31H, E34Q, V73I, Q78H |
| PS1845 | 0.9 | 1.5 | 1.5 | | PS1259 + Q78K, A149S |
| PS1846 | 0.1 | 0.7 | 0.1 | | PS1259 + I74F, D96N |
| PS1847 | 1.2 | 2 | 1.4 | | PS1259 + C23Q, D96N |
| PS1848 | 0.7 | 3 | 0.8 | | PS1259 + D96N, S150W |
| PS1849 | 0.1 | 0.1 | | 0 | PS1259 + E27V, L81A, D96N, A112T, A149D |
| PS1850 | 0.1 | 0.1 | | 0.1 | PS1259 + E27G, C42N, D46N, Q78W, A112T |
| PS1851 | 0.1 | 0 | | 0 | PS1259 + E26W, C42N, I80G, A112T, D148L |
| PS1852 | 1.3 | 5.2 | 2 | | PS1259 + D96N, S150R |
| PS1853 | 3.3 | 4.8 | 0.8 | | PS1259 + V79A |
| PS1854 | 0 | 0 | 0 | | PS1259 + Y24T, E26K, D76F, D96N, S150A |
| PS1855 | | | 1.1 | 0.7 | PS1248 + S24T, S25A, S28G, V32I, K34H, E37Q, V76I, |
| | | | | | Q81H, V83L |
| PS1856 | | | 0.6 | 0.6 | PS1248 + S24T, S25A, S28A, V32I, K34H, E37Q, V76I, |
| | | | | | Q81H, P103L |
| PS1857 | | | 0.1 | 0 | PS1248 + Q81R, H123L |
| PS1858 | | | 0.4 | 0.5 | PS1248 + C21G, E49K, W78R |
| PS1859 | | | 0 | 0 | PS1248 + F94L, N57S, Q81R |
| PS1860 | | | 0 | 0 | PS1248 + A115T, K117E, Q81R |
| PS1861 | | | 0.1 | 0.1 | PS1248 + A115T, E49G, P124S |
| PS1862 | | | 0.1 | 0.1 | PS1248 + S25P, S28G, V76I, Q81H |
| PS1863 | | | 0.1 | 0.1 | PS1248 + S28R, M61I, K117M |
| PS1864 | | | 0 | 0.1 | PS1248 + Q44L, R157L |
| PS1865 | | | 0.1 | 0 | PS1248 + Q81R |
| PS1866 | | | 0.3 | 0.1 | PS1259 + D96N, A149G |
| PS1867 | | | 1.3 | 1.1 | PS1259 + D96N, A149V |
| PS1868 | | | 0.3 | 0.1 | PS1259 + I74F, D96N, K147R |
| PS1869 | | | 1.5 | 0.6 | PS1259 + P72A, D96N |
| PS1870 | | | | | PS1259 + C23G, I74F, D96N, K147R |
| PS1871 | | | 1.1 | 0.9 | PS1259 + C23G, I74W, D96N |
| PS1872 | | | 0.8 | 0.6 | PS1259 + C42N, V73E, D96N, A112T |
| PS1873 | | | 0.2 | 0.1 | PS1259 + I74F, D96N, A149D |
| PS1874 | | | 0.1 | 0 | PS1259 + I74W, D96D, D148N |
| PS1875 | 0.1 | 0.8 | 1 | 0.2 | PS1259 + Q78K |
| PS1876 | | | 1.1 | 1 | PS1259 + I74W, D96N, A149E |
| PS1877 | | 4 | 0.1 | | PS1259 + K114R, R154C |
| PS1878 | | 3.1 | 0.2 | | PS1259 + S22F, C23Y |
| PS1879 | | 0.9 | 0.3 | | PS1259 + S22F, C23Y, E71R |
| PS1880 | | 5.3 | 0.3 | | PS1259 + C23Y, N120P |
| PS1881 | | 6.1 | 0.5 | | PS1259 + N120P, A122R, A149P |
| PS1882 | | 4.8 | 0.3 | | PS1259 + G67C |
| PS1883 | | | | | PS1259 + S22T, W30Y, K31H |
| PS1884 | | | | | PS1259 + A149V |
| PS1885 | | 3.3 | 0.1 | | PS1259 + I74F |
| PS1886 | | 5 | 0.5 | | PS1259 + I80V |
| PS1887 | | 4.8 | 0.1 | | PS1259 + P72C |
| PS1888 | | | | | PS1259 + S25G |
| PS1889 | | 4.6 | 0.5 | | PS1259 + V73C |
| PS1890 | | 3.3 | 0.1 | | PS1259 + A149C |
| PS1891 | | 4.3 | 0.1 | | PS1259 + A149R |
| PS1892 | | 4.4 | 0.4 | | PS1259 + S22E |
| PS1893 | | 0.5 | 0.1 | | PS1259 + V73E |
| PS1894 | | 2.4 | 0.1 | | PS1259 + W75Y |
| PS1895 | | 3.6 | 0 | | PS1259 + I74L, A149G |
| PS1896 | | 4.6 | 0.2 | | PS1259 + I74V, A149E |
| PS1897 | | 5.8 | 0.7 | | PS1259 + P72R, A149Q |
| PS1898 | | 5.1 | 0.2 | | PS1259 + S144V |
| PS2014 | 0.1 | 1.1 | 2 | | PS1259 + Q78K, S150R |
| PS2015 | 0.5 | 9.5 | 0.2 | | PS1259 + S25G, S150R |
| PS2016 | 0.2 | 0.3 | 1.3 | | PS1259 + C23Q, Q78K |
| PS2017 | 0.2 | 5.6 | 0.2 | | PS1259 + C23Q, S25G |
| PS2018 | 0.2 | 4.4 | 0.3 | | PS1259 + S25G, Q78K |
| PS2019 | 0.1 | 0.4 | 0.5 | | PS1259 + S22P, Q78K |
| PS2020 | 0.1 | 0.5 | 0.9 | | PS1259 + V73I, Q78K |
| PS2021 | 0.3 | 6.2 | 0.2 | | PS1259 + S25G, A149S |
| PS2022 | 0.1 | 0.2 | 0.2 | | PS1259 + Q78K, A149G |
| PS2023 | 0.2 | 0.3 | 0.6 | | PS1259 + Q78K, A149V |
| PS2024 | 0.1 | 0.2 | 0.4 | | PS1259 + Q78K, A149D |
| PS2025 | 0.1 | 0.3 | 0.6 | | PS1259 + Q78K, A149E |
| PS2026 | 0.2 | 3.3 | 0.2 | | PS1259 + S22P, S25G, Q78K |
| PS2027 | 0.2 | 2.7 | 0.2 | | PS1259 + S25G, Q78K, A149S |
| PS2028 | 0.2 | 4.8 | 0.4 | | PS1259 + S25G, Q78K, S150R |
| PS2029 | 0.3 | 0.9 | 1.2 | 0.4 | PS1259 + K31H, E34Q, Q78K |
| PS2030 | 0.3 | 5.4 | 1 | | PS1259 + S22P, S25G, V73I, Q78K |
| PS2031 | 0.4 | 4.7 | 1.2 | | PS1259 + S25G, K31H, E34Q, Q78K |
| PS2032 | 0.2 | 5.6 | 0.4 | | PS1259 + S22P, S25G, V29I, V73I, Q78K |
| PS2033 | 0.3 | 4.4 | 1 | | PS1259 + S22P, S25G, K31H, E34Q, Q78K |
| PS2034 | 0.1 | 3.2 | 0.2 | | PS1259 + S25G, V29I, K31H, E34Q, V73I, Q78K |
| PS2035 | 0.2 | 4.8 | 0.5 | | PS1259 + S22P, S25G, V29I, K31H, E34Q, V73I, Q78K |
| PS2036 | | 6.5 | 0.1 | 0.2 | PS1259 + I11V, S22P, S25G, C33A, E34Q, V73L, I74V, |
| | | | | | Q78H, I80L |
| PS2037 | | 8.8 | 0.2 | 0.1 | PS1259 + S22A, S25G, C33A, E34Q, V73I, I74V, Q78H, |
| | | | | | I80L |
| PS2038 | | 6.6 | 0.1 | 0 | PS1259 + S22A, S25G, K31H, E34G, V73I, Q78H, I80L |
| PS2039 | | 6.9 | 0.1 | 0 | PS1259 + S22A, S25G, V29I, K31H, C33A, E34Q, Q78H |
| PS2040 | | 6.1 | 0 | 0 | PS1259 + S22P, S25A, W30Y, C33A, V73I |
| PS2041 | | 6.7 | 0.1 | 0 | PS1259 + S22P, S25G, V29I, K31H, C33A, E34Q, |
| | | | | | Q78H, I80L |
| PS2042 | | 6.1 | 0.1 | 0 | PS1259 + S22P, S25G, V29I, K31H, C33A, E34Q, V73I, |
| | | | | | I74L, Q78H |
| PS2043 | | 5.6 | 0.1 | 0 | PS1259 + S22P, S25G, V29I, K31H, V73I, I74L, Q78H |
| PS2044 | | 5.9 | 0.1 | 0 | PS1259 + S22P, S25G, V29I, K31H, E34Q, V73I, I74L, |
| | | | | | Q78H |
| PS2045 | | 6 | 0.1 | 0.1 | PS1259 + S22P, S25G, W30Y, K31H, V73I, I74V, Q78H |
| PS2046 | | 5.6 | 0.2 | 0.1 | PS1259 + S22A, S25G, E34Q, V73I, I74L, Q78H |
| PS2047 | | 0.2 | 0.1 | 0.2 | PS1259 + F91L, N54S, Q78R |
| PS2048 | | 0.3 | 0.3 | 0.3 | PS1259 + A112T, K114E, Q78R |
| PS2049 | | 4.3 | 0.1 | 0.1 | PS1259 + A112T, D46G, P121S |
| PS2050 | | 6.5 | 0.1 | 0.2 | PS1259 + S22P, S25G, V73I, Q78H |
| PS2051 | | 0.3 | 0.3 | 0.3 | PS1259 + S25R, M58I, K114M |
| PS2052 | | 4 | 0 | 0.1 | PS1259 + R41L, R154L |
| PS2053 | | 8 | 0.1 | 0.1 | PS1259 + A12L, S22E, H35Y, K65R, E71R, P72V, |
| | | | | | L81M, A122R, P131R |
| PS2054 | | 5.9 | 0.1 | 0 | PS1259 + A12L, S22P, S39Q, S66V, 174W, N120R, |
| | | | | | A122R |
| PS2055 | | 7.4 | 0.1 | 0 | PS1259 + A12L, S22E, K65R, E71R, P72V, N120R, |
| | | | | | A122R |
| PS2056 | | 5.6 | 0 | 0.1 | PS1259 + A12L, S22P, S39Q, K65R, E71R, A122R |
| PS2057 | | 6.1 | 0.1 | 0 | PS1259 + A12L, S22E, N120R, A122R |
| PS2116 | | 0.9 | 2.5 | 0.8 | PS1259 + S22E, Q78K |
| PS2117 | | 3.5 | 3.2 | 0.9 | PS1259 + P72R, Q78K |
| PS2118 | | 1.4 | 2 | 0.8 | PS1259 + Q78K, A149Q |
| PS2119 | | 2.9 | 2.3 | 0.9 | PS1259 + Q78K, A149V |
| PS2120 | | 2.4 | 3.7 | 1.2 | PS1259 + S39Q, Q78K, C85T, N120R |
| PS2121 | | 2.5 | 3.5 | 1.3 | PS1259 + S22E, S39Q, Q78K, C85T, N120R |
| PS2122 | | 1.5 | 2.4 | 0.8 | PS1259 + S22E, Q78K, A149Q |
| PS2123 | | 2.4 | 2.2 | 1 | PS1259 + S22E, Q78K, N120R |
| PS2124 | | 1.2 | 2 | 0.6 | PS1259 + S22E, Q78K, C85T |
| PS2125 | | 1.2 | 2.1 | 0.8 | PS1259 + S22E, S39Q, Q78K |
| PS2126 | | 1.8 | 3.2 | 1.1 | PS1259 + Q78K, N120R, A149Q |
| PS2127 | | 1.3 | 2.2 | 0.8 | PS1259 + Q78K, C85T, A149Q |
| PS2128 | | 1.2 | 1.9 | 0.9 | PS1259 + S39Q, Q78K, A149Q |
| PS2129 | | 1.7 | 3.2 | 0.2 | PS1259 + S22E, Q78K, N120R, A149Q |
| PS2130 | | 1.6 | 2.4 | 0.9 | PS1259 + S22E, Q78K, C85T, A149Q |
| PS2131 | | 1.4 | 2.2 | 0.8 | PS1259 + S22E, S39Q, Q78K, A149Q |
| PS2132 | | 2.5 | 3.4 | 0.9 | PS1259 + S22E, Q78K, C85T, N120R |
| PS2133 | | 2.2 | 3.3 | 1.2 | PS1259 + S22E, S39Q, Q78K, N120R |
| PS2134 | | 2.1 | 3.5 | 0.4 | PS1259 + S22E, Q78K, N120R, A149V |
| PS2135 | | | | | PS1259 + S22E, S39Q, Q78K, C85T |
| PS2136 | | | | | PS1259 + S22E, Q78K, C85T, A149V |
| PS2137 | | | | | PS1259 + S22E, S39Q, Q78K, A149V |

Tables 16 and 17 show example results from experiments evaluating binding kinetics for Ntaq1-homologous variants identified above. Ensemble Rapid kinetics measurements were obtained for N-terminal N, Q, E, and D inherent binding by variants, with the highly pure unconjugated protein preps of top variants of hNTQ after high-throughput kinetics evaluation. Binding affinities (Kd) were determined by polarization at 20° C. (a dash indicates not measured). The kₒₙ rate constants and koff rates were derived by stopped-flow rapid kinetic analysis at 30° C. for NA, QA, EA, and DA (a dash indicates not measured). Table 18 shows a summary of mutations in these Ntaq1-homologous variants.

TABLE 16
Kinetics Study: Ntaq1-homologous variants (QA/NA peptides)

| | QA Kd ± | | | NA Kd ± | | |
|---|---|---|---|---|---|---|
| | std.error | QA | | std.error | NA | |
| Variant | (nM) | (kon/nM/s) | QA (koff/s) | (nM) | (kon/nM/s) | NA (koff/s) |
| PS1248 | 446 ± 27 | 0.001 | 1.7 | 4166 ± 349 | — | 6.3 |
| PS1252 | 3579 ± 768 | — | — | 4709 ± 1414 | — | — |
| PS1258 | 281 ± 52 | 0.001 | 0.5 | Very weak | — | — |
| PS1259 | 279 ± 20 | 0.002 | 1 | 1326 ± 44 | 0.002 | 4.1 |
| PS1260 | 2164 ± 892 | — | — | 2235 ± 786 | — | — |
| PS1751 | 197 ± 20 | — | — | 662 ± 39 | — | — |
| PS1844 | 90 ± 9 | — | — | 14547 ± 1208 | — | — |
| PS1845 | 10303 ± 392 | — | — | Weak | — | — |
| PS1848 | — | — | — | — | — | — |
| PS1852 | 5752 ± 667 | 0.002 | 1.03 | Weak | — | — |
| PS1875 | Very weak | — | — | Very weak | — | — |
| PS2020 | 12024 ± 1482 | — | — | Very weak | — | — |
| PS2029 | 14295 ± 1361 | — | — | Very weak | — | — |
| PS2121 | 3995 ± 137 | — | — | Very weak | — | — |
| PS2123 | 4628 ± 167 | — | — | Very weak | — | — |
| PS2132 | 2893 ± 91 | — | — | Very weak | — | — |

TABLE 17
Kinetics Study: Ntaq1-homologous variants (EA/DA peptides)

| | EA Kd ± | | | DA Kd ± |
|---|---|---|---|---|
| Variant | std. error (nM) | EA (kon/nM/s) | EA (koff/s) | std. error (nM) |
| PS1248 | — | — | — | — |
| PS1252 | — | — | — | — |
| PS1258 | — | — | — | — |
| PS1259 | — | — | — | — |
| PS1260 | — | — | — | — |
| PS1751 | 0 | — | — | — |
| PS1844 | 15119 ± 1216 | — | — | — |
| PS1845 | 10006 ± 672 | — | — | — |
| PS1848 | 12329 ± 619 | — | — | — |
| PS1852 | 21773 ± 5428 | — | — | — |
| PS1875 | 11242 ± 717 | 0.001 | 17.262 | — |
| PS2020 | 14358 ± 1671 | 0.002 | 10.74 | — |
| PS2029 | 12522 ± 1448 | — | 8.18 | — |
| PS2121 | 2570 ± 117 | 0.002 | 10.28 | 21951 ± 1942 |
| PS2123 | 3025 ± 140 | 0.002 | 8.5348 | 33054 ± 4895 |
| PS2132 | 2171 ± 69 | 0.003 | 10.27 | 19167 ± 2025 |

TABLE 18
Ntaq1-homologous variants

| Variant | Mutations (relative to SEQ ID NO: 3) |
|---|---|
| PS1248 | C28S, H81Q |
| PS1252 | C27S, H80Q, M148E |
| PS1258 | C25S |
| PS1259 | C25S, H78Q |
| PS1260 | C25S, H78Q, M146E |
| PS1751 | E71R |
| PS1844 | S22P, C25G, V29I, K31H, E34Q, V73I |
| PS1845 | H78K, A149S |
| PS1848 | D96N, S150W |
| PS1852 | D96N, S150R |
| PS1875 | H78K |
| PS2020 | V73I, H78K |
| PS2029 | K31H, E34Q, H78K |
| PS2121 | S22E, S39Q, H78K, C85T, N120R |
| PS2123 | S22E, H78K, N120R |
| PS2132 | S22E, H78K, C85T, N120R |

The high-throughput ensemble kinetics studies of ˜200 high-throughput preps and the extensive rapid kinetics of >20 large-scale preps described above identified the top mutations for each round of library design and constructs, which led to the identification of PS1259 (Q and N recognizer) and PS2132 (E recognizer).

Sequencing Performance with Glutamate Recognizer PS1875

Sequencing runs were performed using QP1165 (EIAFLKQRVWK (SEQ ID NO: 1084)) peptide, CDNF and VIME peptide libraries using a mixture of six recognizers, including the glutamate recognizer PS1875 (at 250 or 500 nM, labeled with a long-lifetime BODIPY dye). The E recognition was observed for QP1165, EFLNRFYK (SEQ ID NO: 1068) in CDNF and VELQEEIAFLK (SEQ ID NO: 1085) peptides in VIME libraries. An example trace is shown for each peptide in FIGS. 32B-32E. The 6th dye cluster separation is clearly seen in the cluster plot.

### Preparation of Glutamate Recognizer PS2132

The glutamate recognizer PS2132 was expressed, purified, and labeled for further evaluation in sequencing reactions.

FIGS. 32F-32G show example results from expression and purification of bis-biotinylated PS2132 in 2 L scale. FIG. 32F shows an AKTA purification Chromatogram of PS2132 with Cobalt ion affinity column showing PS2132 protein elution peak. FIG. 32G shows SDS-PAGE analysis of PS2132 expression, purification steps and Elution fractions (molecular weight marker is shown in kDa).

FIGS. 32H-32I show example results from size exclusion chromatography (SEC) of PS2132 labeled with streptavidin-linked long-lifetime BODIPY dye. FIG. 32H shows an SEC chromatograph of PS2132 labeled with long-lifetime BODIPY dye. Protein was detected at 280 nm and dye at 540 nm (SEC Buffer: 25 mM HEPES 150 mM KCl pH 8.0 and 0.01% Tween-20, at 0.5 mL/min; Instrument: Agilent 1260 Infinity LC System; Column: Agilent AdvanceBio SEC (7.8 mm×300 Å, 2.7 μm, Part No. 0006661476-15)). FIG. 32I shows SDS PAGE gel of SEC peaks collected in Top Panel (Peak 1-6). Lanes are numbered according to peaks collected in Top Panel. Samples in peak 2, 3 and 4 were pooled, concentrated and used for chip dynamic run. Peak samples with added Biotin are labelled as 1B-6B. Biotin binding to SV-dye was used to check if there was free SV dye present in the sample.

FIG. 32J shows example results from Quality Control SDS PAGE gel analysis of PS2132 labeled with long-lifetime BODIPY dye pre- and post-SEC column purification. Lanes 1 and 1B are pre SEC sample. Lanes 2 and 2B are SEC purified 1:1 PS2132-BODIPY complexes. Samples with added fluorescent Biotin (tagged with Alexa 647) are labeled 1B and 2B. Biotin binding to SV-dye was used to check if there was free SV dye present in the sample.

Sequencing Performance with Glutamate Recognizers PS2132, PS2121, PS2123

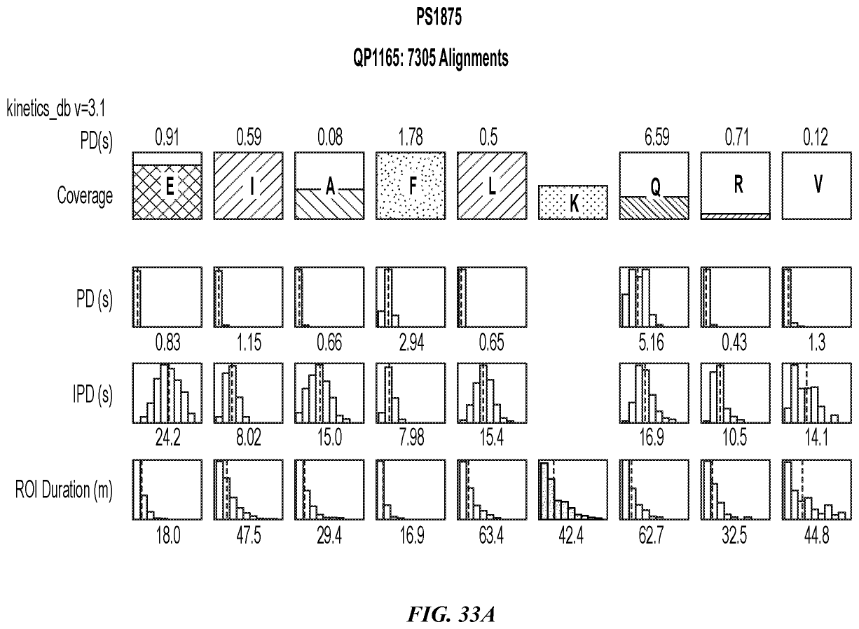

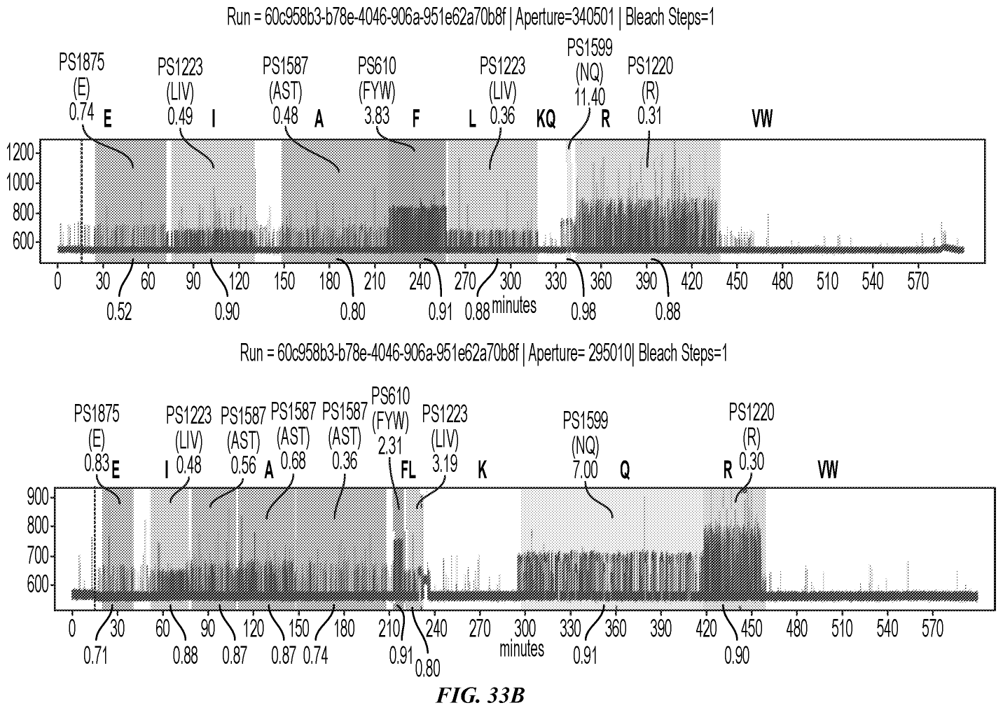

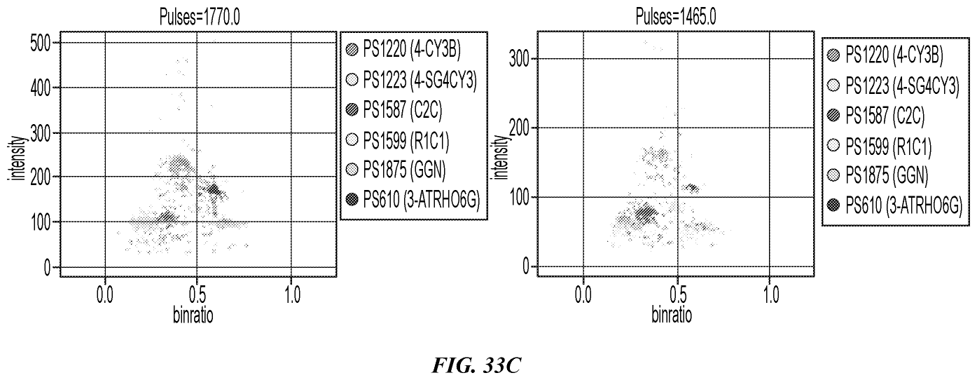

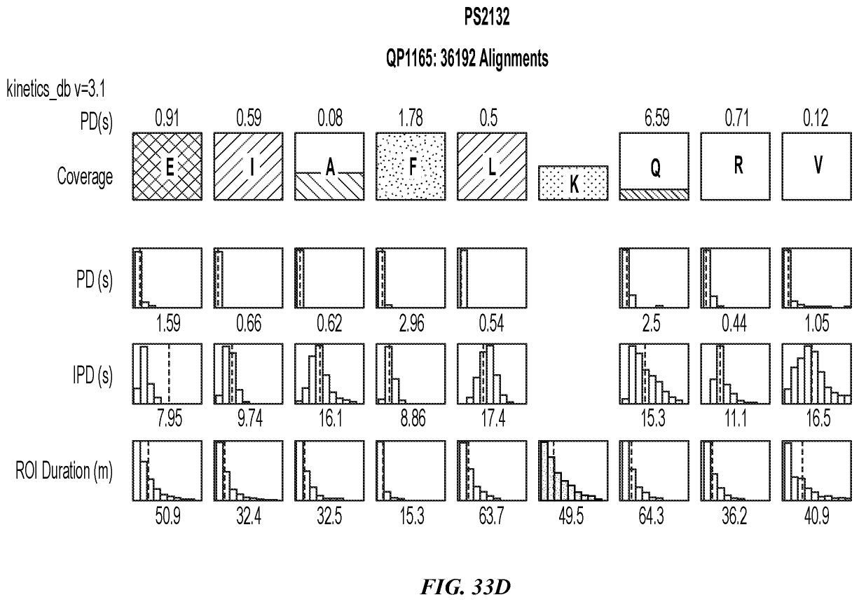

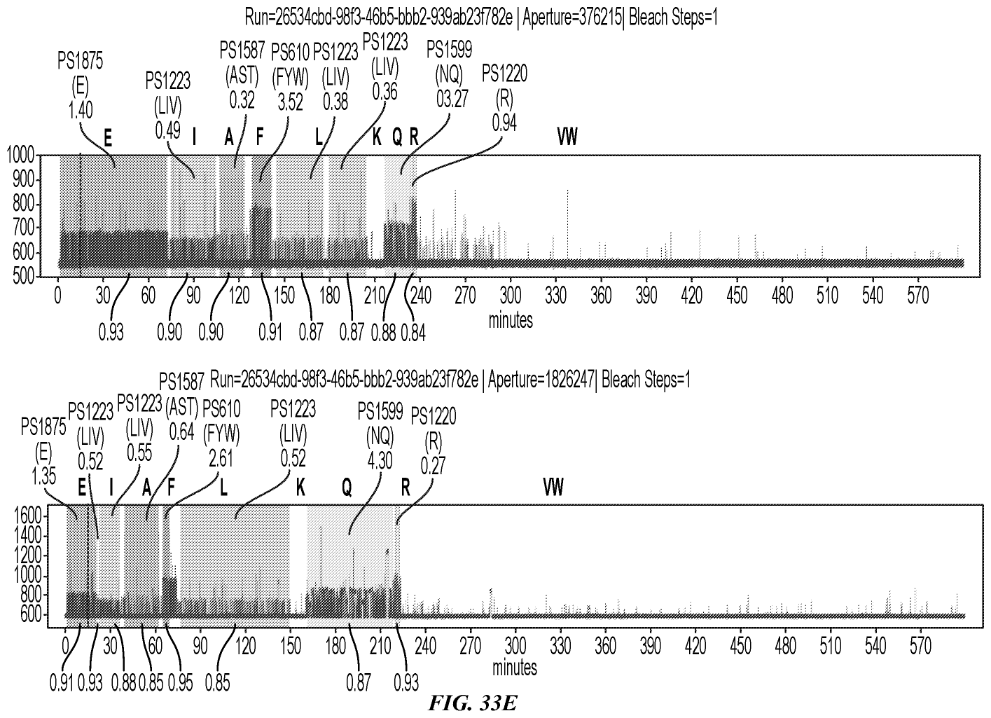

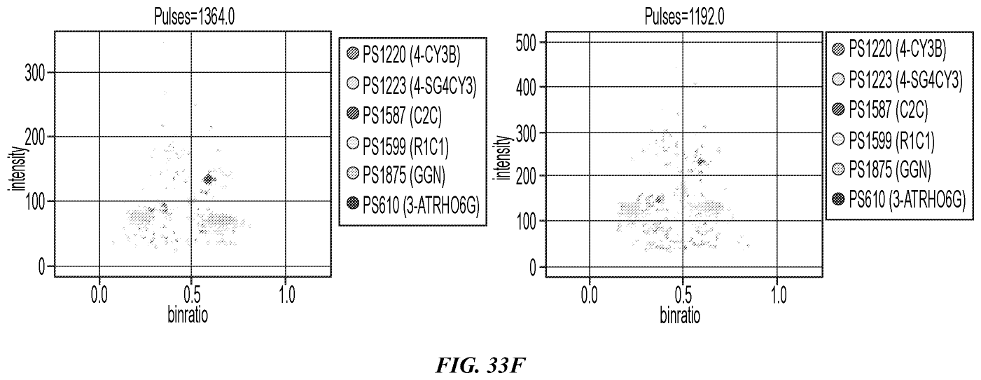

FIGS. 33A-33F show example results from on-chip recognition of E by PS1875 (FIGS. 33A-33C) and PS2132 (FIGS. 33D-33F) labeled with long-lifetime BODIPY dye. The candidates were compared at 500 nM for sequencing QP1165 peptide (EIAFLKQRVW (SEQ ID NO: 1084)) with the 5 recognizers on same chip (50 nM PS610, 125 nM PS1220, 75 nM PS1223, 250 nM PS1587 (tandem of PS1165) and 300 nM PS1599 (tandem of PS1259) with AP64/AP37). Kinetic signatures and parameters are shown at top, and example traces for both candidates are shown at bottom, showing recognition and pulsing for E the first residue. PD, IPD and number of alignments were improved in PS2132 compared to PS1875.

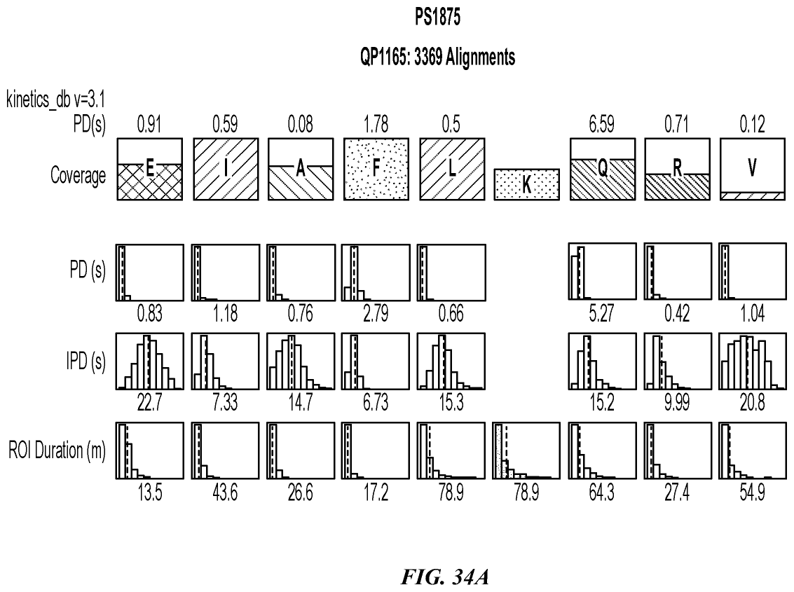

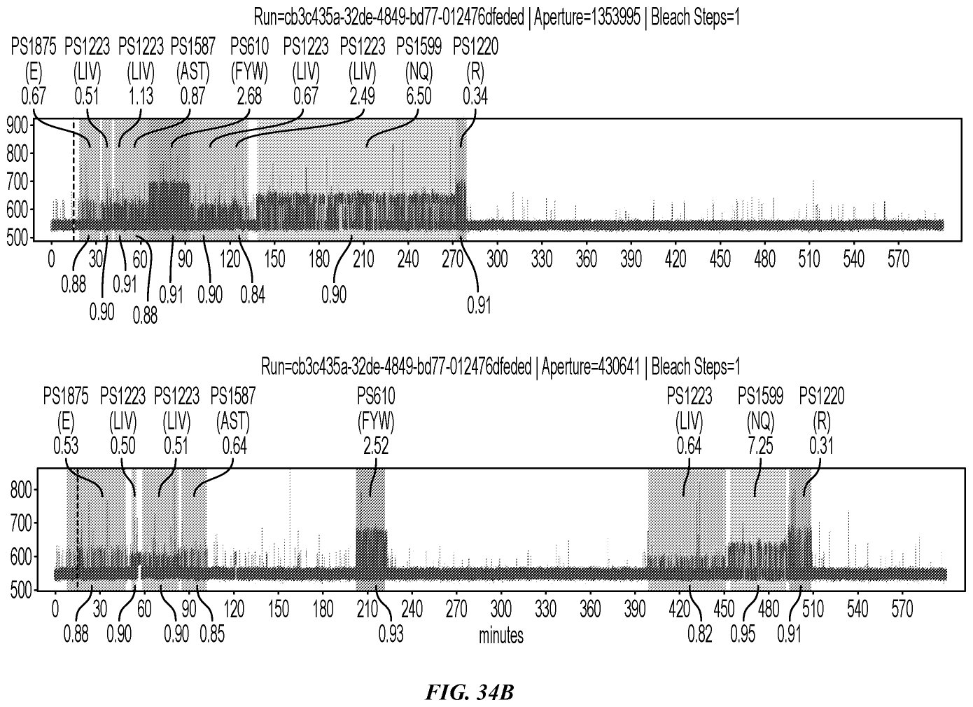

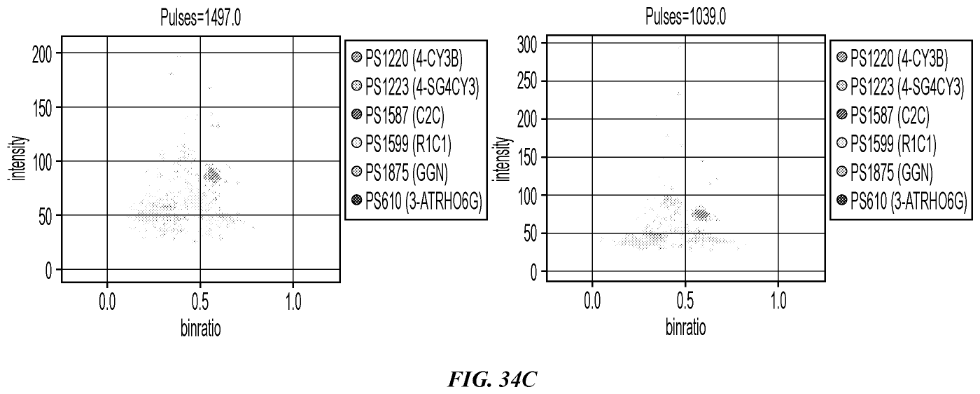

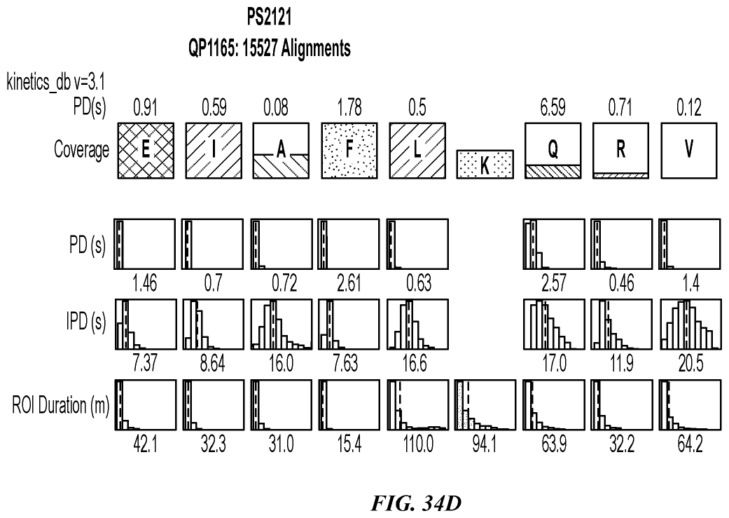

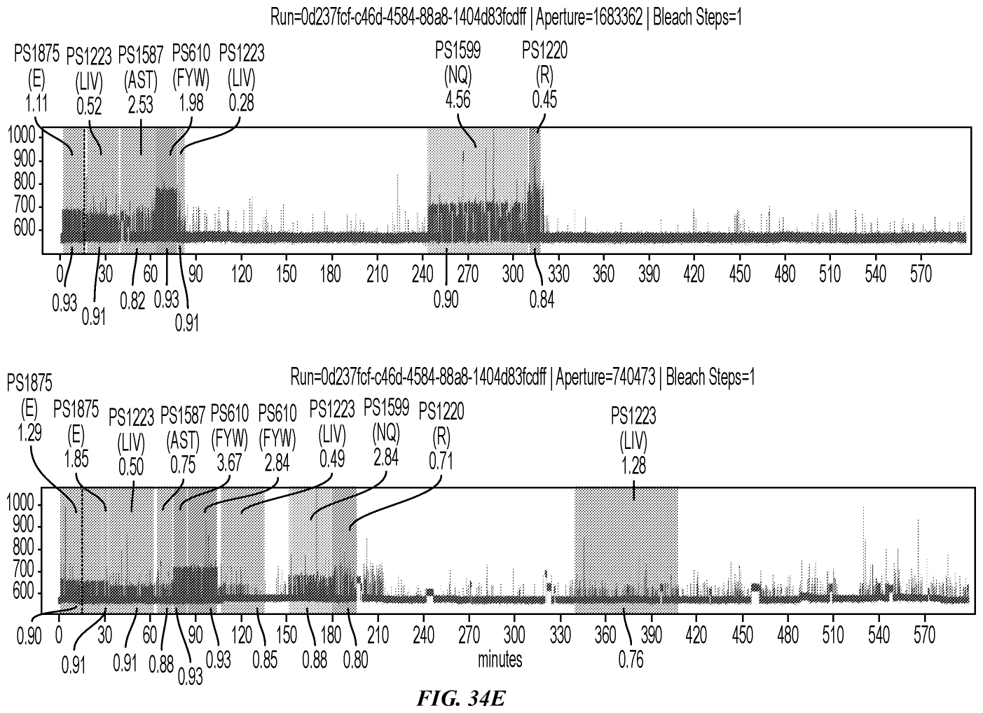

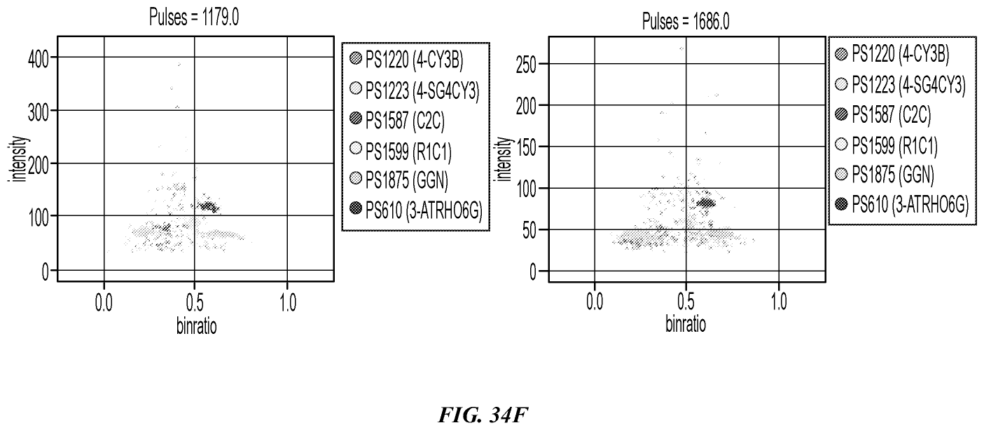

FIGS. 34A-34F show example results from on-chip recognition of E by PS1875 (FIGS. 34A-34C) and PS2121 (FIGS. 34D-34F) labeled with long-lifetime BODIPY dye. The candidates were compared at 500 nM for sequencing QP1165 peptide (EIAFLKQRVW (SEQ ID NO: 1068)) with the 5 recognizers on same chip. Kinetic signatures and parameters are shown at top, and example traces for both candidates are shown at bottom, showing recognition and pulsing for E the first residue. PD, IPD and number of alignments were improved in PS2121 compared to PS1875.

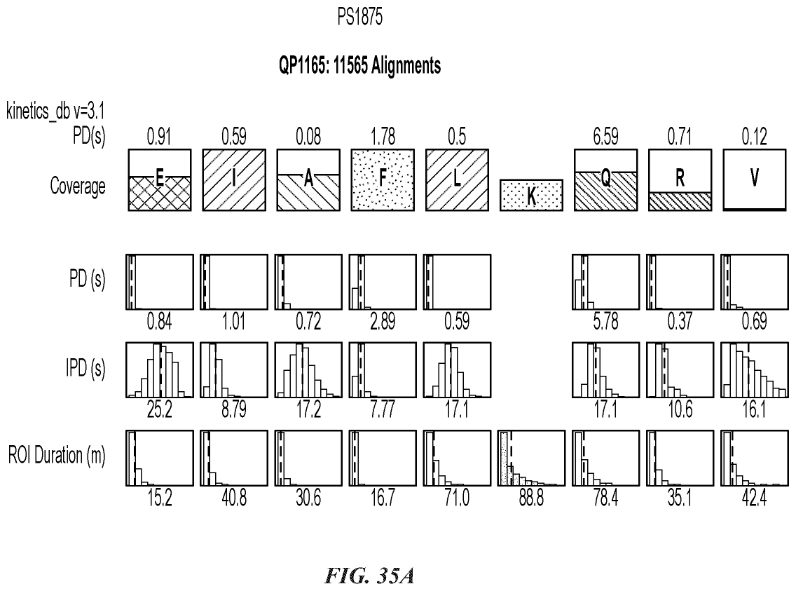

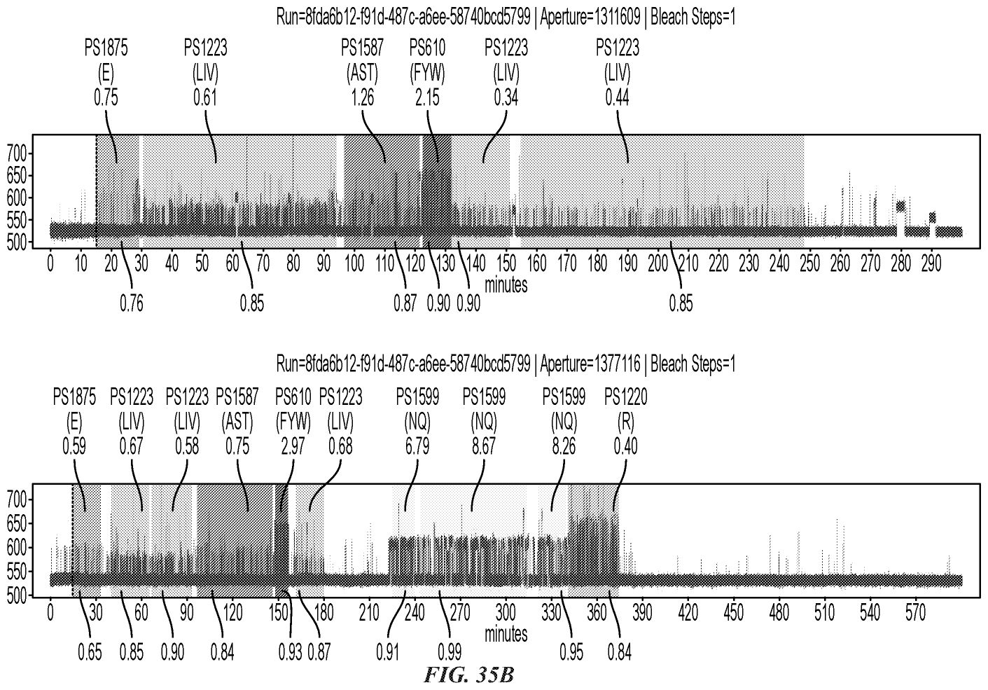

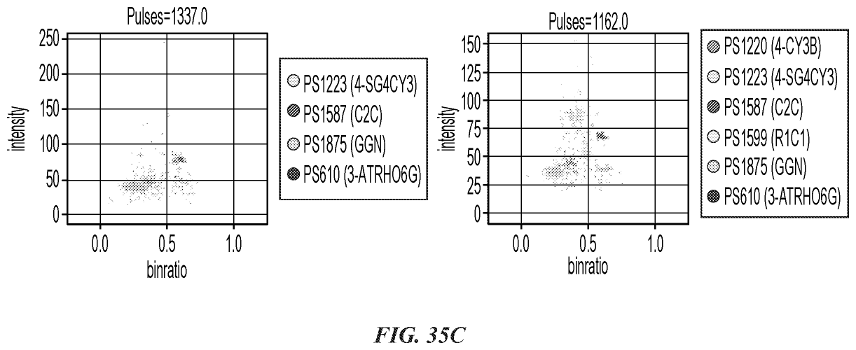

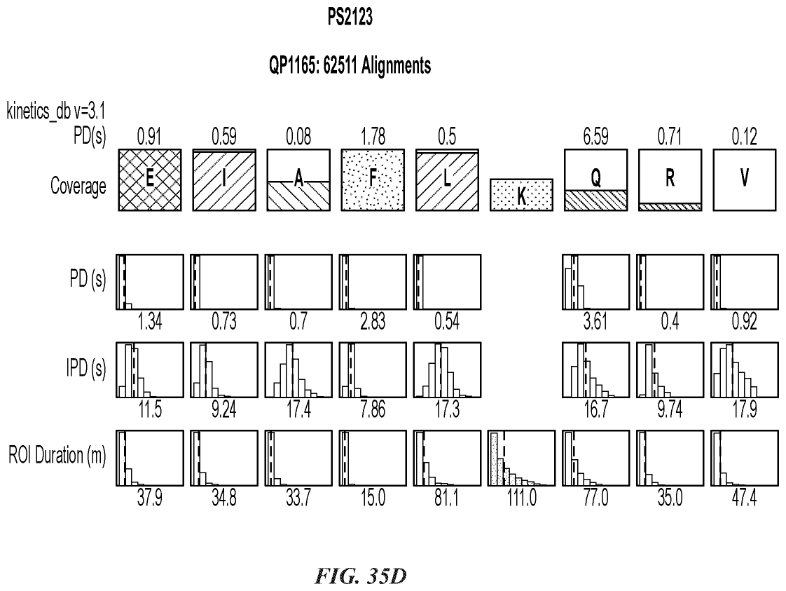

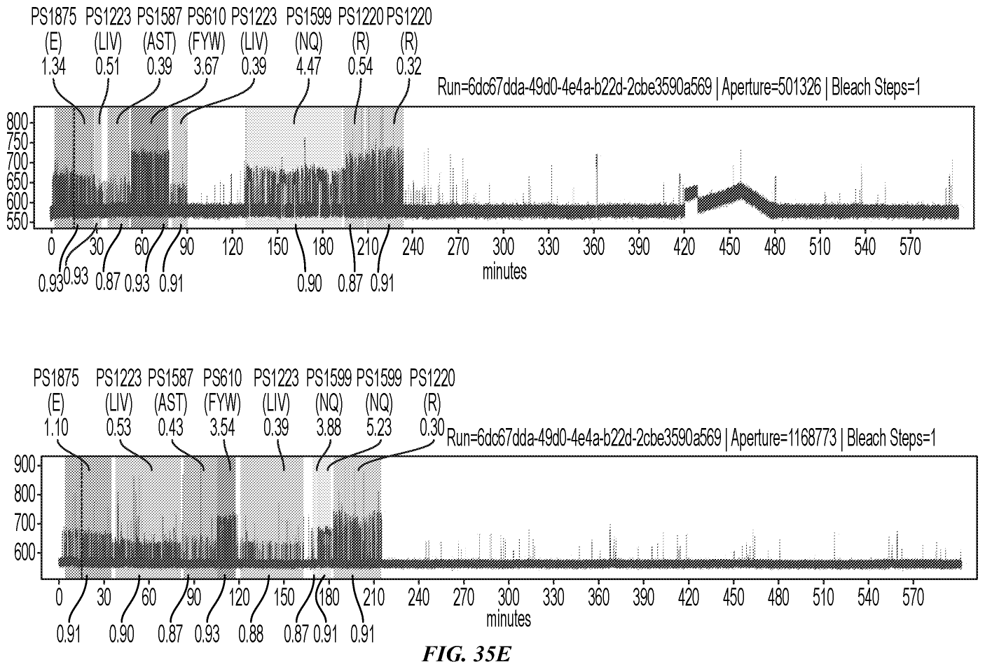

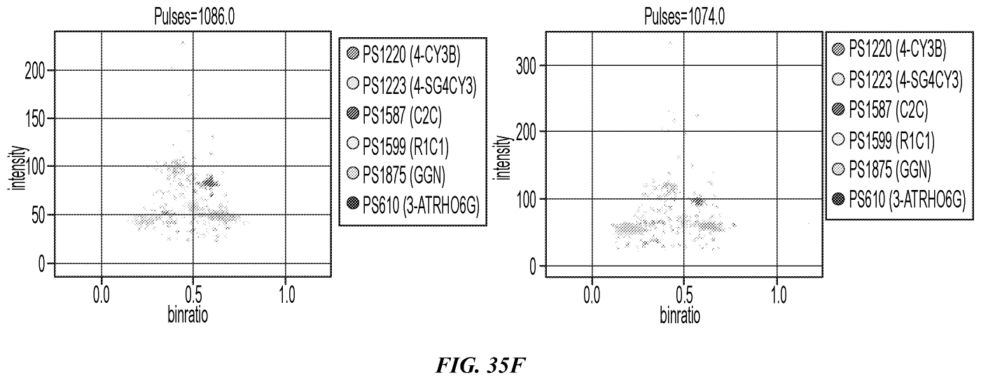

FIGS. 35A-35F show example results from on-chip recognition of E by PS1875 (FIG. 35A-35C) and PS2123 (FIG. 35D-35F) labeled with long-lifetime BODIPY dye. The candidates were compared at 500 nM for sequencing QP1165 peptide (EIAFLKQRVW (SEQ ID NO: 1086)) with the 5 recognizers on same chip. Kinetic signatures and parameters are shown at top, and example traces for both candidates are shown at bottom, showing recognition and pulsing for E the first residue. PD, IPD and number of alignments were improved in PS2123 compared to PS1875.

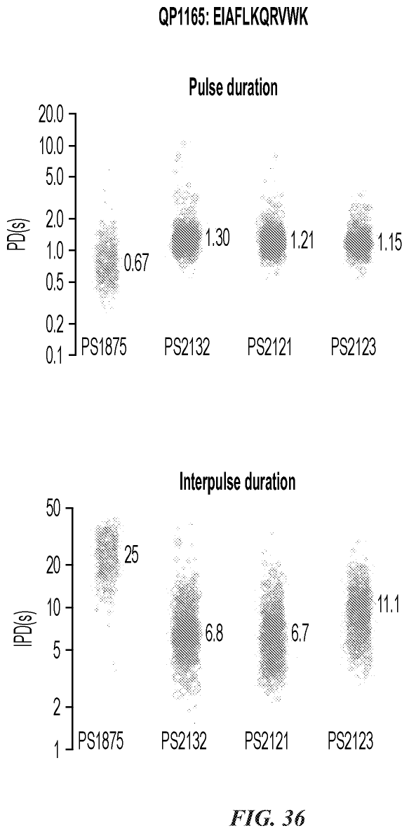

As described above, sequencing runs were performed using QP1165 (EIAFLKQRVWK (SEQ ID NO: 1084)) peptide with a mixture of 6 recognizers containing either PS1875, PS2132, PS2121, or PS2123 as the recognizer for glutamate (E). FIG. 36 shows pulse duration (top) and interpulse duration (bottom) of RSs corresponding to E recognition in aligned reads for each run, with median values indicated. PS2132, PS2121, and PS2123 displayed longer pulse duration and shorter interpulse duration compared to PS1875.

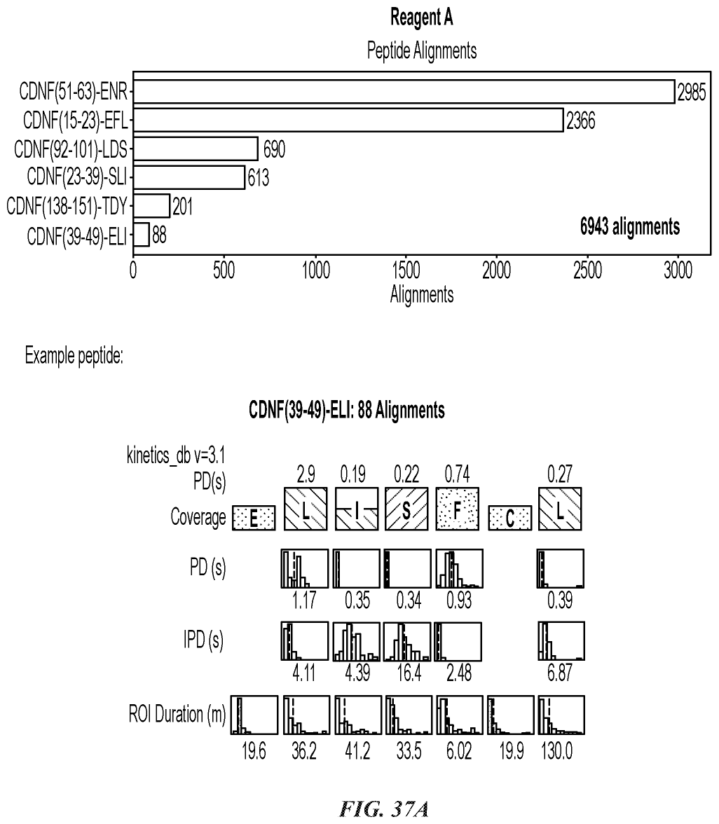

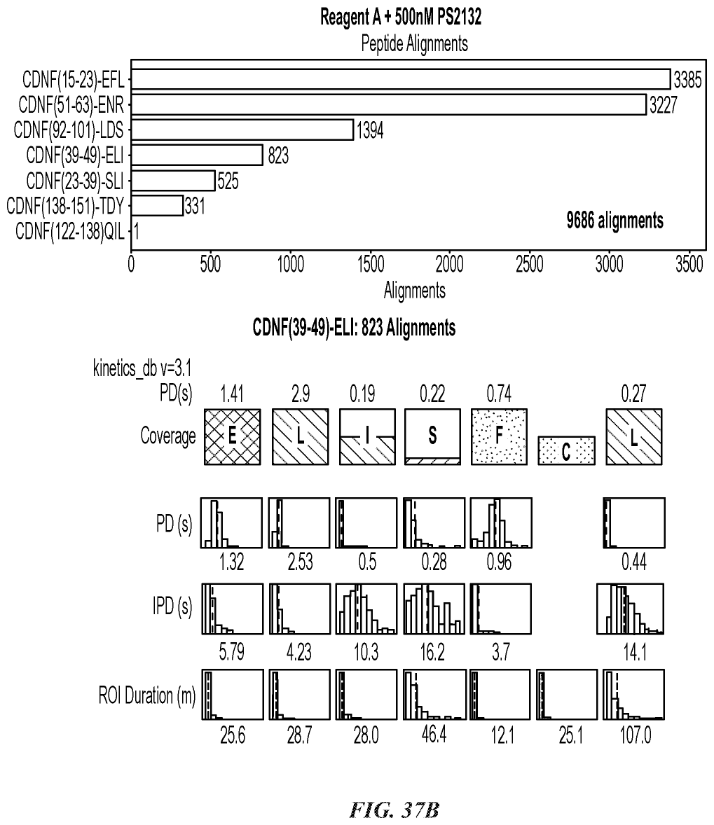

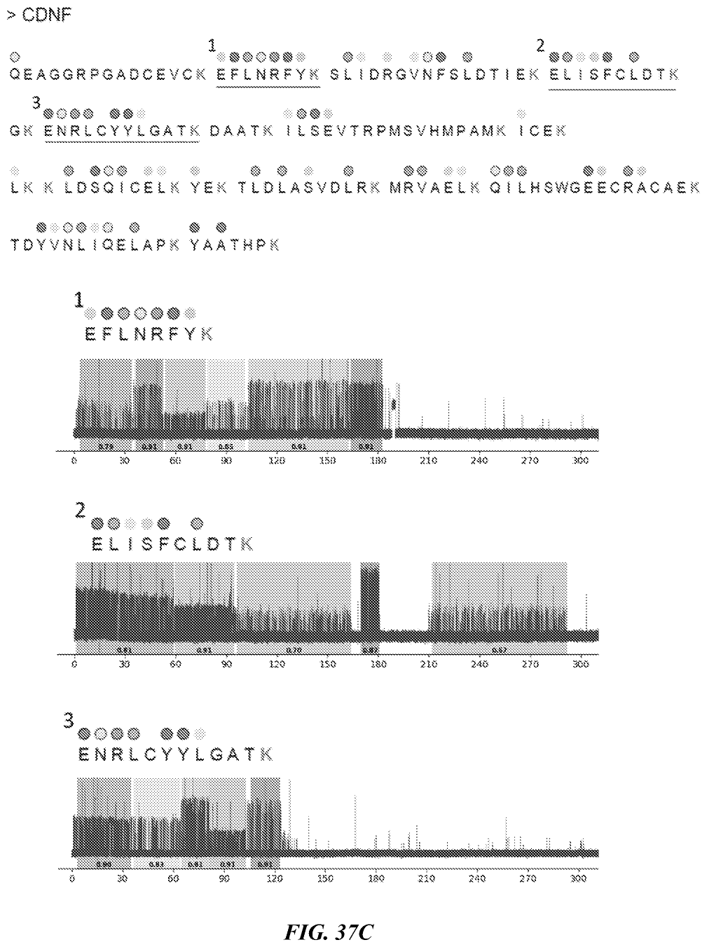

FIGS. 37A-37C show example results from sequencing runs of a CDNF library performed using reagent A containing a mixture of 5 recognizers (FIG. 37A) or reagent A combined with the E recognizer PS2132 (FIG. 37B). Runs containing PS2132 displayed 2.3-fold higher number of alignments (normalized by total number of active wells) compared to runs with reagent A only. Example peptide alignment distribution plots for CDNF are shown for each condition at top (FIGS. 37A and 37B). The peptide ELISFCLDTK (SEQ ID NO: 1069) displayed an increase in alignments from 88 to 832 alignments in an example set of runs due to the ability to detect E when PS2132 was added (shown at bottom, FIGS. 37A and 37B). FIG. 37C depicts further example results from CDNF peptide library sequencing using the mixture of 6 recognizers (including the E recognizer PS2132 in Reagent A). As shown by the example traces in FIG. 37C, robust E recognition was observed for 3 peptides: EFLNRFYK (SEQ ID NO: 1068), ELISFCLDTK (SEQ ID NO: 1069), and ENRLCYYLGATK (SEQ ID NO: 1070).

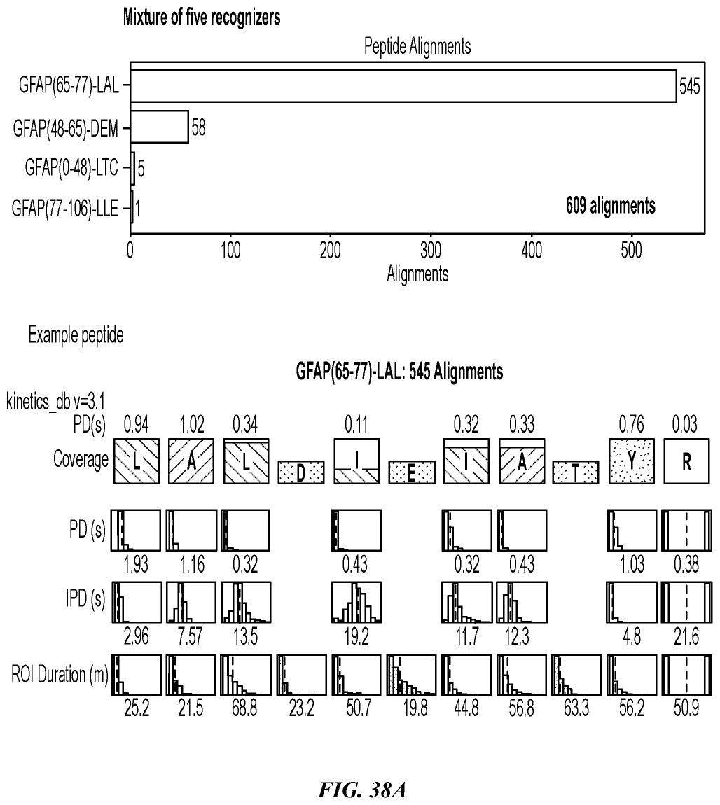

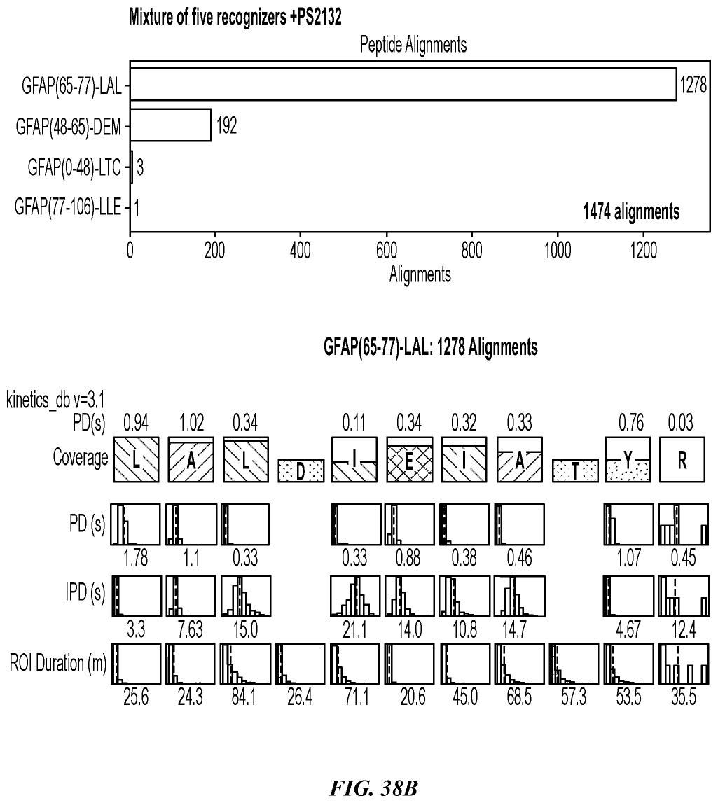

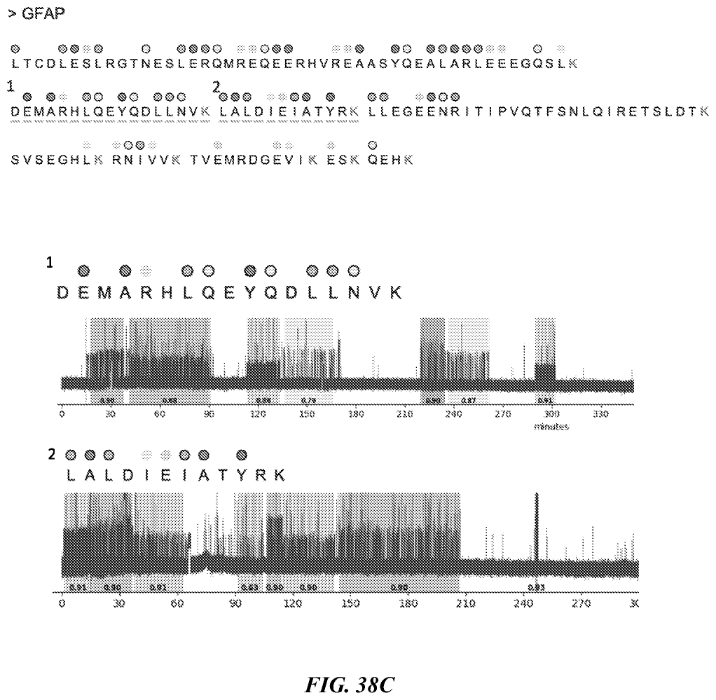

FIGS. 38A-38C show example results from sequencing runs performed on GFAP peptide library with (FIG. 38A) or without (FIG. 38B) recognizer for glutamate (E) PS2132 in combination with five recognizers. Runs containing PS2132 displayed 2-fold higher number of alignments (Normalized to total number of active wells) compared to runs with five recognizers. Example peptide alignment distribution plots for GFAP are shown for each condition at top (FIGS. 38A and 38B). The peptide LALDIEIATYRK (SEQ ID NO: 1072) displayed an increase in alignments from 545 to 1278 alignments in an example run due to the ability to detect E when PS2132 was added (shown at bottom, FIGS. 38A and 38B). FIG. 38C depicts further example results from GFAP peptide library sequencing using the mixture of 6 recognizers (including the E recognizer PS2132 in Reagent A). As shown by the example traces in FIG. 37C, robust E recognition was observed for 2 peptides: DEMARHLQEYQDLLNVK (SEQ ID NO: 1071) and LALDIEIATYRK (SEQ ID NO: 1072).

### PS2132: Computational Modeling

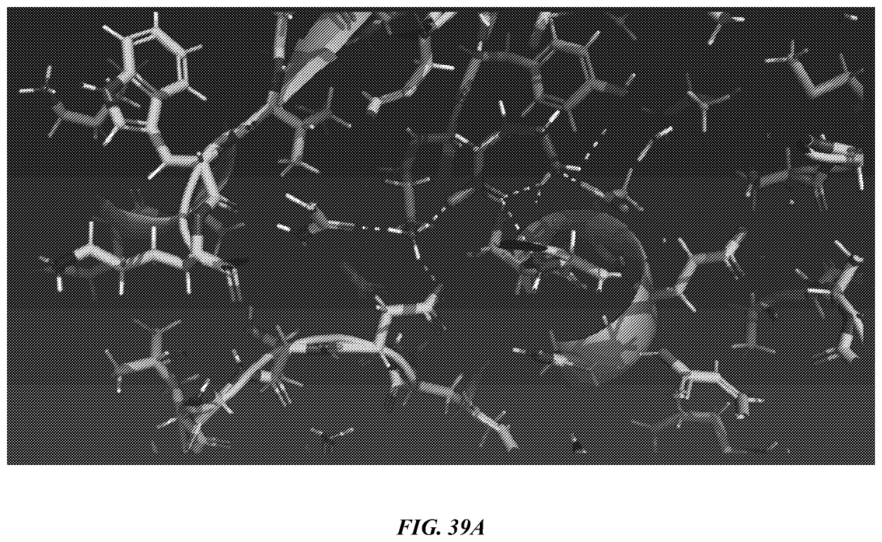

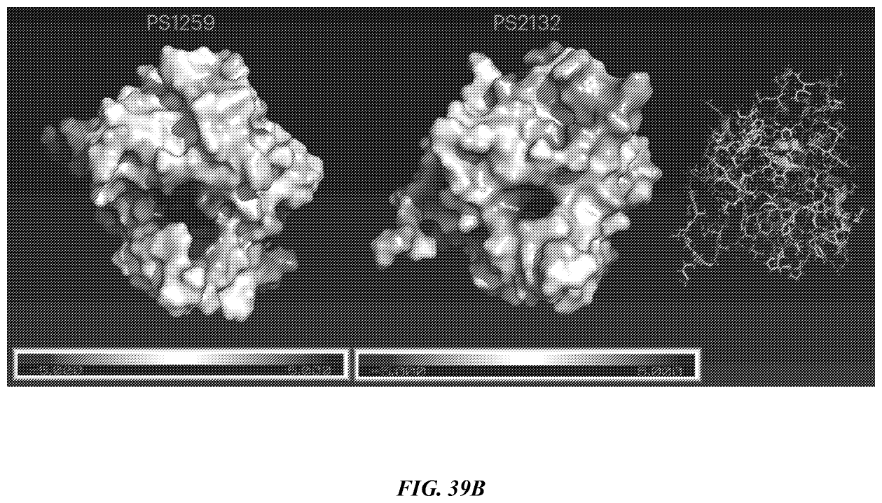

PS2132 was evaluated by computational modeling. FIG. 39A depicts the binding pocket of PS2132 in complex with N-terminal glutamate peptide (sticks). As shown by the hydrogen bonding depicted as dashed lines, the positively-charged side chain of lysine at position 78 (H78K) forms a hydrogen bond with the side chain of N-terminal glutamate, and the negatively-charged side chain of E26 forms a hydrogen bond with the backbone amine of N-terminal glutamate. The positively-charged lysine mutation in PS2132 (H78K) enables binding to the negatively-charged glutamate, as compared to the polar residue at the corresponding position in PS1259 (H78Q), which enhances glutamine binding (Example 8).

FIG. 39B depicts modeling of surface charge for PS1259 and PS2132. Based on the differences in surface charge and the glutamate binding properties of PS2132, the additional mutations S22E, C85T, and N120R make the overall surface charge more amendable to negative amino acid binding.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

1. A recombinant or synthetic amino acid binding protein having an amino acid sequence that is at least 80% identical to SEQ ID NO: 1, wherein the amino acid sequence comprises an amino acid substitution at one or more positions corresponding to E22, R31, L39, N41, D42, D43, D44, H45, T46, Y47, V50, Q55, P62, E63, L68, A69, V72, D73, Q75, Y100, and M111 of SEQ ID NO: 1.

2. The amino acid binding protein of claim 1, wherein the amino acid sequence comprises an amino acid substitution at a position corresponding to N41, and at one or more positions corresponding to E22, R31, L39, D42, H45, V50, Q55, P62, E63, L68, V72, Q75, Y100, and M111.

3. The amino acid binding protein of claim 1, wherein the amino acid sequence comprises an amino acid substitution at a position corresponding to N41 and at one or more positions corresponding to Q55, E63, L68, V72, and Y100.

4. The amino acid binding protein of claim 1, wherein the amino acid substitution is selected from E22V, R31H, L39M, N41D, D42 L/P, H45C/F, V50A/F/Y, Q55H/R, P62R, E63A/G/K/S, L68M, V72M, Q75 L, Y100R, and M111A/S.

5. The amino acid binding protein of claim 1, wherein the amino acid substitution is selected from N41D, Q55R, E63S, L68M, V72M, and Y100R.

6. The amino acid binding protein of claim 1, wherein the amino acid sequence is at least 85%, at least 90%, at least 95%, at least 98%, 80-98%, 80-95%, 80-90%, 85-95%, or 90-98% identical to SEQ ID NO: 1.

7. The amino acid binding protein of claim 1, wherein the amino acid sequence is at least 90% identical to a sequence selected from any one of PS635-645, PS731-732, PS759-766, PS769, PS795-870, PS896-912, PS918-1043, PS1048-1100, PS1124-1137, PS1141-1161, PS1175-1199, PS1203-1217, PS1222-1245, PS1277-1305, PS1321-1350, and PS1425-1448 (SEQ ID NOs: 22-27, 87-88, 115-122, 125, 151-226, 249-265, 271-390, 395-446, 470-483, 487-507, 521-545, 549-563, 568-591, 622-650, 664-693, and 768-791).

8. A recombinant or synthetic amino acid binding protein comprising a structure of Formula (I) or a structural equivalent thereof:
β1-α1-α2-β2-α3-β3 (I),
wherein:
each of β1, β2, and β3 is a beta-strand;
each of α1, α2, and α3 is an alpha-helix;
each instance of “-” is a loop; and
at least a portion of each of α1, α2, the loop between β1 and α1, and the loop between α3 and β3 form a binding pocket for an amino acid ligand, wherein the binding pocket comprises one or more of the following:
i) a volume of approximately 170 Å³,
ii) an electrostatic potential of −3.0 RTe_{c}⁻¹ or less,
iii) negatively charged side-chains in at least 35% of amino acids that form the binding pocket,
iv) a plurality of hydrogen bond acceptors configured to form one or more hydrogen bonds in the presence of the amino acid ligand, and
v) a plurality of van der Waals contact positions configured to form van der Waals interactions in the presence of the amino acid ligand.

9-42. (canceled)

43. A recombinant or synthetic amino acid binding protein having an amino acid sequence that is at least 80% identical to SEQ ID NO: 2, wherein the amino acid sequence comprises an amino acid substitution at one or more positions corresponding to G19, K26, S29, F30, D31, D32, T33, C34, V35, T47, G48, T53, T54, T57, E58, F59, N61, 163, D65, D68, E70, A71, H74, and T75 of SEQ ID NO: 2.

44-48. (canceled)

49. The amino acid binding protein of claim 43, wherein the amino acid sequence is at least 90% identical to a sequence selected from any one of PS1101-1122, PS1218-1221, and PS1351-1398 (SEQ ID NOs: 447-468, 564-567, and 694-741).

50. A recombinant or synthetic amino acid binding protein comprising a structure of Formula (II) or a structural equivalent thereof:
β1-α1-β2-α2-α3 (II),
wherein:
each of β1 and β2 is a beta-strand;
each of α1, α2, and α3 is an alpha-helix;
each instance of “-” is a loop; and
at least a portion of each of α2, the loop between β1 and α1, and the loop between β2 and α2 form a binding pocket for an amino acid ligand, wherein the binding pocket comprises one or more of the following:
i) a volume of approximately 200 Å³,
ii) an electrostatic potential of −3.0 RTe_{c}⁻¹ or less,
iii) a plurality of hydrogen bond acceptors configured to form one or more hydrogen bonds in the presence of the amino acid ligand, and
iv) a plurality of van der Waals contact positions configured to form van der Waals interactions in the presence of the amino acid ligand.

51-79. (canceled)

80. A recombinant or synthetic amino acid binding protein having an amino acid sequence that is at least 80% identical to SEQ ID NO: 3, wherein the amino acid sequence comprises an amino acid substitution at one or more positions corresponding to 522, C23, Y24, C25, E26, 539, W75, D76, Y77, H78, C85, N120, H145, and M146 of SEQ ID NO: 3.

81-84. (canceled)

85. The amino acid binding protein of claim 80, wherein the amino acid sequence is at least 90% identical to a sequence selected from any one of PS1258-1260, PS1315-1318, PS1457-1478, PS1480-1499, PS1633-1656, PS1737-1758, PS1821-1898, PS2014-2057, and PS2116-2137 (SEQ ID NOs: 604-606, 660-663, 792-833, and 836-1025).

86. A recombinant or synthetic amino acid binding protein comprising a structure of Formula (III) or a structural equivalent thereof:
α1-α2-α3β1-β2-β3-β4-β5-α4-β6α5-α6 (III),
wherein:
each of α1, α2, α3, α4, α5, and α6 is an alpha-helix;
each of β1, β2, β3, β4, β5, and β6 is a beta-strand;
each instance of “-” is a loop; and
at least a portion of each of α2, β3, β4, α5, the loop between α1 and α2, and the loop between β3 and β4 form a binding pocket for an amino acid ligand, wherein the binding pocket comprises one or more of the following:
i) a volume of approximately 160 Å³,
ii) an electrostatic potential of −2.0 RTe_{c}⁻¹ or less,
iii) a plurality of hydrogen bond acceptors or donors configured to form one or more hydrogen bonds in the presence of the amino acid ligand,
iv) a plurality of van der Waals contact positions configured to form van der Waals interactions in the presence of the amino acid ligand, and
v) at least one negatively charged amino acid and at least one positively charged amino acid.

87-112. (canceled)

113. The amino acid binding protein of claim 86, comprising a structure of Formula (III-A) or a structural equivalent thereof:
α1-α2-α3β1-β2-β3-β4-β5-α4-β6α5-α6-α7-β7α8 (III-A),
wherein:
each of α7 and α8 is an alpha-helix; and
β7 is a beta-strand.

114. A recombinant or synthetic amino acid binding protein comprising a structure of Formula (III-B) or a structural equivalent thereof:
α1-α2-α3β1-β2-β3-β4 (III-B),
wherein:
each of α1, α2, and α3 is an alpha-helix;
each of β1, β2, β3, and β4 is a beta-strand;
each instance of “-” is a loop; and
at least a portion of each of α2, β3, β4, the loop between α1 and α2, and the loop between β3 and β4 form a binding pocket for an amino acid ligand, wherein the binding pocket comprises:
i) at least one negatively charged amino acid configured to form a hydrogen bond with the amino acid ligand, and
ii) at least one positively charged amino acid configured to form a hydrogen bond with the amino acid ligand.

115-187. (canceled)

188. A recombinant or synthetic amino acid binding protein having an amino acid sequence that is at least 80% identical to a sequence selected from any one of PS1165-1166 (SEQ ID NOs: 511-512), PS1267 (SEQ ID NO: 613), and PS1399-1424 (SEQ ID NOs: 742-767), wherein the amino acid binding protein comprises a luminescent label.

189. The amino acid binding protein of claim 188, wherein the amino acid sequence is at least 90% identical to PS1165 (SEQ ID NO: 511).

190. An amino acid recognizer comprising a polypeptide having at least a first amino acid binding protein and a second amino acid binding protein joined end-to-end, wherein the first and second amino acid binding proteins are separated by a linker comprising at least two amino acids, wherein at least one of the first and second amino acid binding proteins is an amino acid binding protein according to claim 188.


SEQUENCE LISTING (1147 sequences)

SEQ ID NO: 1 - protein, 119 aa, Homo sapiens
MPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEE

SEQ ID NO: 2 - protein, 83 aa, Homo sapiens
MHSKFSHAGRICGAKFKVGEPIYRCKECSFDDTCVLCVNCFNPKDHTGHHVYTTICTEFNNGICDCGDKEAWNHTLFCKAEEG

SEQ ID NO: 3 - protein, 200 aa, Homo sapiens
MNGLSAQHERIAPARHECVYTSCYCEENVWKLCEHIKTSKRCPLGDVYAVFISNERKMVPIWKQKSGRGEEPVIWDYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 4 - protein, 103 aa, synthetic construct
MSDSPVDLKPKPKVKPKLERPKLYKVMLLNDDYTTAFFVTKVLKAVFRMSEDTGRRVMMTAHRFGSAVVVVCERDIAETKAKEATDLGKEAGFPLMFTTEPEE

SEQ ID NO: 5 - protein, 103 aa, synthetic construct
MSDSPVDLKPKPKVKPKLERPKLYKVMLLNDDYTTMRFVTLVLKAVFRMSEDTGRRVMMTAHRFGSAVVVVCERDIAETKAKEATDLGKEAGFPLMFTTEPEE

SEQ ID NO: 6 - protein, 233 aa, synthetic construct
MLSATRRALQLFHSLFPIPRMGDSAAKIVSPQEALPGRKEPLVVAAKHHVNGNRTVEPFPEGTQMAVFGMGCFWGAERKFWTLKGVYSTQVGFAGGYTPNPTYKEVCSGKTGHAEVVRVVFQPEHISFEELLKVFWENHDPTQGMRQGNDHGSQYRSAIYPTSAEHVGAALKSKEDYQKVLSEHGFGLITTDIREGQTFYYAEDYHQQYLSKDPDGYCGLGGTGVSCPLGIKK

SEQ ID NO: 7 - protein, 233 aa, synthetic construct
MLSATRRALQLFHSLFPIPRMGDSAAKIVSPQEALPGRKEPLVVAAKHHVNGNRTVEPFPEGTQMAVFGMGSFWGAERKFWTLKGVYSTQVGFAGGYTPNPTYKEVCSGKTGHAEVVRVVFQPEHISFEELLKVFWENHDPTQGMRQGNDHGSQYRSAIYPTSAEHVGAALKSKEDYQKVLSEHGFGLITTDIREGQTFYYAEDYHQQYLSKDPDGYCGLGGTGVSCPLGIKK

SEQ ID NO: 8 - protein, 106 aa, synthetic construct
MAFPARGKTAPKNEVRRQPPYNVILLNDDDTTYRYVIEMLQKIFGFPPEKGFQIAEEVDRTGRVILLTTSKEHAELKQDQVHSYGPDPYLGRPCSGSMTCVIEPAV

SEQ ID NO: 9 - protein, 106 aa, synthetic construct
MAFPARGKTAPKNEVRRQPPYNVILLDDDDHTYRYVIEMLQKIFGFPPEKGFQIAEEVDRTGRVILLTTSKEHAELKQDQVHSYGPDPYLGRPCSGSMTCVIEPAV

SEQ ID NO: 10 - protein, 106 aa, synthetic construct
MAFPARGKTAPKNEVRRQPPYNVILLKDDDHTYRYVIEMLQKIFGFPPEKGFQIAEEVDRTGRVILLTTSKEHAELKQDQVHSYGPDPYLGRPCSGSMTCVIEPAV

SEQ ID NO: 11 - protein, 106 aa, synthetic construct
MAFPARGKTAPKNEVRRQPPYNVILLNKDDHTYRYVIEMLQKIFGFPPEKGFQIAEEVDRTGRVILLTTSKEHAELKQDQVHSYGPDPYLGRPCSGSMTCVIEPAV

SEQ ID NO: 12 - protein, 106 aa, synthetic construct
MAFPARGKTAPKNEVRRQPPYNVILLNDDDHTYRYVIEMLQKIFGFPPEKGFQIAEEVHRTGRVILLTTSKEHAELKQDQVHSYGPDPYLGRPCSGSMTCVIEPAV

SEQ ID NO: 13 - protein, 106 aa, synthetic construct
MAFPARGKTAPKNEVRRQPPYNVILLNDDNHTYRYVIEMLQKIFGFPPEKGFQIAEEVDRTGRVILLTTSKEHAELKQDQVHSYGPDPYLGRPCSGSMTCVIEPAV

SEQ ID NO: 14 - protein, 83 aa, synthetic construct
MGSVHKHTGRNCGRKFKIGEPLYRCHECGCDDTCVLCIHCFNPKDHVKHHVCTDICTEFTSGICDCGDEEAWNSPLHCKAEEQ

SEQ ID NO: 15 - protein, 292 aa, synthetic construct
MTSLNIMGRKFILERAKRNDNIEEIYTSAYVSLPSSTDTRLPHFKAKEEDCDVYEEGTNLVGKNAKYTYRSLGRHLDFLRPGLRFGGSQSSKYTYYTVEVKIDTVNLPLYKDSRSLDPHVTGTFTIKNLTPVLDKVVTLFEGYVINYNQFPLCSLHWPAEETLDPYMAQRESDCSHWKRFGHFGSDNWSLTERNFGQYNHESAEFMNQRYIYLKWKERFLLDDEEQENQMLDDNHHLEGASFEGFYYVCLDQLTGSVEGYYYHPACELFQKLELVPTNCDALNTYSSGFEIA

SEQ ID NO: 16 - protein, 609 aa, synthetic construct
MGSSHHHHHHHHHHSSGLVPRGSHMQIFVKTLTGKTITLEVEPSDTIENVKAKIQDKEGIPPDQQRLIFAGKQLEDGRTLSDYNIQKESTLHLVLRLRGGMASVVEYKGLKAGYYCGYCESREGKTSCGMWAHSMTVQDYQDLIDRGWRRSGKYVYKPVMDQTCCPQYTIRCHPLQFQPSKSHKKVLKKMLKFLAKGEISKGNCEDEPMDSTVEDAVDGDFALINKLDIKCDLKTLSDLKGSIESEEKEKEKSIKKEGSKEFIHPQSIEEKLGSGEPSHPIKVHIGPKPGKGADLSKPPCRKAREMRKERQRLKRMQQASAAASEAQGQPVCLLPKAKSNQPKSLEDLIFQSLPENASHKLEVRLVPASFEDPEFNSSFNQSFSLYTKYQVAIHQEAPEICEKSEFTRFLCSSPLEAEHPADGPECGYGSFHQQYWLDGKIIAVGVLDILPYCVSSVYLYYDPDYSFLSLGVYSALREIAFTRQLHEKTSQLSYYYMGFYIHSCPKMRYKGQYRPSDLLCPETYVWVPIEQCLPSLDNSKYCRFNQDPEAEDEGRSKELDRLRVFHRRSAMPYGVYKNHQEDPSEEAGVLEYANLVGQKCSERMLLFRH

SEQ ID NO: 17 - protein, 109 aa, synthetic construct
MSEPMTLPAIPQPRLKERTQRQPPYNVILLNDDDKSYEYVAAMLQVLFGYPPEKGYQMAKEVDSTGRVILLTTTREHAELKQEQIHAFGPDPNQARNSGSMKAVIEPAV

SEQ ID NO: 18 - protein, 109 aa, synthetic construct
MSEPMTLPAIPQPRLKERTQRQPPYNVILLNDDDKSYEYVIAMLQVLFGYPPEKGYQMAKEVDSTGRVILLTTTREHAELKQEQIHAFGPDPNQARNSGSMKAVIEPAV

SEQ ID NO: 19 - protein, 109 aa, synthetic construct
MSEPMTLPAIPQPRLKERTQRQPPYNVIILNDDDKSFEYVAAMLQVLFGYPPEKGYQMAKEIDSTGRVIMLTTTREHAELKQEQIHAFGPDPNQARNSGSMKAVIEPAV

SEQ ID NO: 20 - protein, 109 aa, synthetic construct
MSEPMTLPAIPQPRLKERTQRQPPYNVIILNDDDKSFEYVAALLQVLFGYPPEKGYQMAKEIDSTGRVIMLTTTREHAELKQEQIHAFGPDPNQARNSGSMKAVIEPAV

SEQ ID NO: 21 - protein, 109 aa, synthetic construct
MSEPMTLPAIPQPRLKERTQRQPPYNVIVLNDDDKSFEYVAAMLQVLFGYPPEKGYQVAKEIDSTGRVITLTTTREHAELKQEQIHAFGPDPNQARNSGSMKAVIEPAV

SEQ ID NO: 22 - protein, 119 aa, synthetic construct
MPTAASATESAIEDTPAPARPEVDGRTKPKRQPRYHVVQWNDDRHTYQYTVVMFQSLFGHPPERGYRLAKESDTQGRIIVLTTTREHAELKRDQIHAFGYDRLLARDKGSYKASIEAEE

SEQ ID NO: 23 - protein, 143 aa, synthetic construct
HHHHHHHHHHDYDIPTTENLYFQGMPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLARSKGSMKASIEAEE

SEQ ID NO: 24 - protein, 119 aa, synthetic construct
MPTAASATESAIEDTPAPARPEVDGRTKPKRQPRYHVVQWNDDRHTYQYTVVMFQSLFGHPPERGYRLAKESDTQGRIIVLTTTREHAELKRDQIHAFGYDPLQSGDKGSYKASIEAEE

SEQ ID NO: 25 - protein, 119 aa, synthetic construct
MPTAASATESAIEDTPAPARPEVDGRTKPKRQPRYHVVAWNDDRHTYQYTVVMFQSLFGHPPERGYRLAKEQDTQGRIIVLTTTREHAELKRDQIHAFGYDPLQSGDKGSYKASIEAEE

SEQ ID NO: 26 - protein, 119 aa, synthetic construct; identical to SEQ ID NO: 25

SEQ ID NO: 27 - protein, 119 aa, synthetic construct
MPTAASATESAIEDTPAPARPEVDGRTKPKRQPRYHVVSWNDDKHTYQWTVVMFQSLFGHPPERGYRLAKERDTQGRIIVLTTTREHAELKRDQIHAFGYDPLQSGDKGSMKASIEAEE

SEQ ID NO: 28 - protein, 91 aa, synthetic construct
MKMYNIPTPTMAQVIMVDDPITTTEFVISALRDFFDKSLEEAKALTSSIHRDGEGVCGVYPYDIARHRAAWVRDKAKALEFPLKLLVEEIK

SEQ ID NO: 29 - protein, 91 aa, synthetic construct
MKMYNIPTPTMAQVIMVDDPINTYEFTISALRDFFDKSLEEAKALASSIDRDGEGVCGVYPYDIARHRAAWVRDKAKALEFPEKLLVEEIK

SEQ ID NO: 30 - protein, 91 aa, synthetic construct
MKMYNIPTPTMAQVIMVDDPINTKEFTISALRDFFDKSLEEAKALASSIDRDGEGVCGVYPYDIARHRAAWVRDKAKALEFPEKLLVEEIK

SEQ ID NO: 31 - protein, 91 aa, synthetic construct
MKMYNIPTPTMAQVIRVDDPSMTNEFGISALRDFFDKSLEEAKALASSIDRDGEGVCGVYPYDIARHRAAWVRDKAKALEFPSKLLVEEIK

SEQ ID NO: 32 - protein, 91 aa, synthetic construct
MKMYNIPTPTMAQVIRVDDPSMTYEFGISALRDFFDKSLEEAKALASSIDRDGEGVCGVYPYDIARHRAAWVRDKAKALEFPSKLLVEEIK

SEQ ID NO: 33 - protein, 90 aa, synthetic construct
MPQERQQVTRKHYPNYKVIFLNSDFYTFQHLVALMMKYIPNMTSDRAWEISNQIHYEGQAIVWVGPQEQAELYHEQFLRAGLTMAPLEPE

SEQ ID NO: 34 - protein, 97 aa, synthetic construct
MTSTLRARPARDTDLQHRPYPHYRIITLDDDVMTFQHMANSYVTFLPGMTRDQMWAMSQQDDGEGSMVVWTGPQEQAELYHVQLGNHGQTNIPLEPV

SEQ ID NO: 35 - protein, 97 aa, synthetic construct
MTSTLRARPARDTDLQHRPYPHYRIIVLDDDVMTFQHMANSFVTFLPGMTRDQMWAMSQQDEGEGSMVVWTGPQEQAELYHVQLGNHGQTNIPLEPV

SEQ ID NO: 36 - protein, 97 aa, synthetic construct
MTSTLRARPARDTDLQHRPYPHYRIIVLDDDVMTFQHLANSFVTFLPGMTRDQMWAMSQQDDGEGSMVVWTGPQEQAELYHVQLGNHGQTNIPLEPV

SEQ ID NO: 37 - protein, 97 aa, synthetic construct
MTSTLRARPARDTDLQHRPYPHYRIIVLDDDVMTFQHMANSFVTFLPGMTRDQMWAMSQQDDGEGSMVVWTGPQEQAELYHVQLGNHGQTNIPLEPV

SEQ ID NO: 38 - protein, 97 aa, synthetic construct
MTSTLRARPARDTDLQHRPYPHYRIILLDSDVITFQLTANAFVTFLPGMTRDQMWAKIQQSDGEGSCVVWTGPQEQAELYHVQLGNQGLTEIPLEPV

SEQ ID NO: 39 - protein, 128 aa, synthetic construct
MVSIGAATVACAEGRPIFSGYFDWLAAMPETVTVPRTRLRPKTERPKLHKVIEVNQDYIPWEFWVTFFKGEFHMSEDQAQRKMIAGDRRGVYVVAVFTRDVAETKATRFSDHGRAKGYPTQMTTEPEE

SEQ ID NO: 40 - protein, 95 aa, synthetic construct
MGQTVEKPRVEGPGTGLGGSWRVITRNNDHYTRDHWARTIARFIPGVSLERAHEWSKVIHTTGRKVVYTGHKEAAEHYWQQFKGSGLESMPLEQG

SEQ ID NO: 41 - protein, 126 aa, synthetic construct
MTLSVALGPDTQESTQTGTAVSTDTLTAPDIPWNLVDWNDPVNLMSYISYVFQSYFGYSETKANKLMMEQDKKGRSIVAHGSKEQVEQHAVALHGYGNWATVEKATGGNSGGGKSGGPGKGKGKRG

SEQ ID NO: 42 - protein, 99 aa, synthetic construct
MSGTVVESKPRNSTQLAPRWKVIYHDNPVTTFDFTTGMFRRVFAKPPGEARRMTREAHDTGSVLVDVLALEQAEFRRDQMHSLARAEGFPQTLTLEPAD

SEQ ID NO: 43 - protein, 99 aa, synthetic construct
MSGTVVESKPRNSTQLAPRWKVIYHDQPVTTFDFTTGLFRRVFAKPPGEARRMTREAHDTGSVLVDVLALEQAEFRRDQMHSLARAEGFPQTLTLEPAD

SEQ ID NO: 44 - protein, 103 aa, synthetic construct
MSDSPVDLKPKPKVKPKLERPSMYKVITVNDDYTPMEFTIDHLQKFFSYDVERATQLMLASDYQGKAICGVFTAEVAETKVAMMNKSARENEHPELCTLEKAE

SEQ ID NO: 45 - protein, 103 aa, synthetic construct
MSDSPVDLKPKPKVKPKLERPSMYKVITVNDDYTPMEFTIDHLQKFFSYDVERATQLMLASEYQGKAICGVFTAEVAETKVAMMNKSARENEHPELCTLEKAE

SEQ ID NO: 46 - protein, 83 aa, synthetic construct
MHSKFNHAGRICGAKHRVGEPMYRCKECSFDDTCTLCVNCFNPKDHVGHHVYTSICTEFKNGICDCGDKEAWNHELNCKGAED

SEQ ID NO: 47 - protein, 83 aa, synthetic construct
MHSKFNHAGRICGAKFRVGEPLYRCKECSFDDTCVLCVNCFNPKDHVGHHVYTSICTEFLNGICDCGDKEAWNHELNCKGAED

SEQ ID NO: 48 - protein, 83 aa, synthetic construct
MHSKFNHAGRICGAKFRVGEPLYKCKECSFDDTCVLCVNCFNPKDHVGHHVYTMICTEFLNGICDCGDKEAWNHELNCKGAED

SEQ ID NO: 49 - protein, 106 aa, synthetic construct
MAFPARGKTAPKNEVRRQPPYNVIMLNDDDHTWRYAMELFQKIFGFPPEKGFQIVEEMDRTGRVILLTTSKEHAELKQDQMHSYGPDPYLGRPCSGSMTCVIEPAV

SEQ ID NO: 50 - protein, 106 aa, synthetic construct
MAFPARGKTAPKNEVRRQPPYNVIILNDDDHTWRYLMEMFQKIFGFPPEKGFQIIEEIDRTGRAILLTTSKEHAELKQDQLHSYGPDPYLGRPCSGSMTVVIEPAV

SEQ ID NO: 51 - protein, 106 aa, synthetic construct
MAFPARGKTAPKNEVRRQPPYNVILLNDDDHTWRYIMEMFQKIFGFPPEKGFQITEEIDRTGRAILLTTSKEHAELKQDQTHSYGPDPYLGRPCSGSMTMVIEPAV

SEQ ID NO: 52 - protein, 106 aa, synthetic construct
MAFPARGKTAPKNEVRRQPPYNVIILNDDDMTWRYLMEAFQKIFGFPPEKGFQIIEEIDRTGRAILLTTSKEHAELKQDQMHSYGPDPYLGRPCSGSMTMVIEPAV

SEQ ID NO: 53 - protein, 99 aa, synthetic construct
MSGTVVESKPRNSTQLAPRWKVIMHDQPVITFDFTLGMFRRVFAKPPGEARRITREAHDTGSVLVDVLALEQAEFRRDQMHSLARAEGFPLTMTLEPAD

SEQ ID NO: 54 - protein, 106 aa, synthetic construct
MAFPARGKTAPKNEVRRQPPYNVIILNDDDHTYRYFIEMFQKIFGFPPEKGFQYTEEMDRTGRLILLTTSKEHAELKQDQLHSYGPDPYLGRPCSGSVTVVIEPAV

SEQ ID NO: 55 - protein, 106 aa, synthetic construct
MAFPARGKTAPKNEVRRQPPYNVIILNDDDHTYRYFLEMFQKIFGFPPEKGFQYAEEIDRTGRLILLTTSKEHAELKQDQMHSYGPDPYLGRPCSGSITCVIEPAV

SEQ ID NO: 56 - protein, 106 aa, synthetic construct
MAFPARGKTAPKNEVRRQPPYNVIILNDDDHTYRYFIEMFQKIFGFPPEKGFQIVEEIDRTGRYILLTTSKEHAELKQDQLHSYGPDPYLGRPCSGSITCVIEPAV

SEQ ID NO: 57 - protein, 106 aa, synthetic construct
MAFPARGKTAPKNEVRRQPPYNVIMLNDDDHTYRYFIELFQKIFGFPPEKGFQIIEEIDRTGRAILLTTSKEHAELKQDQIHSYGPDPYLGRPCSGSITCVIEPAV

SEQ ID NO: 58 - protein, 106 aa, synthetic construct
MAFPARGKTAPKNEVRRQPPYNVIILNDDDHTYRYFLEMFQKIFGFPPEKGFQYVEEIDRTGRIILLTTSKEHAELKQDQMHSYGPDPYLGRPCSGSITCVIEPAV

SEQ ID NO: 59 - protein, 119 aa, synthetic construct
MPTAASATESAIEDTPAPARPEVDGRTKPKRQPRYHVVIWNDDDHTYQYFVVMFQSLFGHPPERGYRIVKEIDTQGRYIVLTTTREHAELKRDQLHAFGYDRLLARSKGSIKASIEAEE

SEQ ID NO: 60 - protein, 119 aa, synthetic construct
MPTAASATESAIEDTPAPARPEVDGRTKPKRQPRYHVVYWNDDDHTYQYFVVLFQSLFGHPPERGYRIVKEIDTQGRYIVLTTTREHAELKRDQTHAFGYDRLLARSKGSIKISIEAEE

SEQ ID NO: 61 - protein, 119 aa, synthetic construct
MPTAASATESAIEDTPAPARPEVDGRTKPKRQPRYHVVMWNDDDHTYQYFVVLLQSLFGHPPERGYRIVKEIDTQGRYIVLTTTREHAELKRDQIHAFGYDRLLARSKGSIKVSIEAEE

SEQ ID NO: 62 - protein, 119 aa, synthetic construct
MPTAASATESAIEDTPAPARPEVDGRTKPKRQPRYHVVFWNDDDHTYQYFVVLFQSLFGHPPERGYRIAKEIDTQGRYIVLTTTREHAELKRDQVHAFGYDRLLARSKGSIKISIEAEE

SEQ ID NO: 63 - protein, 119 aa, synthetic construct
MPTAASATESAIEDTPAPARPEVDGRTKPKRQPRYHVVIWNDDDHTYQYFVVTFQSLFGHPPERGYRIAKEIDTQGRYIVLTTTREHAELKRDQWHAFGYDRLLARSKGSIKCSIEAEE

SEQ ID NO: 64 - protein, 83 aa, synthetic construct
MHSKFSHAGRICGAKFKVGEPAYRCKECSFDDTCILCVNCFNPKDHTGHHVYTMICTEFLNGICDCGDKEAWNHTLFCKAEEG

SEQ ID NO: 65 - protein, 83 aa, synthetic construct
MHSKFSHAGRICGAKFKVGEPAYLCKECSFDDTCILCVNCFNPKDHTGHHVYTMICTEFLNGICDCGDKEAWNHTLFCKAEEG

SEQ ID NO: 66 - protein, 103 aa, synthetic construct
MSDSPVDLKPKPKVKPKLERPKLYKVMFLNDDYTPMSYIIVFFKAVFRMSEDTGRRKMMTAHRFGSMVVVVCERDIAETKAKEFTDHGKEAGFPIMMTTEPEE

SEQ ID NO: 67 - protein, 103 aa, synthetic construct
MSDSPVDLKPKPKVKPKLERPKLYKVMFLDDDYTPMSYIIVFFKAVFRMSEDTGRRKMMTAHRFGSMVVVVCERDIAETKAKEFTDHGKEAGFPIQMTTEPEE

SEQ ID NO: 68 - protein, 117 aa, synthetic construct
MIAEPICMQGEGDGEDGGTNRGTSVITRVKPKTKRPNLYRVLTLDDDYTPMEFMIHMFERFFQKDREAATRLMLLVHQHGVAECGVFTYEVAETKVSQMMDWARQHQHPFQMVMEKK

SEQ ID NO: 69 - protein, 90 aa, synthetic construct
MPQERQQVTRKHYPNYKVILLDMDFMTFAFMSAVLMKYIPNMTSDRSTELIRQAHYEGQTIVWVGPQEQAELYHEQFLRSGLQNMPLEPE

SEQ ID NO: 70 - protein, 97 aa, synthetic construct
MASAPSTTLDKSTQVVKKTYPNYKVIFLDSDLLTMDFLANVMIKYIPDMTTDRAWEKAYQMHYQGQFIVWTGPQEQAELYHQQFRREGLENIPLEAA

SEQ ID NO: 71 - protein, 97 aa, synthetic construct
MTSTLRARPARDTDLQHRPYPHYRIITLDNDVNTFQKIANVHVTFLPGMTRDQMWAKMQQVDGEGSVVVWTGPQEQAELYHVQFGNQGLKNIPLEPV

SEQ ID NO: 72 - protein, 96 aa, synthetic construct
MATETIERPRTRDPGSGLGGHWLVIMLDNDHMTFDLISKVLARVIPGVTVDDAYRFTYQMHQRGQVIIWRGPKEPAEHYWEQLQDVGLDNAPLERH

SEQ ID NO: 73 - protein, 106 aa, synthetic construct
MAFPARGKTAPKNEVRRQPPYNVIILNSDDHTYRYFMEMFQKIFGFPPEKGFQYMEEIDRTGRIILLTTSKEHAELKQDQSHSYGPDPYLGRPCSGSITMVIEPAV

SEQ ID NO: 74 - protein, 95 aa, synthetic construct
MGQTVEKPRVEGPGTGLGGSWRVISRDNDHYTFDEWVRIIARFIPGVSLERAHEWMKVLHTTGRMVVYTGHKEAAEHYWQQLKGAGFQSVPLEQG

SEQ ID NO: 75 - protein, 128 aa, synthetic construct
MVSIGAATVACAEGRPIFSGYFDWLAAMPETVTVPRTRLRPKTERPKLYKVIFVDDDFVPFEFIIRMFKAEFRMSEDQAAEKMMRAHQRGVQVVAVFTRDVAETKATRFTDWGRAKGYPLIMTTEPEE

SEQ ID NO: 76 - protein, 128 aa, synthetic construct
MVSIGAATVACAEGRPIFSGYFDWLAAMPETVTVPRTRLRPKTERPKLHKVIFVDQDYIPFEFIITMFKGEFHMSEDQAQRKLITAHRRGVYVVAVFTRDVAETKATRFSDAGRAKGYPLQVTTEPEE

SEQ ID NO: 77 - protein, 126 aa, synthetic construct
MTLSVALGPDTQESTQTGTAVSTDTLTAPDIPWNLVFWDDPVTLMSRIIYFFQSYFGYSETKAYKIVMEAHKKGRSIVAHGSKEQVEQHAVAFHGLGLWTTVEKATGGNSGGGKSGGPGKGKGKRG

SEQ ID NO: 78 - protein, 110 aa, synthetic construct
MSDTITLPGRPEVERDERTRRQPPYNVITHDKDDITFAYFIVMYNQLFGYPPEKGYEKLKEIHLNGRAIVLTTSKEHAELKRDQMHAWGPDPFSSKDCKGSISASIEPAY

SEQ ID NO: 79 - protein, 119 aa, synthetic construct
MPTAASATESAIEDTPAPARPEVDGRTKPKRQPRYHVVIWNDDDWTYQYYVVMFQSLFGHPPERGYRLMKELDTQGRFIVLTTTREHAELKRDQIHAFGYDRLLARSKGSIKASIEAEE

SEQ ID NO: 80 - protein, 116 aa, synthetic construct
MSSPSSLDDVQVSTSRAKPANETRTRKQPPYAVIFEDQDHVTHLWFYEMFMKVCGHAPEKGFVKSQQIHTQGKVMVWSGTLELAELKRDQFRGFGPDNYAPAPVTFPPGMTIEPLP

SEQ ID NO: 81 - protein, 99 aa, synthetic construct
MSGTVVESKPRNSTQLAPRWKVIFHDNPVTTFAFIIGMFRRVFAKPPGEAREMLRRAHDTGSVLVDVLALEQAEFRRDQFHSEARAEGFPSTMTLEPAD

SEQ ID NO: 82 - protein, 107 aa, synthetic construct
MHHHHHHHHHHDYDIPTTENLYFQGMHSKFSHAGRICGAKFKVGEPIYRCKECSFDDTCVLCVNCFNPKDHTGHHVYTTICTEFNNGICDCGDKEAWNHTLFCKAEE

SEQ ID NO: 83 - protein, 103 aa, synthetic construct
MSDSPVDLKPKPKVKPKLERPKLYKVMFLNQDYVPMSFIVVMFKAVFRMSEDTGRKKMMHAHRFGSVVVVVCERDIAETKAKEFTDYGKEAGFPVMMTTEPEE

SEQ ID NO: 84 - protein, 126 aa, synthetic construct
MTLSVALGPDTQESTQTGTAVSTDTLTAPDIPWNLVMWNQPVLLWSYMVYLFQSYFGYSETKTNKMVMEAHKKGRSIVAHGSKEQVEQHAVAMHGRGLWATVEKATGGNSGGGKSGGPGKGKGKRG

SEQ ID NO: 85 - protein, 110 aa, synthetic construct
MSDTITLPGRPEVERDERTRRQPPYNVITHNQDDITWEYFRVMYNQLFGYPPEKGYEKLKEIHLNGRIIVLTTSKEHAELKRDQMHAWGPDPFSSKDCKGSVSNSIEPAY

SEQ ID NO: 86 - protein, 110 aa, synthetic construct
MSDTITLPGRPEVERDERTRRQPPYNVIIHNTDDLTWEYFKVMFNQLFGYPPEKGYEKLKEIHLNGRAIVLTTSKEHAELKRDQMHAWGPDPFSSKDCKGSVSASIEPAY

SEQ ID NO: 87 - protein, 119 aa, synthetic construct
MPTAASATESAIEDTPAPARPEVDGRTKPKRQPRYHVVTWNTDDWTHQYYVVMYQSLFGHPPERGYRLTKEMDTQGRCIVLTTTREHAELKRDQMHAFGYDRLLARSKGSTKVSIEAEE

SEQ ID NO: 88 - protein, 119 aa, synthetic construct
MPTAASATESAIEDTPAPARPEVDGRTKPKRQPRYHVVMWNTDDWTYQYIIVMMQSLFGHPPERGYRMVKEMDTQGRTIVLTTTREHAELKRDQMHAFGYDRLLARSKGSIKNSIEAEE

SEQ ID NO: 89 - protein, 116 aa, synthetic construct
MSSPSSLDDVQVSTSRAKPANETRTRKQPPYAVIFENQDHVTILWFWEMFMKVCGHAPEKGFVKSQQIHTQGKVMVWSGTLELAELKRDQFRGFGPDNYAPRPVTFPPGMTIEPLP

SEQ ID NO: 90 - protein, 116 aa, synthetic construct
MSSPSSLDDVQVSTSRAKPANETRTRKQPPYAVIMENLDHITLLWMWEMFMKVCGHAPEKGFVKSQQNHTQGKVMVWSGTLELAELKRDQMRGWGPDNYAPRPVTFPPGFTIEPLP

SEQ ID NO: 91 - protein, 99 aa, synthetic construct
MSGTVVESKPRNSTQLAPRWKVIYHDQPVTTFDFIIGMFRRVFAKPPGEAREMTRRAHDTGSVLVDVLALEQAEFRRDQFHSEARAEGFPSTMTLEPAD

SEQ ID NO: 92 - protein, 99 aa, synthetic construct
MSGTVVESKPRNSTQLAPRWKVIYHDQPVMTFDFIIGLFRRVFAKPPGEARTITRIAHDTGSVLVDVLALEQAEFRRDQFHSEARAEGFPATMTLEPAD

SEQ ID NO: 93 - protein, 103 aa, synthetic construct
MSDSPVDLKPKPKVKPKLERPKLYKVMFLNQDYTPMSFIVVMFKAVFRMSEDTGRKKMMHAHRFGSVVVVVCERDIAETKAKEFTDYGKEAGFPSMMTTEPEE

SEQ ID NO: 94 - protein, 117 aa, synthetic construct
MIAEPICMQGEGDGEDGGTNRGTSVITRVKPKTKRPNLYRVLWLNHDYIPMEFMVHMFERFFQKDREAATRYMLLVHQHGVAECGVFTYEVAETKVSQLMDWARQHQHPFQVVMEKK

SEQ ID NO: 95 - protein, 117 aa, synthetic construct
MIAEPICMQGEGDGEDGGTNRGTSVITRVKPKTKRPNLYRVLWLNHDYIPMEFMVHMFERFFQKDREAATRIMLEVHQHGVSECGVFTYEVAETKVSQLMDFARQHQHPFQVVMEKK

SEQ ID NO: 96 - protein, 90 aa, synthetic construct
MPQERQQVTRKHYPNYKVIMLNNDFHTFQFMSAVMMKYIPNMTSDRSWEKVNQVHYEGQTIVWVGPQEQAELYHEQFLRSGLTNMPLEPE

SEQ ID NO: 97 - protein, 90 aa, synthetic construct
MPQERQQVTRKHYPNYKVIMLNDDFWTFQFLAAVIMKYIPNMTSDRVWEITNQVHYEGQSIVWVGPQEQAELYHEQFLREGFLHVPLEPE

SEQ ID NO: 98 - protein, 97 aa, synthetic construct
MASAPSTTLDKSTQVVKKTYPNYKVILLNNDLITRDKLANVLIKYIPDMTTDRAWERINQMHYQGQFIVWTGPQEQAELYHQQFRREGMQNIPLEAA

SEQ ID NO: 99 - protein, 97 aa, synthetic construct
MASAPSTTLDKSTQVVKKTYPNYKVIMLNNDLLTRDEIANVFIKYIPDMTTDRMWEMTNQMHYQGQLIVWTGPQEQAELYHQQFRREGLLNVPLEAA

SEQ ID NO: 100 - protein, 97 aa, synthetic construct
MTSTLRARPARDTDLQHRPYPHYRIITLDNDVITFQELVNYYVTFLPGMTRDQIWAKMQQVDGEGSAVVWTGPQEQAELYHVQLGNQGLFNCPLEPV

SEQ ID NO: 101 - protein, 97 aa, synthetic construct
MTSTLRARPARDTDLQHRPYPHYRIITLDMDVNTFQEIANYYVTFLPGMTRDQMWAWMQQVDGEGSVVVWTGPQEQAELYHVQLGNQGLYNIPLEPV

SEQ ID NO: 102 - protein, 96 aa, synthetic construct
MATETIERPRTRDPGSGLGGHWLVIHLNSDHFTFDEHAKWLARVIPGVTVDDAYRFTDQMHQRGQMIVWRGPKEPAEHYWEQLQDVGLSQSPLERH

SEQ ID NO: 103 - protein, 96 aa, synthetic construct
MATETIERPRTRDPGSGLGGHWLVIMLNSDHFTFDEFSKWLARVIPGVTVDDAYRFTDQMHQRGQVIVWRGPKEPAEHYWEQFQDIGLSQVPLERH

SEQ ID NO: 104 - protein, 106 aa, synthetic construct
MAFPARGKTAPKNEVRRQPPYNVIILNSDDHTYRYYMEMFQKIFGFPPEKGFQYMEEIDRTGRIILLTTSKEHAELKQDQLHSYGPDPYLGRPCSGSITCVIEPAV

SEQ ID NO: 105 - protein, 106 aa, synthetic construct
MAFPARGKTAPKNEVRRQPPYNVIILNSDDHTYRYFMEMFQKIFGFPPEKGFQYMEEIDRTGRIILLTTSKEHAELKQDQLHSYGPDPYLGRPCSGSITCVIEPAV

SEQ ID NO: 106 - protein, 95 aa, synthetic construct
MGQTVEKPRVEGPGTGLGGSWRVIMRNTDHITKDEFARSIARFIPGVSLERAHEKIKVMHTTGRFVVYTGHKEAAEHYWQQFKGSGVQVMPLEQG

SEQ ID NO: 107 - protein, 95 aa, synthetic construct
MGQTVEKPRVEGPGTGLGGSWRVIMRNDDHHTKDKFARMIARFIPGVSLERAHEKIKVLHTTGRMVVYTGHKEAAEHYWQQMKGAGVQNVPLEQG

SEQ ID NO: 108 - protein, 128 aa, synthetic construct
MVSIGAATVACAEGRPIFSGYFDWLAAMPETVTVPRTRLRPKTERPKLYKVIFVNRDFIPMEFIIRMFKAEFRMSEDQAARKMMYAHQRGVYVVAVFTRDVAETKATRFTDWGRAKGYPLLMTTEPEE

SEQ ID NO: 109 - protein, 128 aa, synthetic construct
MVSIGAATVACAEGRPIFSGYFDWLAAMPETVTVPRTRLRPKTERPKLYKVIFVNRDFIPMEFIIRMFKAEFRMSEDQAATKMMLAHQRGVQVVAVFTRDVAETKATRFTDWGRAKGYPLLMTTEPEE

SEQ ID NO: 110 - protein, 128 aa, synthetic construct
MVSIGAATVACAEGRPIFSGYFDWLAAMPETVTVPRTRLRPKTERPKLHKVIFVNQDYIPWEFIVTLFKGEFHMSEDQAQRKMIIAHRRGVYVVAVFTRDVAETKATRTSDWGRAKGYPLQFTTEPEE

SEQ ID NO: 111 - protein, 128 aa, synthetic construct
MVSIGAATVACAEGRPIFSGYFDWLAAMPETVTVPRTRLRPKTERPKLHKVIFVNKDYIPWEFIVTMFKGEFHMSEDQAQRKMIIAHRRGVYVVAVFTRDVAETKATRFSDWGRAKGYPLQMTTEPEE

SEQ ID NO: 112 - protein, 128 aa, synthetic construct
MVSIGAATVACAEGRPIFSGYFDWLAAMPETVTVPRTRLRPKTERPPLHKVILVNRDFIPMEFIIRMLKAEFRTTGDEAQRKMIYAHMKGSYVVAVFTREIAESKATRFTEWARAEGFPMLMTTEPEE

SEQ ID NO: 113 - protein, 128 aa, synthetic construct
MVSIGAATVACAEGRPIFSGYFDWLAAMPETVTVPRTRLRPKTERPPLHKVILVNQDFIPWEFMIRFLKAEFRTTGDEAQKKMISAHMKGSHVVAVFTREIAESKATRMTEWARAEGFPLLFTTEPEE

SEQ ID NO: 114 - protein, 126 aa, synthetic construct
MTLSVALGPDTQESTQTGTAVSTDTLTAPDIPWNLVIWNLPVLLWSFIVYLFQSYFGYSETKANKIVMEMHKKGRSIVAHGSKEQVEQHAVAFHGRGLWTTVEKATGGNSGGGKSGGPGKGKGKRG

SEQ ID NO: 115 - protein, 119 aa, synthetic construct
MPTAASATESAIEDTPAPARPEVDGRTKPKRQPRYHVVLWNDDDHTHQYVVVMLQSLFGHPPERGYRLAKEVDTQGRVIVLTTTREHAELKRDQIHAFGYDRLLARSKGSMKASIEAEE

SEQ ID NO: 116 - protein, 119 aa, synthetic construct
MPTAASATESAIEDTPAPARPEVDGRTKPKRQPRYHVVLWNDDDHTYQYVVMMLQSLFGHPPERGYRLAKEVDTQGRVIVLTTTREHAELKRDQIHAFGYDRLLARSKGSMKASIEAEE

SEQ ID NO: 117 - protein, 119 aa, synthetic construct
MPTAASATESAIEDTPAPARPEVDGRTKPKRQPRYHVVLWNDDDHTHQYVVMMLQSLFGHPPERGYRLAKEVDTQGRVIVLTTTREHAELKRDQIHAFGYDRLLARSKGSMKASIEAEE

SEQ ID NO: 118 - protein, 119 aa, synthetic construct
MPTAASATESAIEDTPAPARPEVDGRTKPKRQPRYHVVLWDDDDHTYQYVVVMLQSLFGHPPERGYRLAKEVDTQGRVIVLTTTREHAELKRDQIHAFGYDRLLARSKGSMKASIEAEE

SEQ ID NO: 119 - protein, 119 aa, synthetic construct
MPTAASATESAIEDTPAPARPEVDGRTKPKRQPRYHVVLWDYDDHTYQYVVVMLQSLFGHPPERGYRLAKEVDTQGRVIVLTTTREHAELKRDQIHAFGYDRLLARSKGSMKASIEAEE

SEQ ID NO: 120 - protein, 119 aa, synthetic construct
MPTAASATESAIEDTPAPARPEVDGRTKPKRQPRYHVVLWDYDDHTYQYVVVMLQSLFGHPPERGYRLAKELDTQGRVIVLTTTREHAELKRDQIHAFGYDRLLARSKGSMKASIEAEE

SEQ ID NO: 121 - protein, 119 aa, synthetic construct
MPTAASATESAIEDTPAPARPEVDGRTKPKRQPRYHVVLWNDEDHTYQYVVVMLQSLFGHPPERGYRLAKEVDTQGRVIVLTTTREHAELKRDQIHAFGYDRLLARSKGSMKASIEAEE

SEQ ID NO: 122 - protein, 119 aa, synthetic construct
MPTAASATESAIEDTPAPARPEVDGRTKPKRQPRYHVVLWNYDDHTYQYVVVMLQSLFGHPPERGYRLAKEVDTQGRVIVLTTTREHAELKRDQIHAFGYDRLLARSKGSMKASIEAEE

SEQ ID NO: 123 - protein, 126 aa, synthetic construct
MTLSVALGPDTQESTQTGTAVSTDTLTAPDIPWNLVIWDDPVNLMSYVSYVFQSYFGYSETKANKLMMEVHKKGRSIVAHGSKEQVEQHAVAMHGYGLWATVEKATGGNSGGGKSGGPGKGKGKRG

SEQ ID NO: 124 - protein, 110 aa, synthetic construct
MSDTITLPGRPEVERDERTRRQPPYNVILHDDDDHTFEYVIVMLNQLFGYPPEKGYEMAKEVHLNGRVIVLTTSKEHAELKRDQIHAFGPDPFSSKDCKGSMSASIEPAY

SEQ ID NO: 125 - protein, 119 aa, synthetic construct; identical to SEQ ID NO: 118

SEQ ID NO: 126 - protein, 116 aa, synthetic construct
MSSPSSLDDVQVSTSRAKPANETRTRKQPPYAVIVEDDDHHTFLYVIEALMKVCGHAPEKGFVLAQQIHTQGKAMVWSGTLELAELKRDQLRGFGPDNYAPRPVTFPLGVTIEPLP

SEQ ID NO: 127 - protein, 99 aa, synthetic construct
MSGTVVESKPRNSTQLAPRWKVIVHDDPVTTFDFVLGVLRRVFAKPPGEARRITREAHDTGSALVDVLALEQAEFRRDQAHSLARAEGFPLTLTLEPAD

SEQ ID NO: 128 - protein, 103 aa, synthetic construct
MSDSPVDLKPKPKVKPKLERPKLYKVMLLDDDYTPMSFVTVVLKAVFRMSEDTGRRVMMTAHRFGSAVVVVCERDIAETKAKEATDLGKEAGFPLMFTTEPEE

SEQ ID NO: 129 - protein, 117 aa, synthetic construct
MIAEPICMQGEGDGEDGGTNRGTSVITRVKPKTKRPNLYRVLLLDDDYTPMEFVIHILERFFQKDREAATRIMLHVHQHGVGECGVFTYEVAETKVSQVMDFARQHQHPLQCVMEKK

SEQ ID NO: 130 - protein, 90 aa, synthetic construct
MPQERQQVTRKHYPNYKVIVLDDDFNTFQHVAACLMKYIPNMTSDRAWELTNQVHYEGQAIVWVGPQEQAELYHEQLLRAGLTMAPLEPE

SEQ ID NO: 131 - protein, 97 aa, synthetic construct
MASAPSTTLDKSTQVVKKTYPNYKVIVLDDDLNTFDHVANCLIKYIPDMTTDRAWELTNQVHYQGQAIVWTGPQEQAELYHQQLRREGLTMAPLEAA

SEQ ID NO: 132 - protein, 96 aa, synthetic construct
MATETIERPRTRDPGSGLGGHWLVIVLDDDHNTFDHVAKTLARVIPGVTVDDGYRFADQIHQRGQAIVWRGPKEPAEHYWEQLQDAGLSMAPLERH

SEQ ID NO: 133 - protein, 106 aa, synthetic construct; identical to SEQ ID NO: 9

SEQ ID NO: 134 - protein, 95 aa, synthetic construct
MGQTVEKPRVEGPGTGLGGSWRVIVRDDDHNTFDHVARTLARFIPGVSLERGHEIAKVIHTTGRAVVYTGHKEAAEHYWQQLKGAGLTMAPLEQG

SEQ ID NO: 135 - protein, 128 aa, synthetic construct
MVSIGAATVACAEGRPIFSGYFDWLAAMPETVTVPRTRLRPKTERPKLYKVILVDDDFTPREFVVRVLKAEFRMSEDQAAKVMMTAHQRGVCVVAVFTRDVAETKATRATDAGRAKGYPLLFTTEPEE

SEQ ID NO: 136 - protein, 128 aa, synthetic construct
MVSIGAATVACAEGRPIFSGYFDWLAAMPETVTVPRTRLRPKTERPKLHKVILVDDDYTPREFVVTVLKGEFHMSEDQAQRVMITAHRRGVCVVAVFTRDVAETKATRASDAGRAKGYPLQFTTEPEE

SEQ ID NO: 137 - protein, 128 aa, synthetic construct
MVSIGAATVACAEGRPIFSGYFDWLAAMPETVTVPRTRLRPKTERPPLHKVILVDDDFTPREFVVRLLKAEFRTTGDEAQRIMITAHMKGSCVVAVFTREIAESKATRATETARAEGFPLLFTTEPEE

SEQ ID NO: 138 - protein, 126 aa, synthetic construct
MTLSVALGPDTQESTQTGTAVSTDTLTAPDIPWNLVIWDDPVNLMSYVSYVFQSYFGYSETKANKLMMEVDKKGRSIVAHGSKEQVEQHAVAMHGYGLWATVEKATGGNSGGGKSGGPGKGKGKRG

SEQ ID NO: 139 - protein, 110 aa, synthetic construct
MSDTITLPGRPEVERDERTRRQPPYNVILHDDDDHTFEYVIVMLNQLFGYPPEKGYEMAKEVDLNGRVIVLTTSKEHAELKRDQIHAFGPDPFSSKDCKGSMSASIEPAY

SEQ ID NO: 140 - protein, 116 aa, synthetic construct
MSSPSSLDDVQVSTSRAKPANETRTRKQPPYAVIVEDDDHHTFLYVIEALMKVCGHAPEKGFVLAQQIDTQGKAMVWSGTLELAELKRDQLRGFGPDNYAPRPVTFPLGVTIEPLP

SEQ ID NO: 141 - protein, 99 aa, synthetic construct
MSGTVVESKPRNSTQLAPRWKVIVHDDPVTTFDFVLGVLRRVFAKPPGEARRITREADDTGSALVDVLALEQAEFRRDQAHSLARAEGFPLTLTLEPAD

SEQ ID NO: 142 - protein, 103 aa, synthetic construct
MSDSPVDLKPKPKVKPKLERPKLYKVMLLDDDYTPMSFVTVVLKAVFRMSEDTGRRVMMTADRFGSAVVVVCERDIAETKAKEATDLGKEAGFPLMFTTEPEE

SEQ ID NO: 143 - protein, 117 aa, synthetic construct
MIAEPICMQGEGDGEDGGTNRGTSVITRVKPKTKRPNLYRVLLLDDDYTPMEFVIHILERFFQKDREAATRIMLHVDQHGVGECGVFTYEVAETKVSQVMDFARQHQHPLQCVMEKK

SEQ ID NO: 144 - protein, 90 aa, synthetic construct
MPQERQQVTRKHYPNYKVIVLDDDFNTFQHVAACLMKYIPNMTSDRAWELTNQVDYEGQAIVWVGPQEQAELYHEQLLRAGLTMAPLEPE

SEQ ID NO: 145 - protein, 97 aa, synthetic construct
MASAPSTTLDKSTQVVKKTYPNYKVIVLDDDLNTFDHVANCLIKYIPDMTTDRAWELTNQVDYQGQAIVWTGPQEQAELYHQQLRREGLTMAPLEAA

SEQ ID NO: 146 - protein, 96 aa, synthetic construct
MATETIERPRTRDPGSGLGGHWLVIVLDDDHNTFDHVAKTLARVIPGVTVDDGYRFADQIDQRGQAIVWRGPKEPAEHYWEQLQDAGLSMAPLERH

SEQ ID NO: 147 - protein, 95 aa, synthetic construct
MGQTVEKPRVEGPGTGLGGSWRVIVRDDDHNTFDHVARTLARFIPGVSLERGHEIAKVIDTTGRAVVYTGHKEAAEHYWQQLKGAGLTMAPLEQG

SEQ ID NO: 148 - protein, 128 aa, synthetic construct
MVSIGAATVACAEGRPIFSGYFDWLAAMPETVTVPRTRLRPKTERPKLYKVILVDDDFTPREFVVRVLKAEFRMSEDQAAKVMMTADQRGVCVVAVFTRDVAETKATRATDAGRAKGYPLLFTTEPEE

SEQ ID NO: 149 - protein, 128 aa, synthetic construct
MVSIGAATVACAEGRPIFSGYFDWLAAMPETVTVPRTRLRPKTERPKLHKVILVDDDYTPREFVVTVLKGEFHMSEDQAQRVMITADRRGVCVVAVFTRDVAETKATRASDAGRAKGYPLQFTTEPEE

SEQ ID NO: 150 - protein, 128 aa, synthetic construct
MVSIGAATVACAEGRPIFSGYFDWLAAMPETVTVPRTRLRPKTERPPLHKVILVDDDFTPREFVVRLLKAEFRTTGDEAQRIMITADMKGSCVVAVFTREIAESKATRATETARAEGFPLLFTTEPEE

SEQ ID NO: 151 - protein, 119 aa, synthetic construct
MPTAASATESAIEDTPAPARPEVDGRTKPKRQPRYHVVLWDDDDHTYQYVVVMLRSLFGHPPERGYRLAKEVDTQGRVIVLTTTREHAELKRDQIHAFGYDRLLARSKGSMKASIEAEE

SEQ ID NO: 152 - protein, 119 aa, synthetic construct
MPTAASATESAIEDTPAPARPEVDGRTKPKRQPRYHVVLWDDDDHTYQYVVVMLQSLFGHPPERGYRLAKEMDTQGRVIVLTTTREHAELKRDQIHAFGYDRLLARSKGSMKASIEAEE

SEQ ID NO: 153 - protein, 119 aa, synthetic construct
MPTAASATESAIEDTPAPARPEVDGRTKPKRQPRYHVVLWDDDDHTYQYVVVMLQSLFGHPPERGYRLAKEMDTLGRVIVLTTTREHAELKRDQIHAFGYDRLLARSKGSMKASIEAEE

SEQ ID NO: 154 - protein, 119 aa, synthetic construct
MPTAASATESAIEDTPAPARPEVDGRTKPKRQPRYHVVLWNDDDHTYQYVVVMLQSLFGHPPERGYRLAKEMDTLGRVIVLTTTREHAELKRDQIHAFGYDRLLARSKGSMKASIEAEE

SEQ ID NO: 155 - protein, 119 aa, synthetic construct
MPTAASATESAIEDTPAPARPEVDGRTKPKRQPRYHVVLWNDDYHTYQYVVVMLQSLFGHPPERGYRLAKEVDTQGRVIVLTTTREHAELKRDQIHAFGYDRLLARSKGSMKASIEAEE

SEQ ID NO: 156 - protein, 119 aa, synthetic construct
MPTAASATESAIEDTPAPARPEVDGRTKPKRQPRYHVVLWNDNYHTYQYVVVMLQSLFGHPPERGYRLAKEVDTQGRVIVLTTTREHAELKRDQIHAFGYDRLLARSKGSMKASIEAEE

SEQ ID NO: 157 - protein, 119 aa, synthetic construct
MPTAASATESAIEDTPAPARPEVDGRTKPKRQPRYHVVLWNDDDHTYQYVVVMLQSLFGHPPKRGYRLAKEVDTQGRVIVLTTTREHAELKRDQIHAFGYDRLLARSKGSMKASIEAEE

SEQ ID NO: 158 - protein, 119 aa, synthetic construct
MPTAASATESAIEDTPAPARPEVDGRTKPKRQPRYHVVLWNDDDHTYQYVVVMLQSLFGHSPERGYRLAKEVDTQGRVIVLTTTREHAELKRDQIHAFGYDRLLARSKGSMKASIEAEE

SEQ ID NO: 159 - protein, 119 aa, synthetic construct
MPTAASATESAIEDTPAPARPEVDGRTKPKRQPRYHVVLWNDDDHTYQYVVVMLQSLFGHPRERGYRLAKEVDTQGRVIVLTTTREHAELKRDQIHAFGYDRLLARSKGSMKASIEAEE

SEQ ID NO: 160 - protein, 119 aa, synthetic construct
MPTAASATESAIEDTPAPARPEVDGRTKPKRQPRYHVVLWNDADHTYQYVVVMLQSLFGHPPERGYRLAKEVDTQGRVIVLTTTREHAELKRDQIHAFGYDRLLARSKGSMKASIEAEE

SEQ ID NO: 161 - protein, 119 aa, synthetic construct
MPTAASATESAIEDTPAPARPEVDGRTKPKRQPRYHVVLWNDDDHTYQYVVVMLQSLFGHPPERGFRLAKEVDTQGRVIVLTTTREHAELKRDQIHAFGYDRLLARSKGSMKASIEAEE

SEQ ID NO: 162 - protein, 119 aa, synthetic construct
MPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLAHSKGSMKASIEAEE

SEQ ID NO: 163 - protein, 119 aa, synthetic construct
MPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDRLLAGSKGSMKASIEAEE

SEQ ID NO: 164 - protein, 119 aa, synthetic construct
MPTAASATESAIEDTPAPARPEVDGRTKPKRQPRYHVVLWNDDDHTYQYVVVMLRSLFGHPPERGYRLAKEVDTQGRVIVLTTTREHAELKRDQIHAFGYDRLLARSKGSMKASIEAEE

SEQ ID NO: 165 - protein, 119 aa, synthetic construct
MPTAASATESAIEDTPAPARPEVDGRTKPKRQPRYHVVLWNDDDHTYQYVVVMLQSLFGHPPERGYRLAKEMDTQGRVIVLTTTREHAELKRDQIHAFGYDRLLARSKGSMKASIEAEE

SEQ ID NO: 166 - protein, 119 aa, synthetic construct
MPTAASATESAIEDTPAPARPEVDSRTKPKRQPRYHVVNWNDDDLTCQYLVVMLQSLFGHPPERGYRLAKEVDTQGRVIVLTTTREHAELKRDQIHAFGYDRLLARSKGSIKASIEAEE

SEQ ID NO: 167 - protein, 119 aa, synthetic construct
MPTAASATESAIEDTPAPARPEVDSRTKPKRQPRYHVVNWNDDDLTCQYMVVMLQSLFGHPPERGYRLAKEVDTQGRVIVLTTTREHAELKRDQIHAFGYDRLLARSKGSTKASIEAEE

SEQ ID NO: 168 - protein, 119 aa, synthetic construct
MPTAASATESAIEDTPAPARPEVDGRTKPKRQPRYHVVNWNDDDPTRQYMVVMLQSLFGHPPERGYRLAKETDTQGRVIVLTTTREHAELKRDQIHAFGYDRLLARSKGSTKASIEAEE

SEQ ID NO: 169 - protein, 119 aa, synthetic construct
MPTAASATESAIEDTPAPARPEVDGRTKPKRQPRYHVVNWNDDDPTRQYLVVMLQSLFGHPPERGYRLAKETDTQGRVIVLTTTREHAELKRDQIHAFGYDRLLARSKGSTKASIEAEE

SEQ ID NO: 170 - protein, 119 aa, synthetic construct; identical to SEQ ID NO: 157

SEQ ID NO: 171 - protein, 119 aa, synthetic construct; identical to SEQ ID NO: 165

SEQ ID NO: 172 - protein, 119 aa, synthetic construct; identical to SEQ ID NO: 152

SEQ ID NO: 173 - protein, 119 aa, synthetic construct
MPTAASATESAIEDTPAPARPEVDGRTKPKRQPRYHVVLWDDDDHTYQYVVVMLQSLFGHPPKRGYRLAKEVDTQGRVIVLTTTREHAELKRDQIHAFGYDRLLARSKGSMKASIEAEE

SEQ ID NO: 174 - protein, 119 aa, synthetic construct
MPTAASATESAIEDTPAPARPEVDGRTKPKRQPRYHVVLWNDDDHTYQYVVVMLQSLFGHPPKRGYRLAKEMDTQGRVIVLTTTREHAELKRDQIHAFGYDRLLARSKGSMKASIEAEE

SEQ ID NO: 175 - protein, 119 aa, synthetic construct
MPTAASATESAIEDTPAPARPEVDGRTKPKRQPRYHVVLWDDDDHTYQYVVVMLQSLFGHPPKRGYRLAKEMDTQGRVIVLTTTREHAELKRDQIHAFGYDRLLARSKGSMKASIEAEE

SEQ ID NO: 176 - protein, 119 aa, synthetic construct
MPTAASGTESAIEDTPAPARPEVDGRTKPKRQPRYHVVLWDDDDHTYQYVVVMLRSLFGHPPERGYRLAKEVDTQGRVIVLTTTREHAELKRDQIHAFGYDRLLARSKGSMKASIEAEE

SEQ ID NO: 177 - protein, 119 aa, synthetic construct
MPTAASGTESAIEDTPAPARPEVDGRTKPKRQPRYHVVLWDDDDHTYQYVVVMLRSLFGHPPERGYRLAKEMDTQGRVIVLTTTREHAELKRDQIHAFGYDRLLARSKGSMKASIEAEE

SEQ ID NO: 178 - protein, 119 aa, synthetic construct
MPTAASGTESAIEDTPAPARPEVDGRTKPKRQPRYHVVLWDDDDHTYQYVVVMLRSLFGHPPKRGYRLAKEMDTQGRVIVLTTTREHAELKRDQIHAFGYDRLLARSKGSMKASIEAEE

SEQ ID NO: 179 - protein, 119 aa, synthetic construct
MPTAASATESAFEDTPAPARPEVDGRTKPKRQPRYHVVLWDDDDHTYQYVVVMLRSLFGHPPERGYRLAKEVDTQGRVIVLTTTREHAELKRDQIHAFGYDRLLARSKGSMKASIEAEE

SEQ ID NO: 180 - protein, 119 aa, synthetic construct
MPTAASATESAFEDTPAPARPEVDGRTKPKRQPRYHVVLWDDDDHTYQYVVVMLRSLFGHPPERGYRLAKEMDTQGRVIVLTTTREHAELKRDQIHAFGYDRLLARSKGSMKASIEAEE

SEQ ID NO: 181 - protein, 119 aa, synthetic construct
MPTAASATESAFEDTPAPARPEVDGRTKPKRQPRYHVVLWDDDDHTYQYVVVMLRSLFGHPPKRGYRLAKEVDTQGRVIVLTTTREHAELKRDQIHAFGYDRLLARSKGSMKASIEAEE

SEQ ID NO: 182 - protein, 119 aa, synthetic construct
MPTAASATESAFEDTPAPARPEVDGRTKPKRQPRYHVVLWDDDDHTYQYVVVMLRSLFGHPPKRGYRLAKEMDTQGRVIVLTTTREHAELKRDQIHAFGYDRLLARSKGSMKASIEAEE

SEQ ID NO: 183 - protein, 119 aa, synthetic construct
MPTAASATESAIEDTPAPARPEVDGRTKPKRQPRYHVVLWNDDDHTYQYVVVMLQSLFGHPRERGYRLAKEVDTLGRVIVLTTTREHAELKRDQIHAFGYDRLLARSKGSMKASIEAEE

SEQ ID NO: 184 - protein, 119 aa, synthetic construct
MPTAASATESAIEDTPAPARPEVDGRTKPKRQPRYHVVLWDDDDHTYQYVVVMLQSLFGHPRERGYRLAKEVDTLGRVIVLTTTREHAELKRDQIHAFGYDRLLARSKGSMKASIEAEE

SEQ ID NO: 185 - protein, 119 aa, synthetic construct
MPTAASATESAIEDTPAPARPEVDGRTKPKRQPRYHVVLWDDDDHTYQYVVVMLQSLFGHPRERGYRLAKEMDTLGRVIVLTTTREHAELKRDQIHAFGYDRLLARSKGSMKASIEAEE

SEQ ID NO: 186 - protein, 119 aa, synthetic construct
MPTAASATESAIEDTPAPARPEVDGRTKPKRQPRYHVVLWDDDDHTYQYVVVMLQSLFGHPRKRGYRLAKEMDTLGRVIVLTTTREHAELKRDQIHAFGYDRLLARSKGSMKASIEAEE

SEQ ID NO: 187 - protein, 119 aa, synthetic construct
MPTAASATESAIEDTPAPARPEVDGRTKPKRQPRYHVVLWNDDDHTYQYVVVMLQSLFGHPRKRGYRLAKEMDTLGRVIVLTTTREHAELKRDQIHAFGYDRLLARSKGSMKASIEAEE

SEQ ID NO: 188 - protein, 119 aa, synthetic construct
MPTAASATESAIEDTPAPARPVVDGRTKPKRQPRYHVVLWDDDDHTYQYVVVMLQSLFGHPPERGYRLAKEVDTQGRVIVLTTTREHAELKRDQIHAFGYDRLLARSKGSMKASIEAEE

SEQ ID NO: 189 - protein, 119 aa, synthetic construct
MPTAASATESAIEDTPAPARPVVDGRTKPKRQPRYHVVLWDDDDHTYQYVVVMLQSLFGHPPERGYRLAKEMDTQGRVIVLTTTREHAELKRDQIHAFGYDRLLARSKGSMKASIEAEE

SEQ ID NO: 190 - protein, 119 aa, synthetic construct
MPTAASATESAIEDTPAPARPVVDGRTKPKRQPRYHVVLWDDDDHTYQYVVVMLQSLFGHPPKRGYRLAKEMDTQGRVIVLTTTREHAELKRDQIHAFGYDRLLARSKGSMKASIEAEE

SEQ ID NO: 191 - protein, 119 aa, synthetic construct
MPTAASATESAIEDTPAPARPVVDGRTKPKRQPRYHVVLWDDDDHTYQYVVVMLQSLFGHPPKRGYRLAKEVDTQGRVIVLTTTREHAELKRDQIHAFGYDRLLARSKGSMKASIEAEE

SEQ ID NO: 192 - protein, 119 aa, synthetic construct; identical to SEQ ID NO: 159

SEQ ID NO: 193 - protein, 119 aa, synthetic construct
MPTAASATESAIEDTPAPARPEVDGRTKPKRQPRYHVVLWDDDDHTYQYVVVMLQSLFGHPRERGYRLAKEVDTQGRVIVLTTTREHAELKRDQIHAFGYDRLLARSKGSMKASIEAEE

SEQ ID NO: 194 - protein, 119 aa, synthetic construct
MPTAASATESAIEDTPAPARPEVDGRTKPKRQPRYHVVLWDDDDHTYQYVVVMLQSLFGHPRERGYRLAKEMDTQGRVIVLTTTREHAELKRDQIHAFGYDRLLARSKGSMKASIEAEE

SEQ ID NO: 195 - protein, 119 aa, synthetic construct
MPTAASATESAIEDTPAPARPEVDGRTKPKRQPRYHVVLWDDDDHTYQYVVVMLQSLFGHPRKRGYRLAKEMDTQGRVIVLTTTREHAELKRDQIHAFGYDRLLARSKGSMKASIEAEE

SEQ ID NO: 196 - protein, 120 aa, synthetic construct
MPTAASATESAIEDTPAPARPEVDGRTKPKRQPRYHVVLWNDDDHTYQYVVVMLQSLFGHPRKRGYRLAKEVMDTQGRVIVLTTTREHAELKRDQIHAFGYDRLLARSKGSMKASIEAEE

SEQ ID NO: 197 - protein, 119 aa, synthetic construct
MPTAASATESAIEDTPAPARPEVDGRTKPKRQPRYHVVLWNDDDHTYQYVVVMLQSLFGHSPKRGYRLAKEVDTQGRVIVLTTTREHAELKRDQIHAFGYDRLLARSKGSMKASIEAEE

SEQ ID NO: 198 - protein, 119 aa, synthetic construct
MPTAASATESAIEDTPAPARPEVDGRTKPKRQPRYHVVLWDDDDHTYQYVVVMLQSLFGHSPKRGYRLAKEVDTQGRVIVLTTTREHAELKRDQIHAFGYDRLLARSKGSMKASIEAEE

SEQ ID NO: 199 - protein, 119 aa, synthetic construct
MPTAASATESAIEDTPAPARPEVDGRTKPKRQPRYHVVLWDDDDHTYQYVVVMLQSLFGHSPKRGYRLAKEMDTQGRVIVLTTTREHAELKRDQIHAFGYDRLLARSKGSMKASIEAEE

SEQ ID NO: 200 - protein, 119 aa, synthetic construct
MPTAASATESAIEDTPAPARPEVDGRTKPKRQPRYHVVLWNDDDHTYQYVVVMLQSLFGHSPKRGYRLAKEMDTQGRVIVLTTTREHAELKRDQIHAFGYDRLLARSKGSMKASIEAEE

SEQ ID NO: 201 - protein, 119 aa, synthetic construct; identical to SEQ ID NO: 164

SEQ ID NO: 202 - protein, 119 aa, synthetic construct; identical to SEQ ID NO: 151

SEQ ID NO: 203 - protein, 119 aa, synthetic construct
MPTAASATESAIEDTPAPARPEVDGRTKPKRQPRYHVVLWDDDDHTYQYVVVMLRSLFGHPPERGYRLAKEMDTQGRVIVLTTTREHAELKRDQIHAFGYDRLLARSKGSMKASIEAEE

SEQ ID NO: 204 - protein, 119 aa, synthetic construct
MPTAASATESAIEDTPAPARPEVDGRTKPKRQPRYHVVLWDDDDHTYQYVVVMLRSLFGHPPKRGYRLAKEMDTQGRVIVLTTTREHAELKRDQIHAFGYDRLLARSKGSMKASIEAEE

SEQ ID NO: 205 - protein, 119 aa, synthetic construct
MPTAASATESAIEDTPAPARPEVDGRTKPKRQPRYHVVLWNDDDHTYQYVVVMLRSLFGHPPKRGYRLAKEMDTQGRVIVLTTTREHAELKRDQIHAFGYDRLLARSKGSMKASIEAEE

SEQ ID NO: 206 - protein, 119 aa, synthetic construct
MPTAASATESAIEDTPAPARPEVDGRTKPKHQPRYHVVLWNDDDHTYQYVVVMLRSLFGHPPERGYRLAKEVDTQGRVIVLTTTREHAELKRDQIHAFGYDRLLARSKGSMKASIEAEE

SEQ ID NO: 207 - protein, 119 aa, synthetic construct
MPTAASATESAIEDTPAPARPEVDGRTKPKHQPRYHVVLWDDDDHTYQYVVVMLRSLFGHPPERGYRLAKEVDTQGRVIVLTTTREHAELKRDQIHAFGYDRLLARSKGSMKASIEAEE

SEQ ID NO: 208 - protein, 119 aa, synthetic construct
MPTAASATESAIEDTPAPARPEVDGRTKPKHQPRYHVVLWDDDDHTYQYVVVMLRSLFGHPPERGYRLAKEMDTQGRVIVLTTTREHAELKRDQIHAFGYDRLLARSKGSMKASIEAEE

SEQ ID NO: 209 - protein, 119 aa, synthetic construct
MPTAASATESAIEDTPAPARPEVDGRTKPKHQPRYHVVLWDDDDHTYQYVVVMLRSLFGHPPKRGYRLAKEMDTQGRVIVLTTTREHAELKRDQIHAFGYDRLLARSKGSMKASIEAEE

SEQ ID NO: 210 - protein, 119 aa, synthetic construct
MPTAASATESAIEDTPAPARPEVDGRTKPKHQPRYHVVLWNDDDHTYQYVVVMLRSLFGHPPKRGYRLAKEMDTQGRVIVLTTTREHAELKRDQIHAFGYDRLLARSKGSMKASIEAEE

SEQ ID NO: 211 - protein, 119 aa, synthetic construct
MPTAASATESAIEDTPAPARPEVDGRTKPKRQPRYHVVLWNDDDHTYQYVVVMLQSLFGHPPERGYRMAKEVDTQGRVIVLTTTREHAELKRDQIHAFGYDRLLARSKGSMKASIEAEE

SEQ ID NO: 212 - protein, 119 aa, synthetic construct
MPTAASATESAIEDTPAPARPEVDGRTKPKRQPRYHVVLWDDDDHTYQYVVVMLQSLFGHPPERGYRMAKEVDTQGRVIVLTTTREHAELKRDQIHAFGYDRLLARSKGSMKASIEAEE

SEQ ID NO: 213 - protein, 119 aa, synthetic construct
MPTAASATESAIEDTPAPARPEVDGRTKPKRQPRYHVVLWDDDDHTYQYVVVMLQSLFGHPPERGYRMAKEMDTQGRVIVLTTTREHAELKRDQIHAFGYDRLLARSKGSMKASIEAEE

SEQ ID NO: 214 - protein, 119 aa, synthetic construct
MPTAASATESAIEDTPAPARPEVDGRTKPKRQPRYHVVLWDDDDHTYQYVVVMLQSLFGHPPKRGYRMAKEMDTQGRVIVLTTTREHAELKRDQIHAFGYDRLLARSKGSMKASIEAEE

SEQ ID NO: 215 - protein, 119 aa, synthetic construct
MPTAASATESAIEDTPAPARPEVDGRTKPKRQPRYHVVLWNDDDHTYQYVVVMLQSLFGHPPKRGYRMAKEMDTQGRVIVLTTTREHAELKRDQIHAFGYDRLLARSKGSMKASIEAEE

SEQ ID NO: 216 - protein, 119 aa, synthetic construct
MPTAASATESAIEDTPAPARPEVDGRTKPKRQPRYHVVLWNDDDHTYQYVVVMLQSLLGHPPERGYRLAKEMDTQGRVIVLTTTREHAELKRDQIHAFGYDRLLARSKGSMKASIEAEE

SEQ ID NO: 217 - protein, 119 aa, synthetic construct
MPTAASATESAIEDTPAPARPEVDGRTKPKRQPRYHVVLWDDDDHTYQYVVVMLQSLLGHPPERGYRLAKEMDTQGRVIVLTTTREHAELKRDQIHAFGYDRLLARSKGSMKASIEAEE

SEQ ID NO: 218 - protein, 119 aa, synthetic construct
MPTAASATESAIEDTPAPARPEVDGRTKPKRQPRYHVVLWDDDDHTYQYVVVMLQSLLGHPPKRGYRLAKEMDTQGRVIVLTTTREHAELKRDQIHAFGYDRLLARSKGSMKASIEAEE

SEQ ID NO: 219 - protein, 119 aa, synthetic construct
MPTAASATESAIEDTPAPARPEVDGRTKPKRQPRYHVVLWNDDDHTYQYVVVMLQSLLGHPPKRGYRLAKEMDTQGRVIVLTTTREHAELKRDQIHAFGYDRLLARSKGSMKASIEAEE

SEQ ID NO: 220 - protein, 119 aa, synthetic construct
MPTAASATESAIEDTPAPARPEVDGRTKPKRQPRYHVVLWNDDDHTYQYVVVMLQSLFGHPPGRGYRLAKEVDTQGRVIVLTTTREHAELKRDQIHAFGYDRLLARSKGSMKASIEAEE

SEQ ID NO: 221 - protein, 119 aa, synthetic construct
MPTAASATESAIEDTPAPARPEVDGRTKPKRQPRYHVVLWDDDDHTYQYVVVMLQSLFGHPPGRGYRLAKEVDTQGRVIVLTTTREHAELKRDQIHAFGYDRLLARSKGSMKASIEAEE

SEQ ID NO: 222 - protein, 119 aa, synthetic construct
MPTAASATESAIEDTPAPARPEVDGRTKPKRQPRYHVVLWDDDDHTYQYVVVMLQSLFGHPPGRGYRLAKEMDTQGRVIVLTTTREHAELKRDQIHAFGYDRLLARSKGSMKASIEAEE

SEQ ID NO: 223 - protein, 119 aa, synthetic construct
MPTAASATESAIEDTPAPARPEVDGRTKPKRQPRYHVVLWNDDDHTYQYVVVMLQSLFGHPPGRGYRLAKEMDTQGRVIVLTTTREHAELKRDQIHAFGYDRLLARSKGSMKASIEAEE

SEQ ID NO: 224 - protein, 119 aa, synthetic construct; identical to SEQ ID NO: 220

SEQ ID NO: 225 - protein, 119 aa, synthetic construct
MPTAASATESAIEDTPAPARPEVDGRTKPKRQPRYHVVLWDDDDHTYQYVVVMLQSLFGHPPGRGYRLAKEVDTQGRVIVLTTTREHAELKRDQIHAFGYDRLLAHSKGSMKASIEAEE

SEQ ID NO: 226 - protein, 119 aa, synthetic construct
MPTAASATESAIEDTPAPARPEVDGRTKPKRQPRYHVVLWDDDDHTYQYVVVMLQSLFGHPPGRGYRLAKEMDTQGRVIVLTTTREHAELKRDQIHAFGYDRLLAHSKGSMKASIEAEE

SEQ ID NO: 227 - protein, 107 aa, synthetic construct
MPSAAPAKPKTKRQSRTQGMPPYNVVLLDDDDHTYGYVIEMLRKVFGHPPEKGFELATEVDKNGRVIVMTTNLEVAELKRDEVHAFGPDPLMPRSKGSMSAVVERAG

SEQ ID NO: 228 - protein, 107 aa, synthetic construct
MPSAAPAKPKTKRQSRTQGMPPYNVVLLDDDDHTYGYVIEMLNKVFGHPPEKGFELATEMDKNGRVIVMTTNLEVAELKRDEVHAFGPDPLMPRSKGSMSAVVERAG

SEQ ID NO: 229 - protein, 107 aa, synthetic construct
MPSAAPAKPKTKRQSRTQGMPPYNVVLLDDDDHTYGYVIEMLRKVFGHPPEKGFELATEMDKNGRVIVMTTNLEVAELKRDEVHAFGPDPLMPRSKGSMSAVVERAG

SEQ ID NO: 230 - protein, 107 aa, synthetic construct
MPSAAPAKPKTKRQSRTQGMPPYNVVLLDDDDHTYGYVIEMLNKVFGHPREKGFELATEVDKLGRVIVMTTNLEVAELKRDEVHAFGPDPLMPRSKGSMSAVVERAG

SEQ ID NO: 231 - protein, 107 aa, synthetic construct
MPSAAPAKPKTKRQSRTQGMPPYNVVLLDDDDHTYGYVIEMLRKVFGHPREKGFELATEMDKLGRVIVMTTNLEVAELKRDEVHAFGPDPLMPRSKGSMSAVVERAG

SEQ ID NO: 232 - protein, 107 aa, synthetic construct
MPSAAPAKPVTKRQSRTQGMPPYNVVLLDDDDHTYGYVIEMLNKVFGHPPEKGFELATEVDKNGRVIVMTTNLEVAELKRDEVHAFGPDPLMPRSKGSMSAVVERAG

SEQ ID NO: 233 - protein, 107 aa, synthetic construct
MPSAAPAKPVTKRQSRTQGMPPYNVVLLDDDDHTYGYVIEMLRKVFGHPPEKGFELATEMDKNGRVIVMTTNLEVAELKRDEVHAFGPDPLMPRSKGSMSAVVERAG

SEQ ID NO: 234 - protein, 107 aa, synthetic construct
MPSAAPAKPKTKRQSRTQGMPPYNVVLLDDDDHTYGYVIEMLNKVFGHPREKGFELATEVDKNGRVIVMTTNLEVAELKRDEVHAFGPDPLMPRSKGSMSAVVERAG

SEQ ID NO: 235 - protein, 107 aa, synthetic construct
MPSAAPAKPKTKRQSRTQGMPPYNVVLLDDDDHTYGYVIEMLRKVFGHPREKGFELATEVDKNGRVIVMTTNLEVAELKRDEVHAFGPDPLMPRSKGSMSAVVERAG

SEQ ID NO: 236 - protein, 107 aa, synthetic construct
MPSAAPAKPKTKRQSRTQGMPPYNVVLLDDDDHTYGYVIEMLRKVFGHPREKGFELATEMDKNGRVIVMTTNLEVAELKRDEVHAFGPDPLMPRSKGSMSAVVERAG

SEQ ID NO: 237 - protein, 107 aa, synthetic construct
MPSAAPAKPKTKRQSRTQGMPPYNVVLLDDDDHTYGYVIEMLNKVFGHSPEKGFELATEVDKNGRVIVMTTNLEVAELKRDEVHAFGPDPLMPRSKGSMSAVVERAG

SEQ ID NO: 238 - protein, 107 aa, synthetic construct
MPSAAPAKPKTKRQSRTQGMPPYNVVLLDDDDHTYGYVIEMLRKVFGHSPEKGFELATEMDKNGRVIVMTTNLEVAELKRDEVHAFGPDPLMPRSKGSMSAVVERAG

SEQ ID NO: 239 - protein, 107 aa, synthetic construct
MPSAAPAKPKTKRQSRTQHMPPYNVVLLDDDDHTYGYVIEMLRKVFGHPPEKGFELATEVDKNGRVIVMTTNLEVAELKRDEVHAFGPDPLMPRSKGSMSAVVERAG

SEQ ID NO: 240 - protein, 107 aa, synthetic construct
MPSAAPAKPKTKRQSRTQHMPPYNVVLLDDDDHTYGYVIEMLRKVFGHPPEKGFELATEMDKNGRVIVMTTNLEVAELKRDEVHAFGPDPLMPRSKGSMSAVVERAG

SEQ ID NO: 241 - protein, 107 aa, synthetic construct
MPSAAPAKPKTKRQSRTQGMPPYNVVLLDDDDHTYGYVIEMLNKVFGHPPEKGFEMATEVDKNGRVIVMTTNLEVAELKRDEVHAFGPDPLMPRSKGSMSAVVERAG

SEQ ID NO: 242 - protein, 107 aa, synthetic construct
MPSAAPAKPKTKRQSRTQGMPPYNVVLLDDDDHTYGYVIEMLRKVFGHPPEKGFEMATEMDKNGRVIVMTTNLEVAELKRDEVHAFGPDPLMPRSKGSMSAVVERAG

SEQ ID NO: 243 - protein, 107 aa, synthetic construct
MPSAAPAKPKTKRQSRTQGMPPYNVVLLDDDDHTYGYVIEMLNKVLGHPPEKGFELATEMDKNGRVIVMTTNLEVAELKRDEVHAFGPDPLMPRSKGSMSAVVERAG

SEQ ID NO: 244 - protein, 107 aa, synthetic construct
MPSAAPAKPKTKRQSRTQGMPPYNVVLLDDDDHTYGYVIEMLRKVLGHPPEKGFELATEMDKNGRVIVMTTNLEVAELKRDEVHAFGPDPLMPRSKGSMSAVVERAG

SEQ ID NO: 245 - protein, 107 aa, synthetic construct
MPSAAPAKPKTKRQSRTQGMPPYNVVLLDDDDHTYGYVIEMLNKVFGHPPGKGFELATEVDKNGRVIVMTTNLEVAELKRDEVHAFGPDPLMPRSKGSMSAVVERAG

SEQ ID NO: 246 - protein, 107 aa, synthetic construct
MPSAAPAKPKTKRQSRTQGMPPYNVVLLDDDDHTYGYVIEMLRKVFGHPPGKGFELATEMDKNGRVIVMTTNLEVAELKRDEVHAFGPDPLMPRSKGSMSAVVERAG

SEQ ID NO: 247 - protein, 107 aa, synthetic construct
MPSAAPAKPKTKRQSRTQGMPPYNVVLLDDDDHTYGYVIEMLNKVFGHPPGKGFELATEVDKNGRVIVMTTNLEVAELKRDEVHAFGPDPLMPHSKGSMSAVVERAG

SEQ ID NO: 248 - protein, 107 aa, synthetic construct
MPSAAPAKPKTKRQSRTQGMPPYNVVLLDDDDHTYGYVIEMLRKVFGHPPGKGFELATEMDKNGRVIVMTTNLEVAELKRDEVHAFGPDPLMPHSKGSMSAVVERAG

SEQ ID NO: 249 - protein, 119 aa, synthetic construct
MPTAASATESAIEDTPAPARPEVDGRTKPKRQPRYHVVLWDDDDHTYQYVVVMLQSLFGHPPKRGYRLAKEMDTLGRVIVLTTTREHAELKRDQIHAFGYDRLLARSKGSMKASIEAEE

SEQ ID NO: 250 - protein, 119 aa, synthetic construct
MPTAASATESAIEDTPAPARPEVDGRTKPKRQPRYHVVNWNDDDPTRQYTVVMLQSLFGHPPERGYRLAKETRTQGRVIVLTTTREHAELKRDQIHAFGYDRLLARSKGSTKASIEAEE

SEQ ID NO: 251 - protein, 119 aa, synthetic construct
MPTAASATESAIEDTPAPARPEVDSRTKPKRQPRYHVVNWNDDDLTCQYTVVMLQSLFGHPPERGYRLAKEVDTQGRVIVLTTTREHAELKRDQIHAFGYDRLLARSKGSHKASIEAEE

SEQ ID NO: 252 - protein, 119 aa, synthetic construct
MPTAASATESAIEDTPAPARPEVDGRTKPKRQPRYHVVSWNDDDHTSQYTVVMLQSLFGHPPERGYRLAKELHTQGRVIVLTTTREHAELKRDQIHAFGYDRLLARSKGSPKASIEAEE

SEQ ID NO: 253 - protein, 119 aa, synthetic construct
MPTAASATESAIEDTPAPARPEVDGRTKPKRQPRYHVVLWNDDDHTYQYVVVMLQSLFGHPPKRGYRMAKEVDTQGRVIVLTTTREHAELKRDQIHAFGYDRLLARSKGSMKASIEAEE

SEQ ID NO: 254 - protein, 119 aa, synthetic construct
MPTAASATESAIEDTPAPARPEVDGRTKPKRQPRYHVVLWNDDDHTYQYVVVMLQSLFGHPRKRGYRLAKEVDTQGRVIVLTTTREHAELKRDQIHAFGYDRLLARSKGSMKASIEAEE

SEQ ID NO: 255 - protein, 119 aa, synthetic construct
MPTAASATESAIEDTPAPARPEVDGRTKPKRQPRYHVVLWNDDDHTYQYVVVMLQSLFGHPPGRGYRLAKEVDTQGRVIVLTTTREHAELKRDQIHAFGYDRLLAHSKGSMKASIEAEE

SEQ ID NO: 256 - protein, 119 aa, synthetic construct
MPTAASATESAIEDTPAPARPEVDGRTKPKRQPRYHVVLWNDDDHTYQYVVVMLQSLFGHPPKRGYRLAKEVDTQGRVIVLTTTREHAELKRDQIHAFGYDRLLAHSKGSMKASIEAEE

SEQ ID NO: 257 - protein, 119 aa, synthetic construct
MPTAASATESAIEDTPAPARPEVDGRTKPKRQPRYHVVLWNDDDHTYQYVVVMLQSLFGHPPRRGYRLAKEVDTQGRVIVLTTTREHAELKRDQIHAFGYDRLLARSKGSMKASIEAEE

SEQ ID NO: 258 - protein, 119 aa, synthetic construct
MPTAASATESAIEDTPAPARPEVDGRTKPKRQPRYHVVLWNDDDHTYQYVVVMLQSLFGHPPDRGYRLAKEVDTQGRVIVLTTTREHAELKRDQIHAFGYDRLLARSKGSMKASIEAEE

SEQ ID NO: 259 - protein, 119 aa, synthetic construct
MPTAASATESAIEDTPAPARPEVDGRTKPKRQPRYHVVLWDDDDHTYQYVVVMLQSLFGHPPKRGYRLAKEVDTQGRVIVLTTTREHAELKRDQIHAFGYDRLLAHSKGSMKASIEAEE

SEQ ID NO: 260 - protein, 119 aa, synthetic construct
MPTAASATESAIEDTPAPARPEVDGRTKPKRQPRYHVVLWDDDDHTYQYVVVMLQSLFGHPPKRGYRLAKEMDTQGRVIVLTTTREHAELKRDQIHAFGYDRLLAHSKGSMKASIEAEE

SEQ ID NO: 261 - protein, 119 aa, synthetic construct
MPTAASATESAIEDTPAPARPEVDGRTKPKRQPRYHVVLWNDDDHTYQYVVVMLRSLFGHPPERGYRLAKEVDTQGRVIVLTTTREHAELKRDQIHAFGYDRLLAHSKGSMKASIEAEE

SEQ ID NO: 262 - protein, 119 aa, synthetic construct
MPTAASATESAIEDTPAPARPEVDGRTKPKRQPRYHVVLWNDNYHTYQYVVVMLQSLFGHPPERGYRLAKEVDTQGRVIVLTTTREHAELKRDQIHAFGYDRLLAHSKGSMKASIEAEE

SEQ ID NO: 263 - protein, 119 aa, synthetic construct
MPTAASATESAIEDTPAPARPEVDGRTKPKRQPRYHVVLWNDNDHTYQYVVVMLQSLFGHPPERGYRLAKEVDTQGRVIVLTTTREHAELKRDQIHAFGYDRLLAHSKGSMKASIEAEE

SEQ ID NO: 264 - protein, 119 aa, synthetic construct
MPTAASATESAIEDTPAPARPEVDGRTKPKRQPRYHVVLWDDNYHTYQYVVVMLQSLFGHPPERGYRLAKEVDTQGRVIVLTTTREHAELKRDQIHAFGYDRLLAHSKGSMKASIEAEE

SEQ ID NO: 265 - protein, 119 aa, synthetic construct
MPTAASATESAIEDTPAPARPEVDGRTKPKRQPRYHVVLWDDDDHTYQYVVVMLQSLFGHPPERGYRLAKEVDTQGRVIVLTTTREHAELKRDQIHAFGYDRLLAHSKGSMKASIEAEE

SEQ ID NO: 266 - protein, 107 aa, synthetic construct
MPSAAPAKPKTKRQSRTQGMPPYNVVLLDDDDHTYGYVIEMLNKVFGHPPKKGFELATEVDKNGRVIVMTTNLEVAELKRDEVHAFGPDPLMPRSKGSMSAVVERAG

SEQ ID NO: 267 - protein, 107 aa, synthetic construct
MPSAAPAKPKTKRQSRTQGMPPYNVVLLDDDDHTYGYVIEMLNKVFGHPPEKGFELATEVDKNGRVIVMTTNLEVAELKRDEVHAFGPDPLMPHSKGSMSAVVERAG

SEQ ID NO: 268 - protein, 107 aa, synthetic construct
MPSAAPAKPKTKRQSRTQGMPPYNVVLLDDDDHTYGYVIEMLNKVFGHPPKKGFELATEVDKNGRVIVMTTNLEVAELKRDEVHAFGPDPLMPHSKGSMSAVVERAG

SEQ ID NO: 269 - protein, 107 aa, synthetic construct
MPSAAPAKPKTKRQSRTQGMPPYNVVLLDDDDHTYGYVIEMLNKVFGHPPEKGFELATEMDKLGRVIVMTTNLEVAELKRDEVHAFGPDPLMPRSKGSMSAVVERAG

SEQ ID NO: 270 - protein, 107 aa, synthetic construct
MPSAAPAKPKTKRQSRTQGMPPYNVVLLDDNYHTYGYVIEMLNKVFGHPPEKGFELATEVDKNGRVIVMTTNLEVAELKRDEVHAFGPDPLMPRSKGSMSAVVERAG

SEQ ID NO: 271 - protein, 119 aa, synthetic construct
MPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDPLMPRSKGSMSAVVEAEE

SEQ ID NO: 272 - protein, 119 aa, synthetic construct
MPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDPLMPRSKGSMSASIEAEE

SEQ ID NO: 273 - protein, 119 aa, synthetic construct
MPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGPDPLMPRSKGSMSAVVERAG

SEQ ID NO: 274 - protein, 119 aa, synthetic construct
MPTAASATESAFEDTPAPARPVVDGRTKPKHQPRYHVVLWDDDDHTYQYVVVMLRSLFGHPRERGYRMAKEMDTLGRVIVLTTTREHAELKRDQIHAFGYDRLLARSKGSMKASIEAEE

SEQ ID NO: 275 - protein, 119 aa, synthetic construct
MPTAASATESAFEDTPAPARPVVDGRTKPKRQPRYHVVLWDDDDHTYQYVVVMLRSLFGHPRERGYRMAKEMDTLGRVIVLTTTREHAELKRDQIHAFGYDRLLARSKGSMKASIEAEE

SEQ ID NO: 276 - protein, 119 aa, synthetic construct
MPTAASATESAFEDTPAPARPEVDGRTKPKRQPRYHVVLWDDDDHTYQYVVVMLRSLFGHPRERGYRMAKEMDTLGRVIVLTTTREHAELKRDQIHAFGYDRLLARSKGSMKASIEAEE

SEQ ID NO: 277 - protein, 119 aa, synthetic construct
MPTAASATESAFEDTPAPARPEVDGRTKPKRQPRYHVVLWDDDDHTYQYVVVMLRSLFGHPRERGYRMAKEMDTQGRVIVLTTTREHAELKRDQIHAFGYDRLLARSKGSMKASIEAEE

SEQ ID NO: 278 - protein, 119 aa, synthetic construct
MPTAASATESAIEDTPAPARPVVDGRTKPKHQPRYHVVLWDDDDHTYQYVVVMLRSLFGHPRERGYRMAKEMDTLGRVIVLTTTREHAELKRDQIHAFGYDRLLARSKGSMKASIEAEE

SEQ ID NO: 279 - protein, 119 aa, synthetic construct
MPTAASATESAIEDTPAPARPVVDGRTKPKHQPRYHVVLWDDDDHTYQYVVVMLQSLFGHPRERGYRMAKEMDTLGRVIVLTTTREHAELKRDQIHAFGYDRLLARSKGSMKASIEAEE

SEQ ID NO: 280 - protein, 119 aa, synthetic construct
MPTAASATESAIEDTPAPARPVVDGRTKPKHQPRYHVVLWDDDDHTYQYVVVMLQSLFGHPPERGYRMAKEMDTLGRVIVLTTTREHAELKRDQIHAFGYDRLLARSKGSMKASIEAEE

SEQ ID NO: 281 - protein, 119 aa, synthetic construct
MPTAASATESAFEDTPAPARPEVDGRTKPKRQPRYHVVLWDDDDHTYQYVVVMLRSLFGHPPERGYRMAKEMDTQGRVIVLTTTREHAELKRDQIHAFGYDRLLARSKGSMKASIEAEE

SEQ ID NO: 282 - protein, 119 aa, synthetic construct
MPTAASATESAIEDTPAPARPEVDGRTKPKRQPRYHVVLWDDDDHTYQYVVVMLQSLFGHPRERGYRMAKEMDTLGRVIVLTTTREHAELKRDQIHAFGYDRLLARSKGSMKASIEAEE

SEQ ID NO: 283 - protein, 119 aa, synthetic construct
MPTAASATESAIEDTPAPARPEVDGRTKPKHQPRYHVVLWDDDDHTYQYVVVMLRSLFGHPPERGYRMAKEMDTQGRVIVLTTTREHAELKRDQIHAFGYDRLLARSKGSMKASIEAEE

SEQ ID NO: 284 - protein, 119 aa, synthetic construct
MPTAASATESAIEDTPAPARPVVDGRTKPKHQPRYHVVLWDDDDHTYQYVVVMLQSLFGHPPERGYRMAKEMDTQGRVIVLTTTREHAELKRDQIHAFGYDRLLARSKGSMKASIEAEE

SEQ ID NO: 285 - protein, 119 aa, synthetic construct
MPTAASATESAIEDTPAPARPVVDGRTKPKRQPRYHVVLWDDDDHTYQYVVVMLQSLFGHPPERGYRMAKEMDTQGRVIVLTTTREHAELKRDQIHAFGYDRLLARSKGSMKASIEAEE

SEQ ID NO: 286 - protein, 119 aa, synthetic construct
MPTAASATESAIEDTPAPARPEVDGRTKPKRQPRYHVVLWDDDDHTYQYVVVMLRSLFGHPPERGYRMAKEMDTQGRVIVLTTTREHAELKRDQIHAFGYDRLLARSKGSMKASIEAEE

SEQ ID NO: 287 - protein, 119 aa, synthetic construct
MPTAASATESAIEDTPAPARPEVDGRTKPKRQPRYHVVLWDDDDHTYQYVVVMLQSLFGHPRERGYRMAKEMDTQGRVIVLTTTREHAELKRDQIHAFGYDRLLARSKGSMKASIEAEE

SEQ ID NO: 288 - protein, 119 aa, synthetic construct
MPTAASATESAIEDTPAPARPEVDGRTKPKRQPRYHVVLWDDDDHTYQYVVVMLQSLFGHPPERGYRMAKEMDTLGRVIVLTTTREHAELKRDQIHAFGYDRLLARSKGSMKASIEAEE

SEQ ID NO: 289 - protein, 119 aa, synthetic construct; identical to SEQ ID NO: 286

SEQ ID NO: 290 - protein, 119 aa, synthetic construct
MPTAASATESAFEDTPAPARPEVDGRTKPKRQPRYHVVLWDDDDHTYQYVVVMLQSLFGHPPERGYRMAKEMDTQGRVIVLTTTREHAELKRDQIHAFGYDRLLARSKGSMKASIEAEE

SEQ ID NO: 291 - protein, 119 aa, synthetic construct
MPTAASATESAIEDTPAPARPEVDGRTKPKHQPRYHVVLWDDDDHTYQYVVVMLQSLFGHPPERGYRMAKEMDTQGRVIVLTTTREHAELKRDQIHAFGYDRLLARSKGSMKASIEAEE

SEQ ID NO: 292 - protein, 119 aa, synthetic construct
MPTAASATESAFEDTPAPARPVVDGRTKPKHQPRYHVVLWDDDDHTYQYVVVMLRSLFGHPRERGYRLAKEMDTLGRVIVLTTTREHAELKRDQIHAFGYDRLLARSKGSMKASIEAEE

SEQ ID NO: 293 - protein, 119 aa, synthetic construct
MPTAASATESAFEDTPAPARPVVDGRTKPKRQPRYHVVLWDDDDHTYQYVVVMLRSLFGHPRERGYRLAKEMDTLGRVIVLTTTREHAELKRDQIHAFGYDRLLARSKGSMKASIEAEE

SEQ ID NO: 294 - protein, 119 aa, synthetic construct
MPTAASATESAFEDTPAPARPEVDGRTKPKRQPRYHVVLWDDDDHTYQYVVVMLRSLFGHPRERGYRLAKEMDTLGRVIVLTTTREHAELKRDQIHAFGYDRLLARSKGSMKASIEAEE

SEQ ID NO: 295 - protein, 119 aa, synthetic construct
MPTAASATESAFEDTPAPARPEVDGRTKPKRQPRYHVVLWDDDDHTYQYVVVMLRSLFGHPRERGYRLAKEMDTQGRVIVLTTTREHAELKRDQIHAFGYDRLLARSKGSMKASIEAEE

SEQ ID NO: 296 - protein, 119 aa, synthetic construct
MPTAASATESAIEDTPAPARPVVDGRTKPKHQPRYHVVLWDDDDHTYQYVVVMLRSLFGHPRERGYRLAKEMDTLGRVIVLTTTREHAELKRDQIHAFGYDRLLARSKGSMKASIEAEE

SEQ ID NO: 297 - protein, 119 aa, synthetic construct
MPTAASATESAIEDTPAPARPVVDGRTKPKHQPRYHVVLWDDDDHTYQYVVVMLQSLFGHPRERGYRLAKEMDTLGRVIVLTTTREHAELKRDQIHAFGYDRLLARSKGSMKASIEAEE

SEQ ID NO: 298 - protein, 119 aa, synthetic construct
MPTAASATESAIEDTPAPARPVVDGRTKPKHQPRYHVVLWDDDDHTYQYVVVMLQSLFGHPPERGYRLAKEMDTLGRVIVLTTTREHAELKRDQIHAFGYDRLLARSKGSMKASIEAEE

SEQ ID NO: 299 - protein, 119 aa, synthetic construct
MPTAASATESAIEDTPAPARPVVDGRTKPKHQPRYHVVLWDDDDHTYQYVVVMLQSLFGHPPERGYRLAKEMDTQGRVIVLTTTREHAELKRDQIHAFGYDRLLARSKGSMKASIEAEE

SEQ ID NO: 300 - protein, 119 aa, synthetic construct
MPTAASATESAFEDTPAPARPEVDGRTKPKRQPRYHVVLWDDDDHTYQYVVVMLQSLFGHPPERGYRLAKEMDTQGRVIVLTTTREHAELKRDQIHAFGYDRLLARSKGSMKASIEAEE

SEQ ID NO: 301 - protein, 119 aa, synthetic construct
MPTAASATESAIEDTPAPARPEVDGRTKPKHQPRYHVVLWDDDDHTYQYVVVMLQSLFGHPPERGYRLAKEMDTQGRVIVLTTTREHAELKRDQIHAFGYDRLLARSKGSMKASIEAEE

SEQ ID NO: 302 - protein, 119 aa, synthetic construct
MPTAASATESAFEDTPAPARPVVDGRTKPKHQPRYHVVLWDDNYHTYQYVVVMLRSLFGHPRERGYRMAKEMDTLGRVIVLTTTREHAELKRDQIHAFGYDRLLAHSKGSMKASIEAEE

SEQ ID NO: 303 - protein, 119 aa, synthetic construct
MPTAASATESAIEDTPAPARPEVDGRTKPKRQPRYHVVLWDDNYHTYQYVVVMLQSLFGHPPERGYRMAKEMDTQGRVIVLTTTREHAELKRDQIHAFGYDRLLAHSKGSMKASIEAEE

SEQ ID NO: 304 - protein, 119 aa, synthetic construct
MPTAASATESAIEDTPAPARPEVDGRTKPKRQPRYHVVLWDDDYHTYQYVVVMLQSLFGHPPERGYRMAKEMDTQGRVIVLTTTREHAELKRDQIHAFGYDRLLAHSKGSMKASIEAEE

SEQ ID NO: 305 - protein, 119 aa, synthetic construct
MPTAASATESAIEDTPAPARPEVDGRTKPKRQPRYHVVLWDDDDHTYQYVVVMLQSLFGHPPERGYRMAKEMDTQGRVIVLTTTREHAELKRDQIHAFGYDRLLAHSKGSMKASIEAEE

SEQ ID NO: 306 - protein, 119 aa, synthetic construct
MPTAASATESAIEDTPAPARPEVDGRTKPKRQPRYHVVLWDDDYHTYQYVVVMLQSLFGHPPERGYRMAKEMDTQGRVIVLTTTREHAELKRDQIHAFGYDRLLARSKGSMKASIEAEE

SEQ ID NO: 307 - protein, 119 aa, synthetic construct
MPTAASATESAIEDTPAPARPEVDGRTKPKRQPRYHVVLWDDNDHTYQYVVVMLQSLFGHPPERGYRMAKEMDTQGRVIVLTTTREHAELKRDQIHAFGYDRLLARSKGSMKASIEAEE

SEQ ID NO: 308 - protein, 119 aa, synthetic construct; identical to SEQ ID NO: 292

SEQ ID NO: 309 - protein, 119 aa, synthetic construct
MPTAASATESAIEDTPAPARPEVDGRTKPKHQPRYHVVLWDDDDHTYQYVVVMLRSLFGHPPERGYRLAKEMDTLGRVIVLTTTREHAELKRDQIHAFGYDRLLARSKGSMKASIEAEE

SEQ ID NO: 310 - protein, 119 aa, synthetic construct
MPTAASATESAIEDTPAPARPEVDGRTKPKHQPRYHVVLWDDDDHTYQYVVVMLRSLFGHPRERGYRLAKEMDTQGRVIVLTTTREHAELKRDQIHAFGYDRLLARSKGSMKASIEAEE

SEQ ID NO: 311 - protein, 119 aa, synthetic construct
MPTAASATESAIEDTPAPARPVVDGRTKPKHQPRYHVVLWDDDDHTYQYVVVMLRSLFGHPPERGYRLAKEMDTQGRVIVLTTTREHAELKRDQIHAFGYDRLLARSKGSMKASIEAEE

SEQ ID NO: 312 - protein, 144 aa, synthetic construct
MHHHHHHHHHHDYDIPTTENLYFQGMPTAASATESAIEDTPAPARPEVDGRTKPKHQPRYHVVLWDDDDHTYQYVVVMLRSLFGHPPERGYRLAKEMDTQGRVIVLTTTREHAELKRDQIHAFGYDRLLARSKGSMKASIEAEE

SEQ ID NO: 313 - protein, 119 aa, synthetic construct
MPTAASATESAIEDTPAPARPEVDGRTKPKRQPRYHVVLWDDDDHTYQYVVVMLRSLFGIPPERGYRLAKEMDTQGRVIVLTTTREHAELKRDQIHAFGYDRLLARSKGSMKASIEAEE

SEQ ID NO: 314 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 315 - protein, 119 aa, synthetic construct
MPTAASATESAIEDTPAPARPEVDGRTKPKRQPRYHVVLWDDDDHTYQYVVVMLRSLFGHPYARGYRLAKEMDTQGRVIVLTTTREHAELKRDQIHAFGRDRLLARSKGSMKASIEAEE

SEQ ID NO: 316 - protein, 119 aa, synthetic construct
MPTAASATESAIEDTPAPARPEVDGRTKPKRQPRYHVVLWDDDDHTYQYVVVMLRSLFGHPPARGYRLAKEMDTQGRVIVLTTTREHAELKRDQIHAFGRDRLLARSKGSMKASIEAEE

SEQ ID NO: 317 - protein, 119 aa, synthetic construct
MPTAASATESAIEDTPAPARPEVDGRTKPKRQPRYHVVLWDDDDHTYQYVVVMLRSLFGHPPWRGYRLAKEMDTQGRVIVLTTTREHAELKRDQIHAFGYDRLLAHSKGSMKASIEAEE

SEQ ID NO: 318 - protein, 119 aa, synthetic construct
MPTAASATESAIEDTPAPARPEVDGRTKPKRQPRYHVVLWDDDDHTYQYVVVMLQSLFGHPPSRGYRMAKEMDTQGRVIVLTTTREHAELKRDQIHAFGYDRLLAHSKGSMKASIEAEE

SEQ ID NO: 319 - protein, 119 aa, synthetic construct
MPTAASATESAIEDTPAPARPEVDGRTKPKRQPRYHVVLWDDDDHTYQYVVVMLQSLFGHPPARGYRLAKEMDTQGRVIVLTTTREHAELKRDQIHAFGYDRLLAHSKGSMKASIEAEE

SEQ ID NO: 320 - protein, 119 aa, synthetic construct; identical to SEQ ID NO: 316

SEQ ID NO: 321 - protein, 119 aa, synthetic construct
MPTAASATESAIEDTPAPARPEVDGRTKPKRQPRYHVVLWNDDDHTYQYVVVMLRSLFGHPPERGYRLAKEMDTQGRVIVLTTTREHAELKRDQIHAFGRDRLLARSKGSMKASIEAEE

SEQ ID NO: 322 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLAHSKGSMKASIEAEE

SEQ ID NO: 323 - protein, 119 aa, synthetic construct
MPTAASATESAIEDTPAPARPEVDGRTKPKRQPRYHVVLWNDDDHTYQYVVVMLRSLFGHPPSRGYRLAKEMDTQGRVIVLTTTREHAELKRDQIHAFGYDRLLARSKGSMKASIEAEE

SEQ ID NO: 324 - protein, 119 aa, synthetic construct
MPTAASATESAIEDTPAPARPEVDGRTKPKHQPRYHVVLWDDDDHTYQYVVVMLRSLFGHPPERGYRLAKEMDTQGRVIVLTTTREHAELKRDQIHAFGYDRLLAHSKGSMKASIEAEE

SEQ ID NO: 325 - protein, 119 aa, synthetic construct
MPTAASATESAIEDTPAPARPEVDGRTKPKHQPRYHVVLWDDDDHTYQYVVVMLHSLFGHPPERGYRLAKEMDTQGRVIVLTTTREHAELKRDQIHAFGYDRLLAHSKGSMKASIEAEE

SEQ ID NO: 326 - protein, 119 aa, synthetic construct
MPTAASATESAIEDTPAPARPEVDGRTKPKHQPRYHVVLWDDDDHTYQYLVVMLRSLFGHPPERGYRLAKEMDTQGRVIVLTTTREHAELKRDQIHAFGYDRLLARSKGSMKASIEAEE

SEQ ID NO: 327 - protein, 119 aa, synthetic construct
MPTAASATESAIEDTPAPARPEVDGRTKPKHQPRYHVVLWDDDDHTYQYIVVMLRSLFGHPPERGYRLAKEMDTQGRVIVLTTTREHAELKRDQIHAFGYDRLLARSKGSMKASIEAEE

SEQ ID NO: 328 - protein, 119 aa, synthetic construct
MPTAASATESAIEDTPAPARPEVDGRTKPKHQPRYHVVLWDDDDHTYQYVVVMLRSLFGHPPERGYRLAKEFDTQGRVIVLTTTREHAELKRDQIHAFGYDRLLARSKGSMKASIEAEE

SEQ ID NO: 329 - protein, 119 aa, synthetic construct
MPTAASATESAIEDTPAPARPEVDGRTKPKHQPRYHVVLWDDNYHTYQYVVVMLRSLFGHPPERGYRLAKEMDTQGRVIVLTTTREHAELKRDQIHAFGYDRLLARSKGSMKASIEAEE

SEQ ID NO: 330 - protein, 119 aa, synthetic construct
MPTAASATESAIEDTPAPARPEVDGRTKPKRQPRYHVVLWDDDDHTYQYVVVMLHSLFGHPPERGYRLAKEMDTQGRVIVLTTTREHAELKRDQIHAFGYDRLLARSKGSMKASIEAEE

SEQ ID NO: 331 - protein, 119 aa, synthetic construct
MPTAASGTESAIEDTPAPARPEVDGRTKPKRQPRYHVVLWDDDDHTYQYVVEMLRSLFGHPPERGYRLAKEMDTQGRVIVLTTTREHAELKRDQIHAFGYDRLLARSKGSMKASIEAEE

SEQ ID NO: 332 - protein, 119 aa, synthetic construct
MPTAASATESAFEDTPAPARPEVDGRTKPKRQPRYHVVLWDDDDHTYQYVVVMLRSLFGHPTERGYRLAKEMDTQGRVIVLTTTREHAELKRDQIHAFGYDRLLARSKGSMKASIEAEE

SEQ ID NO: 333 - protein, 119 aa, synthetic construct
MPTAASATESAIEDTPAPARPEVDGRTKPKRQPRYHVVLWDDDDHTYQYVVVMLRSLFGHPPDRGYRLAKEMDTQGRVIVLTTTREHAELKRDQIHAFGYDRLLARSKGSMKASIEAEE

SEQ ID NO: 334 - protein, 119 aa, synthetic construct; identical to SEQ ID NO: 217

SEQ ID NO: 335 - protein, 119 aa, synthetic construct
MPTAASATESAIEDTPAPARPEMDGRTKPKRQPRYHVVLWDDDDHTYQYVVVMLRSLFGHPPERGYRLAKEMDTQGRVIVLTTTREHAELKRGQIHAFGYDRLLARSKGSMKASIEAEE

SEQ ID NO: 336 - protein, 119 aa, synthetic construct
MPTAASATESAIEDTPAPARPEVDGRTKPKRQPRYYVVLWDDDDHTYQYVVVMLRSLFGHPPERGYRLAKEMDTQGRVIVLTTTREHAELKRDQIHAFGYDRLLARSKGSMKASIEAEE

SEQ ID NO: 337 - protein, 119 aa, synthetic construct
MPTAASATESAIEDTPAPARPEVDGRTKPKRQTRYHVVLWDDDDHTYQYVVVMLQSLFGHPPERGYRLAKEMDTQGRVIVLTTTREHAELKRDQIHAFGYDRLLARSKGSMKASIEAEE

SEQ ID NO: 338 - protein, 119 aa, synthetic construct
MPTAASATESAIEDTPAPARPEVDGRTVPKRQPRYHVVLWDDDDHTYQYVVVMLQSLFGHPPERGYHLAKEMDTQGRVIVLTTTREHAELKRDQIHAFGYDRLLARSKGSMKASIEAEE

SEQ ID NO: 339 - protein, 119 aa, synthetic construct
MPTAASATESAIEDTPAPARPEVDGRTKPKRQPRYHVVLWDDDDHTYQYVVVMLQSLFGHPPERGYRLAKEMDTQGRVVVLTTTREHAELKRDQIHAFGYDRLLARSKGSMKASIEAEE

SEQ ID NO: 340 - protein, 119 aa, synthetic construct
MPTAASATESAIEDTPAPARPEVDGRTVPKRQPRYHVVLWDDDDHTYQYVVVMLQSLFGHPPERGYRMAKEMDTQGRVIVLTTTREHAELKRDQIHAFGYDRLLARSKGSMKASIEAEE

SEQ ID NO: 341 - protein, 119 aa, synthetic construct
MPTAASATESAIEDTPAPARPEVDGRTKPRRQPRYHVVLWDDDDHTYQYVVVMLRSLFGHPPERGYRLAKEMDTQGRVIVLTTTREHAELKRDQIHAFGYDRLLARSKGSMKASIEAEE

SEQ ID NO: 342 - protein, 119 aa, synthetic construct
MPTAASATESAIEDTPAPARPEVDGRTVPKRQPRYHVVLWDDDDHTYQYVVVMLQSLFGHPPERGYRLAKEMDTQGRVIVLTTTREHAELKRDQIHAFGYDRLLARSKGSMKASIEAEE

SEQ ID NO: 343 - protein, 119 aa, synthetic construct
MPTAASATESAFEDTPAPARPEVDGRTKPIRQPRYHVVLWDDDDHTYQYVVVMLRSLFGHPPERGYRLAKEMDTQGRVIVLTTTREHAELKRDQIHAFGYDRLLARSKGSMKASIEAEE

SEQ ID NO: 344 - protein, 119 aa, synthetic construct
MPTAASATESAIEDTPAPARPEVDGRTKPKRQPRCHVVLWDDDDHTYQYVVVMLRSLFGHPPERGYRLAKEMDTQGRVIVLTTTREHAELKRDQIHAFGYDRLLARSKGSMKASIEAEE

SEQ ID NO: 345 - protein, 119 aa, synthetic construct
MPTAASATESAIEDTPAPARSEVDGRTKPKRQPRYHVVLWDDDDHTYQYVVVMLQSLFGHPPERGYRLAKEMDTQGRVIVLTTTREHAELKRDQIHAFGYDRLLARSKGSMKASIEAEE

SEQ ID NO: 346 - protein, 119 aa, synthetic construct
MPTAASATESAIEDTPAPARPEVDGRTKPERQPRYHVVLWDDDDHTYQYVVVMLQSLFGHPPERGYRLAKEMDTQGRVIVLTTTREHAELKRDQIHAFGYDRLLARSKGSMKASIEAEE

SEQ ID NO: 347 - protein, 119 aa, synthetic construct
MPTAASATESAIEDTPAPARPEVDGCTKPKRQPRYHVVLWDDDDHTYQYVVVMLQSLFGHPPERGYRLAKEMDTQGRVIVLTTTREHAELKRDQIHAFGYDRLLARSKGSMKASIEAEE

SEQ ID NO: 348 - protein, 119 aa, synthetic construct
MPTAASATESAIEDTPAPARPEVDGSTKPKRQPRYHVVLWDDDDHTYQYVVVMLQSLFGHPPERGYRLAKEMDTQGRVIVLTTTREHAELKRDQIHAFGYDRLLARSKGSMKASIEAEE

SEQ ID NO: 349 - protein, 119 aa, synthetic construct
MPTAASATESAIEDTPAPARPEMDGRTKPKRQPRYHVVLWDDDDHTYQYVVVMLRSLFGHPPERGYRLAKEMDTQGRVIVLTTTREHAELKRDQIHAVGYDRLLARSKGSMKASIEAEE

SEQ ID NO: 350 - protein, 119 aa, synthetic construct
MPTAASATESAIEDTPAPARPEVDGRTKPKRHPRYHVVLWDDDDHTYQYVVVMLQSLFGHSPKRGYCLAKEMDTQGRVIVLTTTREHAELKRDQIHAFGYDRLLARSKGSMKASIEAEE

SEQ ID NO: 351 - protein, 119 aa, synthetic construct
MPTAASATESAFEDTPAPARPEVDGRTKPKRQPRYHVVLWDDDDHTYQYVVVMLRSLFGYPPERGYRLAKEMDTQGRVIVLTTTREHAELKRDQIHAFGYDRLLARSKGSMKASIEAEE

SEQ ID NO: 352 - protein, 119 aa, synthetic construct
MPTAASATESAIEDTPAPARPVVDGRTKPKRQPRYHVVLWDDDDHTYQYVVVMLRSLFGHPPERGYRLAKEMDTQGRVIVLTTTREHAELKRDQIHAFGYDRLLARSKGSMKASIEAEE

SEQ ID NO: 353 - protein, 119 aa, synthetic construct
MPTAASATESAFEDTPAPARPEVDGRTKPKRQPRYHVVLWDDDDHTYQYVVVMLRTLFGHPPERGYRLAKEMDTQGRVIVLTTTREHAELKRDQIHAFGYDRLLARSKGSMKASIEAEE

SEQ ID NO: 354 - protein, 119 aa, synthetic construct
MPTAASATESAIEDTPAPARPEVDGRTKPKHQPRYHVVLWDDDDHTYQYVVVMLRDLFGHPPERGYRLAKEMDTQGRVIVLTTTREHAELKRDQIHAFGYDRLLARSKGSMKASIEAEE

SEQ ID NO: 355 - protein, 119 aa, synthetic construct
MPTAASATESAIEDTPAPARPEVDGRTKPKHQPRYHVVLWDDDDHTYQYVVVMLRSLFGHPKQRGYRLAKEMDTQGRVIVLTTTREHAELKRDQIHAFGYDRLLARSKGSMKASIEAEE

SEQ ID NO: 356 - protein, 119 aa, synthetic construct
MPTAASATESAIEDTPAPARPVVDGRTKPKHQPRYHVVLWDDDDHTYQYVVVMLRSLFGHPQRRGYRLAKEMDTQGRVIVLTTTREHAELKRDQIHAFGYDRLLARSKGSMKASIEAEE

SEQ ID NO: 357 - protein, 119 aa, synthetic construct
MPTAASATESAIEDTPAPARPVVDGRTKPKRQPRYHVVLWDDDDHTYQYVVVMLLSLFGHPSERGYRLAKEMDTQGRVIVLTTTREHAELKRDQIHAFGYDRLLARSKGSMKASIEAEE

SEQ ID NO: 358 - protein, 119 aa, synthetic construct
MPTAASATESAFEDTPAPARPEVDGRTKPKHQPRYHVVLWDDDDHTYQYVVVMLRSLFGHPPERGYRLAKEMDTQGRVIVLTTTREHAELKRDQIHAFGYDRLLARSKGSMKASIEAEE

SEQ ID NO: 359 - protein, 119 aa, synthetic construct
MPTAASATESAIEDTPAPARPEVDGRTKPKHQPRYHVVLWDDDDHTYQYVVVMLRYLFGHPPERGYRLAKEMDTQGRVIVLTTTREHAELKRDQIHAFGYDRLLARSKGSMKASIEAEE

SEQ ID NO: 360 - protein, 119 aa, synthetic construct
MPTAASATESAIEDTPALARPEVDGRTKPKHQPRYHVVLWDDDDHTYQYVVVMLLSLFGHPPERGYRLAKEMDTQGRVIVLTTTREHAELKRDQIHAFGYDRLLARSKGSMKASIEAEE

SEQ ID NO: 361 - protein, 119 aa, synthetic construct
MPTAASATESAIEDTPAPARPVVDGRTKPKRQPRYHVVLWDDHDHTYQYVVVMLRSLFGHPPERGYRLAKEMDTQGRVIVLTTTREHAELKRDQIHAFGYDRLLARSKGSMKASIEAEE

SEQ ID NO: 362 - protein, 119 aa, synthetic construct
MPTAASATESAIEDTPAPARPEVDGRTKPKRQPRYHVVLWDDDDHTYQYVVVMLRSLFGHPPERGYRKAKELDTQGRVIVLTTTREHAELKRDQIHAFGYDRLLARSKGSMKASIEAEE

SEQ ID NO: 363 - protein, 119 aa, synthetic construct
MPTAASATESAIEDTPAPARPEVDGRTKPKRQPRYHVVLWDDDDHTYQYVVVMLRSLFGHPPERGYRLAKELVTQGRVIVLTTTREHAELKRDQIHAFGYDRLLARSKGSMKASIEAEE

SEQ ID NO: 364 - protein, 119 aa, synthetic construct
MPTAASATESAIEDTPAPARPEVDGRTKPKRQPRYHVVLWDDDDHTYQYVVVMLRTLFGHPPSRGYRMAKEMDTQGRVIVLTTTREHAELKRDQIHAFGRDRLLARSKGSMKASIEAEE

SEQ ID NO: 365 - protein, 119 aa, synthetic construct
MPTAASATESAIEDTPAPARPEVDGRTKPKRQPRYHVVLWDDDDHTYQYVVVMLRDLFGHPPSRGYRMAKEMDTQGRVIVLTTTREHAELKRDQIHAFGRDRLLARSKGSMKASIEAEE

SEQ ID NO: 366 - protein, 119 aa, synthetic construct
MPTAASATESAIEDTPAPARPEVDGRTKPKRQPRYHVVLWDDDDHTYQYVVVMLRYLFGHPPSRGYRMAKEMDTQGRVIVLTTTREHAELKRDQIHAFGRDRLLARSKGSMKASIEAEE

SEQ ID NO: 367 - protein, 119 aa, synthetic construct
MPTAASATESAIEDTPAPARPEVDGRTKPKRQPRYHVVLWDDDDHTYQYVVVMLRSLFGHPKQRGYRMAKEMDTQGRVIVLTTTREHAELKRDQIHAFGRDRLLARSKGSMKASIEAEE

SEQ ID NO: 368 - protein, 119 aa, synthetic construct
MPTAASATESAFEDTPAPARPEVDGRTKPKHQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 369 - protein, 119 aa, synthetic construct
MPTAASATESAIEDTPAPARPEVDGRTKPKRQPRYHVVLWDDDDHTYQYVVVMLRSLFGHPQRRGYRMAKEMDTQGRVIVLTTTREHAELKRDQIHAFGRDRLLARSKGSMKASIEAEE

SEQ ID NO: 370 - protein, 119 aa, synthetic construct
MPTAASATESAIEDTPAPARPVVDGRTKPKHQPRYHVVLWDDDDHTYQYVVVMLRSLFGHPQRRGYRMAKEMDTQGRVIVLTTTREHAELKRDQIHAFGRDRLLARSKGSMKASIEAEE

SEQ ID NO: 371 - protein, 119 aa, synthetic construct
MPTAASATESAIEDTPAPARPEVDGRTKPKRQPRYHVVLWDDDDHTYQYVVVMLRSLFGHPRSRGYRMAKEMDTQGRVIVLTTTREHAELKRDQIHAFGRDRLLARSKGSMKASIEAEE

SEQ ID NO: 372 - protein, 119 aa, synthetic construct
MPTAASATESAFEDIPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 373 - protein, 119 aa, synthetic construct
MPTATSATESAIEDTPAPARPEVDGRTKPKRQPHYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 374 - protein, 119 aa, synthetic construct
MPTAASATESAIEDTPAPARPEVDGRTKPKRQPRYHVVLWDDDDHTYQYVVVMLRSLFGHPRERGYRLAKEMDTQGRVIVLTTTREHAELKRDQIHAFGYDRLLARSKGSMKASIEAEE

SEQ ID NO: 375 - protein, 119 aa, synthetic construct
MPTAASATESAFEDIPAPARPEVDGRTKPKRQPRYHVVLWDDDDHTYQYVVVMLRSLFGHPPERGYRLAKEMDTQGRVIVLTTTREHAELKRDQIHAFGYDRLLARSKGSMKASIEAEE

SEQ ID NO: 376 - protein, 119 aa, synthetic construct
MPTATSATESAIEDTPAPARPEVDGRTKPKRQPHYHVVLWDDDDHTYQYVVVMLRSLFGHPPERGYRLAKEMDTQGRVIVLTTTREHAELKRDQIHAFGYDRLLARSKGSMKASIEAEE

SEQ ID NO: 377 - protein, 119 aa, synthetic construct
MPTAASATESAIEDTPAPARPEVDGRTKPKKQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 378 - protein, 119 aa, synthetic construct
MPTAASATESAIEDTPAPARPEVDGRTKPKRQPRYHVVLWDDDDHTYQYVVVMLRSLFGFPPSRGYRMAKEMDTQGRVIVLTTTREHAELKRDQIHAFGRDRLLARSKGSMKASIEAEE

SEQ ID NO: 379 - protein, 119 aa, synthetic construct
MPTAASATESAIEDIPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 380 - protein, 119 aa, synthetic construct
MPTAASATESAIEDTPAPARPEVDGRTKPKRQPRYHVVLWDDDDHTYQYVVVMFRSLFGHPPSRGYRMAKEMDTQGRVIVLTTTREHAELKRDQIHAFGRDRLLARSKGSMKASIEAEE

SEQ ID NO: 381 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHALGRDRLLARSKGSMKASIEAEE

SEQ ID NO: 382 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTNEHAELKRDQIHAFGRDRLLARSKGSMKASIEAEE

SEQ ID NO: 383 - protein, 119 aa, synthetic construct
MPTAASATESAIEDTPAPARPEVDGRTKPKRQPRYHVVLWDDDDHTYQYVVVMFRSLFGHPPSRGYRMAKEMDTQGRVIVLTTTREHAELKRDQIHALGRDRLLARSKGSMKASIEAEE

SEQ ID NO: 384 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGVVIVLTTTREHAELKRDQIHAFGRDRLLARSKGSMKASIEAEE

SEQ ID NO: 385 - protein, 119 aa, synthetic construct
MPTAASATESAIEDTPAPARPEVDGRTKPKRQPRYHVVLWDDDDHTYQYVVVMLRSLFGHPPSRGYRMAKEMRTQGRVIVLTTTREHAELKRDQIHAFGRDRLLARSKGSMKASIEAEE

SEQ ID NO: 386 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIPLTTTREHAELKRDQIHAFGRDRLLARSKGSMKASIEAEE

SEQ ID NO: 387 - protein, 119 aa, synthetic construct
MPTAASATESAIEDTPAPARPEVDGRTKPKRQPRYHVVNWDDDDHTYQYVVVMLRSLFGHPPSRGYRMAKEMDTQGRVIVLTTTREHAELKRDQIHAFGRDRLLARSKGSMKASIEAEE

SEQ ID NO: 388 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTTEHAELKRDQIHAFGRDRLLARSKGSMKASIEAEE

SEQ ID NO: 389 - protein, 250 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEEGSAGSAAGSGEFMPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 390 - protein, 250 aa, synthetic construct
MPTAASATESAIEDTPAPARPEVDGRTKPKRQPRYHVVLWDDDDHTYQYVVVMLRSLFGHPKQRGYRMAKEMDTQGRVIVLTTTREHAELKRDQIHAFGRDRLLARSKGSMKASIEAEEGSAGSAAGSGEFMPTAASATESAIEDTPAPARPEVDGRTKPKRQPRYHVVLWDDDDHTYQYVVVMLRSLFGHPKQRGYRMAKEMDTQGRVIVLTTTREHAELKRDQIHAFGRDRLLARSKGSMKASIEAEE

SEQ ID NO: 391 - protein, 60 aa, synthetic construct
MSPILGYWKIKGLVQPTRLLLEYLEEKYEEHLYERDEGDKWRNKKFELGLEFPNLPYYID

SEQ ID NO: 392 - protein, 60 aa, synthetic construct; identical to SEQ ID NO: 391

SEQ ID NO: 393 - protein, 166 aa, synthetic construct
MGGLFFNALKNCKENFTVLQTIRQQQSTLNGSWVALLQTRNTLNRAGIRYMMDQNNIGSGSTVAELMESASISLKQAEKNWADYEALPRDPRQSTAAAAEIKRNYDIYHNALAELIQLLGAGKINEFFDQPTQGYQDGFEKQYVAYMEQNDRLHDIAVSDNNASYS

SEQ ID NO: 394 - protein, 166 aa, synthetic construct
MGGLFFNALKNDKENFTVLQTIRQQQSTLNGSWVALLQTRNTLNRAGIRYMMDQNNIGSGSTVAELMESASISLKQAEKNWADYEALPRDPRQSTAAAAEIKRNYDIYHNALAELIQLLGAGKINEFFDQPTQGYQDGFEKQYVAYMEQNDRLHDIAVSDNNASYS

SEQ ID NO: 395 - protein, 119 aa, synthetic construct
MPTAASATESAIEDTPAPARPEVDGRTKPKRQPRYHVVLWDDDDHTYQYYVVMLRSLFGHPPSRGYRMAKEMDTQGRVIVLTTTREHAELKRDQIHAFGRDRLLARSKGSMKASIEAEE

SEQ ID NO: 396 - protein, 119 aa, synthetic construct
MPTAASATESAIEDTPAPARPEVDGRTKPKRQPRYHVVLWDDDDHTYQYYVVMLRSLFGHPPSRGYRMAKEIDTQGRVIVLTTTREHAELKRDQIHAFGRDRLLARSKGSMKASIEAEE

SEQ ID NO: 397 - protein, 119 aa, synthetic construct
MPTAASATESAIEDTPAPARPEVDGRTKPKRQPRYHVVLWEDDDETYQYIVVMLRSLFGHPPSRGYRMAKELDTQGRVIVLTTTREHAELKRDQIHAFGRDRLLARSKGSLKASIEAEE

SEQ ID NO: 398 - protein, 119 aa, synthetic construct
MPTAASATESAIEDTPAPARPEVDGRTKPKRQPRYHVVLWNDDDLTYQYLVVMLRSLFGHPPSRGYRMIKEMDTQGRVIVLTTTREHAELKRDQIHAFGRDRLLARSKGSPKASIEAEE

SEQ ID NO: 399 - protein, 119 aa, synthetic construct
MPTAASATESAIEDTPAPARPEVDGRTKPKRQPRYHVVLWEDDDETYQYLVVMLRSLFGHPPSRGYRMAKEIDTQGRVIVLTTTREHAELKRDQIHAFGRDRLLARSKGSMKASIEAEE

SEQ ID NO: 400 - protein, 119 aa, synthetic construct
MPTAASATESAIEDTPAPARPEVDGRTKPKRQPRYHVVLWDDDDDTYQYLVVMLRSLFGHPPSRGYRMMKEIDTQGRVIVLTTTREHAELKRDQIHAFGRDRLLARSKGSLKASIEAEE

SEQ ID NO: 401 - protein, 119 aa, synthetic construct
MPTAASATESAIEDTPAPARPEVDGRTKPKRQPRYHVVLWDDDDETYQYLVVMLRSLFGHPPSRGYRMVKEADTQGRVIVLTTTREHAELKRDQIHAFGRDRLLARSKGSMKASIEAEE

SEQ ID NO: 402 - protein, 119 aa, synthetic construct
MPTAASATESAIEDTPAPARPEVDGRTKPKRQPRYHVVLWTDDDQTYQYMVVMLRSLFGHPPSRGYRMAKELDTQGRVIVLTTTREHAELKRDQIHAFGRDRLLARSKGSPKASIEAEE

SEQ ID NO: 403 - protein, 119 aa, synthetic construct
MPTAASATESAIEDTPAPARPEVDGRTKPKRQPRYHVVLWSDDDETYQYIVVMLRSLFGHPPSRGYRMIKELDTQGRVIVLTTTREHAELKRDQIHAFGRDRLLARSKGSVKASIEAEE

SEQ ID NO: 404 - protein, 119 aa, synthetic construct
MPTAASATESAIEDTPAPARPEVDGRTKPKRQPRYHVVLWDDDDDTYQYLVVMLRSLFGHPPSRGYRMAKELDTQGRVIVLTTTREHAELKRDQIHAFGRDRLLARSKGSMKASIEAEE

SEQ ID NO: 405 - protein, 119 aa, synthetic construct
MPTAASATESAIEDTPAPARPEVDGRTKPKRQPRYHVVLWDDDDHTYQYVVVMLRSLFGHPPSRGYRMVKEMDTQGRVIVLTTTREHAELKRDQIHAFGRDRLLARSKGSVKASIEAEE

SEQ ID NO: 406 - protein, 119 aa, synthetic construct
MPTAASATESAIEDTPAPARPEVDGRTKPKRQPRYHVVLWEDDDETYQYLVVMLRSLFGHPPSRGYRMVKEIDTQGRVIVLTTTREHAELKRDQIHAFGRDRLLARSKGSWKASIEAEE

SEQ ID NO: 407 - protein, 144 aa, synthetic construct
MHHHHHHHHHHDYDIPTTENLYFQGMPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 408 - protein, 119 aa, synthetic construct
MPTAASATESAIEDTPAPARPEVDGRTEPKRQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 409 - protein, 119 aa, synthetic construct
MPTAASATESAIEDTPAPARSEVDGRTKPERQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 410 - protein, 119 aa, synthetic construct
MPTAASATESAIEDTPAPARPEVDGRTKPKRQPRYHVVLWDDDDHSYQYVVVMLRSLFGHPPSRGYRMAKEMDTQGRVIVLTTTREHAELKRDQIHAFGRDRLLARSKGSMKASIEAEE

SEQ ID NO: 411 - protein, 119 aa, synthetic construct
MPTAASATESAIEDTPAPARPEVDGRTKPKRQPRYHVVLWDDDDHIYQYVVVMLRSLFGHPPSRGYRMAKEMDTQGRVIVLTTTREHAELKRDQIHAFGRDRLLARSKGSMKASIEAEE

SEQ ID NO: 412 - protein, 119 aa, synthetic construct
MPTAASATESAIEDTPAPARPEVDGRTKPKRQPRYHVVLWDDDDHTYQYVVVMLRSLVGHPPSRGYRMAKEMDTQGRVIVLTTTREHAELKRDQIHAFGRDRLLARSKGSMKASIEAEE

SEQ ID NO: 413 - protein, 119 aa, synthetic construct
MPTAASATESAIEDTPAPARPEVDGRTKPKRQPRYHVVLWDDDDHTYQYVVVMLRSLFGHSPSRGYRMAKEMDTQGRVIVLTTTREHAELKRDQIHAFGRDRLLARSKGSMKASIEAEE

SEQ ID NO: 414 - protein, 119 aa, synthetic construct
MPTAASATESAIEDTPAPARPEVDGRTKPKRQPRYHVVLWDDDDHTYQYVVVMLRSLFGHPPSRGYRMAKEMHTQGRVIVLTTTREHAELKRDQIHAFGRDRLLARSKGSMKASIEAEE

SEQ ID NO: 415 - protein, 119 aa, synthetic construct
MPTAASATESAIEDTPAPARPEVDGRTKPKRQPRYHVVLWDDDDHTYQYVVVMLRSLFGHPPSRGYRMAKEMVTQGRVIVLTTTREHAELKRDQIHAFGRDRLLARSKGSMKASIEAEE

SEQ ID NO: 416 - protein, 119 aa, synthetic construct
MPTAASATESAIEDTPAPARPEVDGRTKPKRQPRYHVVLWDDDDHTYQYVVVMLRSLFGHPPSRGYRMAKEMDTLGRVIVLTTTREHAELKRDQIHAFGRDRLLARSKGSMKASIEAEE

SEQ ID NO: 417 - protein, 119 aa, synthetic construct
MPTAASATESAIEDTPAPARPEVDGRTKPKRQPRYHVVLWDDDDHTYQYVVVMLRSLFGHPPSRGYRHAKTMDTQGRVIVLTTTREHAELKRDQIHALGRDRLLARSKGSMKASIEAEE

SEQ ID NO: 418 - protein, 119 aa, synthetic construct
MPTAASATESAIEDTPAPARPEVDGRTKPKRQPRYHVVLWDDDRHTYQYVVVMLRSLFGHPPSRGYRMAKEMDTQGRVIVLTTTREHAELKRDQIHAFGRDRLLARSKGSMKASIEAEE

SEQ ID NO: 419 - protein, 119 aa, synthetic construct
MPTAASATESAIEDTPAPARPEVDGRTKPKRQPRYHVVLWDDDDHTDQYVVVMLRSLFGHPPSRGYRMALEAHTQGRVIVLTTTREHAELKRDQIHAFGRDRLLARSKGSMKASIEAEE

SEQ ID NO: 420 - protein, 119 aa, synthetic construct
MPTAASATESAIEDTPAPARPEVDGRTKPKRQPRYHVVLEDDDDHTYQYVVVMLRSLFGHPPSRGYRMAKEYDTQGRVIVLTTTREHAELKRDQIHAFGRDRLLARSKGSMKASIEAEE

SEQ ID NO: 421 - protein, 119 aa, synthetic construct
MPTAASATESAIEDTPAPARPEVDGRTKPKRQPRYHVVLWDDDDHTYQYIVVMLRSLFGHPPSRGYRMARIMDTQGRVIVLTTTREHAELKRDQIHAFGRDRLLARSKGSMKASIEAEE

SEQ ID NO: 422 - protein, 119 aa, synthetic construct
MPTAASATESAIEDTPAPARPEVDGRTKPKRQPRYHVVLWDDDDHTYQYVVVMLRSLFGHPRQRGYRMAKEMDTQGRVIVLTTTREHAELKRDQIHAFGRDRLLARSKGSMKASIEAEE

SEQ ID NO: 423 - protein, 110 aa, synthetic construct
MAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 424 - protein, 100 aa, synthetic construct
MPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 425 - protein, 88 aa, synthetic construct
MPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 426 - protein, 119 aa, synthetic construct
MPTAASATESAIEDTPAPARPEVDGRTKPKRQPRYHVVLWNDDDHTYQYVVVMLQSLFGHPPERGYRLAKEVDTQGRVIVLTTTREHAELKRDQIHAFGYDPLIDRCKGSMSASIEAEE

SEQ ID NO: 427 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDPLIDRCKGSMSASIEAEE

SEQ ID NO: 428 - protein, 119 aa, synthetic construct
MPTAASATESAIEDTPAPARPEVDGRTKPKRQPRYHVVLWDDDDHTYQYIVVMLRSLFGHPPSRGYRMAKEMDTQGRVIVLTTTREHAELKRDQIHAFGRDRLLARSKGSMKASIEAEE

SEQ ID NO: 429 - protein, 119 aa, synthetic construct
MPTAASATESAIEDTPAPARPEVDGRTKPKRQPRYHVVLWDDDDHTYQYTVVMLRSLFGHPPSRGYRMAKELDTQGRVIVLTTTREHAELKRDQIHAFGRDRLLARSKGSMKASIEAEE

SEQ ID NO: 430 - protein, 119 aa, synthetic construct
MPTAASATESAIEDTPAPARPEVDGRTKPKRQPRYHVVLWNDDDHTYQYLVVMLRSLFGHPPSRGYRMAKELDTQGRVIVLTTTREHAELKRDQIHAFGRDRLLARSKGSMKASIEAEE

SEQ ID NO: 431 - protein, 119 aa, synthetic construct
MPTAASATESAIEDTPAPARPEVDGRTKPKRQPRYHVVLWEDDDHTYQYLVVMLRSLFGHPPSRGYRMAKEYDTQGRVIVLTTTREHAELKRDQIHAFGRDRLLARSKGSNKASIEAEE

SEQ ID NO: 432 - protein, 119 aa, synthetic construct
MPTAASATESAIEDTPAPARPEVDGRTKPKRQPRYHVVLWDDDDHTYQYVVVMLRSLFGHPTERGYRLAKEMDTQGRVIVLTTTREHAELKRDQIHAFGRDRLLARSKGSMKASIEAEE

SEQ ID NO: 433 - protein, 119 aa, synthetic construct
MPTAASATESAIEDTPAPARPEVDGRTKPKRQPRYHVVLWDDDDHTYQYVVVMLRSLFGHPTERGYRMAKEMDTQGRVIVLTTTREHAELKRDQIHAFGRDRLLARSKGSMKASIEAEE

SEQ ID NO: 434 - protein, 119 aa, synthetic construct
MPTAASATESAIEDTPAPARPEVDGRTKPKRQPRYHVVLWDDDDHTYQYVVVMLRSLFGHPPWRGYRLAREMDTQGRVIVLTTTREHAELKRDQIHAFGYDRLLARSKGSMKASIEAEE

SEQ ID NO: 435 - protein, 119 aa, synthetic construct
MPTAASATESAIEDTPAPARPEVDGRTKPKRQPRYHVVLWDDDDHTYQYVVVMLRSLFGHPPWRGYRLAREMDTQGRVIVLTTTREHAELKRDQIHAFGRDRLLARSKGSMKASIEAEE

SEQ ID NO: 436 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSLEASIEAEE

SEQ ID NO: 437 - protein, 119 aa, synthetic construct; identical to SEQ ID NO: 436

SEQ ID NO: 438 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHASGRDRLLARSKGSYKASIEAEE

SEQ ID NO: 439 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHASGRDRLLAHSKGSKKASIEAEE

SEQ ID NO: 440 - protein, 119 aa, synthetic construct
MPTAASATESAIEDTPAPARPEVDGCTKPKRQPRYHVVLWDDDDHTYQYVVVMLQSLFGHPPERGYRLAKEMDTQGCVIVLTTTREHAELKRDQIYAFGYDRLLARSKGSMKASIEAEE

SEQ ID NO: 441 - protein, 119 aa, synthetic construct
MPTAASATESAIEDTPAPARPEVDGRTKPKRQPRYHVVLWDDDDHTYQYVVEMLRTLFGHPPERGYRLAKEMDTQGRVIVLTTTREHAELKRDQIHAFGRDRLLARSKGSMKACIEAEE

SEQ ID NO: 442 - protein, 119 aa, synthetic construct
MPTAASATESAIEDTPAPARPEVDGRAKPKRQPRYHVVLWNDDDHTYQYVVVVLQSLFSHPRERGYRLAKEMDTQGRVIVLTTTREHAELKRDQIHAFGYDRLLARSKGSMKASIEAEE

SEQ ID NO: 443 - protein, 119 aa, synthetic construct
MPTAASATESAIGDTPAPARPKMDGRTKPKRQPRYHVVLWNDDDHTYQYAVVMLQSLFGHPPERGYRQAKEVDTQGRVIVLTTTREHAELKRDQIHAFGYDRLLARSKGSMKASIEAEE

SEQ ID NO: 444 - protein, 119 aa, synthetic construct
MPTAASATESAIGDTPAPARPEVDGRTKPKRQPRYHVVLWDDDDHTYQYVVVMLRDLFGHPPERGYHMAKEMDTQGRVIVLTTTREHAELKRDQIHAFGRDRLLARSKGSMKASIEAEE

SEQ ID NO: 445 - protein, 140 aa, synthetic construct
MGSSHHHHHHSSGENLYFQGHMPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 446 - protein, 110 aa, synthetic construct
MGSSHHHHHHSSGENLYFQGHMQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 447 - protein, 83 aa, synthetic construct
MHSKFSHAGRICGAKFKVREPIYRCKECSFDDTCVLCVNCFNPKDHTGHHVYTTICTEFNNGICDCGDKEAWNHTLFCKAEEG

SEQ ID NO: 448 - protein, 83 aa, synthetic construct
MHSKFSHAGRICGAKFKVGEPIYRCKECQFDDTCVLCVNCFNPKDHTGHHVYTTICTEFNNGICDCGDKEAWNHTLFCKAEEG

SEQ ID NO: 449 - protein, 83 aa, synthetic construct
MHSKFSHAGRICGAKFKVGEPIYRCKECSFDYTCVLCVNCFNPKDHTGHHVYTTICTEFNNGICDCGDKEAWNHTLFCKAEEG

SEQ ID NO: 450 - protein, 83 aa, synthetic construct
MHSKFSHAGRICGAKFKVGEPIYRCKECSFDDTCVLCVNCFNPKDHRGHHVYTTICTEFNNGICDCGDKEAWNHTLFCKAEEG

SEQ ID NO: 451 - protein, 83 aa, synthetic construct
MHSKFSHAGRICGAKFKVGEPIYRCKECSFDDTCVLCVNCFNPKDHTRHHVYTTICTEFNNGICDCGDKEAWNHTLFCKAEEG

SEQ ID NO: 452 - protein, 83 aa, synthetic construct
MHSKFSHAGRICGAKFKVGEPIYRCKECSFDDTCVLCVNCFNPKDHTGHHVYVTICTEFNNGICDCGDKEAWNHTLFCKAEEG

SEQ ID NO: 453 - protein, 83 aa, synthetic construct
MHSKFSHAGRICGAKFKVGEPIYRCKECSFDDTCVLCVNCFNPKDHTGHHVYTTICREFNNGICDCGDKEAWNHTLFCKAEEG

SEQ ID NO: 454 - protein, 83 aa, synthetic construct
MHSKFSHAGRICGAKFKVGEPIYRCKECSFDDTCVLCVNCFNPKDHTGHHVYTTICTERNNGICDCGDKEAWNHTLFCKAEEG

SEQ ID NO: 455 - protein, 83 aa, synthetic construct
MHSKFSHAGRICGAKFKVGEPIYRCKECSFDDTCVLCVNCFNPKDHTGHHVYTTICTEFNKGICDCGDKEAWNHTLFCKAEEG

SEQ ID NO: 456 - protein, 83 aa, synthetic construct
MHSKFSHAGRICGAKFKVGEPIYRCKECSFDDTCVLCVNCFNPKDHTGHHVYTTICTEFNNGECDCGDKEAWNHTLFCKAEEG

SEQ ID NO: 457 - protein, 83 aa, synthetic construct
MHSKFSHAGRICGAKFKVGEPIYRCKECSFDDTCVLCVNCFNPKDHTGHHVYTTICTEFNNGICDCGDKSAWNHTLFCKAEEG

SEQ ID NO: 458 - protein, 83 aa, synthetic construct
MHSKFSHAGRICGAKFKVGEPIYRCKECSFDDTCVLCVNCFNPKDHTGHHVYTTICTEFNNGICDCGDKTAWNHTLFCKAEEG

SEQ ID NO: 459 - protein, 83 aa, synthetic construct
MHSKFSHAGRICGAKFKVGEPIYRCKECSFDDTCVLCVNCFNPKDHTGHHVYTTICTEFNNGICDCGDKEAWNKTLFCKAEEG

SEQ ID NO: 460 - protein, 83 aa, synthetic construct
MHSKFSHAGRICGAKFKVGEPIYRCKECSFDDTCVLCVNCFNPKDHRGHHVYTTICTERNNGICDCGDKEAWNHTLFCKAEEG

SEQ ID NO: 461 - protein, 83 aa, synthetic construct
MHSKFSHAGRICGAKFKVGEPIYRCKECSFDDTCVLCVNCFNPKDHRGHHVYTTICTERNNGICDCGDKEAWNHELFCKAEEG

SEQ ID NO: 462 - protein, 83 aa, synthetic construct
MHSKFSHAGRICGAKFKVGEPIYRCKECSFDDTCVLCVNCFNPKDHTYHHVYTTICTEFNNGICDCGDKEAWNHTLFCKAEEG

SEQ ID NO: 463 - protein, 83 aa, synthetic construct
MHSKFSHAGRICGAKFKVGEPIYRCKECSFDDTCVLCVNCFNPKDHLGHHVYTTICTEFNNGICDCGDKEAWNHTLFCKAEEG

SEQ ID NO: 464 - protein, 83 aa, synthetic construct
MHSKFSHAGRICGAKFKVGEPIYRCKECSFDDTCVLCVNCFNPKDHTGHHVYTTICTKFNNGICDCGDKEAWNHTLFCKAEEG

SEQ ID NO: 465 - protein, 83 aa, synthetic construct
MHSKFSHAGRICGAKFKVGEPIYRCKECSFDDTCVLCVNCFNPKDHTGHHVYTTICKEFNNGICDCGDKEAWNHTLFCKAEEG

SEQ ID NO: 466 - protein, 83 aa, synthetic construct
MHSKFSHAGRICGAKFKVGEPIYRCKECSFDDTCVLCVNCFNPKDHTGHHVYTKICTEFNNGICDCGDKEAWNHTLFCKAEEG

SEQ ID NO: 467 - protein, 83 aa, synthetic construct
MHSKFSHAGRICGAKFKVGEPIYRCKECSFDDTCVLCVNCFNPKDHKGHHVYTTICTEFNNGICDCGDKEAWNHTLFCKAEEG

SEQ ID NO: 468 - protein, 83 aa, synthetic construct
MHSKFSHAGRICGAKFKVGEPIYRCKECSFDDTCVLCVNCFNPKDHLGHHVYTTICTEFNNGECDCGDKTAWNHTLFCKAEEG

SEQ ID NO: 469 - protein, 87 aa, synthetic construct
PLYQVVLLDDDDHTYDYIIEMLQQIFIFTMVEGYRRAEELERKGRSVLIVCELSEAEFARDQIPSYGSDWRLPHSQGSMSAVIEPAE

SEQ ID NO: 470 - protein, 119 aa, synthetic construct
MPTAASATESAIEDTPAPARPEVDGRTKPKRQPRYHVVLWDDSDHTRQYAVVMLRSLFGHPPSRGYRMAKEMATQGRVIVLTTTREHAELKRDQIHAFGRDRLLARSKGSMKASIEAEE

SEQ ID NO: 471 - protein, 119 aa, synthetic construct
MPTAASATESAIEDTPAPARPEVDGRTKPKRQPRYHVVLWDDRDHTRQYAVVMLRSLFGHPPSRGYRMAKEIRTQGRVIVLTTTREHAELKRDQIHAFGRDRLLARSKGSMKASIEAEE

SEQ ID NO: 472 - protein, 119 aa, synthetic construct
MPTAASATESAIEDTPAPARPEVDGRTKPKRQPRYHVVLWNDRDHTSQYIVVMLRSLFGHPPSRGYRMAKEMDTQGRVIVLTTTREHAELKRDQIHAFGRDRLLARSKGSMKASIEAEE

SEQ ID NO: 473 - protein, 119 aa, synthetic construct
MPTAASATESAIEDTPAPARPEVDGRTKPKRQPRYHVVLWNDRDHTSQYIIVMLRSLFGHPPSRGYRMAKELQTQGRVIVLTTTREHAELKRDQIHAFGRDRLLARSKGSMKASIEAEE

SEQ ID NO: 474 - protein, 119 aa, synthetic construct
MPTAASATESAIEDTPAPARPEVDGRTKPKRQPRYHVVFWDDDDHTYQYVVVMLRSLFGHPPSRGYRMAHEMCTQGRVIVLTTTREHAELKRDQIHAFGRDRLLARSKGSMKASIEAEE

SEQ ID NO: 475 - protein, 119 aa, synthetic construct
MPTAASATESAIEDTPAPARPEVDGRTKPKRQPRYHVVLWDDDDHTYQYVVVMLRSLFYHPPSRGYRMAKEMDTQGRVIVLTTTREHAELKRDQIHAFGRDRLLARSKGSMKASIEAEE

SEQ ID NO: 476 - protein, 119 aa, synthetic construct
MPTAASATESAIEDTPAPARPEVDGRTKPKRQPRYHVVLWDDDDHTYQYVVVMLRSLFYHPPSRGYRMAHEMDTQGRVIVLTTTREHAELKRDQIHAFGRDRLLARSKGSMKASIEAEE

SEQ ID NO: 477 - protein, 119 aa, synthetic construct
MPTAASATESAIEDTPAPARPEVDGRTKPKRQPRYHVVQWDDDDHTYQYFVVMLRSLFGHPPSRGYRMAKEMDTQGRVIVLTTTREHAELKRDQIHAFGRDRLLARSKGSMKASIEAEE

SEQ ID NO: 478 - protein, 119 aa, synthetic construct
MPTAASATESAIEDTPAPARPEVDGRTKPKRQPRYHVVQIDDDDHTYQYVVVMLRSLFYHPPSRGYRMAKEMDTQGRVIVLTTTREHAELKRDQIHAFGRDRLLARSKGSMKASIEAEE

SEQ ID NO: 479 - protein, 119 aa, synthetic construct
MPTAASATESAIEDTPAPARPEVDGRTVPQRQPRYHVVLWDDDDHTYQYVVGMLRSLFGHPPSRGYRMAKEMDTQGRVIVLTTTREHAELKRDQIHAFGRDRLLARSKGSMKASIEAEE

SEQ ID NO: 480 - protein, 119 aa, synthetic construct
MPTAASATESAIEDIPAPARPEVDGRTKPKHQPRYHVVLWDDDDHTYQYVVVMLRSLFGHPPSRGYRMASEMDTQGRVIVLTTTREHAELKRDQIHAFGRDRLLARSKGSMKASIEAEE

SEQ ID NO: 481 - protein, 119 aa, synthetic construct
MPTAASATESAIEDTPAPARTEVDGRTVPKRQPRYHVVLWDDDDHTYQYVVEMLRSLFGHPPSRGYRMAKEMDTQGRVIVLTTTREHAELKRDQIHAFGRDRLLARSKGSMKASIEAEE

SEQ ID NO: 482 - protein, 119 aa, synthetic construct
MPTAASATESAIEDTPAPARPEVDGRTRPKRQPRYHVVLWDDDDHTYQYVVVMLRKLFGHPPSRGYRMAKEMDTQGRVIVLTTTREHAELKRDQIHAFGRDRLLARSKGSMKASIEAEE

SEQ ID NO: 483 - protein, 119 aa, synthetic construct
MPTAASATESAIEDTPAPARSEVDGYTVPKRQPRYHVVLWDDDDHTYQYVVVMLRSLFGHPPSRGYRMAKEMDTQGRVIVLTTTREHAELKRDQIRAFGRDRLLARSKGSMKASIEAEE

SEQ ID NO: 484 - protein, 167 aa, synthetic construct
MSGSKFRGHQKSKGNSYDVEVVLQHVDTGNSYLCGYLKIKGLTEEYPTLTVFFEGEIISKKHPFLTRKWDADEDVDRKHWGKFLAFYQYAKSFNSDDFDYEELKNGDYVFMRWKEQFLVPDHTIKDISGLSFAGFYYICFQKSAASIEGYYYHRSSEWYQSLNLTHV

SEQ ID NO: 485 - protein, 167 aa, synthetic construct
MSGSKFRGHQKSKGNSYDVEVVLQHVDTGNSYLCGYLKIKGLTEEYPTLTVFFEGEIISKKHPFLTRKWDADEDVDRKHWGKFLAFYQYAKSFNSDDFDYEELKNGDYVFMRWKEQFLVPDHTIKDISGASFAGFYYICFQKSAASIEGYYYHRSSEWYQSLNLTHV

SEQ ID NO: 486 - protein, 167 aa, synthetic construct
MSGSKFRGHQKSKGNSYDVEVVLQHVDTGNSYLCGYLKIKGLTEEYPTLTAFFEGEIISKKHPFLTRKWDADEDVDRKHWGKFLAFYQYAKSFNSDDFDYEELKNGDYVFMRWKEQFLVPDHTIKDISGLSFAGFYYICFQKSAASIEGYYYHRSSEWYQSLNLTHV

SEQ ID NO: 487 - protein, 119 aa, synthetic construct
MPTAASATESAIEDTPAPARPEVDGRTKPKRQPRYHVVLWDDDDHTYQYVVVMLRSLFGHPPSRGYRMAHEMDTQGRVIVLTTTREHAELKRDQIHAFGRDRLLARSKGSMKASIEAEE

SEQ ID NO: 488 - protein, 119 aa, synthetic construct
MPTAASATESAIEDTPAPARPEVDGRTKPKRQPRYHVVLISDDDHTYQYTVVMLRSLFGHPPSRGYRMAHEMDTQGRVIVLTTTREHAELKRDQIHAFGRDRLLARSKGSMKASIEAEE

SEQ ID NO: 489 - protein, 119 aa, synthetic construct
MPTAASATESAIEDTPAPARPEVDGRTKPKRQPRYHVVFWDDDDHTYQYTVVMLRSLFYHPPSRGYRMAHEMCTQGRVIVLTTTREHAELKRDQIHAFGRDRLLARSKGSMKASIEAEE

SEQ ID NO: 490 - protein, 119 aa, synthetic construct
MPTAASATESAIEDTPAPARPEVDGRTKPKRQPRYHVVFWDDDDHTYQYTVVMLRSLFYHPPSRGYRMAHEMDTQGRVIVLTTTREHAELKRDQIHAFGRDRLLARSKGSMKASIEAEE

SEQ ID NO: 491 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRFIVLTTTREHAELKRDQIHAFGRDRLLARSKGSMKASIEAEE

SEQ ID NO: 492 - protein, 119 aa, synthetic construct
MPTAASATESAIEDTPAPARPEVDGRTKPKRQPRYHVVEWDDDSHTYQYVVVMLRSLFGHPPSRGYRMDKEMDTQGRVIVLTTTREHAELKRDQIHAFGRDRLLARSKGSEKASIEAEE

SEQ ID NO: 493 - protein, 119 aa, synthetic construct
MPTAASATESAIEDTPAPARPEVDGRTKPKRQPRYHVVLWDDVDHTYQYTVVMLRSLFGYPPSRGYRMAKEVDTQGRVIVLTTTREHAELKRDQIHAFGRDRLLARSKGSMKASIEAEE

SEQ ID NO: 494 - protein, 119 aa, synthetic construct
MPTAASATESAIEDTPAPARPEVDGRTKPKRQPRYHVVLWDDDDRTYQYVVVMLRSLFGHPPSRGYRMDKEMDTQGRVIVLTTTREHAELKRDQIHAFGRDRLLARSKGSMKASIEAEE

SEQ ID NO: 495 - protein, 119 aa, synthetic construct
MPTAASATESAIEDTPAPARPEVDGRTKPKRQPRYHVVLWDDKDHTPQYVVVMLRSLFGHPPSRGYRMAKEMDTQGRVIVLTTTREHAELKRDQIHAFGRDRLLARSKGSMKASIEAEE

SEQ ID NO: 496 - protein, 119 aa, synthetic construct
MPTAASATESAIEDTPAPARPEVDGRTKPKRQPRYHVVLWPDDDHTYQYVVVMLRSLFGHPPSRGYRLAKEMDTQGRVIVLTTTREHAELKRDQIHAFGRDRLLARSKGSMKASIEAEE

SEQ ID NO: 497 - protein, 119 aa, synthetic construct
MPTAASATESAIEDTPAPARPEVDGRTKPKRQPRYHVVLWDDDDCTYQYLVVMLRSLFGHPPSRGYREAKEMDTQGRRIVLTTTREHAELKRDQIHAFGRDRLLARSKGSMKASIEAEE

SEQ ID NO: 498 - protein, 119 aa, synthetic construct
MPTAASATESAIEDTPAPARPEVDGRTKPKRQPRYHVVLWDDDDHTYQYVVVMLRSLFGHPPSRGYRMIKEMDTQGRVIVLTTTREHAELKRDQIHAFGRDRLLARSKGSMKASIEAEE

SEQ ID NO: 499 - protein, 119 aa, synthetic construct
MPTAASATESAIEDTPAPARPEVDGRTKPKRQPRYHVVLWDDDDHTYQYVVVMLRSLFGIPPSRGYRMAKEMDTQGRVIVLTTTREHAELKRDQIHAFGRDRLLARSKGSMKASIEAEE

SEQ ID NO: 500 - protein, 119 aa, synthetic construct
MPTAASATESAIEDTPAPARPEVDGRTKPKRQPRYHVVLWDDDDHTYQYVVEMLRSLFGIPPSRGYRMAKEMDTQGRVIVLTTTREHAELKRDQIHAFGRDRLLARSKGSMKASIEAEE

SEQ ID NO: 501 - protein, 119 aa, synthetic construct
MPTAASATESAIKDTPAPARSEVDGRTKPERQPRYHVVLWDDDDHTYQYVVVMLRSLFGHPTSRGYRMAKEMDTQGRVIVLTTTREHAELKRDQIHAFGRDRLLARSKGSMKASIEAEE

SEQ ID NO: 502 - protein, 119 aa, synthetic construct
MPTAASATGSAIEDTPAPARPEVDGRTKPKRQPRYHVVLWDDDDHTYQYVVVMLRYLFGHPPERGYRMAKEMDTQGRVIVLTTTREHAELKRDQIHAFGRDRLLARSKGSMKASIEAEE

SEQ ID NO: 503 - protein, 119 aa, synthetic construct
MPTAASATESAIEDTPAPARPEVDGRTKPKRQPRYHVVLWSDEDNTKQYIVVMLRSLFGHPPSRGYRMVEELDTQGRVIVLTTTREHAELKRDQIHAFGRDRLLARSKGSIKASIEAEE

SEQ ID NO: 504 - protein, 119 aa, synthetic construct
MPTAASATESAIEDTPAPARPEVDGRTKPKRQPRYHVVVWDDDDNDEDYVVVMLRSLFGHPPSRGYRMAKEMHTQGRVIVLTTTREHAELKRDQIHAFGRDRLLARSKGSLKASIEAEE

SEQ ID NO: 505 - protein, 119 aa, synthetic construct
MPTAASATESAIEDTPAPARPEVDGRTKPKRQPRYHVVLWDDEDHTYDYIVVMLRSLFGHPPSRGYRMAKELDTQGRVIVLTTTREHAELKRDQIHAFGRDRLLARSKGSVKASIEAEE

SEQ ID NO: 506 - protein, 119 aa, synthetic construct
MPTAASATESAIEDTPAPARPEVDGRTKPKRQPRYHVVLWDDDDHTRQYLVVMLRSLFGHPPSRGYRMTEEADTQGRVIVLTTTREHAELKRDQIHAFGRDRLLARSKGSMKASIEAEE

SEQ ID NO: 507 - protein, 119 aa, synthetic construct
MPTAASATESAIEDTPAPARPEVDGRTKPKRQPRYHVVIWDDEDHTHDYWVVMLRSLFGHPPSRGYRMSEELDTQGRVIVLTTTREHAELKRDQIHAFGRDRLLARSKGSMKASIEAEE

SEQ ID NO: 508 - protein, 126 aa, synthetic construct
MGDVQPETCRPSAASGNYFPQYPEYAIETARLRTFEAWPRNLKQKPHQLAEAGFFYTGVGDRVRCFSCGGGLMDWNDNDEPWEQHARHLSQCRFVKLMKGQLYIDTVAAKPVLAEEKEESTSIGGD

SEQ ID NO: 509 - protein, 116 aa, synthetic construct
MGSDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVR

SEQ ID NO: 510 - protein, 116 aa, synthetic construct
MGSDAVSSDRNFPNSTNLPRNPSMADYEARIFTFGTWIYSVNKEQLARAGFYALGEGDKVKCFHCGGGLTDWKPSEDPWEQHARHYPGCKYLLEQKGQEYINNIHLTHSLEECLVR

SEQ ID NO: 511 - protein, 115 aa, synthetic construct
MGLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSG

SEQ ID NO: 512 - protein, 115 aa, synthetic construct
MGLENSLETLRFSISNLSMQTHAARMRTFMYWPSSVPVQPEQLASAGFYYVGRNDDVKCFCCDGGLRCWESGDDPWVEHARHFPRCEFLIRMKGQEFVDEIQGRYPHLLEQLLSG

SEQ ID NO: 513 - protein, 98 aa, synthetic construct
MGSMRYTVSNLSMQTHAARFKTFFNWPSSVLVNPEQLASAGFYYVGNSDDVKCFCCDGGLRCWESGDDPWVQHAKWFPRCEYLIRIKGQEFIRQVQAS

SEQ ID NO: 514 - protein, 98 aa, synthetic construct
MGSMRYTVSNLSMQTHAARFKTFFNWPSSVLVNPEQLASAGFYYVGNSDDVKCFCCDGGLRCWESGDDPWVQHARHFPRCEYLIRIKGQEFIRQVQAS

SEQ ID NO: 515 - protein, 124 aa, synthetic construct
MGSHMLETEEEEEEGAGATLSRGPAFPGMGSEELRLASFYDWPLTAEVPPELLAAAGFFHTGHQDKVRCFFCYGGLQSWKRGDDPWTEHAKWFPSCQFLLRSKGRDFVHSVQETHSQLLGSWDP

SEQ ID NO: 516 - protein, 124 aa, synthetic construct
MGSHMLETEEEEEEGAGATLSRGPAFPGMGSEELRLASFYDWPLTAEVPPELLAAAGFFHTGHQDKVRCFFCYGGLQSWKRGDDPWTEHARHFPSCQFLLRSKGRDFVHSVQETHSQLLGSWDP

SEQ ID NO: 517 - protein, 109 aa, synthetic construct
MGSHMSTNLPRNPSMTGYEARLITFGTWMYSVNKEQLARAGFYAIGQEDKVQCFHCGGGLANWKPKEDPWEQHAKWYPGCKYLLEEKGHEYINNIHLTRSLEGALVQTT

SEQ ID NO: 518 - protein, 109 aa, synthetic construct
MGSHMSTNLPRNPSMTGYEARLITFGTWMYSVNKEQLARAGFYAIGQEDKVQCFHCGGGLANWKPKEDPWEQHARHYPGCKYLLEEKGHEYINNIHLTRSLEGALVQTT

SEQ ID NO: 519 - protein, 95 aa, synthetic construct
MGSHMRYQEEEARLASFRNWPFYVQGISPCVLSEAGFVFTGKQDTVQCFSCGGCLGNWEEGDDPWKEHAKWFPKCEFLRSKKSSEEITQYIQSYK

SEQ ID NO: 520 - protein, 95 aa, synthetic construct
MGSHMRYQEEEARLASFRNWPFYVQGISPCVLSEAGFVFTGKQDTVQCFSCGGCLGNWEEGDDPWKEHARHFPKCEFLRSKKSSEEITQYIQSYK

SEQ ID NO: 521 - protein, 119 aa, synthetic construct
MPTAASATESAIEDTPAPARPEVDGRTKPKRQPRYHVVLWDDDDHTVQYIVVMLRSLFGHPPSRGYRMAKEMHTQGRVIVLTTTREHAELKRDQIHAFGRDRLLARSKGSLKASIEAEE

SEQ ID NO: 522 - protein, 119 aa, synthetic construct
MPTAASATESAIEDTPAPARPEVDGRTKPKRQPRYHVVLWDDDDHTVQYIVVMLRSLFGHPPSRGYRMAKEATTQGRVIVLTTTREHAELKRDQIHAFGRDRLLARSKGSAKASIEAEE

SEQ ID NO: 523 - protein, 119 aa, synthetic construct
MPTAASATESAIEDTPAPARPEVDGRTKPKRQPRYHVVLWDDEDHTMQYIVVMLRSLFGHPPSRGYRMAKEMHTQGRVIVLTTTREHAELKRDQIHAFGRDRLLARSKGSAKASIEAEE

SEQ ID NO: 524 - protein, 119 aa, synthetic construct
MPTAASATESAIEDTPAPARPEVDGRTKPKRQPRYHVVLWDDDDHTVQYIVVMLRSLFGHPPSRGYRMAKEIGTQGRVIVLTTTREHAELKRDQIHAFGHDRLLARSKGSAKASIEAEE

SEQ ID NO: 525 - protein, 119 aa, synthetic construct
MPTAASATESAIEDTPAPARPEVDGRTKPKRQPRYHVVLWDDDDHTVQYIVVMLRSLFGHPPSRGYRMAKEMHTQGRVIVLTTTREHAELKRDQIHAFGHDRLLARSKGSAKASIEAEE

SEQ ID NO: 526 - protein, 119 aa, synthetic construct
MPTAASATESAIEDTPAPARPEVDGRTKPKRQPRYHVVLWDDDDHTVQYIVVMLRSLFGHPPSRGYRMAKEIYTQGRVIVLTTTREHAELKRDQIHAFGRDRLLARSKGSAKASIEAEE

SEQ ID NO: 527 - protein, 119 aa, synthetic construct
MPTAASATESAIEDTPAPARPEVDGRTKPKRQPRYHVVLWDDDDHTVQYIVVMLRSLFGHPPSRGYRMAKEMHTQGRVIVLTTTREHAELKRDQIHAFGRDRLLARSKGSAKASIEAEE

SEQ ID NO: 528 - protein, 119 aa, synthetic construct
MPTAASATESAIEDTPAPARPEVDGRTKPKRQPRYHVVLWDDRDHTYQYIVVMLRSLFGHPPSRGYRMAKEAYTQGRVIVLTTTREHAELKRDQIHAFGRDRLLARSKGSLKASIEAEE

SEQ ID NO: 529 - protein, 119 aa, synthetic construct
MPTAASATESAIEDTPAPARPEVDGRTKPKRQPRYHVVLWDDRDHTAQYAVVMLRSLFGHPPSRGYRMAKEIYTQGRVIVLTTTREHAELKRDQIHAFGHDRLLARSKGSAKASIEAEE

SEQ ID NO: 530 - protein, 119 aa, synthetic construct
MPTAASATESAIEDTPAPARPEVDGRTKPKRQPRYHVVLWDDNDHTLQYIVVMLRSLFGHPPSRGYRMAKEVDTQGRVIVLTTTREHAELKRDQIHAFGRDRLLARSKGSMKASIEAEE

SEQ ID NO: 531 - protein, 119 aa, synthetic construct
MPTAASATESAIEDTPAPARPEVDGRTKPKRQPRYHVVLWDDDDHTVQYIVVMLRSLFGHPPSRGYRMAKELDTQGRVIVLTTTREHAELKRDQIHAFGRDRLLARSKGSMKASIEAEE

SEQ ID NO: 532 - protein, 119 aa, synthetic construct
MPTAASATESAIEDTPAPARPEVDGRTKPKRQPRYHVVLWDDGDHTWQYIVVMLRSLFGHPPSRGYRMAKELTTQGRVIVLTTTREHAELKRDQIHAFGRDRLLARSKGSMKASIEAEE

SEQ ID NO: 533 - protein, 119 aa, synthetic construct
MPTAASATESAIEDTPAPARPEVDGRTKPKRQPRYHVVLWDDSDHTFQYIVVMLRSLFGHPPSRGYRMAKELHTQGRVIVLTTTREHAELKRDQIHAFGRDRLLARSKGSMKASIEAEE

SEQ ID NO: 534 - protein, 119 aa, synthetic construct
MPTAASATESAIEDTPAPARPEVDGRTKPKRQPRYHVVLWDDADHTYQYIVVMLRSLFGHPPSRGYRMAKEMHTQGRVIVLTTTREHAELKRDQIHAFGRDRLLARSKGSMKASIEAEE

SEQ ID NO: 535 - protein, 119 aa, synthetic construct
MPTAASATESAIEDTPAPARPEVDGRTKPKRQPRYHVVLWDDDDHTMQYIVVMLRSLFGHPPSRGYRMAKEVTTQGRVIVLTTTREHAELKRDQIHAFGRDRLLARSKGSMKASIEAEE

SEQ ID NO: 536 - protein, 119 aa, synthetic construct
MPTAASATESAIEDTPAPARPEVDGRTKPKRQPRYHVVLWDDSDHTIQYIVVMLRSLFGHPPSRGYRMAKEADTQGRVIVLTTTREHAELKRDQIHAFGRDRLLARSKGSMKASIEAEE

SEQ ID NO: 537 - protein, 119 aa, synthetic construct
MPTAASATESAIEDTPAPARPEVDGRTKPKRQPRYHVVLWDDDDHTWQYIVVMLRSLFGHPPSRGYRMAKEMHTQGRVIVLTTTREHAELKRDQIHAFGRDRLLARSKGSMKASIEAEE

SEQ ID NO: 538 - protein, 119 aa, synthetic construct
MPTAASATESAIEDTPAPARPEVDGRTKPKRQPRYHVVLWDDDDHTVQYIVVMLRSLFGHPPSRGYRMAKEMHTQGRVIVLTTTREHAELKRDQIHAFGRDRLLARSKGSMKASIEAEE

SEQ ID NO: 539 - protein, 119 aa, synthetic construct
MPTAASATESAIEDTPAPARPEVDGRTKPKRQPRYHVVLWDDNDHTLQYIVVMLRSLFGHPPSRGYRMAKEMHTQGRVIVLTTTREHAELKRDQIHAFGRDRLLARSKGSMKASIEAEE

SEQ ID NO: 540 - protein, 119 aa, synthetic construct
MPTAASATESAIEDTPAPARPEVDGRTKPKRQPRYHVVLWDDDDHTVQYIVVMLRSLFGHPPSRGYRMAKEISTQGRVIVLTTTREHAELKRDQIHAFGRDRLLARSKGSMKASIEAEE

SEQ ID NO: 541 - protein, 119 aa, synthetic construct
MPTAASATESAIEDTPAPARPEVDGRTKPKRQPRYHVVLWDDGDHTLQYIVVMLRSLFGHPPSRGYRMAKEMHTQGRVIVLTTTREHAELKRDQIHAFGRDRLLARSKGSMKASIEAEE

SEQ ID NO: 542 - protein, 119 aa, synthetic construct
MPTAASATESAIEDTPAPARPEVDGRTKPKRQPRYHVVLWDDDDHTIQYIVVMLRSLFGHPPSRGYRMAKEMHTQGRVIVLTTTREHAELKRDQIHAFGRDRLLARSKGSMKASIEAEE

SEQ ID NO: 543 - protein, 119 aa, synthetic construct
MPTAASATESAIEDTPAPARPEVDGRTKPKRQPRYHVVLWDDEDHTMQYIVVMLRSLFGHPPSRGYRMAKEMHTQGRVIVLTTTREHAELKRDQIHAFGRDRLLARSKGSLKASIEAEE

SEQ ID NO: 544 - protein, 313 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEEGSAGSAAGSGEFMDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLG

SEQ ID NO: 545 - protein, 444 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEEGSAGSAAGSGEFMPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEEGSAGSAAGSGEFMDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLG

SEQ ID NO: 546 - protein, 477 aa, synthetic construct
MSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGSAGSAAGSGEFMSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGHHHHHHHHHHGGGSGGGSGGGSGLNDFFEAQKIEWHEGGGSGGGSGGGSGLNDFFEAQKIEWHEGSAGSAAGSGEFMDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLG

SEQ ID NO: 547 - protein, 277 aa, synthetic construct
MHSKFSHAGRICGAKFKVGEPIYRCKECSFDDTCVLCVNCFNPKDHTGHHVYTTICTEFNNGICDCGDKEAWNHTLFCKAEEGGSAGSAAGSGEFMDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLG

SEQ ID NO: 548 - protein, 372 aa, synthetic construct
MHSKFSHAGRICGAKFKVGEPIYRCKECSFDDTCVLCVNCFNPKDHTGHHVYTTICTEFNNGICDCGDKEAWNHTLFCKAEEGGSAGSAAGSGEFMHSKFSHAGRICGAKFKVGEPIYRCKECSFDDTCVLCVNCFNPKDHTGHHVYTTICTEFNNGICDCGDKEAWNHTLFCKAEEGGSAGSAAGSGEFMDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLG

SEQ ID NO: 549 - protein, 119 aa, synthetic construct
MPTAASATESAIEDTPAPARPEVDGRTKPKRQPRYHVVLLDDDDHTSQYVVVMLRSLFGHPPSRGYRMSKEMDTQGRAIVLTTTREHAELKRDQIHAFGRDRLLARSKGSMKASIEAEE

SEQ ID NO: 550 - protein, 119 aa, synthetic construct
MPTAASATESAIEDTPAPARPEVDGRTKPKRQPRYHVVLLDDPDHTYQYVVVMLRSLFGHPPSRGYRMAKEMDTQGRVIVLTTTREHAELKRDQIHAFGRDRLLARSKGSMKASIEAEE

SEQ ID NO: 551 - protein, 119 aa, synthetic construct
MPTAASATESAIEDTPAPARPEVDGRTKPKRQPRYHVVQWDDDDHTYQYVVVMLRSLFYHPPSRGYRMAHEMDTQGRVIVLTTTREHAELKRDQIHAFGRDRLLARSKGSMKASIEAEE

SEQ ID NO: 552 - protein, 119 aa, synthetic construct
MPTAASATESAIEDTPAPARPEVDGRTKPKRQPRYHVVLWDDDDHTYQYVVVMLRSLFGHPPSRGYRMAKPMDTQGRVIVLTTTREHAELKRDQIHAFGRDRLLARSKGSMKASIEAEE

SEQ ID NO: 553 - protein, 119 aa, synthetic construct
MPTAASATESAIEDTPAPARPEVDGRTKPKRQPRYHVVLWDDDDHTYQYVVVMLRSLFGHPPSRGYRMAKEMKTQGRVIVLTTTREHAELKRDQIHAFGRDRLLARSKGSMKASIEAEE

SEQ ID NO: 554 - protein, 119 aa, synthetic construct
MPTAASATESAIEDTPAPARPEVDGRTKPKRQPRYHVVLYDDDDHTYQYVVVMLRSLFGHPPSRGYRMAKEMDTQGRVIVLTTTREHAELKRDQIHAFGRDRLLARSKGSMKASIEAEE

SEQ ID NO: 555 - protein, 119 aa, synthetic construct
MPTAASATESAIEDTPAPARPEVDGRTKPKRQPRYHVVLQDDDDHTYQYVVVMLRSLFGHPPSRGYRMAKEMDTQGRVIVLTTTREHAELKRDQIHAFGRDRLLARSKGSMKASIEAEE

SEQ ID NO: 556 - protein, 119 aa, synthetic construct
MPTAASATESAIEDTPAPARPEVDGRTKPKRQPRYHVVLWDDGDHTSQYVVVMLRSLFGHPPSRGYRMAKEMDTQGRVIVLTTTREHAELKRDQIHAFGRDRLLARSKGSMKASIEAEE

SEQ ID NO: 557 - protein, 119 aa, synthetic construct
MPTAASATESAIEDTPAPARPEVDGRTKPKRQPRYHVVLWDDDTHTYQYVVVMLRSLFGHPPSRGYRMAKEMDTQGRVIVLTTTREHAELKRDQIHAFGRDRLLARSKGSMKASIEAEE

SEQ ID NO: 558 - protein, 119 aa, synthetic construct
MPTAASATESAIEDTPAPARPEVDGRTKPKRQPRYHVVLWNDSDHTGQYIVVMLRSLFGHPPSRGYRMAKEKDTQGRVIVLTTTREHAELKRDQIHAFGRDRLLARSKGSMKASIEAEE

SEQ ID NO: 559 - protein, 119 aa, synthetic construct
MPTAASATESAIEDTPAPARPEVDGRTKPKRQPRYHVVLWNDLDHTYQYIVVMLRSLFGHPPSRGYRMAKEMHTQGRVIVLTTTREHAELKRDQIHAFGRDRLLARSKGSMKASIEAEE

SEQ ID NO: 560 - protein, 119 aa, synthetic construct
MPTAASATESAIEDTPAPARPEVDGRTKPKRQPRYHVVLWNDGDHTWQYIVVMLRSLFGHPPSRGYRMAKEMHTQGRVIVLTTTREHAELKRDQIHAFGRDRLLARSKGSMKASIEAEE

SEQ ID NO: 561 - protein, 119 aa, synthetic construct
MPTAASATESAIEDTPAPARPEVDGRTKPKRQPRYHVVLWNDGDHTWQYIVVMLRSLFGHPPSRGYRMAKEAKTQGRVIVLTTTREHAELKRDQIHAFGRDRLLARSKGSMKASIEAEE

SEQ ID NO: 562 - protein, 119 aa, synthetic construct
MPTAASATESAIEDTPAPARPEVDGRTKPKRQPRYHVVLWDDGDHTVQYIVVMLRSLFGHPPSRGYRMAKEMHTQGRVIVLTTTREHAELKRDQIHAFGRDRLLARSKGSMKASIEAEE

SEQ ID NO: 563 - protein, 119 aa, synthetic construct
MPTAASATESAIEDTPAPARPEVDGRTKPKRQPRYHVVLWDDQDHTWQYIVVMLRSLFGHPPSRGYRMAKEMHTQGRVIVLTTTREHAELKRDQIHAFGRDRLLARSKGSMKASIEAEE

SEQ ID NO: 564 - protein, 177 aa, synthetic construct
MHSKFSHAGRICGAKFKVGEPIYRCKECSFDDTCVLCVNCFNPKDHRGHHVYTTICTERNNGICDCGDKEAWNHELFCKAEEGSAGSAAGSGEFMHSKFSHAGRICGAKFKVGEPIYRCKECSFDDTCVLCVNCFNPKDHRGHHVYTTICTERNNGICDCGDKEAWNHELFCKAEEG

SEQ ID NO: 565 - protein, 178 aa, synthetic construct
MHSKFSHAGRICGAKFKVGEPIYRCKECSFDDTCVLCVNCFNPKDHLGHHVYTTICTEFNNGECDCGDKTAWNHTLFCKAEEGGGSGGGSGGGSGMHSKFSHAGRICGAKFKVGEPIYRCKECSFDDTCVLCVNCFNPKDHLGHHVYTTICTEFNNGECDCGDKTAWNHTLFCKAEEG

SEQ ID NO: 566 - protein, 177 aa, synthetic construct
MHSKFSHAGRICGAKFKVGEPIYRCKECSFDDTCVLCVNCFNPKDHLGHHVYTTICTEFNNGECDCGDKTAWNHTLFCKAEEGSAGSAAGSGEFMHSKFSHAGRICGAKFKVGEPIYRCKECSFDDTCVLCVNCFNPKDHLGHHVYTTICTEFNNGECDCGDKTAWNHTLFCKAEEG

SEQ ID NO: 567 - protein, 201 aa, synthetic construct
MHSKFSHAGRICGAKFKVGEPIYRCKECSFDDTCVLCVNCFNPKDHLGHHVYTTICTEFNNGECDCGDKTAWNHTLFCKAEEGSAGSAAGSGEFGSAGSAAGSGEFGSAGSAAGSGEFMHSKFSHAGRICGAKFKVGEPIYRCKECSFDDTCVLCVNCFNPKDHLGHHVYTTICTEFNNGECDCGDKTAWNHTLFCKAEEG

SEQ ID NO: 568 - protein, 251 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEEGGGSGGGSGGGSGMPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 569 - protein, 274 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEEGSAGSAAGSGEFGSAGSAAGSGEFGSAGSAAGSGEFMPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 570 - protein, 119 aa, synthetic construct
MPTAASATESAIEDTPAPARPEVDGRTKPKRQPRYHVVLHDDDDHTYQYVVVMLRSLFGHPPSRGYRMAKEMDTQGRVIVLTTTREHAELKRDQIHAFGRDRLLARSKGSMKASIEAEE

SEQ ID NO: 571 - protein, 119 aa, synthetic construct; identical to SEQ ID NO: 499

SEQ ID NO: 572 - protein, 119 aa, synthetic construct
MPTAASATESAIEDTPAPARPEVDGRTKPKRQPRYHVVLWDDDDHTAQYVVVMLRSLFGHPPSRGYRMAKEMDTQGRVIVLTTTREHAELKRDQIHAFGRDRLLARSKGSMKASIEAEE

SEQ ID NO: 573 - protein, 119 aa, synthetic construct
MPTAASATESAIEDTPAPARPEVDGRTKPKRQPRYHVVLWDDNDHTYQYVVVMLRSLFGHPPSRGYRMAKEMDTQGRVIVLTTTREHAELKRDQIHAFGRDRLLARSKGSMKASIEAEE

SEQ ID NO: 574 - protein, 119 aa, synthetic construct
MPTAASATESAIEDTPAPARPEVDGRTKPKRQPRYHVVLWNDDDHTYQYVVVMLRSLFGHPPSRGYRMAKEMDTQGRVIVLTTTREHAELKRDQIHAFGRDRLLARSKGSMKASIEAEE

SEQ ID NO: 575 - protein, 119 aa, synthetic construct
MPTAASATESAIEDTPAPARPEVDGRTKPKRQPRYHVVLWDDDDHTWQYVVVMLRSLFGHPPSRGYRMAKEMDTQGRVIVLTTTREHAELKRDQIHAFGRDRLLARSKGSMKASIEAEE

SEQ ID NO: 576 - protein, 119 aa, synthetic construct
MPTAASATESAIEDTPAPARPEVDGRTKPKRQPRYHVVLWDDDDHTYQYVVVMLRSLFGHPPSRGYRMAKELDTQGRVIVLTTTREHAELKRDQIHAFGRDRLLARSKGSMKASIEAEE

SEQ ID NO: 577 - protein, 119 aa, synthetic construct
MPTAASATESAIEDTPAPARPEVDGRTKPKRQPRYHVVLWDDDDHTYQYVVVMLRSLFGYPPSRGYRMAKEMDTQGRVIVLTTTREHAELKRDQIHAFGRDRLLARSKGSMKASIEAEE

SEQ ID NO: 578 - protein, 119 aa, synthetic construct
MPTAASATESAIEDTPAPARPEVDGRTKPKRQPRYHVVLWDDDDHTLQYVVVMLRSLFGHPPSRGYRMAKEMDTQGRVIVLTTTREHAELKRDQIHAFGRDRLLARSKGSMKASIEAEE

SEQ ID NO: 579 - protein, 119 aa, synthetic construct
MPTAASATESAIEDTPAPARPEVDGRTKPKRQPRYHVVLWDDDDHTIQYVVVMLRSLFGHPPSRGYRMAKEMDTQGRVIVLTTTREHAELKRDQIHAFGRDRLLARSKGSMKASIEAEE

SEQ ID NO: 580 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHASGRDRLLARSKGSMKASIEAEE

SEQ ID NO: 581 - protein, 119 aa, synthetic construct
MPTAASATESAIEDTPAPARPEVDGRTKPKRQPRYHVVLWDDDDHTYQYVVVMLRSLFGHPLSRGYRMAKEMDTQGRVIVLTTTREHAELKRDQIHAFGRDRLLARSKGSMKASIEAEE

SEQ ID NO: 582 - protein, 119 aa, synthetic construct
MPTAASATESAIEDTPAPARPEVDGRTKPKRQPRYHVVLWDDDQHTYQYVVVMLRSLFGHPPSRGYRMAKEMDTQGRVIVLTTTREHAELKRDQIHAFGRDRLLARSKGSMKASIEAEE

SEQ ID NO: 583 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRTIVLTTTREHAELKRDQIHAFGRDRLLARSKGSMKASIEAEE

SEQ ID NO: 584 - protein, 119 aa, synthetic construct
MPTAASATESAIEDTPAPARPEVDGRTKPKRQPRYHVVLFDDDDHTYQYVVVMLRSLFGHPPSRGYRMAKEMDTQGRVIVLTTTREHAELKRDQIHAFGRDRLLARSKGSMKASIEAEE

SEQ ID NO: 585 - protein, 119 aa, synthetic construct
MPTAASATESAIEDTPAPARPEVDGRTKPKRQPRYHVVLWDDDDHTYQYVVVMLRSLFGHPPSRGYRMTKEMDTQGRVIVLTTTREHAELKRDQIHAFGRDRLLARSKGSMKASIEAEE

SEQ ID NO: 586 - protein, 119 aa, synthetic construct
MPTAASATESAIEDTPAPARPEVDGRTKPKRQPRYHVVLWDDDDHTYQYVVVMLRSLFGSPPSRGYRMAKEMDTQGRLIVLTTTREHAELKRDQIHAFGRDRLLARSKGSMKASIEAEE

SEQ ID NO: 587 - protein, 119 aa, synthetic construct
MPTAASATESAIEDTPAPARPEVDGRTKPKRQPRYHVVLWDDDDHTYQYVVVMLRSLFGVPPSRGYRMAKELDTQGRVIVLTTTREHAELKRDQIHAFGRDRLLARSKGSMKASIEAEE

SEQ ID NO: 588 - protein, 119 aa, synthetic construct
MPTAASATESAIEDTPAPARPEVDGRTKPKRQPRYHVVLWDDDDHTYQYVVVMLRSLFGLPPSRGYRMAHEMDTQGRVIVLTTTREHAELKRDQIHAFGRDRLLARSKGSMKASIEAEE

SEQ ID NO: 589 - protein, 119 aa, synthetic construct
MPTAASATESAIEDTPAPARPEVDGRTKPKRQPRYHVVLYDDDDHTDQYVVVMLRSLFGHPPSRGYRLAEEMDTQGRVIVLTTTREHAELKRDQIHAFGRDRLLARSKGSMKASIEAEE

SEQ ID NO: 590 - protein, 119 aa, synthetic construct
MPTAASATESAIEDTPAPARPEVDGRTKPKRQPRYHVVLLDDDSHTYQYVVVMLRSLFGVPPSRGYRMAAEMDTQGRVIVLTTTREHAELKRDQIHAFGRDRLLARSKGSMKASIEAEE

SEQ ID NO: 591 - protein, 119 aa, synthetic construct
MPTAASATESAIEDTPAPARPEVDGRTKPKRQPRYHVVLWDDHDHTYQYVVVMLRSLFGHPPSRGYRMAKELHTQGRVIVLTTTREHAELKRDQIHAFGRDRLLARSKGSMKASIEAEE

SEQ ID NO: 592 - protein, 204 aa, synthetic construct
MEGNGPAAVHYQPASPPRDACVYSSCYCEENVWKLCEYIKNHDQYPLEECYAVFISNERKMIPIWKQQARPGDGPVIWDYHVVLLHVSSGGQSFIYDLDTVLPFPCLFDTYVEDAIKSDDDIHPQFRRKFRVICADSYLKNFASDRSHMKDSSGNWREPPPPYPCIETGDSKMNLNDFISMDPKVGWGAVYTLSEFTHRFGSKN

SEQ ID NO: 593 - protein, 204 aa, synthetic construct
MEGNGPAAVHYQPASPPRDACVYSSCYSEENVWKLCEYIKNHDQYPLEECYAVFISNERKMIPIWKQQARPGDGPVIWDYHVVLLHVSSGGQSFIYDLDTVLPFPCLFDTYVEDAIKSDDDIHPQFRRKFRVICADSYLKNFASDRSHMKDSSGNWREPPPPYPCIETGDSKMNLNDFISMDPKVGWGAVYTLSEFTHRFGSKN

SEQ ID NO: 594 - protein, 204 aa, synthetic construct
MEGNGPAAVHYQPASPPRDACVYSSCYSEENVWKLCEYIKNHDQYPLEECYAVFISNERKMIPIWKQQARPGDGPVIWDYQVVLLHVSSGGQSFIYDLDTVLPFPCLFDTYVEDAIKSDDDIHPQFRRKFRVICADSYLKNFASDRSHMKDSSGNWREPPPPYPCIETGDSKMNLNDFISMDPKVGWGAVYTLSEFTHRFGSKN

SEQ ID NO: 595 - protein, 204 aa, synthetic construct
MEGNGPAAVHYQPASPPRDACVYSSCYSEENVWKLCEYIKNHDQYPLEECYAVFISNERKMIPIWKQQARPGDGPVIWDYQVVLLHVSSGGQSFIYDLDTVLPFPCLFDTYVEDAIKSDDDIHPQFRRKFRVICADSYLKNFASDRSHEKDSSGNWREPPPPYPCIETGDSKMNLNDFISMDPKVGWGAVYTLSEFTHRFGSKN

SEQ ID NO: 596 - protein, 204 aa, synthetic construct
MGPAATAPQYQPVCPTRDACVYNSCYSEENIWKLCEYIKTHNQYLLEECYAVFISNEKKMVPIWKQQARPENGPVIWDYHVVLLHVSREGQSFIYDLDTILPFPCPFDIYIEDALKSDDDIHLQFRRKFRVVRADSYLKHFASDRSHMKDSSGNWREPPPEYPCIETGDSKMNLNDFISMDPAVGWGAVYTLPEFVHRFSSKTY

SEQ ID NO: 597 - protein, 204 aa, synthetic construct
MGPAATAPQYQPVCPTRDACVYNSCYSEENIWKLCEYIKTHNQYLLEECYAVFISNEKKMVPIWKQQARPENGPVIWDYQVVLLHVSREGQSFIYDLDTILPFPCPFDIYIEDALKSDDDIHLQFRRKFRVVRADSYLKHFASDRSHMKDSSGNWREPPPEYPCIETGDSKMNLNDFISMDPAVGWGAVYTLPEFVHRFSSKTY

SEQ ID NO: 598 - protein, 204 aa, synthetic construct
MGPAATAPQYQPVCPTRDACVYNSCYSEENIWKLCEYIKTHNQYLLEECYAVFISNEKKMVPIWKQQARPENGPVIWDYQVVLLHVSREGQSFIYDLDTILPFPCPFDIYIEDALKSDDDIHLQFRRKFRVVRADSYLKHFASDRSHEKDSSGNWREPPPEYPCIETGDSKMNLNDFISMDPAVGWGAVYTLPEFVHRFSSKTY

SEQ ID NO: 599 - protein, 203 aa, synthetic construct
MVPAAAAARYQPASPPRDACVYNSCYSEENIWKLCEYIKNHDQYPLEECYAVFISNERKMIPIWKQQARPGDGPVIWYYFFLLVRYHVKSIGFSFTFQAIPLVNTLEDILAQLFKFCIHMHACVLWKFRVIRADSYLKNFASDRSHMKDSSGNWREPPPSYPCIETGDSKMNLNDFISMDPEVGWGAVYSLSEFVHRFGSQNY

SEQ ID NO: 600 - protein, 203 aa, synthetic construct
MVPAAAAARYQPASPPRDACVYNSCYSEENIWKLCEYIKNHDQYPLEECYAVFISNERKMIPIWKQQARPGDGPVIWYYFFLLVRYHVKSIGFSFTFQAIPLVNTLEDILAQLFKFCIHMHACVLWKFRVIRADSYLKNFASDRSHEKDSSGNWREPPPSYPCIETGDSKMNLNDFISMDPEVGWGAVYSLSEFVHRFGSQNY

SEQ ID NO: 601 - protein, 199 aa, synthetic construct
MAAGEPSPFLVRSDCLYTSCYSEENVWKLCEYIRDHRPCLLEQFSAVFISNENKMIPIWKQKSAKGDGPVIWDYHVILLHESARDGNFVYDLDTILPFPSPCNTYIREALKCDSNIHCDFRRKLRVVGAHEFLQTFASDRSHMRDSSSNWTKPPPPYPCIQTAESTMNLDDFISMNPEVGWGTVYSLAAFIERFGDTTL

SEQ ID NO: 602 - protein, 199 aa, synthetic construct
MAAGEPSPFLVRSDCLYTSCYSEENVWKLCEYIRDHRPCLLEQFSAVFISNENKMIPIWKQKSAKGDGPVIWDYQVILLHESARDGNFVYDLDTILPFPSPCNTYIREALKCDSNIHCDFRRKLRVVGAHEFLQTFASDRSHMRDSSSNWTKPPPPYPCIQTAESTMNLDDFISMNPEVGWGTVYSLAAFIERFGDTTL

SEQ ID NO: 603 - protein, 199 aa, synthetic construct
MAAGEPSPFLVRSDCLYTSCYSEENVWKLCEYIRDHRPCLLEQFSAVFISNENKMIPIWKQKSAKGDGPVIWDYQVILLHESARDGNFVYDLDTILPFPSPCNTYIREALKCDSNIHCDFRRKLRVVGAHEFLQTFASDRSHERDSSSNWTKPPPPYPCIQTAESTMNLDDFISMNPEVGWGTVYSLAAFIERFGDTTL

SEQ ID NO: 604 - protein, 200 aa, synthetic construct
MNGLSAQHERIAPARHECVYTSCYSEENVWKLCEHIKTSKRCPLGDVYAVFISNERKMVPIWKQKSGRGEEPVIWDYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 605 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 606 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHEKDASGGWRMPPPPYPCIETAESRMNLDDFISMNPSVGWGHVYTLEEFVQHFGKT

SEQ ID NO: 607 - protein, 200 aa, synthetic construct
MESASSEYKVITPSGNQCVYTSCYSEENVWKLCEYIKNQRHCPLEEVYAVFISNERKKIPIWKQKSSRGDEPVIWDYHVILLHASKQGPSFIYDLDTILPFPCSLDVYSMEAFQSDKHLKPAYWRKLRVIPGDTYLKEFASDRSHMKDSDGNWRMPPPAYPCLETPESKMNLDDFICMDPRVGYGEVYSLSDFVKHFGVK

SEQ ID NO: 608 - protein, 200 aa, synthetic construct
MESASSEYKVITPSGNQCVYTSCYSEENVWKLCEYIKNQRHCPLEEVYAVFISNERKKIPIWKQKSSRGDEPVIWDYQVILLHASKQGPSFIYDLDTILPFPCSLDVYSMEAFQSDKHLKPAYWRKLRVIPGDTYLKEFASDRSHMKDSDGNWRMPPPAYPCLETPESKMNLDDFICMDPRVGYGEVYSLSDFVKHFGVK

SEQ ID NO: 609 - protein, 200 aa, synthetic construct
MESASSEYKVITPSGNQCVYTSCYSEENVWKLCEYIKNQRHCPLEEVYAVFISNERKKIPIWKQKSSRGDEPVIWDYQVILLHASKQGPSFIYDLDTILPFPCSLDVYSMEAFQSDKHLKPAYWRKLRVIPGDTYLKEFASDRSHEKDSDGNWRMPPPAYPCLETPESKMNLDDFICMDPRVGYGEVYSLSDFVKHFGVK

SEQ ID NO: 610 - protein, 200 aa, synthetic construct
MEHVSSKYVNITPSRDECVYTSCYSEENVWKLCEHIKTQTQIHLDEVYAVFISNERKMIPIWKQKSSRGDEPVVWDYHVVLLHQNQQGQSFIYDQDTVLPFSCPFHVYTTEAFHTDHGLKPAFWRKLRVIPADTYLKNFASDRSHMKNADGTWRMPPPLYPCIETTDSKMNLDDFISMDSKVGCGHVYSLSEFVKHFAEK

SEQ ID NO: 611 - protein, 200 aa, synthetic construct
MEHVSSKYVNITPSRDECVYTSCYSEENVWKLCEHIKTQTQIHLDEVYAVFISNERKMIPIWKQKSSRGDEPVVWDYQVVLLHQNQQGQSFIYDQDTVLPFSCPFHVYTTEAFHTDHGLKPAFWRKLRVIPADTYLKNFASDRSHMKNADGTWRMPPPLYPCIETTDSKMNLDDFISMDSKVGCGHVYSLSEFVKHFAEK

SEQ ID NO: 612 - protein, 200 aa, synthetic construct
MEHVSSKYVNITPSRDECVYTSCYSEENVWKLCEHIKTQTQIHLDEVYAVFISNERKMIPIWKQKSSRGDEPVVWDYQVVLLHQNQQGQSFIYDQDTVLPFSCPFHVYTTEAFHTDHGLKPAFWRKLRVIPADTYLKNFASDRSHEKNADGTWRMPPPLYPCIETTDSKMNLDDFISMDSKVGCGHVYSLSEFVKHFAEK

SEQ ID NO: 613 - protein, 115 aa, synthetic construct
MGLENSLETLRFSISNLSMQTHAARMRTFMYWPSSVPVQPEQLASAGFYYLGRNDIVICFCCDGGLHCWQSGDDPWVEHALFFPRCEFLIRMKGQEFVDEIQGRYPHLLEQLLSG

SEQ ID NO: 614 - protein, 107 aa, synthetic construct
MHHHHHHHHHHDYDIPTTENLYFQGMHSKFSHAGRICGAKFKVGEPIYRCKECSFDDTCVLCVNCFNPKDHLGHHVYTTICTEFNNGECDCGDKTAWNHTLFCKAEE

SEQ ID NO: 615 - protein, 120 aa, synthetic construct
MHHHHHHHHHHDYDIPTTENLYFQGRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRY

SEQ ID NO: 616 - protein, 264 aa, synthetic construct
MGDVQPETCRPSAASGNYFPQYPEYAIETARLRTFEAWPRNLKQKPHQLAEAGFFYTGVGDRVRCFSCGGGLMDWNDNDEPWEQHARHLSQCRFVKLMKGQLYIDTVAAKPVLAEEKEESTSIGGDGSAGSAAGSGEFMGDVQPETCRPSAASGNYFPQYPEYAIETARLRTFEAWPRNLKQKPHQLAEAGFFYTGVGDRVRCFSCGGGLMDWNDNDEPWEQHARHLSQCRFVKLMKGQLYIDTVAAKPVLAEEKEESTSIGGD

SEQ ID NO: 617 - protein, 241 aa, synthetic construct
MGLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSGSAGSAAGSGEFMGLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSG

SEQ ID NO: 618 - protein, 241 aa, synthetic construct
MGLENSLETLRFSISNLSMQTHAARMRTFMYWPSSVPVQPEQLASAGFYYVGRNDDVKCFCCDGGLRCWESGDDPWVEHARHFPRCEFLIRMKGQEFVDEIQGRYPHLLEQLLSGSAGSAAGSGEFMGLENSLETLRFSISNLSMQTHAARMRTFMYWPSSVPVQPEQLASAGFYYVGRNDDVKCFCCDGGLRCWESGDDPWVEHARHFPRCEFLIRMKGQEFVDEIQGRYPHLLEQLLSG

SEQ ID NO: 619 - protein, 266 aa, synthetic construct
MGDVQPETCRPSAASGNYFPQYPEYAIETARLRTFEAWPRNLKQKPHQLAEAGFFYTGVGDRVRCFSCGGGLMDWNDNDEPWEQHALWLSQCRFVKLMKGQLYIDTVAAKPVLAEEKEESTSIGGDTGSAGSAAGSGEFMGDVQPETCRPSAASGNYFPQYPEYAIETARLRTFEAWPRNLKQKPHQLAEAGFFYTGVGDRVRCFSCGGGLMDWNDNDEPWEQHALWLSQCRFVKLMKGQLYIDTVAAKPVLAEEKEESTSIGGDT

SEQ ID NO: 620 - protein, 242 aa, synthetic construct
MGLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSGGGSGGGSGGGSGMGLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSG

SEQ ID NO: 621 - protein, 265 aa, synthetic construct
MGLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSGSAGSAAGSGEFGSAGSAAGSGEFGSAGSAAGSGEFMGLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSG

SEQ ID NO: 622 - protein, 119 aa, synthetic construct
MPTAASATESAIEDTPAPARPEVDGRTKPKRQPRYHVVLWDDEDHTYQYVVVMLRSLFGHPPSRGYRMAKEMDTQGRVIVLTTTREHAELKRDQIHAFGRDRLLARSKGSMKASIEAEE

SEQ ID NO: 623 - protein, 119 aa, synthetic construct
MPTAASATESAIEDTPAPARPEVDGRTKPKRQPRYHVVLWDDDDHTYQYVVVMLRSLFGHPPSRGYRMAREMDTQGRVIVLTTTREHAELKRDQIHAFGRDRLLARSKGSMKASIEAEE

SEQ ID NO: 624 - protein, 119 aa, synthetic construct
MPTAASATESAIEDTPAPARPEVDGRTKPKRQPRYHVVLWDDDDHTYQYVVVMLRSLFGHPPSRGYRMAKEMTTQGRVIVLTTTREHAELKRDQIHAFGRDRLLARSKGSMKASIEAEE

SEQ ID NO: 625 - protein, 119 aa, synthetic construct
MPTAASATESAIEDTPAPARPEVDGRTKPKRQPRYHVVLWDDQDHTYQYVVVMLRSLFGHPPSRGYRMAKEMDTQGRVIVLTTTREHAELKRDQIHAFGRDRLLARSKGSMKASIEAEE

SEQ ID NO: 626 - protein, 119 aa, synthetic construct
MPTAASATESAIEDTPAPARPEVDGRTKPKRQPRYHVVLWDDDEHTYQYVVVMLRSLFGHPPSRGYRMAKEMDTQGRVIVLTTTREHAELKRDQIHAFGRDRLLARSKGSMKASIEAEE

SEQ ID NO: 627 - protein, 119 aa, synthetic construct
MPTAASATESAIEDTPAPARPEVDGRTKPKRQPRYHVVLWDDDDHTMQYVVVMLRSLFGHPPSRGYRMAKEMDTQGRVIVLTTTREHAELKRDQIHAFGRDRLLARSKGSMKASIEAEE

SEQ ID NO: 628 - protein, 119 aa, synthetic construct
MPTAASATESAIEDTPAPARPEVDGRTKPKRQPRYHVVLWDDDDHTFQYVVVMLRSLFGHPPSRGYRMAKEMDTQGRVIVLTTTREHAELKRDQIHAFGRDRLLARSKGSMKASIEAEE

SEQ ID NO: 629 - protein, 119 aa, synthetic construct
MPTAASATESAIEDTPAPARPEVDGRTKPKRQPRYHVVLWDDDDHTVQYVVVMLRSLFGHPPSRGYRMAKEMDTQGRVIVLTTTREHAELKRDQIHAFGRDRLLARSKGSMKASIEAEE

SEQ ID NO: 630 - protein, 119 aa, synthetic construct
MPTAASATESAIEDTPAPARPEVDGRTKPKRQPRYHVVLYDDDTHTYQYVVVMLRSLFGHPPSRGYRMAKEMDTQGRVIVLTTTREHAELKRDQIHAFGRDRLLARSKGSMKASIEAEE

SEQ ID NO: 631 - protein, 119 aa, synthetic construct
MPTAASATESAIEDTPAPARPEVDGRTKPKRQPRYHVVLYDDEDHTYQYVVVMLRSLFGHPPSRGYRMAKEMDTQGRVIVLTTTREHAELKRDQIHAFGRDRLLARSKGSMKASIEAEE

SEQ ID NO: 632 - protein, 119 aa, synthetic construct
MPTAASATESAIEDTPAPARPEVDGRTKPKRQPRYHVVLYDDDDHTFQYVVVMLRSLFGHPPSRGYRMAKEMDTQGRVIVLTTTREHAELKRDQIHAFGRDRLLARSKGSMKASIEAEE

SEQ ID NO: 633 - protein, 119 aa, synthetic construct
MPTAASATESAIEDTPAPARPEVDGRTKPKRQPRYHVVLYDDDDHTYQYVVVMLRSLFGHPPSRGYRMAREMDTQGRVIVLTTTREHAELKRDQIHAFGRDRLLARSKGSMKASIEAEE

SEQ ID NO: 634 - protein, 119 aa, synthetic construct
MPTAASATESAIEDTPAPARPEVDGRTKPKRQPRYHVVLYDDDDHTYQYVVVMLRSLFGHPPSRGYRMAKEMTTQGRVIVLTTTREHAELKRDQIHAFGRDRLLARSKGSMKASIEAEE

SEQ ID NO: 635 - protein, 119 aa, synthetic construct
MPTAASATESAIEDTPAPARPEVDGRTKPKRQPRYHVVLWDDEDHTYQYVVVMLRSLFGHPPSRGYRMAKEMTTQGRVIVLTTTREHAELKRDQIHAFGRDRLLARSKGSMKASIEAEE

SEQ ID NO: 636 - protein, 119 aa, synthetic construct
MPTAASATESAIEDTPAPARPEVDGRTKPKRQPRYHVVLYDDEDHTFQYVVVMLRSLFGHPPSRGYRMAKEMDTQGRVIVLTTTREHAELKRDQIHAFGRDRLLARSKGSMKASIEAEE

SEQ ID NO: 637 - protein, 119 aa, synthetic construct
MPTAASATESAIEDTPAPARPEVDGRTKPKRQPRYHVVLYDDDDHTYQYVVVMLRSLFGHPPSRGYRMAREMTTQGRVIVLTTTREHAELKRDQIHAFGRDRLLARSKGSMKASIEAEE

SEQ ID NO: 638 - protein, 119 aa, synthetic construct
MPTAASATESAIEDTPAPARPEVDGRTKPKRQPRYHVVLWDDEDHTYQYVVVMLRSLFGHPPSRGYRMAREMTTQGRVIVLTTTREHAELKRDQIHAFGRDRLLARSKGSMKASIEAEE

SEQ ID NO: 639 - protein, 119 aa, synthetic construct
MPTAASATESAIEDTPAPARPEVDGRTKPKRQPRYHVVLYDDEDHTFQYVVVMLRSLFGHPPSRGYRMAKEMTTQGRVIVLTTTREHAELKRDQIHAFGRDRLLARSKGSMKASIEAEE

SEQ ID NO: 640 - protein, 119 aa, synthetic construct
MPTAASATESAIEDTPAPARPEVDGRTKPKRQPRYHVVLWDDEDHTFQYVVVMLRSLFGHPPSRGYRMAREMTTQGRVIVLTTTREHAELKRDQIHAFGRDRLLARSKGSMKASIEAEE

SEQ ID NO: 641 - protein, 119 aa, synthetic construct
MPTAASATESAIEDTPAPARPEVDGRTKPKRQPRYHVVLYDDEDHTFQYVVVMLRSLFGHPPSRGYRMAREMTTQGRVIVLTTTREHAELKRDQIHAFGRDRLLARSKGSMKASIEAEE

SEQ ID NO: 642 - protein, 119 aa, synthetic construct
MPTAASATESAIEDTPAPARPEVDGRTKPKRQPRYHVVLWSDEDHTHQYVVVMLRSLFGHPPSRGYRMAKEIDTQGRVIVLTTTREHAELKRDQIHAFGRDRLLARSKGSMKASIEAEE

SEQ ID NO: 643 - protein, 119 aa, synthetic construct
MPTAASATESAIEDTPAPARPEVDGRTKPKRQPRYHVVIWEDDDHTYQYWVVMLRSLFGHPPSRGYRMAKEAHTQGRVIVLTTTREHAELKRDQIHAFGRDRLLARSKGSLKASIEAEE

SEQ ID NO: 644 - protein, 119 aa, synthetic construct
MPTAASATESAIEDTPAPARPEVDGRTKPKRQPRYHVVIWSDDDHTHDYVVVMLRSLFGHPPSRGYRMTKELHTQGRVIVLTTTREHAELKRDQIHAFGRDRLLARSKGSMKASIEAEE

SEQ ID NO: 645 - protein, 119 aa, synthetic construct
MPTAASATESAIEDTPAPARPEVDGRTKPKRQPRYHVVMWDDQDNTDQYWVVMLRSLFGHPPSRGYRMSEELHTQGRVIVLTTTREHAELKRDQIHAFGRDRLLARSKGSVKASIEAEE

SEQ ID NO: 646 - protein, 119 aa, synthetic construct
MPTAASATESAIEDTPAPARPEVDGRTKPKRQPRYHVVVWDDEDHTHQYWVVMLRSLFGHPPSRGYRMAKEAHTQGRVIVLTTTREHAELKRDQIHAFGRDRLLARSKGSLKASIEAEE

SEQ ID NO: 647 - protein, 119 aa, synthetic construct
MPTAASATESAIEDTPAPARPEVDGRTKPKRQPRYHVVLWDDDDHTSQYVVVMLHSLFGHPPSRGYRLAKEMDTQGRVIVLTTTREHAELKRDQIHAFGRDRLLARSKGSMKASIEAEE

SEQ ID NO: 648 - protein, 119 aa, synthetic construct
MPTAASATESAIEDTPAPARPEVDGRTKPKRQPRYHVVLWSDEDHTHQYIVVMLRSLFGHPPSRGYRMAKEIDTQGRVIVLTTTREHAELKRDQIHAFGRDRLLARSKGSMKASIEAEE

SEQ ID NO: 649 - protein, 119 aa, synthetic construct
MPTAASATESAIEDTPAPARPEVDGRTKPKRQPRYHVVLWDDDDHTKQYIVVMLRSLFGHPPSRGYRMAKEMHTQGRVIVLTTTREHAELKRDQIHAFGRDRLLARSKGSVKASIEAEE

SEQ ID NO: 650 - protein, 119 aa, synthetic construct
MPTAASATESAIEDTPAPARPEVDGRTKPKRQPRYHVVMWEDEDHTFQYVVVMLRSLFGHPPSRGYRMVKEMHTQGRVIVLTTTREHAELKRDQIHAFGRDRLLARSKGSMKASIEAEE

SEQ ID NO: 651 - protein, 766 aa, synthetic construct
MASDTPESLMALCTDFCLRNLDGTLGYLLDKETLRLHPDIFLPSEICDRLVNEYVELVNAACNFEPHESFFSLFSDPRSTRLTRIHLREDLVQDQDLEAIRKQDLVELYLTNCEKLSAKSLQTLRSFSHTLVSLSLFGCTNIFYEEENPGGCEDEYLVNPTCQVLVKDFTFEGFSRLRFLNLGRMIDWVPVESLLRPLNSLAALDLSGIQTSDAAFLTQWKDSLVSLVLYNMDLSDDHIRVIVQLHKLRHLDISRDRLSSYYKFKLTREVLSLFVQKLGNLMSLDISGHMILENCSISKMEEEAGQTSIEPSKSSIIPFRALKRPLQFLGLFENSLCRLTHIPAYKVSGDKNEEQVLNAIEAYTEHRPEITSRAINLLFDIARIERCNQLLRALKLVITALKCHKYDRNIQVTGSAALFYLTNSEYRSEQSVKLRRQVIQVVLNGMESYQEVTVQRNCCLTLCNFSIPEELEFQYRRVNELLLSILNPTRQDESIQRIAVHLCNALVCQVDNDHKEAVGKMGFVVTMLKLIQKKLLDKTCDQVMEFSWSALWNITDETPDNCEMFLNFNGMKLFLDCLKEFPEKQELHRNMLGLLGNVAEVKELRPQLMTSQFISVFSNLLESKADGIEVSYNACGVLSHIMFDGPEAWGVCEPQREEVEERMWAAIQSWDINSRRNINYRSFEPILRLLPQGISPVSQHWATWALYNLVSVYPDKYCPLLIKEGGMPLLRDIIKMATARQETKEMARKVIEHCSNFKEENMDTSR

SEQ ID NO: 652 - protein, 347 aa, synthetic construct
MLTNSEYRSEQSVKLRRQVIQVVLNGMESYQEVTVQRNCCLTLCNFSIPEELEFQYRRVNELLLSILNPTRQDESIQRIAVHLCNALVCQVDNDHKEAVGKMGFVVTMLKLIQKKLLDKTCDQVMEFSWSALWNITDETPDNCEMFLNFNGMKLFLDCLKEFPEKQELHRNMLGLLGNVAEVKELRPQLMTSQFISVFSNLLESKADGIEVSYNACGVLSHIMFDGPEAWGVCEPQREEVEERMWAAIQSWDINSRRNINYRSFEPILRLLPQGISPVSQHWATWALYNLVSVYPDKYCPLLIKEGGMPLLRDIIKMATARQETKEMARKVIEHCSNFKEENMDTSR

SEQ ID NO: 653 - protein, 347 aa, synthetic construct
MLTNSEYRMEQSIKLRRQVIQVVLNGMESYQEVTVQRNCCLTLCNFSIPEELEFQYRRVNELLLSILNQSRQDESIQRIAVHLCNALVCQVDNDHKEAVGKMGFVMTMLKLIQKKLADKTCDQVMEFSWSALWNITDETPDNCEMFLNYSGMKLFLECLKEFPEKQELHRNMLGLLGNVAEVRELRPQLMTSQFISVFSNLLESKADGIEVSYNACGVLSHIMFDGPEAWGICEPHREEVVKRMWAAIQSWDINSRRNINYRSFEPILRLLPQGISPVSQHWATWALYNLVSVYPDKYCPLLIKEGGIPLLKDMIKMASARQETKEMAWKVIEHCSNFKEENMDTSR

SEQ ID NO: 654 - protein, 355 aa, synthetic construct
MTGSAALFYLTNTEYRGEQSVRLRRQVIQVVLNGMEHYQEVTVQRNCCLTLCNFSIPEELEFQYRRVNLLLLKILEPLRQDESIQRIAVHLCNALVCQVDNDHKEAVGKMGFVKTMLNLIQKKLQDRMCDQVMEFSWSALWNITDETPDNCQMFLECNGMNLFLECLKEFPDKQELHRNMLGLLGNVAEVKALRPQLLTRQFITVFSDLLDSKADGIEVSYNACGVLSHIMFDGPGVWSMEEPSRTHVMDKMWTAIQSWDVSSRRNINYRSFEPILRLLPQSGAPVSQHWATWALYNLVSVYPSKYCPLLIKEGGVSLLQAVLELQTSHVETKDMARKVMEQCESFKEDPMDTSR

SEQ ID NO: 655 - protein, 744 aa, synthetic construct
MPEDQAGAAMEEASPYSLLDICLNFLTTHLEKFCSARQDGTLCLQEPGVFPQEVADRLLRTMAFHGLLNDGTVGIFRGNQMRLKRACIRKAKISAVAFRKAFCHHKLVELDATGVNADITITDIISGLGSNKWIQQNLQCLVLNSLTLSLEDPYERCFSRLSGLRALSITNVLFYNEDLAEVASLPRLESLDISNTSITDITALLACKDRLKSLTMHHLKCLKMTTTQILDVVRELKHLNHLDISDDKQFTSDIALRLLEQKDILPNLVSLDVSGRKHVTDKAVEAFIQQRPSMQFVGLLATDAGYSEFLTGEGHLKVSGEANETQIAEALKRYSERAFFVREALFHLFSLTHVMEKTKPEILKLVVTGMRNHPMNLPVQLAASACVFNLTKQDLAAGMPVRLLADVTHLLLKAMEHFPNHQQLQKNCLLSLCSDRILQDVPFNRFEAAKLVMQWLCNHEDQNMQRMAVAIISILAAKLSTEQTAQLGTELFIVRQLLQIVKQKTNQNSVDTTLKFTLSALWNLTDESPTTCRHFIENQGLELFMRVLESFPTESSIQQKVLGLLNNIAEVQELHSELMWKDFIDHISSLLHSVEVEVSYFAAGIIAHLISRGEQAWTLSRSQRNSLLDDLHSAILKWPTPECEMVAYRSFNPFFPLLGCFTTPGVQLWAVWAMQHVCSKNPSRYCSMLIEEGGLQHLYNIKDHEHTDPHVQQIAVAILDSLEKHIVRHGRPPPCKKQPQARLN

SEQ ID NO: 656 - protein, 359 aa, synthetic construct
MVFNLTKQDLAAGMPVRLLADVTHLLLKAMEHFPNHQQLQKNCLLSLCSDRILQDVPFNRFEAAKLVMQWLCNHEDQNMQRMAVAIISILAAKLSTEQTAQLGTELFIVRQLLQIVKQKTNQNSVDTTLKFTLSALWNLTDESPTTCRHFIENQGLELFMRVLESFPTESSIQQKVLGLLNNIAEVQELHSELMWKDFIDHISSLLHSVEVEVSYFAAGIIAHLISRGEQAWTLSRSQRNSLLDDLHSAILKWPTPECEMVAYRSFNPFFPLLGCFTTPGVQLWAVWAMQHVCSKNPSRYCSMLIEEGGLQHLYNIKDHEHTDPHVQQIAVAILDSLEKHIVRHGRPPPCKKQPQARLN

SEQ ID NO: 657 - protein, 383 aa, synthetic construct
MPEMLKLVVIGMRNHPTNLPVQLAASACVFNLTKQDLAAGMPVKLLADVTHLLLEAMKHFPNHQQLQKNCLLSLCSDRILQDVPFNRFDAAKLVMQWLCNHEDQNMQRMAVAIISILAAKLSTEQTAQLGAELFIVRQLLQIVRQKTSQNMVDTTLKFTLSALWNLTDESPTTCRHFIENQGLELFMKVLETFPSESSIQQKVLGLLNNIAEVKELHSELMCKDFIDQISKLLHSVEVEVSYFAAGIIAHLVSRGEESWTLSSSLRETLLEQLHSAILSWPTPECEMVAYRSFNPFFPLLACFRTPGVQLWAVWAMQHVCSKNPVRYCSMLIEEGGLVRLHRIRDHMCADPDVLRITIAILDNLDRHLRKHGNPPCPKPPFAK

SEQ ID NO: 658 - protein, 397 aa, synthetic construct
MLTHAIEKPRPDILKLVALGMKNHPTTLNVQLAASACVFNLTKQELAFGIPVRLLGNVTQQLLEAMKTFPNHQQLQKNCLLSLCSDRILQEVPFNRFEAAKLVMQWLCNHEDQNMQRMAVAIISILAAKLSTEQTAQLGAELFIVKQLLHIVRQKTCQSTVDATLKFTLSALWNLTDESPTTCRHFIENQGLELFIKVLESFPSESSIQQKVLGLLNNIAEVSELHGELMVQSFLDHIRTLLHSPEVEVSYFAAGILAHLTSRGEKVWTLELTLRNTLLQQLHSAILKWPTPECEMVAYRSFNPFFPLLECFQTPGVQLWAAWAMQHVCSKNAGRYCSMLLEEGGLQHLEAITSHPKTHSDVRRLTESILDGLQRHRARTGYTAIPKTQAHREKCNP

SEQ ID NO: 659 - protein, 420 aa, synthetic construct
MEGNGPAAVHYQPASPPRDACVYSSCYSEENVWKLCEYIKNHDQYPLEECYAVFISNERKMIPIWKQQARPGDGPVIWDYQVVLLHVSSGGQSFIYDLDTVLPFPCLFDTYVEDAIKSDDDIHPQFRRKFRVICADSYLKNFASDRSHMKDSSGNWREPPPPYPCIETGDSKMNLNDFISMDPKVGWGAVYTLSEFTHRFGSKNGSAGSAAGSGEFMEGNGPAAVHYQPASPPRDACVYSSCYSEENVWKLCEYIKNHDQYPLEECYAVFISNERKMIPIWKQQARPGDGPVIWDYQVVLLHVSSGGQSFIYDLDTVLPFPCLFDTYVEDAIKSDDDIHPQFRRKFRVICADSYLKNFASDRSHMKDSSGNWREPPPPYPCIETGDSKMNLNDFISMDPKVGWGAVYTLSEFTHRFGSKN

SEQ ID NO: 660 - protein, 412 aa, synthetic construct
MNGLSAQHERIAPARHECVYTSCYSEENVWKLCEHIKTSKRCPLGDVYAVFISNERKMVPIWKQKSGRGEEPVIWDYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKTGSAGSAAGSGEFMNGLSAQHERIAPARHECVYTSCYSEENVWKLCEHIKTSKRCPLGDVYAVFISNERKMVPIWKQKSGRGEEPVIWDYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 661 - protein, 413 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKTGGGSGGGSGGGSGMNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 662 - protein, 412 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKTGSAGSAAGSGEFMNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 663 - protein, 436 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKTGSAGSAAGSGEFGSAGSAAGSGEFGSAGSAAGSGEFMNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 664 - protein, 119 aa, synthetic construct
MPTAASATESAIEDTPAPARPEVDGRTKPKRQPRYHVVLWDDDDHTYQYVVAMLRSLFGHPPSRGYRMAKEMDTQGRVIVLTTTREHAELKRDQIHAFGRDRLLARSKGSMKASIEAEE

SEQ ID NO: 665 - protein, 119 aa, synthetic construct
MPTAASATESAIEDTPAPARPEVDGRTKPKRQPRYHVVLWDDDDHTYQYVVMMLRSLFGHPPSRGYRMAKEMDTQGRVIVLTTTREHAELKRDQIHAFGRDRLLARSKGSMKASIEAEE

SEQ ID NO: 666 - protein, 119 aa, synthetic construct
MPTAASATESAIEDTPAPARTEVDGRTKPKRQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 667 - protein, 119 aa, synthetic construct
MPTAASATESAIEDTPAPARPEVDGRTKPKRQPRYHVVLWDDDDHTYQYVVAMLRSLFGIPPSRGYRMAKEMDTQGRVIVLTTTREHAELKRDQIHAFGRDRLLARSKGSMKASIEAEE

SEQ ID NO: 668 - protein, 119 aa, synthetic construct
MPTAASATESAIEDTPAPARPEVDGRTKPKRQPRYHVVLWDDDDHTYQYVVMMLRSLFGIPPSRGYRMAKEMDTQGRVIVLTTTREHAELKRDQIHAFGRDRLLARSKGSMKASIEAEE

SEQ ID NO: 669 - protein, 119 aa, synthetic construct
MPTAASATESAIEDTPAPARPEVDGRTKPKRQPRYHVVLWDDDDHTYQYVVMMLRSLFGYPPSRGYRMAKEMDTQGRVIVLTTTREHAELKRDQIHAFGRDRLLARSKGSMKASIEAEE

SEQ ID NO: 670 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAWGRDRLLARSKGSMKASIEAEE

SEQ ID NO: 671 - protein, 119 aa, synthetic construct
MPTAASATESAIEDTPAPARPEVDGRTKPKRQPRYHVVLWDDDDHTYQYVVVMLRSLFGHPKSRGYRMAKEMDTQGRVIVLTTTREHAELKRDQIHAFGRDRLLARSKGSMKASIEAEE

SEQ ID NO: 672 - protein, 119 aa, synthetic construct
MPTAASATESAIEDTPAPARPEVDGRTKPKRQPRYHVVLWDDDDHTYQYVVVMLQSLFGHPPSRGYRMAKEMDTQGRVIVLTTTREHAELKRDQIHAFGRDRLLARSKGSMKASIEAEE

SEQ ID NO: 673 - protein, 119 aa, synthetic construct
MPTAASATESAIEDTPAPARPEVDGRTKPKRQPRYHVVLWDDDDHTYQYVVVMLKSLFGHPPSRGYRMAKEMDTQGRVIVLTTTREHAELKRDQIHAFGRDRLLARSKGSMKASIEAEE

SEQ ID NO: 674 - protein, 119 aa, synthetic construct
MPTAASATESAIEDTPAPARPEVDGRTKPKRQPRYHVVLWDDDDHTYQYVVVMLASLFGHPPSRGYRMAKEMDTQGRVIVLTTTREHAELKRDQIHAFGRDRLLARSKGSMKASIEAEE

SEQ ID NO: 675 - protein, 119 aa, synthetic construct
MPTAASATESAIEDTPAPARPEVDGRTKPKRQPRYHVVLWDDDDHTYQYVVVMLSSLFGHPPSRGYRMAKEMDTQGRVIVLTTTREHAELKRDQIHAFGRDRLLARSKGSMKASIEAEE

SEQ ID NO: 676 - protein, 119 aa, synthetic construct
MPTAASATESAIEDTPAPARPEVDGRTKPKRQPRYHVVLWDDDDHTYQYVVVMLRSLFGHPPQRGYRMAKEMDTQGRVIVLTTTREHAELKRDQIHAFGRDRLLARSKGSMKASIEAEE

SEQ ID NO: 677 - protein, 119 aa, synthetic construct
MPTAASATESAIEDTPAPARPEVDGRTKPKRQPRYHVVLWDDDDHTYQYVVHMLRSLFGHPPSRGYRMAKEMDTQGRVIVLTTTREHAELKRDQIHAFGRDRLLARSKGSMKASIEAEE

SEQ ID NO: 678 - protein, 119 aa, synthetic construct
MPTAASATESAIEDTPAPARPEVDGRTKPKRQPRYHVVLWDDDDHTYQYVVVMLRSVFGHPPSRGYRMAKEMDTQGRVIVLTTTREHAELKRDQIHAFGRDRLLARSKGSMKASIEAEE

SEQ ID NO: 679 - protein, 119 aa, synthetic construct
MPTAASATESAIEDTPAPARPEVDGKTKPKRQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 680 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAYGRDRLLARSKGSMKASIEAEE

SEQ ID NO: 681 - protein, 119 aa, synthetic construct
MPTAASATESAIEDTPAPARSEVDGYTVPKRQQRYHVVLWDDDDHTYQYVVYMLRSVFGHPPSRGYRMAKEMDTQGRVIVLTTTREHAELKRDQIHAFGRDRLLARSKGSMKASIEAEE

SEQ ID NO: 682 - protein, 119 aa, synthetic construct
MPTAASATESAIEDTPAPARPEVDGRTKPKRQPRYHVVLWDDDDHTYQYVVYMLRSVFGHPPSRGYRMAKEMDTQGRVIVLTTTREHAELKRDQIHAFGRDRLLARSKGSMKASIEAEE

SEQ ID NO: 683 - protein, 119 aa, synthetic construct
MPTAASATESAIEDTPAPARPEVDGRTKPKHQPRYHVVLWDDDDHTLQYVVVMLRSLFGHPPSRGYRMAQEMETQGRVIVLTTTREHAELKRDQIHAFGRDRLLARSKGSMKASIEAEE

SEQ ID NO: 684 - protein, 119 aa, synthetic construct
MPTAASATESAIEDTPAPARPEVDGRTKPKRQPRYHVVLWDDDDHTYQYVVEMLRHLFGHPPSRGYRMAKEMDTQGRVIVLTTTREHAELKRDQIHAFGRDRLLARSKGSMKASIEAEE

SEQ ID NO: 685 - protein, 119 aa, synthetic construct
MPTAASATESAIEDTPAPARPEVDGRTKPKRQPRYHVVLWDDDDHTYQYVVSMLRSVFGHPPSRGYRMAKEMDTQGRVIVLTTTREHAELKRDQIRARGRDPLLARSKGSMKASIEAEE

SEQ ID NO: 686 - protein, 119 aa, synthetic construct
MPTAASATESAIEDTPAPARPEVDGRTKPKRQPRYHVVLWDDDDHTYQYVVAMLRSIFGHPPSRGYRMAKEMDTQGRVIVLTTTREHAELKRDQIHAFGRDRLLARSKGSMSASIEAEE

SEQ ID NO: 687 - protein, 119 aa, synthetic construct
MPTAASATESAIEDTPAPARPEVDGRTKPKRQPRYHVVLWDDDDHTYQYVVVMLRSLFGHPPSRGYRMAKELETQGRLIVLTTTREHAELKRDQIHAFGRDRLLARSKGSMKASIEAEE

SEQ ID NO: 688 - protein, 119 aa, synthetic construct
MPTAASATESAIEDTPAPARPEVDGRTKPKHQPRYHVVLWDDDDHTDQYVVVMLRSLFGHPPSRGYRMAKEMDTQGRVIVLTTTREHAELKRDQIHAFGRDRLLARSKGSMSASIEAEE

SEQ ID NO: 689 - protein, 119 aa, synthetic construct
MPTAASATESAIEDTPAPARPEVDGRTKPKRQPRYHVVLWDDDDHTDQYVVVMLRSLFGHPPSRGYRMAKEMDTQGRVIVLTTTREHAELKRDQIHAFGRDRLLARSKGSMSASIEAEE

SEQ ID NO: 690 - protein, 119 aa, synthetic construct
MPTAASATESAIEDTPAPARPEVDGYTVPKRQPRYHVVLWDDDDHTYQYVVVMLRSLFGHPPSRGYRMAKEMDTQGRVIVLTTTREHAELKRDQIHAYGRDRLLARSKGSMKASIEAEE

SEQ ID NO: 691 - protein, 119 aa, synthetic construct
MPTAASATESAIEDTPAPARPEVDGRTKPKRQPRYHVVLWDDDDHTYQYVVTMLRSVFGHPPSRGYRMAKEMDTQGRVIVLTTTREHAELKRDQIHAFGRDRLLARSKGSMKASIEAEE

SEQ ID NO: 692 - protein, 119 aa, synthetic construct
MPTAASATESAIEDTPAPARSEVDGYTVPKRQPRYHVVLWDDDDHTYQYVVIMLRSLFGHPPSRGYRMAKEMDTQGRVTVLTTTREHAELKRDQIHAFGRDRLLARSKGSMKASIEAEE

SEQ ID NO: 693 - protein, 119 aa, synthetic construct
MPTAASATESAIEDTPAPARPEVDGRTKPKRQPRYHVVLWDDDDHTYQYVVIMLRSLFGHPPSRGYRMAKEMDTQGRVTVLTTTREHAELKRDQIHAFGRDRLLARSKGSMKASIEAEE

SEQ ID NO: 694 - protein, 83 aa, synthetic construct
MHSKFSHAGRICGAKFKVGERIYRCKECSFDDTCVLCVNCFNPKDHLGHHVYTTICTEFNNGECDCGDKTAWNHTLFCKAEEG

SEQ ID NO: 695 - protein, 83 aa, synthetic construct
MHSKFSHAGRICGAKFKVGEPIYRCRECSFDDTCVLCVNCFNPKDHLGHHVYTTICTEFNNGECDCGDKTAWNHTLFCKAEEG

SEQ ID NO: 696 - protein, 83 aa, synthetic construct
MHSKFSHAGRICGAKFKVGEPIYRCKLCSFDDTCVLCVNCFNPKDHLGHHVYTTICTEFNNGECDCGDKTAWNHTLFCKAEEG

SEQ ID NO: 697 - protein, 83 aa, synthetic construct
MHSKFSHAGRICGAKFKVGEPIYRCKECSFDATCVLCVNCFNPKDHLGHHVYTTICTEFNNGECDCGDKTAWNHTLFCKAEEG

SEQ ID NO: 698 - protein, 83 aa, synthetic construct
MHSKFSHAGRICGAKFKVGEPIYRCKECSFDCTCVLCVNCFNPKDHLGHHVYTTICTEFNNGECDCGDKTAWNHTLFCKAEEG

SEQ ID NO: 699 - protein, 83 aa, synthetic construct
MHSKFSHAGRICGAKFKVGEPIYRCKECSFDGTCVLCVNCFNPKDHLGHHVYTTICTEFNNGECDCGDKTAWNHTLFCKAEEG

SEQ ID NO: 700 - protein, 83 aa, synthetic construct
MHSKFSHAGRICGAKFKVGEPIYRCKECSFDHTCVLCVNCFNPKDHLGHHVYTTICTEFNNGECDCGDKTAWNHTLFCKAEEG

SEQ ID NO: 701 - protein, 83 aa, synthetic construct
MHSKFSHAGRICGAKFKVGEPIYRCKECSFDKTCVLCVNCFNPKDHLGHHVYTTICTEFNNGECDCGDKTAWNHTLFCKAEEG

SEQ ID NO: 702 - protein, 83 aa, synthetic construct
MHSKFSHAGRICGAKFKVGEPIYRCKECSFDPTCVLCVNCFNPKDHLGHHVYTTICTEFNNGECDCGDKTAWNHTLFCKAEEG

SEQ ID NO: 703 - protein, 83 aa, synthetic construct
MHSKFSHAGRICGAKFKVGEPIYRCKECSFDQTCVLCVNCFNPKDHLGHHVYTTICTEFNNGECDCGDKTAWNHTLFCKAEEG

SEQ ID NO: 704 - protein, 83 aa, synthetic construct
MHSKFSHAGRICGAKFKVGEPIYRCKECSFDRTCVLCVNCFNPKDHLGHHVYTTICTEFNNGECDCGDKTAWNHTLFCKAEEG

SEQ ID NO: 705 - protein, 83 aa, synthetic construct
MHSKFSHAGRICGAKFKVGEPIYRCKECSFDSTCVLCVNCFNPKDHLGHHVYTTICTEFNNGECDCGDKTAWNHTLFCKAEEG

SEQ ID NO: 706 - protein, 83 aa, synthetic construct
MHSKFSHAGRICGAKFKVGEPIYRCKECSFDVTCVLCVNCFNPKDHLGHHVYTTICTEFNNGECDCGDKTAWNHTLFCKAEEG

SEQ ID NO: 707 - protein, 83 aa, synthetic construct
MHSKFSHAGRICGAKFKVGEPIYRCKECSFDDTCVLCVNCFNPKDHIGHHVYTTICTEFNNGECDCGDKTAWNHTLFCKAEEG

SEQ ID NO: 708 - protein, 83 aa, synthetic construct
MHSKFSHAGRICGAKFKVGEPIYRCKECSFDDTCVLCVNCFNPKDHLGHHVYTTIRTEFNNGECDCGDKTAWNHTLFCKAEEG

SEQ ID NO: 709 - protein, 83 aa, synthetic construct
MHSKFSHAGRICGAKFKVGEPIYRCKECSFDDTCVLCVNCFNPKDHLGHHVYTTICTCFNNGECDCGDKTAWNHTLFCKAEEG

SEQ ID NO: 710 - protein, 83 aa, synthetic construct
MHSKFSHAGRICGAKFKVGEPIYRCKECSFDDTCVLCVNCFNPKDHLGHHVYTTICTDFNNGECDCGDKTAWNHTLFCKAEEG

SEQ ID NO: 711 - protein, 83 aa, synthetic construct
MHSKFSHAGRICGAKFKVGEPIYRCKECSFDDTCVLCVNCFNPKDHLGHHVYTTICTIFNNGECDCGDKTAWNHTLFCKAEEG

SEQ ID NO: 712 - protein, 83 aa, synthetic construct
MHSKFSHAGRICGAKFKVGEPIYRCKECSFDDTCVLCVNCFNPKDHLGHHVYTTICTNFNNGECDCGDKTAWNHTLFCKAEEG

SEQ ID NO: 713 - protein, 83 aa, synthetic construct
MHSKFSHAGRICGAKFKVGEPIYRCKECSFDDTCVLCVNCFNPKDHLGHHVYTTICTQFNNGECDCGDKTAWNHTLFCKAEEG

SEQ ID NO: 714 - protein, 83 aa, synthetic construct
MHSKFSHAGRICGAKFKVGEPIYRCKECSFDDTCVLCVNCFNPKDHLGHHVYTTICTSFNNGECDCGDKTAWNHTLFCKAEEG

SEQ ID NO: 715 - protein, 83 aa, synthetic construct
MHSKFSHAGRICGAKFKVGEPIYRCKECSFDDTCVLCVNCFNPKDHLGHHVYTTICTWFNNGECDCGDKTAWNHTLFCKAEEG

SEQ ID NO: 716 - protein, 83 aa, synthetic construct
MHSKFSHAGRICGAKFKVGEPIYRCKECSFDDTCVLCVNCFNPKDHLGHHVYTTICTEKNNGECDCGDKTAWNHTLFCKAEEG

SEQ ID NO: 717 - protein, 83 aa, synthetic construct
MHSKFSHAGRICGAKFKVGEPIYRCKECSFDKTCVLCVNCFNPKDHIGHHVYTTICTEFNNGECDCGDKTAWNHTLFCKAEEG

SEQ ID NO: 718 - protein, 83 aa, synthetic construct
MHSKFSHAGRICGAKFKVGEPIYRCKECSFDVTCVLCVNCFNPKDHLGHHVYTTIRTCKNNGECDCGDKTAWNHTLFCKAEEG

SEQ ID NO: 719 - protein, 83 aa, synthetic construct
MHSKFSHAGRICGAKFKVGERIYRCKECSFDDTCVLCVNCFNPKDHLGHHVYTTIRTEFNNGECDCGDKTAWNHTLFCKAEEG

SEQ ID NO: 720 - protein, 83 aa, synthetic construct
MHSKFSHAGRICGAKFKVGERIYRCKECSFDDTCVLCVNCFNPKDHLGHHVYTTIRTEKNNGECDCGDKTAWNHTLFCKAEEG

SEQ ID NO: 721 - protein, 83 aa, synthetic construct
MHSKFSHAGRICGAKFKVGEPIYRCRECSFDDTCVLCVNCFNPKDHIGHHVYTTICTEFNNGECDCGDKTAWNHTLFCKAEEG

SEQ ID NO: 722 - protein, 83 aa, synthetic construct
MHSKFSHAGRICGAKFKVGEPIYRCRECSFDVTCVLCVNCFNPKDHLGHHVYTTICTEFNNGECDCGDKTAWNHTLFCKAEEG

SEQ ID NO: 723 - protein, 83 aa, synthetic construct
MHSKFSHAGRICGAKFKVGEPIYRCRECSFDVTCVLCVNCFNPKDHIGHHVYTTICTEFNNGECDCGDKTAWNHTLFCKAEEG

SEQ ID NO: 724 - protein, 83 aa, synthetic construct; identical to SEQ ID NO: 723

SEQ ID NO: 725 - protein, 83 aa, synthetic construct
MHSKFSHAGRICGAKFKVGEPIYRCRECSFDRTCVLCVNCFNPKDHIGHHVYTTICTEFNNGECDCGDKTAWNHTLFCKAEEG

SEQ ID NO: 726 - protein, 83 aa, synthetic construct
MHSKFSHAGRICGAKFKVGEPIYRCRECSFDRTCVLCVNCFNPKDHLGHHVYTTICTQKNNGECDCGDKTAWNHTLFCKAEEG

SEQ ID NO: 727 - protein, 83 aa, synthetic construct
MHSKFSHAGRICGAKFKVGEPIYRCRECSFDHTCVLCVNCFNPKDHLGHHVYTTICTEFNNGECDCGDKTAWNHTLFCKAEEG

SEQ ID NO: 728 - protein, 83 aa, synthetic construct
MHSKFSHAGRICGAKFKVGEPIYRCRECSFDHTCVLCVNCFNPKDHIGHHVYTTICTEFNNGECDCGDKTAWNHTLFCKAEEG

SEQ ID NO: 729 - protein, 83 aa, synthetic construct
MHSKFSHAGRICGAKFKVGEPIYRCRECSFDQTCVLCVNCFNPKDHLGHHVYTTICTEKNNGECDCGDKTAWNHTLFCKAEEG

SEQ ID NO: 730 - protein, 83 aa, synthetic construct
MHSKFSHAGRICGAKFKVGEPIYRCRLCSFDVTCVLCVNCFNPKDHLGHHVYTTIRTEFNNGECDCGDKTAWNHTLFCKAEEG

SEQ ID NO: 731 - protein, 83 aa, synthetic construct
MHSKFSHAGRICGAKFKVGEPIYRCRLCSFDVTCVLCVNCFNPKDHLGHHVYTTIRTEKNNGECDCGDKTAWNHTLFCKAEEG

SEQ ID NO: 732 - protein, 83 aa, synthetic construct
MHSKFSHAGRICGAKFKVGEPIYRCRLCSFDQTCVLCVNCFNPKDHLGHHVYTTIRTSFNNGECDCGDKTAWNHTLFCKAEEG

SEQ ID NO: 733 - protein, 83 aa, synthetic construct
MHSKFSHAGRICGAKFKVGEPIYRCKLCSFDVTCVLCVNCFNPKDHLGHHVYTTIRTSKNNGECDCGDKTAWNHTLFCKAEEG

SEQ ID NO: 734 - protein, 83 aa, synthetic construct
MHSKFSHAGRICGAKFKVGEPIYRCKECSFDVTCVLCVNCFNPKDHLGHHVYTTIRTEFNNGECDCGDKTAWNHTLFCKAEEG

SEQ ID NO: 735 - protein, 83 aa, synthetic construct
MHSKFSHAGRICGAKFKVGEPIYRCKECSFDDTCVLCVNCFNPKDHLGHHVYTTIRTEKNNGECDCGDKTAWNHTLFCKAEEG

SEQ ID NO: 736 - protein, 83 aa, synthetic construct
MHSKFSHAGRICGAKFKVGEPIYRCKLCSFDDTCVLCVNCFNPKDHLGHHVYTTIRTEFNNGECDCGDKTAWNHTLFCKAEEG

SEQ ID NO: 737 - protein, 83 aa, synthetic construct
MHSKFSHAGRICGAKFKVGEPIYRCRECSFDQTCVLCVNCFNPKDHLGHHVYTTICTEFNNGECDCGDKTAWNHTLFCKAEEG

SEQ ID NO: 738 - protein, 83 aa, synthetic construct
MHSKFSHAGRICGAKFKVGEPIYRCKECSFDVTCVLCVNCFNPKDHLGHHVYTTIRTEKNNGECDCGDKTAWNHTLFCKAEEG

SEQ ID NO: 739 - protein, 83 aa, synthetic construct
MHSKFSHAGRICGAKFKVGEPIYRCRLCSFDDTCVLCVNCFNPKDHLGHHVYTTIRTEFNNGECDCGDKTAWNHTLFCKAEEG

SEQ ID NO: 740 - protein, 83 aa, synthetic construct
MHSKFSHAGRICGAKFKVGEPIYRCKLCSFDVTCVLCVNCFNPKDHLGHHVYTTIRTEFNNGECDCGDKTAWNHTLFCKAEEG

SEQ ID NO: 741 - protein, 83 aa, synthetic construct
MHSKFSHAGRICGAKFKVGEPIYRCRECSFDHTCVLCVNCFNPKDHLGHHVYTTIRTDKNNGECDCGDKTAWNHTLFCKAEEG

SEQ ID NO: 742 - protein, 115 aa, synthetic construct
MGLENSLETLRFSISNLSMQTHAARMRTFMYWPSSVPVQPEQLASAGFYYLGRNDDVCCFCCDGALRCWQSGDDPWVEHALWFPRCEFLIRMKGQEFVDEIQGRYPHLLEQLLSG

SEQ ID NO: 743 - protein, 115 aa, synthetic construct
MGLENSLETLRFSISNLSMQTHAARMRTFMYWPSSVPVQPEQLASAGFYYVGRNDIVRCFCCDGALWCWESGDDPWVEHAKWFPRCEFLIRMKGQEFVDEIQGRYPHLLEQLLSG

SEQ ID NO: 744 - protein, 115 aa, synthetic construct
MGLENSLETLRFSISNLSMQTHAARMRTFMYWPSSVPVQPEQLASAGFYYLGRNDEVRCFCCDGGLHCWQSGDDPWVEHALWFPRCEFLIRMKGQEFVDEIQGRYPHLLEQLLSG

SEQ ID NO: 745 - protein, 115 aa, synthetic construct
MGLENSLETLRFSISNLSMQTHAARMRTFMYWPSSVPVQPEQLASAGFYYSGRNDEVRCFCCDGVLHCWESGDDPWVEHAKHFPRCEFLIRMKGQEFVDEIQGRYPHLLEQLLSG

SEQ ID NO: 746 - protein, 115 aa, synthetic construct
MGLENSLETLRFSISNLSMQTHAARMRTFMYWPSSVPVQPEQLASAGFYYSGRNDLVACFCCDGGLTCWESGDDPWVEHAKHFPRCEFLIRMKGQEFVDEIQGRYPHLLEQLLSG

SEQ ID NO: 747 - protein, 115 aa, synthetic construct
MGLENSLETLRFSISNLSMQTHAARMRTFMYWPSSVPVQPEQLASAGFYYLGRNDIVRCFCCDGVLGCWESGDDPWVEHARHFPRCEFLIRMKGQEFVDEIQGRYPHLLEQLLSG

SEQ ID NO: 748 - protein, 115 aa, synthetic construct
MGLENSLETLRFSISNLSMQTHAARMRTFMYWPSSVPVQPEQLASAGFYYLGRNDEVRCFCCDGGLHCWQSGDDPWVEHARWFPRCEFLIRMKGQEFVDEIQGRYPHLLEQLLSG

SEQ ID NO: 749 - protein, 115 aa, synthetic construct
MGLENSLETLRFSISNLSMQTHAARMRTFMYWPSSVPVQPEQLASAGFYYVGRNDIVRCFCCDGALHCWKSGDDPWVEHARHFPRCEFLIRMKGQEFVDEIQGRYPHLLEQLLSG

SEQ ID NO: 750 - protein, 115 aa, synthetic construct
MGLENSLETLRFSISNLSMQTHAARMRTKMYWESSVPVQWEQLASYGFQFVGRNDDVKCQCCDGGLRCWESGDDVAVEHSKRFIRCEFLIRMKGQEFVDEIQGRYPHLLEQLLSG

SEQ ID NO: 751 - protein, 115 aa, synthetic construct
MGLENSLETLRFSISNLSMQTHAARMRTFMYWPSSVPVQPEQLASAGFYYLGRNDLVKCFCCDGVLHCWQSGDDPWVEHARHFPRCEFLIRMKGQEFVDEIQGRYPHLLEQLLSG

SEQ ID NO: 752 - protein, 115 aa, synthetic construct
MGLENSLETLRFSISNLSMQTHAARMRTFMYWPSSVPVQPEQLASAGFYYLGRNDIVKCFCCDGVLHCWQSGDDPWVEHAKHFPRCEFLIRMKGQEFVDEIQGRYPHLLEQLLSG

SEQ ID NO: 753 - protein, 115 aa, synthetic construct
MGLENSLETLRFSISNLSMQTHAARMRTFMYWPSSVPVQPEQLASAGFYYLGRNDEVRCFCCDGVLHCWESGDDPWVEHARWFPRCEFLIRMNGQEFVDEIQGRYPHLLEQLLSG

SEQ ID NO: 754 - protein, 115 aa, synthetic construct
MGLENSLETLRFSISNLSMQTHAARMRTFMYWPSSVPVQPEQLASAGFYYSGRNDIVRCFCCDGDLHCWESGDDPWVEHAKWFPRCEFLIRMKGQEFVDEIQGRYPHLLEQLLSG

SEQ ID NO: 755 - protein, 115 aa, synthetic construct
MGLENSLETLRFSISNLSMQTHAARMRTFMYWPSSVPVQPEQLASAGFYYAGRNDEVKCFCCDGGLHCWESGDDPWVEHARHFPRCEFLIRMNGQEFVDEIQGRYPHLLEQLLSG

SEQ ID NO: 756 - protein, 115 aa, synthetic construct
MGLENSLETLRFSISNLSMQTHAARMRTFMYWPSSVPVQPEQLASAGFYYLGRNDIVKCFCCDGVLHCWQSGDDPWVEHARHFPRCEFLIRMKGQEFVDEIQGRYPHLLEQLLSG

SEQ ID NO: 757 - protein, 115 aa, synthetic construct
MGLENSLETLRFSISNLSMQTHAARMRTFMYWPSSVPVQPEQLASAGFYYLGRNDLVKCFCCDGVLHCWQSGDDPWVEHAKHFPRCEFLIRMKGQEFVDEIQGRYPHLLEQLLSG

SEQ ID NO: 758 - protein, 115 aa, synthetic construct
MGLENSLETLRFSISNLSMQTHAARMRTFMYWPSSVPVQPEQLASAGFYYLGRNDLVKCFCCDGVLHCWQSGDDPWVEHAKWFPRCEFLIRMKGQEFVDEIQGRYPHLLEQLLSG

SEQ ID NO: 759 - protein, 115 aa, synthetic construct
MGLENSLETLRFSISNLSMQTHAARMRTFMYWPSSVPVQPEQLASAGFYYLGRNDLVKCFCCDGVLHCWESGDDPWVEHARHFPRCEFLIRMKGQEFVDEIQGRYPHLLEQLLSG

SEQ ID NO: 760 - protein, 115 aa, synthetic construct
MGLENSLETLRFSISNLSMQTHAARMRTFMYWPSSVPVQPEQLASAGFYYLGRNDIVKCFCCDGVLHCWQSGDDPWVEHAKWFPRCEFLIRMKGQEFVDEIQGRYPHLLEQLLSG

SEQ ID NO: 761 - protein, 115 aa, synthetic construct
MGLENSLETLRFSISNLSMQTHAARMRTFMYWPSSVPVQPEQLASAGFYYLGRNDLVKCFCCDGALHCWQSGDDPWVEHARHFPRCEFLIRMKGQEFVDEIQGRYPHLLEQLLSG

SEQ ID NO: 762 - protein, 115 aa, synthetic construct
MGLENSLETLRFSISNLSMQTHAARMRTFMYWPSSVPVQPEQLASAGFYYLGRNDLVKCFCCDGVLHCWQSGDDPWVEHARHFPRCEFLIRMKGQEFVDEVQGRYPHLLEQLLSG

SEQ ID NO: 763 - protein, 115 aa, synthetic construct
MGLENSLETLRFSISNLSMQTHAARMRTFMYWPSSVPVQPEQLASAGFYYLGRNDLVKCFCCDGVLHCWQSGDDPWVEHARHFPRCEFLIRMKGQEFVDEIQGRYPHLLEQLPSG

SEQ ID NO: 764 - protein, 115 aa, synthetic construct
MGLENSLETLRFSISNLSMQTHAARMRTFMYWPSSVPVQPEQLASAGFYYLGRNDLVKCFCCDGVLRCWESGDDPWVEHAKHFPRCEFLIRMKGQEFVDEIQGRYPHLLEQLLSG

SEQ ID NO: 765 - protein, 115 aa, synthetic construct
MGLENSLETLRFSISNLSMQTHAARMRTFMYWPSSVPVQPEQLASAGFYYLGRNDLVKCFCCDGVLHCWQSGDDPWVEHATWFPRCEFLIRMKGQEFVDEIQGRYPHLLEQLLSG

SEQ ID NO: 766 - protein, 115 aa, synthetic construct
MGLENSLETLRFSISNLSMQTHAARMRTFMYWPSSVPVQPEQLASAGFYYGGRNDLVKCFCCDGVLHCWQSGDDPWVEHARHFPRCEFLIRMKGQEFVDEIQGRYPHLLEQLLSG

SEQ ID NO: 767 - protein, 115 aa, synthetic construct
MGLENSLETLRFSISNLSMQTHAARMRTFMYWPSSVPVQPEQLASAGFYYLGRNDLVKCFCCDGVLHCWQGGDDPWVEHARHFPRCEFLIRMKGQEFVDEIQGRYPHLLEQLLSG

SEQ ID NO: 768 - protein, 119 aa, synthetic construct
MPTAASATESAIEDTPAPARPEVDGRTKPKRQPRYHVVLWDDDDHTYAYVVVMLVSLFGHPPSRGYRMAKEMDVQGRVIVLTTTRAHAELKRDQIHAFGRDRLLARSKGSMKASIEAEE

SEQ ID NO: 769 - protein, 119 aa, synthetic construct
MPTAASATESAIEDTPAPARPEVDGRTKPKRQPRYHVVLWDDDDHTYAYVVVMLRSLFGHPPSRGYRMAKEMDVQGRVIVLTTTRAHAELKRDQIHAFGRDRLLARSKGSMKASIEAEE

SEQ ID NO: 770 - protein, 119 aa, synthetic construct
MPTAASATESAIEDTPAPARPEVDGRTKPKRQPRYHVVLWDDDDHTYQYVVTMLRSLFGHPPSRGYRMAKEMDTQGRVIVLTTTREHAELKRDQIHAFGRDRLLARSKGSMKASIEAEE

SEQ ID NO: 771 - protein, 119 aa, synthetic construct
MPTAASATESAIEDTPAPARPEVDGRTKPKRQPRYHVVLWDDDDHTYQYVVVMLRSLFGHPPGRGYRMAKEMDTQGRVIVLTTTREHAELKRDQIHAFGRDRLLARSKGSMKASIEAEE

SEQ ID NO: 772 - protein, 119 aa, synthetic construct
MPTAASATESAIEDTPAPARPEVDGRTKPKRQPRYHVVLWDDDDHTYQYVVVMLRSLFGHPPSRGYRIAKEMDTQGRVIVLTTTREHAELKRDQIHAFGRDRLLARSKGSMKASIEAEE

SEQ ID NO: 773 - protein, 119 aa, synthetic construct
MPTAASATESAIEDTPAPARPEVDGRTKPKRQPRYHVVLWDDDDHTYQYVVVMLRSLFGHPPSRGYRMAKEVDTQGRVIVLTTTREHAELKRDQIHAFGRDRLLARSKGSMKASIEAEE

SEQ ID NO: 774 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAEFKRDQIHAFGRDRLLARSKGSMKASIEAEE

SEQ ID NO: 775 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQINAFGRDRLLARSKGSMKASIEAEE

SEQ ID NO: 776 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDSLLARSKGSMKASIEAEE

SEQ ID NO: 777 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDGLLARSKGSMKASIEAEE

SEQ ID NO: 778 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGRDRLLARSKGSMTASIEAEE

SEQ ID NO: 779 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAWGRDSLLARSKGSMKASIEAEE

SEQ ID NO: 780 - protein, 119 aa, synthetic construct
MPTAASATESAIEDTPAPARSEVDGYTVPKRQPRYHVVLWDDDDHTYQYVVGMLRSVFGHPPSRGYRMAKEMDTQGRVIVLTTTREHAELKRDQIHAFGRDRLLARSKGSMKASIEAEE

SEQ ID NO: 781 - protein, 119 aa, synthetic construct
MPTAASATESAIEDTPAPVRPEVDGYTVPKRQPRYHVVLWDDDDHTYQYVVTMLRSLFGHPPGRGYRMAKEMDTQGRVIVLTTTREHAELKRDQIHAFGRDRLLARSKGSMKASIEAEE

SEQ ID NO: 782 - protein, 119 aa, synthetic construct
MPTAASATESAMEDTPAPARPEVDGRTKPKRQPRYHVVLWNDDDHTYQYVVVMLQSLFGHPPKRGYRMAKEMDTQGRVIVLTTTREHAELKRDQIHAFGRDRLLARCKGSMKASIEAEE

SEQ ID NO: 783 - protein, 119 aa, synthetic construct
MPTAASATESAIEDTPAPARPEVDGRTKPKRQPRYHVVLWDDDDHTYQYVVVMLQSLFGHPPNRGYRMAKEMDTQGRVIVLTTTREHAELKRDQIHAFGCDRLLARCKGSMKASIEAEE

SEQ ID NO: 784 - protein, 119 aa, synthetic construct
MPTAASATESAIEDPPAPARPEVDGRTKPKRQPRYHVVMWEDDDHTYQYVVVMLRSLFGHPPNRGYRMAKEMDTQGRVIVLTTTREHAELKRDQIHAFGCDRLLARSKGSMKASIEAEE

SEQ ID NO: 785 - protein, 119 aa, synthetic construct
MPTAASATESAIEDTPAPARPEVDGRTKPKRQPRYHVVLWDDDDHTYQYVVVMLRSLFGHPPSRGYRMAKELDTQGRVIVLTTTREHAELKRDQIRAYGRDGLLARSKGSMKASIEAEE

SEQ ID NO: 786 - protein, 119 aa, synthetic construct
MPTAASATESAIEDTPAPARSEVDGRTEPKRQPRYHVVLWDDDDHTYQYVVAMLRSLFGHPPSRGYRMAKEMDTQGRVIVLTTTREHAELKRDQIHAFGRDSLLARSKGSMKASIEAEE

SEQ ID NO: 787 - protein, 119 aa, synthetic construct
MPTAASATESAIEDTPAPARPEVDGRTKPKRQPRYHVVLWDDDDHTYQYVVVMLRSLFGHSASRGYRMAKEMDTQGRVIVLTTTREHAELKRDQIHAFGRDRLLARSKGSMKASIEAEE

SEQ ID NO: 788 - protein, 119 aa, synthetic construct
MPTAASATESAIEDTPAPARSEVDGYTVPKRQPRYHVVLWDDDDHTYQYVVVMLRSLFGHPPSRGYRMAKEMDTQGRVIVLTTTREHAELKRDQIHAFGRDRLLARSKGSMKASIEAEE

SEQ ID NO: 789 - protein, 119 aa, synthetic construct
MPTAASATESAIEDTPAPARPEVDGRTKPKRQPRYHVVLWDDDDHTYQYVVHMLRSIFGHPPSRGYRMAKEMDTQGRVIVLTTTREYAELKRDQIHAFGRDRLLARSKGSMKASIEAEE

SEQ ID NO: 790 - protein, 119 aa, synthetic construct
MPTAASATESAIEDTPAPARPEVDGRTKPKRQPRYHVVLWDDDDHTYQYVVVMLRSLFGHPPERGYRMAKEMDTQGRVIVLTTTREHAELKRDQIHAFGRDRLLARSKGSMKASIEAEE

SEQ ID NO: 791 - protein, 119 aa, synthetic construct
MPTAASATESAIEDTPAPARPEVDGRTKPKRQPRYHVVLWDDDDHTYQYVVVMLRSLFGHPPSRGYRMAKEMDTQGRVIVLTTTREHAELKRDQIHAFGYDRLLARSKGSMKASIEAEE

SEQ ID NO: 792 - protein, 200 aa, synthetic construct
MNGLSAQHERIAPARHECVYTSCYSEENIWKLCEHIKTSKRCPLGDVYAVFISNERKMVPIWKLKSGRGEEPVIWDYNVILLHDCHKEQTFIYDLDTTLPFPCPFDTYVKEAFKSDNYINPAFWRKLRVVPADVFLQNFASDRSHNKDASGGWRMPPPPYPCIETAESRMNLDDFISMNPSVGWGHVYTLEEFVQHFGKT

SEQ ID NO: 793 - protein, 200 aa, synthetic construct
MNGLSAQHERIAPARHECVYTSCYSEENVWKLCEHIKTSKRCPLGDVYAVFISNERKMVPIWKKKSGRGEELLIWDYNVIILHDCHKEQTFIYDLDTTLPFPCPFDTYVKEAFKSDNYINPAFWRKLRVVPADVFLQNFASDRSHQKDASGGWRMPPPPYPCIETAESRMNLDDFISMNPSVGWGHVYTLEEFVQHFGKT

SEQ ID NO: 794 - protein, 200 aa, synthetic construct
MNGLSAQHERIAPARHECVYTSCYAEENVWKVCEHIKTSKRCPLGDVYAVFISNERKMVPIWKRKSGRGEEFIIWDYQVIFLHDCHKEQTFIYDLDTTLPFPCPFDTYVKEAFKSDNYINPAFWRKLRVVPADVFLQNFASDRSHNKDASGGWRMPPPPYPCIETAESRMNLDDFISMNPSVGWGHVYTLEEFVQHFGKT

SEQ ID NO: 795 - protein, 200 aa, synthetic construct
MNGLSAQHERIAPARHECVYTSCYAEENAWKFCEHIKTSKRCPLGDVYAVFISNERKMVPIWKKKSGRGEEPVIWDYQVIVLHDCHKEQTFIYDLDTTLPFPYPFDTYVKEAFKSDNYINPAFWRKLRVVPADVFLQNFASDRSHNKDASGGWRMPPPPYPCIETAESRMNLDDFISMNPSVGWGHVYTLEEFVQHFGKT

SEQ ID NO: 796 - protein, 200 aa, synthetic construct
MNGLSAQHERIAPARHECVYTSCYAEENAWKMCEHIKTSKKCPLGDVYAVFISNERKMVPIWKKKSGQGEEFLIWDYNVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 797 - protein, 200 aa, synthetic construct
MNGLSAQHERIAPARHECVYTSCYAEENLFKICEHIKTSKRCPLGDVYAVFISNERKMVPIWKPKSGRGEEVIIWDYNVIVLHDCHKEQTFIYDLDTTLPFPCPFDTYVKEAFKSDNYINPAFWRKLRVVPADVFLQNFASDRSHNKDASGGWRMPPPPYPCIETAESRMNLDDFISMNPSVGWGHVYTLEEFVQHFGKT

SEQ ID NO: 798 - protein, 200 aa, synthetic construct
MNGLSAQHERIAPARHECVYTSCYAEENLFKICEHIKTSKRCPLGDVYAVFISNERKMVPIWKPKSGRGEEVIIWDYNVIVLHDCHKEQTFIYDLDTTLPFPCPFDTYVKEAFKSDNYINHAFWRKLRVVPADVFLQNFASDRSHNKDASGGWRMPPPPYPCIETAESRMNLDDFISMNPSVGWGHVYTLEEFVQHFGKT

SEQ ID NO: 799 - protein, 200 aa, synthetic construct
MNGLSAQHERIAPARHECVYTSCYSEENVWKLCEHIKTSKRCPLGDVYAVFISNERKMVPIWKLKSGQGEEPVIWDYQVILLHDCHKEQTFIYDLDTTLPFPCPFDTYVKEAFKSDNYINHAFWRKLRVVPADVFLQNFASDRSHNKDASGGWRMPPPPYPCIETAESRMNLDDFISMNPSVGWGHVYTLEEFVQHFGKT

SEQ ID NO: 800 - protein, 200 aa, synthetic construct
MNGLSAQHERIAPARHECVYTSCYSEENVWKLCEHIKTSKRCPLGDVYAVFISNERKMVPIWKLKSGQGEEPVIWDYQVILLHDCHKEQTFIYDLDTTLPFPCPFDTYVKEAFKSDNYINHAFWRKLRVVPADVFLQNFASDRSHQKDASGGWRMPPPPYPCIETAESRMNLDDFISMNPSVGWGHVYTLEEFVQHFGKT

SEQ ID NO: 801 - protein, 200 aa, synthetic construct
MNGLSAQHERIAPARHECVYTSCYAEENAWKFCEHIKTSKRCPLGDVYAVFISNERKMVPIWKKKSGRGEEPVIWDYQVIVLHDCHKEQTFIYDLDTTLPFPYPFDTYVKEAFKSDNYINPAFWRKLRVVPADVFLQNFASDRSHQKDASGGWRMPPPPYPCIETAESRMNLDDFISMNPSVGWGHVYTLEEFVQHFGKT

SEQ ID NO: 802 - protein, 200 aa, synthetic construct
MNGLSAQHERIAPARHECVYTSCYSEENASKLCEHIKTSKRCPLGDVYAVFISNERKMVPIWKQKSGRGEEPVIWDYHVIILHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 803 - protein, 200 aa, synthetic construct
MNGLSAQHERIAPARHECVYTSCYAEENLFKICEHIKTSKRCPLGDVYAVFISNERKMVPIWKPKSGRGEEVIIWDYNVIVLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 804 - protein, 200 aa, synthetic construct
MNGLSAQHERIAPARHECVYTSCYSEENVWKACEHIKTSKRCPLGDVYAVFISNERKMVPIWKKKSGRGEEPVIWDYHVIVLHDCHKEQTFIYDLDTTLPFPCPFDTYVKEAFKSDNYINPAFWRKLRVVPADVFLQNFASDRSHNKDASGGWRMPPPPYPCIETAESRMNLDDFISMNPSVGWGHVYTLEEFVQHFGKT

SEQ ID NO: 805 - protein, 200 aa, synthetic construct
MNGLSAQHERIAPARHECVYTSCYAEENAWKFCEHIKTSKRCPLGDVYAVFISNERKMVPIWKPKSGRGEEVIIWDYNVIVLHDCHKEQTFIYDLDTTLPFPCPFDTYVKEAFKSDNYINPAFWRKLRVVPADVFLQNFASDRSHQKDASGGWRMPPPPYPCIETAESRMNLDDFISMNPSVGWGHVYTLEEFVQHFGKT

SEQ ID NO: 806 - protein, 200 aa, synthetic construct
MNGLSAQHERIAPARHECVYTSCYAEENLFKICEHIKTSKRCPLGDVYAVFISNERKMVPIWKLKSGQGEEPVIWDYQVILLHDCHKEQTFIYDLDTTLPFPCPFDTYVKEAFKSDNYINHAFWRKLRVVPADVFLQNFASDRSHNKDASGGWRMPPPPYPCIETAESRMNLDDFISMNPSVGWGHVYTLEEFVQHFGKT

SEQ ID NO: 807 - protein, 200 aa, synthetic construct
MNGLSAQHERIAPARHECVYTSCYSEENVWKLCEHIKTSKRCPLGDVYAVFISNERKMVPIWKLKSGQGEEPVIWDYQVILLHDCHKEQPFIYDLDTTLPFPCPFDTYVKEAFKSDNYINHAFWRKLRVVPADVFLQNFASDRSHNKDASGGWRMPPPPYPCIETAESRMNLDDFISMNPSVGWGHVYTLEEFVQHFGKT

SEQ ID NO: 808 - protein, 200 aa, synthetic construct
MNGLSAQHERIAPARHECVYTSCYAEENLWKACEHIKTSKRCPLGDVYAVFISNERKMVPIWKKKSGRGEEFIIWDYNVIVLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 809 - protein, 200 aa, synthetic construct
MNGLSAQHERIAPARHECVYTSCYAEENLFKICEHIKTSKRCPLGDVYAVFISNERKMVPIWKPKSGRGEEVIIWDYNVIVLHDCHKEQTFNYDLDTTLPFPCPFDTYVKEAFKSDNYINPAFWRKLRVVPADVFLQNFASDRSHQKDASGGWRMPPPPYPCIETAESRMNLDDFISMNPSVGWGHVYTLEEFVQHFGKT

SEQ ID NO: 810 - protein, 200 aa, synthetic construct
MNGLSAQHERIAPARHECVYTSCYAEENAWKFCEHIKTSKRCPLGDVYAVFISNERKMVPIWKKKSGRGEEPVIWDYQVIVLHDCHKEQPFIYDLDTTLPFPYPFDTYVKEAFKSDNYINPAFWRKLRVVPADVFLQNFASDRSHNKDASGGWRMPPPPYPCIETAESRMNLDDFISMNPSVGWGHVYTLEEFVQHFGKT

SEQ ID NO: 811 - protein, 200 aa, synthetic construct
MNGLSAQHERIAPARHECVYTSCYAEENLFKICEHIKTSKRCPLGDVYAVFISNERKMVPIWKPKSGRGEEVIIWDYNVIVLHDCHKEQPFIYDLDTTLPFPCPFDTYVKEAFKSDNYINPAFWRKLRVVPADVFLQNFASDRSHQKDASGGWRMPPPPYPCIETAESRMNLDDFISMNPSVGWGHVYTLEEFVQHFGKT

SEQ ID NO: 812 - protein, 200 aa, synthetic construct
MNGLSAQHERIAPARHECVYTSCYAEENAWKFCEHIKTSKRCPLGDVYAVFISNERKMVPIWKLKSGQGEEPVIWDYQVILLHDCHKEQTFIYDLDTTLPFPCPFDTYVKEAFKSDNYINHAFWRKLRVVPADVFLQNFASDRSHNKDASGGWRMPPPPYPCIETAESRMNLDDFISMNPSVGWGHVYTLEEFVQHFGKT

SEQ ID NO: 813 - protein, 200 aa, synthetic construct
MNGLSAQHERIAPARHECVYTSCYAEENAWKFCEHIKTSKRCPLGDVYAVFISNERKMVPIWKRKSGRGEEPVIWDYQVIVLHDCHKEQTFIYDLDTTLPFPCPFDTYVKEAFKSDNYINPAFWRKLRVVPADVFLQNFASDRSHQKDASGGWRMPPPPYPCIETAESRMNLDDFISMNPSVGWGHVYTLEEFVQHFGKT

SEQ ID NO: 814 - protein, 200 aa, synthetic construct
MNGLSAQHERIAPARHECVYTSCYAEENLFKICEHIKTSKRCPLGDVYAVFISNERKMVPIWKPKSGRGEEVIIWDYNVIVLHDCHKEQTFIYDLDTTLPFPCPFDTYVKEAFKSDNYINPAFWRKLRVVPADVFLQNFASDRSHQKDASGGWRMPPPPYPCIETAESRMNLDDFISMNPSVGWGHVYTLEEFVQHFGKT

SEQ ID NO: 815 - protein, 200 aa, synthetic construct
MNGLSAQHERIAPARHECVYTSCYSEENVWKLCEHIKTSKRCPLGDVYAVFISNERKMVPIWKRKSGRGEEPVIWDYQVILLHDCHKEQTFIYDLDTTLPFPCPFDTYVKEAFKSDNYINPAFWRKLRVVPADVFLQNFASDRSHNKDASGGWRMPPPPYPCIETAESRMNLDDFISMNPSVGWGHVYTLEEFVQHFGKT

SEQ ID NO: 816 - protein, 200 aa, synthetic construct
MNGLSAQHERIAPARHECVYTSCYSEENVWKLCEHIKTSKRCPLGDVYAVFISNERKMVPIWKKKSGRGEELLIWDYNVIILHDCHKEQTFIYDLDTTLPFPCPFDTYVKEAFKSDNYINPAFWRKLRVVPADVFLQNFASDRSHNKDASGGWRMPPPPYPCIETAESRMNLDDFISMNPSVGWGHVYTLEEFVQHFGKT

SEQ ID NO: 817 - protein, 200 aa, synthetic construct
MNGLSAQHERIAPARHECVYTSCYAEENVFKACEHIKTSKSCPLGDVYAVFISNERKMVPIWKQKSGRSEEFIIWDYQVIILHDCHKEQTFIYDLDTTLPFPCPFDTYVKEAFKSDNYINPAFWRKLRVVPADVFLQNFASDRSHQKDASGGWRMPPPPYPCIETAESRMNLDDFISMNPSVGWGHVYTLEEFVQHFGKT

SEQ ID NO: 818 - protein, 200 aa, synthetic construct
MNGLSAQHERIAPARHECVYTSCYAEENLWKLCEHIKTSKRCPLGDVYAVFISNERKMVPIWKPKSGRGEELWIWDYQVIILHDCHKEQTFIYDLDTTLPFPCPFDTYVKEAFKSDNYINPAFWRKLRVVPADVFLQNFASDRSHNKDASGGWRMPPPPYPCIETAESRMNLDDFISMNPSVGWGHVYTLEEFVQHFGKT

SEQ ID NO: 819 - protein, 200 aa, synthetic construct
MNGLSAQHERIAPARHECVYTSCYSEENVWKLCEHIKTSKRCPLGDVYAVFISNERKMVPIWKPKSGRGEEVIIWDYNVIVLHDCHKEQTFIYDLDTTLPFPCPFDTYVKEAFKSDNYINPAFWRKLRVVPADVFLQNFASDRSHQKDASGGWRMPPPPYPCIETAESRMNLDDFISMNPSVGWGHVYTLEEFVQHFGKT

SEQ ID NO: 820 - protein, 200 aa, synthetic construct
MNGLSAQHERIAPARHECVYTSCYSEENVWKLCEHIKTSKRCPLGDVYAVFISNERKMVPIWKLKSGQGEEPVIWDYQVILLHDCHKEQTFIYDLDTTLPFPCPFDTYVKEAFKSDNYINPAFWRKLRVVPADVFLQNFASDRSHNKDASGGWRMPPPPYPCIETAESRMNLDDFISMNPSVGWGHVYTLEEFVQHFGKT

SEQ ID NO: 821 - protein, 200 aa, synthetic construct
MNGLSAQHERIAPARHECVYTSCYSEENVWKLCEHIKTSKRCPLGDVYAVFISNERKMVPIWKLKSGQGEEPVIWDYQVILLHDCHKEQTFIYDLDTTLPFPCPFDTYVKEAFKSDNYINPAFWRKLRVVPADVFLQNFASDRSHQKDASGGWRMPPPPYPCIETAESRMNLDDFISMNPSVGWGHVYTLEEFVQHFGKT

SEQ ID NO: 822 - protein, 200 aa, synthetic construct
MNGLSAQHERIAPARHECVYTSCYSKENLWKLCEHIKTSKRCPLGDVYAVFISNERKMVPIWKKKSGRGEELWIWDYNVIVLHDCHKEQTFIYDLDTTLPFPCPFDTYVKEAFKSDNYINPAFWRKLRVVPADVFLQNFASDRSHTKDASGGWRMPPPPYPCIETAESRMNLDDFISMNPSVGWGHVYTLEEFVQHFGKT

SEQ ID NO: 823 - protein, 200 aa, synthetic construct
MNGLSAQHERIAPARHECVYTSCYSEENAWKMCEHIKTSKRCPLGDVYAVFISNERKMVPIWKQKSGRGEEPVIWDYNVIFLHDCHKEQTFIYDLDTALPFPCPFDTYVKEAFKSDNYINPAFGRKLRVVPADVFLQNFASDCSHQKDASGGWRMPPPPYPCIETAESRMNLDDFISMNPSVGWGHVYTLEEFVQHFGKT

SEQ ID NO: 824 - protein, 200 aa, synthetic construct
MNGLSAQHERIAPARHECVYTSCYAEENAWKFCEHIKTSKRCPLGDVYAVFISNERKMVPIWKRKSGRGEEPVIWDYQVIVLHDCHKEQTFIYDLDTTLPFPCPFDTYVKEAFKSDNCINPAFWRKLRVVPADVFLQNFASDRSHQKDASGGWRMPPPPYPCIETAESRMNLDDFISMNPSVGWGHVYTLEEFVQHFGKT

SEQ ID NO: 825 - protein, 200 aa, synthetic construct
MNGLSAQHERIAPARHECVYTSCYSEENVWKLCEHIKTSKRCPLGDVYAVFISNERKMVPIWKKKSGRGEEFLIWDYNVIVLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 826 - protein, 200 aa, synthetic construct
MNGLSAQHERIAPARHECVYTSCYSEENLFKMCEHIKTSKRCPLGDVYAVFISNERKMVPIWKRKSGRGEEPLIWDYHVIFLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 827 - protein, 200 aa, synthetic construct
MNGLSAQHERIAPARHECVYTSCYSEENAWKMCEHIKTSKRCPLGDVYAVFISNERKMVPIWKQKSGRGEEIWIWDYHVIILHDCHKEQTFIYDLDTTLPFPCPFDTYVKEAFKSDNYINPAFWRKLRVVPADVFLQNFASDRSHNKDASGGWRMPPPPYPCIETAESRMNLDDFISMNPSVGWGHVYTLEEFVQHFGKT

SEQ ID NO: 828 - protein, 200 aa, synthetic construct
MNGLSAQHERIAPARHECVYTSCYSEENVWKLCEHIKTSKRCPLGDVYAVFISNERKMVPIWKQKSGRGEEFLIWDYNVIVLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 829 - protein, 200 aa, synthetic construct
MNGLSAQHERIAPARHECVYTSCYAEENAWKMCEHIKTSKRCPLGDVYAVFISNERKMVPIWKRKSGRGEEPIIWDYHVIILHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 830 - protein, 200 aa, synthetic construct
MNGLSAQHERIAPARHECVYTSCYAEENIWKACVHIKTSKRCPLGDVYVVFISNERKMVPIWKQKSGRGEEFLIWDYNVIILHDCHKEQTLIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 831 - protein, 200 aa, synthetic construct
MNGLSAQHERIAPARHECVYTSCYSEENAWKMCEHIKTSKRCPLGDVYAVFISNERKMVPIWKQKSGRGEEPVIWDYHVIVLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 832 - protein, 200 aa, synthetic construct
MNGLSAQHERIAPARHECVYTSCYAEENIWKACVHIKTSKRCPLGDVYVVFISNERKMVPIWKQKSGRGEEFLIWDYNVIILHDCHKEQTFIYDLDTTLPFPCPFDTCVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 833 - protein, 200 aa, synthetic construct
MNGLSAQHERIAPARHECVYTSCYAEENIWKACVHIKTSKRCPLGDVYVVFISNERKMVPIWKQKSGRGEEFLIWDYNVIILHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 834 - protein, 244 aa, synthetic construct
MGLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSEAAAKEAAAKEAAAKMGLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSG

SEQ ID NO: 835 - protein, 411 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKTEEEKRKREEEEMNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 836 - protein, 200 aa, synthetic construct
MNGLSAQHERIAPARHECVYTSCYSEENVWKLCEHIKTSKRCPLGDVYAVFISNERKMVPIWKQKSGRGEEPVIWDYRVILLHDCHKEQTFIYDLDTTLPFPCPFDTYVKEAFKSDNYINPAFWRKLRVVPADVFLQNFASDRSRLKDASGGWRMPPPPYPCIETAESRMNLDDFISMNPSVGWGHVYTLEEFVQHFGKT

SEQ ID NO: 837 - protein, 200 aa, synthetic construct
MNGLSAQHERIAPARHECVYTSCYSEENVWKLCEHIKTSKRCPLGDVYAVFISNERKMVPIWKQKSGRGEEPVIKDYAVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 838 - protein, 200 aa, synthetic construct
MNGLSAQHERIAPARHECVYTSCYSEENVWKLCEHIKTSKRCPLGDVYAVFISNERKMVPIWKQKSGRGEEPVIWFYSVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 839 - protein, 200 aa, synthetic construct
MNGLSAQHERIAPARHECVYTSCYSEENVWKLCEHIKTSKRCPLGDVYAVFISNERKMVPIWKQKSGRGEEPVIIFYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 840 - protein, 200 aa, synthetic construct
MNGLSAQHERIAPARHECVYTSCYSEENVWKLCEHIKTSKRCPLGDVYAVFISNERKMVPIWKQKSGRGEEPVKWDYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 841 - protein, 200 aa, synthetic construct
MNGLSAQHERIAPARHECVYTSCYSEENVWKLCEHIKTSKRNPLGDVYAVFISNERKMVPIWKQKSGRGEEPVIWDYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 842 - protein, 200 aa, synthetic construct
MNGLSAQHERIAPARHECVYTSCYSEENVWKLCEHIKTSKRCPLGDVYAVFISNERKMVPIWKQKSGRGEEPVIWDYHVCLLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 843 - protein, 200 aa, synthetic construct
MNGLSAQHERIAPARHECVYTSCYSEENVWKLCEHIKTSKRCPLGDVYAVFISNERKMVPIWKQKSGRGEEPVIWDYHVILLHDCHKEQTFIYDNDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 844 - protein, 200 aa, synthetic construct
MNGLSAQHERIAPARHECVYTSCYSEENVWKLCEHIKTSKRCPLGDVYAVFISNERKMVPIWKQKSGRGEEPVIWDYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEQFVQHFGKT

SEQ ID NO: 845 - protein, 200 aa, synthetic construct
MNGLSAQHERIAPARHECVYTSCYSEENVWKLCEHIKTSKRCPLGDVYAVFISNERKMVPIWKQKSGRGEEPVIWDYHVILLHDCHKEQTFIYDLDTTLPFPCPFDTYVKEAFKSDNYINPAFWRKLRVVPADVFLQNFASDRSHMKDASGGWRMPPPPYPCIETGESRMNLDDFISMNPSVGWGHVYTLEEFVQHFGKT

SEQ ID NO: 846 - protein, 200 aa, synthetic construct
MNGLSAQHERIAPARHECVYTSCYSEENVWKLCEHIKTSKRCPLGDVYAVFISNERKMVPIWKQKSGRGEEPVIWDYHVILLHDCHKEQTFIYDLDTTLPFPCPFDTYVKETFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 847 - protein, 200 aa, synthetic construct
MNGLSAQHERIAPARHECVYTSCYSEENVWKLCEHIKTSKRCPLGDVYAVFISNERKMVPIWKQKSGRGEEPVIWDYHVILLHDCHKEQTFIYDLDTTLPFPCPFDTYVKETFKSDNYINPAFWRKLRVVPADVFLQNFASDRSRLKDASGGWRMPPPPYPCIETAESRMNLDDFISMNPSVGWGHVYTLEEFVQHFGKT

SEQ ID NO: 848 - protein, 200 aa, synthetic construct
MNGLSAQHERIAPARHECVYTSCYSEENVWKLCEHIKTSKRNPLGDVYAVFISNERKMVPIWKQKSGRGEEPVIWFYAVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 849 - protein, 200 aa, synthetic construct
MNGLSAQHERIAPARHECVYTSCYSEENVWKLCEHIKTSKRNPLGDVYAVFISNERKMVPIWKQKSGRGEEPVIWDYHVILLHDCHKEQTFIYDLDTTLPFPCPFDTYVKEAFKSDNYINPAFWRKLRVVPADVFLQNFASDRSRLKDASGGWRMPPPPYPCIETAESRMNLDDFISMNPSVGWGHVYTLEEFVQHFGKT

SEQ ID NO: 850 - protein, 200 aa, synthetic construct
MNGLSAQHERIAPARHECVYTSCYSEENVWKLCEHIKTSKRCPLGDVYAVFISNERKMVPIWKQKSGRGEEPVIWFYHVILLHDCHKEQTFIYDLDTTLPFPCPFDTYVKETFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 851 - protein, 200 aa, synthetic construct
MNGLSAQHERIAPARHECVYTSCYSEENVWKLCEHIKTSKRCPLGDVYAVFISNERKMVPIWKQKSGRGEEPVIWFYHVILLHDCHKEQTFIYDLDTTLPFPCPFDTYVKETFKSDNYINPAFWRKLRVVPADVFLQNFASDRSHMKDASGGWRMPPPPYPCIETAESRMNLDDFISMNPSVGWGHVYTLEQFVQHFGKT

SEQ ID NO: 852 - protein, 200 aa, synthetic construct
MNGLSAQHERIAPARHECVYTSCYSEENVWKLCEHIKTSKRCPLGDVYAVFISNERKMVPIWKQKSGRGEEPVIWFYHVILLHDCHKEQTFIYDLDTTLPFPCPFDTYVKEAFKSDNYINPAFWRKLRVVPADVFLQNFASDRSHMKDASGGWRMPPPPYPCIETGESRMNLDDFISMNPSVGWGHVYTLEEFVQHFGKT

SEQ ID NO: 853 - protein, 200 aa, synthetic construct
MNGLSAQHERIAPARHECVYTSCYSEENVWKLCEHIKTSKRCPLGDVYAVFISNERKMVPIWKQKSGRGEEPVIWFYAVILLHDCHKEQTFIYDLDTTLPFPCPFDTYVKETFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 854 - protein, 200 aa, synthetic construct
MNGLSAQHERIAPARHECVYTSCYSEENVWKLCEHIKTSKRCPLGDVYAVFISNERKMVPIWKQKSGRGEEPVIWDYHVILLHDCHKEQTFIYDLDTTLPFPCPFDTYVKEAFKSDNYINPAFWRKLRVVPADVFLQNFASDRSRMKDASGGWRMPPPPYPCIETGESRMNLDDFISMNPSVGWGHVYTLEEFVQHFGKT

SEQ ID NO: 855 - protein, 200 aa, synthetic construct
MNGLSAQHERIAPARHECVYTSCYSEENVWKLCEHIKTSKRCPLGDVYAVFISNERKMVPIWKQKSGRGEEPVIWDYHVILLHDCHKEQTFIYDLDTTLPFPCPFDTYVKEAFKSDNYINPAFWRKLRVVPADVFLQNFASDRSRLKDASGGWRMPPPPYPCIETAESRMNLDDFISMNPSVGWGHVYTLEEFVQHFGKT

SEQ ID NO: 856 - protein, 200 aa, synthetic construct
MNGLSAQHERIAPARHECVYTSCYSEENVWKLCEHIKTSKRCPLGDVYAVFISNERKMVPIWKQKSGRGEEPVIWDYHVILLHDCHKEQTFIYDLDTTLPFPCPFDTYVKEAFKSDNYINPAFWRKLRVVPADVFLQNFASDRSRLKDASGGWRMPPPPYPCIETGESRMNLDDFISMNPSVGWGHVYTLEEFVQHFGKT

SEQ ID NO: 857 - protein, 200 aa, synthetic construct
MNGLSAQHERIAPARHECVYTSCYSEENVWKLCEHIKTSKRCPLGDVYAVFISNERKMVPIWKQKSGRGEEPVIWDYHVILLHDCHKEQTFIYDLDTTLPFPCPFDTYVKEAFKSDNYINPAFWRKLRVVPADVFLQNFASDRSRLKDASGGWRMPPPPYPCIETAESRMNLDDFISMNPSVGWGHVYTLEQFVQHFGKT

SEQ ID NO: 858 - protein, 200 aa, synthetic construct
MNGLSAQHERIAPARHECVYTSCYSEENVWKLCEHIKTSKRCPLGDVYAVFISNERKMVPIWKQKSGRGEEPVIWDYHVILLHDCHKEQTFIYDLDTTLPFPCPFDTYVKEAFKSDNYINPAFWRKLRVVPADVFLQNFASDRSHLKDASGGWRMPPPPYPCIETGESRMNLDDFISMNPSVGWGHVYTLEEFVQHFGKT

SEQ ID NO: 859 - protein, 200 aa, synthetic construct
MNGLSAQHERIAPARHECVYTSCYSEENVWKLCEHIKTSKRCPLGDVYAVFISNERKMVPIWKQKSGRGEEPVIWFYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 860 - protein, 200 aa, synthetic construct
MNGLSAQHERIAPARHECVYTSCYSEENVWKLCEHIKTSKRCPLGDVYAVFISNERKMVPIWKQRSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 861 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 862 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYILPAFWRKLRVVPADVFLQNFASDRSHMKDASGGWRMPPPPYPCIETAESRMNLDDFISMNPSVGWGHVYTLEEFVQHFGKT

SEQ ID NO: 863 - protein, 200 aa, synthetic construct
MNGLSAQHERIAPARHECVYTSYYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 864 - protein, 200 aa, synthetic construct
MNGLSAQHERIAPARHECVYTSCYSEENVWKLCEHIKTSKRCPLGDFYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 865 - protein, 200 aa, synthetic construct
MNGLSAQHERIAPARHECVYTFCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 866 - protein, 200 aa, synthetic construct
MNGLSAQHERIAPARHECVYTSCYSEENVWKLCEHIKTSKRCPLGDVYAVFISNERKMVPIWKQRSGRGEEPVIWDYQVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 867 - protein, 200 aa, synthetic construct
MNGLSAQHERITPARHECVYTSCYSEENVWKLCEHIKTSKRCPLGDVYAVFISNERKMVPIWKQRSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 868 - protein, 200 aa, synthetic construct
MNGLSAQHERITPARHECVYTFCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 869 - protein, 200 aa, synthetic construct
MNGLSAQHERIAPARHECVYTPCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 870 - protein, 200 aa, synthetic construct
MNGLSAQHERVAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 871 - protein, 200 aa, synthetic construct
MNGLSAQHERILPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 872 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVRADVFLQNFASDRSHMKDASGGWRMPPPPYPCIETAESRMNLDDFISMNPSVGWGHVYTLEEFVQHFGKT

SEQ ID NO: 873 - protein, 200 aa, synthetic construct
MNGLSAQHERIAPARHECVYTSCYSEENVWKLCEHIKTSKRCPLGDVYAVFISNERKMVPIWKQKVGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 874 - protein, 200 aa, synthetic construct
MNGLSAQHERIAPARHECVYTSCYSEENVWKLCEHIKTSKRCPLGDVYAVFISNERKMVPIWKQKSGRGERPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 875 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDTHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 876 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHACHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 877 - protein, 200 aa, synthetic construct
MNGLSAQHERIAPARHECVYTSCYSEENVWKLCEHIKTSKRCPLGDVYAVFISNERKMVPIWKQKSGRGGEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 878 - protein, 200 aa, synthetic construct
MNGLSAQHERIAPARHECVYTSCYSEENVWKLCEHIKTSKRCPLGDVYAVFISNERKMVPIWKQKSGRGNEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 879 - protein, 200 aa, synthetic construct
MNGLSAQHERIAPARHECVYTSCYSEENVWKLCEHIKTQKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 880 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDPHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 881 - protein, 200 aa, synthetic construct
MNGLSAQHERIAPARHECVYTSCYSEENVWKLCEHIKTSKRCPLGDVYAVFISNERKMVPIWKQKSCRGEEPVIWDYQVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 882 - protein, 200 aa, synthetic construct
MNGLSAQHERIAPARHECVYTSCYTEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 883 - protein, 200 aa, synthetic construct
MNGLSAQHERIAPARHECVYTSCYYEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 884 - protein, 200 aa, synthetic construct
MNGLSAQHERIAPARHECVYTSCYSEENVWKLCEHIKTSKRCPLGDVYAVFISNEQKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 885 - protein, 200 aa, synthetic construct
MNGLSAQHERIAPARHECVYTSCYSEENVWKLCEHIKTSKRCPLGDVYAVFISNELKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 886 - protein, 200 aa, synthetic construct
MNGLSAQHERIAPARHECVYTSCYSEENVWKLCEHIKTSKRCPLGDVYAVFISNEIKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 887 - protein, 200 aa, synthetic construct
MNGLSAQHERIAPARHECVYTSCYSEENVWKLCEHIKTSKRCPLGDVYAVFISNERNMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 888 - protein, 200 aa, synthetic construct
MNGLSAQHERIAPARHECVYTSCYSEENVWKLCEHIKTSKRCPLGDVYAVFISNERLMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 889 - protein, 200 aa, synthetic construct
MNGLSAQHERIAPARHECVYTSCYSEENVWKLCEHIKTSKRCPLGDVYAVFISNEREMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 890 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWQMPPPPYPCIETAESRMNLDDFISMNPSVGWGHVYTLEEFVQHFGKT

SEQ ID NO: 891 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWLMPPPPYPCIETAESRMNLDDFISMNPSVGWGHVYTLEEFVQHFGKT

SEQ ID NO: 892 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWCMPPPPYPCIETAESRMNLDDFISMNPSVGWGHVYTLEEFVQHFGKT

SEQ ID NO: 893 - protein, 200 aa, synthetic construct
MNGLSAQHERIAPARHECVYTSCYCEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 894 - protein, 200 aa, synthetic construct
MNGLSAQHERILPARHECVYTSCYSEENVWKLCEHIKTSKRCPLGDVYAVFISNERKMVPIWKQRSGRGEEPVIWDYQVILLHDCHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 895 - protein, 200 aa, synthetic construct
MNGLSAQHERITPARHECVYTSFYSEENVWKLCEHIKTSKRCPLGDVYAVFISNERKMVPIWKQKVGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 896 - protein, 200 aa, synthetic construct
MNGLSAQHERIALARHECVYTSCYSEENVWKLCEHIKTSKLCPLGDVYAVFISNERKMVPIWKQKSCRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 897 - protein, 200 aa, synthetic construct
MNGLSAQHERILPARHECVYTSCYSEENVWKLCEHIKTSKRCPLGDVYAVFISNERKMVPIWKQRSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 898 - protein, 200 aa, synthetic construct
MNGLSAQHERIAPARHECVYTSYYSEENVWKLCEHIKTQKRCPLGDVYAVFISNERKMVPIWKQKSCRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 899 - protein, 200 aa, synthetic construct
MNGLSAQHERIALARHECIYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYILPAFWRKLRVVPADVFLQNFASDRSHMKDASGGWRMPPPPYPCIETAESRMNLDDFISMNPSVGWGHVYTLEEFVQHFGKT

SEQ ID NO: 900 - protein, 200 aa, synthetic construct
MNGLSAQHERIAPARHECVYTSCYSEENVWKLCEHIKTQKRCPLGDVYAVFISNERKMVPIWKQKSGRGEEPVIWDYQVILLHDTHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 901 - protein, 200 aa, synthetic construct
MNGLSAQHERIAPARHECVYTSFYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 902 - protein, 200 aa, synthetic construct
MNGLSAQHERIAPARHECVYTSCYSEENVWKLCEHIKTSKLCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 903 - protein, 200 aa, synthetic construct
MNGLSAQHERIAPARHECVYTSCYSEENVWKLCEHIKTSKRCPLGDVYAVFISNERKMVPIWKQKSGRGEEPVIWDYRVILLHDCHKEQTFIYDLNTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 904 - protein, 200 aa, synthetic construct
MNGLSAQHERIAPARHECVYTSCYSEENVWKLCEHIKTSKRCPLGDVYAVFISNERKMVPIWKQKSGRGEEPVIWDYRVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 905 - protein, 200 aa, synthetic construct
MNGLSAQHERIAPARHECVYTPCYGEENIWHLCQHIKTSKRCPLGDVYAVFISNERKMVPIWKQKSGRGEEPIIWDYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 906 - protein, 200 aa, synthetic construct
MNGLSAQHERIAPARHECVYTSCYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDSSGGWRMPPPPYPCIETAESRMNLDDFISMNPSVGWGHVYTLEEFVQHFGKT

SEQ ID NO: 907 - protein, 200 aa, synthetic construct
MNGLSAQHERIAPARHECVYTSCYSEENVWKLCEHIKTSKRCPLGDVYAVFISNERKMVPIWKQKSGRGEEPVFWDYQVILLHDCHKEQTFIYDLNTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 908 - protein, 200 aa, synthetic construct
MNGLSAQHERIAPARHECVYTSQYSEENVWKLCEHIKTSKRCPLGDVYAVFISNERKMVPIWKQKSGRGEEPVIWDYQVILLHDCHKEQTFIYDLNTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 909 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLNTTLPFPCPFDTYVKEAFKSDNYINPAFWRKLRVVPADVFLQNFASDRSHMKDAWGGWRMPPPPYPCIETAESRMNLDDFISMNPSVGWGHVYTLEEFVQHFGKT

SEQ ID NO: 910 - protein, 200 aa, synthetic construct
MNGLSAQHERIAPARHECVYTSCYSEVNVWKLCEHIKTSKRCPLGDVYAVFISNERKMVPIWKQKSGRGEEPVIWDYQVIALHDCHKEQTFIYDLNTTLPFPCPFDTYVKETFKSDNYINPAFWRKLRVVPADVFLQNFASDRSHMKDDSGGWRMPPPPYPCIETAESRMNLDDFISMNPSVGWGHVYTLEEFVQHFGKT

SEQ ID NO: 911 - protein, 200 aa, synthetic construct
MNGLSAQHERIAPARHECVYTSCYSEGNVWKLCEHIKTSKRNPLGNVYAVFISNERKMVPIWKQKSGRGEEPVIWDYWVILLHDCHKEQTFIYDLDTTLPFPCPFDTYVKETFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 912 - protein, 200 aa, synthetic construct
MNGLSAQHERIAPARHECVYTSCYSWENVWKLCEHIKTSKRNPLGDVYAVFISNERKMVPIWKQKSGRGEEPVIWDYQVGLLHDCHKEQTFIYDLDTTLPFPCPFDTYVKETFKSDNYINPAFWRKLRVVPADVFLQNFASDRSHMKLASGGWRMPPPPYPCIETAESRMNLDDFISMNPSVGWGHVYTLEEFVQHFGKT

SEQ ID NO: 913 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLNTTLPFPCPFDTYVKEAFKSDNYINPAFWRKLRVVPADVFLQNFASDRSHMKDARGGWRMPPPPYPCIETAESRMNLDDFISMNPSVGWGHVYTLEEFVQHFGKT

SEQ ID NO: 914 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQAILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 915 - protein, 200 aa, synthetic construct
MNGLSAQHERIAPARHECVYTSCTSKENVWKLCEHIKTSKRCPLGDVYAVFISNERKMVPIWKQKSGRGEEPVIWFYQVILLHDCHKEQTFIYDLNTTLPFPCPFDTYVKEAFKSDNYINPAFWRKLRVVPADVFLQNFASDRSHMKDAAGGWRMPPPPYPCIETAESRMNLDDFISMNPSVGWGHVYTLEEFVQHFGKT

SEQ ID NO: 916 - protein, 204 aa, synthetic construct
MEGNGPAAVHYQPASPPRDACVYTACYGEENIWHLCQYIKNHDQYPLEECYAVFISNERKMIPIWKQQARPGDGPIIWDYHVLLLHVSSGGQSFIYDLDTVLPFPCLFDTYVEDAIKSDDDIHPQFRRKFRVICADSYLKNFASDRSHMKDSSGNWREPPPPYPCIETGDSKMNLNDFISMDPKVGWGAVYTLSEFTHRFGSKN

SEQ ID NO: 917 - protein, 204 aa, synthetic construct
MEGNGPAAVHYQPASPPRDACVYTACYAEENIWHLCQYIKNHDQYPLEECYAVFISNERKMIPIWKQQARPGDGPIIWDYHVVLLHVSSGGQSFIYDLDTVLLFPCLFDTYVEDAIKSDDDIHPQFRRKFRVICADSYLKNFASDRSHMKDSSGNWREPPPPYPCIETGDSKMNLNDFISMDPKVGWGAVYTLSEFTHRFGSKN

SEQ ID NO: 918 - protein, 204 aa, synthetic construct
MEGNGPAAVHYQPASPPRDACVYSSCYSEENVWKLCEYIKNHDQYPLEECYAVFISNERKMIPIWKQQARPGDGPVIWDYRVVLLHVSSGGQSFIYDLDTVLPFPCLFDTYVEDAIKSDDDILPQFRRKFRVICADSYLKNFASDRSHMKDSSGNWREPPPPYPCIETGDSKMNLNDFISMDPKVGWGAVYTLSEFTHRFGSKN

SEQ ID NO: 919 - protein, 204 aa, synthetic construct
MEGNGPAAVHYQPASPPRDAGVYSSCYSEENVWKLCEYIKNHDQYPLEKCYAVFISNERKMIPIWKQQARPGDGPVIRDYQVVLLHVSSGGQSFIYDLDTVLPFPCLFDTYVEDAIKSDDDIHPQFRRKFRVICADSYLKNFASDRSHMKDSSGNWREPPPPYPCIETGDSKMNLNDFISMDPKVGWGAVYTLSEFTHRFGSKN

SEQ ID NO: 920 - protein, 204 aa, synthetic construct
MEGNGPAAVHYQPASPPRDACVYSSCYSEENVWKLCEYIKNHDQYPLEECYAVFISSERKMIPIWKQQARPGDGPVIWDYRVVLLHVSSGGQSLIYDLDTVLPFPCLFDTYVEDAIKSDDDIHPQFRRKFRVICADSYLKNFASDRSHMKDSSGNWREPPPPYPCIETGDSKMNLNDFISMDPKVGWGAVYTLSEFTHRFGSKN

SEQ ID NO: 921 - protein, 204 aa, synthetic construct
MEGNGPAAVHYQPASPPRDACVYSSCYSEENVWKLCEYIKNHDQYPLEECYAVFISNERKMIPIWKQQARPGDGPVIWDYRVVLLHVSSGGQSFIYDLDTVLPFPCLFDTYVEDTIESDDDIHPQFRRKFRVICADSYLKNFASDRSHMKDSSGNWREPPPPYPCIETGDSKMNLNDFISMDPKVGWGAVYTLSEFTHRFGSKN

SEQ ID NO: 922 - protein, 204 aa, synthetic construct
MEGNGPAAVHYQPASPPRDACVYSSCYSEENVWKLCEYIKNHDQYPLEGCYAVFISNERKMIPIWKQQARPGDGPVIWDYQVVLLHVSSGGQSFIYDLDTVLPFPCLFDTYVEDTIKSDDDIHSQFRRKFRVICADSYLKNFASDRSHMKDSSGNWREPPPPYPCIETGDSKMNLNDFISMDPKVGWGAVYTLSEFTHRFGSKN

SEQ ID NO: 923 - protein, 204 aa, synthetic construct
MEGNGPAAVHYQPASPPRDACVYSPCYGEENVWKLCEYIKNHDQYPLEECYAVFISNERKMIPIWKQQARPGDGPIIWDYHVVLLHVSSGGQSFIYDLDTVLPFPCLFDTYVEDAIKSDDDIHPQFRRKFRVICADSYLKNFASDRSHMKDSSGNWREPPPPYPCIETGDSKMNLNDFISMDPKVGWGAVYTLSEFTHRFGSKN

SEQ ID NO: 924 - protein, 204 aa, synthetic construct
MEGNGPAAVHYQPASPPRDACVYSSCYREENVWKLCEYIKNHDQYPLEECYAVFISNERKIIPIWKQQARPGDGPVIWDYQVVLLHVSSGGQSFIYDLDTVLPFPCLFDTYVEDAIMSDDDIHPQFRRKFRVICADSYLKNFASDRSHMKDSSGNWREPPPPYPCIETGDSKMNLNDFISMDPKVGWGAVYTLSEFTHRFGSKN

SEQ ID NO: 925 - protein, 204 aa, synthetic construct
MEGNGPAAVHYQPASPPRDACVYSSCYSEENVWKLCEYIKNHDLYPLEECYAVFISNERKMIPIWKQQARPGDGPVIWDYQVVLLHVSSGGQSFIYDLDTVLPFPCLFDTYVEDAIKSDDDIHPQFRRKFRVICADSYLKNFASDRSHMKDSSGNWLEPPPPYPCIETGDSKMNLNDFISMDPKVGWGAVYTLSEFTHRFGSKN

SEQ ID NO: 926 - protein, 204 aa, synthetic construct
MEGNGPAAVHYQPASPPRDACVYSSCYSEENVWKLCEYIKNHDQYPLEECYAVFISNERKMIPIWKQQARPGDGPVIWDYRVVLLHVSSGGQSFIYDLDTVLPFPCLFDTYVEDAIKSDDDIHPQFRRKFRVICADSYLKNFASDRSHMKDSSGNWREPPPPYPCIETGDSKMNLNDFISMDPKVGWGAVYTLSEFTHRFGSKN

SEQ ID NO: 927 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLNTTLPFPCPFDTYVKEAFKSDNYINPAFWRKLRVVPADVFLQNFASDRSHMKDGSGGWRMPPPPYPCIETAESRMNLDDFISMNPSVGWGHVYTLEEFVQHFGKT

SEQ ID NO: 928 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLNTTLPFPCPFDTYVKEAFKSDNYINPAFWRKLRVVPADVFLQNFASDRSHMKDVSGGWRMPPPPYPCIETAESRMNLDDFISMNPSVGWGHVYTLEEFVQHFGKT

SEQ ID NO: 929 - protein, 200 aa, synthetic construct
MNGLSAQHERIAPARHECVYTSCYSEENVWKLCEHIKTSKRCPLGDVYAVFISNERKMVPIWKQKSGRGEEPVFWDYQVILLHDCHKEQTFIYDLNTTLPFPCPFDTYVKEAFKSDNYINPAFWRKLRVVPADVFLQNFASDRSHMRDASGGWRMPPPPYPCIETAESRMNLDDFISMNPSVGWGHVYTLEEFVQHFGKT

SEQ ID NO: 930 - protein, 200 aa, synthetic construct
MNGLSAQHERIAPARHECVYTSCYSEENVWKLCEHIKTSKRCPLGDVYAVFISNERKMVPIWKQKSGRGEEAVIWDYQVILLHDCHKEQTFIYDLNTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 931 - protein, 200 aa, synthetic construct
MNGLSAQHERIAPARHECVYTSGYSEENVWKLCEHIKTSKRCPLGDVYAVFISNERKMVPIWKQKSGRGEEPVFWDYQVILLHDCHKEQTFIYDLNTTLPFPCPFDTYVKEAFKSDNYINPAFWRKLRVVPADVFLQNFASDRSHMRDASGGWRMPPPPYPCIETAESRMNLDDFISMNPSVGWGHVYTLEEFVQHFGKT

SEQ ID NO: 932 - protein, 200 aa, synthetic construct
MNGLSAQHERIAPARHECVYTSGYSEENVWKLCEHIKTSKRCPLGDVYAVFISNERKMVPIWKQKSGRGEEPVWWDYQVILLHDCHKEQTFIYDLNTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 933 - protein, 200 aa, synthetic construct
MNGLSAQHERIAPARHECVYTSCYSEENVWKLCEHIKTSKRNPLGDVYAVFISNERKMVPIWKQKSGRGEEPEIWDYQVILLHDCHKEQTFIYDLNTTLPFPCPFDTYVKETFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 934 - protein, 200 aa, synthetic construct
MNGLSAQHERIAPARHECVYTSCYSEENVWKLCEHIKTSKRCPLGDVYAVFISNERKMVPIWKQKSGRGEEPVFWDYQVILLHDCHKEQTFIYDLNTTLPFPCPFDTYVKEAFKSDNYINPAFWRKLRVVPADVFLQNFASDRSHMKDDSGGWRMPPPPYPCIETAESRMNLDDFISMNPSVGWGHVYTLEEFVQHFGKT

SEQ ID NO: 935 - protein, 200 aa, synthetic construct
MNGLSAQHERIAPARHECVYTSCYSEENVWKLCEHIKTSKRCPLGDVYAVFISNERKMVPIWKQKSGRGEEPVWWDYQVILLHDCHKEQTFIYDLDTTLPFPCPFDTYVKEAFKSDNYINPAFWRKLRVVPADVFLQNFASDRSHMKNASGGWRMPPPPYPCIETAESRMNLDDFISMNPSVGWGHVYTLEEFVQHFGKT

SEQ ID NO: 936 - protein, 200 aa, synthetic construct
MNGLSAQHERIAPARHECVYTSCYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 937 - protein, 200 aa, synthetic construct
MNGLSAQHERIAPARHECVYTSCYSEENVWKLCEHIKTSKRCPLGDVYAVFISNERKMVPIWKQKSGRGEEPVWWDYQVILLHDCHKEQTFIYDLNTTLPFPCPFDTYVKEAFKSDNYINPAFWRKLRVVPADVFLQNFASDRSHMKDESGGWRMPPPPYPCIETAESRMNLDDFISMNPSVGWGHVYTLEEFVQHFGKT

SEQ ID NO: 938 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFRSDNYINPAFWRKLRVVPADVFLQNFASDRSHMKDASGGWCMPPPPYPCIETAESRMNLDDFISMNPSVGWGHVYTLEEFVQHFGKT

SEQ ID NO: 939 - protein, 200 aa, synthetic construct
MNGLSAQHERIAPARHECVYTFYYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 940 - protein, 200 aa, synthetic construct
MNGLSAQHERIAPARHECVYTFYYSEENVWKLCEHIKTSKRCPLGDVYAVFISNERKMVPIWKQKSGRGERPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 941 - protein, 200 aa, synthetic construct
MNGLSAQHERIAPARHECVYTSYYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYIPPAFWRKLRVVPADVFLQNFASDRSHMKDASGGWRMPPPPYPCIETAESRMNLDDFISMNPSVGWGHVYTLEEFVQHFGKT

SEQ ID NO: 942 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYIPPRFWRKLRVVPADVFLQNFASDRSHMKDPSGGWRMPPPPYPCIETAESRMNLDDFISMNPSVGWGHVYTLEEFVQHFGKT

SEQ ID NO: 943 - protein, 200 aa, synthetic construct
MNGLSAQHERIAPARHECVYTSCYSEENVWKLCEHIKTSKRCPLGDVYAVFISNERKMVPIWKQKSCRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 944 - protein, 200 aa, synthetic construct
MNGLSAQHERIAPARHECVYTTCYSEENVYHLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 945 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDVSGGWRMPPPPYPCIETAESRMNLDDFISMNPSVGWGHVYTLEEFVQHFGKT

SEQ ID NO: 946 - protein, 200 aa, synthetic construct
MNGLSAQHERIAPARHECVYTSCYSEENVWKLCEHIKTSKRCPLGDVYAVFISNERKMVPIWKQKSGRGEEPVFWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 947 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVVLLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 948 - protein, 200 aa, synthetic construct
MNGLSAQHERIAPARHECVYTSCYSEENVWKLCEHIKTSKRCPLGDVYAVFISNERKMVPIWKQKSGRGEECVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 949 - protein, 200 aa, synthetic construct
MNGLSAQHERIAPARHECVYTSCYGEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 950 - protein, 200 aa, synthetic construct
MNGLSAQHERIAPARHECVYTSCYSEENVWKLCEHIKTSKRCPLGDVYAVFISNERKMVPIWKQKSGRGEEPCIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 951 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDCSGGWRMPPPPYPCIETAESRMNLDDFISMNPSVGWGHVYTLEEFVQHFGKT

SEQ ID NO: 952 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDRSGGWRMPPPPYPCIETAESRMNLDDFISMNPSVGWGHVYTLEEFVQHFGKT

SEQ ID NO: 953 - protein, 200 aa, synthetic construct
MNGLSAQHERIAPARHECVYTECYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 954 - protein, 200 aa, synthetic construct
MNGLSAQHERIAPARHECVYTSCYSEENVWKLCEHIKTSKRCPLGDVYAVFISNERKMVPIWKQKSGRGEEPEIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 955 - protein, 200 aa, synthetic construct
MNGLSAQHERIAPARHECVYTSCYSEENVWKLCEHIKTSKRCPLGDVYAVFISNERKMVPIWKQKSGRGEEPVIYDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 956 - protein, 200 aa, synthetic construct
MNGLSAQHERIAPARHECVYTSCYSEENVWKLCEHIKTSKRCPLGDVYAVFISNERKMVPIWKQKSGRGEEPVLWDYQVILLHDCHKEQTFIYDLDTTLPFPCPFDTYVKEAFKSDNYINPAFWRKLRVVPADVFLQNFASDRSHMKDGSGGWRMPPPPYPCIETAESRMNLDDFISMNPSVGWGHVYTLEEFVQHFGKT

SEQ ID NO: 957 - protein, 200 aa, synthetic construct
MNGLSAQHERIAPARHECVYTSCYSEENVWKLCEHIKTSKRCPLGDVYAVFISNERKMVPIWKQKSGRGEEPVVWDYQVILLHDCHKEQTFIYDLDTTLPFPCPFDTYVKEAFKSDNYINPAFWRKLRVVPADVFLQNFASDRSHMKDESGGWRMPPPPYPCIETAESRMNLDDFISMNPSVGWGHVYTLEEFVQHFGKT

SEQ ID NO: 958 - protein, 200 aa, synthetic construct
MNGLSAQHERIAPARHECVYTSCYSEENVWKLCEHIKTSKRCPLGDVYAVFISNERKMVPIWKQKSGRGEERVIWDYQVILLHDCHKEQTFIYDLDTTLPFPCPFDTYVKEAFKSDNYINPAFWRKLRVVPADVFLQNFASDRSHMKDQSGGWRMPPPPYPCIETAESRMNLDDFISMNPSVGWGHVYTLEEFVQHFGKT

SEQ ID NO: 959 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRVHMKDASGGWRMPPPPYPCIETAESRMNLDDFISMNPSVGWGHVYTLEEFVQHFGKT

SEQ ID NO: 960 - protein, 200 aa, synthetic construct
MNGLSAQHERIAPARHECVYTSCYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDARGGWRMPPPPYPCIETAESRMNLDDFISMNPSVGWGHVYTLEEFVQHFGKT

SEQ ID NO: 961 - protein, 200 aa, synthetic construct
MNGLSAQHERIAPARHECVYTSCYGEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDARGGWRMPPPPYPCIETAESRMNLDDFISMNPSVGWGHVYTLEEFVQHFGKT

SEQ ID NO: 962 - protein, 200 aa, synthetic construct
MNGLSAQHERIAPARHECVYTSQYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 963 - protein, 200 aa, synthetic construct
MNGLSAQHERIAPARHECVYTSQYGEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 964 - protein, 200 aa, synthetic construct
MNGLSAQHERIAPARHECVYTSCYGEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 965 - protein, 200 aa, synthetic construct
MNGLSAQHERIAPARHECVYTPCYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 966 - protein, 200 aa, synthetic construct
MNGLSAQHERIAPARHECVYTSCYSEENVWKLCEHIKTSKRCPLGDVYAVFISNERKMVPIWKQKSGRGEEPIIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 967 - protein, 200 aa, synthetic construct
MNGLSAQHERIAPARHECVYTSCYGEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDSSGGWRMPPPPYPCIETAESRMNLDDFISMNPSVGWGHVYTLEEFVQHFGKT

SEQ ID NO: 968 - protein, 200 aa, synthetic construct
MNGLSAQHERIAPARHECVYTSCYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDGSGGWRMPPPPYPCIETAESRMNLDDFISMNPSVGWGHVYTLEEFVQHFGKT

SEQ ID NO: 969 - protein, 200 aa, synthetic construct
MNGLSAQHERIAPARHECVYTSCYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDVSGGWRMPPPPYPCIETAESRMNLDDFISMNPSVGWGHVYTLEEFVQHFGKT

SEQ ID NO: 970 - protein, 200 aa, synthetic construct
MNGLSAQHERIAPARHECVYTSCYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDDSGGWRMPPPPYPCIETAESRMNLDDFISMNPSVGWGHVYTLEEFVQHFGKT

SEQ ID NO: 971 - protein, 200 aa, synthetic construct
MNGLSAQHERIAPARHECVYTSCYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDESGGWRMPPPPYPCIETAESRMNLDDFISMNPSVGWGHVYTLEEFVQHFGKT

SEQ ID NO: 972 - protein, 200 aa, synthetic construct
MNGLSAQHERIAPARHECVYTPCYGEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 973 - protein, 200 aa, synthetic construct
MNGLSAQHERIAPARHECVYTSCYGEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDSSGGWRMPPPPYPCIETAESRMNLDDFISMNPSVGWGHVYTLEEFVQHFGKT

SEQ ID NO: 974 - protein, 200 aa, synthetic construct
MNGLSAQHERIAPARHECVYTSCYGEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDARGGWRMPPPPYPCIETAESRMNLDDFISMNPSVGWGHVYTLEEFVQHFGKT

SEQ ID NO: 975 - protein, 200 aa, synthetic construct
MNGLSAQHERIAPARHECVYTSCYSEENVWHLCQHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 976 - protein, 200 aa, synthetic construct
MNGLSAQHERIAPARHECVYTPCYGEENVWKLCEHIKTSKRCPLGDVYAVFISNERKMVPIWKQKSGRGEEPIIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 977 - protein, 200 aa, synthetic construct
MNGLSAQHERIAPARHECVYTSCYGEENVWHLCQHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 978 - protein, 200 aa, synthetic construct
MNGLSAQHERIAPARHECVYTPCYGEENIWKLCEHIKTSKRCPLGDVYAVFISNERKMVPIWKQKSGRGEEPIIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 979 - protein, 200 aa, synthetic construct
MNGLSAQHERIAPARHECVYTPCYGEENVWHLCQHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 980 - protein, 200 aa, synthetic construct
MNGLSAQHERIAPARHECVYTSCYGEENIWHLCQHIKTSKRCPLGDVYAVFISNERKMVPIWKQKSGRGEEPIIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 981 - protein, 200 aa, synthetic construct
MNGLSAQHERIAPARHECVYTPCYGEENIWHLCQHIKTSKRCPLGDVYAVFISNERKMVPIWKQKSGRGEEPIIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 982 - protein, 200 aa, synthetic construct
MNGLSAQHERVAPARHECVYTPCYGEENVWKLAQHIKTSKRCPLGDVYAVFISNERKMVPIWKQKSGRGEEPLVWDYHVLLLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 983 - protein, 200 aa, synthetic construct
MNGLSAQHERIAPARHECVYTACYGEENVWKLAQHIKTSKRCPLGDVYAVFISNERKMVPIWKQKSGRGEEPIVWDYHVLLLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 984 - protein, 200 aa, synthetic construct
MNGLSAQHERIAPARHECVYTACYGEENVWHLCGHIKTSKRCPLGDVYAVFISNERKMVPIWKQKSGRGEEPIIWDYHVLLLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 985 - protein, 200 aa, synthetic construct
MNGLSAQHERIAPARHECVYTACYGEENIWHLAQHIKTSKRCPLGDVYAVFISNERKMVPIWKQKSGRGEEPVIWDYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 986 - protein, 200 aa, synthetic construct
MNGLSAQHERIAPARHECVYTPCYAEENVYKLAEHIKTSKRCPLGDVYAVFISNERKMVPIWKQKSGRGEEPIIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 987 - protein, 200 aa, synthetic construct
MNGLSAQHERIAPARHECVYTPCYGEENIWHLAQHIKTSKRCPLGDVYAVFISNERKMVPIWKQKSGRGEEPVIWDYHVLLLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 988 - protein, 200 aa, synthetic construct
MNGLSAQHERIAPARHECVYTPCYGEENIWHLAQHIKTSKRCPLGDVYAVFISNERKMVPIWKQKSGRGEEPILWDYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 989 - protein, 200 aa, synthetic construct
MNGLSAQHERIAPARHECVYTPCYGEENIWHLCEHIKTSKRCPLGDVYAVFISNERKMVPIWKQKSGRGEEPILWDYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 990 - protein, 200 aa, synthetic construct
MNGLSAQHERIAPARHECVYTPCYGEENIWHLCQHIKTSKRCPLGDVYAVFISNERKMVPIWKQKSGRGEEPILWDYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 991 - protein, 200 aa, synthetic construct
MNGLSAQHERIAPARHECVYTPCYGEENVYHLCEHIKTSKRCPLGDVYAVFISNERKMVPIWKQKSGRGEEPIVWDYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 992 - protein, 200 aa, synthetic construct
MNGLSAQHERIAPARHECVYTACYGEENVWKLCQHIKTSKRCPLGDVYAVFISNERKMVPIWKQKSGRGEEPILWDYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 993 - protein, 200 aa, synthetic construct
MNGLSAQHERIAPARHECVYTSCYSEENVWKLCEHIKTSKRCPLGDVYAVFISSERKMVPIWKQKSGRGEEPVIWDYRVILLHDCHKEQTLIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 994 - protein, 200 aa, synthetic construct
MNGLSAQHERIAPARHECVYTSCYSEENVWKLCEHIKTSKRCPLGDVYAVFISNERKMVPIWKQKSGRGEEPVIWDYRVILLHDCHKEQTFIYDLDTTLPFPCPFDTYVKETFESDNYINPAFWRKLRVVPADVFLQNFASDRSHMKDASGGWRMPPPPYPCIETAESRMNLDDFISMNPSVGWGHVYTLEEFVQHFGKT

SEQ ID NO: 995 - protein, 200 aa, synthetic construct
MNGLSAQHERIAPARHECVYTSCYSEENVWKLCEHIKTSKRCPLGGVYAVFISNERKMVPIWKQKSGRGEEPVIWDYQVILLHDCHKEQTFIYDLDTTLPFPCPFDTYVKETFKSDNYINSAFWRKLRVVPADVFLQNFASDRSHMKDASGGWRMPPPPYPCIETAESRMNLDDFISMNPSVGWGHVYTLEEFVQHFGKT

SEQ ID NO: 996 - protein, 200 aa, synthetic construct
MNGLSAQHERIAPARHECVYTPCYGEENVWKLCEHIKTSKRCPLGDVYAVFISNERKMVPIWKQKSGRGEEPIIWDYHVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 997 - protein, 200 aa, synthetic construct
MNGLSAQHERIAPARHECVYTSCYREENVWKLCEHIKTSKRCPLGDVYAVFISNERKIVPIWKQKSGRGEEPVIWDYQVILLHDCHKEQTFIYDLDTTLPFPCPFDTYVKEAFMSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 998 - protein, 200 aa, synthetic construct
MNGLSAQHERIAPARHECVYTSCYSEENVWKLCEHIKTSKLCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWLMPPPPYPCIETAESRMNLDDFISMNPSVGWGHVYTLEEFVQHFGKT

SEQ ID NO: 999 - protein, 200 aa, synthetic construct
MNGLSAQHERILPARHECVYTECYSEENVWKLCEYIKTSKRCPLGDVYAVFISNERKMVPIWKQRSGRGERVVIWDYQVIMLHDCHKEQTFIYDLDTTLPFPCPFDTYVKEAFKSDNYINPRFWRKLRVVRADVFLQNFASDRSHMKDASGGWRMPPPPYPCIETAESRMNLDDFISMNPSVGWGHVYTLEEFVQHFGKT

SEQ ID NO: 1000 - protein, 200 aa, synthetic construct
MNGLSAQHERILPARHECVYTPCYSEENVWKLCEHIKTQKRCPLGDVYAVFISNERKMVPIWKQKVGRGEEPVWWDYQVILLHDCHKEQTFIYDLDTTLPFPCPFDTYVKEAFKSDNYIRPRFWRKLRVVPADVFLQNFASDRSHMKDASGGWRMPPPPYPCIETAESRMNLDDFISMNPSVGWGHVYTLEEFVQHFGKT

SEQ ID NO: 1001 - protein, 200 aa, synthetic construct
MNGLSAQHERILPARHECVYTECYSEENVWKLCEHIKTSKRCPLGDVYAVFISNERKMVPIWKQRSGRGERVVIWDYQVILLHDCHKEQTFIYDLDTTLPFPCPFDTYVKEAFKSDNYIRPRFWRKLRVVPADVFLQNFASDRSHMKDASGGWRMPPPPYPCIETAESRMNLDDFISMNPSVGWGHVYTLEEFVQHFGKT

SEQ ID NO: 1002 - protein, 200 aa, synthetic construct
MNGLSAQHERILPARHECVYTPCYSEENVWKLCEHIKTQKRCPLGDVYAVFISNERKMVPIWKQRSGRGERPVIWDYQVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 1003 - protein, 200 aa, synthetic construct
MNGLSAQHERILPARHECVYTECYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYIRPRFWRKLRVVPADVFLQNFASDRSHMKDASGGWRMPPPPYPCIETAESRMNLDDFISMNPSVGWGHVYTLEEFVQHFGKT

SEQ ID NO: 1004 - protein, 200 aa, synthetic construct
MNGLSAQHERIAPARHECVYTECYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 1005 - protein, 200 aa, synthetic construct
MNGLSAQHERIAPARHECVYTSCYSEENVWKLCEHIKTSKRCPLGDVYAVFISNERKMVPIWKQKSGRGEERVIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 1006 - protein, 200 aa, synthetic construct
MNGLSAQHERIAPARHECVYTSCYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDQSGGWRMPPPPYPCIETAESRMNLDDFISMNPSVGWGHVYTLEEFVQHFGKT

SEQ ID NO: 1007 - protein, 200 aa, synthetic construct
MNGLSAQHERIAPARHECVYTECYSEENVWKLCEHIKTSKRCPLGDVYAVFISNERKMVPIWKQKSGRGEERVIWDYKVILLHDTHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 1008 - protein, 200 aa, synthetic construct
MNGLSAQHERIAPARHECVYTSCYSEENVWKLCEHIKTQKRCPLGDVYAVFISNERKMVPIWKQKSGRGEEPVIWDYKVILLHDTHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 1009 - protein, 200 aa, synthetic construct
MNGLSAQHERIAPARHECVYTECYSEENVWKLCEHIKTQKRCPLGDVYAVFISNERKMVPIWKQKSGRGEEPVIWDYKVILLHDTHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 1010 - protein, 200 aa, synthetic construct
MNGLSAQHERIAPARHECVYTECYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDQSGGWRMPPPPYPCIETAESRMNLDDFISMNPSVGWGHVYTLEEFVQHFGKT

SEQ ID NO: 1011 - protein, 200 aa, synthetic construct
MNGLSAQHERIAPARHECVYTECYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 1012 - protein, 200 aa, synthetic construct
MNGLSAQHERIAPARHECVYTECYSEENVWKLCEHIKTSKRCPLGDVYAVFISNERKMVPIWKQKSGRGEEPVIWDYKVILLHDTHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 1013 - protein, 200 aa, synthetic construct
MNGLSAQHERIAPARHECVYTECYSEENVWKLCEHIKTQKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 1014 - protein, 200 aa, synthetic construct
MNGLSAQHERIAPARHECVYTSCYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDQSGGWRMPPPPYPCIETAESRMNLDDFISMNPSVGWGHVYTLEEFVQHFGKT

SEQ ID NO: 1015 - protein, 200 aa, synthetic construct
MNGLSAQHERIAPARHECVYTSCYSEENVWKLCEHIKTSKRCPLGDVYAVFISNERKMVPIWKQKSGRGEEPVIWDYKVILLHDTHKEQTFIYDLDTTLPFPCPFDTYVKEAFKSDNYINPAFWRKLRVVPADVFLQNFASDRSHMKDQSGGWRMPPPPYPCIETAESRMNLDDFISMNPSVGWGHVYTLEEFVQHFGKT

SEQ ID NO: 1016 - protein, 200 aa, synthetic construct
MNGLSAQHERIAPARHECVYTSCYSEENVWKLCEHIKTQKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDQSGGWRMPPPPYPCIETAESRMNLDDFISMNPSVGWGHVYTLEEFVQHFGKT

SEQ ID NO: 1017 - protein, 200 aa, synthetic construct
MNGLSAQHERIAPARHECVYTECYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDQSGGWRMPPPPYPCIETAESRMNLDDFISMNPSVGWGHVYTLEEFVQHFGKT

SEQ ID NO: 1018 - protein, 200 aa, synthetic construct
MNGLSAQHERIAPARHECVYTECYSEENVWKLCEHIKTSKRCPLGDVYAVFISNERKMVPIWKQKSGRGEEPVIWDYKVILLHDTHKEQTFIYDLDTTLPFPCPFDTYVKEAFKSDNYINPAFWRKLRVVPADVFLQNFASDRSHMKDQSGGWRMPPPPYPCIETAESRMNLDDFISMNPSVGWGHVYTLEEFVQHFGKT

SEQ ID NO: 1019 - protein, 200 aa, synthetic construct
MNGLSAQHERIAPARHECVYTECYSEENVWKLCEHIKTQKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDQSGGWRMPPPPYPCIETAESRMNLDDFISMNPSVGWGHVYTLEEFVQHFGKT

SEQ ID NO: 1020 - protein, 200 aa, synthetic construct
MNGLSAQHERIAPARHECVYTECYSEENVWKLCEHIKTSKRCPLGDVYAVFISNERKMVPIWKQKSGRGEEPVIWDYKVILLHDTHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 1021 - protein, 200 aa, synthetic construct
MNGLSAQHERIAPARHECVYTECYSEENVWKLCEHIKTQKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 1022 - protein, 200 aa, synthetic construct
MNGLSAQHERIAPARHECVYTECYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDVSGGWRMPPPPYPCIETAESRMNLDDFISMNPSVGWGHVYTLEEFVQHFGKT

SEQ ID NO: 1023 - protein, 200 aa, synthetic construct
MNGLSAQHERIAPARHECVYTECYSEENVWKLCEHIKTQKRCPLGDVYAVFISNERKMVPIWKQKSGRGEEPVIWDYKVILLHDTHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 1024 - protein, 200 aa, synthetic construct
MNGLSAQHERIAPARHECVYTECYSEENVWKLCEHIKTSKRCPLGDVYAVFISNERKMVPIWKQKSGRGEEPVIWDYKVILLHDTHKEQTFIYDLDTTLPFPCPFDTYVKEAFKSDNYINPAFWRKLRVVPADVFLQNFASDRSHMKDVSGGWRMPPPPYPCIETAESRMNLDDFISMNPSVGWGHVYTLEEFVQHFGKT

SEQ ID NO: 1025 - protein, 200 aa, synthetic construct
MNGLSAQHERIAPARHECVYTECYSEENVWKLCEHIKTQKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDVSGGWRMPPPPYPCIETAESRMNLDDFISMNPSVGWGHVYTLEEFVQHFGKT

SEQ ID NO: 1026 - protein, 27 aa, synthetic construct
GGGSGGGSGGGSGLNDFFEAQKIEWHE

SEQ ID NO: 1027 - protein, 54 aa, synthetic construct
GGGSGGGSGGGSGLNDFFEAQKIEWHEGGGSGGGSGGGSGLNDFFEAQKIEWHE

SEQ ID NO: 1028 - protein, 56 aa, synthetic construct
GSGGGSGGGSGGGSGLNDFFEAQKIEWHEGGGSGGGSGGGSGLNDFFEAQKIEWHE

SEQ ID NO: 1029 - protein, 38 aa, synthetic construct
GHHHHHHHHHHGGGSGGGSGGGSGLNDFFEAQKIEWHE

SEQ ID NO: 1030 - protein, 65 aa, synthetic construct
GHHHHHHHHHHGGGSGGGSGGGSGLNDFFEAQKIEWHEGGGSGGGSGGGSGLNDFFEAQKIEWHE

SEQ ID NO: 1031 - protein, 67 aa, synthetic construct
GGSHHHHHHHHHHGGGSGGGSGGGSGLNDFFEAQKIEWHEGGGSGGGSGGGSGLNDFFEAQKIEWHE

SEQ ID NO: 1032 - protein, 66 aa, synthetic construct
GSHHHHHHHHHHGGGSGGGSGGGSGLNDFFEAQKIEWHEGGGSGGGSGGGSGLNDFFEAQKIEWHE

SEQ ID NO: 1033 - protein, 61 aa, synthetic construct
GGGSGGGSGGGSGLNDFFEAQKIEWHEGGGSGGGSGGGSGLNDFFEAQKIEWHEGHHHHHH

SEQ ID NO: 1034 - protein, 14 aa, synthetic construct
FAAWAAYAAAADDD

SEQ ID NO: 1035 - protein, 15 aa, synthetic construct
LAQFASIAAYASDDD

SEQ ID NO: 1036 - protein, 9 aa, synthetic construct
DQQRLIFAG

SEQ ID NO: 1037 - protein, 22 aa, synthetic construct
DQQIASSRLAASFAAQQYPDDD

SEQ ID NO: 1038 - protein, 13 aa, synthetic construct
RLAFSALGAADDD

SEQ ID NO: 1039 - protein, 7 aa, synthetic construct
EFIAWLV

SEQ ID NO: 1040 - protein, 10 aa, synthetic construct
RLAFAYPDDD

SEQ ID NO: 1041 - protein, 10 aa, synthetic construct
RLIFAYPDDD

SEQ ID NO: 1042 - protein, 10 aa, synthetic construct
RLVFAYPDDD

SEQ ID NO: 1043 - protein, 10 aa, synthetic construct
RLMFAYPDDD

SEQ ID NO: 1044 - protein, 10 aa, synthetic construct
  MOD_RES 3: note = methionine sulfoxide
RLMFAYPDDD

SEQ ID NO: 1045 - protein, 10 aa, synthetic construct
DQQRLIFAGK

SEQ ID NO: 1046 - protein, 8 aa, synthetic construct
EFIAWLVK

SEQ ID NO: 1047 - protein, 4 aa, synthetic construct
MAKL

SEQ ID NO: 1048 - protein, 4 aa, synthetic construct
AAKL

SEQ ID NO: 1049 - protein, 4 aa, synthetic construct
RAKL

SEQ ID NO: 1050 - protein, 4 aa, synthetic construct
YREL

SEQ ID NO: 1051 - protein, 4 aa, synthetic construct
  MOD_RES 2: note = asymmetric dimethylarginine
YREL

SEQ ID NO: 1052 - protein, 8 aa, synthetic construct
  MOD_RES 2: note = symmetric dimethylarginine
YRSDMAEL

SEQ ID NO: 1053 - protein, 12 aa, synthetic construct
LRLAFAYPDDDK

SEQ ID NO: 1054 - protein, 12 aa, synthetic construct
  MOD_RES 2: note = may be deiminated to form citrulline
LRLAFAYPDDDK

SEQ ID NO: 1055 - protein, 5 aa, synthetic construct
LRLAF

SEQ ID NO: 1056 - protein, 5 aa, synthetic construct
  MOD_RES 2: note = may be deiminated to form citrulline
LRLAF

SEQ ID NO: 1057 - protein, 11 aa, synthetic construct
RLTFIAYPDDD

SEQ ID NO: 1058 - protein, 11 aa, synthetic construct
  MOD_RES 3: note = phosphothreonine
RLTFIAYPDDD

SEQ ID NO: 1059 - protein, 11 aa, synthetic construct
RLYFIAYPDDD

SEQ ID NO: 1060 - protein, 11 aa, synthetic construct
  MOD_RES 3: note = phosphotyrosine
RLYFIAYPDDD

SEQ ID NO: 1061 - protein, 11 aa, synthetic construct
RLYFKAYPDDD

SEQ ID NO: 1062 - protein, 11 aa, synthetic construct
  MOD_RES 3: note = acetylated lysine
RLKFIAYPDDD

SEQ ID NO: 1063 - protein, 6 aa, Homo sapiens
LVFFAE

SEQ ID NO: 1064 - protein, 6 aa, synthetic construct
LVFFAK

SEQ ID NO: 1065 - protein, 6 aa, synthetic construct
LVFFGK

SEQ ID NO: 1066 - protein, 6 aa, synthetic construct
LVFFAG

SEQ ID NO: 1067 - protein, 6 aa, synthetic construct
LVPFAE

SEQ ID NO: 1068 - protein, 8 aa, synthetic construct
EFLNRFYK

SEQ ID NO: 1069 - protein, 10 aa, synthetic construct
ELISFCLDTK

SEQ ID NO: 1070 - protein, 12 aa, synthetic construct
ENRLCYYLGATK

SEQ ID NO: 1071 - protein, 17 aa, synthetic construct
DEMARHLQEYQDLLNVK

SEQ ID NO: 1072 - protein, 12 aa, synthetic construct
LALDIEIATYRK

SEQ ID NO: 1073 - protein, 9 aa, synthetic construct
FAAWAAYAA

SEQ ID NO: 1074 - protein, 13 aa, synthetic construct
XAKLDEESILKQK

SEQ ID NO: 1075 - protein, 13 aa, synthetic construct
AAKLDEESILKQK

SEQ ID NO: 1076 - protein, 13 aa, synthetic construct
RXKLDEESILKQK

SEQ ID NO: 1077 - protein, 8 aa, synthetic construct
YRELRLLK

SEQ ID NO: 1078 - protein, 8 aa, synthetic construct
  MOD_RES 2: note = asymmetric dimethylarginine
YRELRLLK

SEQ ID NO: 1079 - protein, 8 aa, synthetic construct
  MOD_RES 2: note = symmetric dimethyl arginine
YRELRLLK

SEQ ID NO: 1080 - protein, 12 aa, synthetic construct
  MOD_RES 2: note = may be deiminated to form citrulline
LRLAFAYPDDDK

SEQ ID NO: 1081 - protein, 10 aa, synthetic construct
  MOD_RES 3: note = phosphotyrosine
RLYFIAYPDD

SEQ ID NO: 1082 - protein, 4 aa, synthetic construct
KLVF

SEQ ID NO: 1083 - protein, 4 aa, synthetic construct
LVFF

SEQ ID NO: 1084 - protein, 11 aa, synthetic construct
EIAFLKQRVWK

SEQ ID NO: 1085 - protein, 11 aa, synthetic construct
VELQEEIAFLK

SEQ ID NO: 1086 - protein, 10 aa, synthetic construct
EIAFLKQRVW

SEQ ID NO: 1087 - protein, 7 aa, synthetic construct
RLIFAYP

SEQ ID NO: 1088-1089 - skipped (2 entries)

SEQ ID NO: 1090 - protein, 5 aa, synthetic construct
RLIFA

SEQ ID NO: 1091 - protein, 17 aa, synthetic construct
DQQIASSRLAASFAAQY

SEQ ID NO: 1092 - protein, 7 aa, synthetic construct
RLAFSAL

SEQ ID NO: 1093 - protein, 6 aa, synthetic construct
EFIAWL

SEQ ID NO: 1094 - protein, 7 aa, synthetic construct
DQQRLIF

SEQ ID NO: 1095 - protein, 13 aa, synthetic construct
IVNFSRLIFHHLK

SEQ ID NO: 1096 - protein, 10 aa, synthetic construct
DIRLIFSNAK

SEQ ID NO: 1097 - protein, 16 aa, synthetic construct
GQSRLIFTYGLTNSGK

SEQ ID NO: 1098 - protein, 11 aa, synthetic construct
DEHCLRLIFLK

SEQ ID NO: 1099 - protein, 13 aa, synthetic construct
FAKLKDEESILKQ

SEQ ID NO: 1100 - protein, 13 aa, synthetic construct
YAKLKDEESILKQ

SEQ ID NO: 1101 - protein, 13 aa, synthetic construct
WAKLKDEESILKQ

SEQ ID NO: 1102 - protein, 4 aa, synthetic construct
LVFA

SEQ ID NO: 1103 - protein, 4 aa, synthetic construct
LIFA

SEQ ID NO: 1104 - protein, 4 aa, synthetic construct
LVAR

SEQ ID NO: 1105 - protein, 4 aa, synthetic construct
LAFA

SEQ ID NO: 1106 - protein, 4 aa, synthetic construct
LQAR

SEQ ID NO: 1107 - protein, 4 aa, synthetic construct
LDAA

SEQ ID NO: 1108 - protein, 4 aa, synthetic construct
LCAR

SEQ ID NO: 1109 - protein, 4 aa, synthetic construct
LGAA

SEQ ID NO: 1110 - protein, 4 aa, synthetic construct
LMFA

SEQ ID NO: 1111 - protein, 4 aa, synthetic construct
LSAR

SEQ ID NO: 1112 - protein, 4 aa, synthetic construct
LEFA

SEQ ID NO: 1113 - protein, 4 aa, synthetic construct
MFAY

SEQ ID NO: 1114 - protein, 4 aa, synthetic construct
  MOD_RES 1: note = methionine sulfoxide
MFAY

SEQ ID NO: 1115 - protein, 5 aa, synthetic construct
RLIFY

SEQ ID NO: 1116 - protein, 211 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNDAITTPDPTTNASLLTKLQAQNQWLQDMTTHLILRSFKEFLQSSLRALRQM

SEQ ID NO: 1117 - protein, 8 aa, synthetic construct
DGISALRK

SEQ ID NO: 1118 - protein, 8 aa, synthetic construct
SNMCESSK

SEQ ID NO: 1119 - protein, 12 aa, synthetic construct
EALAENNLNLPK

SEQ ID NO: 1120 - protein, 16 aa, synthetic construct
DGCFQSGFNEETCLVK

SEQ ID NO: 1121 - protein, 34 aa, synthetic construct
IITGLLEFEVYLEYLQNRFESSEEQARAVQMSTK

SEQ ID NO: 1122 - protein, 8 aa, synthetic construct
VLIQFLQK

SEQ ID NO: 1123 - protein, 11 aa, synthetic construct
DPTTNASLLTK

SEQ ID NO: 1124 - protein, 12 aa, synthetic construct
DMTTHLILRSFK

SEQ ID NO: 1125 - protein, 12 aa, synthetic construct
ACLILRSIEELK

SEQ ID NO: 1126 - protein, 12 aa, synthetic construct
SDSRNTLILRCK

SEQ ID NO: 1127 - protein, 31 aa, synthetic construct
DSSHQISALVLRAQASEILLEELQQGLSQAK

SEQ ID NO: 1128 - protein, 17 aa, synthetic construct
ARTVGIEELILRIQESK

SEQ ID NO: 1129 - protein, 12 aa, synthetic construct
STLVLRCHRRRK

SEQ ID NO: 1130 - protein, 12 aa, synthetic construct
DSPQEPLVLRLK

SEQ ID NO: 1131 - protein, 8 aa, synthetic construct
DLVLRATK

SEQ ID NO: 1132 - protein, 319 aa, synthetic construct
MRNIIKLALAGLLSVSTFAVAAEESSPEALRIGYQKGSIGMVLAKSHQLLEKYPESKISWVEFPAGPQMLEALNVGSIDLGSTGDIPPIFAQAAGADLVYVGVEPPKPKAEVILVAENSPIKTVADLKGHKVAFQKGSSSHNLLLRALRQAGLKFTDIQPTYLTPADARAAFQQGNVDAWAIWSPYYSAALLQGGVRVLKDGTDLNQTGSFYLAARPYAEKNGAFIQGVLATFSEADALTRSQREQSIALLAKTMGLPAPVIASYLDHRPPTTIKPVNAEVAALQQQTADLFYENRLVPKKVDIRQRIWQPTQLEGKQL

SEQ ID NO: 1133 - protein, 29 aa, synthetic construct
LALAGLLSVSTFAVAAESSPEALRIGYQK

SEQ ID NO: 1134 - protein, 18 aa, synthetic construct
GSSSHNLLLRALRQAGLK

SEQ ID NO: 1135 - protein, 17 aa, synthetic construct
DPYYSAALLQGGVRVLK

SEQ ID NO: 1136 - protein, 18 aa, synthetic construct
DLNQTGSFYLAARPYAEK

SEQ ID NO: 1137 - protein, 11 aa, synthetic construct
DLFYENRLVPK

SEQ ID NO: 1138 - protein, 13 aa, synthetic construct
DIRQRIWQPLEGK

SEQ ID NO: 1139 - protein, 13 aa, synthetic construct
RLQFQALMAADDD

SEQ ID NO: 1140 - protein, 13 aa, synthetic construct
LAQRQAFDAADDD

SEQ ID NO: 1141 - protein, 13 aa, synthetic construct
FAQLQARFAADDD

SEQ ID NO: 1142 - protein, 4 aa, synthetic construct
  MOD_RES 1: note = asymmetric dimethylarginine
RLAF

SEQ ID NO: 1143 - protein, 4 aa, synthetic construct
  MOD_RES 1: note = symmetric dimethyl arginine
RLAF

SEQ ID NO: 1144 - protein, 4 aa, synthetic construct
RLAF

SEQ ID NO: 1145 - protein, 4 aa, synthetic construct
  MOD_RES 2: note = may be deiminated to form citrulline
RLAF

SEQ ID NO: 1146 - protein, 158 aa, synthetic construct
QEAGGRPGADCEVCKEFLNRFYKSLIDRGVNFSLDTIEKELISFCLDTKGKENRLCYYLGATKDAATKILSEVTRPMSVHMPAMKICEKLKKLDSQICELKYEKTLDLASVDLRKMRVAELKQILHSWGEECRACAEKTDYVNLIQELAPKYAATHPK

SEQ ID NO: 1147 - protein, 138 aa, synthetic construct
LTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHK